SEQUENCE LISTING (19 sequences)

SEQ ID NO: 1 - DNA, 6648 bp, Homo sapiens
atggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcagggagtgccgtgtctggctctcactgggctgctctgatcttggctgtgctgcgcctcactcaggctgggcaggactgaccccggcggggccctgttctccgtgttggtcctgcaggggagcgaaaacgacggcccccagcttcacgctggccaccatcaagggggacgaatacaccttcacctccagtaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgactgtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaggtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtga

SEQ ID NO: 2 - protein, 2215 aa, Homo sapiens
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRECRVWLSLGCSDLGCAAPHSGWAGLTPAGPCSPCWSCRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVLSVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 3 - protein, 2175 aa, Homo sapiens
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVLSVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 4 - protein, 1200 aa, Homo sapiens
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRSVPESLLVAEWCLCQPSKRLSQAWPGFGFAAS

SEQ ID NO: 5 - protein, 1203 aa, Homo sapiens
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEVLQAQLPEGQKKSSVRHKLVHLTLKKKSKLTEEVTKRLHDGESTVQGNSMLEDRPTSNLEKLHFIIGNGILRPALRSVPESLLVAEWCLCQPSKRLSQAWPGFGFAAS

SEQ ID NO: 6 - protein, 2092 aa, Homo sapiens
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRECRVWLSLGCSDLGCAAPHSGWAGLTPAGPCSPCWSCRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVLSVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 7 - protein, 2185 aa, Homo sapiens
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRECRVWLSLGCSDLGCAAPHSGWAGLTPAGPCSPCWSCRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVLSVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 8 - protein, 2177 aa, Homo sapiens
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWASRFYEAYKFSGPSLPKNDVIVAVNWTGVYFVDEQEQVLLELSFPEIMAVSSSRECRVWLSLGCSDLGCAAPHSGWAGLTPAGPCSPCWSCRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVLSVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 9 - protein, 2166 aa, Homo sapiens
MDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVLSVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 10 - DNA, 1758 bp, Homo sapiens
atgaccatcacttacacaagccaagtggctaatgcccgcttaggctccttctcccgcctgctgctgtgctggcggggcagcatctacaagctgctatatggcgagttcttaatcttcctgctctgctactacatcatccgctttatttataggctggccctcacggaagaacaacagctgatgtttgagaaactgactctgtattgcgacagctacatccagctcatccccatttccttcgtgctgggcttctacgtgacgctggtcgtgacccgctggtggaaccagtacgagaacctgccgtggcccgaccgcctcatgagcctggtgtcgggcttcgtcgaaggcaaggacgagcaaggccggctgctgcggcgcacgctcatccgctacgccaacctgggcaacgtgctcatcctgcgcagcgtcagcaccgcagtctacaagcgcttccccagcgcccagcacctggtgcaagcaggctttatgactccggcagaacacaagcagttggagaaactgagcctaccacacaacatgttctgggtgccctgggtgtggtttgccaacctgtcaatgaaggcgtggcttggaggtcgaatccgggaccctatcctgctccagagcctgctgaacgagatgaacaccttgcgtactcagtgtggacacctgtatgcctacgactggattagtatcccactggtgtatacacaggtggtgactgtggcggtgtacagcttcttcctgacttgtctagttgggcggcagtttctgaacccagccaaggcctaccctggccatgagctggacctcgttgtgcccgtcttcacgttcctgcagttcttcttctatgttggctggctgaaggtggcagagcagctcatcaacccctttggagaggatgatgatgattttgagaccaactggattgtcgacaggaatttgcaggtgtccctgttggctgtggatgagatgcaccaggacctgcctcggatggagccggacatgtactggaataagcccgagccacagcccccctacacagctgcttccgcccagttccgtcgagcctcctttatgggctccaccttcaacatcagcctgaacaaagaggagatggagttccagcccaatcaggaggacgaggaggatgctcacgctggcatcattggccgcttcctaggcctgcagtcccatgatcaccatcctcccagggcaaactcaaggaccaaactactgtggcccaagagggaatcccttctccacgagggcctgcccaaaaaccacaaggcagccaaacagaacgttaggggccaggaagacaacaaggcctggaagcttaaggctgtggacgccttcaagtctgccccactgtatcagaggccaggctactacagtgccccacagacgcccctcagccccactcccatgttcttccccctagaaccatcagcgccgtcaaagcttcacagtgtcacaggcatagacaccaaagacaaaagcttaaagactgtgagttctggggccaagaaaagttttgaattgctctcagagagcgatggggccttgatggagcacccagaagtatctcaagtgaggaggaaaactgtggagtttaacctgacggatatgccagagatccccgaaaatcacctcaaagaacctttggaacaatcaccaaccaacatacacactacactcaaagatcacatggatccttattgggccttggaaaacagggatgaagcacattcctaa

SEQ ID NO: 11 - protein, 585 aa, Homo sapiens
MTITYTSQVANARLGSFSRLLLCWRGSIYKLLYGEFLIFLLCYYIIRFIYRLALTEEQQLMFEKLTLYCDSYIQLIPISFVLGFYVTLVVTRWWNQYENLPWPDRLMSLVSGFVEGKDEQGRLLRRTLIRYANLGNVLILRSVSTAVYKRFPSAQHLVQAGFMTPAEHKQLEKLSLPHNMFWVPWVWFANLSMKAWLGGRIRDPILLQSLLNEMNTLRTQCGHLYAYDWISIPLVYTQVVTVAVYSFFLTCLVGRQFLNPAKAYPGHELDLVVPVFTFLQFFFYVGWLKVAEQLINPFGEDDDDFETNWIVDRNLQVSLLAVDEMHQDLPRMEPDMYWNKPEPQPPYTAASAQFRRASFMGSTFNISLNKEEMEFQPNQEDEEDAHAGIIGRFLGLQSHDHHPPRANSRTKLLWPKRESLLHEGLPKNHKAAKQNVRGQEDNKAWKLKAVDAFKSAPLYQRPGYYSAPQTPLSPTPMFFPLEPSAPSKLHSVTGIDTKDKSLKTVSSGAKKSFELLSESDGALMEHPEVSQVRRKTVEFNLTDMPEIPENHLKEPLEQSPTNIHTTLKDHMDPYWALENRDEAHS

SEQ ID NO: 12 - protein, 604 aa, Homo sapiens
MFEKLTLYCDSYIQLIPISFVLGFYVTLVVTRWWNQYENLPWPDRLMSLVSGFVEGKDEQGRLLRRTLIRYANLGNVLILRSVSTAVYKRFPSAQHLVQAGFMTPAEHKQLEKLSLPHNMFWVPWVWFANLSMKAWLGGRIRDPILLQSLLNEMNTLRTQCGHLYAYDWISIPLVYTQVVTVAVYSFFLTCLVGRQFLNPAKAYPGHELDLVVPVFTFLQFFFYVGWLKVAEQLINPFGEDDDDFETNWIVDRNLQVSLLAVDEMHQDLPRMEPDMYWNKPEPQPPYTAASAQFRRASFMGSTFNISLNKEEMEFQPNQEDEEDAHAGIIGRFLGLQSHDHHPPRANSRTKLLWPKRESLLHEGLPKNHKAAKQNVRGQEDNKAWKLKAVDAFKSAPLYQRPGYYSAPQTPLSPTPMFFPLEPSAPSKLHSVTGIDTKDKSLKTVSSGAKKSFELLSESDGALMEHPEVSQVRRKTVEFNLTDMPEIPENHLKEPLEQSPTNIHTTLKDHMDPYWALENRSVLHLNQGHCIALCPTPASLALSLPFLHNFLGFHHCQSTLDLRPALAWGIYLATFTGILGKCSGPFLTSPWYHPEDFLGPGEGR

SEQ ID NO: 13 - protein, 498 aa, Homo sapiens
MFEKLTLYCDSYIQLIPISFVLGFYVTLVVTRWWNQYENLPWPDRLMSLVSGFVEGKDEQGRLLRRTLIRYANLGNVLILRSVSTAVYKRFPSAQHLVQAGFMTPAEHKQLEKLSLPHNMFWVPWVWFANLSMKAWLGGRIRDPILLQSLLNEMNTLRTQCGHLYAYDWISIPLVYTQVVTVAVYSFFLTCLVGRQFLNPAKAYPGHELDLVVPVFTFLQFFFYVGWLKVSLLAVDEMHQDLPRMEPDMYWNKPEPQPPYTAASAQFRRASFMGSTFNISLNKEEMEFQPNQEDEEDAHAGIIGRFLGLQSHDHHPPRANSRTKLLWPKRESLLHEGLPKNHKAAKQNVRGQEDNKAWKLKAVDAFKSAPLYQRPGYYSAPQTPLSPTPMFFPLEPSAPSKLHSVTGIDTKDKSLKTVSSGAKKSFELLSESDGALMEHPEVSQVRRKTVEFNLTDMPEIPENHLKEPLEQSPTNIHTTLKDHMDPYWALENRDEAHS

SEQ ID NO: 14 - DNA, 3696 bp, Homo sapiens
atgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagccaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcaaaacatgttccaaatcaagtatagatattgagaatgggtttatttctgaatctcagtatacatatgccttaaaagaaaaagcgaaatatcaatgcaaactaggatatgtaacagcagatggtgaaacatcaggatcaattagatgtgggaaagatggatggtcagctcaacccacgtgcattaaatcttgtgatatcccagtatttatgaatgccagaactaaaaatgacttcacatggtttaagctgaatgacacattggactatgaatgccatgatggttatgaaagcaatactggaagcaccactggttccatagtgtgtggttacaatggttggtctgatttacccatatgttatgaaagagaatgcgaacttcctaaaatagatgtacacttagttcctgatcgcaagaaagaccagtataaagttggagaggtgttgaaattctcctgcaaaccaggatttacaatagttggacctaattccgttcagtgctaccactttggattgtctcctgacctcccaatatgtaaagagcaagtacaatcatgtggtccacctcctgaactcctcaatgggaatgttaaggaaaaaacgaaagaagaatatggacacagtgaagtggtggaatattattgcaatcctagatttctaatgaagggacctaataaaattcaatgtgttgatggagagtggacaactttaccagtgtgtattgtggaggagagtacctgtggagatatacctgaacttgaacatggctgggcccagctttcttcccctccttattactatggagattcagtggaattcaattgctcagaatcatttacaatgattggacacagatcaattacgtgtattcatggagtatggacccaacttccccagtgtgtggcaatagataaacttaagaagtgcaaatcatcaaatttaattatacttgaggaacatttaaaaaacaagaaggaattcgatcataattctaacataaggtacagatgtagaggaaaagaaggatggatacacacagtctgcataaatggaagatgggatccagaagtgaactgctcaatggcacaaatacaattatgcccacctccacctcagattcccaattctcacaatatgacaaccacactgaattatcgggatggagaaaaagtatctgttctttgccaagaaaattatctaattcaggaaggagaagaaattacatgcaaagatggaagatggcagtcaataccactctgtgttgaaaaaattccatgttcacaaccacctcagatagaacacggaaccattaattcatccaggtcttcacaagaaagttatgcacatgggactaaattgagttatacttgtgagggtggtttcaggatatctgaagaaaatgaaacaacatgctacatgggaaaatggagttctccacctcagtgtgaaggccttccttgtaaatctccacctgagatttctcatggtgttgtagctcacatgtcagacagttatcagtatggagaagaagttacgtacaaatgttttgaaggttttggaattgatgggcctgcaattgcaaaatgcttaggagaaaaatggtctcaccctccatcatgcataaaaacagattgtctcagtttacctagctttgaaaatgccatacccatgggagagaagaaggatgtgtataaggcgggtgagcaagtgacttacacttgtgcaacatattacaaaatggatggagccagtaatgtaacatgcattaatagcagatggacaggaaggccaacatgcagagacacctcctgtgtgaatccgcccacagtacaaaatgcttatatagtgtcgagacagatgagtaaatatccatctggtgagagagtacgttatcaatgtaggagcccttatgaaatgtttggggatgaagaagtgatgtgtttaaatggaaactggacggaaccacctcaatgcaaagattctacaggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacacattgcgaacaacatgttgggatgggaaactggagtatccaacttgtgcaaaaagatag

SEQ ID NO: 15 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 16 - DNA, 135313 bp, Homo sapiens
agtccccagtctttgcttaggcccctacgtacacaaactgaacctagtgacccagcatggcctctaatttctcaacacttctgtacttctgtaatgattaacccatgcttctcacagatccatgccccaaatttctgtgaataggccctgactggcccagctaagatcatgtgactgcacatgaccagtccactttggcattaacaagcctactgcagactcttcccttggtgttggagtcactcctagaaaagagcaaatctttgtgagccaggcagtcaacctgctggcagcttccactcagccttggagttttttctatgtgtaactttcataaactgagccttatttatttattttttgcactatcatctcatgaaatattattgcgtaagctgaggaaacatgttattcatgatgactggagtttcaagttttaattgtacaatgatttagttttgagtttggtagaaataaaatcaaatttaaaaatcagatatttttcatcttacattatgatgtcccaaaactgcctttatgcttgtgacatagattcataatgtcttctcattccacctgtaatcactgtttgaaataaacatatgtctaatgatatatttggggacattctattttcttcagcttgttgcaagtgaattgatggtgatcttttggtattggtttcattatcaaatttatctccactccaaaattacagtaatttcaaagtaatttagtctatatatttttccatagcttttcttccaaatagaaactgtaaaaagttataaattacttctctccactactgaatttttgtttgcagaataactgatgtaagtagcagaatgcctcttcctagttcaaccctcaggaatagaagtgagaagatctttaaaacttcaccattttccttgacttgtttataattctgaatgtaaatgtgaattgatatggtctatcgcttaacaccacaactcttaatctatgtgcagggtcgtagctcaaaactactgccaggaccacatcaatttcatattcaccctgatcaatgtgtattaatggtgataactatgagaatgaaatgtacagttatcagtatcatttttgactcactaggtatatcctcagaaatatatgaaaaaactaaacacagcttttagtttgacataatttttaaacaactggagttaccttgggagaaaaatcctaccaaatatctataatattgaaagagtaaaaaagagttaaatgtccttaacatcattaatcattagggacatgcaaatcaaaaccacagtgaaataccatctcacaccctttaggatggtggtgataagaagaaaaacagagcataacaagtgttggccaggatgtggaaaagctggaaccattgtgcactgctgattggaaagtacaatggtgcagctgctaagggaaatagtatggtagttcttcaaaaaataaacagttataccatttgattcagcagtttcactcctaggtatataccccaaagaattgaaagcagaatctcaaatatttgtacacctatgttcatagcagcattactcacaatagccaaaaggtggaaacaacccgaatgaccctggatggacgaatggataaacaaaatgaggtctatactgacaataggatattaattgaccttaaaaaggaaagaaattctggccgggcacggtggctcacacctgtaataccagcactttgggaggccaaggcaggcagatcacctgaggttgggagtttgagaccagcctgaccaacatggagaaacctcatatctacttaaaatacaaaaaaaaaaattagccaagcatggtggcgcctgcctgtaatcccaggtactcagtaggctgaggcaggagaatcgcttgaacaggaagcagaggttgcaatgagctgagattgcaccatcacactccagcctgggcaacaagagtgaaactgcatctcaaaaaaaaaaaaaacaaaaaaaacaaaaaaagaaattctgacacatctgctatggtctaaattatgtgttcctctaaaattcataaattgaaatcctaacccccaaggtgatggtattaggaggtgaggctttgtggaggtgattaggtcatgagggtacaaccctcgtgaatgggactagtgccctcataaaaagaagcccaagagagaccccttttcccttccactggatgaggtcacaccaagaagttaccatctacatgtcaggaaataggccctcaccagacaccaaatctattggcaccttgatcttggacttcccagcctgcaaagctgtgagaaataagttcctgttgtttataaaccacccagtttatggtattttgttatagcagctcaaacagattaagatggcttgctacaacatagatgaactttaaagattatgcttttctatgattccacttagatggggtaccaagagtagtcaagttcattgagacagaaaatagagtggttggcaggggctagggggagggaactctggggagttagtgtttaatggatacagagtttcagttttgcaaaatgaaaaagttctgaagatggatggtgctgatggctgcacaatatgaatgtatcaacactactcaactgtacacttaaaataaggttcaaatgatacattttatttcatgtgtgtgtcaatctcaacaaacagatttgttcaggcaaggaaactggttagatgcgaataatactattagagcatcatcaattgaatattaacaaagtgctcatagtttaactttctagctcaaggaagaatggaccattttgaaactatgacagaacattacttatatagctgatgtctttgggaattggaaggaggcatattccttcaccagctgtggctccccttcagcaacctcatatactctccaagcttctctttcctgggtcacctgtttaatcactcccgggacttaatcttccacctatatgttgaccactcacaaatctatgtctccatctcacaagcttattcttgactccagacccaagtattcaactgcctgctgaatacgtgtggtcagatgtcatagaacttcagcttcagtatatcaaatgcaaacccctgttccccccaactgcctcctactccccactggccttcctctggcattccctcctcagttatgagcaccaccgtctcactagccagccagtcaagccccaaactccatctagctgacttctgcctcttcctcaccaccctcttccagtaactcatcaggcactgctgtgtctcattccttcctatccctccagtccctccccttctctccatcatggctgtcactgcatggttcaggctctctggctccccccaaaccacccccacattgctgccgaggtgaactgactactcttggcagccactggattaaaatctttcatcatcttcagcatgataaaacccatatcctttagcatgtaacaaggtcttaatgattctgccagagcttgcttgggggtagcctgcacttgtgggccactccagtcacttcacaggtgctcagtaaatctcagttgaatcagtcatcatcatcatcatcatcatcatcatcatcatcatcatcaatttttcagtctggttcctgtctccttttccagcatcctccattcatagcctcatagccttcactccagccatgtttcacttgtggttttcctgggcaagataagctattcctccctgtctttgcagagtttaaatgactcacttgttcaagtacccaccgttgccatgtgggaccgtgagcaaagtacttaatctcactaagcttcacgttcctcatctgtaaaacagcaaatatggacctcacaaaattgtagtgaggctaaaatgaaataacatatgcaaaagcagtttataaataataaacttactataaaatattattttgtaattctgcaagcttgtcttaaatgccatcacctccaaggagcctttttgccatcataagcagaaactatctctctcttcttggaagctccaccatgcacagcctatgggccctcatcacactccttgagttattcgagttcaagtcccgtgtttacaaccagaccgcaaactctatgaagtcagcatccattcctctctgtggttctccctccgccccatccaggtctcaagggtctagagtctttcaaagagaacacattctgagatttgaggaggcagagacaaaaagttccactgcgaagtgccagggaggcttctgtttggggtgtcccttgggatcacagatcccccacctggtgatgagtcaacccagcaccaccccattgcagggctggaatgacagtaatgggcccacctgctgcctctcctcatacccgcaccccagtcagacattgcaagtcagtcacggctctgtcctgctgggcctggagtgttccagtgccttttccatcacagcaccaagcagccactactagtcgatcaatttcagcacaagagataaacatcattaccctctgctaagctcagagataacccaactagctgaccataatgacttcagtcattacggagcaagataaaagactaaaagagggagggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattagcatgggcttcgtgagacagatacagcttttgctctggaagaactggaccctgcggaaaaggcaaaaggtaacagttactgtctgtggtttaaaaatgaggtgtggagcaaataaacaggttggaagtgtggggtggggtggtggggtagggtggtggggcagggtggggggttgtgagcagtcagtgggcttgtcgccgattagcactgaagcagtgtttagctggacggcctttctgtgggcccctctgacagtgcccttcccaggaagatgtgtttctctgtcctcagccacatgaaaatcttttgcctaccgtgcctgtcaatccattgcctgcccgcccctcccccaccccccgttttacacctgcctgtccagtctaccgctctctagggcatccacgctgagcagtgggaagaactttaagccctgaagagcaggccaaaggcaagcaagaaccccctcgaacagcttcccagcttagtgaggccttatttcattgattctctgaggcacattgttttttcacatgttagcatttctgaaattgggatgcagctcacgatcaagtcacagtttaactggacacattatttttctttcttagtggtgcagaaaagtaacagtgtgtcttacaattgactgcgtcctagattctgtgagatgcaatacgttattaaccatcacgcacatttcctgaactctttcaatgagcagacaccagcctgggttagactggagccctaaaagcacgacacagattccaccctggactggcttctgttctgcctgggaaaacccaaagtacgtttggagaccaagagcaacataaagtagcataggtggaatagtccatgagaagtgcgagcaaaaggtgccggagatcagagaacaccaagactgtacttgtaaatgacaactggctttgtgcaattttttctgggaaaggataaggagtgactatagaactgtaaagaaagaatgcactttgctacagccttgcagagttgtgcaaatgccgatgactaaaggagctgaaagaggaaggaggggataagggatgggggctgggtaggggtgagattaggaccctgggagctgcaagccactggagagatcaggaggaaagggagggagacctgctttaggcgagaagagaacagtatttgttccaaatctcggttcagaataagttcatgtaggtgatggggccaactggaacaggtgaaggcctatgaatgagtgtctcagttagggtctccttagagtttaatatgaaaaggtgttagctaagtacagagctggtacctgagagagtaaaaggaaactctaaggtatcatggaggtagcaattgcaggacacagctcccacccctagggctgagagaaccaagggaagagacaggaattattaagacttggagcatagatgagaggtctgtggagctgacattaggacttgggaggaaggcgtgcatggaggctgctgctggatctctgaacctgacctcgggtctggacccctgaggagaaagccctggcaggttggtgcatgtggggccgagggacaatagcttaacaaccagcataaaagagagcagcatgggacacgcttcaaccatgcgcatggatggctccaaaacctgtgtgtggctggcccaggacgcagggaggctgcagggggaagagacaagttaaacctgacttgtctgggaagcaccattgtcctcaggtcactttcctctgtcaagcctggtgctgaagttatctgttgtctccaggggccaagtattaagagtaatcagaaactcagtcctttcttctaggagcttcccttcttgcatgaaaatcctgataaaactggaaaaaaaaacctcatgattaaattttttcatgtattcattctttccttctatcaaaaaataatctccaggcaccgtgctaggttcattggtatacaatggcaacaagacctcccagcccctgcctatgtgaggcatctgtggactgcggaggaaaatccaatatgccattgttctctctttcccataagaaattacaattctcagttcattttattctcactgtgctctttgtgaccctcaaagggggtcacatgataacaggactgtagctgctggcctaaaatgagcccattcctgtggcgctcatgtcgctgtgacagagaataaccctgttttcagaatgctctggtgccctccctctcaatctggcctttcgctggcatgggtgggcgactcctgctcagggactctgccttctccacagtgtgctcccagggagatggagccactcgggctgagggccttggccagggcacctcccagggctgggcctggtctgggctggcgttcactggatgccatcctgatggcctggaaattgagatttctgtctggcacgcctcccgatggctccccacctgctaccacattccaggagcttccaggatgtctgggtaagacagaggcacccccaacagattcagtagctctgagagggatctgtggctccttcctaagcttgcggttcttctggaaacttctgcctctagaagatggtccctctaagaaaagtacaaccacccagcccataattcagctcccaggttttccctcaaacctccatgtctcctgtaagcagagcaagagtaaaatcagataccaaatttcctcattcctcagctcccaatccctaagggcataagatgaaaatcttcagatctctgctttcctccctctttttttcttcctctgttaacatttgtcaagtgttactaagtgtctggcactgtactaagtgcatcacctccctgaactctccgaacagttccacgagagaggcctctctgtgatccccccggtactgatgaggtcactgaggctccagagaaggattagtaactggtggggttggacctgggattcacacccatgctgcgtgacccaggacaggcaggcatggccgttacaccacactgacccccgtggatcgagatctatccaatagtctggtcactgatatcactaagatagagtggccatataatttatcatccaatcagggcagttttgcaagtgaaagggagcactattaataattgcactgggacaataaatgtaaaccaacactggacctggaaaactgggacgtgtgtttgccctataccaaggtaagctagacacagccactgccttcatggagttcagaaccaggcaggggcggctcccacgtataattactgtgcagcacaacgtggagaccgtggagtagaaggaaacacggatgggaggtgaggaggaggtctgtgagctcagaggaggcaccggggctggagagggtgagagaagacttcccaaggagttcatcctgataacgtgcattcccaatgacgagcgctctctccactgcacaagacaagtatacatctgcccgtgttggctgtggacctggcgctgtgtcagggagggtttatgaagatcactaggtgggtctcttggtgtcatcccttcatcccagcttctgggttaggatggatatctgtgggggggcctgaggactcatgaaagtggggcgctaatcatgttttggacaccacaccctggagcacctgggacagctgtggcctttgtcctgggttcagcatcaagccgaggatgtggcaagtaaagagaggctgggcaccaactccagtgtacccaggctccgggtcatgtttgtccaggctaagaattctgtcctggttctcagtgcagaaggaagaatcatggggctcattttaggccttggctgccttctgttaaattgaaaacagagcaggaaggaagaaaatttaacaggctcagttctaaaacaacaagcacaactgtgcccttgccagaaacccctcctccccatgttgattgaatggtaaagagaggaggggaggtgagagggagagagagagagaggaagagagagagaaaggaaagaaaggaaagaagaagaaagaaagaaaaggaaagaaagaaagaaagaaagaaagaaagaaagaaagaaagaaagaaagaaagaaagagaaagaaagaaaggagggagggagggaaggggaaaagaaaagaaaagaaaaagaaaaaaagaaggaaataccagtttgggaaaaaagaattttccaccagcccttctgagccttggctgggcttaattaaagttacagacatgtgtaaagggcagggtagggggagtctgagctgctgagaaaacatgtttttaattatactgtggaatttctccctggggtatgcctgtacgcagttaagcgtcaaggacagggatgccgctctggggaggggaagctgagcatgattttggaagccggcagaagaggctattgtgaaaaccagacctgtcaggctaggaaaagaatggctggtggtctttgaccagggagtgacgcgtgaaatgcagcaaccgcccccgccccccgccaaaaacaaacacactctcacagagttagaacaacagtgacctctcaacaaatatttttcaaagattaccaaccaaccattacctagagcagcggttctcaaccttggctgcacggtggaactacctgagacgtgttaaaaagaagaaccctgatgtcccatgccccaagattctgatgtagttgatctggggtatgatctgagaccccggcatgttttcagcctgcagccacatgagaagtgctgacctaatcaacaggggtgatgatttgaggggcggggactataggcaaaaaaaaacagcctaattcaaggatgagaagagggcacaggtgaggtgggaacagtcctagggccagacaaagaaggaagggagaaaggaggtgctgatccctcccctactcctgagaggaggcctttaagtcaccgtgccttgtggagaccagattcttcaaaaatacaagaatgagtgagtgagggagtgggtggatgccaggagagtgcgtgacaagccttgcaagggaggatgacaatgcactagcttggtttggaaattttacccctggaacaggcaggccaagctggctggtcccctccctgatacacagccctccctctttatatatggagcaggggacggtgtgtggctggtttcttagcaagcaccatggttccaagttggcaactggggagttctgaatccaaaaaggagggagatgaacgtaagtggagggcaggcctacaaggttgcagataagcttaattctgtctccttactcttctgcctttgcaacaaccctgtgatcttgcgacaaccctgtaaggcaataacaaatggctcatgtttattgagtgttacctcatgccatattgtgctttcgtgtttaacacaattgtctcatttcaccctcacgactgctctgggaggtaggtcctggtatcacatccatttcacagatgagaccatttggcacggaagagttgagtgggctgcccaaggtcacatagctaagatggaacaggctggataggaaccccagtaacttgacctcagagtaaccttctcttaaccctgagtgtacactgtaggaaaaatgagcagtcccatttcagagaggacaaaactgagactcagaggttaagcaagccccaaagtggttgttaacccagatcttcccactaactcccaaatcagcatcagtgtttaacgtaccagacctctcccagatagatgttgccgcatggaagacagccgatctacgtgatagaaagccaatattgcaagcagtcgtctaaaggagtcaaatgtgttggatttgaactggatgtctcatttctttggtgaagacactggaaacaacttccaggtttcatcaattgctcctatcactcaacgttgctatcttactgaacttgttccccagccttacccactgatggaatgatccagaatggaagacaagacaccaatgtacatgaccctgggggaggctgtttcttaaatctacagactgttggtgacctgagccccatgtcaccaaaggctttcctggagaagcctcctagaccagtcttgacaaaggctcactcattccgtggatatttattgggcacctattatgagttctgccccatgtggggtgctggaatcacagtagtgacaacgacagatgaggttcctgtcctcaggaagcttactgcccttgagggcttcacttacttggaggagtgatgaacctgaagtgcggtgtgtgttaagaagcggaagtccagggccaggcgcggtggctcacgcctgtaatcccagcactttgggaggctgaggcaggcggatcaccaggtcaggagatcgagaccatcctggctaacatggtgaaaccccgtctctactaaaaatacaaaaaaattagccgggcatggtggtgggcacctgcagtcccagctactcaggaggctgaggcaggagagtggcgtgaacctgggaggcagagcttgcagtgagccaagatcgtgccactgcactccagcctgggcaacagagtgagactccgtctcaaaaagaaaaaaaaaagtgcctcacggagagtctattcttttcttcccatattgtgtgtgtgtgtgcgcgcttcctccaacacatcctccctatatatattttgagtaaaacatcttgtaaaaagttacagctacataatcaccacctgtccctaaatagtttttgctttttctttcttcaatgcacgatcattttcccccatcaatttattttttagtttcttataatcttgttgccagtaggctgttttttaaaaagcagaacatggtttgttcttactagcaggaaaggagcatttattgagcctctgctatggtgtcttttattttgctgagagcctatttacatttctttgagaggaaaacaacaaagggttacatgaaagaccatgtgaatagcccctagctgatctattaaacttgctattccccggccagctgcttcagatctccttcagatcttatgtgtttccttcctaaggtccctggagtaagggttgcatagacctattctactctccaactcacatgtccctctccctcttcctctccataattccacatctccaacccccacccctatgtgcaatgccacagggtgtggactgccacagccactggatctgcttttggaatcaagagtccttaagctccaaatggaaccgaaatttaaataccaactttcaaccatatgttaacatcagcagcctcttccaatgtaaaaacccatggcagtgtgccctgctttgtttctttaagcaatagaaacttgaaggaagcatgttggtaggccagatttttgttggctttgcaatggatcacagtcatttattcactcattcattcactgattcattaaatgaccacatttgcaagggcaaggtaatggggagggccagaaaggacactggccccagaaacaggaggctggattttggttctgatgctgccactgctgatgtgacactgcacaggtcacctgcctcctctgagcctctttccttaactgcagagtgagtggctacagagaaatctttactacctgttagatcagcattacctgggagcttgttagaaatgcaagctctggtggggccatactgaacccaaatctgcattcatgtgcatagtgacagctaaaatgcactgaagcagatgatcttgatgatcctttatgaaagtctcatgctaatgcagttttctaaaatagaggcagagtggaacccagatggacacaaaatctggttgatataataaaacaaggtagagggtgtatggtggggagggggtaaaggaaggaaactgtttaggtaaagataccacaaccaaagtcctactgcacacatgggatctgaggagggctgtgtctgctctggttacgttttctataatctcttagcaccactgaactttctctctttttgttttgtttttccagattcgctttgtggtggaactcgtgtggcctttatctttatttctggtcttgatctggttaaggaatgccaacccgctctacagccatcatgaatgtaagcatagcagggtagcttgggcaagccctgaagagactttggtctgggccttttgtctagaaagatcttggggtgggagtgtggggatcagatctgcttatcatcatttcatgtctatgatgcatgtaacagatttatcaatgttacacaaattataatttttaaaaagtctttagagacagggtctcactctgttgccgaggctggagtacagtgttaggaccatggcacactgcagcttctatctcttgggctcaagtgatcctcctgcctgggcttccaaagtgctggaattataggcatgagccactgctcccagctaatttttttgttttttgtggagacagagtcactacattgcccgggctggtcttgaactcctggcctcaagtgatcctcccacctcagcgttctaaagcactgggattacaagcatgagccaccttgtccagcccaaattttcatgttttaatcctacacattctaagcaaatacttgtgtgtagttactaagggactgtgcacttatttttgtttgctttgttgttgctagtttttatttttttatacctaaactctctcgttttaaagagaacagatttgtagatgagttctcgaaaatatttcaggaatcaatatagagaatatgttatacatggtgccagagaaaaatgaggacaagagatgctatacaatcgtactgaagaaaaattttatttcttggacccctgaggtgtctgcagacctgaaaggaacctagtgagagcctcttttacactctgcccctgtgggaaagccttcacctggtttccggccctctatgtggtgaatgtggaagcctcaagcgttatgcaaatctgcccagtcctctattcttgatcttcaccttctcgttcatgagtttcaggccccagttctgaatcagcctcctgtccatcagactcttctttacctctccccgaggagcccataacctgcagccctactgcatgcttggggtaggtgctcagttcaccgtggttgaaggaatagacgagcgtctgctcaagcagcagcagcaactgcgtggagtcttcttgaactaacactcctatgcccctctcggcacaaaatgacgtgtccccccttgcttccccttcacatttccacccatgcctattacaacatccgtctgtctccccactacaccgggagcttgagagaagaggccatgtctctagcacccagcacagggactggcacacatgagatgctcctgcttcttaaatgctgagaatgaaggaggacatcagaggggcccgggccccttcccaaaaaggccaactcctaggtctgcatcctgcttggtctccatgactaatcccgtcttgtcctcattttctgttttaaaggccatttccccaacaaggcgatgccctcagcaggaatgctgccgtggctccaggggatcttctgcaatgtgaacaatccctgttttcaaagccccaccccaggagaatctcctggaattgtgtcaaactataacaactccatgtaagtgttgagatccctaccatgcaggggaggaagttgcacaccccttcacgtgctgaaatgcacacgtgcgtgcacggagcatggagcactgagtgttcttgtggctttgctgagcccctaacctcttaggagcagagcaggtttcctctctggaacattctgttaactgtcagggcacttggggagaaatctccaagctaaggccacgtgcacaaaatttcttggtccttatatccccagaatgtgacctggagtctgatggcagcccgctgcagagatgtgtccactgccttctggtcattgacctgcttgggtggagtgaatcattgtaggagaaaaactcagttccctcaccctgatcaacctggacagatctctcttcctttaaaagctttcttggacatctaagggctaggaaaaatgtcagggagcattgggaaggtaaatgaagtcaggtttacaaagtcaagtttacttcttgggagaaaaatacaatttccaaatcctctgttataattgccatcggccccctggagtggtgagatctcggaatatggctcgggtgcagtggctcttcactgtgggcctgcaggctattctgaaaagctgatgaaaaccaatgacccctcttccaagaaaaatggccacataccaaacattacactgtacatctgatttcagggaattgtagatgccaggttagtagcctcaggtctagggtcaaaattcaagtcgaatcccacaggaagagggtctgccttcggaattccctttcagagcattgggagaacatcatgggagcatattctagagacagaggcttagggtgtggacagggccatccctcacccactgtgctgaccttaagcagcaccttgtgcagcccatacctgaaggccaccagcaaaggcctgttggggagcaggctttacccgacctgtataaacaccaggctaggtgaaaactgagatacctggttactttagttttttccttgggggagctcagtatgattcttccaggagaagcctgcttttagactaaaaagaaaaaaagtttgataggtcaacctaatgattggaggtggccttccccactgtgaacaaactatggctgcatgtgccctacaatggcagagttgagtagttgtgatagagactgtatgatctgtaagcctgtaatttttatgtttgctgacccctggattaccagatgatagaagaggaaacatctgtcttcctagcaaagtcaaggaagtggcatttagcaggactcatattgctgcaagcactgccttgcagttttagtttacaactgcactttcagcttaagaaacacctgcccatccagagagatcgtgtggggtcacatggtgggatcagggaggcctgaagacagctcagtggaggctgcatggagctttggtgggaacggccctggcagtgtctatagatgttattgcggaaaactgaggggtgggagttggagaagggggctccagactctagctgtacttggcatttgaacccggaaagttgggtttcatgttttgcactcacattatgagtgaaatattggcttattcaaggttcttttgcttgcaaggcacggaaacccattcaagcaatcttaaaccccagaaggaaatctatgatttggatactagacattctcacagagccaagggcagcaaggcggggctcaggagaggcaggccaagacctggagagctgtcaggagctgcttcctcaactctcttccatctgggcctgccagccctggcctctgtatctactccattcacctctctccatggaccagtctcccctgctcctcaatgcctgggctgccattgttcatgcaattcacaatacctcggcctgggcaatcagaagctcatctctgaacaccatccaaattcctgggaacaaatcgggttgacccagctttattctccctgtcccatcagccttggcagaggcgtgcatgtgcatgcgtgccaatgtgtgtgtgcagggaggtccttgtggatgaagcatggctgtcagagcctacctgcgtgaatgggtggaagggcaggtctcagagaattgggtaaaaactggataaaccctccagtgatatccaccaatgtcaccctgtttaaggcttctctgggcaagagacacacagagcatgggaccgagaggcgagcagaccctgccaaaactgggagactgaatagatcgctcaccatccttgtcagttagcctatatgtacaaggaagtaaaattatctctttctcctgccttggcagtattgtaaggatactcaatgtagtagctaggccagacacatagtatctttaaatatagcatgagatggccaagcacggtggctcatgcctgtaatcccagcactttgggaggctgaggcgggtggatcacgaggtcaggagatcgagaccatcctggctaacacgatgaagccccgtctctactaaaaatataaaaaattagctgggtgtggtggcgggcgcctgtagtcccagctactcgggaggctgaggcaggagaatagcgtgaacccggggggcagagcttgcagtgagccgagatcacgccactgcactccagcctgggtgacagagcgagataaaaaaaaaaaatagcatgagatattattactgttataaaaataacagctatttccttattaatgaggctttgtccttacagcttggcaagggtatatcgagattttcaagaactcctcatgaatgcaccagagagccagcaccttggccgtatttggacagagctacacatcttgtcccaattcatggacaccctccggactcacccggagagaattgcaggtaagcatgactgcagtgctctcaagcatcatttccctcacctatggagagactgaagatataggaaagaacagggagagttggtgaaaaatatactagcggaggcaggaagggatggggtctggaggcggcttgaacatcaccttggtgaagatgcctcttcctccacagaagcctggaaggtaggaagttgggaaggaaggcaggaaaggtctcatccacgttaagtctagagacagaaagaatgctaagagagatggcactatgggaagtatgaggctaggtcaagggctagaagcaggggagacgagtttacagagtttcgtaaagatatagagcaactctcacagagttctagagcgagagctaaccaggaacatgaagcagcaaggccaactatcattaaggagccagggaggtcagagatcatgtattatcatgacataaatatgcataattgtactatttctcccagtaatatttagcacccaggccccgaggcagagcaagtggagagtgggtgatgcagggctgggggtgtgtatggaggcaccacagaaggtcaacaggcagcgggctgaaggcagggactggactacatgcatcaagtccaggctgcacgaggaaggatgagaaggcagatgagcacggaaatggactgggggaaatgaagaggcaagggaatagaagtctcagtgggtgccatgaccctgtttaagtgattgagaaaatgaacaagatgaaaaggttaatggctgtggtcagaaagtgaaatatgtgaattcaggatttcgaaggtagggtgggtgatgactggcccccagatgcggccatggtgaagtggggcaaaggtgcaggtgcatggtgaggggaaggaggaaatgggaggtgatgatgttggccccacacggacaccacggttgtgcaggaagatggcaggagctgggcaccagggtgggagccacctggagtcaggaagagtgaagagaaaggatgaagaggctccctctcctgtgtctctcctccccaggagaagaacaagaaacaatccgaaagtaataacaccaatgtgcctttacaaagtgtgagtgggtgttgtgtgctgtcacgtgtgtagtaggctcctctgtggatggctagagggactggacatggccactggatcccacttgcaagagcagaggaaaagagtggtcgtgaggaagtaaagccccccaaaatccaggggttgctgcagctttgggtgtggagcgtgccctctgaggaaaggctgctctgggggagattgcccaggaaacggggctcagaggccacgaaagcagctgttaggggcttctgggagatgtgtgctcctaggattagggagttgactctaaggatgaccttagaggttaacagggatgagaaaggggtcaccaaggggtctaccaggggaatgggagaggctgtattgatagaacagcttctgctgcaggttccaaacaagaaatgtgggagaatggttgaaatcagccccgggggcaccttcccgtgcatgcgtgcagctccttcaacattcagtcgaccttcagtgcctcctgtgagccaggcactgggctagtctctgggggtggagagatgagtcaggcaaatgccagccctcagagggctcacagggcagaaggtgagagatgagtgagcagaaaatgaccacagcgcgtgtggggcccagtggagggaaggaggggattcaggagcacaggagagtcaacaggggaaacttctccgaggagaatctgatcctcctcccatctggccaccttctgaagccctctctccccatccaagtgagaaaggacaggcgtatgaccagattggtgtatgaagatgctgaattacgttctcattgtttcaaactagtaaaccatagattttatgtagtaacttctacaaactgcattacaaacactccattctttgttgccctgggtagaagtttattttagtgagcccaagtttgaggaaccttatatggtatgagtacaattaccattttaatagtaagaaatcccccttcccctgtgtaccaaccagaaggtgtttttttcctaatttaaacaaacagatgcagacgtgggctgtccagctcctggcgggatgacatacctcatgcatccagtgggtttgatgatgaggcagacatttcacttaagtgcctgatcatcagattgagtcctgctgggaggaagtgtgaaggaagtaatttcaaaccacagtttctctgtggcttttacaatgtggatatgagaaccaaaatcactacttcttaaccccagagcaggactgattttgaattggtatgcaggcggttccttctgcaggcttcgggctgtgagaagtccctaacagagcaaatctggggacaagggctcaggaaaggttggccacggccccctaggaatgggggctctgcaagatccctggccttagaggctgtgagagggaacaggggtccatccccaagtaagggacacggtctttgaggaaatcccaggccagggcctgaagggcactgtcaggaacacaggctgtttcagtctgttgagattcaccggggcgctgctcactgtgagcacggactcctcaggccaatgtggcagaagagcccacctttgaaagcgagcgggtgggggtggcggggctggtgctggtgcgtgcttctgcacagccacctgggaaggtatgccgctggttgacccaggcagaggttttctttcatggcaaacctgcagtactgcattctcagcagggaggattaatggtaaaagaccaggcatggagcccccttccctctccctcgaagcaagctctgtggtctctcaatcatctttaaaacaccttcttcccgggagcctcctacattctcctggcttccctcccacccccaccctcagctcctggggcctcagcagccccacccccaagcctctaatcttcccagggaagggaacaagaagaaccacattttaaacgaaatttatttttctttcctcaggctcccagttcacatttctccctcaggagtctagggaagcttctgtctggtatcggcctcctcttcacctgggcccccgccctcctcaggtgtaccagaagccagcacactcccccttcccccccagagccacagcagccctgtctcctgggtggtcttgtgtgccaagcctgggcaacatcactcccagcttttcttgttttgccccttctccccagcaagatatttgtatgtaaggtcaggtgagtgagttaaagaataacgaagagataaacagtcaaatggagtcctgactgtcaggtcaagacaacagttatttactgaatgcctcatgtcattcaacagacatttattgagactctgattggatgtcagtctttaatgctgggtgtcagagagaggtgacttcaagggcttgcatctgtgcacccagcattgctaggtacaatgaggagtataataaaagcaggagccatagcccccaactctcaagagatctcccatgtgtgtatgtctgcatatgcgtgcgtgtgcatgtgtgcgcatgtgtgcatgtgtgtgtgcatgtgtgtgcatgcgtgtgtgtgtgcgtgtgttggggatggtgttggtggagtgagagtgtacaaggctgtgtatgaaggggtaattgggaaaagaacaatggagctggcacccagggacaggaggaaaagcaggagggctgggtttggaagacagccggatttatgtttttgaagagggaagactagaatataagggagcagcccttctcagagccctcctcctcccttcgggccctgtgtccagctttccccaaagtccttggatctttcctatgcaaaggggagtgacagtgggcaccactctcagggaacccattactgtgagagaagccactgtgccactgtgtggtcgaacttcaagaccggcttcccctgccccagctgcatggacaggcctgtggggttggcgcaagacccttccagaggaaactagctgcaacataaatccggatatggtgctgttcagggaaaggcacaacctggggatgagaagggtggctgtccagcacacaggggcaggcctcttggccactgggggaggggagaatttggagaggaagaggatgggatgccgtggaattgggaccaggaaagaatggggacatgtgatggttaaagctagttagagaagaactgggagataaacagtcacccatgcccctgaagcactcggggtgaagagattggcattttcacgcaccccagtgctttccctttgtgttgaagtcccttcgtagacatccaggcccataaggctcttctctggccagagcctcatgaactatagcactagcagggttgaggccaagcattggccctggaagccagccgaggaggagggtgcttgtgtgaatctcccaggaggggtaagaattatattaattcgatcataataagcatttattgagtgctgttttgaggcctgggagctaagcacttcacattccttaccccgcatcaacaatcctatgaggtagatgtggaaaatgcagacacggggacaggctcaatcacttgccccaaggtcaccttaactgttaggtgttctttatgcctccttataaagaaaccctgcttcccacaggtgttgagaggagctggagggagcttgactagggctcatcaggcaagccccggcatgtgcctggctctcctctttctacctggagcttttcctgcccttaatggccccaactcatttctcttagtccatgtcagtgccctgagcatctcagcccaagctgagatgatagaaacacccagaggggtcctctaccctgtgacagctgcggtgtgggaagagcacgtgtctcctccaatcctagaccagagtttctcagcctcagcatcactgacacttggggctagataatcctttgtgtgggggagggaggagtgtcttgggccttgcaggatgtttagcagcatctctggcctctacccaccagcacctccccagttgtgacacccagaaatgtctttagatcttgccaaatatttccaggaggatgaaattcccctgtttcagttccccagccccacctcaatgagaagcactgtcctagaccaaccccacaaagcatctgacacccccatccagccctggctaactttttccaccttcttactaaattgggcccagctgcttcagcagtcaatgtgttgggggcagcccactggcaagagcctcacctctaggggctcccagagaccccaagaacagaaccttcctctgagagttgagttacaagtgtttccaatcgactctggctgttttcctttttttgacccatttccccttcaacaccctgttctttctcttattcatatgtaggaagaggaatacgaataagggatatcttgaaagatgaagaaacactgacactatttctcattaaaaacatcggcctgtctgactcagtggtctaccttctgatcaactctcaagtccgtccagagcaggtagggggatgtcactggccagtggtccctggaggggagggaagcacccagcctgagaaaggcaagaaatatattggcttttttcttctttcttccttgtgttcacattcagaatccatcacttaatgccttgtatttagaaaaaaaccgggggatcacttgagatcgtgatcattttcaacataggattcgaagctgtacacatcctggtgaccttaaaacatctcaggtttttataactggaaggaaccttagagatcatggggcacaaccttctctttatagatgaggaaacagaaatctattcatttattactcaaatatttagggacagttgtaggtactagaacacagtgtgaaccagacaggcaaaaccccaggccagggagcttccattccagtggggccacaggcgatgctcaggtaagcagagactccgctgtgtgacttctggctgtgatgggtgctgcaaggaaaatccggtagagtcgagggttagagagggacggaggggcaggtttaagggggatgctcaggaaggccttcctgaggaggtggtatttgagcagagttgtctgtcagccacacagtaagtgagaggggagttccgggcttggaagctgccagcacagtgctggcaagtgctggggtggcgtcccgaggctacagaacctgagatgctgcagaagagcccacttctgctttcctggaccacttccttctcagcaccaggcaaactccttcttctatcccctggcacatttctgacctgtgtatacgcccccaatttatctaacccctttaaataatctcctctatttatgcagagcattcttaccactaactcacgacttgcacatcccttagctcccttactcctcacaacaatcctgagatgggtcagagaaggaggcttgcgcgtctggtgatggggtgatttgtgcacagttacagggctagaaattgtcagagccagatggaatccaggtcctctcaatcctaatccagtgtttcttacttcagtcctgtggctctcaaagcccagagaccagcagcatcagcgatgcctgggagcttgttaggaatgcaaattatcagggcccactccaggtgaactgggtccaaagccctgggataaggcctagcaatctgtgcttcacaagccctccaggtgattccgcaggctcaggtgtgagagctgcagctgtcctctgggccttctgggctccccgcccagcttcttcagtgtgatgaacacagcgagaatgctagatctgcagcagctgatatcccagacaccctcccgactccctcctggctgggtctgatcctcctccagactccaggagagaacgagacataaacagaacttcagagcctgtgttaaccctgagatcaaggtctgcacagggtgctgtctgagtccagaggagtgagggaccccaccccacctggtcagcaccagctcctggaagcaggttctcacactggttccctgcacaatgaaggagctcatacctgcttttctggcttctcagaccctgaggttttcaccgaaactagacaaggggaacctagggtcagcctggaggcagggtgagcttggcgcctgcagtgcccaggccctgggtggtgcggctccggccaggccctgtttagcttcctctcccacccccacagagggggtgctgtcggcaccgattgctcattttcccctttgctttctcttcagctcgtaaaactcaagtcctgacaatgccttgatgacttccagttggtaataaaagggagatgaagataaggacaggaatttcggggaaatttctttccagttccttactaatgtgacatttagatctctagtactgtgcttctggcatcagtgccaaggcctttcatgttggagaatggaggccggggtcaccaggttgtgcctttatttcatgttgctggctctgatgagctgatgctctgctgattagcaaacgctgagccatctgcgcttcgcagaggcacgttccagccaacccggccctccctgcccacttcccaggatgctttgccttgtgggctcacctgtcttctagctcctgatctgtatctccacctccatccagttccggggctccttatcagcactgttcccagaactgtccatcacgatggcaacgttctctctgggcgctgtccaacatgggagctcgcctctgtgttgtcactcatgctcattgaacatggatttgtgtcctttaccatcaggactggatacccctcctggtcctttctgcctggggtcttagcacagctcagaaggaacctcaccattccctctctccatctagggaattagaagatgacaggggcacagttctctggctcacccccagcccagtaaactcctggacatgcttcaaggcccagctcagatgttgcctcctcagtgaaataatttataaacccacccttctttgtcctgccttctccctcttccctactcactggagagttaacaggtgatggttaagctctgggttcaaatctcacaaggccacacacttagctatgtgacttcaggcaagttaattaaccactctgtgcctctcgtttcctcatttgtaaaatggaaatagtaaaagtgcctaccagcatggcagttgaagttaaaagaaataatatatgtgaacacttggaagggcgcctgacacatagtaaactctcagtaaatactagctgcttttagtggctattcttaacacaccctcttcagtgctctggtttcactatgttttatgggtccctgagatcgaaagtgtccacaccgactcatggtcagctgtaacctgtgcctcgtgtggggaccaggctgccatgtgtagtctggacagtgtaggaggtggcagagctcaggcctgttctgccctccagcccagagagccacgtcgttagatgtcatgggagactgtggtgccccgggaatctcacgaatttgcccacggtactcagtgtctgtccaatgctatgggagtccaggactctaggagccagttaaggtgctgggtggccacaggtccctggccaaggtccaggcctctcccctgccacctgatcctcgagaggccatcacgagggttgtacttcaagaaccactatccttgagctacctaggagctgcagaatgtgcactctgcagggcttagggcctgcagacaagatagatgcagggtgtctagttaaattcgaacttcagataaacaacaaataattttttcaaataattgtgttctattcggtccctatttgggacatatttgtactaaaaagtattcatttatctgaaattcagattcgactgggcatctggtgcttttgtttgctaaatccaagagcaaatttgttctagctacttctcaaccccaccttcagagaggaagccttgatggtactgtaacatcatgctgtaagaaggggatcccttgaattgtaaatggcactctgataagatgaggtatggggattgtattggtttcctgttgctgctgtcataaattaccacaaacttagtggcttcaaacaacacagatgcattatcttacagttctggaggtcacaagtctgaaagttagggcatcagcaggactgcattccttactgcggagttctagagaaaaatccattttcctgcctccttcagcctccagagacacgccacattctttggctagtggtctgcttccatctccaaggccagtgggggcttatcaagtctttctcacatcacatgactctgtttcttctgcctccctcttctacatttaagggacccttgtgattacacaggggcccacctagaaaagccaaaataatctccttattttaaaatcagctaatcagtggctttaatcccatctgcgatcttaattcctgtcgccatgtaacacaaggtattcccaggttctgtgggttaggacgtgggtgtctttcctaccacagggcagtttctagtgttgcctcttctccctgcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtggtaagggaggggtttggctgctcgccaattgcaaggtgattcctggggtagcagagcctcacgaattgaccttggggagggcgtgagcctggtgttctggacaatccttgcaaaagctccaggctcccagggctcaaaaaatcacaactgatagtatttctagaacagtggcccagggacccagaagtcactatgaggttcaccattaggtatgtggctgtggcatgtttgtgtccactctaaatgtggggataatcccctttacctcctctaacagagtggtaaaggaaggaggaggcctggtttgactccctgacctgctatttcctagccaggtgatcatggtaagatattgaaccttttctggtcccagtactcatctataaaacaaatataatactttacagagtggtaggaattatacaagaaaagtatacgcaaaacatttcataaattttaataaatgatggccccatgcttcttcctctggaaatggtctcaacctcaatggttggtgtttctagagagaaaaaacgacagagaaagtttcatagtctcaaaaatttggaaagccctgatctagctcaaccctttgttctagaactgcatcccagacagactgcttgggacctgaaaatatctcctcctttgctagaaggataagatgagaaggaattagataaaggaggtgtagagcagaggttttcacactgcaaagtgcataaaaaccatcagagggccgggcgcagtggctcacgcctgtaatcccagcactttgggaggccgaggcgggcggatcatgaggtcaggagatagagaccatcctggctaacacggtgaaaccccgtctctactaaaaaacacacacacacaaaaattagccaggtgtggtggcgggcgcctgtaatcccagctactgaggaggctgaggccggagaatggcgtgaacccgggaggcggagcttgcagtgagccgagattgcgccactgcactccagcctgggtgacagagcaagactccgtctcaataaaaacaaacaaacaaacaaaccaaaaaaacccatcagagaagttggtaaaagatgcaagtgctaaatccccacccccaatcactgtgattcagaagaaccaggccaggcccagaatctatcctgttaccttaggcgattctgatgaagaccattgtaggccacactttcagaaacactcaaaattagaatccttcagagaaggtggcatatataatatttctagcatggaattatgttttttttcttttgcctacattttaatttctagaactgtgttgtagggaatgtcagtcactaagaacttgattgaggaactgtgttttgtctgtttcatgactgctctctcaagtcccaggaaactcactttcagcttgtcttaaaaagcaagctgaaggcttttaaaaatgaagcaacatgaaataagacaccgcagtttctggcacggtccacgcttaatccccttcaatgtgtgactttccgtggaaagttactctacgattttcccagctcgtcagggtggggccccagagtgagtatgaagggtcagagcctagggatgccaccatcagtgagagcccaggaccccagaaaaggtctcttggctcaccacactgtaggaaaaataaaaagcaatgtagtccaaatgtctctatccaaagtttcaaaaagaacttgattttagacacgctccttgacttgttttcagaatcagacagaagagtgaggcaacaaaggtcccttattccaggcagctgaataccagcacagccaggagtccagtgctggtgtttgcagagccaccagaggctccctctcaggtgtccagggcccgcatgctttgtagaatgggcagaatgagcaatgtctgtgcacctgggctttgcaggcagggcctgggtacccaggttcgtgcaatcctctcgtcaccatgaagggagcagcatcattcttcccttcttgaagcaccttggccaccagtataggtaaatttacctcccaggacatgaccattgattctgggatgtcaatgccagagatagtagggtaaatcggcacctgggtaaaactttccattggagactagaaccaaaactcaggacactggcttccaaatgtttctttatcagacaagaaagaccaagtctttccttacgtcttcacatgctgccttggcaaatgctagcattcacaaaccctgggctaccttgacctgtcacccttgcagacctcagacgggtcctgggggcttgctttctcggtttctgtatgcaggcactcaaacctgcatcaggcacctgtgaagggccgggcactgtgctgaggccaaggctccaaatgtgaaccttccaccctcactgaactcacagccagaccagagacaagcaaacaggacatttcacagcagtgcagcctagaaagggccaacaccagcagcatttgtccccccgagcggtagcttttagaagcttccccagtgattcaatgtgtcctacaaatgcctggcccccactcccagagattctgagtcagctggcctagggtgcagccttgacttcactgtgttaaaaagcttcccagataagtccaatgtccggccaagattgagaatcactgacctagagtttaatttaccacctcagtctctatagaccacgcataataatagtaccccacacacctctgagggtccaaagaactttcatttgatcacccatgagaccaccgtggtgtggagatgctttctctctcctgttctcttaacaaagctggtgagcgacagagcctgcagtggaccgggagatggcccagaggagaaagctctgccgtagtcggcctcagttaaccacggagcaccacccctacctgctctcctctcactcctgcttccgtctcggtggagaaagatccaaccgaagcaggacacatctagtcttctggtgcctttaaaatgtacttttccatttgacaaatggattacactaaaaacaaaaatttacaaaaaaaaaaaaaaaacctgaaagaaattgcaggcattaaaatgggactttgcctttattgctcctgggcccatcctatttgggtttttagaaaaacaagcctgaggcaggcccagaaaggctcagggcagaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgagaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggaggaaacaaaactaaatcaggaaggtttttgttgttgtttttggggggttttatgaaaatattcaagccacagcaaatatatttgatttatagcattagtattttttctgcctgcatctacaaaaatctttacctattaccatcaaaatatcctctgggtgaatggatttcaacaaagaagaaataaaaatgaaatagaagagaggccccttcgtgcacattgagcctactggctggattgtcacttgcctgccttgatgtcttttcagctccaggcaggcagtaggccagggcttattttcatgacagatcagatgttcttttatggatttacaaagaaagaaatactgagaagtcaaaactgaagtcacttaagacaagagcaggcccctgggaaggctgccattgaggataatgagtcctggggtcctggcctttgttcagtaaatacgcactaggcgcctacaatgtgtgcaccaatgtgtgaggcgtcaggttctctccagggtcagttggttttaagaaaggttttggcttctgatatgttttatctctacagaacagtagctcttaacctttcttatgggttaggattaccttcgagaatctgactacagctctagacctgttccctaaagaaaactaagttcacagggacacacaggatggggctcatggagcagctgaagccagaccccaggttaatagcctttacattaaaatgtttttctacctaccactaatatgcattctttagtaagcggtctcaatatacaccgattcttccttaactctgtttatgaagtattcagcatcctccctgcccccttcagcatcctccctgcccctgagcacaggatccaatggcgtgaggaccacaggcctgggcagctgctggggcatacaggcatctcttagtggctgagagactgggccctggctctatgttggctcctaacttgctgccatttaaaggaaatcttagcctcccatccgtaaaatcgagaaaataagacttgtcctacacagctcatgaaatagtaatgaaattcacattagagaagagatggaaaaacactttgaacaaaaagcattttgctcttataaaagcacagcctcttttgagaggccctttgctccccatttctccttcttcagacccccccagactaggagaaggtctgtctcatggagtgaccttttggctgcctctagattccaagctcagttttgctttcattaaccacagatactgggacggacagaaaaagacctagtttctgttgagccaaagagtctcataacttgtctgttcacatacccaagagcccaccctctagttgagacactcagttccctctcattctgggagactgcatgtctctgtgacctcctggtagagaccgtttgacatgtcccccaaccccccagtgattgagtctgaattctccactgatgacgcatttcctagcactcagggtgtcccctcctggttgccccctcaccactgaagcccgcttcctcccttttcatttgatgcttaacaactgtcagtttgcaagaaacatgcttcaaatccacattctcccagttgcctagcaacaacttccctcccggataaatgtgggtttcctgtagctcagcccaggactgaacacagcagcacacacttctgtccactgcttcaactgcttttcacctctggtctgcatgccttcaagactgcagctcatccctcccttcagaaccttccatagcctgcagaggccatgtctgccccaaaaagacacattgaacctgaggctacttatttacccttgtgttaggtatatcctcaacttagaaattaatactgtttccagattgtcttctttgaatcacagaaagtaaaacaacaaaacattcaatgcttaagacatttcatgtgcggttgggtgacatctgtttgatgaacacatttgatccaaagcatcagaaatactatgccaacaagactttttaggaggtgataaacatgtctgttctaccttaagaaaaaaatattacacagtcccaagggagagacatggttttgatcccagacaacccaagcagagacctcttagggccggaatcatcttggctgctgcctaggaccttatatcaatttcttaagcacaggatcaaggcctaaaggccccttagactgacctcagttagtagaggcagatcccttcacagccttatcttccttagaggtctagtctgaccttgaacttcggctggcagtgctgtcagttgtgatgtgtgacatggaagagttatttgttacttggaaaattaagagaacttatttggcataggaaattgtgtgtgtgtgtgtgtgtgtgtgtgtgtgtgtgtgtgtgtgtgtgtgagatgatgtttgccattttgatctgtgacttttttttccagaaatagtttctcagttccattccaactaaacttacagtctcttccggttctttgacagaaacaattcatgtgaatttgaacagataatagggaagggggaaccaaaagaagaggagagccctgggaaagttattttataatttatggcaacctcagtcaggcaactgtgaacaggtacatatggagggctccctcgggactaggcagtattcagagatgtaaggtgtgaggaccggaccctcatcatttaccattcccactaaaaagagctgggaaggaaattgtagctgtagcaccaggcacgtaactggagcttagtaactatttggtgaaggaatattattaaattattaacaagatggaaaaaagggtattaaccacacaaaaatacatctcaagctattgtttctctgttccctttcccccaaattcctagtcttgctcttatctggctgtctctctagtcactctttcttgctgactctcttcacgttcctttctccacctggaattcctgggccctccccttttactgacagacactgtcctcactctcacagtcatcagtttgtctctttacaaacctcagctcaagtgtcacttccccgtccccaggtgaaactgactgctccctccctgtaagtcaccatgatgactgctatatatagccctcatggaacctaaaacctcaacagacacagtctctttcctactctgttatagtttatttactcattaattaccacaacacgtattattgagcacctactgtgtaccatgcccagaagataaaagacaaacaaaataaaacctattcctatgcttaatgagtttacagtctagtggagagatagatacattaaaaaataacagcaaaccaaaataaaagtggtaaataaatgcactgagaaagacaggaatagctaggaggggcacctaatccctagggaaggaaagctggaagagcatggtgatgggggaagaaggctttctggagaaggtgaggtagtttgaaatgagttgactctggccagtaggggtagagtgagaatggggtgagacagggtgggttggtcattttgatccattagtcctcaaagtgataggactagtggctaaggactgcaggctttacagaagcctacaaaactatttgagatttgaagtttttttttttttttaattggctccaaaagaaaatgaaaaaactttagaattataatgaatgaatattaaatgaatatttaaggaaggtaattttattcaacttcattgttaaatttagttaaaacaagcccttgagtttcattcaacactgttttatcataccgttgatgagagaaaacaaaactgattcctggccagggccactgtcagcgtggggtttgcacatctttcccatgtctgcttgggttagctccaggtactcctgtttcccccacatccccaagatgtgcccattagtggaaacggtgtgtctgcatgattccaacgtgagtgagtgtgggtgtgggagtgagtgcccctgccatgggagggcatcctgtccaggttagattcctaccttgtgccctgagctgctgggatggaatccagccacccatgactctgaactgaaataattgggtgaataattatcttactttttaattaatctttgaaaatgtatgtatagttcacatgtatttcaatatttaatattagaagtattttagtctttattttgaagtttggtgatttattgtaaccagaaacaagctatagaaacttaattttgggccaagtgcagtggctcacacctataatcccagcattttgggaggccgaggcagacgcatcacttgaggtccggagttcaagatcagcctggccaacatggtaaaaccctgtctctactaaaaaatacaaaaattagccagatgtggtgggcacctgtagtcccagctacttgggtggctgaagcaggagaatcacttgaacccgggaggcggagcagtgagcagagatcgtgccactgcactcccacctaggcgacagtgtgacactccatctcaaaaaaaaaaaaaaatagaaaagaaagaaacttaattctggtttatatcaattagcctgtggtaaaattggtttcattatagccatttcacttagttgaagtttccaataacctgtggatgaattaagtgaggatttactatattcataaaatcttaaattccaaagcctgtttgcagttcaggtttttccactttacaaacacttctaagtattcacaatgattgcttaaaattcataccagataaatcattaaataagttgttcaaagtcaaataatttcataagtaaaaattaggagcttttagaaaactatacctacatagacctagacctatagatagacagagatctgaatagatatggacacagatgctttccaaagtgttcatgtgatgtgtggtggagtttcaagaccagagtgtgcctggggcctgcagaagtaaaggagaggggatggagagaagattgtccacatggccatgggcaatctcccacccacactcaagtgaggaagacaggaaacaaattcagaaagaagagaaaataatcaaaactgatgggagcttgtgactgatttacttatgcgcagcctccctggagacatgagtgtggctgttccttaggttgtgcctctgggctcctaccccctcttagatgccttcctattatctaggacctggttgctttttgtctgcatagcttctttggattccagtctttgatgccagcttcctcctaaagtagcctttcagatgtcccttggttaccctctgctatctaagggctcatcctaccccacactcattcccagcaccaatttctggatctccaggctggagatttagacaatgggatgggaagaacccatgatgggtcccagacagaaagtggtgccagccacagaaagggcacacaggcacagaagttggtttggggtaagacgatgtggtcagttcagaacacgctggatctaggcagatgcccagcagacagttggatatgtaagtctgaagctctggggagaggtctaggttggaggtacagatttagaagtcatcaacaaaaaggtagcagattaaatgataaaggaaatgagactatccggggagtgtgcagagtgagaggagcaagggaggcccttgggaacctcagcacttcaggggaaggtagaggtacagttgctggtgggaaaggcagagaagtagcaaagcaaaccaggcaaaagcagtgtcacagacgaccagggaggaaaaggacatgatcaaaatgttgagaaaagcagagaggtttgaaaatacaagaagcaaaaatgtccactagacttaaaaaccaggagaaaactggggggttcttgataaagcatcttagtaggatggtgagggtagaagccagggaagtgttggtgaggaagtgaagtcactgattacggactatgcttaaaagaatgtgggaatgaagggtggaagagagaaattagactgtagctagggagacataagcgatcagaggtagattctttctctcctgtgggagaatcttgcacgtatacacagcatgacgacagtgatggaagggctggcgaagcctcagggagactcttggaggtaaaccccatgaagggaggactttgtttcattcactgccgtgtccccagcacctggcacaatagcagacactcaatacatatttgtcaaatgtgggattttatcatttagaaactgcacctggctgtgagtaacaaaagtcagagaaaccgtgggtttcatttttctccccaggcagagtctggagctgggtcctccaagaggggtttggagcaccacaggtttcctcaagacccccaggctgccctgtgtttccctccttcatccccagcatatgcctgtcatctggtgacctccaaacacctgtgctgcctcctccagcacatccatgttgcaggcagggaccaggcaaagggcagaggggcctacttcaaaagaccatttccagaaaccccatcctatgacttctcctggtgtcttggttaccattgtgccataggctcaccctgtatgcatgggaggctgggccaggcattatgacttttagcaatattgcatagataagcatcaatctttgtcactgtgacgaagcctagtcactcagtgctaggcaaggttaatggaatgggttggtgtgtgcattattcttgaggtctttcttatgcttcatgttatacatttattaggacgtttaggcaacagggggataaaaatgaagaggagatgcatgctatgatctgaatgtttgcatcctccccaaaattcatatgttgaaatcttcatccccaagatgatggcattaggaggtggggcctttcggaggcaattaggtcatgactgggattagtgcccttgtaaaaccccagaaagccagcttgccgcttccaccatatgaagacacagagagaagatgccatctacgaatcaggaaatgagcccgcaccatgcaataaacctgctggagccttgatcttagacttcccagctgccagatctgtgggaaatagatttctgttgtttacccagcttatggtattttgttgtagcagccagagtgaactaagacagtgctgatctcgtattcttggagggaacccttagtctttagggaaagcaaagccaccatttggggcagggtgttctccaagtgctgccacatatgctgatgtggttaaactgcaaactatggtaaaaatgtggaggtctgtggaattgtcaatcaggaaaaagatataaaaagaagttaaagtcttcgtgcttctggaaggatatgtgccaaattgttaacattgattatccttgggtagagatgtggggaagtttgcagagacagttttgccttgtactttatataagtaaacagctactacttcgttgtcttaaaaaaaaaaaacagcctatgtgctcttcatgtgactcagaactacctaggcaatacgattaattgaattagtaaaattgagtgattatgaattttcaggaagtcattaatttaccacttctttattacatccacttctaacaggacttcaatataggggaatttgacttcaagataaaaagaccaaatttatttacccttttaaaaaaagacaacttaaaagcagacttgtcttacagaaccttccttagttggacatcgatgagtgtacagaaaatgcaatggataaaaagcttggtgatacaaagataaaaagtggggtcctgtccttaatgaacataccatttcatggagtatcaggtgtataaacaattataatcaatctgcttgttattctgataagatcatttactcacacatcaaatactgagtgcccaccacatgcccagcatacctagaagtcatccagtatgatttctgtctacatggagcatagagtcttacaggggagatagatgacaagtaaacaccagaataattaccaatggtgaagagcacaaggaaggaaacagaactcctaaagagagcgtggctgggcaggggtgagcaagaggcatagaaaaaggggcatctaaatctacttgggaggaagctgtttctcacataggtcatcatgttaggaatgagacttgagggatgagtagaagtttgccaggcaaagaaggaatgggggggaatagagagcagagctaggggcaggagacagctgacgtgtgagcagacataaaaagaagtccactgtggcagcagagaagcaggagagaaggcaagtgagggagccaggcaccagctcacagaggtcatgtgtgtcaaaacgtagtaatggccttctcttctggagacagtagggagccatggaagatgtttgagcagggaaagcgacatgactggattggcctgttgggtaactcagaccacaatgcattggaagggagggggctagaggcaaggggactggcaagaaggccagtcctttttctatgcctattttgatgaaatattctagaagggaagtgaacaaaggtagtcctagagaggaagaacaaaacagataggatacttccttagtatttgctcattcgacaatttatttttgcatatacactaaaaccttttttattattaaaacgttttattgtaggaaaaaagtatgaaagtagagtgaataataaaatgagctcccatggatctatcacccagcttcaactattatcaatatttggctgttcttgttttaactgttctccacctttttttcctgaagtttttttgaagcaaatcacagacaacatatcatttcaccatatgtacttccctctgtatctctaacatgtaagaacttgttttaacaaaatcaccatgctatgatcatacccaacaaaatttatcataatgtcttaataatacctaatacccatttcatgtccactttcccccaattgctacagctggtttgttcagatcagaatcaaaatccacctgtggccattttactgctatgtctctcaggtctcttttcatctctaataatctcaggggagacaggagggaggacgggcaggacttggggctaacttgcttatcgacacacagttttgcctacttgcttcctcccttcacacccactcttcttctcagccccacccttgtatggaaaaaacagaaattaaagtgctttgcccagcacccactgaagctatttcgaaggagtttgaagagtactcccggcaagacaaatgcctcggtccagtgctcaggtcaaagaggggagacgcttctcagtgatgtggtgtcaatagcagcttagttgttctttcctctggaaaattctacccatctgctttgtaactcccatacctaacaaggccttttatttcacaattagaaaataagcctgaaatatgaatgctgcctgagtgtacctacatttattctagagtttcagggtcaaaaagaatacaaggacctctgcatctacagccaagaggagaggggcaaagacacacagctacaaatgagaacctggctggtcaaagcctaactccacctgtttgtcagcactgatgcaagttaggtcagcccaatgatcatttaggagaactgtgctggcaaataaaaagcagaggcttttggtccccagatacttggatgagaattacaagtccagctggttaaaaggcacatgcccagtgctcacttcacacctactcaggaagcacacttgagttggaaaaccactgtctttacacttagaactcagtcctacatgactcctctaggatcagtgattccatcagttttgaaacatgaagcatgaagtcaaacaggacatgaccttggtttccagaaaaccagatgttcacatcagtctctggagcttggaggcagcacacctggggacttccacatcccctgccgaggtggcaaaagcaggagcagtggtgagttcacatgggctggggtttcctgaacactgctggcaattggagaatctgcaagggaacttctccgactcctaccagcagctgctttaaaataaaggtgatgtagctggtcaaatcctccatgagagagcagtgttgaatggaggaagagacacaacctgtctgaaaatggcacaaaggaagaaagatgtaaacaatgacgagaagactgcagtgtctacaaagctccgaggtgaacagatgggcaccccaggcccgcagcacttccttcagtctctgccagctgcactctgttttccttcctccaggaatcttgtttggtgtcactaaaacagcaattagaatcactttgaaatagtgatagtatttaatataactatgaaactatctgtgattgacaagtgcagcaaggagtcttggaatgagagcctttattttttcaattaaataaaagagttttttgtttctaaaagtaatcttgcagaaaagatcctgcgatcagaaagaaggagggggggagttttcaaacatataggagatcagactgtgcctatgtgtgtatatacctacaaacatatatatatttaaaaaattgttttactgtcaattacagcttcccacactcctagacagccgttctcaaggtatcaatctgagatcttggggaggaatattatctgatatgtcaccaagaattcaagaggtgagtagcctgatggtagtaattataatttcattatgtctttccaccatttaccccacttatgtcaaataatttaattgtatttcaaacctgttcaaggaaaagtacatttgatctttccatctagcaatttcaaagcacctgttcacatcccaaattatctgtgctcttaagtaagaggcagaaagaaaggaaccacccttctgatttcacatcaaaaaagaaatgccactggcaataagcaacttgcctggtgtggcataaatcatcagaagacttacagttgaatctaagtcttttcagtactgaggtggttcattattctgttacagtcttaaaattcacataaatatatactgccaataataatagcatacacctttatagcttacaggcactcttcttctaagtgttttacctatgttggcttatttcatcataaagaaaacaatggacttttgtgttgttttgtaaaaagatgcgcacattttaattaacatctgattgcacaagtctcctcccatatagaaatggattcttccacgcaatagataagaggtgctggggatatgatgatgaacacacagatttggtcatgaccctgtgggaaagagagatgggaaaaaaacaattctcttcaagtgtgatgagtgttacgaaagggagggaaaagttgaaacaggtttttttccaaacttttctccctccattattcgcagctgacttgggctccaccaacctggaaaactgcatggttggaatctgtctttataaaacgcatctcaacctgggccgagtatgcacactgatgtgggaaagttagagaagagcccattgtactaatgctcacctgctacagtgggagtctctgttaaacagtcttttcttcatagcattaaaaaaatttatatcactacaataaggttgaaattgatagagaatgtacaaacaatccccaaagtatatcaacactcttagttctgagtagaagttccagaaggcttcttgactgtctagatagcaagtctaatcatttgtgaactaagttaaagcagaaggcccagtttatatgaattggtattacaccatttgacctgagaacagccccttcatctctgagtgctttgactaaatgagcaacataataatagtaataaccccttacaagatgtcataagactcactgttgttgaagcaatttgagattttgactttattgaagcatagatggtgattataggcatgactcactgtgtggattctccctgggctcatcagtttcagagggcaagtgttggcatgtggacaaagagagggatgacacgtaaacatggcttattgcaatggggaaatattttcagtctcactgattgaatcctaatggttttataaattccccagtaccactgaaagcaaagcaagtaatcaggtgtgttttaggaataaaagcagcattattttaatttcgtattttcccctaaagcaaagccaaatggcattatgggagccaagctactggcagctccaccagccttctcctgagttctcggcattacagatctaccctcaaaggatgaggccagcaagcaccacagggtgcccacatggagaagagaaggccaccaacctcctcttagctggcacagaattgaaaaagtgtttttccaggaatggatacttcatctgttctgtatttgctagaattttaaaacgcacacacagacacacacaggcgtgcacacacacacgcacacacacacgagaaaaccacaaaccacacatttcaaggaaatggaagaattcattggtaaaattaagctaataagattattttccaaatataagaaactaaattttagactatttagccaaagaaatttgctctgatcttgcttttctacaacagaatcattccccaatcattttatttccctctttttctccccagtatccccatcttggtgggacaacagaacccaagaactggcttaacagtaaaatattttctgcatttgcccaaggacacattcccaacgaattcaaataaaggagactagaagaagagaggctatactacagtgctctaggggtcactctgtgatttgttgttgttgttgttgttgttttgagacggagtattgctcagtcgcccaggctggagtgcagtggcacgatgtctactcactgtaagctctgccccccaggttcacgccattctcctgcctcagcctcccgaatagctgggagtacaggggcccgccaccatgtccggctaatttttttgtatttttaatagagacggggtttcaccatgttcgccaggatggtctcgatctcctgacctcgtgatccgcccgcctcggcctcccaaagtgctaggattacaggcatgagccactgcgcccggccactctgtgattttctttaaggctcatcctagtattctcctagtccctaagtagatggcagtaggttttgttttttgtttttcgcagctggattaaggattgctgagaatatatggatgttttcttttaaatgtggaagtcaaaccaaacgttggagcattggcctcacagcagattatgactctagctgccttaaaataacctgaagactttgccttgccctagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaagtaagttttctgagtcctgcttataaattggcctctcatgttggttaagttgatggtttaacacttctaggtgaaaccaaacctggggttgcatctgtcttgtcttgctgagtggccttaggtaaagagacttctcccagaaagtccacttccccttgcagaaagggggcattgcttataagcaattctggacatgaaccacagaaagaactgaggcccacttggaaagggaacagaggggccatttcccactgatgtaattgaactagggctaagttcaagaggaagagaatgatccgcaaggaagcaacccagagttccaggtgaagctcaggtcagaagggccctggcaagtaaacacggctgtgggatgcttttacaaacacaatatcgtgaaaatctatgtgtgtagtactgaattacattccaaatggcaaattcctggcaaatcatcttccccacctttcactatttttttttttttggtcttctatggggtaaaggaggatggggtggggaagaaatgtaactggctgcccctctagttaaaaactgaaaagaggcagcaagggacatgccaaaagtagttggactctaagatagctacacacaacaaagcagctaagcagctaattgaagggaaattactgaggctcaagctgagattccaagcgggggccttgtttggcctctcagtccctttcatctgagaaaggcctcagttcctagcagtaatcagaggcaggcttctcagcctccttctcctaaagcagaataaaccacagggcaagtcgcatcctttgtttctctgatgaggccattactgagagtcactgtggcattttgctactaatgatgagcttgttattggtggggtacagcctattaatttaggttattcatcaaatcctccagcatggagttgaatgagacatgtgatgtggatacactaatgactatattgagttacaagcaatggggagtttctgtaaaatctgtcccttgtctcctggcagcatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgtaagatcccagctgggcttgccttgtgtaccctggacctcccagaagtgtgtgtgtgtgtgtgtgtgtgtgagagagatgtgccttcctggtagcacatctcatgtttgttttttgctaagtggactcttgcgtttcctcccccatccacagtcatcactggaatgctttgcttcagtgcccctgcctgggccctcccctctctactgcagcctacaatgaggttttctttcccattgcttgaattatatccctaatggaagggttcacaattctctgaatcctggctactcagataaagacagggaggaagggaggaagggtattttctcccagggggtccaaatctagctttaacgagggaggttctgagaaaataatatcatcaatattacatggacttctgagatactaagaaattagattctgtcagcccaggaagttgggagatggtgaattgttctgggaaatagcaatagactgagaaaataaaaacacttccttgaaaagcctttccctaacactaagtgataggggcagaaaagacacaaccaaaagttctctctcacttttctctctgttcgtgtctctgtcttgatctctgtctggttttaggccaactcaacttttgaagaactggaacacgttaggaagttggtcaaagcctgggaagaagtagggccccagatctggtacttctttgacaacagcacacagatgaacatgatcagagtaaggggggttggaggatggggaggggaggggaggaggaagcggtgggggcaagaaagttccacttgtttccttttcccaggaaagagttaatcgctattggagttagatcaaaatacaacaagcaggccccaaaggccttcattccaagcagtcaccaagtggggtcactgactttggatgagaaatatgtttcttgaattctgggagaagtctaaaagctgccacaagaccagtggcttcctggagtttcctacttttatgaattcactcaagggcctcaaattcaaagaggcatctccccaaggggccagctctgtaactccaaagatggtggaatgtgtttgtctggtctcattttcagctttgcaaaatgaagacaagagttctatatatcagggacactcaaaagaaaacaaaaatatccataagcaaaagaaagctttttatacaccatattcaatgacccccatctggcccctcctttgcccctacacatcttccctctattctagagacccatggacttggggaaatgggatatagataggtatgtttcatagtggaacaagctcaccagctcttcagggagccttagcatctctatcctcaatcactaaaaattagaaatggctgaagaacaagaccaaagatcctatggaatttctaagcagagcagtgactgtatttcttcttcccaaggataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggaggtaaggggctgcaagccccacagtgggccccttgaagatagccccatgagtggggccagagctcccttagcaagtcaagtggtcttgaatttaagctttcattttccccactgaagaaacaagaatccctacatcccctgtacagttctcattctctaacagcttatccatacttaaaacttatctatgctgaaaacggtttcctcttcacatctcctacttctcatgctgggcacctcctcctgtagccccctttaagcatctgtgtctgtcctcaaccctcttctgtctgacattgcttgagtggccatctatggccagtgtcccctcaaccccacagtccattgcttgctggacactcctgccctcaagttctacaagcacatcagcctcaacatgtcccctccaaaaactgtatgttctccttgcccatagaacatatccttctcctatatttcctatcctaattaacgtcctcagcatttgcccgaattctcaagtgagggatttcagggtcatccctaattttccttcttcaccctccacacagtagctgtcacttactgagtgttactttatgccaagtactgtgccaactgcttttacacacatatgcttcatttaattctcacagctccatgaggcttgcaccattatcattgccaatttgcagatgagaagccagggcttaaagaggttaaataagatcccacgcatgaccattaagaggagcgaacaggatccagctctgggggtgcctgagttcagagcctgcctttctgatttctcttaccaagctttgtctcctctccctcctaaatatctctcaactctgcctcttgcattccaggctctctgaggactagaggccttgtcatctctgcgccagcccattccaagggcttccttcctggaatccagggtccagcctctgttggcccaggcatttctctacactggcaccagagttacattccgcacacctgcttacgttgctctctcacttaaaatcttaatgactcgacccccaaataacacaggtcccttccaaatctgtcctaccccaccttcccagcccttgctcaactctgctacctggccctttcacgcctacaggcattcccattccatgacctcttgggattctaccctttgcaaatgctgttttcattgcccatttattagagcgcttttggtcacaagctttttgcttaaccaaaaagaaagcatttattggtggacataaataatgaagttcaggaggatccaagagttggaagccaccatgagacccctgtgtccttccacctcacttttctactcgcctctgctcagcttcatctctggccaggccctctcctctgctgatgccctagctgcttacagcccttagcagtcatctatacaccaaaaatccctttcccatagcagaagcaatgctcctagagagtttccctgttggtctggctcctgtacccacccctgtgtactctgattgggaggcctgggtcagctgcccaccatggggccatttctatgagcaggattactgtgaagtggaggaagatgtttccccaaaagaagaaacacaaggtagaaaagtgtatgtccaccaatgcctgaaatgactgtccctttcctcatctgctgagcttctactcattcattctttgagactcagcactcagctcttaaatgtcacttctgctttgatggaggtttagtcattcactcctctgtgcttcctggccctctcttcacacctctctcagacccctctcccagatagattagagttggctgttgacatgtccatctctggctgggcagctaaactggagttatttagaatcagggagcacatgtcagtcattttcaaattctcaacctcatactcccagtaaatgactccatctaagggtggaccactcttgcccatgggccaggtctgggtctgtgtcatctagaactgttggaaggtaggggcttctgtgagcagtaggagagggaataaactcgagggccctcgggagcatgccctcttgtctcagacttgtgagtcctgaggataacaaactagtgaagaaaagcctcgttctatctgtcacctggtgctcttgaggactttctgttgccctggtgccaccacaattttccagagtgtgtgaccctcgctctccaaactctggaagtggcagccgaggctccccagtggcctttcagaaggtgccagtcatgacagcagcaccaaactgcaggcaactactaagcgatcaccaacttgtctgaagataagaatgaccttgaatgcattttataaaacaggattttttttttaatttttagattttctttctttattttaccttaagttctgggatacaagtgcagaatgtgtaggtttgttacataggtatatgtgtgccatggtggtttgctgcacttgtcaacccatcatctaggttttaagccccacatgcattagctatttgtcctaatgctctccctcgcctcgcccctaccccaccccaacaggctccggtgtgtgatgttcccctccctgtgtccatgtgttctcattgttcagcttccacttacaagtgagaacatgtggtgtttagttttctgttcctgtgttagtttgctgaggatgatggcttccagcttcttccatgtccctgcaaaggacatgatctcattcctttttatggctgcatagtattctatggtgtatatgtaccatattttccttatccagcctatcactgatgggcatttggattggttccatgtctttgcaattgtaaacatacatgtgcatgtatttttatagtagaatgatttatattcctttggttatatacccagtaatgggattgcctggtcaaattgtatttctggttctagatccttgaggaatcacactatcttccacaatggttgaactaatttacattcccaccaacagtgtaaaagccttcctatttctcaacagcctcaccagcatctattgtttcttgacattttaataatcaccattctgactggcatgagatgatagatacccatttgtcagatgggtagattacaaaaattttctctcattctgcaggttgcctgttcacgctaatgatagtttcttttgctgtgcagaagctctttagcctaattagatccatttttcaattttggcttttgttgcaattgcttttggtgttttagtcatgaagtctttgcccatgcgtatgtcctgagtggtattgcctaggctttcttctagttttcatgattttagattttacatttaagtctttaatccagcttgagttaatttttgtataaggtgtaaggaagggatccagtttaagttttctacatatggctagccagttttcccaacaccatttattaaatagggaatcctttccccattgcttgtgtgtgtcaggtttggcaaagatcaggtggttgtagatgtgtggtgctatttctgaagcctctgttctgttccattggtctatgtgtctgtttacaaaacagattcttaagcatcaacccagatcgactggctcagaatttccagggaagaggcctggttatctgcatgtttacagacctattagatttgtgggacctgcagttcccttgtacagttagttactcaattaacatctccctcctctcatggtgcctctacctgctaagcccttattcccagccaggcccaccaccatccacccactgctgttataacataagcaggacctgtgcgagggggtgtggacggaggagagaggctctgttgcttcatttgtgcagcatggagttcagtggttctcacaatgtttttgcaaagtatataaagaatactccttgtctacttgacattcgtatcgtgacataaatgtcttgttttccagaaggattattttttccaagcagcttgttcctaatgcagccccaggcaccaaacagatacttaaaatatattaattgcttaaatggttaagaattcagtctctggacccacactgcctgggttcaaattcctattatctgtgcccagtttccaagtctataaaatagggatattaatagcacttacctaataggctcgttatgagaattaaatgagctaattcatgcaaagcactgacatatagtaagcacttaataaatattagctttttaacaaaatacaagccaaaaaacactgcttaggagaggaaatgatgttagtgcctcctgtaaataggcccagcctccaagctggtgctcctctaggaatcacaacgctgcaaatcacatcctccggggccgccaggacttcacgagggcctctgagcagaggggtatgatgggagcagaagcccagcagctgtgatgatgtggtttctgatcttcctgcccttggggtgggggaggaggaaagcaaggggcaatgaacagaaaggagaagatagcggggaggaaatgtgtgaggaagaaacacatcactgtggcttgtcctggatttttctgcttctgttctcgtgttttgggaagtctggaggagacttgaaaatcattcatgtccccaccctgaggatggcttagtagcagagaggccatgaaaactctttgctgatggctctgaaagcaaggatgttgcttcactgggctgctgaaggcctgcctgggggttctgagcagagagtacaggcccctcccaggagggcggcctaaccaccatgctggcatttctgtggaccatggtctgctgtctcagaccccctccacaatagggtctgcaatctcattcaccccataaatacattctgtctttcctctgatcccctcccattagcagggggaaataaatggaagtcagacggcccagttagaaggcaggcagtggagtaggaaaatagatgatggtggtttggggagcctcacatcactcatggggagacattcattcccatgggccttccaatcacccttttctccaaatctaaggacacaggacaaatgggtcctcatacaggcaaatatcttaaactggtatgtgtattcatttatagttctaatttatatgtgtctttattcacatatattttgcttctggagaaaagctcaattagaaaaattaatacattattcttcttattgcccttcagctaaaacaagcatacacacccctcccctttggattttttgtttagcaaaaggttaggcctggcacagatgaaatactattcagagttcacagtgtattttcatttcataatatatttgattttcaggtcttgaatttcacatcaggaagctgatataggaagctgaattcagccagattttaatacgaaaatacctctgatcaaggcataaaattgtactttaaccagtaaccactgtatttctctaagctgtgaaaaaacatgcattcattaactgctttttcctctgctgtcaacacagtcaatacatgtgcataactccttattgtctacatggtgattatcttgctgatgaattctcaaaggccagagatttggactattttttctctgtaaccttgcatgttcctggccacatgccaccaccacccaaacagaatgtacgcagggaatgtatttttcaggataacctaagaaaaaataggattaagaagataaagctgctgatcatgtaatgtactttagactcagatatataaatatttgtgaattatctgtcctatttctttcttctattaattcattgactctagatgtgcattggaaggctagggagaaatcaggggatcgtgagaaagagcacagaagtctgcatcacacaaacaatattatttcaagagccatgaactagatcctaagcaactcataggcaatgacctcatttcatacctctagtctctaagaaacatataactggcctgaggaaggaaaatgtgggcaaggggtagaccggggtcatgggtggaggtccaaatagtaatcaatggagctcatagggtggactgatattgaagctgctatgagccagccacatgctgggcactgttacatgtcatctcatgcaatactcccaattacctgcctagtaagcataattgtcattttatagaattaaaaacagactcaaagaggttgacagtctaatgtaacacaacagctaaatgggggatctggaattataatccagagctgcctggctctgatgagaaagctctttctgctgtcatatgcagcccacattaatagggggctcagaaagtattctctggataaattatataatgaatccaatgaaggaagacattattttataatatgcagcataataggcactattatgattggattttcctgcttgaaagtagctagattagagtaggaaaccaaaaagatgtgaattcattcagtcattcatgcatttgcatggattgagctacctacatttgaataaatgctgttaatccctgattccttggaagctcacattggagagataagcatgtcattaaataatgccataatagtggtatctcagaggactagcagaacataattcaatctgacagagtagaaacagattgtacaaatccaattcaaaacatcataaatcctctaagcactgtcaattcttcctccaaattatctctgaaattcctccttctttcccatttatggcctccatttacagaagcgtgtactgtctctcttagctgtttgccaggccgccagtctcttgctgttcagctctcaactgcttccagcaagatctttctaaaatcccaggcttgccaagacttagcgcccacagctccacagtgactcctcattgctgttaaggtaaaggccttcccagtctagcccttcatgcttcttccatgttctatgggactgccccaggcttcccacctggtaccactgagcctttccatccttcccccactcgactgccaggtcaacacccacacccacgcttcaggactcaggtcctatgtttcgggccttcttctgtgcaccattcccttccctgtagcccttgatcatgatttgtttatacgcctccgcaccttcatggccctgaacccctcaagggccgaaactgccttacttttctttttgacttcccaacttaccttagtggagctgtagtcacatagaatagacgctcataaatgcttctctgggctgtaaaggttgaattttccagctaagcaaggaagaaagacaatttcaggcaggaggaagggcataagcaaagtgcagagatgtgaagctcaagagaaatggatgggctgggcagaggtgtggctgcagcatcaggggagaagaagtagtgcctggagtcagcaggcacggcttgcaaaagcttcacctataggtgaaaggacaccatctcttgcaccaataggctctgtgattggaggcaactttgctgttttactgccagaaaactgaggatgataacccaaactgcagttcaagtggcattcactggtgtggctgaaatgggtgtttgtggccagaatgtggtctgattggtcagtgcccagctctgttgattagcagatgttttgaatatagtagcatccatgtgcccaagttgttgggatgattcaacaagaaactttaagagctcaagtgccctgcagttgtcagccaggtgattctcttcctttggacccagttagacgcaggcattacctcgtggctttgccccagtgtgaatctttgtcctccaacttgatctttttatttgtttcattattgtatttaagttgtttattttagagacagacattttttaacagctgtgcatttcctgtccctttgttttccagtcgtcatgtgtttccttactctctgtgggtgaacgtttcagatgtctgtttgcggtgcccagcgtgcaagataaaatttattgcagtgccttcggcctctaactcaccattccaaccaattcagatagcccaaggctgttttatccagtggatttttccatgtagtgggaaataaatcttgaatgttactgtttagattagccaggaaactcattctgggatgtttgcccacatccattggcatttctcaaaaggaaccccaggtgtctaccttgacaccagcagggccacttgagccctccgctggcattcatcgcccgctttgttctcagcctgagtttaggagttacagatgtgagaggcgggattatacagccaacatctctaagcgggcagtggctcccttaccctcgaagacctcactcctagcacgtcctggatgtattcgtcaaaatatgtcctcttatgccacgtcagcacagggttgctccccactttgatcatcaagtttaaacaaaaggaaagattttctttctttctctgcctctactggacatcatttcccacctaacagataatttaatgtatctgttactgaatgtgtttgaattacagacagagaggtcacagttaaagaaggaagcctgctgctactgcagcttgtcctcccaaggaggtgtttgatttagctgtgtaaacaaatgactgcattctccagaggtcctgaacacagctgcctgcgctggagagggctcaaacctcttccgccagggtgaactctgcttcctggtgagtgccagcaaaacaaccaacaaagagctgtaggacttgtgtggacttcaaatggtggtggtcctgccacttgggctcagccacagcagttaggaaactaaaggggaggaggaaagccctttccttgctttattgtcattggctgtcatagggcattacaatggttctctttgagattctgagctccggctataacatttgcccagaatctgcctctgaggccttaagacactgtgtttttattcagcaaagatgccctttgactccttttcccactagtggtgctaggtttgagcaccttacactggccccttacaatagccagttcttgtctacctacattcttccctaacattcatgattgcatagttactcttagtgtagaagcagacagcttttacacatagactccatggccgtagcctcatagaacctactatattctaacttgcaagctaatcagaccaaatatatcaaaatcaaaaacctctgctgagagtttattcattcatctctgtctcccaaacgtacttatgtacatacgtgcactaatatacatgtccattagccaagattttgatttcagggatcaaagcaagtaccaatagggaatgaggtcacttgctgcatggcaggtggcttccccatgagaatgcaaggccacctcatgactcatacttcagagggtgacccaggaacttctgattcatgtccaaagcagcttctacaattgctctaccttgatctagggaagatgtggggaggatgacattcgggattagctttataaggccttcctgtgggcagagttgtctgactttcacctagtgatcaacaagcagctagcaagcatcagtgtgtgaggccccacgccctctcagctcccctactgcccacctgggacatgggctttggcatctgtccatagcattgttctaaccaaatgaggtgttatggatcagctcaggatgggatatgttcccagacatattatttaaagaaaatagctcccttcctcccctgataaacagctgccatggctaaaaggtaacctggctggggcttaaaagtctgttgactttcaagatattttgcaaaaacagtcataaaaatggtatttatcagatcctaactatttgtgagacggtttggtataccatagtggttaaaaacacaggctctttccagaggaggtttactttgcttagtcgtgtctcctaagtgaacttggacctcataaggttgttgtgagaatgaaatgggtgaatatgagtaaagtccttggaccagttttggccgtatagtaagccttcagcaagcatctgcttttattcctacagggaggcaattgtaagcccttcacaaacagcgtctaatgtgatccttagaacaaacctatgagatagggcatatctcaattttgtaggtagggaaacagaagccacacaattaggaaatggcaacagatctgttagactcttaaacactatgctacaccaatttgcaaggcaaggaagacaaagcacctttgaaaatgggtcagatgttttagggtaaatgaacgtttgagaatcttttaagttttttttcccccagagattatcaaggtatcattgtagggggatgcatcaggaaacatgactatgaatcagctgcctgataaaccagccaggatggagcccacgtcatcacagcagtcagcaatgccactgaaaaacatcagctgcttattcccgtatagatttccccttaagacatgaaaagggagttcaaagagaatgggccagatatctctgagagtcatattactaaaatatatttatttttactagcttttttgttttaagaggtatactgtcattagcactgtagcaaaaattcacgttttattaatttctcctagtttatcatgtgattctagggtaggatgcagagttatattcaaaatacacaaatcaactcaactcagtaaacatatatcgaggccctatcatgacaaaatgctattctagagaccacggcgaacaagccacggccccagcctcaaagaatgtactatctttggaactgtgctggccaatacagtaaccagcagccacgcagggctatttaaatttaaattaattaaaagtaaaaacacaatgcctcagatgcattagccacattttaagtgttcaatagatatttgtggctcctgcctgccatattggacagggcagatatagaacaattccatcactgcagaaagttctactgaacaatgctgctctggagcagaagatcttcttgttcagggatgttacacccccgcttgtggctagagtgtggcttatcctcagagcaaggataggggaaccatggcactctgcaggctcagcactgaagacacggatgcaggctctgcttctgacctagattgaccttgggcaaggccctttgctcctctgatcccaatttcttcaccagccaagtaagaacatcagaccacaagccctctagggctctgtccaaatgccccatgactgagtgaactggtagaacattctatgtgtgtgtcacaacatgaagagcaaagactttcatctccccaaataattttgtttttcgttttaggaattaaatttcagattcactctaattgccaatactaaaattctctatatgcagttctaaacttgacaaaccaataaaaaaagattatttgactacttatctttgtacaacattgaggtctccctaaagcaaatttaaatgcatattttaaaaatgtattctagcagttcagttcagaagccccctggcccaagcatcacactgtcaatcctttgtcctcaagcagcatggttgggtgggttaagtactgacaaacactgggtgtcaggcccatggtcagggactgtgctaacagtctacatattagatgccacctacccccaccctcaacagacccaaactatttatccaatagcaaaccttgcattatttctgtccagaagaaacaaacatttattgacaacttttggtgtgtgacctgtttaagtcctacatctcatttaaggactggtcaatgttaggctaggcaatgcctgtttgtgagagaatcactgcctaaagaaaattctccatttcccttagctctatggtgggtgactacacatactggtatttcttaaagaaataccaattccatttccttttaacataattattaatatctcattagcatggtgtcactgaagcctgggcccaaagaaataccaattccatatcattttaagatcattattaatatctcatcagcgtggtgtcacttaagcctgggccctttagaatttttcatgtacctgtgttcctctgcccatatcagctggaacactaatagttttcttcctttttatctagaagactgagaacattacatgggacctgcccccagggcatggaggctgaggtgggacagtttagttcaggaggcccaagaagtgttgggtgtgcagccccttgttcaaacacagcctctgaatcgccagaggcttccggtgcatactctgaggcgcaggtgggactcgggagtgagaggtttcggcgaatgaattgggattgcctacttcttcccagtgcagtggagcttggttctgtggtcaggtccttacgccctgtctgcctttctcgtttctttatttctcgggtagtagttgtggaatcaaatgacctggggtttgatacctactctaccacgcctctgggggagtcactcagactcgttgaacctaagttccggggctgccaagtgaggataagtagtaattgctgatccacctacttgacaagatagtagtgagggccctgagcgccaggctgtggatccagcctttcccacggttcctggtgtggcaggaagaactctaggcctgaaggtgaaattggggagggagtcccagctctgccactgtctctctgggtgacctcaggcaggtctcctcaaaaaaataagatactttataaagctcagtttcctcttcagtaaaatgaggattccaggtaactcacagatagtttgtggggatgaatctgttccttaaagcctgcagtacatcaataacccagtcttcctgcttgctttcccccctctccactaccagtgatcatagtctgatcccataggtgatatcccagctcaaaaccctacattagcttctgtggctgtttaaggcctgcccagaactcccctggtcttagcactgaaagcacgtgtccggggaagccctgcattggtcgttcatactactgagtcccgcagggcaaaccgtccggtcccaccctcctttctagtgctgctgtcacactcacctcccttcaccctacactcccttctgtgccttgcaattacctagggagttttttacaagatatggatgccctggccctgccactagagattctgatttaattgcttggggtagggcctggcataggtatcttttaaagctccgcagtggttctaaagcacagccacagatgggaaccactgatctattcttgtaggtccccagatacctcatgtgctgttccctgtgcctgagctgacctttcccccactttcctctcctcggctaattcctgcttatcctcctactcaggaggctcttcctccaggcagccttccctgatccctccaggaagacttagctgcgtccctccgctgggcttccccaatacactgggcttgctttcattagaacctgatccttccacattatggttgttggtttgctccaatcctctccctcattagctctcaactttctttcaggaagagatgtttatctttccttcttgtattcctagagtcgaccaggctctggcacattgcagattctcagtatgcattcagggaacaacttaatcaagacaagaccatctgacttcttgtgagttacatgctaagaaagaaatgtcgacaccaatagccctcacaatgataggaacaggaggttaaagaaaaggaaatagatgcaaatagcaatataagtgctttaacaaatctatacaggaggacaaccatcatattcaaattttcaaacattcttagttctgctcttttgtgggtaatggtttttttttttcctcttccaggagaagaaaagaggcatattatagaaattcctcctcccccagcattacttgtcacagaattgtaattggaagtgatttccctgactaagttattttggctgtctgttattttctctcttcctccttgctcttccctcagctggccatcctgtgtgtttggagagagccagaaaggttcaaggctaggaatgtttctctctctctttaaagctctttaatcgtcaggctttctgatcttcaaagcaggctgtagccagtgtgaccccactccctcgcctccccatgctggagagtaaaagcctggagtatttttgtcattttgaagacttgcatatttggacagccttggacatctggaaagtgtggtcctcactagctctgcagggataagagcacgtcagcacttccaagctctctggcgcccctacatctggacacgttgaaaaattaacaccagactctggagttaagcaaacattaagtttataggcctccttgcatttgaccatttcctgggacagcagcccttatcctgtgactttctgtgtgtagagttgagtctttgcagttggtcctcctcacactctctcaactttgtgactctctgcagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtgatgtttcaggaagggctcgctgcatttctccaaagtcagtgggaaattacatttggtagagagaaagggattgagactggactcataaatcaataaaattaagttaaataagaaaaaataagatattttataaagctcaacaaagagtccttgaatgaaagcaattacagagtcacattgtggctaatattcaaaactgagatttaaactgaggactaggaaatagaattggatccttttgaagcgtttaggagaaagattttaagagaatgagttccgagtcaccctgtggtcgggaggtgtgagtgagctatccaagcccgttcccatcctttgtccctctgtgtcttctcaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattcgtgagtctgaagttcgcgatcctcctccatgacacgctaatgggggtgctggagtgggctggggtgggctgggggtgccctcaaggcttccatgtctttagagagagccccagggaccagagccaaattggagagcatggagctctgactgaggaacctgcttctcccaagctccaggcaggcacagatgagtcagtgcagtggtgggaaagggaaaagagttgatgttgtagctggaaaagggaaggggaaaattaaagcaaggaaagtgaggctgggggaggggacaaattccccactatgtagtatgtttggtatgtggaagggttctggtcagaatgtttgcccaatgattgccacatcagcattcattttggactctgtatggccagtaggtctggttcctgggagccctggaataatgcagccccttccctaactaacatttccatgatgtatgctcaatgacaaggcagaggaatgtgttggatgagctcaggacctgcctccctggacactcccatcccaggcctgtatatctgttgaccaggaataagccaagcaagcagcctactgtttgactgaatatggatttggggggtggtagagaaagggccggggtggagggttgggaggctcatttgtcattatagatggggtcagacacactaccaaaacagcagcagagatctacaattgagttcacctaaaactcagtgtggacacaggaaaccctcttttaataactgtccaatgggttttccagcctcagctctacagaaaacttgagataacagtggccagtctgcagttagtttgggttcggacaataggcagagctgggaaatggagccaggggcgaaagcccaggtccactttaggatcaggacgggagtggctggtggggaagtgaggtgggtgtggggaggcaatagggagctgggtcatttggtatgggagagtcctctggtggctagtcccagaagtgcatgctttacgaacatatgcttctctccctagggccaccttgagtgaaaccctcccatgctggaattgggccctttcagtgacaacacacaacagttttcaatagataataatcccaagggctttactagcacatgaaacacagggaaaacgtgtaaagttcacaagaaagtcgttccagtgtatcaaatctatcctgtttgccaggtggatataccagggtctcctccacctgtgcatggctggtggtgggtccagtggctgttggataactgatgtattgatggatcattcgccttctgaaagtgccaaactgattagttattttgtgtgtctttttgtgtaactagggtttgaccttccagggcagactgtgctggggcggctgaccccttggggagccaagttattgctcttaccaccaccacttgcccttgtcagtcctccaccctcttgggtttcagtgtcagcatgtagctgtctactcagatcccatccacatcatcaagtctgcagttttttccttgcaaggccttacagggaagatctttgacatagaggatataattttattgacacattttacttgcagagcattcacccgggctaaccagaaagccagcactctgctataaacaaaaaataatgcttcagggctaacatggaatgtgttaaaagattccagcccattaaatgtccaggggaggttttcctgttttcctttccctccatctgggctttgttctcaacacattcattcaacaaacatttattctgcctctaccaggtacagagcactctactattctgcttctctccttttgctttagtttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatggtaagccaaatggagaaggcccagaaaatcttgaatactttggttcctttcccctttcctcctgttcatgtgcctggattagtcatgtggccaccaaggagagcgtgacatctagcttcccagcccttccttttagccaacgtgggagacactcaaagagacgaaatctcctgaaggagccactgtatcacagcatcctcccatctcccacttcctgcccaggggtccatggtccacacagacttcccagtcccattccgtgaccatctggagaagctgctattagcagagccctgcacagggtgatagtgtaattaaagtggtcttctctttccaaacacagaaaaaatcagttcagggagtgttttcctgggcttacaattttaactactggctagagttgaaatggggaaagccttttgccttttcagtagcagtaggggaggagatctggattatttacttatcatcatcatggtcacctcctacatggcttcaccaaaaaacattctgctgcctgaaaaagctccaacacctctctctcttttaaaggatggaatttggagtccatccttcctcagtgataaggagtttttatagccacaggcagcatctattggtctgtcctctgcaaacttgcaactcctctgagagctagacttggaaatgaaacattattttgcaatgcgctgctatccttcatttttagctcctccaccgtagatgatagtttgtacttgttaaatgataaggatataaatttaggtcattttttatattttattgggtggaatttggtataatttttagacttcaggctttacaggctcctgagatggactgattgagcttgttctacttcttccccatcatgataggaagtgctgtaccacactaggcagtgtgtgtagtgaccacagactggctgagtgtctcccatcccatgctggcccatatctggtacccacctgatccacaaatgttccatcagatcctgttcaaacaacacatctccagttaagccaaatcttgccctttctccttacggtaaaatgtactaaatctgaaggttttgtctttttaatgttgctccatgatccagtgatctgtggccttggttatgctctgtgctagagtcctaacaagacaaatgctaaggtagaggtcattctgctcaaacaacctgaccccacctggatgtgggcttacatttgcaaagggcaccaaagttctaagagatgaggggaggagctgagccccttgtccttatctaggtttcccttgttctttcccatccctcagtctgcttcttttcccagtaccaacatgtttgtgtcctcagaattaaaggagtaaaaatgtgtaaacatctgactagcaacagccatgagattttgcctggcttgttgataagcagcattgagatctgccctcctaagaatgggccattaggtcttcaaagcttttacgatgtgaggtaaagaatgttcaccaggagtttcatgcacaaaagggtttctctttgtgggaactagaacattgttccagtgatgacggaaacagggctttccataccaaaacagggttttcctttgaatgactctcccacctttcccttgtctcttcctccccacctcaacaacacaggaaagaagctggaagcagggacaatgggaaggtccctttgttactcgagctattagaaacaaaaagaaaagtggccatctgaggaagccacagctggtgaaactgtagggtcacagagtgaattacacctctggcttaagtcagtgaaaagtcctagaagtttgtggtcctagaagtcctaaaagtttatgggactttgttttgagcaaggataagaaattgatttcaggctgggcgtggtggctcacgcctgtaaccctaatactttgggagacagaggcaggtggatcacttcaggtcaggagttccagagcagtctggccaacatggcgaaaccctgcctctcctaaaaatacaaaaattagccaggtgcggtggcacatgcctgtagtcccggctactcaggagactgagcaaggagaatcccttgaacccaggaggtggaggtctcagtgagctgatatcatatcactgcactctagcctgggcaacagagcaagactctgtctaaaaaaataaataaataaaaaagaaattgatttcattcttctgagaactgcaacaactaccttaaagtgattccatccaaaacccacatgttcagccatggacttgcttttatggagctgcgtgtgggtgacacacaaaatcaggagctctgagtcctaatttagacttttatttagatttcctcaaatttgggttccagttaagcgtgggtctcttctgtgccccgctcccctttgccatttgttttatctgttcttcagtctgttctgtcagtacccacaggcaggagagcagaaaggagaaatggcagccacagcagacaaatggcacattcgttccactcagctctcgcatgcccatcacagatacagctcattggtctcttttctatgagaggaagccagagctccagggaactactgccaactgatcagaactcatttaggacatggacctatttgttcctttatgttcctgggaagagcacaggatgaattctatgtactcatttacgtgttcagagagtaaagtgcctcataggatgcctccagcaaaagataaccaagaaggtctaatacctttgacaatctcagtttatcctatagtgtaattggatagcagttcccctagcaaaagttgctagtttggtcctattttctacatagccaaagtgattgattcattggttaatgtgaaagttactgagtactgccagcaggttctaggaaatatatttgtgtgatattcatggatggggaggatcaatccacttccaagtgatttggattaattactggtattttcacctgtgtgggtagcaaacctcagaaaatcaagtatagatgacggcataggacaggccaggccccaggcaaaatgttgaagctcctctggagttccctcccatctccctcttttgttttccatatacctggtttatccagggccctggagatgctccaagaccccctacccaggtcttcctcccttgtcccagctatatttctccatattaccactcttctcaccgaggatttgcttacttaacacataataaatactattaaaagagaaacttaggcacattaaaatgttagagttgattccagcaaacagtgattcacaggaggctccagatcacaagtggttcagggccccactgaggggtagggaagcaagacaaagaaaaacaaagcaaatatttgattggttcaagtggaaagtccctgattacaggttagtgggcagtttgtgattagttaagtttctctaagttgggttttggtttgctgatgtaggaacacagaatgctggggccgtttcaacctaatggtctcccaattaatttttttaacattactgatgactgttaggagtctaatgtgctactcctcccagggaaaatggcattcctaggattaaaggaactcagcacatggagtgtgcgtagaaatttagacactaactgcaggctggtgggagagagccctttagggcagaatgagaaggcgtccggccaagggcaggagttactgacgcatggcctcttggtttcagcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtggtgaggcccttgggctggcccctgtcctacaacacgtttccttggaagggtccgtagcagtcctggaggcccagcctgccctctgagggggtccactttgcctttgacctaaggttaaaaagttcacgtgaggctaaaatgtacaggggcaaaagtgggagcagtcctcaccccgagcgatgcaacagtgactcctcaccacgcctgcttgattcatctgccctggaaagtcattaaaaaaccagttcaactcatgggtccctttatttactcacaagagagagccagcagcccatttcactagttttcctttcctactctttgagaagaatcagaagggagggagcttgccactttactatctgtctaaagagatgtttccattaattaaaggtttttgttttgcttcaaaaaaacttgaattggagtatttccacaagtatctttaacatgctctaccaatgtttgcagaaagaagtgcagaaatgagactgtccacagagtcaggctcgctggccaggagaggactcccgaagctgacttctgatggcctgagaaacttcctagttcacaattcccagacccagacaaagagcactgtcttttctctaattgttttcaaatgggccatttccaccctctaatcagcctctggccctggagggtgcagttccccttgtcctccggagtctccctgtctctgtgctgtagagtcaagaagggacaaccacctgccctcactgggaaaagacagaaagtctgacttgttctcacgactcacacttattaggctccagaggtgtcagggcatctgcctttcatttcttaggttaaataagaaatcaattgctgccatttgtagtacccaattttctaaaatgatcacaatggataagtggcaagaaatccttatgactcatctgtgggcagagttgggctattttggtaatccttgagtaggcagatggaatttgaggccatcttcttgggtacatagatcactaggaagctataggtctagcaactgtggattagggctgggctgagaattgtttcatgttttttgtgactgtatagctagagactctcttgtttgcagagagacactctgaactccccctggccgtcaagggaaagactgccttcaccctcctgagctgaccttacactgagagacaatggggaccctcttttggccctcccctctacctcgagggcatctgggtgctgttgcattggataaaaggcactgctctttttctgtgccctctccgcctcactgcagagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggtaagcatcctcctctatagggtaaaggtaattgagttcttcagatccccagccctctccattcatctagtttaaatttcatttcttccaagctctttgtcagaaccagcatttgaagtttaaatctagaagttaaaaatccaccagcaaatcctactggctctacttgagaaacaaatccagaatctgatctcttgtcaccacctccaccacaaccttcccaatgccagtctcttccttccactaccacctcccatcagtccattctgcacactgtattcagggagatcctttcagaatcaaggtcatgtggtgtcagccctctctgtcaaatgcttgcactggcttttcctctctttcagagtaaaacccagtgtctcaaccctggcctccaagctgcttcattatccggcctccaactctcttcttcatcttacgattttccctactcctccatgttcctctgctccagccacgtcggcctccttactgactgtttaatacaccgagcgcatttcctcttcagggcctttccacctgctgttctcatgccagaagcacatttctctccccacaacctgcaacccgcccctcatatctgcaggcttgcttccttactttgttaaggtctctgttcaaatgtcccattatcacagggatctttccagactgaagagatctacataactatggctctgtaaacaacattcctccagggttcctgtccccttaccctactttattttggggaacattcttcaccatctgatacaatgatgtatcttatgcatgtatttactgactctctgcccttagtagaatatgagcccagagagcatgcatgtggtctattttgttaactgtgacagtcccagtgcccagaatagtgcctgacctttggtgggcactgaataaatatctaagtaatctgtagcatggaaaatcagcttctgaaaattggctgtttgcacggtcgtgtatttgcttggtagaaaatcaaattttccttcaaattagcattttctggtaactagagctgccccatcttcctctgagtggtctccaagtcagccaatagccttgtgctgtggcagccatgcctggctcttgatgctgtagccaaaagcaggcaggggatggtgaggctggtccagtccatggggagggacaaactcacagctctcagatcatctcagggcagcctttgttggcagaaataggtaggcagccaccctgaataggaggaaggcttctagactgggtcaggaggcctgggtttgcatcctagtggcaagcgtgcattcatttactagggctgccataacaaaataccactaactgggcagcttagacaacagccatttatatctcacagctctgaaggctggaagtccaaaatcaaggtgttggcagggccatgctccctctgaaacctgtaggtgcttgggcactccttgacttgtagatgcttcctgctgatccttcgtctgcacatggcattctgcctgtcttacatggccatcttataaggataccaactggattggattaggtgcctaccttgctcccatgtgacctcatctcaactaatcacatctgcaatgaccctgttcctaaacaaggccacattatgaggtacctggggttagcactctggtatcttttttcttgacagcacttctgacaccaaatgtgtgttttggttttttgttgttgttgttttggcaccaaccaattctcctatattaatgggttgtccaagaattcaattgaattctgacactatccagaattcacacagactccacgggttcagtcccacaaggcttccccgtcttcagatgccagctggaaatgtggtgcccaggctacccacacttttgccaaaatcctgtacttacaatcacagctttaaaatgaaggatgcagctcaggaactgccacatggaagagaagcacagtatggggtcgggggaagagtttctatgctctctctagacgcaccactctcccagcacctcaaagtgttcagcaacccaaaagctctccaaatcttgttgttcgagagtttttataaccctatctccagctccatactcccccattggaggttgagggttgggactgaaagttccattcttcacatgtgtggtgtttctggtgaccagtccccagaaactgcagctatcttggggctctaccctgagtcacatcattagcataaactcagatgtggtagaggaaggggcttattatgaataaaaaaagacactcctttctgccaggaaattccaagggttttaggagatctgtgccctgcacaggagctggggacaaagaccaagtatattttgtgttatgccacagaccccaacatgtctttttggagggagaccaaattcaacccatgacagtgactttgaacaagacatttgaacttagtctgttttttctatcctactagattgttggaaacagatataatagatgaaaattagttgattaaaattgaaatttgtgcataattcaaaagttttattttagccaagctaaagctttcatttattcaacagctatttactgagcagcacctgtgcatgaggctcagcagggccaggttctgggaacagagcggtggagataaagatccagacctgccccgaggaatagacagtccagtggcagcaaaggccatgaaacatacggcaactcttaaaaaaagccgagaccatgattttacaaaatcaacattttgtagggagcagaactttcaaagagaactggactagaaatttgggagtctttttcttggaaccctggtagatccagtagaatgagggatgggggtgtagggttaaaaacactgacattagaactggattacctgtgttggaattcctacatttctgtttcactatctgtgacggggggcagatggctgaatctcagtgtgcctctgtttcctttctcacaagaataatattactacctatctcctggggttgttttgaggtttagattatttaacacatggaaagcactcacagcaatgcctgccacagaaagaatatccagtacatcttagtgatgatcaccattattattatctgactcctggaaaaggacttgatttaattctctcatgaaacgttttcttggaaaactgatgtcaaccaagattattggtcttgctgttgcttataacaccccaaaaacatgactgtgtggataaaaatatgttggaaggggtagtctttctgggagcctgagaatagccatgtaataataactgcaaatatctatagttacaatttgaggttcaggtaaataaactctagatcttatagaactgcggtaaggtaggatagggagactccttcgactttctctgtttatttgtctctatttttaggagactatggaaccccacttccttggtactttcttctacaagagtcgtattggcttggcggtgaaggtgagtcctttaaaacacaaatcttaatgtttgaaatcaactccttgggctctgtgcaagatgtatatggatcacagaggtggccctctatgtaaacggtgtgattcctgatgagtcagctgcctcctggggctctgccccttgatgggcattgcagcgtctgggggaccacctttcacaagttgctgggccctgtgtgatcatgaatggctgatcatggatgaagccctgggtcctgtacaccttgtccagtagactaaattgccctatttaaaaaaggccaagccacttcagggttcaaagaacttttgcagcttttcagtataaagcagaaatccagggaatcatgaaggaacctttgcattcatctcccattgccttccttgtgcctttttattcttctctgccttttcaaaatataaattagtttattctcccaagatgaagactcctcctggggctgaggcagagctgttatcttcagggcaatacctcagattctcctggtgttgatctttcttaggggtggggaaaaaggctgaaagggcatttgcccacaacacatcttaggtaaaaggcacctttactactgaaccaaacaggaggcctagctagagaaagttctagaagcagggaaaagcacagactcttttgtgaggtctgagaaagcaaagaaattccagggtgaaagcgggggactcccctagagctgaagtactctcccatctgtttgttgctcacctacctattctttactttgtattattgggcctgggccaggacttatcctgcaagcactgagatggatgtttgttttctctgggggattagtctttttttttctttttttcttttgttttttgcttttgttttcactgggtcaaacaaacaacactttaacagctcaggattttttcattgtattgacttgtctacctgtaaacttgttaattttttactataataaaattatcatataataaatgaaaaatttcaacacagggcttgtgggcattttatttttctctacaatcccaacagatactctgcctcttaagaaaaaaagaaatcataaggaaaatatgctccttcaaaagtgaatcacaaatatgtttgccaacggaaggcaaatatttttcacctgtctcataggctggactgaaatggatttctaaaactctctaaaaccagaaaagagctgagtgtctccacccaacctccctcctttcacagattaaaaaataaaaaatggagcccaggagacatccagtatcttcccctattggtcacctgggacaaaatctggaacatgcacatgcattgcctggcaggaactcattccagtgattaaactcttcaggaggatgtttcctcttgctatttcattacctatttgtgcagtttgatagctagtaaagtgatcaaaggaactgtggggcatagattcaaaagtccttcaggaagcagaaatagaagaacagtactagaggcagcaggtccctgaccagcaggcccactacctgctgctccagcacacatcctgcacattttcagagggtgggggacagaggggccctgggtggctgttgcattgagaaatctcgccctgctcctgtatgtgcacttgaggccgagagcccttggatgcctggtgacagtggtttcctcctgcccctgccttcctctctggcagactgactggcccttctgctcctcttccccttccaggatgtcctgatatctttttaaaccaaatgccaagtttgccaaaaagtgtctgtttgtgtgtgtgtgtgtgtgtgtgttcaatgcgtgtgtttataccacacttcacaatttgtccaggcttgtattaataccatcaccaggctcaaccctggtgttaattccaagatacttaaatgcccatctaggtgaatttctcaggtaaaccatatattcaagctgtagtttaagctggctgcccgtcatagcactttgaatagactttgtttttgtttttgttttttgagacagagtctcactctgtcggccaggctggagtgcagtggcactatctcggctcactgcaacctccgcctcccgggttcaagcgattctcctgcctcagcctcctaagtagctgggattacaggtgagcgccaccccacccggctaatttttgtatttttagtagatacggggtttcaccatgttggtcagactggtctcgaactcctgacctcatgatacgcctacattggcctcccaaagtgctgggattacaggcgtgagccaccacatccggcccctgaatagacttttactcaaggttcaccatgactttcacatgttttgtattggagtaaaatgtgccagtggtgggctaaagaaaattaactcatttcaaattcaaacctggttttcttaatttttttaaaatcacagtttctgaaactgtgggctcctcatggcacattgagaggaggaggtgaaactctccaagtctgaagctcctgttataaatcttcctctggcaaagattgtgtgatcaggcttgagtacctcacagtcctagagcaggtcaaaggctggctaggaaactcatttgctccctgtacctctcccctcctttcctgcctttgctcgttctcagctcccggtggtagagtaacactggcttctgattggtgcagggtgttcaaccagagaagaaagagccctggaaaagaccgagcccctaacagaggaaacggaggatccagagcacccagaaggaatacacggtaaaaccccgataaagaatacacagcagaggcgaggaaaaggctctaagcactgcagagggccagagcaaaacatctcatggcaagggtggaaagaagcctaggaaactgactctctctgtggacaagtgttaaaccagatcccttctcagaggtccatctgcatgtgtgtggaatgaatggttcagcccagacattagcgcatatttcctggagaaagcaaataccaactatgtagtgtgcctgtgcccttgttaggcaaatcccaagtgagttgcacaaatgtgctgacttccgaggatttagcaagaacaataactttggtcactgggacttaaagcggatatgagctataaggaaagacaaaaataaatgcttctgtgtccagggggaaagagactccaggggagctgactacacttcacttacggcttacaaatctagaaggccattcattgaaaccatcagaagcctttcctgacagtggaagttacctaataatccctaaactgacgacccagatttacaagttttgttttcctggcttttgctgccctcatcttctctcttaaactagttctgtatttctcccaaggcttttcattccctaagcatacgcatttctctgtggccaaaatgctctgggtttagacaggcagcacagcccctgggctctgcctgacagggcaggagagggtctggcctttatccctccagcccaccccaggggccatttcataaaactaaagccagagacctgcagcccctcccagagttagactgcagtacaccatgcctctggcaagatcctcctcccacagtggaaagtctaagccaaatcaggaggctggggactggttccacctcagttgcaggcaaggccaggaggcacggatagaagaaacagtggactttttccccctagggaaagaaatgcttagagctacagtattaagatgacaaattaagctgtgccatatagggtgaaatgaagcagggatagatgggaggtcagggagaagtgagagcactcggtgagggtctgcactggagggggcatgggaggaagaaggaggggagtggggtttgagggatggtgatgaggaagcgtggactgccctacccacctattggaaaacctgggagttctgaggagcaagaagccttagtcaaagtcaactcaaagattcaagccaaggtgactaagagaatggcggtccagaaaaggtcatgggagaatctgaaggcagatgttgttttgggaagatgaagaacctaagccgcttccagaaattcatgaggaaatgccccgtggactgttggcaatgagggcctaggaccaaggttgagcttggggccaactctccctatagacagtgagtgcattctgacaagcatgggctctgggttcaaatcccaactctgccactcatgcctatgtgtccttaataggacgcttgatgtctctgtgtctaaggtttcctggactatggaaatgagcctaataaatgtctaccccttaggaccattgtaagagtacattgaggtaatttgtgtaaagcagtcgaagcagtgcctggcatataggaggtgctgtataaacgtttgatgctagtattactattattattctggagtcttccttgcaacggtgatagccgaagccacaggggcaggtgacgttataggcagaatacaagggcctggagacagagccctggggccatgtaattaggcattatgtttacatcatgttcattttttttcctccaagactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtgagtcttccagcagagaagctggctgccatgctagcctgtcatttcctggcttagtctttccctatcagcggctgtctactctttcccacaaattttagtgacaaatatttgcggccccaaaaatgtgtaaaagctttctgcagtattcaaagatcactaatatgtattctcttgatggggaggtagaatacgtttattgccccttttgtgtgccggggaagtggacattcattcagagagttgaagtgactttcctgaagccaccaagttgtcatggctcagcgggggcaaaagccaggcaccacagttgcctcttgtttctcacaccttgagtctttccccccatctcaacagtccatggtggtgatcaagtcatggccactgtcatcatgtgcatggaagctatagagtcctcctatttcctttctcttttcttttctttttttttttttttttttttgagatagtaaccattacccatgctggagggcagtggtgcgatcttggctcactgcaacctccgcctcccaggatcaagcgattctcccacctcagcctcccaagtaggtgggactacaggtgcataccaccatgcccagctaatttttgtattttttttttttttttttttttttagtacagacagggtttcaccatgttggccaggctggtctcgaactcctgacctcaggtgatctgcccgcctcagcttcccaaagtgctgggattacaggcgtgagcgaccgcaccaggccgagtcctgctattttcaaggaacattccttttcctaccaatcattaggcaggcttcaacatcagctgatgagggttagtggtcgttctggagaaagtgaaaaaagaatcagtctctagaggggcttgtggagtaaccgcctggtaacagaaggtcagggcagggaaggcaaaggggctctgcgcggatctctcagctccgcaggcgccccactctcctccaagggacccgagcgccatctgctgagaggagaacacggcccgccatggtttcccaaggagcagcagacacggacctcgcagggggcagcgaacccacgtgacacagtcttcaagtcctttggagagccccaggaaggaacaacagcgtgtacaccctgtgatggaatgttctctagggcggttcagtgtgaatggaatgtggggccggtgccattctaattggttctgtttccctctagtggttgatcgcggagatttcggcttctccatcaggacaagttcagatagcctgagatggtatcagaactcagggacagagctgggtgtggcggccctgcatccatctgctttctctccatgctaactgatatggtcagagagctggaagcaaattccaggaccccagggctccgcaaaggcaaacacattacttcatcggctgctgacatgcaacttcccccaggggttaaaacaatgtttaatactaacagtaataatatttttgagttttactttatgctggcgctgttctaatgttgtaagtgtattaactcatttaagccttacaacaacctaaggacatgggagtcatagttcccatttaaaaaaaaaaaaaaaaaaagcccaccattgctctgaggctttttatgttttggatccaaagctaatattggtggtggtaattcccatgcctggcttcgatcaattaatcagcaaatgcctaggactgcttagggttctggccttcatcaagaccttacccgggctttatgatgatgacacctggcttttcaatagccatgactgctcacccaggaggcaacgcctcgagtcatgcaccgaacaccttttattgatcctctccaacaccaggctccgtgatggctgagctggggacacctgtgactgcacgtgaacattttgaggctgggaatcccaaaggccctcggcgttggcctgggagcaccatgaaacaagtagaagcagagaaggatggcagaggtggccctctgcattagggcctggatgtatacactggtgctaagggggccccacagctaataggggtttgagtttgactgacagccccaggcaggaatctgtgagagttctcactgaacctggtgtgggggtggccctcctaaggcatgttgctaaaggccatctcttctgccactgacgcctgtgttctgcaggtccatcctgacgggtctgttgccaccaacctctgggactgtgctcgttgggggaagggacattgaaaccagcctggatgcagtccggcagagccttggcatgtgtccacagcacaacatcctgttccaccagtaagcgacacaggaactgagaccgccccatcccctctcctcacctctgcccccagcacacttctctagagcccagctcaggggtgccaggcctgggcacaggcagagatacagactcttatttggtttcccctatgtttaaagtcctttgtcctacttgcagtgagaattgtccctgagaatatgggactctgcctctgctgctcagagctgagggctcctccctcagaagggtgaggctgccttcgctctgacagagcagctgatcgatccccgagccccttgtgcagccctgaagtacttcctctctgggaccaaagacaggagaaccattgttcctttttcctgttgaagccacggcctgaaaggcaaacttttcagggggcttttcagttactttttttccccaataagatatcttttatttcttatctaagaagctacgcatagtcattgtgaaagaaaaaaaaggaagggaggaaggaagggaggaaggaaggaaggaaggaaggaaggaaagaagggagggagggaggggagaaggaagcgagggagggagggaggggagaaggaagggaacaggagggaggaaaagggaaggggaaggaggaaggaaagggaaggagggaggaagtaaatataggtaaacaaaaaattgaaaataaaagtcacctgtaatttcactactcagagataaccgctgagttataacattggtatataattttttagaactttctcctatacatgtatagatagataaacacatatacttcaaaatgataaagaatagtaaaactatgcatacaattttataacctgacttttttttcaaaaaaaaggattgcttttttaaacataagatatcaggaacatctttcatgtcattacatattcttctataaaataatatttaatgtttacagattattccattgtatgcatgaactatgtaagccatcctcttattagatatttaagcagggtctgctatttttgtattgtatcataaacaccaccacagtgagcatcttgattgccaaatcaagaatacttgtcctcaattatttctgtaagatcagctgctggaagtggaagtgctaagccactgcttttctcgttgtcccatcctcctagcctcacggtggctgagcacatgctgttctatgcccagctgaaaggaaagtcccaggaggaggcccagctggagatggaagccatgttggaggacacaggcctccaccacaagcggaatgaagaggctcaggacctatcaggtgctcagagctggatggagacagggccacagatggcaaatccatggctccccagtgcacccaggaggcaggggaggcttggagcaggagagcttctaagggtgggaacacctctgtgaagttacaccaaaaatctaagagcagcccccagatcattttccctgcagagcactgtctcacagcagcctgggttttatttgtcctgagattgatgtgcttgaacagtcttcaaagggtctgatccgaggaggtgagggttgccctttctgcatttacaaagcctgaacagtattagggctttgaacgctataaacatctaagaggcagcaccaaaccactgctgggttaaggtacccccacaatgccacttgccctgggcctttctcttcctcaccctccacagccccttaactctcccgtccttcttgtgcctccaggtggcatgcagagaaagctgtcggttgccattgcctttgtgggagatgccaaggtggtgattctggacgaacccacctctggggtggacccttactcgagacgctcaatctgggatctgctcctgaagtatcgctcaggtaacagctgctgctcagtctcctgggctgggctctcactgcagccctagctgtggtccccactctctcacctgccattttgtagctgagtacaggaaccacaatgactacactcagaagggggtttatcagtgacttggtgaatctaagttccagctaaagcctcctgaggtttttacaaatataaacagagaatcactgatgatgcaacctacttcccaaaatattttagaaaattctcttgacctgcagcccttctgtctggaataatggatgctactctaggtgaatgtcttctctgaccatggggacccaggtcacctgcaaacatacctagaagctccatagctgtcagatgaccactcaggaccagtgtgagggtgacctgctgggcattcagtgctccagagggtggccacagatggaagtggctcctctgtcatggcacctctcagacaaggggctcagatcagaagagacagcaagcagagctgagtgcccatagaggtaacagcacggttcaaccccgtggtcaagccagagctttcccccttgctctactcacacagcgttgccccgtgcctttctctgagggtttgtcatcctgaaatcctcattgctattttctttctttcttttcttttttttttttttttttttttgagacagaatctcgctctgtcgcgcaggctggagtgcagtggcgcaatctccactcactgcaagctccgcctcctgggttcgagccattctcctgcctcagcctcctgagtagctgggactacaggtgcccgccaccacgcctagctaattgtttttgtatttttagtagagacggggtttcaccgtgttagccaggatggtctcgatctcccgacctcaggtgatcctcccgccttgtcctcccaaagtgctgggattacaggcatgagccaccgtgcccggcctgctgttttctgttaatgacatctccagttagtgagagtatgcacgtgtgtgttctttatgaagagtataaatccagagcttaatgatccagaaaatgtacatatgaaactccctagatgctgaccataatacatgagcccctaatatagagatttatttgaatcagatcctatgctggatacagagacactgtgtgtggcaatgctttacagtatgtaggaagctatgaaatgttagttattattgtcctaatatgctggaatttgctgctgaattagttcccttgggtttttttttttagttaactcctgatttttgcaactatatagccaggaaattgctgtacaccctttaccaacaatgcccaacccagggcaggcctggtgattgccctggcccctaccttgcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggtaggtgtctgcccagggaaggaccctggcctgggtgagaaggagcacacagcacggggctgccactccagacatggctactcacacaggctctcgccaccagaatcagtgtctttgttctgggaccatttgcagaagatttcgatgaacacattctgaagcctcctcctacagagatgctttagccaaaatgaaacaactagctttaaatggtctgcaagtattacatgccagattacacaccagtttggtgcggtttggtgcaacatagaagtgagtgtcttattctgtaaggttaggctgttttaagagcaattggttgagcttcatttcaacattaatattccctaattaaacctgaatttcagtggtaagtgaaaactaagaagaggcctccttgggtgctataacataaaaatgatgaaggcaaaaagtaccaaccagcagagaccacttcagcacatcaggagacccagttttatgtctgtgctgcgaagtgaacaaactgtgtcatcctaggcaaattatttaattcctccttttttttagtatttttttcttcttcacatggaacatgaagctaatgacctctgcttctatttcttagggatgtgaagataagtgagataaagtattataaatgtgctctgggcttcttaagaacaggcattgctcacattcaaatggtcatgattatgatatggcagcattatttatgcctctggtttaagtgtctggctgccgctggggtttcctatgtccatccacggggagggaggcacagaatgtctcccacaggcagaacctacagctgccacataattgatgacaagccaaagggacccttggaggttctgctcctctctgtgtgtgactcacacactctctaggataaaatcaagcgactacaccctcaaaatgctcagatgaattaacagattaaacagtgaagaaaaaaatgtgttgactacacttggcagtgagaaataaataaagcgggcggtgacagcagctggcatcagggagaggctgtcatggaagggatgtgcatcttgtcagtcatcccatccatctgttgcaggggacctgcagctgctcgtctaagggtttctccaccacgtgtccagcccacgtcgatgacctaactccagaacaagtcctggatggtaaggactggacgggccatacttgggttccgtctggcagccatctcccagtattgctgggtgtgtcctgttgtgatgcattttaatgggagcaaagagaacactgggtacttctgcaggtcacacagttgttcttttgctttgagcttctttctcctcttccttcttccttcattcccaaagggattttaaaagtcatgcacctaaaggccctctccctttaatgaggaatacactctgtgctcttacccttagtaagccatcattcctggggtccccctgccctggctccaggccacattccttagtgtctggggagagcttcttctacatgtgtgccgtggcgccctctagtggaagcatggtgatgcacggctcttccagtgaattcgtggagtcagagattgcacatgtggatggcaagtctggaaatagcatacacccctgttatactcctgattctcccctcagcttcccaatttcccagtgattctccctttaattaggatgcactgaagctctcaggggtgcccccatctccaaggagctgcagtggagaggctatcccctctctatgtgagagaatgtgtgagaagcgtattcccacacaggagcaaaactaaacttacgtactgatgcaggttaatgaatggggaaagtatctgcttatcaaagaaaaggcatatttttctatttagcacaaactttttcaaatgttaagaatttactaactgaaatctggtgaagcaagagaaccgggcaatatttgcgttgtctgatcattacaactggagggaacatgctcagagaggcatcatcactgttcatgcacctgccctctctttacactgagagaccctgtgatgaacagaaaacatctttttaggatgacatctctgggtctttctcctagcctgccttgctgtgggtacctatctccctgctctctgaaccttggtcaagaagtttatatttgttttaaattgatactaatatgttaagttactgtgatttgccaaaatcagattggaaacagggcctgcatggctgaatgattcttttttttaaattactttatttctaaataaaggttttctttgtatagaatcgggatgctgtgaatggtgggaaatgcactaaatagttatgccccaaataagaaagggaaaatcatttgaatccccagttagctccttgaaagtcttttcacttaaacacacccacataccacacacacactcacagacctccctcccagatgcccaaagccctgctgacctacagagctacttctggaaaggctgacacatgcctaagacacaattcctgggaatccagcagctttgggttcaatttccttcctaaaagaacaatgaatatgacccctggagagctattagggcagagctgcttccttaacgtaaaggactctccagcctccgtatgaagtcatctcagagctaaagacaatcaagtccaacttgcagatttgacataaagcaagacttccaatccggctaggcagaaggattttggttgaaaaccatgaaatcccttcatatggatcattttttaaacaacaaaaaaagaaaagaacctactgggtgtccacaactctgagagctgctttctgaagagtcatgttttgagtcctggaatccctctccctttgacctgcctctcaagacaatgtgcgagagaactctctcttcaagtgcatgcaagtgaggttttcacagttagatttttaattttaaagtaatacacatttgtacataaaattcaattctgactgtatacatgtgtcagataaacagttgatacctgacacttgttcacagtctatgatacgcaccgcatatcctaccctctcccccagcctctctccatggcttctcaaccccccctctgcatttcctgtgacctgaggattcagttttgtttgtggaggcaggtgcaatcccaagagaaactgtgcaatcttctgagaagttagagtaggcatgtgtgtgtgatttagggaaggtacttctcactcagcttggtcaccggttccaggtttgtgtcttgggcaagtcccccatagctggtgacagaccagaaaaatgaaaacaactttgacttagccctcaagttttcagtgaatgagaatgaaaaacaaccatgagtaagagatttcttaccgagatgatgtaaaggataataatagcagccagcactcacctatgtgccaggtatttctctaactgctttgtgtagtttgactcatccagtcctcaaaaacaacaatgaagtggataccagtattttccccttttcacagatgaggaaagtctaatgtgacccacccaacataacatagtttgaggggacagagcatttcgttgaacagaggaggaactggcacaggaaagttgcatgacccccccaccaacctccgcccccaggttgcacagctagctagtcgggaggactttgcttccgtttccctctgcctctcaatgatgatctcagggccaactaagctaaaagcagacttgatggagcatcagtcctctgaaagagtcactgccgagatacaaaatacctcttcttcaaaggggaagtggagagaagtaggaaatctgggtaacctcacagtcttccagtttctggaaaacagagctggcatcagtcttttttcttgtcctaggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagaggtaaagtagagattccagctggtttctgtcaagtgccagaagtggcggttctttgaaaaagtctaacattagagcaaagttttgtaaaagcaaaaagccatcgttccccacccaagcatagcaactatctttatttttggcatagttcccccatctctgcatgcatacaaattttatgtacttgtggttactgtgtgcttacgtttttgtatttatagaagatgatgttctcagatagagtcgtaatggattttcttcccattatgaagcaatacccaacaaaacagagcttgggttagatttttctgagaataagaatgactaaacaaaattctctctttttttcttcttgacagatttttctgaaggtcacggaggattctgattcaggacctctgtttgcgggtatggtgctggagccagtggcttgttcccttccttgcctccctcccaagttccatctcgaaagtctaaggggctgggcacagtggctcatgcctgtaatcccagcaatttgggaggccaaggcagatggaccacctgagttcgagaccagcctggccaacatggtgaaaccccatctgtactaaaaatacaaaaattagctaggtgtggtggcgcgcacctgtaattccagctactcgggaggctgaggcaggagaatcacttgaacctgggaggcagaggttgcagtgagcagagattgtgccactgcactgcagcctgagcgacaagagcaaaatccatctcaaaaaaaaaaaaaagtctaaggaaaaagtcatgaaacaacaaagcaggcaaatactcctccatagtatctgactccccagtagtaggcattttgcatcctagatggctttgagtgacaaaggaataacagactgagttaggtctagatggggacactttggatgaatgaggattcttacggaggtcaggttggtagcttcatccctcagctcctcatgctgtatccccagtctctcggcctgccatgtcatcatcctcatctcctcctgtcatctccaccaggcctctgatccatctctgtctgcatgagtgacagctggcagagtccttaatgtttatcaaatacaactcagacgtcagtctcctggcccctttgagatcaacataaaatcattttgaacccttatttagtggtctatgggctttgaaaacatggggaccaaaattcctgtggattctagaagtctctcttctacatgtgtcagcctgggcaccaactagctccttccatgaacttttatcaaacccacagccacacaaagcatgtgtgagtgtagcagagtttacagcagagggtggagggtggggagatagatgtgtggaagggttacctgccacacaaacagaaaccacttctgatagaacacgaggtgtccacccacactgtaaaatcctctcctggtacaggcaaagctttgcagcgattctcctttgctgcccctgggctcctaacacctcctaaaccaccagttacctccttctttccagtgtggcatatttcagtgttttcctgttggagtgtttcctttctatgtggattctggaatcagctcttaagataacttggttttcatctttcttcataatgatcccaaacatctatctactatgcctagaactaccaatggacacatataccagcccagatatgcttcagcccatcccagtacatcgcatggtgaccaaaagatgtagtcgtcctggcacagtgggtgtggggcaggaagcagtcctctccaggggacagcagcaattcaccacagaacccaagtttctttcaagctctgctgacacagaaattgaataatctcagctcacccaatgtcaaagactcatattaaccaagaccagaatgaaaatatgctaatttatatcagaagctttgctggattcaagagttagggccttttacctgtgcagaatattccttcttgataaataggccctctcaggagaataaattacacatcagaggactgtttagtcagcataggcatagaacaggatgttccaaagatacagtcaaggggagtgggtaagagtgtagcctctggagtgaggccgaccaaatatcaaacctgagcttcataatttgcaaactaactggctttgggtaagtacatagcctctttgtacctgtttccccatctgcaaaatggagataataatagcatctacctgtagcattgttgagagaattaagtgagttaatgcttgccgacttataacacagtatacgatcactgattaagacttagcaactctaaactaaatgtttacaaaccatctcttacctcaaagcacttaacatccattgtcttatttgattatcactgtaatcttatgaagcaggcagggcaggggtctgccccatctggggggaactgagctcacagaggttggagggtttgcctaaagtcacccaggccactgggtctcactctctggtcttagctctgtaatctaggatgctcaatgccacactctcagccacttttcagatggctaagtacatttgttttgagttagctcagtctcagaggatgacattttctgatcttgtctccagtgtttaaatgaacctgtagctgtgcattggggtcacacaatgcgtggcatggagagggtctgtggctgactgccacggttactacgtgaaaccatcattacagcagttactactgttactgcctgagaacatcattacaagactgaacgaagggatcaacatggaaatgataacaaaaaaaccaaagtaactgttttaaggaaaggctagcatcgggaagaagaagagagaagaagagaagaagaaaagggctccctgcttctaatgagtaaaggcagctccctaagcttctgcagcccttcattatttattgggtaacaggaggaaggagcaggaggtaatgattgggtcagctgcttaaatgatcacgggttcatgttgttactgacagatttcaattatgcctaatcataagaaacatttgtgcagcctccaacaagggtcaatgccacttctgaaggggtgactcatagtcagtaactagaaagcagcagatagctagggacaaactggcgattctgaataggcctggaacccttagctctggccaggtcagtgggctccagtcaggatggagccttcagggagagatcaaagctcagaggtttgagatgatatcagccagcaaagaggaggggcagtagggatcctcccagagggagggccagccatagaagacatcaaatctgagcccggatcaggagaaggagcctgcagaactggggctctggcaccgagaacctgcagaacttcgcccctctgagtgcaggtgccagggctggggctgccacccagccttcgcatcccaggcctggcacgtcataggtaaatgtagttgaaaggatgactgagctgatccaattccctttacaactgtccttgtcctgggggacttgaggagggttaagaaagcagctggggaccaaccaacagtcctctaggctctccatgtccagcaatagttgttcagcaaatgagcattaatcagtgactataaactgtagcttcaacataaccgacaacttgcaatggtttctagagcatgctcccatgtgttatctcatttaaatttccaaaccaatcctgtgaaatgttctttttttttttcttttttttttttttgagatagagttttgctctgtcacccaggctggaatacagcggctcgatcatagctcactgcagccttgacctcctgggcccaaggggtcctcccacctcagcctcccaagtagctgggactacaggcacacgccaccgtgcctggctaatttcttttctagttgtttgtagagacagggtctccctatgttgtacaggctgatctgaaactcctggggtcaatcaatcctcctggcttggcctcccaaagtgctgggattacaggcatgagccaccatgccttcattttacagataagaagtctgagaaaactcagatttaggcagattgagtcacttccccaaatttatgtatcttgtaagaatccatattcaaacctcagtcccctaactcttagttcattactttttctaccacttctcagtatcctctaagaattcagaaagaaccacatcgactctgatttttcatttgtttaagtacacaggtaataggtgaatgtattttgttgtttaaaaattcatataatacacaaaaggctaaagtctcgcttcccacttcctctcccctttctacccaactctgcctccccagggagagcttctgctgacagtcggtggacattctttcagagttttacaattatgtgtgtgtgtgtacataagatgtcagtttttctttgtgtaggatacatgaacatgaattttaaacataaatgtgagtgtattacacatattgaccagcaccttagtttttttgtttgtttgtttggttttctttgtgctgtttgagaaggagtcttgctctgtcacccaggctggagtgcagtcttgcaatctcggcttacgcaacctccacctcctgggttcaagtgattctcctgcctcagcctcccgagtagttgggattacaggtgcctgccaccatgcctggctaatttttgtatttttgtagagagggggtttcactatgtaggtcaagctggtctcaaactgctgacctcaaatgatccatccacctcagcctcccaaagtgctgagatgacaggcgtgagcctccgtgcccagccagttttgtttttttattaaccaagttacgtattttaaacttctccatgtcaatgcttttagagctattttgttctctttaatgttaatagagaattttaaggcaatttcaggtgaatctatacaatttctctgtataagtaatttacactagaaatagatttttataaagatgattaagctaccagcctggtatttcattgctgacttaaatgaagaggaaaatcaatgctgtaagggaaaaaaaaaatggcattagagatccagaccttataggcattttccaaattattaattcaatctctcaaaacaggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcaggtactattgtcggtcggtgtttagctgagctcagtggctcctctcccagccttcccctcctctcctgagtgttccttcaggcatgggttataactcagcaaggagcaccctctttagattctgctggttttgtttcctgctttccaaacccttatcttgattcttggtaacatgaatcttctttgtaagttggacctcccctagcaaagaaaatagaataatagtgaaaatgttaatattgtttttatttttacagtgagggataaagtcatgttttcattcatttttgcagtgaccctacatatcaaaatcattgccctcttttttcttttaatgttgtttaatttagaaaaagaagctctggtttaaagaacagtgagtcacgtgacttgctctttgaaatgccctttgaagtctggctgaacactgggctgcattcagattcttcagtggccaccagaacattctgttttcttctgcacatcttacctttgcacaccctgcttattatgttcccccagaagcccaaccctctccaccaggggctgattaggaggctgcaggataaatgtttaaaagaatgaagatgtgtgtgcacgcgcacgtgtgacatctccatgccacagtcatgtttattccacgtctattctcccacagatcgtgctcccggctacctttgtgtttttggctctgatgctttctattgttatccctccttttggcgaataccccgctttgacccttcacccctggatatatgggcagcagtacaccttcttcaggtgcgcggactcggggtcaccattctcctctgtgggtttggggcacctgggtcacatgctgcttagaagggccctgaccttcccacttcactgggaccttcaccaatgagagaggggaggggtctttgggctgcctgcagaaaggaacttaatgtatctgccactgcttggaaaggcgatcctagtggacaggcaggactgcttgggaaggccgaatggggaaaggaatgcaaagcttaggtgaatgggttgaagcgccatctttttgaggcataggtgacatgccatcagaccactgcgagtgttcaggcagcctaccgcactcccaggagagctagcgccatcccaaggcagcattcggtgcctccaatacatacctggcacacagcagctatccagtaaaggctctgagttgcatgatgttggcacgcgcctgctctgtcccagtcacatgtctcactctgtctagcatggatgaaccaggcagtgagcagttcacggtacttgcagacgtcctcctgaataagccaggctttggcaaccgctgcctgaaggaagggtggcttccgtaagtgcctacgcgcccctgtcctaagaagactagctcccctgggaggacccaacggtgggttcaagatggcaggcgttggggaggccccactcaatcctgctctgctggtcacttccatgtctctgaccagcactcccccaacctctccttccacacttgtgtgcagggacattcactacctcctaggaagcccccacaccactggacagctctatatttctcagcatagaagttctatgttgagttgacagatgattccccataacttatttgaaaggcctctgagcagggagggagggaaatagggttatgctattgtgtgattgggccttgaatggcgtgagtgacacagtggccagtactttgtgatagttgtgagtctggagaagggagttagcgaaggccattgacatccaccaggaatcctaaaagttcaatataattttaacttttctccctcagtctttttcaaagctgtcaataaggaccaaaacagactaatttcaaattcctcttctggttgctgtgtctctcaacagctagagctgctaggaataaaaagggagacaaaacgatccacaagctagagatggttattccccagccccacacctagtcagtcacaaaaccctagttttgatattgcttgagcagaaaccagcctccaagagaataagaagaaagggcctgggtctaaagaggaggaggaaagggttgggcacaatttcttatgcctagggatttgtcagcaactttgaggctgattatggaatattttcttgtcttccatgagggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccaggtacctgacctccaaacaacggggccccaggtctgcctgccacagagggactaggggagtccctggtatctcctgagtctctcacaaactaacatttcaaactggcagttgagtaggggactaaaccaaactccctgcaccctctgggaggggctccccacagggcgctgtggctgccaactggaggaagccactcaccaaaagcttcattttccaccagatacttcctatttgatctagtagaaaaaatgtgtttaagcactaaaaaaaattaagtcatatgtgctcattatagaaaaattagaaaacacaggtaagtcagaaggaaaaaaaatcatcgcttggatataaacacagataatgtttggtttgcagccacccaaacagattatattccaaatattgtcttaaaatctgatttactgcataatttactaggaacatgcatccatgtcaataaatagacatctgcatcacttttaatatctgtatattatcccattgtttgaatttcttttttttttttttttttttttttgagacagagtctctctctgtcacccaggttggagtgcagcggtgtgatctcggctcactgcaacctctgcctcccaggttcaattcttgtgcctcagcccccccgagtagtggggattacaggcatgcaccatcatgcccgcctaatttttttggtagttttagtacagatggggttttaccatgttggccaggctggtgttgaactcctggcctcaagtgatctacccacttctgcctaccagagtgctaggattacaagcgtcagccactgctcctggcctaaagttactttaaattaactgatctcccattattcgccacttaggttttttagttttcaccattataagcaatgctatgatgtacattcaaatggaaatgtgtttacacacttattaacagtcttaattaagaagctctccatgtgctgtgtctctaacatctgcaggtatgtacacaaatacatgcacagccagcatccatcttttgcagggacattaatgatcttggctctgagcagcaccctgtcctgggagttctaaagtccagaacagattacagtgagcatctcctgggggatttagagacatcaaagaaggctgtgtccgtggttgataatgggcctcccagctgacttgccagggctgggccttagacagccctgtccaatgatttgtcaatgaataaactgttcccaaacaggctatgcagttcagtgggaaagcacaggtatgggacacggagagccccaggtggactacttgacctctctgagccttaattttatcacctgtgaattgggaataactgcttatttcataatattattatgaggatttaatgaaatcatgtgggcaaggaattatttagaattagattcaactcaagtgatgacaaccccaaactaacagcagataaaacaagacacaacttgtttctcactcatctaaaagtctacgtgggtggtgcacgatgttctattctctttctcctccacactaaacaggcctcagcctcatcagccaataaggcaggagctgccttccaggcagcggaatggaagaaggatgaagcaaaacagagggcagagtgtgcacatgtgctatgtttagggaaggttttctgaagttcccacatagtacttccacttacaaacccaacaaaaaaggctatggctaaggcagcagggaggagcaaataatgggagcaactagattttgccacagcacctatcacagtctggtttataaatggttctaggccaagaacacccgatccctgctcttttttatattctaaagcatgtatctttatatttctcaagcaatattttctctctttgaatcacagctcatctgctgcatcatagggatcccaaaagaaggacccaaggaacttgtctcagtcctctgtgccccaagaggaagctttgcttgtttgctttgctgtcaatgctgagggctcctgtggctgcctccactcaaaaccctccagcatcaggacgtcaaggctgtgatactgtaccctgagctcttggccagggcgagggaggggaggccaagcctacctacatggtgtttcatttcctaaacgaacccttacttccacgcggtctgtccagcttagaaacttattttcagtagtgttggtccttggtccctggacaaaatgtaacagccaaagtcctagaaaaaggcaagccagttcctgccattttctttcacttctgcatttcctcactattatacgtgccttccattggagcaaaactgaatgccacgcatatgcacaggagctgtgcgcgctctgtctctctcactcactctttttctctctctctctttctctctcaatctctctgtctctatctatctcttactctttatctctcactctctcactctttctcactctttctctcaatctctttctcattctctctctatctttctctctctctctctttctcacacacacacactcacaaacccacactcttattcacatctgctcaccctagccactcaaacacaatccctcattcagcctggaataagtccagagggcgtgggcctgattcagagacaatcagttgttctcatctgggaaatggggcaatgtggtcatctctagggaccctccctgctctaacattctttgaatgtggtgggtcctgaggtggaagcactctgtccctgacttctagtatatgtggagatagggttacacaaatattttattgggcagaacttttataaaacaatttatcataagctatcgcagccagcagcaatttttccaacctggattccaccaggggagcttggccggtgtctgagtgccactttcagcttgagaagcaggtgactcagtgaaaagagcaaggaggagacagaggcagattcagttcctaggccctgggccacccacctgcaagtttgcagcccagtcagtgcaagtcagctaactgttctgaacctcagtttctctgtctgtaaattaagctaaaaattcttctttcaaagagtgtcaggatgaagtgagatcgtgtatgtagggcatttaacatagtgcccgacacacagggagcattcggtaggtgccagctctcctcctggcaggagagagagaaacaaggtgaaaagagtgaattaaagaagaggaaagtcaaatgggaaaacagggggaggagatagaaagtgtatgaaaaggaaagaatggtgcgcaataacggcggtgtaatgccaccaaaatcccctcaactacttctgggcagcacccttgacagagtgaatgcttttatgagaatgtaagcggaatgtgttcccagatttgcagtaatattgccacctggtggacaaacccatgcacctttgaattttccaaaatatttcgatgaactagcttccagtcctagatgtattttgaaagtgatttgtaaattgtaaggaactattcaaattctttcattaatgtcacaaatcaactgtgtcatctgtatgccacccactattctgggtgctggggacacaacagctcacaaatcaggcaaagtccctgctctcaccaaaatgatatcctacgggggattacagatacaaatacgtaaacagatccatcgggaggaaactctcagatggaaatgagagctatgaagataacacaacagtacatgacaatacagagtgactggaaccaggaacatttctccgaggaataaaatttgaagcgagccatgagagggtctacaggtagagttcccaggcagagtgaacagccaagcacaaagctgcaccaggagagagaggtgctcgccgagagacagggaggggagtgtggcaggtgagctcagagaggggcagggccacacacatcggccacatgggccttggtagtgagtcgagatttgatcccagggtttattggagtggataagtaagcaaggtgactgaggtgctcgggtttacatttttatagttcaagctggctgctgggtggaaaacggaagttggcagaccaaggacagaatcaggcagacccatgtggaagtttctctagtggtctaggtggtggcttgggtagcgtggcagtattggagctggagaaacgcagatggattggagatttgttttggagtgacgccattctgtcttgtcaatggattggcgaaaaaagaggcatcaaagatgagttacacatcattgaagtgagaactagggagatgccagtactttatttagtattttctcagcagctcaatccataaataatttttggaagacaacaagcagtttcacaaactacttataagtcctcaagttccaaggtaattaacgtgggtgtctcattgcctcagagaacacagcgcagcacggaaattctacaagacctgacggacaggaacatctccgacttcttggtaaaaacgtatcctgctcttataagaagcaggtaagaagaaatccttttatgctttttatcctggctccctgtagaagatattaactagggacagaagataattttctctctcaatttatgtatgatcagggcagtagatttttttcttttttatctgatttgagggccccattcaacataaaaagcaattgaggcacatacaagtaaaatgtaacttaagattaattctttttttgttgtttgtttgtttgtttttacatttagggcaagcagtcttaaattttaacccacgtattattaaaagttatatcagaagaccatagaagttattcaaaaatgcagccacatattttaactagttaaaagagagagtaaaaatttggagggaggtggaggagtataggggaaaaggtagaagaaaaagagaaaataagtaagtggcaaaaaagagaaaggaaaaagatagggtgggaaagaggcagcgggacagtgtctgagtccagcacacgccagggcgagccaggtcaactgcagctgtcatattctaactgtgaattatcatctttgatcactgccctttgagatgccaatgaacttttcaagaaatatctagttctcttggctctccagctgttcttatcagccccatccaggatggaacagctttggcagcccgtatcagaacaagcagcttgacaggggcatgccatgccaggagagaggatcctaaggaagcgtggtccagtccgcacaggctctggggctttaagataaaacctcctgtctaactttagtaggactttctgttgcttcacctgccagagccctgaacgagggataaattgacttaattaactagaacacactgcaaatggtgaaagcatttagcaaaacaaagaatgccatccaagccccaaaataaaagcagaataaatagaatgcaataaacagcaaccatcccaaactgagttctcagcagcaaatctccagtatgaaattttggattttgtgcgtgtgtgcttaaaggtggatgacaatgacagttcatgggattgagctctggggtccagagttggcatctgttcatttcccattttgtcattttacccttgattgactgaatgtcagtgccttaactttgggctgtggagtgagtcggaactcccccgaggtgtgcaggtggttgttagagtctcatttttgcagggtggaagacaggagggctgcagccttcattccacactgacatggtcattgccgtgtgttctgggtccagatcaggcatattgacctgacatatgacctgacaacaggaccactcagaaagtccagcatgcgggatatgatttggagagccagtgggggaaatcataggtcctttctctgcatgtgtattcaggcaatgtcccagggctgggcggcttccgcattgcttggatatcggaaaatgcaaaaatgcccctgaagactgagacttcagtcttcaaaatgaatgtttgggaaagaaagttaacggcactgctgtacttgtggtattcattgcattattttattttggctttcagcttaaagagcaaattctgggtcaatgaacagaggtaagaaactatttttatcagaattaaaatctcagattgattcattgttgaaataattgcacacttttaaaaggcacacctcacagccatgaggaggggctgttctgtaggtgctcaggaagtcacaagacacgtcctgaagaatatgtggctagggacatcccagactcagaagacactcagtggtgcctcttcttggaggacataagtgggggtggcattccctgatgtggcgtttcagagcattctcacccaaaaaaagcttctaaaacctccaagtatataacagtttataatactccaacaagagggccttgtagcctaaacccgggacactccttggcccattccttttaagcttcagggagtgtgggccagccccagactcaccccattcctgaggcatcctggaggttgaaatatttccagaggtttagaacctcaccaagtgggactctaggagcctgctgcctcccagcctccctcaggaactgcacctccagaacaggtgcggggctgacatgtatgtgctttcctgggcagattctagaccgtacacatgaaatctggctttcaggattgctctccagagggacctgtggggcctcggctgagacagagagtaggagtgaggcagtgattcaaggccctgagaaagagctcctcctctgcttggtataaccagctaattcattctgttctgttgactttggcttctgccctgcctttgaagggtttgaggccagggagtgatgcactcagactggtgtttccacacagtcacttcagacttccagggcagtacaggagatagatcccagggccagtgaagaagcagagcacaagtccaggcaggagaggctaagggcctccctgaacaggtgtgaggcacagaagccccgagaggtagggatgacaggatgaagatgggtcctgtgctgctagaagtacctgcaaagcacagaggtggcacagaaaaggagtccttggctgggatgggaggagatgacatgtgacatgtgaaagaggacctggagttggctcgatgctcccaaaagggaaaggtgccgaggggagctagcagccatgcaaaggcagagacatgcaggcagtctgggccatgaggagctctggaagtgactcgatatgtccagaataggccactccagggaagggctgaggaaggatgaagttggagaggggcacagaccagatgcagaagggcctcagaggccaggatgagggtttggactccttcctggaggcagcagcagtgggaaaagagttaaaagctggtttgtaaagtggagccatgttgctcgctggtccaggcaattcccccgaaagttcatgtttccctacaaaacccgagagagctactagtaggcgtgaagttcgtggccctggtctgaggatttcctgtttccttgtcaggtatggaggaatttccattggaggaaagctcccagtcgtccccatcacgggggaagcacttgttgggtttttaagcgaccttggccggatcatgaatgtgagcggggtatgtaaacagactggagatttgagtaggatttttgacttgcttaactaccatgaatgagaaactctcatgagtgataacaggaaaaaaaaattaaaaccgtcttgtttgtttgtttacatggtttttagggccctatcactagagaggcctctaaagaaatacctgatttccttaaacatctagaaactgaagacaacattaaggtacttgacctatgtataatctgctctggagctaaaaatttacctgagctggttattttatttttactttcctaccttcattaaattccatccctcctcctgctgaaatctagcaaggaatgtcttccagctaccaaacccttcctgcttctcaaatttcctttccttcactgatttctgctttaactagctgttagtgcagcgtctcagatgtcctctccaccctctaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgtaagccaccacagccccagcctcaccactttcttgtcaccttctccactctttgaacatcctgagaggattctcaccaccgcgaagtgctgatttggatggtaatgctgtttagtcaggcacatatgaacatccgactttcaaataagtgcctcacacttcacataccagacctcttggtcattctttctccccaacatttatgtggcaagtaagtttacatttggttccattcccttttggcttttgatagcaagttgctcctggagcttatacaattattatctttgctatgtgcaaagcagctgccaggaactggcaaagttcagtaaacctttcagctccctcggagtaattatcttagattccaggaatttcctcagaagagcatactttggagatgtcgacagagctttgctaccctcaagctgaggctcttcttgcacagtttcagccagtggagacagtggccttgtgcgttttgtagtatgttcactctatttgaggcctacatggaggaggggttggtaggagcacctttgttagtgcaaacttcagcaacgttgtggggtcctgattttactatcctagcacacgctgagtgccagtgaacatgcccagggtcatccactaaaacctgggccttggctccttggtgtcttcctctggacaccctagggccctagactgtcctctgttaattctcactcagccacactttcgtgtgtctccttccagtcatttgttctaagcttactacgtgtatggatgatatgatctgtagttttatcaaggtagtgactaccacataggatacctttgtggaaattagtaaaaatgctcttttctgcaggtggacactgtcccatgccaggggttatggcttgtacataaagttcaggctggctttagccccaacttacccctcagccagatgccttctatttgtccgaggaaagaataaatagagccaagtccctgtacaacttgcctgccctcttttcacttaaatttacatcatgaacatttccttgtgttacgatgtacttcttgaaaatgtgatttaacaagatgattattaacaaaagataaatctcacagaccgtatgtctgtcaacatagaaaattcaagagactctatagacagattattagagctaatgagagcattgcagtacataagattaatataaacatctatttctatacaccataaaaataattagagaatataataaaaagaaaggttgtctagaaatattcacatgaaatagaaaggcaacccgcaaatacccatttaaccttggtccatatggattaagacagtttagtggagtgacagcttcaaggtagagaagaggaacctggaggccacacctgggcgggtgtaaggccttcccaaagcctgactttgtatcttctcctccttctgctcttccctcttcatcgccctctccctgtgtctctggccctgctgcaggctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcgtaagtgtcagtttacagcgcctccctcccctccgtgggcccaaggtggagcttgtgtgtgctctgaaggaccagaccaagaggggaggggttctcacggtgccagggctgctgaaaggcactgggccaagggccttgtgtatctgctgtcccttgacatcttctcagaaaggcacagaactaggagcccgaagctaggaaaggctgtggggtgcagcttaacaactggtgaacgggggctctctatgtcctgcactgaggggtcttctgacccatcaaataatcactgcaccgcaggcatgagtctggccttcctggcatcagtctggcgctgagaaggtaatatgaaggggtctttcaccccaagtccccttctcaaatcctgccccaccttcaaaagggtaaaggtaaaactttccctgtggtagggtcaccagataaatacaggacacccagttaaatttaatttcagatgatgaataatttttagtataagcatatgctacttcaaatattgcacaggacatatctacactaaaaaaaaaaaaaaaaaaaaaaaaaacctggttgtttatctgaaactcaaatttcactaggcatcctagatttttatttgccaaatctggcaaccccagccagtggccaaaataataagaccttcacttattagattaaccaccgctacagggaaaaatgaagaaaaaatatttattaaatcaatagcacactaccaccttcctgacaaccaaggttggtgggggtagggaggggtcaggatagcgtaccctattacaggctgcagggtcaaaggaattggtagtaaaggcctagttataatgtaacagggatcattatgacatcaaccccaatttattctaggtgtcttgagtagtaaaatctcaacattttaagaccaacatgagcctccatttcatgtgatgataagatataccaactgatggagaccaacacaaatgaccttctcatccatggttttttaaaatgatggtgaatattggaattcctgaagatatgatttctatcttactcagcttagtaagcagctatcacttaacaatacaaaaccagagattatcagtagcaactaaattatttcctctctcttctgtctacacgaggaaacactcataaatgcacggggaggaggtcagaacctgaaagcctttctttggataagagcatcaactgcaggtaccacattggccctgtgatgctaatataaaaggagctaggcccaccggtaccgaaaagttacttagaaaagtgcggaggcttttaattttactttttttaaaagataagaaatagaatttacacacttggggctggcccacgtgtttctgtgtgtgtgtatgtgtgcacgcacgcgcgtgtgcgcttacagggatctctgagcctatggagagagatgtagctaggatagagtggacatctgaggtgggaggtgatactagctggcagtccaatgaaggggtagaagatggtaggcatcatgttagcaggctttctgatgctccagaattttaaagctggcctggaatctcacctccgcgatccatcattttggaacttaggaccaccattagccagtggcaaaaaaaaagttgaatgaaggaacaaacaattattgcttatgtaattcacttagcacatatatgatgttttaaattcttatatgtgtcatctatttttctttactttaaaattttgcaacagttacagacttatggaaaagtcacaagtacagttgaaacctttttttcttagtcatttgaaagtaacttctcagcaagatgccccttctcatttatttctctcttcctgtctctctctctcacacccctcagcacgtccgatgtatacttcctacaaacgaggatacaccccatacaaccacaacacaaactgtcaacatgaggaaaccagcactgatgtgtcatcaccacctaatcctcacaccccactcctctttcgcccattgccccagtgatgtctttcagaaaaaaggatctagctcagaatcatgcatgacatttgattgtgctgtttctttagtctcgttcagcctggaagagttccacagtcttttgttaacactcatggtcttgacactttgaggactgcaggctggttattttgcagaatgtcccttggtctgagcttgtctgaggtttcctcttgcccaggttgagggtgtgcatcttggcagcagtatcagcaaacagatgctgtgttctcactgcatcctatcaggtggcttctgatttcaatttgctctgttactgatgatgttcaattcggtcacttaagaaggtgtctgctgagcttcttcactgtaaaattactcttttcccctttataataaatacaaatttcaggtagaggcacttcaaagatatataaatatcctattcattatacaattttccatttattcatccatttatttatctctgtatgcagtcatggttcatgtgttaatcaatggactatgatccaagactatcattatttattttgatattcacattatccccactgtggtcagtggggggccgttgaagctggcttctgtatcgtcttgacttgggtcctcatgcccctggacctcctccatgctcaatggcacagcaagatattccaggctcatccttccattatccccattcctaccctctccccaagaagccctggttcctgccagtgggaagtggccctcagaagccaaggtctgagtgctagatatgttcattgcctctggagcaccattggtcccaggccttctcagtgatagaactagggaagatatggatgtacacacacaggtatgcacacacctctatctatagttctctatctacctatacagtgaacactatgagctctccaaaaccaactccacagggctcattctagttttttttctttccacatctgtaactcccttctccaacagtgagacgctggcttctctcactcccaactcatttatctaccggacctatacacctgaacagtgcccaactctgccaccatcccctccccatgtggatgccgtcctctccctgctccagctgcctctgctgcatgcaggtcctcctcgttctgctctggctctgataccctgcaccagatcagcctcctgtaaggatatctttctcatcccgttgaggcctccacaccccacggcaggttgccccctgaggaagcccgtctctggttcttgccctgctcctgatcaccatggctcctcccctaaccccactgttgccgtcccctttctgtgcccagtatagtggctgtaggactaaattgtttaaaaagggtatcattatttatttgagctttgtgaagccaagaactaggctttaagtttttctgaattctgaagacatgcttagaaagaagaatcaacaaaactttatgaccaaatagaaagagtgagagaccaggcagaattttgtaattgatcctttcaaaagatacaaactaaaggttcccttggcagggaggtagggcatggggtggggtaggaggactagtgacagcttaacatatgtttgccaaccaagaactgtttaaaaagcaagtcgaatcagaatcccagaccctacgagctggaggagcctggccccacccctcattttgcagagctggcagcaggtctgagaggttaagtgacttgctctcctcttctctttccgagatgaattattccgtgagtgctgggctggtggtgggcatcttcatcgggtttcagaagaaagcctacacttctccagaaaaccttcctgcccttgtggcactgctcctgctgtatgggtaagccgtttgggccattagctaatgcctctgaagagaagcctggtggtgggggtgggggatcatctcctgacagaaaacctgggctgtcctgtggtggtagcacccacaagtttagcttccggccccaggtagggtctgaagctgataaccagggatctgtctggcttctgattctgactccactgacagaggtatctctgaggcctggtcctgtcagtgacaatgagagaagtcccacatgatctgaatctcctactcaaactgaggccttgaccaaagcctgggggcagccattccccaacccctcacccagctctgactctcactcatctgtggccaatctgtccacctcagtgtccccatgtgaactggccaagagttaccgcccacagtagaagactccggccaaaaagctcctcctgagtcagggacagaggatgacacaggggttacatcagcagagttacagggcccagcatgcaactttctttcccacgtgtgtaaatttgaatgagtaattcatccatctcggcctcagtttcctcatctgtaaaagaaaatagtgatcctggtccttcctctgtgggccagtagagccttgccaaagcattgttctccacatctttctcttggaaatagagaatttgggaaccaacctgactataagctgtgaagatgagctcactgggctcatctgagatgacctcagctgggctttgctgacccaggctagagtgggaggtgttgcaggctggagaaccctcctatgaattgtacagggctttgtagtttacagagtatatacacagctagcagcccatttgctcctcacaaaaccccatgaagtggtcaaggcaggcatcattatctccatttaaagttgaggcacagagaccaacaaatggagtatctctctggtcccctgggactctggccagttcacacacatcacctcaggtgtaaggggagtgcattatatccagacgtattgtaggtggaatggaatgtggaactccatcactctgagttgtctcatttcacacagatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccgggtgagcataactttcttggcttttttgtttgattagtaggatagtagagtatgtgttggtcgagcagagccaggggcaagcatcgtacatgtagcagctgtatgcggatgagtgccactttcttcctccctacccccgaccctgcctcctttccttccttccttcctcccatccttccttcctctttccttcttctcctccctcctccctccttcccccgtccctccttccttcctttttcattgcttccttccttccttcgtccctccttcccttcctctttccttctgccctctctccctttttcctttcatcctccctccatccctccctccatccttccttctttcttccttctttccttcctataagcacctttttcatttctgtgctctgaatgaaatggttttctgtgtttattctgcaagcaaaacttgattcttgcaataaactttaagctttgcttactctttcagaaaggttttctcagggactttgggtgttgggttttacacacacacacatcaatacatttgggtaatttcaaaatctaaaaggaacaaaaaggcatacaatgaaaaaatctccttcctacccctgtttcccactcatgcagttctcttctccagaggcaaactcttacttgagtttcctgtgtgctctggagacacatcagcagatccctatacggtctttctcccgctttcttatggaaattgtaacactctgacatatactattccttgggcaagttaatcttgatgaagagactgggtgttctccatgctgaatgcctcacttttatgagctgccaagcccagttgtcccttccacctgacctccccctgtccagagacagatggccaaactgaatcataaaaagagggggaaaaaaagaaggcagtcgctgcagggctgtctttactccacactccacactcccagtccccaccgctgtgtctgagtcctggctgtggctgtccttggaacatttgcctcaccacgtgcctgtgtccccaggcgcctcaacctttcctctcctcattagctcttcccagttcagagggtgggaccggccagcacatctgcactgctgccctgccacacccacctccacctgcctctgggccccactggggaacacaggacaaatctgtgcggaggccccaccatgaaccgcccagacccgtggacccctgagactgactctttccagatcttgttagggtttcgtggctgctaggcaagtaacgaagcctcatctgtcccatgaatgataagaaattcagcatgtcagagtcagactctggaaaggcggggggataagaacacagccccagcagatggccagagcacccaggtgactgaaagtgctgctttgcagagctgtgtttgccacaggctcacagcccactaagtcttaagacagttttccttcagaataattaaatagccagcttaaagcaactcagaacattttcccctctgaggctgcacccatttagccaacatttgctaagcacccgccttcaaaaacctggtattttcatgtaaattatccgatacacagctgctatggaaacccccagtatcccacaggaagctccccagctcccagcagctgccggcccgtgtgagatcaggaggtctttaccagctgaacaccacgtgccgggtgtgtgctgatataaacaagcgtggcccactcgtcctgccctccagaggctcccgttccagtcggaaaaggacctgcccacgaagtttgcaacgatataagccacagtgtatgatcctccataatacagcgtgtgacagagcagcagaggagcgaggcagataacatgctgcaggccagaggcagcgggaagagccaggctgcaggggctgggggagccgtggtggaggaagttcaatttcagcctgtagatttctattagcccatttaataaataatgaagtgcctactctgagctaatcattgtgcaggtatttaggaaggacaaaaaaataattaggactcagtgcccaccctccaggggcccactgactagtagagaaagtaggcagatttttaaaaaattaatcatgggaatgtgataagtgctgggagagaggaatggatactttctcatgggaatcttggaaggcttgtaagggaaggcactctctgagccagctgtctaaagaagaacaggaatctttaagaaagcagaagggaaaagagcattctttcctgcttggagcaataggtaacagcctgcacatgcccaggcctagaggccaaagagcacagtgattccagaaagagtggggagaaagggtaggcagggaaggatgaggtaatgtgggcgcaggtgtggaggctggagagggaggaggttgtgggactgggaggagccagatggaatggacagcagtggcccagccaggagctatgctggcctcgtacgcctcgatgtcccttctattttctcaggggaggctctgcccaacatgccaagtccgaccacttgaaaacaagtccctggcttaacacagaccccagagagagtctccaaccctcctctccctagacaatggtagttgccctgtgaggggctgaaaagcagagctggagatggctcagggcctggtgttaacaaatgccttgagggctcctgttgtttcaaagtgagtctgcagggagagctccctaagtggacagcaggagggctgcagcttctctgcacattcctgctgtcacccccagagtcacctaggggaggggtaaggacagtaatgcaggttcctcacagttagcctcggtgcccacatggtactgagcatagtaaatgtttagaagatgctgcctggctagacaaaggggaagctcccgcccactagaaacttgcagggagccccagtccttgattggtcatttaattgattagctccttggcctggccttgaggcactgcttgtaagtacttcatgacctccattgcaaacccatgatgctctgctggacaaatccctccagtggccagtctggctgcaaggactctctgtctgcaggccttgccctgtgctgtcctgtgagagcatctgggccccacctgctgaagagagggggggtggggtttgccccgtttccaacagtcctacttctctgtttcagacgctgctcaggttcaacgccgtgctgaggaagctgctcattgtcttcccccacttctgcctgggccggggcctcattgaccttgcactgagccaggctgtgacagatgtctatgcccggtttggtgggtggtagccgaggcccatggagcatgggccctgggtccaaagctgggagggttaccgggggggctcctgcatcagactgtggcaggggctggtgctaggaggggaccttgttgggctggaggtgtcctgccagctggagaggattagggtgcctctgtttccatggctggggagccacaggagggatggagggcagcccttatgaggcgggtgtttggctcttgctcagttcccacataaggcctggtctagtgggccctgtgctgtggccaggtctgtggggtgagctggggcggctgaagtggactcaattcctgttgatgcccaggtgaggagcactctgcaaatccgttccactgggacctgattgggaagaacctgtttgccatggtggtggaaggggtggtgtacttcctcctgaccctgctggtccagcgccacttcttcctctcccaatggtacgtccatgccacaccctgggccagtgggcagctcagggcatccagaactggaccttatacccacatggtcatttctttcctcaggagccccactccacaatgttttttctacattctcaaagcctggcttttctccaataatacaagtagaggatcgggttaaaataggcacattcaaatatgtgaagagcatccactttaaaatatttaaaatgcagtgctattaatttcaattgctgatatttaatccttctcatttaattaccaaatgtgtattttgattagatgatagtattgcaaataacaatggttacagggtatccaaagtactaggaaatagactaatgtatttatgagagaaaggacacagcaggcccctttgctaattagagatttgggagcatgggagtaatatgggagccatgtggaggggtgcgggcagtgatcacgaccccccactcctggaggaaggtgggtagctgccaaccctgacttttgaccagggcttctcaaatgccaggttagctggcaattgccattcttccgcaggctcttcctgaagctgggtgggcccctgcctcactcccctctgcaatccagtcctacctttattgtcctcacccaggggcctgaattgccaagcagcagcccttcctagcaagctttccccaatagtgttttgtttcttaacttttcctcctctcaggctgagtgtggtcacctgtaaatagattccaaggacttggttttatgttttgatccacagggaattgatttattggaaatgaatctgcctttctactcacaggactgtgagaggtgaatgagatcacaggtgtcaacacacgcctgatgaaacaggatacacaagcagttctagttatgggagacagtgtcaggaattgttgtccttggcaccctcagcccctgcagaccctttctgcagccttggccataccttttagaggcttttgtgtgggagagagcaggtcaggaggttgactacccaaattgactcattagcttcaaactctgatgtcaacacatttgaatgagtcctgcctgctttagggcctaaagaggaccagagaagtacaccatagtccctggcttccagaaggtcagggagggtttcaaagaagaggctgtgtctttaagaatggggaagattccatttggtggggcaggaggaggagaacattgagggactggaaacacatgcggaggctgggagacgggaatgaccaataggactgggaaccagggggagatgccaattgctgacagaggagttagtgcaagaggtaagtgagaagggtaggtggggctggattgcagggctgtaactacagctgcagagggagggcttcaacctacagctgatggggaacaacagaaggttttgaggcatgaggtggcctgatgacaactctgttttggaaaggtggagttggcagggcagactggaggaagtgggaggctcggaggttagtaactaccccttactgagtgcttgctgtagaggaagcattttagtcctgacggtgatcccaggccctgagtctttactctgtgccaggcactgtgctgagttcatcttcagcacaatcctatgagacaggtattgttaccctcctcctcatcacatggttgaagtaggcaaggttcagagaggtccaatgcccaagatcacacatgaggaggccaggactggaacccaaggctgactctggacatgagcacctgacctctctacctaatgcctaatgcctctcctgctgggagccctttttagaatttaagtcttaaaggatggaagcccagaaggaagcagaagcaaggaagtggaagagaggtcccatggaaaggacagtgccaaggacactgtacagccagcccaatcctgaccccttttcttcatctaggattgccgagcccactaaggagcccattgttgatgaagatgatgatgtggctgaagaaagacaaagaattattactggtggaaataaaactgacatcttaaggctacatgaactaaccaaggtaagggaatgggtatgagtttggaggtgctggttagatccacagttggcatgatgttgccattttccttctatagaacaattgatatgcttatgcaagcaatttggttcccagttttatgtagggtcatcatccctgtgttataactcgtcttccaagagcatctaattccaatgtgtgttccctgctattcatctcgggcactgacacagggcctcagtgagaatcactccagctgagcatcattcccttttctgtgttctgtttctgcagagcatgggtcagcctcgagatgtctcagtactcaccacacctctgtgcctgcccatgtcaatatgtaacctcctagtgctggtagttttctcctaaaccatcctttgctctttgttccctcttcccctccttgctctcaccctgtctcagttctcagtccggtttcttcgtatcttgcagatttatccaggcacctccagcccagcagtggacaggctgtgtgtcggagttcgccctggagaggtgggtactctgcagaccacgtgtgaaaggcttccgaacatcagctcttgtgcctgcctctcctccccataaggcagagctattcaataggaacataatgccataatgcaagtcacatatgtaattttaaatcttccactagccacatgagaaaagtaaaaagaaaataggtaaaattaatttcattagtattttttattttactcaatataaccaaaatattatttcaaaatgtaattaatagaaaaccttattaatgaaatatttgacaatttctcgttgtttttaagtctttgaatctttacactcagggcccgtgtcaactgggacttagatgtgtttcaagtgcttagtagccacatatggctcgtggcctctgatggcagcccaggtctaaaattcctcccccagctcacacacacacttaccctggggcctgacattttagaccttcttgatctctagggccaggctagctctgtgttttctcctagtgctttggcctcctgggagtgaatggtgccggcaaaacaaccacattcaagatgctcactggggacaccacagtgacctcaggggatgccaccgtagcaggcaagaggtgagtatcctgctcctcctgtctcagggagtctctcacaggtcctgtgagaagaataggaagggtgatcatcagaccctatagtagggtggctctgaggccctgaaagatctgtacagagaaggaggcctcccagagagcatggcccaaaaagcccaacacatagacccaatggaaaagtgaactgaattgtgatagttaagagattcctctgttgggatggattcttggaaagacctgggaagcactaagtgtgtggttcttaatctcttagaggtcacggaaccttttaagcatctgatgaatatttgtagcctattcctataaaaatgcaccattgcttcccattacctccctccacacatttttacaaaacgtttcagggagtttactgagccccaggtcacatttatgatcctgcaggagctcttgaatcccaggttaagaacccctgtgatgaatgaagaatccttcctctgggttgagtttctagataggggctcatgcatgggcctttggggtagcctaacctgcattggctatttgtaggctgatatttggctttgccagaccaaggagcatagagggaaaactggcgtgtgcccttggattctggagggtgactgctgctctctgtaataaaatgtgtttaaacagactggtcccctatgggcaggacagagaggatgagctctcactcatctgcctctttcctggctgcaggaaaagcttgaacagtaaaacttcagcacacacaatagaggtgcccagaggaagcctctgccctggtttataagtggagttaggtgctgctgacatctgtccagcatctgcttgactggggcctcttcctctctcctgaaagccatcctcagcatggcccaatgcccagtgggcaggacgagtcctgagcacgcttcactggctcagacaggatgaatttgattctttggcctccatagccagccctactgggtttacagaaaagggacaggcaggggtgaagccaggtcatggctgagtccatctcaacagatccagcttcacctgcaagtgaccacgcaggtgacttcctcatggtgacaaaaggagtcatggcagggtagagatatcataccatggcaggggaaagatatcatagaattttccatgagcacatttatgagacatcaagttacaactgtgtccaagtgaggcacagtctgacatccagaaggtaaaactgagctggacgctagaaagaaactataggcttaagacacagaattgggattatatggtagggtagctcccactaatttggaaacgtaccctacttgcttccctgagtagttttaattggcccagccatgcctttggtggcttttgtcattgtggggaactgtaatggtctctctgtaccatcctatatcatccatcctttattcatagaccctaagctataagaagaaaaggatgagattagactaaatgtctatgtatagtttattttccatcttggcaatatattttttagtgggggtgaatatattagccaaagggagttggtggaacccaactcactctacccctgctccctgcaggcctctcgctgtgggtagttatctgactggctcctctttcattgctatctttgccaataaatacagatagagaagtttacttccatcgggacacatgcatcttttctagttacttcccaaatgtctgaaaattattgataaatcatgaatcattttcttaaacctgatcttccctctgtttttaaactcacatgtgaggtgatctgatccaaaatgaaagctgacttttggcgtaacagggattcaattaatcctagacatggaaacatggaagaatctgacaggattcagtttctaaccgaagggcccctgttttgattcccaaatatcccatgcatttctgaagccaaataggagaagagaagaagcagcttccttttcccgttggcagaagcttctccagccctagctctatggtcatccctccactccttgaaggatactcagtaattgctttttttcttgcagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggtgaaaaatgttttgttgtggccacataggagtctggttaattacaagcctgtttcatgagagtgcattctcttggagatgagaaactgaagcgtgctattcattcattcattccaacaaatgtttactatgtgtctactgtgtgccaagtactgttctagaaaccaggagtatagcagtgaacaagacagacaaaaaaaaatccccactctcatatctaacaaaatgttgtatgcatttatcctctgactcagcaatcacacgtctaagagtttatcctgaagatgcatctcccacagtgcaaaatgaatatgtataaggtgatccattgcatttgtaattgcaaaatgctggaagttacctaaatgtttagtcattgtagattggctgaataatttatggtacagacacacaataaagtcttacgcaactataaaaaagaagaagaaaagtctcagtaaactgatatggagatatttccagtaaatactgttaaatgataaaaagcaaagtggaaaacagaacatagagaacgctactttgtatgtaagaaagaaggaaaaacaagaaagtaaacgtatgtctgcttacctttgcaaatagaacgtagaaaggataaaccagaaaacaatgaatttggtgatcaacaagaagaaaatgggaagaaagaaaaatgggaggaaacagtacttctggggatatatttttgtatagttttaatttttggaagcatgttaatgttccacatattcaaaaaaaatcagtaagaatgggaagtaggcaaaaatgaaaacaaaaagaaaacctaacactgacagcaaactaaataaagtaacccaattttatttcaaataaatatcataatcttgcaaaagggggatagagctaacacaaacaactgctgaacacagtgtttgactctatatcctcattcttgggcagggtggagcgggggagaagaactacaaataatttctgagttctttttagtttgttttttatagtggtataggcaaagtgattctgaaaattttagatgtgttacaggattaaataaattaataaatgttttgatgttattgggacccagaattctcaccgtggaagaagggacttacaaatatggaaaagggaaaagcaagaaagaactgtgaggtcatggataggaaccggaggtagcactgggaattcaggaatatttatatgcttgtgtttgtgggtgcatgcagatgtgttcatgtttcatgcacataggcatgtatatatagacatatatttgcatgtgtgtatctgtcttccgaaaggctcaagaagcaaaaacaccccagtagccatgagcacacttagcactcaggcttttgtcttaataacattccccactaaaagtaaccctgattcctccaataaatgataagttccagggctggaatggcataggtataaaatgaacctggaatatcttatgccagaaagtaaggaagtgcttttaaaaaaaaaataaggggctgggcatggtggctcacacctgtaatcgcagcactttgggaggccaaggtaggaagatcgcttgagcccaggagttccagattagcctgtgcaacatagggagaccctgtctctacaaaaaattagcaaacaaattagctgggcctggtggtgcacgcctatagtcccagctactcaggtggctgaggtgggaggaatgcttgagcccaggaggttgaggctgcagtgagctgtgatcaagccactgctctccagcctgggaaacagagcaagactctgtctcttaaaataataataatataattttaaagaaataaaagtaactctgtacagattgcttattggttacatgggagaaacataataattttacaatggagaaattagacagcaccttaactgggtgatcaaaattaaccataaggggcagatggacatctcatgccccgagatgtgataccctgtgaaggacacaatttcacttatgtagaatccagattggagatatgtaacctgaatcttatcatgaggaaacatctgacaagctccaaagaaggaatattccttaaaaaaaaaaaaaggagactgtattcttcaaaaacataagagtcataaaagacaaagaaagagctatggaaatatctctgatcgcaggaggctaaacaggcataatgactgaatagcagacaatagactacatcttgtgcagaagagaaaaaaaatgatagaaggatattattggaccaactgacaaaactgaactatgaacagtagattaggtaaatgtatcataacattaagtttactgacattgataatgtactgtggttatgtaagagaagatctctattcttaggaaatatgccctgaagtatttaggagtgaagggctgtgatgagtaatttaccctcaaatgggtcacaaaaaattgtgtgtgagagagagaagggttttattagttaataattctatgaactatttttattcctatatgtttgtgtgagtttgaaactatttccaaataaaaagttaaaaatggagattacattctagtgggagggatagacgatctgtagataaataggtaaaatatccagtacattagagagtgaaaagtcctcagggaaaagtaacgcagggaggaactgctggggcagggtttgcattttgaggtagggtggcccagggagagcctgcagaggagagaacctgaatgaagaactagaggtgagagaaggagccacgtgcacacctagggaggaacattccaggcacgggggactagtatagaaggcagaagcatggtgagcttgtctccagtggcttccctagatcccctcctgcgcatgtgcacacacacctggtgtctctgtcatcgttccctcacagcactgtcacgatctgccagtattctgtttattttgactgccacctccccgcagtctgaggatagcagcaatggctgtgttcacattgttctccagtgcctggttcagtgcctggcgtatggtcagtgctccataggtatgtgtcggatgcacaaggctttgggtgtaaccctcttgacgggtgggatcaacaggtctgggactcaccatcttctcaaacagagccttcctcctccactgctagccatggtccaggacgctgggcgagacccactgtcttgctctttgtaaggctgaagtccatttcccaggcggctacacccaacagatgctgagcaggctgggccaccctgggatccaagacacagagagaaagagcccctgtctggcgcctgaagcacatgccagaggacaggagccagcaggagcctgtttcagcctagctggggatttcattctggaggcgtgagatctgggagcccaaggctttgaactgggggaggtttggggtgtttgcttgtcttctccaaatggcatttctttctcttccctaggttgcaaactggagtattaagagcctgggcctgactgtctacgccgactgcctggctggcacgtacagtgggggcaacaagcggaaactctccacagccatcgcactcattggctgcccaccgctggtgctgctggtaactgcgggcttgggccgcaccaagggcttaaaccaagtgctgggtctcttgggttggggaaataggttctgggtcggcagatttagaaactgcagcagtttggctttagtctggactgtttcctgtgttgctcattttgagcgatcagcccagtgtttggttcacacagctccggagaaaaacaagtcacggcacagccttgacttgggactgcgcacatcctgcgttcccaggatgtctcctgtggggccatcggctcacagccgggaagttcagcccactctgcggcctgtcggtgtctggtccccatacaggagcactgagctgggtcaaaggctcctgagctgagccaggccaggcctgaggccatgcccacgcagcccaaggatcatgagggcacaggacatagcgggaaccaaggaagtgacctgagtgacctccctgccttctgacaaatgtatttgcaggattttctttttttgaggagaattctgtcattgccttaatccactttaatcccctcgtgggctgaaatgggcccaggatggacgccacgcttctttactcttggatccacctcctgccttccctaccctacaccagggtacccctgtcttgctcaagtgaggggagtgactgtgtgcgccttctgtcagctcatcctccacaggggagccagcccagggggaagcagtaatcagaagggccagctcccagcctgtgcccccaaccttctctccaccccccaggatgagcccaccacagggatggacccccaggcacgccgcatgctgtggaacgtcatcgtgagcatcatcagagaagggagggctgtggtcctcacatcccacaggcaagagattcccagggctggggaaggtgggtgggaatcctctcctgctcacctcctctctcctgccccacagcatggaagaatgtgaggcactgtgtacccggctggccatcatggtaaagggcgcctttcgatgtatgggcaccattcagcatctcaagtccaagtaagcagatggtggggcgtgccccttgttgccttctgtggatccacctggatcctgtgttctccattgacacttggaagagtcctgctgctccgtcatcccctggggcagaggcaggtggtggctgggcctcattctccagcagcagatggagaaggccatcatgctgataagaaactcctctatattggcctaatttcctgtggtcgaagactcgcccaagtctctggatggggcatctgatcaggatgcatgcagagcctggctgggatgagggagggctgctaccactgcctcaatatttcaccacttatctcaacagatccgggacctgtggcctatttactaagagtccactccaatgtaggaatggttaggagaccaactgacttgaggacccatctttgtttttagaatattgtatgcttttgagtttgaaaaaagaccatatgttatatgacaaaccaacaatggcagtaatcttgaataggattatccttatcctgtacccacacattgtaaactattgtagataattccttattattaagagtttgcatgccaaagctaacagtttaagattatcagcatattgccgtgctcattcacgttctgatatgctttataacctagaaaagagcagagttacaattactcatttatttaacaaacacttattaagagctcagaatataagtcactaagctggttggtgggaggaacagcacataacccaccttatctatgctgaggtgcataatcctgatgcacccacaggagggtgttacacagaagatgtcatcctttcatatgtgtcagagcagataaataattgagagaaaggtctaatagattagctgcttgtggcaagtggacgtttgacccatgatttattgagcaactacaacttggacactgcatagatatctatagaaatagcagcatgtcaggtcaccagacctgtgtcagcaacttcctgtgtccaactgctggagaaagggaagtctcctattcctttccctccagctccttaatatctccatgatagagggggtgagaggggagtgttccctgtgtggagggatggtgagttttctggagctgaaaggtaaacagcctttctcctctgcatcttactgcagaggagaacagccctagactgtggaggaagctttggagtcagttatgactgacacaggataccagggcatagggtactgacacccgctagccgtgcacacactctctggtggaccatcactcatccaagagagggtaaccagccatcctgctgaaggagaaagaaagcaccaatggcccaagccctagcagctccattgtttcaggaagcttcctcagggaagtgctgccttcccgagcctttgctcccacctggcccatcagcccttaccaccactcagtatgcactggtccacgtgtctttatgggcagtcttgggatccccacactgggctaaaactacctttgacggccaggtgcagtggcttacacctgtaatcctatcactttgggaagctgaggcaggtggatcacttgaggtcaggagttcgagaccagcctggccaacacggtgaaaccctgtctctactaaaaatacaaaaattagatgggcatggtggtatgcacctgtaatcccacctactcgggaaactgaggcacaagaattgcttgaactcagaaggcagaggttgcagtgaatcgagatcacaccactgcactccagcctgggtgaaacagcaagactctgtctcaaaaaataaaataggctgggcgtggtggctcatgcctgtaatcccagcactttgggaggccaaggcgggcggatcacttgaggtcaggagtttaagaccagcctggccaacatagtgaaaccctgtctctactaaaaatacaaaaaaaaaaaaaaaaattagccgagtgtggtggcaggtgcctgtagttccagcctctcaggagactgaggcaggagaattgcttgaacccaggaggcggaggttgcagtgagccaagatcatgccactgtactccagcctgggcaacggtgagactgtctcaaataaaataaaataaaataaaataaaataaaataaaataaaataaataaaataaaataaataaaactacctttgacttcagcaagtacgattatcccacattaccatgcagacatttgatctctaaaaactggtatcaaatgatttctccagggactaccatggtttttctctcctagttttcagtatgtacacaggtctatggtatgggcctttaatccccagtatttctttttttgttgttcttgtttgggtttgtttcttgtttttcggtttttttgagacagggtctcactctgtcacccaggctggagtgcagtggcatgatcatggctcactgtagccttgacctcctatgctcaagtgatcctcccgcctcagcctcccaagtagctgggaccacaggcatgtgccaccatgccctgctaattttcgtagagacagggtctttcttgttgcccaggcttatcttacattcctgagctcaagtgatcctcccacctctacctcccaaattgctgggatttcaggtgtgagccaccaagctgagcttaatccccaaaatttctgatgagtctactccttattttgggattaccttaggcccaaccactaacagaggcctgtcctgcactgtgtgcatcccctagatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggcaagttggccctggggcaccgagagctgagcaaagactggtccagaacacccagtgtgggttggaattgccataagagggaggcataacattcccgatttttaacaaactcttgccctctgtttattggggtaaaagctgatatatcagaaattgttttctaacaatattttttagtcatcaggaaacttcattgattcttttttttacattttccttccctgtgatgctatggtgtgttatttcattcttgctcgtttgtggtggtggtttttccttcaaatcagctttattgatgtgtaattaacatacgatgaaacacaggttctttgggaggccaaggcaggaggatcacttgagcccaggagtttaagacaggcccatgtaacaaagtgagactttgtctctacagaaaaaaaaaaaaaaaatcagaaaattagccaggcgtggtggtgcatgcctgtggtcccatctacatgggaggttgaggaaggaagattgctggagcccaggaggtcaaggctgcaatgagctgtgttcataccactgcactctagtctgggtgacagagcaagcccctgtctcaaaaaagcaaaacaaaacaaaaacacctattttaaatgtacagtttagtgagttttgataaacgtgcattccatgtgtggtttttaaaaatgtaatcacattttttattgcggtaaaatataataacataaaattgaccatgccaaccatgtttaagtgcacagtgcagtggcactaagtacatttacattgttgtgcaaccgttaccaccatccccgatagaactctttcatcttgcttcagtgaaaatctgtgcccattaaacactaactcaccacttactgcccccctcgcccttggcaactactgttctactttctgtctctaaggctctgactactatagatacctcatataagtggaatcatacagtgtttgtccttttgtgtctggcttattatgcgaggacttagcataatgtcctcaaggttcatccgtgttgtatcatgtgccagaatttccttcctttttcaggccgaataatattcctttgtacgtatatgtgctacattttgttcatccatctattcattcattgatagacatttgggttgtttctgggttttgtgtttttatatatgtttttttaaaaataaacatctttagagacagttcagtaaagcagtggaaacagggaagtctccatttaacccctgaggatctggctcacctgcaccttctcatcagcattaagcagagggaggcacgagcaggagccacctgcacactcaatgaggagctgaacagggatcaattacctttttttttagttattaggatgctgctagctgagaatctgccttgccttgattaccccaatgtctggtgcccaagtcccttgagtcctccagcaggaactcctgtggcatcactcaggagtctagtctaagaagctagctctgaccagggcagtggtggccaggcttctgtgagtgggccagcctcccccgggtaggacacaagccataccagcagggctgtatgtgaactgtggaaaatagagagcaaagtgggtaggtgggtgtagggtgctgttttcctggaaatatctacctaatctcgctcttctcttacctctaggtgtttgtaaattttgctaaacagcagactgaaagtcatgacctccctctgcaccctcgagctgctggagccagtcgacaagcccaggtacccctgctgcttatgcagtccacagcttgaggcagttccttggctcagagcccagctggttcactgggcttgagttgctccaaggctcagatatgcctcctacagagagccccacccacaccacggtccctaccaagtccccaccacatcctcatcacatccttgctaagtccctgccactgtgtgttctgtgctgaagaacttttcattcagtagttgtaggggttcctattgtaatcaggaaaccatctggatagcatgggagagcatttttgaaaagaactttcccatgtttttgcttacagcaaaaaagcttggatttggggaataaggagcagagaaggtaatagagaatattagaatgttttgggtgcttgacatctatgtctggacatgtgtttgagtttcaagggaagggacttaactggcacatcatttcagtgtcagacacatttggttagatcaaggaatagcatctgttgtaggaagagggctctttgttctttataaaaattacaagaagatggagaaagaagcaataggaggtatgtctcctggcttgtgataactcttggaataggtgcttgtaggttcctgccctggcacagtgccccatgtaaggagcacaccacccaagaaggagagagctagagcaagtactggaggaggcaccagcatcccaatgccttggcttaagcctgggattgtagagggatgaattagccactctcttctgacttacctggagagtaaatcaaatcaaatcaagaagcaaggatatgcaaaaaccttatttccccataaagtttttattctgcccagtttctggattgcaagaaaaaccaaatacagctaatgattgaaacactgctgtctaaagcagtgcttgtgatgaattttttcccttcctcttgaccagcagagacctaatggctacttggcaaaactgactttgtcttcccaccccttacctgccagagggcccagaaatgcctaaggctcctttagttacagaaagtttgcttttactgagatcttccagccactgattcccatttatagatctggtgattgctgttgacatcagttgaaaattatttttaaaaaccacttgcagttgcaaatcctttttataactctgtaactcagaatatagaattgggtagcaaaattgtttcccagaattaccaatggtctccccacccctgcctggcatgttccctcttaaaggactaatcccaccacatcacctctgggccaggcagaacatcaggggtgctgatgttctgtgatctacagcagttaattccaaacttttctcccttattggatgagatcatttttctattgtgttttttacatttttgttcacaaagattagaaaacctgcaacacacttattggcatatttttctgataattttcatccaaaacctaattctgactttacaacatactatctttacaaaggtttgcaaaaattctttcatatagcattgtatatgtctgtcatgaaataatagtaagtatattattgtttacattataccacttcaaaataatttcctttaaagtattcttcaaacaagaaaaaggcaatttctctcaagaagttttagagagaatttacaacttgctcctaagcaaatgtgagaacttcaggaggttcatctggccattggctttacaactccaaattgtgagccaggaccacacagatatttctctagaaatcagcgtttgcttaccaagaacatttttactctccaaaggactccatcctggaaaacatgttttgggataaggtcttatgcaatcttatactctgttattaaaaccagtgagggtcaaggtgttaatagattaagtagtgacagatgatcagacaacttagaaacatcctaaataggttaataattatgtgaccatcgcatgtgcattcccaaattaggaacaactcagatcaatttctaatccttattcttacactgttccagttcccccatataactcgtatctttgtgttagtttcagaagtttctgaagtaccctcagccttgatggggatcctcgcaccacctcaaatcctgttctcagccctaagaactgtgttagtcatcctcttaagaggatgtgtgattttaaatcagataatgggataaaccacatttcgtctagactggtcaggcctttgtccagtcccctcctcgcccacactaccccagctccacagcgggcattggttcaggaattcaacccacactttataactggagacagtatctctccagttaaaaaggtcaccttggtgtccgcttctcaaggaacatggacatctttattaatcaaagcccaagctttgatctggagcctaatatcctgcactccagctctcatctctcccctcccccagtcacactttcatgcttcccagagccacccctacaggaagtggtcaagggaattctatacctcagggctgacctaaattaggatttcttggcttttaagataatggtaactttcttaagctaaaaaagccccaaaagaccctgtaagagcccttggaaacagcaccatgggtgtagcttccccccaggatgtaagcatgtatgcacacatctcgtatgtgtgtctttgtaacaaatgcctggatcttagtaccagggagacctgattcatagatttcatagagaaggagagaaagatggcccataacctgggtgatctgacagaatcacagtgccctcagctgagtgcccttcagaaattgattgacaactgtttagcttttgaaatctaaaagtagtacagcatctcagaaaaccaagatgacgcgagtccatgtgatctccttccacaggactgatctttcacaccgctcgttcctgcagccagaaaggaactctgggcagctggaggcgcaggagcctgtgcccatatggtcatccaaatggactggccagcgtaaatgaccccactgcagcagaaaacaaacacacgaggagcatgcagcgaattcagaaagaggtctttcagaaggaaaccgaaactgacttgctcacctggaacacctgatggtgaaaccaaacaaatacaaaatccttctccagaccccagaactagaaaccccgggccatcccactagcagctttggcctccatattgctctcatttcaagcagatctgcttttctgcatgtttgtctgtgtgtctgcgttgtgtgtgattttcatggaaaaataaaatgcaaatgcactcatcacaaactatcctaattcacagtctccctggtgtgcaccacctagtatagttttagacattctttagatgggtgcatagctccttgtcagtcccatgcacttctgtgagtgttactgcctcaggactgctcgttctggcaagattctgcaacactaggttggaagtgaatggactagtcttaatgttccatgtcaagtctttgtagagtttgaagaaaacacccaactagtaatgcctgtaaacattatccattgtcagctgggtattactgactttaagtttcgtgtctgtttgcccagcttatttgagtgtttacctcacaagtgtaattaggacaggagacaaagaggcatgcacaggcgagagttggtccttggttggacgtgagacccgacaggactttgtaaccatttgaagaggtcaggacctcattctcatgctgctgtgccttttctgcagtgctaccatgtgcatcttctgcagtggtgttagaagggaatgaaggccgggcgcagtagctcacgcctgtaatcccagcactttgggagcctgaggtgggcagatcacaagatcagaagatcaagaccatcctgactaacacagtgagaccccgtgtctactaaaactatgaaaaattagctgagcatggtggcacatgcctgtagtcccagctactcaggaggctgaggcaggagaatcgcttgaacccaggaggcagaggttgcagtgagctgagattgtgccactgcacaccagcctggcgacagagcaagactccatctaaaaataaataaataaataaaaaggaatgaagatggacatctagttcacataaatgctcatcaagattcagaaaataataattttaaaccaaacatttccagagactgtggatgaggagaacaagtgggtttccttgtcagggcagtctcccctccatgacctagagatgggcttcgtcgtaaacatgctttcattatactgaggatagactccagagtcggggtgaggcaagaggaaaaggggagagatgctgaggcccaggaactgttgctgagaagaggatgagaaagaaagtgagcacaaggtgctaaaatttttgtttcagctgtgcttgtttagaggcagacagagggcaagggctacacaactttaagtcctgcacacctcctggcttgccactttgcaccttctagatgctaggcaaacaactgcccatagagactataaaactactttggtaaccccgcagcttcatctggtgttgactttttcttttaagttaccaaggaccaaaactgtaagaccattttagctagggctaggatagaggttgggaaagcccagcacactgcttgcatcacactgctgcaccctggctggtttcataatttaaattagcaactgcaatatcacaggaaagaagactacactcttcggggctgcttaggaaaagaaaactaaaaaaagactatgtaggggaggtggtttagcagccattctgtttggctgtgagggtttcggaaaggcatcatgaactgggaagagtcgatgcaggtaaaatctgcacacccccttaaggaaaaatctcagtttaccttttgtctccactacacctgaggtctgtgtcttttcatcctgttttttccagctcctggcacacagaaaatgttcaactaatacccaccaaactgaaaacccagcaaactatgaaaactcagcaagaaaaatagacagaaaagaagtgggtccaagaaaatgatgcctccaaaaagcagcaaagggcaagtggagcaggaggatgccgtgctttaaaaacagccacaggccgggtgtggtggctcacgtctaatcctagcactttgggaggccgaggcgggcggattgcctgagctcaggagttcgagaccagctggccaatgtggtgaaagcccgtctctattaaaatacaaaaaaaaaaaaaagaaagaaagaaaattagccaggcgtggtggtgggtgcctgt

SEQ ID NO: 17 - DNA, 7328 bp, Homo sapiens
ggacacagcgtccggagccagaggcgctcttaacggcgtttatgtcctttgctgtctgaggggcctcagctctgaccaatctggtcttcgtgtggtcattagcatgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgatctttcacaccgctcgttcctgcagccagaaaggaactctgggcagctggaggcgcaggagcctgtgcccatatggtcatccaaatggactggccagcgtaaatgaccccactgcagcagaaaacaaacacacgaggagcatgcagcgaattcagaaagaggtctttcagaaggaaaccgaaactgacttgctcacctggaacacctgatggtgaaaccaaacaaatacaaaatccttctccagaccccagaactagaaaccccgggccatcccactagcagctttggcctccatattgctctcatttcaagcagatctgcttttctgcatgtttgtctgtgtgtctgcgttgtgtgtgattttcatggaaaaataaaatgcaaatgcactcatcacaaacta

SEQ ID NO: 18 - protein, 2273 aa, Homo sapiens [sequence omitted]

SEQ ID NO: 19 - DNA, 8035 bp, Artificial Sequence
  Synthetic Construct
ctcttcggtatcacaggagaatttcagggagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacagggagtttaaacagataagtttgtacaaaaaagagaggtgccaccatgacagtgctggctcctgcttggagccctaacagctctctgctgctgcttctgctcctgctgagcccatgtctgagaggcacccctgactgctacttctctcacagccccatcagcagcaacttcaaagtgaagttccgcgagctgaccgaccatctgctgaaggactaccctgtgaccgtggccgtgaacctgcaggatgagaagcactgcaaggccctgtggtccctgttcctggctcagagatggatcgagcagctgaaaacagtggccggcagcaagatgcagaccctgctggaagatgtgaacaccgagatccacttcgtgaccagctgcaccttccagcctctgcctgagtgcctgagattcgtgcagaccaacatcagccaccttctcaaggacacatgcacccagctgctggccctgaagccttgtatcggcaaggcctgccagaacttctccagatgcctggaagtgcagtgccagcctgacagctctacactgctgcctccaagaagccctatcgctctggaagccacagagctgcctgagcctagacctagacagtgagcttccactggattgtacaattaccaacaataaaatatctttattttcattacatctgtgtgttggttttttgtgtgcgctactttcggtatcacaggagaatttcagggagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacagggagtttaaacagataagtttgtacaaaaaagagaggtgccaccatgtgccctcagaagctgaccatcagttggttcgccatcgtgctgctggtgtccccactgatggctatgtgggaactcgagaaggacgtgtacgtggtggaagtggactggacccctgatgctcctggcgagacagtgaacctgacctgcgacacacctgaagaggacgacatcacctggaccagcgatcagagacacggcgtgatcggctctggcaagaccctgacaattaccgtgaaagagttcctggacgccggccagtacacctgtcacaaaggcggagagacactgagccactctcatctgctgctgcacaagaaagagaacggcatctggtccaccgagatcctgaagaacttcaagaacaagaccttcctgaagtgcgaggcccctaactacagcggcagattcacctgtagctggctggtgcagagaaacatggacctgaagttcaacatcaagtcctccagcagcagccccgacagcagagctgtgacatgtggcatggctagcctgagcgccgagaaagtgacactggaccagagagactacgagaagtacagcgtgtcctgccaagaggacgtgacctgtcctaccgccgaggaaacactgcctatcgagctggccctggaagccagacagcagaacaaatacgagaactactctaccagcttcttcatccgggacatcatcaagcccgatcctccaaagaacctgcagatgaagcctctgaagaacagccaggtcgaggtgtcctgggagtaccctgacagctggtctacccctcacagctacttcagcctgaaattcttcgtgcggatccagcgcaagaaagaaaagatgaaggaaaccgaggaaggctgcaaccagaaaggcgctttcctggtggaaaagaccagcaccgaggtgcagtgcaaaggcggcaatgtctgtgtgcaggcccaggaccggtactacaacagcagctgtagcaagtgggcctgcgtgccatgcagagtcagatctggtggcggaggatctggcggaggtggaagcggcggaggcggatctagagtgattcctgtgtctggccctgccagatgcctgagccagtctagaaacctgctgaaaaccaccgacgacatggtcaagaccgccagagagaagctgaagcactactcctgcacagccgaggacatcgaccacgaggatatcaccagggaccagacaagcaccctgaaaacctgcctgcctctggaactgcataagaacgagagctgcctggccaccagagaaaccagctctaccacaagaggcagctgtctgcctcctcagaaaaccagcctgatgatgaccctgtgcctgggcagcatctacgaggatctgaagatgtaccagaccgagttccaggccatcaacgccgctctgcagaaccacaaccaccagcagatcatcctggacaagggcatgctggtggctatcgacgagctgatgcagagcctgaaccataacggcgagacactgcggcagaagcctccagttggagaggccgatccttacagagtgaagatgaagctgtgcatcctgctgcacgccttcagcaccagagtggtcaccatcaacagagtgatgggctacctgagcagcgcctgagcttccactggattgtacaattaccaacaataaaatatctttattttcattacatctgtgtgttggttttttgtgtgcgctatattcggtatcacaggagaatttcagggagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacagggagtttaaacagataagtttgtacaaaaaagagaggtgccaccatgagactgctgctgctgacattcctgggcgtgtgctgtctgacaccctgggttgtcgaaggcgtgggaacagaggtgctggaagagtccagctgcgtgaacctgcagacccagagactgcccgtgcagaagatcaagacctacatcatctgggagggcgccatgagagccgtgatcttcgtgacaaagagaggcctgaagatctgcgccgatcctgaggccaaatgggtcaaagccgccatcaagaccgtggacggcagagccagcaccagaaagaacatggccgagacagtgcctacaggcgcccagagatctaccagcacagccatcacactgaccggctgagcttccactggattgtacaattaccaacaataaaatatctttattttcattacatctgtgtgttggttttttgtgtgcgctcaaaaaa

(19) United States
(12) Patent Application Publication
(10) Pub. No.: US 2022/0305142 A1
(43) Pub. Date: Sep. 29, 2022

(54) OCULAR DELIVERY OF THERAPEUTIC AGENTS

(71) Applicant: Intergalactic Therapeutics, Inc., Brighton, MA (US)

(72) Inventors: Robert FARRA, Acton, MA (US); Jaka CEMAZAR, Belmont, MA (US); Gayathri RAMASWAMY, West Roxbury, MA (US); Cathleen GONZALES, Boston, MA (US); Rosario FERNANDEZ-GODINO, Somerville, MA (US)

(21) Appl. No.: 17/721,063

(22) Filed: Apr. 14, 2022

Related U.S. Application Data
(63) Continuation of application No. PCT/US2022/021209, filed on Mar. 21, 2022.
(60) Provisional application No. 63/316,699, filed on Mar. 4, 2022.
(60) Provisional application No. 63/293,297, filed on Dec. 23, 2021.
(60) Provisional application No. 63/167,463, filed on Mar. 29, 2021.
(60) Provisional application No. 63/167,296, filed on Mar. 29, 2021.
(60) Provisional application No. 63/167,437, filed on Mar. 29, 2021.
(60) Provisional application No. 63/163,350, filed on Mar. 19, 2021.

(51) Int. Cl. A61K 48/00; A61K 41/00; A61K 38/17; A61K 9/00; A61P 27/02; A61N 1/32

(52) U.S. Cl. CPC A61K 48/0075; A61K 41/0047; A61K 38/177; A61K 9/0019; A61K 9/0048; A61P 27/02; A61N 1/325

(57) ABSTRACT

The present invention involves devices and therapies for ocular diseases or disorders in individuals. Provided herein are therapeutic agents, and devices and methods of delivering therapeutic agents (e.g., nucleic acid vectors) to target ocular cells (e.g., retinal cells) involving methods of administering therapeutic agents to the individual and methods of electrotransfer of therapeutic agents.

### CROSS-REFERENCE TO RELATED APPLICATIONS

This application claims priority to International Patent Application No. PCT/US2022/021209, filed Mar. 21, 2022, U.S. Provisional Application No. 63/163,350, filed Mar. 19, 2021, U.S. Provisional Application No. 63/167,296, filed Mar. 29, 2021, U.S. Provisional Application No. 63/293,297, filed Dec. 23, 2021, U.S. Provisional Application No. 63/167,463, filed Mar. 29, 2021, U.S. Provisional Application No. 63/316,699, filed Mar. 4, 2022, and U.S. Provisional Application No. 63/167,437, filed Mar. 29, 2021, each of which is incorporated by reference in entirety.

### SEQUENCE LISTING

This application contains a Sequence Listing which has been filed electronically in ASCII format and is hereby incorporated by reference in its entirety. Said ASCII copy, created on Apr. 6, 2022, is named 51503-066005 Sequence Listing_4_6_22 ST25 and is 392,808 bytes in size.

### FIELD OF THE INVENTION

In general, the invention features ocular therapeutic agents and devices and methods for administrating therapeutic agents to ocular cells.

### BACKGROUND

Visual impairment and blindness constitute a major global health concern, impacting millions of patients suffering from a wide variety of ocular pathologies. Retinal dystrophies, for example, are chronic and progressive disorders of visual function, which occur due to genetic abnormalities of retinal cellular structures (e.g., photoreceptors and/or retinal epithelial cells) and visual cycle pathways (e.g., phototransduction and visual cycle pathways required to facilitate conversion of light energy into perceptible neuronal signals). Vision impairment caused by retinal dystrophies varies from poor peripheral or night vision to complete blindness, and severity usually increases with age. Due in part to complex biological mechanisms and restricted access to the retina, safe and effective treatments for many retinal dystrophies remain scarce.

Recent developments in gene therapy show potential in treating retinal dystrophies. However, current delivery modalities often rely on the tropism of virion particles, such as adeno-associated viral (AAV) vectors. Success of such delivery modalities is contingent on a variety of factors, such as target tissue location, route of administration of the vector, and host response. Additionally, AAV vectors are limited by size restraints of the therapeutic gene to be delivered, rendering such modalities unsuitable for delivery of many retinal genes. Thus, effective targeting of ocular cells remains a challenging endeavor, and improved approaches are needed for effective delivery of therapeutic agents to retinal cells.

### SUMMARY OF THE INVENTION

The present invention provides approaches for delivering therapeutic agents (e.g., nucleic acid vectors encoding therapeutic replacement proteins) to ocular cells (e.g., retinal cells). In some instances, approaches described herein involve electrotransfer, a process in which transmission of an electric field into an ocular tissue (e.g., retina) using an intra-ocular electrode (e.g., positioned in the vitreous or the retina) promotes delivery of the therapeutic agent (e.g., a nucleic acid vector, e.g., a non-viral nucleic acid vector, e.g., a naked nucleic acid vector, e.g., a synthetic circular DNA vector) into a target ocular cell (e.g., retinal cell). Therapeutic agents, e.g., nucleic acid vectors for use in such methods are also provided herein.

In one aspect, the invention provides a method of delivering a therapeutic agent (e.g., a nucleic acid vector, e.g., a non-viral nucleic acid vector, e.g., a naked nucleic acid vector, e.g., synthetic circular DNA vector) into a target retinal cell of an individual, the method comprising: (a) contacting an electrode to an interior region of the eye, wherein an extracellular space in the retina of the eye comprises the therapeutic agent; and (b) while the electrode is contacting the interior region of the eye, transmitting one or more (e.g., 4-12, or 6-10) pulses of electrical energy (e.g., current) through the electrode at conditions suitable for electrotransfer of the therapeutic agent into the target retinal cell. In some embodiments, the electrode is a monopolar electrode (e.g., a monopolar positive electrode positioned in the vitreous, or a monopolar negative electrode positioned in the retina, subretinal space, or a bleb created by subretinal injection of the therapeutic agent). In some embodiments, the electrode is a bipolar electrode (e.g., a bipolar electrode positioned such that the negative electrode is contacting the retina, subretinal space, or a bleb created by the subretinal injection of the therapeutic agent, and the positive electrode is in the vitreous). In other embodiments, the therapeutic agent was delivered to the extracellular space by subretinal injection (e.g., the therapeutic agent has already been administered subretinally and is in position for electrotransfer to the target retinal cells). In other embodiments, the therapeutic agent was delivered to the extracellular space by intravitreal injection. In some embodiments, the delivery of the therapeutic agent to the extracellular space of the retina is also included as part of the aforementioned method. In some embodiments, the delivery of the therapeutic agent (e.g., nucleic acid vector, e.g., non-viral nucleic acid vector, e.g., naked nucleic acid vector, e.g., synthetic circular DNA vector) is by subretinal injection. In other embodiments, the delivery of the therapeutic agent is by intravitreal injection.

In some embodiments in which the therapeutic agent is a nucleic acid vector (e.g., a non-viral nucleic acid vector, e.g., a naked nucleic acid vector, e.g., synthetic circular DNA vector), the nucleic acid is expressed by the target cell (e.g., a retinal cell, e.g., a retinal pigment epithelial (RPE) cell and/or a photoreceptor cell). Thus, methods of delivery described herein can likely be methods of expressing a sequence of interest (e.g., a therapeutic sequence).

In some embodiments, the interior region of the eye contacting the electrode includes the vitreous humor (e.g., the electrode is wholly within the vitreous humor). In some embodiments, the electrode is within 10 mm from the retina upon transmission of the one or more pulses of electrical energy (e.g., within 10 mm, 5 mm, or 1 mm from the retina but not directly contacting the retina). In some embodiments in which the electrode is in the vitreous humor, the electrode is a positive electrode and the voltage applied is a positive voltage (e.g., the electrode is in the vitreous humor, the electrode is a monopolar positive electrode, and the therapeutic agent is a nucleic acid vector (e.g., a DNA vector or an RNA vector), e.g., a non-viral nucleic acid vector, e.g., a naked nucleic acid vector.

In some embodiments, the electrode is directly contacting the retina (and/or the subretinal bleb) upon transmission of the one or more pulses of electrical energy. In some embodiments, the electrode is from 0.1 mm to 10 mm from the retina upon transmission of the one or more pulses of electrical energy. In some embodiments, the electrode is from 0.5 mm to 10 mm from the retina upon transmission of the one or more pulses of electrical energy.

In some embodiments, the interior region of the eye contacting the electrode includes the retina. For example, the electrode may be wholly within the subretinal space, or it may be partially within the subretinal space (e.g., contacting the subretinal bleb). In some embodiments in which the electrode is in contact with the retina, the subretinal space, or the subretinal bleb, the electrode is a negative electrode (e.g., cathode) and the voltage applied is a negative voltage (e.g., the electrode is in contact with the retina, the subretinal space, or the subretinal bleb, the electrode is a monopolar negative electrode (e.g., cathode), and the therapeutic agent is a nucleic acid vector (e.g., any of the DNA vectors or an RNA vectors described herein), e.g., a non-viral nucleic acid vector, e.g., a naked nucleic acid vector.

In some embodiments, the conditions suitable for electrotransfer of the therapeutic agent (e.g., a nucleic acid vector, e.g., a non-viral nucleic acid vector, e.g., a naked nucleic acid vector, e.g., synthetic circular DNA vector) into the target retinal cell comprise a field strength at the target retinal cell from 1 V/cm to 1,500 V/cm (from 1 V/cm to 10 V/cm (e.g., about 1 V/cm, about 2 V/cm, about 3 V/cm, about 4 V/cm, about 5 V/cm, about 6 V/cm, about 7 V/cm, about 8 V/cm, about 9 V/cm, or about 10 V/cm), from about 10 V/cm to about 100 V/cm (e.g., about 10 V/cm, about 20 V/cm, about 30 V/cm, about 40 V/cm, about 50 V/cm, about 60 V/cm, about 70 V/cm, about 80 V/cm, about 90 V/cm, or about 100 V/cm), from about 100 V/cm to about 1,000 V/cm (e.g., about 200 V/cm, about 300 V/cm, about 400 V/cm, about 500 V/cm, about 600 V/cm, about 700 V/cm, about 800 V/cm, about 900 V/cm, or about 1,000 V/cm), or from 1,000 V/cm to 1,500 V/cm (e.g., about 1,000 V/cm, about 1,100 V/cm, about 1,200 V/cm, about 1,300 V/cm, about 1,400 V/cm, or about 1,500 V/cm)). In some embodiments, the field strength at the target cell is from 50 V/cm to 300 V/cm. In some embodiments, the field strength at the target cell is about 100 V/cm.

In some embodiments, the conditions suitable for electrotransfer of the therapeutic agent (e.g., a nucleic acid vector, e.g., a non-viral nucleic acid vector, e.g., a naked nucleic acid vector, e.g., synthetic circular DNA vector) into the target retinal cell comprise a current resulting from the pulsed electric field from 10 μA to 1 A (e.g., from 10 μA to 500 mA, from 10 μA to 200 mA, from 10 μA to 100 mA, from 10 μA to 50 mA, or from 10 μA to 25 mA; e.g., from 50 μA to 500 mA, from 100 μA to 200 mA, or from 1 mA to 100 mA; e.g., from 10 μA to 20 μA, from 20 μA to 30 μA, from 30 μA to 50 μA, from 50 μA to 100 μA, from 100 μA to 150 μA, from 150 μA to 200 μA, from 200 μA to 300 μA, from 300 μA to 400 μA, from 400 μA to 500 μA, from 500 μA to 600 μA, from 600 μA to 800 μA, from 800 μA to 1 mA, from 1 mA to 10 mA, from 10 mA to 20 mA, from 20 mA to 30 mA, from 30 mA to 40 mA, from 40 mA to 50 mA, from 50 mA to 60 mA, from 60 mA to 70 mA, from 70 mA to 80 mA, from 80 mA to 90 mA, from 90 mA to 100 mA, from 100 mA to 200 mA, from 200 mA to 300 mA, from 300 mA to 500 mA, or from 500 mA to 1 A; e.g., about 1 mA, about 5 mA about 10 mA, about 15 mA, about 20 mA, about 25 mA, about 30 mA, about 35 mA, about 40 mA, about 45 mA, about 50 mA, about 60 mA, about 70 mA, about 80 mA, about 90 mA, or about 100 mA).

In some embodiments, 1-3 pulses (e.g., 1 pulse, 2 pulses, or 3 pulses) of energy are transmitted. In some embodiments, 4-12 pulses (e.g., 4, 5, 6, 7, 8, 9, 10, 11, or 12 pulses) of energy are transmitted. In some embodiments, 1-12 pulses are administered. In some embodiments, 10-20 pulses (e.g., 10, 11, 12, 13, 14, 15, 16, 17, 18, 19, or 20 pulses) are administered. In some embodiments, 8 pulses are administered.

In some embodiments, the total number of pulses of electrical energy are delivered within 1-20 seconds. For example, the total number of pulses of electrical energy may be delivered within 1, 2, 3, 4, 5, 6, 7, 8, 9, 10, 11, 12, 13, 14, 15, 16, 17, 18, 19, or 20 seconds. The pulses of electrical energy may be, e.g., square waveforms. The pulses of electrical energy may have an amplitude from 5 V to 1,500 V. For example, the pulses of electrical energy may have an amplitude from about 5 V to 500 V, from about 500 V to about 1,000 V, or from about 1,000 V to about 1,500 V. In some embodiments, the pulses of electrical energy have an amplitude from 5 V to 500 V. For example, the pulses of electrical energy may have an amplitude of about 5V, 10 V, 15 V, 20 V, 25 V, 30 V, 40 V, 50 V, 100 V, 125 V, 150 V, 175 V, 200 V, 225V, 250 V, 275 V, 300 V, 325 V, 350 V, 375 V, 400 V, 425 V, 450 V, 475 V, or 500 V. In some embodiments, the pulses of electrical energy have an amplitude of from about 5 V to about 250 V.

In some embodiments, the pulses of electrical energy have an amplitude of about 20 V. In some embodiments in which the pulses of electrical energy have an amplitude of about 20 V, the current is between 5 mA and 50 mA (e.g., from 10 mA to 40 mA, e.g., from 5 mA to 10 mA, from 10 mA to 15 mA, from 15 mA to 20 mA, from 20 mA to 30 mA, or from 40 mA to 50 mA). In some embodiments, the pulses of electrical energy have an amplitude of about 40 V. In some embodiments in which the pulses of electrical energy have an amplitude of about 40 V, the current is between 10 mA and 100 mA (e.g., from 20 mA to 80 mA, or from 30 mA to 70 mA, e.g., from 10 mA to 20 mA, from 20 mA to 30 mA, from 30 mA to 40 mA, from 40 mA to 50 mA, from 50 mA to 60 mA, from 60 mA to 70 mA, from 70 mA to 80 mA, from 80 mA to 90 mA, or from 90 mA to 100 mA).

In some embodiments, the current resulting from the pulsed electric field is from 10 μA to 1 A (e.g., from 10 μA to 500 mA, from 10 μA to 200 mA, from 10 μA to 100 mA, from 10 μA to 50 mA, or from 10 μA to 25 mA; e.g., from 50 μA to 500 mA, from 100 μA to 200 mA, or from 1 mA to 100 mA; e.g., from 10 μA to 20 μA, from 20 μA to 30 μA, from 30 μA to 50 μA, from 50 μA to 100 μA, from 100 μA to 150 μA, from 150 μA to 200 μA, from 200 μA to 300 μA, from 300 μA to 400 μA, from 400 μA to 500 μA, from 500 μA to 600 μA, from 600 μA to 800 μA, from 800 μA to 1 mA, from 1 mA to 10 mA, from 10 mA to 20 mA, from 20 mA to 30 mA, from 30 mA to 40 mA, from 40 mA to 50 mA, from 50 mA to 60 mA, from 60 mA to 70 mA, from 70 mA to 80 mA, from 80 mA to 90 mA, from 90 mA to 100 mA, from 100 mA to 200 mA, from 200 mA to 300 mA, from 300 mA to 500 mA, or from 500 mA to 1 A; e.g., about 1 mA, about 5 mA about 10 mA, about 15 mA, about 20 mA, about 25 mA, about 30 mA, about 35 mA, about 40 mA, about 45 mA, about 50 mA, about 60 mA, about 70 mA, about 80 mA, about 90 mA, or about 100 mA).

In some embodiments, each of the pulses is from about 0.01 ms to about 200 ms in duration, from about 0.1 ms to about 200 ms in duration, or from about 1 ms to about 200 ms in duration (e.g., 0.10 ms to about 200 ms in duration. For example, each of the pulses may be about 10 ms, 20 ms, 30 ms, 40 ms, 50 ms, 60 ms, 70 ms, 80 ms, 90 ms, 100 ms, 110 ms, 120 ms, 130 ms, 140 ms, 150 ms, 160 ms, 170 ms, 180 ms, 190 ms, or 200 ms). In some embodiments, each of the pulses is about 20 ms in duration. In some embodiments, each of the pulses is about 50 ms in duration. In some embodiments, each of the pulses is from about 0.01 ms to about 1 ms (e.g., from 0.01 ms to 0.05 ms, from 0.05 ms to 0.1 ms, from 0.1 ms to 0.25 ms, from 0.25 ms to 0.5 ms, from 0.5 ms to 0.75 ms, or from 0.75 ms to 1.0 ms; e.g., about 0.01 ms, about 0.05 ms, about 0.1 ms, about 0.2 ms, about 0.3 ms, about 0.4 ms, about 0.5 ms, about 0.6 ms, about 0.7 ms, about 0.8 ms, about 0.9 ms, or about 1.0 ms) in duration.

In some embodiments, the total number of pulses of electrical energy are transmitted within 1-20 seconds (e.g., within 6-12 seconds, e.g., within 1-3 seconds, within 3-6 seconds, within 6-10 seconds, within 10-15 seconds, or within 15-20 seconds, e.g., within one second, within two seconds, within three seconds, within four seconds, within five seconds, within six seconds, within seven seconds, within eight seconds, within nine seconds, within ten seconds, within 11 seconds, within 12 seconds, within 13 seconds, within 14 seconds, within 15 seconds, within 16 seconds, within 17 seconds, within 18 seconds, within 19 seconds, within 20 seconds).

In some embodiments, the pulses of energy are square waveforms. In some embodiments, the pulses of energy have an amplitude from 100 V to 500 V (e.g., from 200 V to 400 V, e.g., from 100 V to 200 V, from 200V to 300 V, from 300V to 400 V, or from 400 V to 500 V, e.g., about 100 V, about 120 V, about 150 V, about 200 V, about 250 V, about 300 V, about 350 V, about 400V, about 450 V, or about 500 V).

In some embodiments, the target retinal cell is a retinal epithelial cell. In some embodiments, the target retinal cell is a photoreceptor. In some embodiments, the target retinal cells are retinal epithelial cells and photoreceptors.

In some embodiments, the therapeutic agent is a nucleic acid vector, e.g., a DNA vector or an RNA vector. In some embodiments, the nucleic acid vector is a non-viral nucleic acid vector (e.g., a non-viral DNA vector or a non-viral RNA vector; e.g., a circular DNA vector or a circular RNA vector). In particular instances, the non-viral nucleic acid vector is a naked nucleic acid vectors (e.g., a naked DNA vector (e.g., a naked circular DNA vector) or a naked RNA vector (e.g., a naked circular RNA vector). In some embodiments in which the DNA vector is a circular DNA vector, the circular DNA vector lacks an origin of replication (e.g., a bacterial original of replication), a drug resistance gene, and/or a recombination site.

In some embodiments, the nucleic acid vector encodes a therapeutic replacement protein that replaces a protein that is endogenously expressed in a healthy retinal cell. In some embodiments, the therapeutic replacement protein replaces a protein that is not endogenously expressed in the target cell of the individual or is non-functional in the target cell of the individual.

In some embodiments, the therapeutic replacement protein is encoded by a coding sequence that is greater than 4.5 kb.

In some embodiments, the therapeutic replacement protein is ABCA4 (e.g., human ABCA4 (e.g., ABCA4 having at least 95% sequence identity with SEQ ID NO: 18, e.g., 100% sequence identity with SEQ ID NO: 18)). In some embodiments, the method is a method of treating an ABCA4-associated retinal dystrophy (e.g., Stargardt Disease).

In some instances of any of the aforementioned embodiments, the nucleic acid vector comprises a nucleic acid sequence that encodes an amino acid sequence having at least 95% sequence identity to SEQ ID NO: 18 (e.g., at least 96% sequence identity to SEQ ID NO: 18, at least 97% sequence identity to SEQ ID NO: 18, at least 98% sequence identity to SEQ ID NO: 18, at least 99% sequence identity to SEQ ID NO: 18, or 100% sequence identity to SEQ ID NO: 18). In some instances, the nucleic acid vector is a naked nucleic acid vector (e.g., a naked DNA vector, (e.g., a naked circular DNA vector (e.g., a plasmid DNA vector, a minicircle DNA vector, or a synthetic circular DNA vector lacking a recombination site (e.g., a supercoiled synthetic circular DNA vector)), a naked closed-ended DNA vector, or a naked doggybone DNA vector) or a naked RNA vector (e.g., a naked circular RNA vector)) comprising a nucleic acid sequence that encodes an amino acid sequence having at least 95% sequence identity to SEQ ID NO: 18 (e.g., at least 96% sequence identity to SEQ ID NO: 18, at least 97% sequence identity to SEQ ID NO: 18, at least 98% sequence identity to SEQ ID NO: 18, at least 99% sequence identity to SEQ ID NO: 18, or 100% sequence identity to SEQ ID NO: 18). In some instances, the nucleic acid vector (e.g., nonviral nucleic acid vector) comprises a nucleic acid sequence that encodes an amino acid sequence having at least 95% sequence identity to SEQ ID NO: 18 (e.g., at least 96% sequence identity to SEQ ID NO: 18, at least 97% sequence identity to SEQ ID NO: 18, at least 98% sequence identity to SEQ ID NO: 18, at least 99% sequence identity to SEQ ID NO: 19, or 100% sequence identity to SEQ ID NO: 18), wherein the nucleic acid vector is encapsulated in a nanoparticle, a microparticle, a liposome, or a lipid nanoparticle. In some instances, such nucleic acid vectors include a CAG promoter.

In some instances, the nucleic acid vector comprises or consists of a nucleic acid sequence having at least 95% sequence identity to SEQ ID NO: 19 (e.g., at least 96% sequence identity to SEQ ID NO: 19, at least 97% sequence identity to SEQ ID NO: 19, at least 98% sequence identity to SEQ ID NO: 19, at least 99% sequence identity to SEQ ID NO: 19, or 100% sequence identity to SEQ ID NO: 19). In some instances, the nucleic acid vector comprises or consists of the nucleic acid sequence of SEQ ID NO: 19. In some instances, the nucleic acid vector is a naked nucleic acid vector (e.g., a naked DNA vector, (e.g., a naked circular DNA vector (e.g., a plasmid DNA vector, a minicircle DNA vector, or a synthetic circular DNA vector lacking a recombination site (e.g., a supercoiled synthetic circular DNA vector)), a naked closed-ended DNA vector, or a naked doggybone DNA vector) or a naked RNA vector (e.g., a naked circular RNA vector)) comprising or consisting of a nucleic acid sequence having at least 95% sequence identity to SEQ ID NO: 19 (e.g., at least 96% sequence identity to SEQ ID NO: 19, at least 97% sequence identity to SEQ ID NO: 19, at least 98% sequence identity to SEQ ID NO: 19, at least 99% sequence identity to SEQ ID NO: 19, or 100% sequence identity to SEQ ID NO: 19). In some instances, the nucleic acid vector (e.g., nonviral nucleic acid vector) comprises or consists of a nucleic acid sequence having at least 95% sequence identity to SEQ ID NO: 19 (e.g., at least 96% sequence identity to SEQ ID NO: 19, at least 97% sequence identity to SEQ ID NO: 19, at least 98% sequence identity to SEQ ID NO: 19, at least 99% sequence identity to SEQ ID NO: 19, or 100% sequence identity to SEQ ID NO: 19), wherein the nucleic acid vector is encapsulated in a nanoparticle, a microparticle, a liposome, or a lipid nanoparticle.

In some embodiments, the therapeutic replacement protein is encoded by a coding sequence that is greater than 4.5 kb. In some embodiments, the therapeutic replacement protein is MYO7A. In some embodiments, the method is a method of treating Usher syndrome 1B in the individual.

In some embodiments, the therapeutic replacement protein is BEST1. In some embodiments, the method is a method of treating a bestrophinopathy associated with a BEST1 dominant mutation or a BEST1 recessive mutation.

In some embodiments, the therapeutic replacement protein is CFH. In some embodiments, the method is a method of treating age-related macular degeneration.

In another aspect, provided herein is a nucleic acid vector (or a pharmaceutical composition thereof) comprising a nucleic acid sequence driven by a CAG promoter that encodes an amino acid sequence having at least 95% sequence identity to SEQ ID NO: 18 (e.g., at least 96% sequence identity to SEQ ID NO: 18, at least 97% sequence identity to SEQ ID NO: 18, at least 98% sequence identity to SEQ ID NO: 18, at least 99% sequence identity to SEQ ID NO: 18, or 100% sequence identity to SEQ ID NO: 18). In some instances, the nucleic acid vector is a naked nucleic acid vector (e.g., a naked DNA vector, (e.g., a naked circular DNA vector (e.g., a plasmid DNA vector, a minicircle DNA vector, or a synthetic circular DNA vector lacking a recombination site (e.g., a supercoiled synthetic circular DNA vector)), a naked closed-ended DNA vector, or a naked doggybone DNA vector) or a naked RNA vector (e.g., a naked circular RNA vector)) comprising a nucleic acid sequence that encodes an amino acid sequence having at least 95% sequence identity to SEQ ID NO: 18 (e.g., at least 96% sequence identity to SEQ ID NO: 18, at least 97% sequence identity to SEQ ID NO: 18, at least 98% sequence identity to SEQ ID NO: 18, at least 99% sequence identity to SEQ ID NO: 18, or 100% sequence identity to SEQ ID NO: 18). In some instances, the nucleic acid vector (e.g., nonviral nucleic acid vector) comprises a nucleic acid sequence that encodes an amino acid sequence having at least 95% sequence identity to SEQ ID NO: 18 (e.g., at least 96% sequence identity to SEQ ID NO: 18, at least 97% sequence identity to SEQ ID NO: 18, at least 98% sequence identity to SEQ ID NO: 18, at least 99% sequence identity to SEQ ID NO: 19, or 100% sequence identity to SEQ ID NO: 18), wherein the nucleic acid vector is encapsulated in a nanoparticle, a microparticle, a liposome, or a lipid nanoparticle.

In another aspect, the invention provides a nucleic acid vector (or pharmaceutical composition thereof) comprising or consisting of a nucleic acid sequence having at least 95% sequence identity to SEQ ID NO: 19 (e.g., at least 96% sequence identity to SEQ ID NO: 19, at least 97% sequence identity to SEQ ID NO: 19, at least 98% sequence identity to SEQ ID NO: 19, at least 99% sequence identity to SEQ ID NO: 19, or 100% sequence identity to SEQ ID NO: 19). In some instances, the nucleic acid vector comprises or consists of the nucleic acid sequence of SEQ ID NO: 19. In some instances, the nucleic acid vector is a naked nucleic acid vector (e.g., a naked DNA vector, (e.g., a naked circular DNA vector (e.g., a plasmid DNA vector, a minicircle DNA vector, or a synthetic circular DNA vector lacking a recombination site (e.g., a supercoiled synthetic circular DNA vector)), a naked closed-ended DNA vector, or a naked doggybone DNA vector) or a naked RNA vector (e.g., a naked circular RNA vector)) comprising or consisting of a nucleic acid sequence having at least 95% sequence identity to SEQ ID NO: 19 (e.g., at least 96% sequence identity to SEQ ID NO: 19, at least 97% sequence identity to SEQ ID NO: 19, at least 98% sequence identity to SEQ ID NO: 19, at least 99% sequence identity to SEQ ID NO: 19, or 100% sequence identity to SEQ ID NO: 19). In some instances, the nucleic acid vector (e.g., nonviral nucleic acid vector) comprises or consists of a nucleic acid sequence having at least 95% sequence identity to SEQ ID NO: 19 (e.g., at least 96% sequence identity to SEQ ID NO: 19, at least 97% sequence identity to SEQ ID NO: 19, at least 98% sequence identity to SEQ ID NO: 19, at least 99% sequence identity to SEQ ID NO: 19, or 100% sequence identity to SEQ ID NO: 19), wherein the nucleic acid vector is encapsulated in a nanoparticle, a microparticle, a liposome, or a lipid nanoparticle.

In some embodiments, the therapeutic sequence or therapeutic protein (e.g., therapeutic replacement protein) is shown in Table 1.

In another aspect, the invention provides a method of delivering a non-viral (e.g., naked) synthetic circular DNA vector encoding a retinal protein (e.g., ABCA4, MYO7A, or CEP290) into a target retinal cell of an individual (e.g., a human), the method comprising: (a) contacting a monopolar needle electrode (e.g., negative electrode, e.g., cathode) to a retina or subretinal bleb in an individual, wherein an extracellular space in the retina comprises the synthetic circular DNA vector; and (b) while the electrode is contacting the retina or the subretinal bleb, applying six-to-ten (e.g., eight) 20-40V pulses to the electrode, each having a duration from 10-30 ms (e.g., about 20 ms) over the course of 1 second to 30 seconds, e.g., about 8 seconds. In some embodiments, the non-viral (e.g., naked) synthetic circular DNA vector was delivered to the extracellular space in the retina by subretinal injection. In some embodiments, the delivery of the non-viral (e.g., naked) synthetic circular DNA vector to the extracellular space of the retina is also included as part of the aforementioned method. In some embodiments, the nucleic acid is expressed by the target cell (e.g., a retinal cell, e.g., an RPE cell and/or a photoreceptor cell). In some embodiments, the method treats or prevents an ocular disorder associated with the retinal protein expressed by the treatment.

In another aspect, the invention provides a method of delivering a non-viral (e.g., naked) synthetic circular DNA vector encoding a retinal protein (e.g., ABCA4, MYO7A, or CEP290) into a target retinal cell of an individual (e.g., a human), the method comprising: (a) contacting a monopolar needle electrode (e.g., a monopolar positive needle electrode, e.g., anode) to a vitreous humor in an individual, such that the distal end of the electrode is within 1 mm of the retina, wherein an extracellular space in the retina comprises the synthetic circular DNA vector; and (b) while the electrode is contacting the vitreous humor within 1 mm of the retina, applying six-to-ten (e.g., eight) 20-40V pulses to the electrode, each having a duration from 10-30 ms (e.g., about 20 ms) over the course of 1 second to 30 seconds, e.g., about 8 seconds. In some embodiments, the non-viral (e.g., naked) synthetic circular DNA vector was delivered to the extracellular space in the retina by subretinal injection. In some embodiments, the delivery of the non-viral (e.g., naked) synthetic circular DNA vector to the extracellular space of the retina is also included as part of the aforementioned method. In some embodiments, the nucleic acid is expressed by the target cell (e.g., a retinal cell, e.g., an RPE cell and/or a photoreceptor cell). In some embodiments, the method treats or prevents an ocular disorder associated with the retinal protein expressed by the treatment. The present invention also provides approaches for delivering or expressing therapeutic agents (e.g., nucleic acid vectors encoding therapeutic replacement proteins) to ocular cells (e.g., retinal cells) by suprachoroidal administration. In some instances, approaches described herein involve electrotransfer, a process in which transmission of an electric field into an ocular tissue (e.g., retina) promotes delivery of the therapeutic agent into a target ocular cell (e.g., retinal cell).

In another aspect, the invention provides a method of delivering a therapeutic agent (e.g., a nucleic acid vector, e.g., a non-viral nucleic acid vector, e.g., a naked nucleic acid vector, e.g., synthetic circular DNA vector) into a target retinal cell of an individual, the method comprising: (a) contacting an electrode to an interior region of the eye, wherein an extracellular space in the retina of the eye comprises a therapeutic agent delivered by suprachoroidal injection; and (b) while the electrode is contacting the interior region of the eye, transmitting one or more pulses of electrical energy (e.g., current) through the electrode at conditions suitable for electrotransfer of the therapeutic agent into the target retinal cell. In some embodiments, the electrode is a monopolar electrode. In some embodiments, the electrode is a bipolar electrode.

In some embodiments, the delivery of the therapeutic agent (e.g., a nucleic acid vector, e.g., a non-viral nucleic acid vector, e.g., a naked nucleic acid vector, e.g., synthetic circular DNA vector) to the extracellular space of the retina is also included as part of the aforementioned method. In some embodiments, the delivery of the therapeutic agent is by suprachoroidal injection (e.g., bilateral suprachoroidal injection). In some embodiments, the electrotransfer is administered after delivery of the therapeutic agent. In some embodiments, the electrotransfer is administered before delivery of the therapeutic agent.

In some embodiments, the interior region of the eye contacting the electrode includes the vitreous humor (e.g., the electrode is wholly within the vitreous humor). In some embodiments, the electrode is within 10 mm from the retina upon transmission of the one or more pulses of electrical energy (e.g., within 10 mm from the retinal but not directly contacting the retina). In some embodiments, the electrode is directly contacting the retina upon transmission of the one or more pulses of electrical energy. In some embodiments, the electrode is from 0.1 mm to 10 mm from the retina upon transmission of the one or more pulses of electrical energy. In some embodiments, the electrode is from 0.5 mm to 10 mm from the retina upon transmission of the one or more pulses of electrical energy.

In some embodiments, the interior region of the eye contacting the electrode includes the retina. For example, the electrode may be wholly within the subretinal space, or it may be partially within the subretinal space.

In some embodiments, the conditions suitable for electrotransfer of the therapeutic agent (e.g., a nucleic acid vector, e.g., a non-viral nucleic acid vector, e.g., a naked nucleic acid vector, e.g., synthetic circular DNA vector) into the target retinal cell comprise a field strength at the target retinal cell from 1 V/cm to 1,500 V/cm (from 1 V/cm to 10 V/cm (e.g., about 1 V/cm, about 2 V/cm, about 3 V/cm, about 4 V/cm, about 5 V/cm, about 6 V/cm, about 7 V/cm, about 8 V/cm, about 9 V/cm, or about 10 V/cm), from about 10 V/cm to about 100 V/cm (e.g., about 10 V/cm, about 20 V/cm, about 30 V/cm, about 40 V/cm, about 50 V/cm, about 60 V/cm, about 70 V/cm, about 80 V/cm, about 90 V/cm, or about 100 V/cm), from about 100 V/cm to about 1,000 V/cm (e.g., about 200 V/cm, about 300 V/cm, about 400 V/cm, about 500 V/cm, about 600 V/cm, about 700 V/cm, about 800 V/cm, about 900 V/cm, or about 1,000 V/cm), or from 1,000 V/cm to 1,500 V/cm (e.g., about 1,000 V/cm, about 1,100 V/cm, about 1,200 V/cm, about 1,300 V/cm, about 1,400 V/cm, or about 1,500 V/cm)). In some embodiments, the field strength at the target cell is from 50 V/cm to 300 V/cm. In some embodiments, the field strength at the target cell is about 100 V/cm.

In some embodiments, 1-3 pulses (e.g., 1 pulse, 2 pulses, or 3 pulses) of energy are transmitted. In some embodiments, 4-12 pulses (e.g., 4, 5, 6, 7, 8, 9, 10, 11, or 12 pulses) of energy are transmitted. In some embodiments, 1-12 pulses are administered. In some embodiments, 10-20 pulses (e.g., 10, 11, 12, 13, 14, 15, 16, 17, 18, 19, or 20 pulses) are administered.

In some embodiments, the total number of pulses of electrical energy are delivered within 1-20 seconds. For example, the total number of pulses of electrical energy may be delivered within 1, 2, 3, 4, 5, 6, 7, 8, 9, 10, 11, 12, 13, 14, 15, 16, 17, 18, 19, or 20 seconds. The pulses of electrical energy may be, e.g., square waveforms. The pulses of electrical energy may have an amplitude from 5 V to 1,500 V. For example, the pulses of electrical energy may have an amplitude from about 5 V to 500 V, from about 500 V to about 1,000 V, or from about 1,000 V to about 1,500 V. In some embodiments, the pulses of electrical energy have an amplitude from 5 V to 500 V. For example, the pulses of electrical energy may have an amplitude of about 100 V, 125 V, 150 V, 175 V, 200 V, 225V, 250 V, 275 V, 300 V, 325 V, 350 V, 375 V, 400 V, 425 V, 450 V, 475 V, or 500 V. In some embodiments, the pulses of electrical energy have an amplitude of from about 5 V to about 250 V. In some embodiments, each of the pulses is from about 0.01 ms to about 200 ms in duration, from about 0.1 ms to about 200 ms in duration, or from about 1 ms to about 200 ms in duration (e.g., 0.10 ms to about 200 ms in duration. For example, each of the pulses may be about 10 ms, 20 ms, 30 ms, 40 ms, 50 ms, 60 ms, 70 ms, 80 ms, 90 ms, 100 ms, 110 ms, 120 ms, 130 ms, 140 ms, 150 ms, 160 ms, 170 ms, 180 ms, 190 ms, or 200 ms). In some embodiments, each of the pulses is about 20 ms in duration. In some embodiments, each of the pulses is about 50 ms in duration. In some embodiments, each of the pulses is from about 0.01 ms to about 1 ms (e.g., from 0.01 ms to 0.05 ms, from 0.05 ms to 0.1 ms, from 0.1 ms to 0.25 ms, from 0.25 ms to 0.5 ms, from 0.5 ms to 0.75 ms, or from 0.75 ms to 1.0 ms; e.g., about 0.01 ms, about 0.05 ms, about 0.1 ms, about 0.2 ms, about 0.3 ms, about 0.4 ms, about 0.5 ms, about 0.6 ms, about 0.7 ms, about 0.8 ms, about 0.9 ms, or about 1.0 ms) in duration.

In some embodiments, the total number of pulses of electrical energy are transmitted within 1-20 seconds (e.g., within 6-12 seconds, e.g., within 1-3 seconds, within 3-6 seconds, within 6-10 seconds, within 10-15 seconds, or within 15-20 seconds, e.g., within one second, within two seconds, within three seconds, within four seconds, within five seconds, within six seconds, within seven seconds, within eight seconds, within nine seconds, within ten seconds, within 11 seconds, within 12 seconds, within 13 seconds, within 14 seconds, within 15 seconds, within 16 seconds, within 17 seconds, within 18 seconds, within 19 seconds, within 20 seconds).

In some embodiments, the pulses of energy are square waveforms. In some embodiments, the pulses of energy have an amplitude from 100 V to 500 V (e.g., from 200 V to 400 V, e.g., from 100 V to 200 V, from 200V to 300 V, from 300V to 400 V, or from 400 V to 500 V, e.g., about 100 V, about 120 V, about 150 V, about 200 V, about 250 V, about 300 V, about 350 V, about 400V, about 450 V, or about 500 V).

In some embodiments, the target retinal cell is a retinal epithelial cell. In some embodiments, the target retinal cell is a photoreceptor. In some embodiments, the target retinal cells are retinal epithelial cells and photoreceptors.

In some embodiments, the therapeutic agent is a nucleic acid vector, e.g., a DNA vector or an RNA vector. In some embodiments, the nucleic acid vector is a non-viral nucleic acid vector (e.g., a non-viral DNA vector or a non-viral RNA vector; e.g., a circular DNA vector or a circular RNA vector). In particular instances, the non-viral nucleic acid vector is a naked nucleic acid vectors (e.g., a naked DNA vector (e.g., a naked circular DNA vector) or a naked RNA vector (e.g., a naked circular RNA vector). In some embodiments in which the DNA vector is a circular DNA vector, the circular DNA vector lacks an origin of replication (e.g., a bacterial original of replication), a drug resistance gene, and/or a recombination site.

In some embodiments, the nucleic acid vector encodes a therapeutic replacement protein that replaces a protein that is endogenously expressed in a healthy retinal cell. In some embodiments, the therapeutic replacement protein replaces a protein that is not endogenously expressed in the target cell of the individual or is non-functional in the target cell of the individual.

In some embodiments, the therapeutic replacement protein is encoded by a coding sequence that is greater than 4.5 kb. In some embodiments, the therapeutic replacement protein is MYO7A. In some embodiments, the method is a method of treating Ushers syndrome 1B in the individual.

In some embodiments, the therapeutic replacement protein is BEST1. In some embodiments, the method is a method of treating a bestrophinopathy associated with a BEST1 dominant mutation or a BEST1 recessive mutation.

In some embodiments, the therapeutic replacement protein is CFH. In some embodiments, the method is a method of treating age-related macular degeneration.

In another aspect, the invention provides a method of treating a retinal dystrophy comprising suprachoroidally injecting a circular DNA vector (e.g., a naked circular DNA vector) into the eye of an individual having a retinal dystrophy, wherein the retinal dystrophy is characterized by a lack of expression of a retinal protein. In some embodiments, the circular DNA vector comprises one or more therapeutic genes encoding a therapeutic replacement protein to replace the retinal protein. In some embodiments, the circular DNA vector lacks a bacterial origin or replication and/or a drug resistance gene (e.g., the circular DNA vector lacks a bacterial origin or replication, a drug resistance gene, and a recombination site). In some embodiments, the method further comprises: (a) contacting an electrode to an interior region of the eye; and (b) while the electrode is contacting the interior region of the eye, transmitting one or more pulses of electrical energy (e.g., current) through the electrode at conditions suitable for electrotransfer of the circular DNA vector into a target retinal cell. In some embodiments, the electrode is a monopolar electrode. In some embodiments, the electrode is a bipolar electrode.

In some embodiments, the interior region of the eye contacting the electrode includes the vitreous humor (e.g., the electrode is wholly within the vitreous humor). In some embodiments, the electrode is from 0.1 mm to 10 mm from the retina upon transmission of the one or more pulses of electrical energy.

In some embodiments, the interior region of the eye contacting the electrode includes the retina. For example, the electrode may be wholly within the subretinal space, or it may be partially within the subretinal space.

In some embodiments, the conditions suitable for electrotransfer of the therapeutic agent into the target retinal cell comprise a field strength at the target retinal cell from 1 V/cm to 1,500 V/cm (from 1 V/cm to 10 V/cm (e.g., about 1 V/cm, about 2 V/cm, about 3 V/cm, about 4 V/cm, about 5 V/cm, about 6 V/cm, about 7 V/cm, about 8 V/cm, about 9 V/cm, or about 10 V/cm,), from about 10 V/cm to about 100 V/cm (e.g., about 10 V/cm, about 20 V/cm, about 30 V/cm, about 40 V/cm, about 50 V/cm, about 60 V/cm, about 70 V/cm, about 80 V/cm, about 90 V/cm, or about 100 V/cm), from about 100 V/cm to about 1,000 V/cm (e.g., about 200 V/cm, about 300 V/cm, about 400 V/cm, about 500 V/cm, about 600 V/cm, about 700 V/cm, about 800 V/cm, about 900 V/cm, or about 1,000 V/cm), or from 1,000 V/cm to 1,500 V/cm (e.g., about 1,000 V/cm, about 1,100 V/cm, about 1,200 V/cm, about 1,300 V/cm, about 1,400 V/cm, or about 1,500 V/cm)). In some embodiments, the field strength at the target cell is from 50 V/cm to 300 V/cm. In some embodiments, the field strength at the target cell is about 100 V/cm.

In some embodiments, 4-12 pulses (e.g., 4, 5, 6, 7, 8, 9, 10, 11, or 12 pulses) of energy are transmitted. In some embodiments, 10-20 pulses (e.g., 10, 11, 12, 13, 14, 15, 16, 17, 18, 19, or 20 pulses) are administered.

In some embodiments, the total number of pulses of electrical energy are delivered within 1-20 seconds. For example, the total number of pulses of electrical energy may be delivered within 1, 2, 3, 4, 5, 6, 7, 8, 9, 10, 11, 12, 13, 14, 15, 16, 17, 18, 19, or 20 seconds. The pulses of electrical energy may be, e.g., square waveforms. The pulses of electrical energy may have an amplitude from 5 V to 1,500 V. For example, the pulses of electrical energy may have an amplitude from about 5 V to 500 V, from about 500 V to about 1,000 V, or from about 1,000 V to about 1,500 V. In some embodiments, the pulses of electrical energy have an amplitude from 5 V to 500 V. For example, the pulses of electrical energy may have an amplitude of about 100 V, 125 V, 150 V, 175 V, 200 V, 225V, 250 V, 275 V, 300 V, 325 V, 350 V, 375 V, 400 V, 425 V, 450 V, 475 V, or 500 V. In some embodiments, the pulses of electrical energy have an amplitude of from about 5 V to about 250 V. In some embodiments, each of the pulses is from about 0.01 ms to about 200 ms in duration, from about 0.1 ms to about 200 ms in duration, or from about 1 ms to about 200 ms in duration (e.g., 0.10 ms to about 200 ms in duration. For example, each of the pulses may be about 10 ms, 20 ms, 30 ms, 40 ms, 50 ms, 60 ms, 70 ms, 80 ms, 90 ms, 100 ms, 110 ms, 120 ms, 130 ms, 140 ms, 150 ms, 160 ms, 170 ms, 180 ms, 190 ms, or 200 ms). In some embodiments, each of the pulses is about 20 ms in duration. In some embodiments, each of the pulses is about 50 ms in duration.

In some embodiments, the total number of pulses of electrical energy are transmitted within 1-20 seconds (e.g., within 6-12 seconds, e.g., within 1-3 seconds, within 3-6 seconds, within 6-10 seconds, within 10-15 seconds, or within 15-20 seconds, e.g., within one second, within two seconds, within three seconds, within four seconds, within five seconds, within six seconds, within seven seconds, within eight seconds, within nine seconds, within ten seconds, within 11 seconds, within 12 seconds, within 13 seconds, within 14 seconds, within 15 seconds, within 16 seconds, within 17 seconds, within 18 seconds, within 19 seconds, within 20 seconds).

In some embodiments, the pulses of energy are square waveforms. In some embodiments, the pulses of energy have an amplitude from 100 V to 500 V (e.g., from 200 V to 400 V, e.g., from 100 V to 200 V, from 200V to 300 V, from 300V to 400 V, or from 400 V to 500 V, e.g., about 100 V, about 120 V, about 150 V, about 200 V, about 250 V, about 300 V, about 350 V, about 400V, about 450 V, or about 500 V).

In some embodiments, the target retinal cell is a retinal epithelial cell. In some embodiments, the target retinal cell is a photoreceptor. In some embodiments, the target retinal cells are retinal epithelial cells and photoreceptors.

In some embodiments, the therapeutic agent is a nucleic acid vector, e.g., a DNA vector or an RNA vector. In some embodiments, the nucleic acid vector is a non-viral nucleic acid vector (e.g., a non-viral DNA vector or a non-viral RNA vector; e.g., a circular DNA vector or a circular RNA vector). In particular instances, the non-viral nucleic acid vector is a naked nucleic acid vectors (e.g., a naked DNA vector (e.g., a naked circular DNA vector) or a naked RNA vector (e.g., a naked circular RNA vector). In some embodiments in which the DNA vector is a circular DNA vector, the circular DNA vector lacks an origin of replication (e.g., a bacterial original of replication), a drug resistance gene, and/or a recombination site.

In some embodiments, the nucleic acid vector encodes a therapeutic replacement protein that replaces a protein that is endogenously expressed in a healthy retinal cell. In some embodiments, the therapeutic replacement protein replaces a protein that is not endogenously expressed in the target cell of the individual or is non-functional in the target cell of the individual.

In some embodiments, the therapeutic replacement protein is encoded by a coding sequence that is greater than 4.5 kb. In some embodiments, the therapeutic replacement protein is MYO7A. In some embodiments, the method is a method of treating Ushers syndrome 1B in the individual.

In some embodiments, the therapeutic replacement protein is BEST1. In some embodiments, the method is a method of treating a bestrophinopathy associated with a BEST1 dominant mutation or a BEST1 recessive mutation.

In some embodiments, the therapeutic replacement protein is CFH. In some embodiments, the method is a method of treating age-related macular degeneration.

In some embodiments, the therapeutic replacement protein is shown in Table 1.

The present invention also provides devices and methods to deliver therapeutic agents (e.g., nucleic acid vectors) to target cells via electrotransfer. Such devices and methods, in general, employ transmission of an electric field by the device into a tissue, which promotes delivery of the therapeutic agent into a target cell within that tissue. The present devices are designed to transmit an electric field shaped to match an internal topography of a target tissue interface (e.g., a substantially planar, curved, or spherical topography), thereby increasing the number of target cells exposed to an effective electric field and, in turn, improving efficiency of electrotransfer of the therapeutic agent. In particular uses of such devices, retinal cells can be transfected with nucleic acid vectors with high efficiency.

In one aspect, a device includes a sheath having a proximal end, a distal end, and a longitudinal axis therebetween. The device further includes an elongate conductor having a proximal portion within the sheath and a distal portion, wherein the elongate conductor includes a preformed shape memory material and is retractable within the sheath from a proximal position to a distal position. In the proximal position, the distal portion of the elongate conductor is substantially straight. In the distal position, the distal portion of the elongate conductor extends beyond the distal end of the sheath, and the shape memory material of the distal portion of the elongate conductor is relaxed radially to form an electrode that is disposed at a preformed angle (e.g., from about 10° to about 170°, e.g., from about 20° to about 160°, e.g., from about 30° to about 150°, e.g., from about 45° to about 135°, e.g., from about 60° to about 120°, e.g., from about 70° to about 110°, e.g., from about 80° to about 100°, e.g., from about 85° to about 95°, e.g., about 10°, 20°,30°, 45°, 50°, 55°, 60°, 65°, 70°, 71°, 72°, 73°, 74°, 75°, 76°, 77°, 78°, 79°, 80°, 81°, 82°, 83°, 84°, 85°, 86°, 87°, 88°, 89°, 90°, 91°, 92°, 93°, 94°, 95°, 96°, 97°, 98°, 99°, 100°, 101°, 102°, 103°, 104°, 105°, 106°, 107°, 108°, 109°, 110°, 115°, 120°, 125°, 130°, 135°, 140°, 145°, 150°, 160°, or 170°) relative to the longitudinal axis of the sheath. In some embodiments, the electrode is a substantially planar electrode. In some embodiments, the preformed angle is substantially a right angle. In some embodiments, the preformed angle is about 70 degrees or about 110 degrees.

In another aspect, a device includes a sheath having a proximal end, a distal end, and a longitudinal axis therebetween. The device further includes an elongate conductor having a proximal portion within the sheath and a distal portion, wherein the elongate conductor includes a preformed shape memory material and is retractable within the sheath from a proximal position to a distal position. In the proximal position, the distal portion of the elongate conductor is substantially straight. In the distal position, the distal portion of the elongate conductor extends beyond the distal end of the sheath, and the shape memory material of the distal portion of the elongate conductor is relaxed radially to form an electrode that is approximately perpendicular to the longitudinal axis of the sheath. In some embodiments, the electrode is a substantially planar electrode.

In another aspect, a device includes a sheath having a proximal end, a distal end, and a longitudinal axis therebetween. The device further includes an elongate conductor having a proximal portion within the sheath and a distal portion, wherein the elongate conductor includes a preformed shape memory material and is retractable within the sheath from a proximal position to a distal position. In the proximal position, the distal portion of the elongate conductor is substantially straight. In the distal position, the distal portion of the elongate conductor extends beyond the distal end of the sheath, and the shape memory material of the distal portion of the elongate conductor is relaxed radially to form an electrode that is at substantially a right angle to the longitudinal axis of the sheath. In some embodiments, the electrode is a substantially planar electrode. In some embodiments, the substantially right angle is about 70 degrees or about 110 degrees.

In some embodiments, the device further includes a handle having a proximal end and a distal end. The sheath may be connected (e.g., immobilized) to the handle.

In some embodiments, the proximal end of the sheath is connected to (e.g., disposed within) the handle.

In some embodiments, a distal portion of the handle includes a hollow region between an inner surface of the handle and the elongate conductor therewithin, and the proximal end of the sheath is disposed within the hollow region within the handle.

In some embodiments, the proximal end of the sheath is disposed at least 1 mm, 2 mm, 3 mm, 4 mm, 5 mm, 6 mm, 7 mm, 8 mm, 9 mm, 10 mm, 15 mm, 20 mm, 25 mm, 30 mm, 35 mm, 40 mm, 45 mm, 50 mm, 55 mm, 60 mm, 65 mm, 70 mm, 80 mm, 85 mm, 90 mm, 95 mm, 100 mm, 110 mm, 120 mm, 130 mm, 140 mm, 150 mm, 160 mm, 170 mm, 180 mm, 190 mm, 200 mm, or more within the hollow region.

In some embodiments, the handle is cylindrical.

In some embodiments, the handle further includes a cap on the distal and/or proximal end of the handle.

In some embodiments, the device further includes an actuator that is configured to slide the elongate conductor between the proximal position and the distal position.

In some embodiments, the proximal end of the sheath and/or the elongate conductor is connected to the actuator. The actuator may be configured to slide the elongate conductor between the proximal position and the distal position. In some embodiments, actuator is a slider. The slider has a proximal end and a distal end and is attached to the elongate conductor. The slider is configured to retract the elongate conductor from the distal position to the proximal position along the longitudinal axis of the sheath.

In some embodiments, the slider includes a proximal position and a distal position. In the proximal position, the proximal end of the sheath may be disposed at or proximal to the distal end of the slider. In the distal position, the proximal end of the sheath may be disposed between the proximal end of the slider and the distal end of the slider.

In some embodiments, the slider is configured to stop upon sliding to the distal position and/or the proximal position.

In some embodiments, the slider is disposed in the distal position and the distal portion of the elongate conductor extends beyond the distal end of the sheath. The shape memory material of the distal portion of the elongate conductor may be relaxed radially to form a substantially planar electrode at the preformed angle relative to the longitudinal axis of the sheath.

In some embodiments, the slider is disposed in the proximal position and the distal portion of the elongate conductor is substantially straight.

In some embodiments, the slider includes a control member disposed on an exterior of the handle. The control member and the slider may be integral. Alternatively, the control member and the slider may be non-integral.

In another aspect, a device includes a handle having a proximal end and a distal end. The device further includes a sheath having a proximal end, a distal end, and a longitudinal axis therebetween. The sheath may be connected (e.g., immobilized) to the handle. The proximal end of the sheath may be connected to (e.g., disposed within) the handle. The device also includes an elongate conductor having a proximal portion within the sheath and a distal portion, and the elongate conductor includes a preformed shape memory material and is retractable within the sheath from a proximal position to a distal position. In the proximal position, the distal portion of the elongate conductor is substantially straight. In the distal position, the distal portion of the elongate conductor extends beyond the distal end of the sheath, and the shape memory material of the distal portion of the elongate conductor is relaxed radially to form a substantially planar electrode disposed at a preformed angle (e.g., from about 10′ to about 170°, e.g., from about 20° to about 160°, e.g., from about 30° to about 150°, e.g., from about 45° to about 135°, e.g., from about 60° to about 120°, e.g., from about 70° to about 110°, e.g., from about 80° to about 100°, e.g., from about 85° to about 95°, e.g., about 10°, 20°,30°, 45°, 50°, 55°, 60°, 65°, 70°, 71°, 72°, 73°, 74°, 75°, 76°, 77°, 78°, 79°, 80°, 81°, 82°, 83°, 84°, 85°, 86°, 87°, 88°, 89°, 90°, 91°, 92°, 93°, 94°, 95°, 96°, 97°, 98°, 99°, 100°, 101°, 102°, 103°, 104°, 105°, 106°, 107°, 108°, 109°, 110°, 115°, 120°, 125°, 130°, 135°, 140°, 145°, 150°, 160°, or 170°) relative to the longitudinal axis of the sheath. The device also includes a slider having a proximal end and a distal end and attached to the elongate conductor. The slider is configured to retract the elongate conductor from the distal position to the proximal position along the longitudinal axis of the sheath. In some embodiments, the preformed angle is about 70 degrees or about 110 degrees.

In some embodiments, the device further includes a sheath connected (e.g., immobilized) to the slider. The elongate conductor may be within the sheath connected to the slider. In some embodiments, the sheath connected to the slider nests with the sheath connected (e.g., immobilized) to the handle. The sheath connected to the slider may be configured to be surrounded by the sheath connected to the handle or a portion thereof. For example, the sheath connected to the slider may have a diameter that is less than the diameter of the sheath connected to the handle. Alternatively, the sheath connected to the slider may surround the sheath connected to the handle or a portion thereof. For example, the sheath connected to the slider may have a diameter that is greater than the diameter of the sheath connected to the handle. In some embodiments, the sheath connected to the slider is connected to the elongate conductor, e.g., at the proximal end of the elongate conductor.

In some embodiments, the distal end of the sheath includes a needle (e.g., a hypodermic needle).

In some embodiments, the device further includes an insulator, e.g., between the proximal portion of the elongate conductor and the sheath.

In some embodiments, the sheath includes a conductive material.

The inner diameter of the sheath may be from about 0.01 mm to about 5 mm, e.g., from about 0.1 mm to about 1 mm, e.g., from about 0.2 mm to about 0.5 mm, e.g., from about 0.2 mm to about 0.3 mm. For example, the inner diameter of the sheath may be from 0.01 mm to about 5 mm, e.g., from about 0.01 mm to about 0.1 mm, e.g., about 0.02 mm, 0.03 mm, 0.04 mm, 0.05 mm, 0.06 mm, 0.07 mm, 0.08 mm, 0.09 mm, or 0.1 mm, e.g., from about 0.1 mm to about 1 mm, e.g., about 0.2 mm, 0.3 mm, 0.4 mm, 0.5 mm, 0.6 mm, 0.7 mm, 0.8 mm, 0.9 mm, or 1 mm, e.g., from about 1 mm to about 5 mm, e.g., about 2 mm, 2.5 mm, 3 mm, 3.5 mm, 4 mm, 4.5 mmm, or 5 mm. In some embodiments, the sheath has an inner diameter of about 0.1 mm to about 1 mm. In some embodiments, the sheath has an inner diameter of about 0.2 mm to about 0.3 mm.

The outer diameter of the sheath, which is greater than the inner diameter, may be from about 0.01 mm to about 5 mm, e.g., from about 0.1 mm to about 1 mm, e.g., from about 0.2 mm to about 0.5 mm, e.g., from about 0.2 mm to about 0.3 mm. For example, the inner diameter of the sheath may be from 0.01 mm to about 5 mm, e.g., from about 0.01 mm to about 0.1 mm, e.g., about 0.02 mm, 0.03 mm, 0.04 mm, 0.05 mm, 0.06 mm, 0.07 mm, 0.08 mm, 0.09 mm, or 0.1 mm, e.g., from about 0.1 mm to about 1 mm, e.g., about 0.2 mm, 0.3 mm, 0.4 mm, 0.5 mm, 0.6 mm, 0.7 mm, 0.8 mm, 0.9 mm, or 1 mm, e.g., from about 1 mm to about 5 mm, e.g., about 2 mm, 2.5 mm, 3 mm, 3.5 mm, 4 mm, 4.5 mmm, or 5 mm.

The thickness of the sheath may be from about 0.01 mm to about 1 mm, e.g., from about 0.01 mm to about 0.1 mm, e.g., about 0.02 mm, 0.03 mm, 0.04 mm, 0.05 mm, 0.06 mm, 0.07 mm, 0.08 mm, 0.09 mm, or 0.1 mm, e.g., from about 0.1 mm to about 1 mm, e.g., about 0.2 mm, 0.3 mm, 0.4 mm, 0.5 mm, 0.6 mm, 0.7 mm, 0.8 mm, 0.9 mm, or 1 mm. The thickness of the sheath may be substantially uniform throughout or may have different thicknesses in different portions or regions of the sheath.

The diameter of the conductor may be from about 50% to about 99% of the inner diameter of the sheath. For example, the diameter may be from about 55% to about 95%, about 60% to about 90%, about 65% to about 85%, 70% to about 80%, or about 75%.

The elongate conductor may be a substantially cylindrical (e.g., a cylindrical wire). A cross-section of the sheath may be substantially circular or elliptical. The diameter of the conductor may be from about 0.01 mm to about 5 mm, e.g., from about 0.1 mm to about 1 mm, e.g., from about 0.2 mm to about 0.5 mm, e.g., from about 0.2 mm to about 0.3 mm. For example, the inner diameter of the sheath may be from 0.01 mm to about 5 mm, e.g., from about 0.01 mm to about 0.1 mm, e.g., about 0.02 mm, 0.03 mm, 0.04 mm, 0.05 mm, 0.06 mm, 0.07 mm, 0.08 mm, 0.09 mm, or 0.1 mm, e.g., from about 0.1 mm to about 1 mm, e.g., about 0.2 mm, 0.3 mm, 0.4 mm, 0.5 mm, 0.6 mm, 0.7 mm, 0.8 mm, 0.9 mm, or 1 mm, e.g., from about 1 mm to about 5 mm, e.g., about 2 mm, 2.5 mm, 3 mm, 3.5 mm, 4 mm, 4.5 mmm, or 5 mm. In some embodiments, the diameter of the conductor is about 0.2 mm. In some embodiments, the elongate conductor has a diameter of from about 100 μm to about 200 μm. In some embodiments, the diameter of the elongate conductor is about 150 μm.

The diameter of the conductor may be substantially uniform throughout or may have different thicknesses in different portions or regions of the conductor.

In some embodiments, the substantially planar electrode is from about 2 mm to about 15 mm (e.g., about 2 mm, 3 mm, 4 mm, 5 mm, 6 mm, 7 mm, 8 mm, 9 mm, 10 mm, 11 mm, 12 mm, 13 mm, 14 mm, or 15 mm) in one or more dimensions (e.g., both dimensions) perpendicular to the longitudinal axis.

In some embodiments, the substantially planar electrode is substantially symmetrical about a longitudinal plane.

In some embodiments, the substantially planar electrode is convex.

In some embodiments, the elongate conductor is a wire, wherein the substantially planar electrode includes the distal portion of the wire.

In some embodiments, the distal portion of the wire includes a preformed angle (e.g., preformed right angle) on a longitudinal plane, wherein the preformed angle (e.g., preformed right angle) is between the substantially planar electrode and the proximal portion of the wire.

In some embodiments, the substantially planar electrode is a spiral. For example, the spiral may include about 1 to about 5 (e.g., 1, 2, 3, 4, or 5) revolutions about the longitudinal axis. In some embodiments, the spiral includes (e.g., consists of) 3 revolutions about the longitudinal axis. In some embodiments, the spiral includes (e.g., consists of) 2 revolutions about the longitudinal axis. For example, FIG. 3 depicts a spiral having 2 revolutions about its longitudinal axis.

In some embodiments, the substantially planar electrode extends no further than 1 mm (e.g., no further than 0.9 mm, 0.8 mm, 0.7 mm, 0.6 mm, 0.5 mm, 0.4 mm, 0.3 mm, 0.2 mm, or 0.1 mm) distal or proximal to the preformed angle (e.g., preformed right angle). In some embodiments, the substantially planar electrode extends no further than 1 mm (e.g., no further than 0.9 mm, 0.8 mm, 0.7 mm, 0.6 mm, 0.5 mm, 0.4 mm, 0.3 mm, 0.2 mm, or 0.1 mm) distal to the preformed angle (e.g., preformed right angle).

In some embodiments, the device includes nothing distal to the substantially planar electrode.

In some embodiments, the device is monopolar.

In some embodiments, the device is bipolar, wherein the device further includes an auxiliary electrode in electrical communication with the substantially planar electrode. The auxiliary electrode may be part of, or connected to, the sheath.

In some embodiments, the proximal portion of the elongate conductor is connected to a voltage source and/or a waveform controller.

In another aspect, the invention features a method of delivering an agent (e.g., an agent of interest, e.g., a therapeutic agent) into a target cell of a patient using the device as described herein. In some embodiments, the invention features a method of delivering an agent (e.g., an agent of interest (e.g., a therapeutic agent) or a sequence of interest (e.g., a therapeutic sequence)) into a target cell of a patient using the device as described herein. The method includes inserting a sheath (or a sheath comprising a needle) through an external tissue surface (e.g., sclera) of the subject and sliding the elongate conductor to the distal position to form the substantially planar electrode. The method further includes positioning the substantially planar electrode into electrical communication with a tissue interface separating the target cell from the substantially planar electrode. The method also includes transmitting one or more pulses of electric energy through the substantially planar electrode at conditions suitable for electrotransfer of the agent (e.g., therapeutic agent) into the target cell. In some embodiments in which the therapeutic agent is a nucleic acid vector (e.g., a non-viral nucleic acid vector, e.g., a naked nucleic acid vector, e.g., synthetic circular DNA vector), the nucleic acid is expressed by the target cell (e.g., a retinal cell, e.g., an RPE cell and/or a photoreceptor cell). Thus, methods of delivery described herein can likely be methods of expressing a sequence of interest (e.g., a therapeutic sequence).

In some embodiments, the substantially planar electrode is within about 10 mm (e.g., within 9 mm, 8 mm, 7 mm, 6 mm, 5 mm, 4.5 mm, 4 mm, 3.5 mm, 3 mm, 2.5 mm, 2 mm, 1.5 mm, 1 mm, 0.5 mm, 0.45 mm, 0.40 mm, 0.35 mm, 0.30 mm, 0.25 mm, 0.20 mm, 0.15 mm, 0.10 mm, 0.05 mm, or less) of the tissue interface. The substantially planar electrode may be from 0.05 mm to 5 mm (e.g., about 0.5 mm, 0.10 mm, 0.15 mm, 0.20 mm, 0.25 mm, 0.30 mm, 0.35 mm, 0.40 mm, 0.45 mm, 0.5 mm, 1 mm, 1.5 mm, 2 mm, 2.5 mm, 3 mm, 3.5 mm, 4 mm, 4.5 mm, or 5 mm) from the tissue interface upon transmission of the one or more pulses. In some embodiments, the substantially planar electrode is about 1 mm from the tissue interface upon transmission of the one or more pulses.

The target cell may be within about 5 mm (e.g., 4.5 mm, 4 mm, 3.5 mm, 3 mm, 2.5 mm, 2 mm, 1.5 mm, 1 mm, 0.5 mm, 0.45 mm, 0.4 mm, 0.35 mm, 0.30 mm, 0.25 mm, 0.20 mm, 0.15 mm, 0.10 mm, or 0.05 mm) from the tissue interface. For example, the target cell may be from about 0.01 mm to about 1 mm (e.g., from about 0.01 mm to about 0.1 mm, e.g., about 0.02 mm, 0.03 mm, 0.04 mm, 0.05 mm, 0.06 mm, 0.07 mm, 0.08 mm, 0.09 mm, or 0.1 mm, e.g., from about 0.1 mm to about 1 mm, e.g., about 0.2 mm, 0.3 mm, 0.4 mm, 0.5 mm, 0.6 mm, 0.7 mm, 0.8 mm, 0.9 mm, or 1 mm) from the tissue interface.

In some embodiments, the conditions suitable for electrotransfer of the agent (e.g., therapeutic agent) into the target cell include a field strength at the target cell from about 10 V/cm to about 1,500 V/cm (e.g., from about 10 V/cm to about 100 V/cm, e.g., about 10 V/cm, 20 V/cm, 30 V/cm, 40 V/cm, 50 V/cm, 60 V/cm, 70 V/cm, 80 V/cm, 90 V/cm, or 100 V/cm, e.g., from about 100 V/cm to about 1,000 V/cm, e.g., about 200 V/cm, 300 V/cm, 400 V/cm, 500 V/cm, 600 V/cm, 700 V/cm, 800 V/cm, 900 V/cm, or 1,000 V/cm, e.g., from about 1,000 V/cm to about 1,500 V/cm, e.g., about 1,110 V/cm, 1,200 V/cm, 1,300 V/cm, 1,400 V/cm, or 1,500 V/cm). In some embodiments, the field strength at the target cell is from 50 V/cm to 300 V/cm. In some embodiments, the field strength at the target cell is about 100 V/cm.

In some embodiments, 1-12 pulses (e.g., 1, 2, 3, 4, 5, 6, 7, 8, 9, 10, 11, or 12 pulses) of electric energy are transmitted. In some embodiments, 2-12 pulses (e.g., 2, 3, 4, 5, 6, 7, 8, 9, 10, 11, or 12 pulses) of electric energy are transmitted. In some embodiments, 3-12 pulses (e.g., 3, 4, 5, 6, 7, 8, 9, 10, 11, or 12 pulses) of electric energy are transmitted. In some embodiments, 4-12 pulses (e.g., 4, 5, 6, 7, 8, 9, 10, 11, or 12 pulses) of electric energy are transmitted.

In some embodiments, the total number of pulses of electrical energy are delivered within 1-20 seconds. For example, the total number of pulses of electrical energy may be delivered within 1, 2, 3, 4, 5, 6, 7, 8, 9, 10, 11, 12, 13, 14, 15, 16, 17, 18, 19, or 20 seconds. The pulses of electrical energy may be, e.g., square waveforms. In some embodiments, the pulses of electrical energy have an amplitude from 5 V to 1,500 V. For example, the pulses of electrical energy may have an amplitude from about 5 V to 500 V, from about 500 V to about 1,000 V, or from about 1,000 V to about 1,500 V. In some embodiments, the pulses of electrical energy have an amplitude from 5 V to 500 V. For example, the pulses of electrical energy may have an amplitude of about 100 V, 125 V, 150 V, 175 V, 200 V, 225V, 250 V, 275 V, 300 V, 325 V, 350 V, 375 V, 400 V, 425 V, 450 V, 475 V, or 500 V. In some embodiments, the pulses of electrical energy have an amplitude of from about 5 V to about 250 V.

In some embodiments, the conditions suitable for electrotransfer of the agent into the target cell include a voltage at the target cell from 5 V to 100 V (e.g., from 10 V to 80V, from 15 V to 70 V, from 20 V to 60 V, or from 30 V to 50 V; e.g., about 10 V, about 15 V, about 20 V, about 25 V, about 30 V, about 35 V, about 40 V, about 45 V, about 50 V, about 55 V, about 60 V, about 65 V, or about 70 V).

In some embodiments, each of the pulses is from about 1 ms to about 200 ms, e.g., about 1 ms to about 100 ms. For example, each of the pulses may be about 1 ms, 2 ms, 3 ms, 4 ms, 5 ms, 6 ms, 7 ms, 8 ms, 9 ms, 10 ms, 20 ms, 30 ms, 40 ms, 50 ms, 60 ms, 70 ms, 80 ms, 90 ms, 100 ms, 110 ms, 120 ms, 130 ms, 140 ms, 150 ms, 160 ms, 170 ms, 180 ms, 190 ms, or 200 ms. In some embodiments, each of the pulses is from about 50 ms.

In some embodiments, the agent (e.g., therapeutic agent) has been previously administered to the tissue. In other embodiments, the method further includes administering the agent (e.g., therapeutic agent). The agent (e.g., therapeutic agent) may be administered concurrently or consecutively with one or more of the pulses.

In any of the aforementioned embodiments, the agent (e.g., therapeutic agent) may be a nucleic acid (e.g., a non-viral nucleic acid, e.g., a non-viral particulate nucleic acid or a naked nucleic acid). The nucleic acid may be DNA or RNA (e.g., circular DNA or circular RNA).

In some embodiments, the target cell is a retinal cell. The retinal cell may be, e.g., a retinal pigment epithelial (RPE) cell, a photoreceptor cell, or a ganglion cell.

In some embodiments, therapeutic agent is administered intravitreally, subretinally, or topically on the eye.

In some embodiments, the therapeutic agent is administered suprachoroidally.

### BRIEF DESCRIPTION OF THE DRAWINGS

FIG. 1 is a cross-sectional anatomical illustration of an eye, which shows structures relevant to the methods described herein.

FIGS. 2A-2D are drawings showing methods for delivering a therapeutic agent to a target retinal cell of an individual. White lines represent flow of current. FIG. 2A illustrates an intravitreal pulsed electric field transmission in which a positive voltage is transmitted by a monopolar needle electrode in the vitreous humor near the target retinal cells. FIG. 2B illustrates a subretinal pulsed electric field transmission in which a negative voltage is transmitted by a monopolar needle electrode in the subretinal space (e.g., in the bleb) at or near the target retinal cells. FIG. 2C illustrates an intravitreal pulsed electric field transmission in which a positive voltage is transmitted by a monopolar planar electrode in the vitreous humor near the target retinal cells. FIG. 2D illustrates an intravitreal pulsed electric field transmission in which a negative voltage is transmitted by a monopolar planar electrode in the vitreous humor near the target retinal cells.

FIGS. 3A-3E are drawings showing a method for suprachoroidally delivering a therapeutic agent to a target retinal cell of an individual. White lines represent flow of current. FIG. 3A illustrates a suprachoroidal injection of a pharmaceutical composition. A white arrow shows a path of distribution of the pharmaceutical composition upon injection, throughout the suprachoroidal space toward a posterior region of the eye (i.e., toward the target retinal cells, e.g., toward the macula). FIG. 3B illustrates an intravitreal pulsed electric field transmission in which a positive voltage is transmitted by a monopolar needle electrode in the vitreous humor near the target retinal cells. FIG. 3C illustrates an intravitreal pulsed electric field transmission in which a negative voltage is transmitted by a monopolar needle electrode in the vitreous humor near the target retinal cells. FIG. 3D illustrates an intravitreal pulsed electric field transmission in which a positive voltage is transmitted by a planar needle electrode in the vitreous humor near the target retinal cells. FIG. 3E illustrates an intravitreal pulsed electric field transmission in which a negative voltage is transmitted by a planar needle electrode in the vitreous humor near the target retinal cells.

FIGS. 4A and 4B are schematic drawings showing a device as described herein. FIG. 4A shows a cross-section of the device with a sheath and the elongate conductor in a retracted position, such that the distal portion of the conductor is substantially straight. Also shown is an insulator between the elongate conductor and the sheath. FIG. 4B shows the device with the elongate conductor in a deployed position, such that the distal portion of the elongate conductor is relaxed radially to form a substantially planar electrode perpendicular to the longitudinal axis of the sheath.

FIG. 5 is a schematic drawing of a bipolar device with an elongate conductor in a deployed position, such that the distal portion of the elongate conductor is relaxed radially to form a substantially planar electrode perpendicular to the longitudinal axis of the sheath. An auxiliary electrode is present on the device at the outside surface of the sheath, proximal to the distal end of the sheath.

FIG. 6 is a schematic drawing showing the substantially planar electrode in the deployed position. The elongate conductor is in a spiral shape with about two revolutions about the longitudinal axis. Also shown is an insulator between the elongate conductor and the sheath.

FIGS. 7A-7C are a series of electrical simulation plots representing voltage distribution (V/cm) over a transverse cross-section of an eye containing a needle electrode at the posterior portion of the vitreous humor. FIG. 7A shows the needle electrode offset from the vitreous humor-retina interface by 0.25 mm. FIG. 7B is an expanded view of a portion of FIG. 7A, showing detail of the vitreous humor-retina interface. FIG. 7C shows the needle electrode offset from the vitreous humor-retina interface by 0.95 mm.

FIGS. 8A-8C are a series of electrical simulation plots representing voltage distribution (V/cm) over a transverse cross-section of an eye containing a substantially planar electrode at the posterior portion of the vitreous humor. FIG. 8A shows the needle electrode offset from the vitreous humor-retina interface by 0.95 mm. FIG. 8B is an expanded view of a portion of FIG. 8A, showing detail of the vitreous humor-retina interface. FIG. 8C shows the substantially planar electrode offset from the vitreous humor-retina interface by 0.25 mm.

FIGS. 9A and 9B are a set of simulation plots representing voltage (e.g., potential) over a transverse cross-section of an eye containing a 20 V electrode at the posterior portion of the vitreous humor (0.4 mm from the vitreous humor-retina interface). FIG. 9A shows a needle electrode. FIG. 9B shows a spiral (substantially planar) electrode.

FIG. 10 is schematic drawing of a device having a handle and a slider in which the proximal end of the sheath is disposed at the surface of the distal end of the handle. The elongate conductor is disposed along the longitudinal axis within the handle and is attached to the slider.

FIG. 11 shows a schematic drawing of a device having a handle and a slider in which the proximal end of the sheath extends beyond the surface of the distal end of the handle and into a hollow region of the handle.

FIGS. 12A-12C are schematic drawings of a device with a handle and a slider. The handle is cylindrical and includes a cap at each of the distal and proximal ends. The slider fits within the handle and further includes a control member that moves the slider. FIG. 12A shows the device having a first sheath connected to the elongate conductor. The device further includes a second sheath connected to the slider. FIG. 12B shows an exploded view of the handle and the slider. The slider may include an internal element connected to the handle. FIG. 12C shows a perspective view of FIG. 12B.

FIG. 13 is a set of schematic drawings showing the dimensions of a cap positioned on the distal end of the slider. Units are shown in inches.

FIG. 14 is a set of schematic drawings showing the dimensions of a cap that is positioned on the proximal end of the slider. Units are shown in inches.

FIG. 15 is a set of schematic drawings showing the dimensions of an exemplary handle. Units are shown in inches.

FIG. 16 is a set of schematic drawings showing the dimensions a sheath (18-gauge hypodermic needle). Units are shown in inches.

FIG. 17 is a set of schematic drawings showing the dimensions of the control member of a handle. Units are shown in inches.

FIG. 18 is a schematic drawing showing the dimensions of an insulator (polyimide tube). Units are shown in inches.

FIG. 19 is a set of schematic drawings showing the dimensions of a slider. Units are shown in inches.

FIG. 20 is a set of schematic drawings showing the dimensions of a sheath (23-gauge hypodermic needle). Units are shown in inches.

FIGS. 21A and 21B are confocal scanning laser ophthalmoscopy (cSLO) images measuring GFP fluorescence in pig eyes after electrotransfer of GFP-expressing DNA. FIG. 21A shows fluorescence at baseline (before electrotransfer) from a nasal (left) or temporal (right) direction. FIG. 21B shows fluorescence at day 7 post-electrotransfer (terminal endpoint) from a nasal (left) or temporal (right) direction.

FIGS. 22A-22D are optical coherence tomography (OCT) images showing structural integrity and no detectable inflammation in pig eyes after electrotransfer of GFP-expressing DNA. FIGS. 22A and 22B are images from baseline (before electrotransfer). FIGS. 22C and 22D are images at day 7 post-electrotransfer (terminal endpoint) from a nasal or temporal direction.

FIGS. 23A and 23B are photomicrographs showing histology of an adult pig eye after electrotransfer of a synthetic circular DNA vector encoding GFP using a monopolar needle electrode positioned in the subretinal bleb for electrotransfer. FIG. 23A shows immunohistochemistry (IHC) where GFP expression (blue stain) is detected in both the photoreceptor (PR) layer and the retinal pigment epithelial (RPE) layer. Cone opsin is stained yellow. FIG. 23B shows H&E staining of the retina after electrotransfer, showing preservation of retinal cell architecture.

FIGS. 24A and 24B are photomicrographs showing IHC of an adult pig eye after electrotransfer of a synthetic circular DNA vector encoding GFP using a monopolar needle electrode, the distal end of which was positioned in the vitreous within 1 mm from the retina. In FIG. 24A, GFP is stained blue, and RPE65 is stained yellow. In FIG. 24B, GFP is stained blue, and cone opsin is stained yellow.

FIG. 25 is a photomicrograph showing IHC of an adult pig eye after electrotransfer of a synthetic circular DNA vector encoding GFP using a bipolar needle electrode, the distal end (negative electrode) being positioned in the subretinal bleb and the positive electrode on the needle proximal to the negative electrode being positioned in the vitreous. GFP is stained blue, and cone opsin is stained yellow.

FIGS. 26A and 26B are photomicrographs showing histology of an adult pig eye after administration of a synthetic circular DNA vector encoding GFP without electrotransfer. FIG. 26A shows IHC, where no significant GFP expression (blue stain) was observed. Cone opsin is stained yellow. FIG. 26B shows H&E staining of the retina.

FIGS. 27A and 27B are photomicrographs showing histology of an adult pig eye after mock electrotransfer of a PBS control. FIG. 27A shows IHC, where no GFP expression (blue stain) was observed detected. FIG. 27B shows H&E staining of the retina after electrotransfer, showing preservation of retinal cell architecture.

FIGS. 28A and 28B are photomicrographs showing IHC staining of an adult pig eye after electrotransfer of a synthetic circular DNA vector encoding GFP using a substantially planar electrode as shown in FIG. 6. FIG. 28A shows staining of GFP in blue and RPE in yellow. FIG. 28B shows staining of GFP in blue and cone opsin in yellow.

FIGS. 29A-29E are a set of photomicrographs showing a time course of GFP expression in cultured induced RPE cells. Each figure has four panels; the top left-hand panel in each figure shows GFP fluorescence in cells incubated with synthetic circular DNA vector encoding GFP in the absence of electrotransfer; the top right-hand panel in each figure shows GFP fluorescence in cells incubated with synthetic circular DNA vector encoding GFP in the presence of electrotransfer; the bottom left-hand panel in each figure is a brightfield image showing the morphology of induced retinal pigment epithelial (iRPE) cells incubated with synthetic circular DNA vector encoding GFP in the absence of electrotransfer; and the bottom right-hand panel in each figure is a brightfield image showing the morphology of iRPE cells incubated with synthetic circular DNA vector encoding GFP in the presence of electrotransfer. FIG. 29A shows cells at day 4 of the time course, FIG. 29B shows cells at day 21 of the time course, FIG. 29C shows cells at day 32 of the time course, FIG. 29D shows cells at day 40 of the time course, and FIG. 29E shows cells at day 49 of the time course.

FIG. 30 is a bar graph showing mRNA expression of a synthetic circular DNA vector encoding an ABCA4 transgene (C³-ABCA4) electrotransferred into pig eye in vivo, as measured by qPCR. Endogenous (endo) pig ABCA4 is shown for comparison. PBS was injected and mock electrotransferred using the same PEF conditions as a negative control. mRNA expression levels were quantified in the neuroretina (NR) and RPE/choroid (RPE/Cho).

FIG. 31 is a bar graph showing mRNA expression of a synthetic circular DNA vector encoding GFP and MYO7A transgene electrotransferred into two pig eyes in vivo, as measured by qPCR. Endogenous (endo) pig MYO7A is shown in each eye, for comparison. mRNA expression levels were quantified in the neuroretina (NR) and RPE/choroid (RPE/Cho).

FIGS. 32A and 32B are photomicrographs showing histology of a pig retina six days after electrotransfer of an 8,656 bp synthetic circular DNA vector encoding human ABCA4 (C³-ABCA4). FIG. 32A shows ABCA4 protein stained blue (indicated by solid arrows) and RPE65 stained brown (indicated by dashed arrows). FIG. 32B shows ABCA4 protein stained blue and rhodopsin stained yellow. Arrows indicate dual staining (green).

FIG. 33 is a photomicrograph showing histology of an adult pig retina after electrotransfer of C³-ABCA4. ABCA4 protein is stained blue (indicated by arrows).

FIG. 34 is a photomicrograph showing histology of a human retina (untreated). Endogenous ABCA4 protein is stained blue (indicated by arrows).

FIG. 35 is a photograph of a western blot showing ABCA4 protein expression in iRPE cells in vitro. Lane 1 is a negative control. Lanes 2-4 were loaded with sample from cells transfected with plasmid (lanes 2 and 3) or synthetic circular DNA vector (lanes 3 and 4). Transgenes were the same between plasmid and synthetic DNA vector between lanes 1 and 3, and between lanes 2 and 4.

FIGS. 36A-36F are photomicrographs showing fluorescence of iRPE cells after electroporation-mediated transfection of synthetic circular DNA encoding ABCA4 and plasmid encoding ABCA4 in vitro.

FIGS. 36A-36C show ZO-1/GFP (FIG. 36A), ABCAA4 (FIG. 36B), and overlayed ZO-1/GFP and ABCA4 (FIG. 36C) after transfection with synthetic circular DNA encoding ABCA4. FIGS. 36D-36F show ZO-1/GFP (FIG. 36D), ABCAA4 (FIG. 36E), and overlayed ZO-1/GFP and ABCA4 (FIG. 36F) after transfection with plasmid ABCA4.

FIG. 37 is a photograph of a western blot showing MYO7A protein expression in iRPE cells in vitro. Lane 1 was loaded with sample from cells transfected with plasmid encoding GFP. Lanes 2 and 3 were loaded with sample from cells transfected with plasmid encoding MYO7A. Lane 4 was loaded with sample from cells transfected with synthetic circular DNA vector encoding the same MYO7A transgene as Lane 3.

FIGS. 38A-38F are photomicrographs showing fluorescence of iRPE cells after electroporation-mediated transfection of synthetic circular DNA encoding MYO7A and plasmid encoding MYO7A in vitro.

FIGS. 38A-38C show ZO-1/GFP (FIG. 38A), MYO7A (FIG. 38B), and overlayed ZO-1/GFP and MYO7A (FIG. 38C) after transfection with synthetic circular DNA encoding MYO7A. FIGS. 38D-38F show ZO-1/GFP (FIG. 38D), MYO7A (FIG. 38E), and overlayed ZO-1/GFP and MYO7A (FIG. 38F) after transfection with plasmid MYO7A.

### DETAILED DESCRIPTION

Provided herein are therapeutic agents (and pharmaceutical compositions thereof) and methods of delivery thereof to ocular cells, such as retinal cells. Therapeutic agents (e.g., nucleic acid vectors encoding therapeutic proteins) can be delivered to ocular cells (e.g., retinal cells) by injection of the therapeutic agent and/or transmission of electrical energy (e.g., current) into the target tissue (e.g., retina). Thus, in some instances, approaches described herein involve electrotransfer, a process in which transmission of an electric field into an ocular tissue (e.g., retina) promotes delivery of the therapeutic agent (e.g., nucleic acid vector (e.g., non-viral DNA vectors e.g., circular DNA vector that lacks a bacterial original of replication, a drug resistance gene, and/or a recombination site)) into a target ocular cell (e.g., retinal cell, e.g., a photoreceptor and/or retinal pigment epithelial cell). Additionally, or alternatively, methods of the present invention involve administration of therapeutic agents (e.g., nucleic acid vectors (e.g., non-viral DNA vectors, e.g., circular DNA vectors that lack a bacterial original of replication, a drug resistance gene, and/or a recombination site)) to an individual. For example, in particular embodiments of the methods described herein, a therapeutic agent (e.g., nucleic acid vector (e.g., non-viral DNA vector, e.g., circular DNA vectors that lack a bacterial original of replication, a drug resistance gene, and/or a recombination site)) is delivered to a target cell (e.g., a retinal cell) by electrotransfer after it has been administered (e.g., by suprachoroidal administration) to the individual.

The present invention also features devices and methods for electrotransfer of a therapeutic agent into a target cell, such as a retinal cell (e.g., retinal pigment epithelial cell, photoreceptor cell, or ganglion cell). The device contains a sheath with a retractable elongate conductor that transfers electrical energy to the target cell through a substantially planar electrode. The device produces an electric field suited to the target tissue topography, increases the zone of cells exposed to an electric field, and can be more tolerant of misalignment than electrodes that lack a planar structure (e.g., conventional needle or wire electrodes). In turn, some embodiments of the device and methods of use thereof advantageously require lower voltage settings than, e.g., a needle or straight wire electrode. The device can provide improved transfection as the electrode produces an electric field that covers a greater depth and larger diameter of target tissue, relative to, e.g., a straight wire electrode. Furthermore, the electrode covers a larger volume than other devices, such as a wire electrode. The device is also not as sensitive to changes in position from the target tissue (e.g., the retina) as a wire electrode. Furthermore, by providing a rounded or spiral electrode, the device has an atraumatic interface with its target (e.g., retina) as opposed to a sharp feature pointing at the target.

### I. Definitions

Unless defined otherwise, technical and scientific terms used herein have the same meaning as commonly understood by one of ordinary skill in the art to which this invention belongs and by reference to published texts, which provide one skilled in the art with a general guide to many of the terms used in the present application. In the event of any conflicting definitions between those set forth herein and those of a referenced publication, the definition provided herein shall control.

As used herein, the terms “suprachoroid” and “suprachoroidal space,” are used interchangeably to refer to the space (or volume) and/or potential space (or potential volume) in the region of the eye between the sclera and choroid, bound anteriorly in the region of the scleral spur and posteriorly by the transscleral connections of the short posterior ciliary vessels to the choroid. The suprachoroidal space is primarily composed of closely packed layers of long pigmented processes derived from each of the two adjacent tissues; however, a space can develop in this region as a result of fluid or other material buildup in the suprachoroidal space and the adjacent tissues. The suprachoroidal space can be expanded by fluid buildup because of some disease state in the eye or as a result of some trauma or surgical intervention. In some embodiments, the fluid buildup is intentionally created by the injection of a pharmaceutical composition into the suprachoroidal space to create and/or expand further the suprachoroidal space.

As used herein, the term “microneedle” refers to a conduit body having a base, a shaft, and a. tip end suitable for insertion into the sclera and/or other ocular tissue and has dimensions suitable for minimally invasive insertion and drug formulation infusion as described herein. The length of a microneedle (i.e., the length of the shaft of the microneedle and the bevel height of the microneedle) does not exceed 2 mm and a diameter of the microneedle does not exceed 600 microns.

As used herein, “electrotransfer” refers to movement of a molecule (e.g., a nucleic acid, e.g., a naked nucleic acid) across a membrane of a target cell (e.g., from outside to inside the target cell, e.g., a retinal cell) that is caused by transmission of an electric field (e.g., a pulsed electric field) to the microenvironment in which the cell resides (e.g., the retina). Electrotransfer may occur as a result of electrophoresis, i.e., movement of a molecule (e.g., a nucleic acid, e.g., a naked nucleic acid) along an electric field (e.g., in the direction of current), based on a charge of the molecule. Electrophoresis can induce electrotransfer, for example, by moving a molecule (e.g., a nucleic acid, e.g., a naked nucleic acid) into proximity of a cell membrane to allow a biotransport process (e.g., endocytosis including pinocytosis or phagocytosis) or passive transport (e.g., diffusion or lipid partitioning) to carry the molecule into the cell. Additionally, or alternatively, electrotransfer may occur as a result of electroporation, i.e., generation of pores in the target cell caused by transmission of an electric field (e.g., a pulsed electric field), wherein the size, shape, and duration of the pores are suitable to accommodate movement of a molecule (e.g., a nucleic acid, e.g., a naked nucleic acid) from outside the target cell to inside the target cell. Thus, in some instances, electrotransfer occurs as a result of a combination of electrophoresis and electroporation.

As used herein, the term “relax,” and grammatical derivations thereof, refers to a change in shape of a structure from a constrained shape to an unconstrained shape, which is driven by unloading of elastic potential energy. A shape memory material (e.g., shape memory alloy, e.g., NiTi) can relax into a preformed shape upon removal of a structural constraint. For example, a preformed shape memory wire housed in a rigid sheath is straight until it is unsheathed, at which point the shape memory material relaxes into its preformed shape.

As used herein, a “spiral” refers to the path of a point in a plane moving around a central point while receding from or approaching it.

As used herein, a “substantially planar electrode” refers to an electrode in which two of its perpendicular dimensions (e.g., Cartesian dimensions, e.g., depth and width) are each at least twice its third perpendicular dimension (e.g., length). In some embodiments, a substantially planar electrode refers to an electrode in which two of its perpendicular dimensions are each at least 3 times, at least 4 times, at least 5 times, at least 6 times, at least 7 times, at least 8 times, at least 9 times, at least 10 times, at least 20 times, at least 30 times, at least 40 times, at least 50 times, at least 60 times, at least 70 times, at least 80 times, or at least 100 times, or greater than its third perpendicular dimension.

As used herein, the term “circular DNA vector” refers to a DNA molecule in a circular form. Such circular form is typically capable of being amplified into concatamers by rolling circle amplification. A linear double-stranded nucleic acid having conjoined strands at its termini (e.g., covalently conjugated backbones, e.g., by hairpin loops or other structures) is not a circular vector, as used herein. The term “circular DNA vector” is used interchangeable herein with the term “covalently closed and circular DNA vector.” A skilled artisan will understand that such circular vectors include vectors that are covalently closed with supercoiling and complex DNA topology, as is described herein. In some embodiments, the circular DNA vector is not supercoiled (e.g., open circular). In particular embodiments, a circular DNA vector is supercoiled. In certain instances, a circular DNA vector lacks a bacterial origin of replication.

As used herein, a “cell-free” method of producing a circular DNA vector refers to a method that does not rely on containment of any of the DNA within a host cell, such as a bacterial (e.g., E. coli) host cell, to facilitate any step of the method, from providing the template DNA vector (e.g., plasmid DNA vector) through producing the circular DNA vector. For example, a cell-free method occurs within one or more synthetic containers (e.g., glass or plastic tubes, bioreactors, vessels, tanks, or other suitable containers) within appropriate solutions (e.g., buffered solutions), to which enzymes and other agents may be added to facilitate DNA amplification, modification, and isolation. Cell-free production methods may use template DNA that has been produced within cells.

As used herein, the term “recombination site” refers to a nucleic acid sequence that is a product of site-specific recombination, which includes a first sequence that corresponds to a portion of a first recombinase attachment site and a second sequence that corresponds to a portion of a second recombinase attachment site. One example of a hybrid recombination site is attR, which is a product of site-specific recombination and includes a first sequence that corresponds to a portion of attP and a second sequence that corresponds to a portion of attB. Alternatively, recombination sites can be generated from Cre/Lox recombination. Thus, a vector generated from Cre/Lox recombination (e.g., a vector including a LoxP site) includes a recombination site, as used herein. Other site-specific recombination events that generate recombination sites involve, e.g., lambda integrase, FLP recombinase, and Kw recombinase. Nucleic acid sequences that result from non-site-specific recombination events (e.g., ITR-mediated intermolecular recombination) are not recombination sites, as defined herein.

As used herein, the term “protein” refers to a plurality of amino acids attached to one another through peptide bonds (i.e., as a primary structure), including multimeric (e.g., dimeric, trimeric, etc.) proteins that are non-covalently associated (e.g., proteins having quaternary structure). Thus, the term “protein” encompasses peptides, native proteins, recombinant proteins, and fragments thereof. In some embodiments, a protein has a primary structure and no secondary, tertiary, or quaternary structure in physiological conditions. In some embodiments, a protein has a primary and secondary structure and no tertiary or quaternary structure in physiological conditions. In particular embodiments, a protein has a primary structure, a secondary structure, and a tertiary structure, but no quaternary structure in physiological conditions (e.g., a monomeric protein having one or more folded alpha-helices and/or beta sheets). In some embodiments, any of the proteins described herein have a length of at least 25 amino acids (e.g., 50 to 1,000 amino acids).

The terms “therapeutic sequence,” “therapeutic gene” and “heterologous gene” are used interchangeably to refer to a transgene to be administered (e.g., as part of a DNA vector or self-replicating RNA molecule). A therapeutic gene can be a mammalian gene encoding a protein that is endogenously expressed by the individual receiving the therapeutic gene or a protein that replaces a non-functional mutant protein expressed by the individual.

As used herein, the terms “disorder associated with a mutation,” “mutation associated with a disorder,” or protein or gene “-associated” disorder (e.g., ABCA4-associated retinal dystrophy) refer to a correlation between a disorder and the mutation in the gene or protein. In some embodiments, a disorder associated with a mutation is known or suspected to be wholly or partially, or directly or indirectly, caused by the mutation. For example, a subject having the mutation may be at risk of developing the disorder, and the risk may additionally depend on other factors, such as other (e.g., independent) mutations (e.g., in the same or a different gene), or environmental factors.

The term “ABCA4” refers to any native ABCA4 from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, splice variants, and fragments thereof. Functionally equivalent and improved variants can be determined on the basis of known ABCA4 signaling. ABCA4 encompasses full-length, unprocessed ABCA4, as well as any form of ABCA4 that results from native processing in the cell. An exemplary human ABCA4 sequence is provided as National Center for Biotechnology Information (NCBI) Reference Sequence: NG 009073. In some instances, the ABCA4 is encoded by a therapeutic gene having at least 95% sequence identity to SEQ ID NO: 16 (e.g., at least 96%, at least 97%, at least 98%, at least 99% or 100% sequence identity to SEQ ID NO: 16). In some instances, the ABCA4 is encoded by a therapeutic gene having at least 95% sequence identity to SEQ ID NO: 17 (e.g., at least 96%, at least 97%, at least 98%, at least 99% or 100% sequence identity to SEQ ID NO: 17). In some instances, the ABCA4 protein has at least 95% sequence identity to SEQ ID NO: 18 (e.g., at least 96%, at least 97%, at least 98%, at least 99% or 100% sequence identity to SEQ ID NO: 18).

The term “MYO7A” refers to any native MYO7A (also known as DFNB2, MYU7A, NSRD2, USH1B, DFNA11, or MYOVIIA) from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, splice variants, and fragments thereof. Functionally equivalent and improved variants can be determined on the basis of known MYO7A signaling. MYO7A encompasses full-length, unprocessed MYO7A, as well as any form of MYO7A that results from native processing in the cell. An exemplary human MYO7A sequence is provided as National Center for

Biotechnology Information (NCBI) Gene ID: 4647. In some instances, the MYO7A is encoded by a therapeutic gene having at least 95% sequence identity to any one of SEQ ID NO: 1 (e.g., at least 96%, at least 97%, at least 98%, at least 99% or 100% sequence identity to SEQ ID NO: 1). In some instances, the MYO7A encoded by the therapeutic gene has at least 95% sequence identity to any one of SEQ ID NOs: 2-9 (e.g., at least 96%, at least 97%, at least 98%, at least 99%, or 100% sequence identity to any one of SEQ ID NOs: 2-9).

The term “bestrophin 1 (BEST1)” refers to any native BEST1 (also known as ARB, BMB, BEST, RP50, VMD2, TU15B, or Best1V1Delta2) from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, splice variants, and fragments thereof. Functionally equivalent and improved variants can be determined on the basis of known BEST1 signaling (e.g., Ca²⁺ signaling in RPE cells). BEST1 encompasses full-length, unprocessed BEST1, as well as any form of BEST1 that results from native processing in the cell. An exemplary human BEST1 sequence is provided as National Center for Biotechnology Information (NCBI) Gene ID: 7439. In some instances, the BEST1 is encoded by a therapeutic gene having at least 95% sequence identity to SEQ ID NO: 10 (e.g., at least 96%, at least 97%, at least 98%, at least 99% or 100% sequence identity to SEQ ID NO: 10). In some instances, the BEST1 encoded by the therapeutic gene has at least 95% sequence identity to any one of SEQ ID NOs: 11-13 (e.g., at least 96%, at least 97%, at least 98%, at least 99%, or 100% sequence identity to any one of SEQ ID NOs: 11-13).

The term “complement factor H (CFH)” refers to any native CFH (also known as FH, HF, HF1, HF2, HUS, FHL1, AHUS1, AMBP1, ARMD4, ARMS1, or CFHL3) from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, splice variants, and fragments thereof. Functionally equivalent and improved variants can be determined on the basis of known CFH signaling (e.g., Ca²⁺ signaling in RPE cells). CFH encompasses full-length, unprocessed CFH, as well as any form of CFH that results from native processing in the cell. An exemplary human CFH sequence is provided as National Center for Biotechnology Information (NCBI) Gene ID: 3075. In some instances, the CFH is encoded by a therapeutic gene having at least 95% sequence identity to SEQ ID NO: 14 (e.g., at least 96%, at least 97%, at least 98%, at least 99% or 100% sequence identity to SEQ ID NOs 14). In some instances, the CFH encoded by the therapeutic gene has at least 95% sequence identity to SEQ ID NO: 15 (e.g., at least 96%, at least 97%, at least 98%, at least 99%, or 100% sequence identity to SEQ ID NO: 15).

As used herein, a “variant” of a therapeutic gene, a replicase, or a fragment thereof, differs in at least one amino acid residue from the reference amino acid sequence, such as a naturally occurring amino acid sequence or an amino acid sequence. In this context, the difference in at least one amino acid residue may consist, for example, in a mutation of an amino acid residue to another amino acid, a deletion or an insertion. A variant may be a homolog, isoform, or transcript variant of a therapeutic protein or a fragment thereof as defined herein, wherein the homolog, isoform or transcript variant is characterized by a degree of identity or homology, respectively, as defined herein.

In some instances, a variant of a therapeutic gene, or a fragment thereof, includes at least one amino acid substitution (e.g., 1-100 amino acid substitutions, 1-50 amino acid substitutions, 1-20 amino acid substitutions, 1-10 amino acid substitutions, e.g., 1 amino acid substitution, 2 amino acid substitutions, 3 amino acid substitutions, 4 amino acid substitutions, 5 amino acid substitutions, 6 amino acid substitutions, 7 amino acid substitutions, 8 amino acid substitutions, 9 amino acid substitutions, or 10 amino acid substitutions). Substitutions in which amino acids from the same class are exchanged for one another are called conservative substitutions. In particular, these are amino acids having aliphatic side chains, positively or negatively charged side chains, aromatic groups in the side chains or amino acids, the side chains of which can form hydrogen bridges, e.g., side chains which have a hydroxyl function. By conservative constitution, e.g., an amino acid having a polar side chain may be replaced by another amino acid having a corresponding polar side chain, or, for example, an amino acid characterized by a hydrophobic side chain may be substituted by another amino acid having a corresponding hydrophobic side chain (e.g., serine (threonine) by threonine (serine) or leucine (isoleucine) by isoleucine (leucine)). In certain embodiments, a variant of a protein or a fragment thereof may be encoded by the nucleic acid according to the invention, wherein at least one amino acid residue of the amino acid sequence includes at least one conservative substitution compared to a reference sequence, such as the respective naturally occurring sequence.

In some instances, insertions, deletions, and/or non-conservative substitutions are also encompassed by the term variant, e.g., at those positions that do not cause a substantial modification of the three-dimensional structure of the protein. Modifications to a three-dimensional structure by insertion(s) or deletion(s) can readily be determined by a person of skill in the art, e.g., using CD spectra (circular dichroism spectra).

In order to determine the percentage to which two sequences (e.g., nucleic acid sequences, e.g., DNA, RNA, or amino acid sequences) are identical, the sequences can be aligned in order to be subsequently compared to one another. For this purpose, gaps can be inserted into the sequence of the first sequence and the component at the corresponding position of the second sequence can be compared. If a position in the first sequence is occupied by the same component as is the case at a corresponding position in the second sequence, the two sequences are identical at this position. The percentage, to which two sequences are identical, is a function of the number of identical positions divided by the total number of positions. The percentage to which two sequences are identical can be determined using a mathematical algorithm. A preferred, but not limiting, example of a mathematical algorithm, which can be used is the algorithm of Karlin et al. (1993), PNAS USA, 90:5873-5877 or Altschul et al. (1997), Nucleic Acids Res., 25:3389-3402. Such an algorithm can be integrated, for example, in the BLAST program. Sequences, which are identical to the sequences of the present invention to a certain extent, can be identified by this program.

As used herein, the term “isolated” means artificially produced and not integrated into a native host genome. For example, an isolated nucleic acid vector includes nucleic acid vectors that are encapsulated in a lipid envelope (e.g., a liposome) or a polymer matrix. In some embodiments, the term “isolated” refers to a DNA vector that is: (i) amplified in vitro (e.g., in a cell-free environment), for example, by rolling-circle amplification or polymerase chain reaction (PCR); (ii) recombinantly produced by molecular cloning; (iii) purified, as by restriction endonuclease cleavage and gel electrophoretic fractionation, or column chromatography; or (iv) synthesized by, for example, chemical synthesis. An isolated nucleic acid vector is one which is readily manipulable by recombinant DNA techniques well-known in the art. Thus, a nucleotide sequence contained in a vector in which 5′ and 3′ restriction sites are known or for which polymerase chain reaction (PCR) primer sequences have been disclosed is considered isolated, but a nucleic acid sequence existing in its native state in its natural host is not. An isolated nucleic acid vector may be substantially purified, but need not be.

As used herein, the term “naked” refers to a nucleic acid molecule (e.g., a non-viral DNA vector, e.g., a circular DNA vector that lacks a bacterial original of replication, a drug resistance gene, and/or a recombination site) that is not complexed with another agent (e.g., encapsulated within, conjugated to, or non-covalently bound to another agent). Thus, a nucleic acid within an envelope (e.g., a lipid envelope) or a matrix of covalently linked or non-covalently associated units (e.g., a synthetic polymer matrix or a peptide or protein matrix) is not a naked nucleic acid molecule, as used herein. Naked nucleic acid molecules may be co-formulated (e.g., in solution) with agents that are not complexed with the naked nucleic acid molecule, such as buffering agents. In some instances of the present invention, a pharmaceutical composition includes a naked circular DNA vector. One example of a naked DNA is a covalently closed circular DNA (C3-DNA) described herein.

As used herein, a “vector” refers to a nucleic acid molecule capable of carrying a sequence of interest (e.g., a therapeutic gene, a therapeutic sequence, or a heterologous gene) to which is it linked into a target cell in which the therapeutic gene can then be replicated, processed, and/or expressed in the target cell. After a target cell or host cell processes the sequence of interest (e.g., genome) of the vector, the sequence of interest (e.g., genome) is not considered a vector. One type of vector is a “plasmid”, which refers to a circular double stranded DNA loop containing a bacterial backbone into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors” or “expression vectors”). In general, expression vectors of utility in recombinant DNA techniques are often in the form of plasmids.

As used herein, a “target cell” refers to a cell that expresses a modulatory protein encoded by a therapeutic gene. In some embodiments, a target cell is a retinal cell. For example, in particular embodiments, a target cell is an RPE cell. In other embodiments, a target cell is a photoreceptor. In particular embodiments, RPE cells and photoreceptors are target cells.

As used herein, the term “individual” includes any mammal in need of the methods of treatment or prophylaxis described herein (e.g., a mammal having a retinal dystrophy). In some embodiments, the individual is a human. In other embodiments, the individual is a non-human mammal (e.g., a non-human primate (e.g., a monkey), a mouse, a pig, a rabbit, a cat, or a dog). The subject may be male or female.

In one embodiment, the individual has Usher syndrome type 1B. In some embodiments, the individual has a bestrophinoapthy associated with a Best1 dominant mutation or a BEST1 recessive mutation, e.g., autosomal recessive bestrophinopathy, Best's vitelliform macular dystrophy, BEST1 adult-onset vitelliform macular dystrophy, or autosomal dominant vitreoretinochoroidopathy. In some embodiments, the individual has age-related macular degeneration.

As used herein, an “effective amount” or “effective dose” of a therapeutic agent (e.g., a nucleic acid vector) or composition thereof refers to an amount sufficient to achieve a desired biological and/or pharmacological effect, e.g., when administered to the individual according to a selected administration form, route, and/or schedule. As will be appreciated by those of ordinary skill in this art, the absolute amount of a particular composition that is effective can vary depending on such factors as the desired biological or pharmacological endpoint, the agent to be delivered, the target tissue, etc. Those of ordinary skill in the art will further understand that an “effective amount” can be contacted with cells or administered to a subject in a single dose or through use of multiple doses. An effective amount of a composition to treat an ocular disease may slow or stop disease progression (e.g., visual function) increase partial or complete response (e.g., visual function), relative to a reference population, e.g., an untreated or placebo population, or a population receiving the standard of care treatment.

As used herein, “treatment” (and grammatical variations thereof such as “treat” or “treating”) refers to clinical intervention in an attempt to alter the natural course of the individual being treated, which can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and improved prognosis. In some embodiments, nucleic acid vectors (e.g., circular DNA vectors) of the invention are used to delay development of a disease or to slow the progression of a disease.

By “reduce or inhibit” is meant the ability to cause an overall decrease preferably of 20% or greater, more preferably of 50% or greater, and most preferably of 75%, 85%, 90%, 95%, or greater.

The terms “level of expression” or “expression level” are used interchangeably and generally refer to the amount of a polynucleotide or an amino acid product or protein in a biological sample (e.g., retina). “Expression” generally refers to the process by which gene-encoded information is converted into the structures present and operating in the cell. Therefore, according to the invention, “expression” of a gene may refer to transcription into a polynucleotide, translation into a protein, or post-translational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (for example, transfer and ribosomal RNAs).

As used herein, “delivering,” “to deliver,” and grammatical variations thereof, is meant causing an agent (e.g., a therapeutic agent) to access a target cell. The agent can be delivered by administration of the agent to an individual having the target cell (e.g., systemically or locally administering the agent) such that the agent gains access to the organ or tissue in which the target cell resides. Additionally, or alternatively, the agent can be delivered by applying a stimulus to a tissue or organ harboring the agent, wherein the stimulus causes the agent to enter the target cell. Thus, in some instances, an agent is delivered to a target cell by transmitting an electric field into a tissue harboring the agent at conditions suitable for electrotransfer of the agent into a target cell within the tissue.

As used herein, “administering” is meant a method of giving a dosage of a therapeutic agent (e.g., a nucleic acid vector (e.g., a non-viral DNA vector, e.g., a circular DNA vector that lacks a bacterial original of replication, a drug resistance gene, and/or a recombination site)) of the invention or a composition thereof (e.g., a pharmaceutical composition, e.g., a pharmaceutical composition including a nucleic acid vector) to an individual. The compositions utilized in the methods described herein can be administered intraocularly, for example, suprachoroidally. The compositions utilized in the methods described herein can be administered intraocularly, for example, intravitreally, subretinally, or periocularly. Additionally, or alternatively, the composition can be delivered intravenously, subcutaneously, intradermally, percutaneously, intramuscularly, intraarterially, intraperitoneally, intralesionally, intracranially, intraarticularly, intraprostatically, intrapleurally, intratracheally, intrathecally, intranasally, intravaginally, intrarectally, topically, peritoneally, subconjunctivally, intravesicularly, mucosally, intrapericardially, intraumbilically, orally, topically, transdermally, conjunctivally, subtenonly, intracamerally, subretinally, retrobulbarly, intracanalicularly, by inhalation, by injection, by implantation, by infusion, by continuous infusion, by localized perfusion bathing target cells directly, by catheter, by lavage, in cremes, or in lipid compositions. The compositions utilized in the methods described herein can be administered systemically. The method of administration can vary depending on various factors (e.g., the compound or composition being administered and the severity of the condition, disease, or disorder being treated).

To be “administered in combination with” refers to administration of multiple therapeutic components as part of the same therapeutic regimen. A therapeutic agent (e.g., a nucleic acid vector (e.g., a non-viral DNA vector, e.g., a circular DNA vector that lacks a bacterial original of replication, a drug resistance gene, and/or a recombination site)) of the invention can be administered in combination with a pulsed electric field therapy, e.g., as part of the same outpatient procedure or over the course of multiple days. Additionally, or alternatively, a nucleic acid vector (e.g., circular DNA vector) of the invention can be administered in combination with another therapeutic agent (e.g., as part of the same pharmaceutical composition or as separate pharmaceutical compositions, at the same time or at different times).

The terms “a” and “an” mean “one or more of.” For example, “a cell” is understood to represent one or more cells. As such, the terms “a” and “an,” “one or more of a (or an),” and “at least one of a (or an)” are used interchangeably herein.

As used herein, the term “about” refers to a value within ±10% variability from the reference value, unless otherwise specified.

### II. Therapeutic Agents and Compositions

The present invention involves therapeutic agents for treatment of ocular diseases and disorders. Any therapeutic agent suitable for treatment of ocular disease (e.g., retinal dystrophy) upon delivery to an ocular target cell (e.g., a retinal cell) is contemplated as part of the present invention. Such therapeutic agents include nucleic acid vectors (e.g., non-viral DNA vectors, e.g., circular DNA vectors that lack a bacterial original of replication, a drug resistance gene, and/or a recombination site), therapeutic proteins, small molecule drugs, and pharmaceutical compositions thereof. Exemplary nucleic acid vectors include circular DNA vectors (e.g., circular DNA vectors encoding therapeutic replacement proteins (e.g., proteins that replace proteins that are endogenously expressed in healthy cells), including ABCA4, MYO7A, BEST1, and CFH). Any of the nucleic acid vectors described herein can be part of pharmaceutical compositions in a pharmaceutically acceptable carrier.

Nucleic Acid Vectors Nucleic acid vectors of the invention include non-viral nucleic acid vectors (e.g., non-viral DNA vectors or non-viral RNA vectors, e.g., circular DNA vectors and circular RNA vectors). In particular instances, nucleic acid vectors (e.g., non-viral nucleic acid vectors) are naked nucleic acid vectors (e.g., naked DNA (e.g., naked circular DNA (e.g., synthetic circular DNA) or naked linear DNA (e.g., closed ended DNA or doggybone DNA)) or naked RNA (e.g., naked circular RNA).

Some embodiments of the present invention include circular DNA vectors. In some instances, circular DNA vectors useful to carry the therapeutic genes (e.g., therapeutic replacement genes) described herein can be plasmid DNA vectors. In particular instances of the present invention, circular DNA vectors differ from conventional plasmid DNA vectors in that they lack plasmid backbone elements (e.g., bacterial elements such as (i) a bacterial origin of replication and/or (ii) a drug resistance gene). In some embodiments, circular DNA vectors encoding any of the therapeutic genes (e.g., therapeutic replacement genes) described herein lack a recombination site (e.g., synthetic circular DNA vectors produced using a cell-free process). In alternative embodiments, circular DNA vectors described herein include a recombination site (e.g., minicircle DNA vectors).

Circular DNA vectors of the invention can persist intracellularly (e.g., in quiescent cells, such as post-mitotic cells) as episomes. Vectors provided herein can be devoid of bacterial plasmid DNA components, such as immunogenic components (e.g., immunogenic bacterial signatures (e.g., CpG motifs)) or components additionally or otherwise associated with reduced persistence (e.g., CpG islands). For example, in some embodiments, the vector contains DNA in which at least 50% (e.g., at least 60%, at least 70%, at least 80%, at least 90%, at least 95%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or essentially all) of the DNA lacks one or more elements of bacterial plasmid

DNA, such as immunogenic components (e.g., immunogenic bacterial signatures (e.g., CpG motifs)) or components additionally or otherwise associated with reduced persistence (e.g., CpG islands). In some embodiments, at least 50% (e.g., at least 60%, at least 70%, at least 80%, at least 90%, at least 95%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or essentially all) of the DNA lacks CpG methylation. In some embodiments, the vector contains DNA in which at least 50% (e.g., at least 60%, at least 70%, at least 80%, at least 90%, at least 95%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or essentially all) of the DNA lacks bacterial methylation signatures, such as Dam methylation and Dcm methylation. For example, in some embodiments, the vector contains DNA in which at least 50% (e.g., at least 60%, at least 70%, at least 80%, at least 90%, at least 95%, at least 97%, at least 98%, at least 99%, or essentially all) of the GATC sequences are unmethylated (e.g., by Dam methylase). Additionally, or alternatively, the vector contains DNA in which at least 50% (e.g., at least 60%, at least 70%, at least 80%, at least 90%, at least 95%, at least 97%, at least 98%, at least 99%, or essentially all) of the CCAGG sequences and/or CCTGG sequences are unmethylated (e.g., by Dcm methylase).

In some embodiments of any of the aforementioned vectors, the DNA vector is persistent in vivo (e.g., the circularity and non-bacterial nature (i.e., by in vitro (e.g., cell-free) synthesis) are associated with long-term transcription or expression of a therapeutic gene of the DNA vector). In some embodiments, the persistence of the circular DNA vector is from 5% to 50% greater, 50% to 100% greater, one-fold to five-fold, or five-fold to ten-fold (e.g., at least 5%, 10%, 20%, 30%, 40%, 50%, 75%, one-fold, two-fold, three-fold, four-fold, five-fold, six-fold, seven-fold, eight-fold, nine-fold, ten-fold, or more) greater than a reference vector (e.g., a circular vector produced in bacteria or having one or more bacterial signatures not present in the vector of the invention). In some embodiments, the circular DNA vector of the invention persists for one week to four weeks, from one month to four months, from four months to one year, from one year to five years, from five years to twenty years, or from twenty years to fifty years (e.g., at least cone week, at least two weeks, at least one month, at least four months, at least one year, at least two years, at least five years, at least ten years, at least twenty years, at least thirty years, at least forty years, or at least fifty years).

A circular DNA vector of the invention may include a promoter operably linked 5′ to a therapeutic gene (e.g., therapeutic replacement gene). A promoter is operably linked to a therapeutic gene (e.g., therapeutic replacement gene) if the promoter is capable of effecting transcription of that therapeutic gene (e.g., therapeutic replacement gene). Promoters that can be used as part of circular DNA vectors include constitutive promoters, inducible promoters, native-promoters, and tissue-specific promoters. Examples of constitutive promoters include a cytomegalovirus (CMV) promoter (optionally with the CMV enhancer), a retroviral Rous sarcoma virus (RSV) LTR promoter (optionally with the RSV enhancer), an SV40 promoter, a dihydrofolate reductase promoter, a β-actin promoter, a phosphoglycerol kinase (PGK) promoter, and an EF1-alpha promoter. In particular embodiments of the invention, the circular DNA vector includes a CMV promoter. In some embodiments, the circular DNA vector includes a CAG promoter.

Alternatively, circular DNA vectors of the invention include inducible promoters. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Examples of inducible promoters regulated by exogenously supplied promoters include zinc-inducible sheep metallothionine (MT) promoters, T7 polymerase promoter systems, ecdysone insect promoters, tetracycline-repressible systems, tetracycline-inducible systems, RU486-inducible systems, and rapamycin-inducible systems. Still other types of inducible promoters which may be useful in this context are those which are regulated by a specific physiological state, e.g., temperature, acute phase, a particular differentiation state of the cell, or in replicating cells only. Inducible promoters and inducible systems are available from a variety of commercial sources.

A circular DNA vector of the invention may also include a polyadenylation sequence 3′ to the self-replicating RNA molecule-encoding sequence. Useful polyadenylation sequences include elongated polyadenylation sequences of greater than 20 nt (e.g., greater than 25 nt, greater that 30 nt, greater than 35 nt, greater than 40 nt, greater than 50 nt, greater than 60 nt, greater than 70 nt, or greater than 80 nt, e.g., from 20 to 100 nt, from 30 to 100 nt, from 40 to 100 nt, from 50 to 100 nt, from 60 to 100 nt, from 70 to 100 nt, from 80 to 100 nt, from 100 to 200 nt, from 200 to 300 nt, or from 300 to 400 nt, or greater).

Circular DNA vectors that lack bacterial elements such as a DNA origin of replication and/or a drug resistance gene can persist in an individual longer than conventional DNA vectors (e.g., plasmids) and longer than naked RNA.

Circular DNA vectors can have various sizes and shapes. A circular DNA vector carrying a therapeutic gene (e.g., therapeutic replacement gene) of the invention can be from 2.5 kb to 20 kb in length (e.g., from 5 kb to 19 kb, from 6 kb to 18 kb, from 7 kb to 16 kb, from 8 kb to 14 kb, or from 9 kb to 12 kb in length, e.g., from 5 kb to 6 kb, from 6 kb to 7 kb, from 7 kb to 8 kb, from 8 kb to 9 kb, from 9 kb to 10 kb, from 10 kb to 11 kb, from 11 kb to 12 kb, from 12 kb to 13 kb, from 13 kb to 14 kb, from 14 kb to 15 kb, from 15 kb to 16 kb, from 16 kb to 18 kb, or from 18 kb to 20 kb in length, e.g., about 3 kb, about 4 kb, about 5 kb, about 6 kb, about 7 kb, about 8 kb, about 9 kb, about 10 kb, about 10.5 kb, about 11 kb, about 11.5 kb, about 12 kb, about 12.5 kb, about 13 kb, about 14 kb, about 15 kb, about 16 kb, about 17 kb, about 18 kb, about 19 kb, or about 20 kb in length).

Circular DNA vectors useful as part of the present invention can be readily synthesized through various means known in the art and described herein. For example, circular DNA vectors that lack plasmid backbone elements (e.g., bacterial elements such as (i) a bacterial origin of replication and/or (ii) a drug resistance gene) can be made using in-vitro (cell-free) methods, which can provide purer compositions relative to bacterial-based methods. Such in-vitro synthesis methods may involve use of phage polymerase, such as Phi29 polymerase, as a replication tool using, e.g., rolling circle amplification. Particular methods of in-vitro synthesis of circular DNA vectors are further described in International Patent Publication WO 2019/178500, which is incorporated herein by reference.

In some instances, the nucleic acid vector is a non-viral nucleic acid vector (e.g., the nucleic acid vector is not encapsulated within a viral capsid). Additionally, or alternatively, in some embodiments, the nucleic acid vector is not encapsulated in an envelope (e.g., a lipid envelope) or a matrix (e.g., a polymer matrix) and is not physically associated with (e.g., covalently or non-covalently bound to) a solid structure (e.g., a particulate structure) prior to and upon administration to the individual. In some embodiments, the nucleic acid vector is untethered to any adjacent nucleic acid vectors such that, in a solution of nucleic acid vectors, each nucleic acid vector is free to diffuse independently of adjacent nucleic acid vectors. In some embodiments, the nucleic acid vector is associated with another agent in liquid solution, such as a charge-altering molecule or a stabilizing molecule.

The nucleic acid vector may be a naked nucleic acid vector, i.e., not complexed with another agent (e.g., encapsulated within, conjugated to, or non-covalently bound to another agent). Naked nucleic acid molecules may be co-formulated (e.g., in solution) with agents that are not complexed with the naked nucleic acid molecule, such as buffering agents and/or agents that are generally recognized as safe (GRAS) by the U.S. Food and Drug Administration.

### Therapeutic Genes

Nucleic acid vectors described herein include a therapeutic gene, such as a therapeutic gene or therapeutic sequence encoding a therapeutic replacement protein. A therapeutic replacement protein can replace a protein that is endogenously expressed in a healthy cell, e.g., a healthy retinal cell, or a non-functional mutant protein expressed by the individual being treated. Thus, it will be appreciated that the present nucleic acid vectors encoding therapeutic replacement proteins can be administered as gene replacement therapies and/or gene augmentation therapies.

Therapeutic genes of the present invention include ocular genes (e.g., genes encoding proteins expressed in ocular tissues, such as the retina). In some embodiments, the therapeutic sequence (e.g., therapeutic gene) is selected from the group consisting of MYO7A, BEST1, CFH, CEP290, USH2A, ADGRV1, CDH23, CRB1, PCDH15, RPGR, ABCA4, ABCC6, RIMS1, LRPS, CC2D2A, TRPM1, C3, IFT172, COL11A1, TUBGCP6, KIAA1549, CACNA1F, SNRNF200, PRPF8, VCAN, USH2A, HMCN1, RPE65, NR2E3, NRL, RHO, RP1, RP2, or OFD1. In some embodiments, the therapeutic sequence (e.g., therapeutic gene) is an autosomal dominant gene. In some embodiments, the therapeutic sequence (e.g., therapeutic gene) is an autosomal recessive gene. In some embodiments, the therapeutic sequence (e.g., therapeutic gene) is an X-linked gene.

In some embodiments, therapeutic protein encoded by the nucleic acid vector is a retinal pigment epithelium-specific protein, adrenoceptor alpha 2A, amyloid beta (A4) precursor protein, complement component 3, complement component 5, complement factor D (adipsin), thrombospondin receptor, complement component 5 receptor 1, HIF1 A, nerve growth factor receptor, STAT3, VEGFA, PDGFR, VEGFR½, plasminogen, tyrosine kinase, mTOR, Factor III, cadherin, chemokine receptor (¾), integrin A5, placental growth factor, protein tyrosine phosphatase, S1 PR1, vRaf, TGF-beta, HtrA serine peptidase 1, TNF receptor 10A, NOTCH4, insulin-like growth factor-binding protein 7, Ras responsive element binding protein 1, component factor H, component factor B, complement component 3, complement component 2, complement factor I, hepatic lipase, cholesteryl ester transfer protein, translocase of outer mitochondrial membrane 40, superoxide dismutase 2, mitochondrial, tenascin XB, collagen type X, alpha 1, myelin basic protein, collagen type VIII, alpha 1, bestrophin 1, carbohydrate (N-acetylglucosamine 6-0) sulfotransferase 6, retinitis pigmentosa GTPases, guanylate cyclase system (2D, A1A), calcium channels (A2, LA1 F), peripherin 2, cadherin 1, choroideremia (Rab escort protein 1), guanylate cyclase 2D, peripherin 2, mitochondrial encoded ATP synthase, mitochondrial encoded cytochromes, mitochondrial encoded NADH dehydrogenase, mitofusin 2, optic atrophy 1, three prime repair exonuclease 1, three prime repair exonuclease 1, DICER1, HIF-PHD, Hey 1, dominant negative CCR3, anti-Eotaxin mAb, Dcr1, Sema3E, VEGF-trap, PDGF-trap Nitrin1R, aA, aB Crystallin, Hey 2, a siruin, e.g., SIRT1, DR4-Fc, DR5-Fc, PD1R, RhoJ, sFLT-1, IGFR I-Fc, IGFBP7, PEDF, NPPB, CD59, PLEKHA1, RPE65, and/or PDE.

Nucleic acid vectors carrying these therapeutic sequences (e.g., therapeutic genes) are useful in the treatment of ocular diseases or disorders (e.g., retinal dystrophies associated with the transgene carried by the nucleic acid vector (e.g., ABCA4-associated retinal dystrophies, MYO7A-associated retinal dystrophies, or BEST1-associated retinal dystrophies), including Usher syndrome (e.g., Usher syndrome type 1B), retinitis pigmentosa (RP), diabetic ocular disorders (e.g., diabetic retinopathy or diabetic macular edema), dry eye, cataracts, retinal vein occlusion (e.g., central retinal vein occlusion or branched retinal vein occlusion), retinal artery occlusion, macular edema (e.g., macular edema occurring after retinal vein occlusion, macular degeneration (e.g., age related macular degeneration (AMD), wet macular degeneration (e.g., wet AMD), dry macular degeneration (e.g., dry AMD), or neovascular AMD), geographic atrophy, refraction and accommodation disorders, keratoconus, amblyopia, glaucoma, Stargardt disease, endophthalmitis, conjunctivitis, uveitis (e.g., posterior uveitis), retinal detachment, corneal ulcers, dacryocystitis, Duane retraction syndrome, optic neuritis, choroidal neovascularization, choroidal ischemia, or hypertensive retinopathy. Nucleic acid vectors carrying these therapeutic genes are useful in the treatment of symptoms of ocular diseases or disorders, such as any of the above diseases or disorders, or ocular symptoms of broader disorders, such as hypotension, hypertension, infection, sarcoid, or sickle cell disease. In some embodiments, a therapeutic gene is useful in the treatment of an acute disease. In other embodiments, the therapeutic gene is useful in the treatment of a chronic disease.

Other therapeutic sequences or genes (e.g., therapeutic genes encoding therapeutic proteins) useful within the nucleic acid vectors described herein include genes that encode a retinal protein other than any one or more of the proteins recited herein.

Therapeutic sequences or genes (e.g., therapeutic genes encoding a therapeutic replacement protein) of any of the nucleic acid vectors described herein may encode a functionally equivalent fragment of any of the proteins described herein, or variants thereof. A fragment of a protein or a variant thereof encoded by the nucleic acid vector according to the invention may include an amino acid sequence having a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99% (e.g., at least 70%, at least 80%, at least 85%, at least 90%, at least 95%, or at least 97% sequence identity) with a reference amino acid sequence (e.g., the amino acid sequence of the respective naturally occurring full-length protein or a variant thereof). In some embodiments, the therapeutic gene is selected from Table 1.

In any of the polycistronic nucleic acid vectors described herein, cleavage sites can be designed between protein-coding regions. For example, furin-P2A sites can separate any of the protein-coding genes described herein. Ribozymes can also be incorporated into an RNA molecule to cleave sites downstream of a protein-coding gene. In some embodiments, T2A, E2A, F2A, or any other suitable self-cleavage site (e.g., virus-derived cleavage site) can separate any of the protein-coding genes described herein.

In some embodiments, the therapeutic sequence (e.g., therapeutic gene) is greater than 4.5 Kb in length (e.g., the one or more therapeutic genes, together or each alone, are from 4.5 Kb to 25 Kb, from 4.6 Kb to 24 Kb, from 4.7 Kb to 23 Kb, from 4.8 Kb to 22 Kb, from 4.9 Kb to 21 Kb, from 5.0 Kb to 20 Kb, from 5.5 Kb to 18 Kb, from 6.0 Kb to 17 Kb, from 6.5 Kb to 16 Kb, from 7.0 Kb to 15 Kb, from 7.5 Kb to 14 Kb, from 8.0 Kb to 13 Kb, from 8.5 Kb to 12.5 Kb, from 9.0 Kb to 12.0 Kb, from 9.5 Kb to 11.5 Kb, or from 10.0 Kb to 11.0 Kb in length, e.g., from 4.5 Kb to 8 Kb, from 8 Kb to 10 Kb, from 10 Kb to 15 Kb, from 15 Kb to 20 Kb in length, or greater, e.g., from 4.5 Kb to 5.0 Kb, from 5.0 Kb to 5.5 Kb, from 5.5 Kb to 6.0 Kb, from 6.0 Kb to 6.5 Kb, from 6.5 Kb to 7.0 Kb, from 7.0 Kb to 7.5 Kb, from 7.5 Kb to 8.0 Kb, from 8.0 Kb to 8.5 Kb, from 8.5 Kb to 9.0 Kb, from 9.0 Kb to 9.5 Kb, from 9.5 Kb to 10 Kb, from 10 Kb to 10.5 Kb, from 10.5 Kb to 11 Kb, from 11 Kb to 11.5 Kb, from 11.5 Kb to 12 Kb, from 12 Kb to 12.5 Kb, from 12.5 Kb to 13 Kb, from 13 Kb to 13.5 Kb, from 13.5 Kb to 14 Kb, from 14 Kb to 14.5 Kb, from 14.5 Kb to 15 Kb, from 15 Kb to 15.5 Kb, from 15.5 Kb to 16 Kb, from 16 Kb to 16.5 Kb, from 16.5 Kb to 17 Kb, from 17 Kb to 17.5 Kb, from 17.5 Kb to 18 Kb, from 18 Kb to 18.5 Kb, from 18.5 Kb to 19 Kb, from 19 Kb to 19.5 Kb, from 19.5 Kb to 20 Kb, from 20 Kb to 21 Kb, from 21 Kb to 22 Kb, from 22 Kb to 23 Kb, from 23 Kb to 24 Kb, from 24 Kb to 25 Kb in length, or greater, e.g., about 4.5 Kb, about 5.0 Kb, about 5.5 Kb, about 6.0 Kb, about 6.5 Kb, about 7.0 Kb, about 7.5 Kb, about 8.0 Kb, about 8.5 Kb, about 9.0 Kb, about 9.5 Kb, about 10 Kb, about 11 Kb, about 12 Kb, about 13 Kb, about 14 Kb, about 15 Kb, about 16 Kb, about 17 Kb, about 18 Kb, about 19 Kb, about 20 Kb in length, or greater). In some embodiments, the therapeutic gene is greater than 2.5 Kb (e.g., between 2.5 Kb and 10 Kb, between 2.5 Kb and 8 Kb, or between 2.5 Kb and 6 Kb).

In some embodiments, the therapeutic sequence (e.g., therapeutic gene) is greater than 8 Kb (e.g., between 8 Kb and 15 Kb, between 8 Kb and 12 Kb, between 8 Kb and 10 Kb, or between 8 Kb and 9 Kb).

In some instances, a nucleic acid vector has a nucleic acid sequence driven by a CAG promoter that encodes an amino acid sequence having at least 95% sequence identity to SEQ ID NO: 18 (e.g., at least 96% sequence identity to SEQ ID NO: 18, at least 97% sequence identity to SEQ ID NO: 18, at least 98% sequence identity to SEQ ID NO: 18, at least 99% sequence identity to SEQ ID NO: 18, or 100% sequence identity to SEQ ID NO: 18). In some instances, the nucleic acid vector is a naked nucleic acid vector (e.g., a naked DNA vector, (e.g., a naked circular DNA vector (e.g., a plasmid DNA vector, a minicircle DNA vector, or a synthetic circular DNA vector lacking a recombination site (e.g., a supercoiled synthetic circular DNA vector)), a naked closed-ended DNA vector, or a naked doggybone DNA vector) or a naked RNA vector (e.g., a naked circular RNA vector)) comprising a nucleic acid sequence that encodes an amino acid sequence having at least 95% sequence identity to SEQ ID NO: 18 (e.g., at least 96% sequence identity to SEQ ID NO: 18, at least 97% sequence identity to SEQ ID NO: 18, at least 98% sequence identity to SEQ ID NO: 18, at least 99% sequence identity to SEQ ID NO: 18, or 100% sequence identity to SEQ ID NO: 18). In some instances, the nucleic acid vector (e.g., nonviral nucleic acid vector) comprises o a nucleic acid sequence that encodes an amino acid sequence having at least 95% sequence identity to SEQ ID NO: 18 (e.g., at least 96% sequence identity to SEQ ID NO: 18, at least 97% sequence identity to SEQ ID NO: 18, at least 98% sequence identity to SEQ ID NO: 18, at least 99% sequence identity to SEQ ID NO: 19, or 100% sequence identity to SEQ ID NO: 18), wherein the nucleic acid vector is encapsulated in a nanoparticle, a microparticle, a liposome, or a lipid nanoparticle.

Additionally, or alternatively, a nucleic acid vector includes a nucleic acid sequence having at least 95% sequence identity to SEQ ID NO: 19 (e.g., at least 96% sequence identity to SEQ ID NO: 19, at least 97% sequence identity to SEQ ID NO: 19, at least 98% sequence identity to SEQ ID NO: 19, at least 99% sequence identity to SEQ ID NO: 19, or 100% sequence identity to SEQ ID NO: 19). In some instances, the nucleic acid vector comprises or consists of the nucleic acid sequence of SEQ ID NO: 19. In some instances, the nucleic acid vector is a naked nucleic acid vector (e.g., a naked DNA vector, (e.g., a naked circular DNA vector (e.g., a plasmid DNA vector, a minicircle DNA vector, or a synthetic circular DNA vector lacking a recombination site (e.g., a supercoiled synthetic circular DNA vector)), a naked closed-ended DNA vector, or a naked doggybone DNA vector) or a naked RNA vector (e.g., a naked circular RNA vector)) comprising or consisting of a nucleic acid sequence having at least 95% sequence identity to SEQ ID NO: 19 (e.g., at least 96% sequence identity to SEQ ID NO: 19, at least 97% sequence identity to SEQ ID NO: 19, at least 98% sequence identity to SEQ ID NO: 19, at least 99% sequence identity to SEQ ID NO: 19, or 100% sequence identity to SEQ ID NO: 19). In some instances, the nucleic acid vector (e.g., nonviral nucleic acid vector) comprises or consists of a nucleic acid sequence having at least 95% sequence identity to SEQ ID NO: 19 (e.g., at least 96% sequence identity to SEQ ID NO: 19, at least 97% sequence identity to SEQ ID NO: 19, at least 98% sequence identity to SEQ ID NO: 19, at least 99% sequence identity to SEQ ID NO: 19, or 100% sequence identity to SEQ ID NO: 19), wherein the nucleic acid vector is encapsulated in a nanoparticle, a microparticle, a liposome, or a lipid nanoparticle.

Any of the aforementioned therapeutic sequences or therapeutic genes (e.g., therapeutic nucleic acid vectors, e.g., non-viral nucleic acid vectors, e.g., naked nucleic acid vectors, e.g., synthetic circular DNA vectors) can be used for the treatment of a disease or disorder (e.g., in an individual in need thereof, e.g., an individual having the disease or disorder or at risk of developing the disease or disorder). Thus, provided herein are uses of any of the aforementioned therapeutic sequences or therapeutic genes (e.g., therapeutic nucleic acid vectors, e.g., non-viral nucleic acid vectors, e.g., naked nucleic acid vectors, e.g., synthetic circular DNA vectors) for the treatment or prevention of any associated disorders according to any of the therapeutic methods and applications described herein (e.g., in Section III). Additionally, provided herein are any of the aforementioned therapeutic sequences or therapeutic genes (e.g., therapeutic nucleic acid vectors, e.g., non-viral nucleic acid vectors, e.g., naked nucleic acid vectors, e.g., synthetic circular DNA vectors) for use in the treatment or prevention of any associated disorders according to any of the therapeutic methods and applications described herein (e.g., in Section III).

### Pharmaceutical Compositions

The invention also provides methods involving administration of pharmaceutical compositions having a therapeutic agent (e.g., any of the nucleic acid vectors (e.g., circular DNA vectors) described herein) in a pharmaceutically acceptable carrier. For example, in some instances, the pharmaceutical composition administered in relation to the methods described herein includes a nucleic acid vector (e.g., e.g., a non-viral DNA vector, e.g., a circular DNA vector that lacks a bacterial original of replication, a drug resistance gene, and/or a recombination site) that encodes a therapeutic replacement protein that replaces a protein that is endogenously expressed in a healthy retinal cell and a pharmaceutically acceptable carrier.

In some instances, the pharmaceutical composition contains a non-viral nucleic acid vector (e.g., the pharmaceutical composition is substantially devoid of viral capsid). Additionally, or alternatively, the pharmaceutical composition may contain a nucleic acid vector that is not encapsulated in an envelope (e.g., a lipid envelope) or a matrix (e.g., a polymer matrix) and is not physically associated with (e.g., covalently or non-covalently bound to) a solid structure (e.g., a particulate structure) prior to and upon administration to the individual. In some embodiments of the pharmaceutical composition, the nucleic acid vector is untethered to any adjacent nucleic acid vectors such that, in a solution of nucleic acid vectors, each nucleic acid vector is free to diffuse independently of adjacent nucleic acid vectors. In some embodiments of the pharmaceutical composition, the nucleic acid vector is associated with another agent in liquid solution, such as a charge-altering molecule or a stabilizing molecule.

The pharmaceutical composition may contain the nucleic acid vector in naked form, i.e., the nucleic acid vector is not complexed with another agent (e.g., encapsulated within, conjugated to, or non-covalently bound to another agent). In such pharmaceutical compositions, naked nucleic acid molecules may be co-formulated (e.g., in solution) with agents that are not complexed with the naked nucleic acid molecule, such as buffering agents and/or agents that are generally recognized as safe (GRAS) by the U.S. Food and Drug Administration.

In some instances of the present invention, a pharmaceutical composition includes a naked circular DNA vector.

Pharmaceutically acceptable carriers may include excipients and/or stabilizers that are nontoxic to the individual at the dosages and concentrations employed. In some embodiments, the pharmaceutically acceptable carrier is an aqueous pH buffered solution. Examples of pharmaceutically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as tween, polyethylene glycol (PEG), and pluronics.

A pharmaceutical composition having a therapeutic agent of the invention (e.g., a nucleic acid vector, such as a circular DNA vector) may contain a pharmaceutically acceptable carrier. If the composition is provided in liquid form, the carrier may be water (e.g., pyrogen-free water), isotonic saline, or a buffered aqueous solution, e.g., a phosphate buffered solution or a citrate buffered solution. Injection of the pharmaceutical composition may be carried out in water or a buffer, such as an aqueous buffer, e.g., containing a sodium salt (e.g., at least 50 mM of a sodium salt), a calcium salt (e.g., at least 0.01 mM of a calcium salt), or a potassium salt (e.g., at least 3 mM of a potassium salt). According to a particular embodiment, the sodium, calcium, or potassium salt may occur in the form of their halogenides, e.g., chlorides, iodides, or bromides, in the form of their hydroxides, carbonates, hydrogen carbonates, or sulfates, etc. Without being limited thereto, examples of sodium salts include NaCl, Nal, NaBr, Na₂CO₂, NaHCO₂, and Na₂SO₄. Examples of potassium salts include, e.g., KCl, KI, KBr, K₂CO₂, KHCO₂, and K₂SO₄. Examples of calcium salts include, e.g., CaCl₂), Cal₂, CaBr₂, CaCO₂, CaSO₄, and Ca(OH)₂. Additionally, organic anions of the aforementioned cations may be contained in the buffer. According to a particular embodiment, the buffer suitable for injection purposes as defined above, may contain salts selected from sodium chloride (NaCl), calcium chloride (CaCl₂)) or potassium chloride (KCl), wherein further anions may be present. CaCl₂) can also be replaced by another salt, such as KCl. In some embodiments, salts in the injection buffer are present in a concentration of at least 50 mM sodium chloride (NaCl), at least 3 mM potassium chloride (KCl), and at least 0.01 mM calcium chloride (CaCl₂)). The injection buffer may be hypertonic, isotonic, or hypotonic with reference to the specific reference medium, i.e., the buffer may have a higher, identical or lower salt content with reference to the specific reference medium, wherein preferably such concentrations of the afore mentioned salts may be used, which do not lead to damage of cells due to osmosis or other concentration effects. Reference media are can be liquids such as blood, lymph, cytosolic liquids, other body liquids, or common buffers. Such common buffers or liquids are known to a skilled person. Ringer-Lactate solution is particularly preferred as a liquid basis.

One or more compatible solid or liquid fillers, diluents, or encapsulating compounds may be suitable for administration to a person. The constituents of the pharmaceutical composition according to the invention are capable of being mixed with the nucleic acid vector according to the invention as defined herein, in such a manner that no interaction occurs, which would substantially reduce the pharmaceutical effectiveness of the (pharmaceutical) composition according to the invention under typical use conditions. Pharmaceutically acceptable carriers, fillers and diluents can have sufficiently high purity and sufficiently low toxicity to make them suitable for administration to an individual being treated. Some examples of compounds which can be used as pharmaceutically acceptable carriers, fillers, or constituents thereof are sugars, such as lactose, glucose, trehalose, and sucrose; starches, such as corn starch or potato starch; dextrose; cellulose and its derivatives, such as sodium carboxymethylcellulose, ethylcellulose, cellulose acetate; powdered tragacanth; malt; gelatin; tallow; solid glidants, such as stearic acid, magnesium stearate; calcium sulfate; vegetable oils, such as groundnut oil, cottonseed oil, sesame oil, olive oil, corn oil and oil from theobroma; polyols, such as polypropylene glycol, glycerol, sorbitol, mannitol, and polyethylene glycol; or alginic acid.

The choice of a pharmaceutically acceptable carrier can be determined, according to the manner in which the pharmaceutical composition is administered.

Suitable unit dose forms for injection include sterile solutions of water, physiological saline, and mixtures thereof. The pH of such solutions may be adjusted to about 7.4. Suitable carriers for injection include hydrogels, devices for controlled or delayed release, polylactic acid, and collagen matrices. Suitable pharmaceutically acceptable carriers for topical application include those which are suitable for use in lotions, creams, gels and the like. If the pharmaceutical composition is to be administered perorally, tablets, capsules and the like are the preferred unit dose form.

Further additives which may be included in the pharmaceutical composition are emulsifiers, such as tween; wetting agents, such as sodium lauryl sulfate; coloring agents; pharmaceutical carriers; stabilizers; antioxidants; and preservatives.

The pharmaceutical composition according to the present invention may be provided in liquid or in dry (e.g., lyophilized) form. In a particular embodiment, the nucleic acid vector of the pharmaceutical composition is provided in lyophilized form. Lyophilized compositions including nucleic acid vector of the invention may be reconstituted in a suitable buffer, advantageously based on an aqueous carrier, prior to administration, e.g., Ringer-Lactate solution, Ringer solution, or a phosphate buffer solution. In certain embodiments of the invention, any of the nucleic acid vectors of the invention can be complexed with one or more cationic or polycationic compounds, e.g., cationic or polycationic polymers, cationic or polycationic peptides or proteins, e.g., protamine, cationic or polycationic polysaccharides, and/or cationic or polycationic lipids.

According to a particular embodiment, the nucleic acid vector of the invention may be complexed with lipids to form one or more liposomes, lipoplexes, or lipid nanoparticles. Therefore, in one embodiment, the inventive composition comprises liposomes, lipoplexes, and/or lipid nanoparticles comprising a therapeutic agent (e.g., a nucleic acid vector, e.g., a circular DNA vector).

Lipid-based formulations can be effective delivery systems for nucleic acid vectors due to their biocompatibility and their ease of large-scale production. Cationic lipids have been widely studied as synthetic materials for delivery of nucleic acids. After mixing together, nucleic acids are condensed by cationic lipids to form lipid/nucleic acid complexes known as lipoplexes. These lipid complexes are able to protect genetic material from the action of nucleases and deliver it into cells by interacting with the negatively charged cell membrane. Lipoplexes can be prepared by directly mixing positively charged lipids at physiological pH with negatively charged nucleic acids.

Conventional liposomes include of a lipid bilayer that can be composed of cationic, anionic, or neutral phospholipids and cholesterol, which encloses an aqueous core. Both the lipid bilayer and the aqueous space can incorporate hydrophobic or hydrophilic compounds, respectively. Liposome characteristics and behavior in-vivo can be modified by addition of a hydrophilic polymer coating, e.g., polyethylene glycol (PEG), to the liposome surface to confer steric stabilization. Furthermore, liposomes can be used for specific targeting by attaching ligands (e.g., antibodies, peptides, and carbohydrates) to its surface or to the terminal end of the attached PEG chains.

Liposomes are colloidal lipid-based and surfactant-based delivery systems composed of a phospholipid bilayer surrounding an aqueous compartment. They may present as spherical vesicles and can range in size from 20 nm to a few microns. Cationic lipid-based liposomes are able to complex with negatively charged nucleic acids via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the genetic material is then released from the endosome/carrier into the cytoplasm.

Cationic liposomes can serve as delivery systems for DNA and/or RNA. Cationic lipids, such as MAP, (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids to form nanoparticles by electrostatic interaction, providing high in vitro transfection efficiency. Furthermore, neutral lipid-based nanoliposomes for nucleic acid vector delivery as e.g., neutral 1,2-dioleoyl-sn-glycero-3-phosphatidylcholine (DOPC)-based nanoliposomes are available.

Thus, in one embodiment of the invention, the nucleic acid vector of the invention is complexed with cationic lipids and/or neutral lipids and thereby forms liposomes, lipid nanoparticles, lipoplexes or neutral lipid-based nanoliposomes.

In a particular embodiment, a pharmaceutical composition according to the invention comprises the nucleic acid vector of the invention that is formulated together with a cationic or polycationic compound and/or with a polymeric carrier. Accordingly, in a further embodiment of the invention, the nucleic acid vector as defined herein is associated with or complexed with a cationic or polycationic compound or a polymeric carrier, optionally in a weight ratio selected from a range of about 5:1 (w/w) to about 0.25:1 (w/w), e.g., from about 5:1 (w/w) to about 0.5:1 (w/w), e.g., from about 4:1 (w/w) to about 1:1 (w/w) or of about 3:1 (w/w) to about 1:1 (w/w), e.g., from about 3:1 (w/w) to about 2:1 (w/w) of nucleic acid vector to cationic or polycationic compound and/or with a polymeric carrier; or optionally in a nitrogen/phosphate (N/P) ratio of nucleic acid vector to cationic or polycationic compound and/or polymeric carrier in the range of about 0.1-10, e.g., in a range of about 0.3-4 or 0.3-1, e.g., in a range of about 0.5-1 or 0.7-1, e.g., in a range of about 0.3-0.9 or 0.5-0.9. For example, the N/P ratio of the nucleic acid vector to the one or more polycations is in the range of about 0.1 to 10, including a range of about 0.3 to 4, of about 0.5 to 2, of about 0.7 to 2 and of about 0.7 to 1.5.

The nucleic acid vectors described herein can also be associated with a vehicle, transfection or complexation agent for increasing the transfection efficiency and/or the expression of the modulatory gene according to the invention.

In some instances, the pharmaceutical composition contains a nucleic acid vector complexed with one or more polycations (e.g., protamine or oligofectamine), e.g., as a particle (e.g., a nanoparticle or microparticle). Further cationic or polycationic compounds that can be used as transfection agent, complexation agent, or particle (e.g., nanoparticle or microparticle) may include cationic polysaccharides, for example chitosan, polybrene, cationic polymers, e.g. polyethyleneimine (PEI), cationic lipids, e.g. DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPE, LEAP, DOPE: Dioleyl phosphatidylethanol-amine, DOSPA, DODAB, DOIC, DMEPC, DOGS: Dioctadecylamidoglicylspermin, DIMRI: Dimyristo-oxypropyl dimethyl hydroxyethyl ammonium bromide, MAP: dioleoyloxy-3-(trimethylammonio)propane, DC-6-14: 0,0-ditetradecanoyl-N-(α-trimethylammonioacetyl)diethanolamine chloride, CLIP1: rac-[(2,3-dioctadecyloxypropyl)(2-hydroxyethyl)]-dimethylammonium chloride, CLI P6: rac-[2(2,3-dihexadecyloxypropyl-oxymethyloxy)ethyl]trimethylammonium, CLI P9: rac-[2(2,3-dihexadecyloxypropyl-oxysuccinyloxy)ethyl]-trimethylammonium, oligofectamine, or cationic or polycationic polymers, e.g. modified polyaminoacids, such as β-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., polybetaaminoester (PBAE) or modified PBAE (e.g., polymers described in U.S. Pat. No. 8,557,231; PEGylated PBAE, such as those described in U.S. Patent Application No. 2018/0112038; any suitable polymer disclosed in Green et al., Acc. Chem. Res. 2008, 41(6): 749-759, such as diamine end modified 1,4 butanediol diacrylate-co-5-amino-1-pentanol polymers; any suitable modified PBAE as described in International Patent Publication No. WO 2020/077159 or WO 2019/070727; PBAE 457 as disclosed in Shen et al., Sci. Adv. 2020, 6: eaba1606, etc.), dendrimers, such as polypropylamine dendrimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., block polymers consisting of a combination of one or more cationic blocks (e.g. selected from a cationic polymer as mentioned above) and of one or more hydrophilic or hydrophobic blocks (e.g. polyethyleneglycole); etc.

In some instances, the pharmaceutical composition contains a nucleic acid vector encapsulated in a nanoparticle or microparticle, e.g., a biodegradable nanoparticle or microparticle (e.g., a cationic biodegradable polymeric nanoparticle or microparticle, such as PBAE or a modified PBAE (such as a polymer of formula (I) of International Patent Publication No. WO 2019/070727, or PBAE 457 as disclosed in Shen et al., Sci. Adv. 2020, 6: eaba1606), or a PEG-PBAE (or modified PBAE) copolymer) or a pH-sensitive nanoparticle or microparticle (e.g., a nanoparticle having a polymer of formula (I) of U.S. Pat. No. 10,792,374 (ECO)).

According to a particular embodiment, the pharmaceutical composition of the invention includes the therapeutic agent, e.g., nucleic acid vector (e.g., circular DNA vector) encapsulated within or attached to a polymeric carrier. A polymeric carrier used according to the invention might be a polymeric carrier formed by disulfide-crosslinked cationic components. The disulfide-crosslinked cationic components may be the same or different from each other. The polymeric carrier can also contain further components. It is also particularly preferred that the polymeric carrier used according to the present invention comprises mixtures of cationic peptides, proteins or polymers and optionally further components as defined herein, which are crosslinked by disulfide bonds as described herein. In this context, the disclosure of WO 2012/013326 is incorporated herewith by reference. In this context, the cationic components that form basis for the polymeric carrier by disulfide-crosslinkage are typically selected from any suitable cationic or polycationic peptide, protein or polymer suitable for this purpose, particular any cationic or polycationic peptide, protein or polymer capable of complexing the nucleic acid vector as defined herein or a further nucleic acid comprised in the composition, and thereby preferably condensing the nucleic acid vector. The cationic or polycationic peptide, protein or polymer, may be a linear molecule; however, branched cationic or polycationic peptides, proteins or polymers may also be used.

Every disulfide-crosslinking cationic or polycationic protein, peptide or polymer of the polymeric carrier, which may be used to complex the nucleic acid vector according to the invention included as part of the pharmaceutical composition of the invention may contain at least one SH moiety (e.g., at least one cysteine residue or any further chemical group exhibiting an SH moiety) capable of forming a disulfide linkage upon condensation with at least one further cationic or polycationic protein, peptide or polymer as cationic component of the polymeric carrier as mentioned herein.

Such polymeric carriers used to complex the nucleic acid vectors of the present invention may be formed by disulfide-crosslinked cationic (or polycationic) components. In particular, such cationic or polycationic peptides or proteins or polymers of the polymeric carrier, which comprise or are additionally modified to comprise at least one SH moiety, can be selected from proteins, peptides, and polymers as a complexation agent.

In other embodiments, the pharmaceutical composition according to the invention may be administered naked without being associated with any further vehicle, transfection, or complexation agent.

Any of the aforementioned pharmaceutical compositions (e.g., pharmaceutical compositions comprising any of the aforementioned therapeutic sequences or therapeutic genes (e.g., therapeutic nucleic acid vectors, e.g., non-viral nucleic acid vectors, e.g., naked nucleic acid vectors, e.g., synthetic circular DNA vectors)) can be used for the treatment of a disease or disorder (e.g., in an individual in need thereof, e.g., an individual having the disease or disorder or at risk of developing the disease or disorder). Thus, provided herein are uses of any of the aforementioned pharmaceutical compositions for the treatment or prevention of any associated disorders according to any of the therapeutic methods and applications described herein (e.g., in Section III). Additionally, provided herein are any of the aforementioned pharmaceutical compositions for use in the treatment or prevention of any associated disorders according to any of the therapeutic methods and applications described herein (e.g., in Section III).

### III. Therapeutic Methods and Applications

Provided herein are methods of delivering therapeutic agents (e.g., nucleic acid vectors encoding therapeutic replacement proteins) to ocular cells of an individual (e.g., a human patient). Such approaches may involve (a) electrotransfer to promote delivery of the therapeutic agent to a target cell in an individual, (b) administration of the therapeutic agent to the individual, or both (a) and (b). Such methods involve administration of any of the therapeutic agents or pharmaceutical compositions described herein, such as nucleic acid vectors or pharmaceutical compositions thereof (e.g., a pharmaceutical composition containing a naked nucleic acid vector). Also provided herein are methods of treating an ocular disease or disorder in an individual by a) electrotransfer to promote delivery of the therapeutic agent to a target cell in an individual, (b) administration of the therapeutic agent to the individual, or both (a) and (b). Particular ocular diseases that can be treated using such compositions and methods include ABCA4-associated retinal dystrophies (e.g., Stargardt disease), MYO7A-associated retinal dystrophies (e.g., Usher syndrome type 1B), bestrophinopathies associated with a BEST1 dominant mutation or BEST1 recessive mutation (e.g., autosomal recessive bestrophinopathy, Best's vitelliform macular dystrophy, BEST1 adult-onset vitelliform macular dystrophy, or autosomal dominant vitreoretinochoroidopathy), and age-related macular degeneration.

### Ocular Diseases and Individuals

Therapeutic agents and pharmaceutical compositions described herein can be used for treatment of various ocular diseases or disorders. In some instances, the ocular disease or disorder is a retinal disease or disorder, such as a retinal dystrophy (e.g., a retinal dystrophy characterized by reduced level of functional expression (e.g., a lack of functional expression) of a retinal protein in the individual relative to a reference (e.g., a healthy level of functional expression)). In some embodiments, the ocular disease or disorder (e.g., retinal disease or disorder) is a monogenic disorder. In some embodiments, the ocular disease or disorder (e.g., retinal disease or disorder) is a recessively inherited disorder. In some embodiments, the individual has, or is expected to develop, an ocular disease or disorder (e.g., retinal disease or disorder) caused by a heterozygous mutation. In other embodiments, the individual has, or is expected to develop, an ocular disease or disorder (e.g., retinal disease or disorder) caused by a homozygous mutation.

In some embodiments (e.g., in embodiments in which the individual is being treated for an ABCA4-associated retinal dystrophy, e.g., Stargardt disease or rod-cone dystrophy), the retinal protein is ABCA4. In such embodiments, the individual may be an adult, a teenager, or a child with retinal degeneration due to ABCA4 mutation (e.g., a biallelic ABCA4 mutation). In some instances, the individual has macular degeneration due to recessive biallelic ABCA4 mutations. The individual may have retinal degeneration of any severity due to biallelic ABCA4 mutations.

In some embodiments (e.g., in embodiments in which the individual is being treated for Usher syndrome 1B), the retinal protein is MYO7A.

In some embodiments (e.g., in embodiments in which the individual is being treated for a bestrophinopathy associated with a BEST1 dominant mutation or a BEST1 recessive mutation, e.g., autosomal recessive bestrophinopathy, Best vitelliform macular dystrophy, autosomal dominant vitreoretinochoroidopahy, BEST1 adult-onset vitelliform macular dystrophy, autosomal dominant microcornea, rod-cone dystrophy, early-onset cataract posterior staphyloma syndrome, or retinitis pigmentosa), the retinal protein is BEST1.

In some embodiments (e.g., in embodiments in which the individual is being treated for age-related macular degeneration), the retinal protein is CFH.

In some embodiments, the ocular disease or disorder is selected from the group consisting of Usher syndrome (e.g., Usher syndrome type 1B), autosomal recessive bestrophinopathy, autosomal dominant Best vitelliform macular dystrophy, macular degeneration (e.g., age related macular degeneration (AMD), wet macular degeneration (e.g., wet AMD), dry macular degeneration (e.g., dry AMD), or neovascular AMD), geographic atrophy, retinitis pigmentosa (RP), diabetic ocular disorders (e.g., diabetic retinopathy or diabetic macular edema), dry eye, cataracts, retinal vein occlusion (e.g., central retinal vein occlusion or branched retinal vein occlusion), retinal artery occlusion, macular edema (e.g., macular edema occurring after retinal vein occlusion, refraction and accommodation disorders, keratoconus, amblyopia, glaucoma, Stargardt disease, endophthalmitis, conjunctivitis, uveitis (e.g., posterior uveitis), retinal detachment, corneal ulcers, dacryocystitis, Duane retraction syndrome, optic neuritis, choroidal neovascularization, choroidal ischemia, or hypertensive retinopathy.

In some embodiments, the ocular disease or disorder is a retinal dystrophy (e.g., a Mendelian-heritable retinal dystrophy). In some embodiments, the retinal dystrophy is selected from the group consisting of Leber's congenital amaurosis (LCA), Stargardt Disease, pseudoxanthoma elasticum, rod cone dystrophy, exudative vitreoretinopathy, Joubert Syndrome, congenital stationary night blindness, type 1C (CSNB-1C), age-related macular degeneration, retinitis pigmentosa, stickler syndrome, microcephaly and choriorretinopathy, retinitis pigmentosa, CSNB 2, Usher syndrome, and Wagner syndrome.

In some instances, the methods provided herein are useful for treatment of symptoms of such ocular diseases or disorders, such as any of the above diseases or disorders, or ocular symptoms of broader disorders, such as hypotension, hypertension, infection, sarcoid, or sickle cell disease. In some embodiments, the disease is an acute ocular disease. In other embodiments, the disease is a chronic ocular disease.

In some embodiments, the individual to be treated is a human patient. In some embodiments, the individual is a pediatric human patient, e.g., a person aged 21 years or younger at the time of their diagnosis or treatment. In some embodiments, the pediatric human patient is aged 16 years or younger at the time of treatment. In other embodiments, the individual is aged 22 to 40 years at the time of treatment. In other embodiments, the individual is aged 41 to 60 years at the time of treatment. In other embodiments, the individual is aged 61 years or older at the time of treatment. In some instances, the individual is male. In other instances, the individual is female.

### Administration of Therapeutic Agents

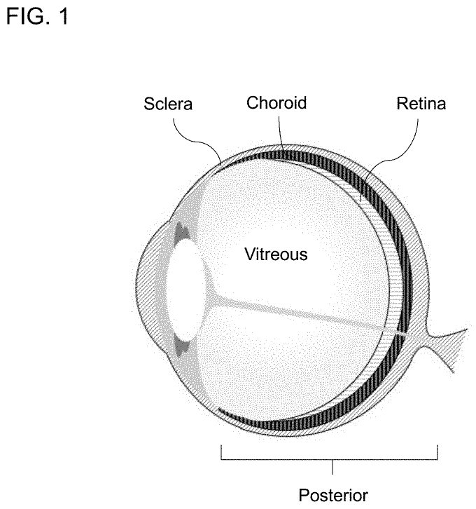

Provided herein are methods of administering therapeutic agents (e.g., nucleic acid vectors (e.g., any of the nucleic acid vectors described herein)), or pharmaceutical compositions thereof, to the eye as a means to deliver a therapeutic agent into a target retinal cell of an individual (e.g., a human patient). An anatomical illustration of the eye is shown in FIG. 1, for reference. In some instances, the nucleic acid vector is administered to the eye such that the nucleic acid vector enters the extracellular space of a posterior region of the eye (e.g., the retina (e.g., the macula)). Once the nucleic acid vector is in the posterior extracellular space upon administration (e.g., as a naked formulation, encapsulated in a nanoparticle or microparticle (e.g., a lipid nanoparticle or microparticle), or released from a nanoparticle or microparticle), it can subsequently be electrotransferred into the target retinal cell upon transmission of electrical energy reaching into the posterior of the eye (e.g., the retina (e.g., the macula)), e.g., though transmission of electrical energy from an electrode positioned in the vitreous chamber or subretinal space. In some instances, the nucleic acid vector is administered to the eye such that the nucleic acid vector enters the extracellular space of a posterior region of the eye (e.g., the posterior suprachoroid or the posterior choroid). Once the nucleic acid vector is in the posterior extracellular space upon administration, it can subsequently be electrotransferred into the target retinal cell upon transmission of electrical energy reaching into the posterior of the eye (e.g., the posterior suprachoroid or the posterior choroid), e.g., though transmission of electrical energy from an electrode positioned in the vitreous chamber or subretinal space.

In some embodiments, the nucleic acid vector is administered prior to a method described herein (e.g., prior to a method of transmitting an electrical field into a retinal tissue). For instance, a nucleic acid vector can be administered within 24 hours preceding transmission of an electric field (e.g., within 20 hours, within 18 hours, within 16 hours, within 14 hours, within 12 hours, within 10 hours, within 8 hours, within 6 hours, within 4 hours, within 3 hours, within 2 hours, within 90 minutes, within 60 minutes, within 45 minutes, within 30 minutes, within 20 minutes, within 15 minutes, within 10 minutes, within 5 minutes, within 4 minutes, within 3 minutes, within 2 minutes, within 1 minute, within 45 seconds, within 30 seconds, within 20 seconds, within 15 seconds, within 10 seconds, or within 5 seconds preceding transmission of an electric field). In some embodiments, the nucleic acid vector is administered after a method described herein (e.g., after a method of transmitting an electrical field into a retinal tissue), e.g., in instances in which the nucleic acid vector is released from a nanoparticle or microparticle over time. For instance, a nucleic acid vector can be administered within 24 hours after transmission of an electric field (e.g., within 20 hours, within 18 hours, within 16 hours, within 14 hours, within 12 hours, within 10 hours, within 8 hours, within 6 hours, within 4 hours, within 3 hours, within 2 hours, within 90 minutes, within 60 minutes, within 45 minutes, within 30 minutes, within 20 minutes, within 15 minutes, within 10 minutes, within 5 minutes, within 4 minutes, within 3 minutes, within 2 minutes, within 1 minute, within 45 seconds, within 30 seconds, within 20 seconds, within 15 seconds, within 10 seconds, or within 5 seconds after transmission of an electric field). In some embodiments, the nucleic acid vector is administered as part of a method described herein.

Any suitable means of ocular administration known in the art or described herein may be used as part of the methods provided herein. Methods of delivering a therapeutic agent to a target retinal cell include administering the nucleic acid vector to the eye by intraocular injection (e.g., injection to the posterior of the eye or the anterior of the eye, e.g., suprachoroidal injection, intravitreal injection, subretinal injection, periocular injection, sub-tenton injection, posterior juxtascleral injection, intracameral injection, subconjunctival injection, or retrobulbar injection) or intraocular implant. In some embodiments of any of the methods described herein, the administration of the nucleic acid vector is via an intraocular implant (e.g., a controlled release or depot implant, an intravitreal implant, a subconjunctival implant, or an episcleral implant). In other embodiments, the administration of the nucleic acid vector is not via an intraocular implant (e.g., a controlled release or depot implant, an intravitreal implant, a subconjunctival implant, or an episcleral implant). In some embodiments of any of the methods described herein, the administration of the nucleic acid vector is via iontophoresis (e.g., the method includes administration of the nucleic acid vector to the intraocular space by iontophoresis and subsequent delivery to the retina by transmitting a current through an electrode contacting an interior region of the eye, as described herein). In other embodiments, the administration of the nucleic acid vector does not involve iontophoresis.

In some instances, administration of the nucleic acid vector is non-surgical. For example, in some embodiments, administration of the nucleic acid vector does not utilize general anesthesia and/or does not involve retrobulbar anesthesia (i.e., retrobulbar block)). Additionally, or alternatively, administration of the nucleic acid vector does not involve injection using a needle larger than 28 gauge. Additionally, or alternatively, administration of the nucleic acid vector does not involve use of a guidance mechanism that is typically required for ocular drug delivery via shunt or cannula.

In some instances, administration of the nucleic acid vector is by injection (e.g., microneedle injection) into an outer tissue of the eye, e.g., the suprachoroidal space, sclera, cornea, corneal stroma, conjunctiva, subconjunctival space, or subretinal space. Alternatively, administration of the nucleic acid vector is by injection (e.g., microneedle injection) into a site proximal to the outer tissue, such as the trabecular meshwork, ciliary body, aqueous humor or vitreous humor.

Microneedles for injecting a nucleic acid vector to eye include hollow microneedles, which may include an elongated housing for holding the proximal end of the microneedle. Microneedles may further include a means for conducting a drug formulation therethrough. For example, the means may be a flexible or rigid conduit in fluid connection with the base or proximal end of the microneedle. The means may also include a pump or other devices for creating a pressure gradient for inducing fluid flow through the device. The conduit may in operable connection with a source of the drug formulation. The source may be any suitable container. In one embodiment, the source may be in the form of a conventional syringe. The source may be a disposable unit, dose container. In one embodiment, the microneedle has an effective length of about 50 μm to about 2000 μm. In another particular embodiment, the microneedle has an effective length of from about 150 μm to about 1500 μm, from about 300 μm to about 1250 μm, from about 500 μm to about 1250 μm, from about 500 μm to about 1500 μm, from about 600 μm to about 1000 μm, or from about 700 μm to about 1000 μm. In one embodiment, the effective length of the microneedle is about 600 μm, about 700 μm, about 800 μm or about 1000 μm. In various embodiments, the proximal portion of the microneedle has a maximum width or cross-sectional dimension of from about 50 μm to 600 μm, from about 50 μm to about 400 μm, from about 50 μm to about 500 μm, from about 100 μm to about 400 μm, from about 200 μm to about 600 μm, or from about 100 μm to about 250 μm, with an aperture diameter of about 5 μm to about 400 μm. In a particular embodiment, the proximal portion of the microneedle has a maximum width or cross-sectional dimension of about 600 μm. In various embodiments, the microneedle has a bevel height from 50 μm to 500 μm, 100 μm to 500 μm. 100 μm to 400 μm, 200 μm to 400 μm. or 300 μm to 500 μm.

The microneedle may have an aspect ratio (width:length) of about 1:1.5 to about 1:10. In one embodiment, the aspect ratio of the microneedle is about 1:3 to about 1:5. In another embodiment, the aspect ratio of the microneedle is about 1:4 to about 1:10.

In particular embodiments, the microneedle may be designed such that the tip portion of the microneedle is substantially the only portion of the microneedle inserted into the ocular tissue (i.e., the tip portion is greater than 75% of the total length of the microneedle, greater than 85% of the total length of the microneedle, or greater than about 95% of the total length of the microneedle). In other particular embodiments, the microneedle may be designed such that the tip portion is only a portion of the microneedle that is inserted into the ocular tissue and generally has a length that is less than about 75% of the total length of the microneedle, less than about 50% of the total length of the microneedle, or less than about 25% of the total length of the microneedle. For example, in one embodiment the microneedle has a total effective length between 500 μm and 1500 μm, wherein the tip portion has a length that is less than about 400 μm, less than about 300 μm, or less than about 200 μm.

In one embodiment, the height of the bevel from 100 μm to about 500 μm. In another embodiment, the height of the bevel is 500 μm or less, 450 μm or less, 400 μm or less, or 350 μm or less. In another embodiment, the height of the bevel is from 200 μm to 500 μm, from 100 μm to 700 μm, or from 200 μm to about 700 μm. In still other embodiments, the height of the bevel is from 500 μm to 900 μm, from 500 μm to 800 μm, or from 500 μm to 700 μm. In this manner, the arrangement of the bevel can be such that the distal edge is sufficiently sharp such as to pierce a target tissue and penetrate into the vitreous without (i) substantially causing the target tissue to elastically deform or (ii) damaging internal structures of the eye, e.g., the lens or retina.

Microneedles useful in the present methods can be made from different biocompatible materials, including metals, glasses, semi-conductor materials, ceramics, or polymers. Examples of suitable metals include pharmaceutical grade stainless steel, gold, titanium, nickel, iron, gold, tin, chromium, copper, platinum, and alloys thereof. Suitable polymers can be biodegradable or non-biodegradable. Examples of suitable biocompatible, biodegradable polymers include polylactides, polyglycolides, polylactide-co-glycolides (PLGA), polyanhydrides, polyorthoesters, polyetheresters, polycaprolactones, polyesteramides, poly(butyric acid), poly(valeric acid), polyurethanes and copolymers and blends thereof. Representative non-biodegradable polymers include various thermoplastics or other polymeric structural materials known in the fabrication of medical devices. Examples include nylons, polyesters, polycarbonates, polyacrylates, polymers of ethyiene-vinyi acetates and other acyl substituted cellulose acetates, non-degradable polyurethanes, polystyrenes, polyvinyl chloride, polyvinyl fluoride, poly(vinyl imidazole), chlorosulphonate poiyolefins, polyethylene oxide, blends and copolymers thereof. Biodegradable microneedles can provide an increased level of safety compared to nonbiodegradable ones, such that they are essentially harmless even if inadvertently broken off into the ocular tissue.

In particular instances, administration of the nucleic acid vector is by suprachoroidal injection, which can be accomplished in a minimally invasive, non-surgical manner. For instance, suprachoroidal injection can provide nucleic acid delivery over a larger tissue area and to less accessible target tissues in a single administration as compared to other types of administration (e.g., subretinal injection). Without wishing to be bound by theory, upon entering the suprachoroidal space, a pharmaceutical composition can flow circumferentially toward the retinochoroidal tissue, macula, and optic nerve in the posterior segment of the eye. In addition, a portion of the infused pharmaceutical composition may remain in the suprachoroidal space as a depot, or remain in tissue overlying the suprachoroidal space, for example the sclera, near the microneedle insertion site, serving as additional depot of the pharmaceutical composition that can subsequently diffuse into the suprachoroical space and into other adjacent posterior tissues.

Suprachoroidal injection can be performed using any suitable method known in the art or described herein. For example, in some instances, the nucleic acid vector is suprachoroidally administered through a microneedle (e.g., a hollow microneedle). In some instances, the nucleic acid vector is suprachoroidally administered through a microneedle array. Exemplary microneedles suitable for use in suprachoroidal administration of nucleic acid vectors described herein are described, e.g., in U.S. Patent Application No. 2017/0273827, which is incorporated herein by reference.

Suprachoroidal injection can be performed using methods known in the art. For example, a microneedle tip can be placed into the eye so that the drug formulation flows into the suprachoroidal space and to the posterior ocular tissues surrounding the suprachoroidal space. In one embodiment, insertion of the microneedle is in the sclera of the eye. In one embodiment, drug flow into the suprachoroidal space is achieved without contacting underlying tissues with the microneedle, such as choroid and retina tissues. In some embodiments, the one or more microneedles are inserted perpendicularly, or at an angle from 80° to 100°, into the eye, e.g., into the sclera, reaching the suprachoroidal space in a short penetration distance. Exemplary methods suitable for use in suprachoroidal administration of nucleic acid vectors described herein are described, e.g., in International Patent Publication No. WO 2014/074823, which is incorporated herein by reference.

In some embodiments, the device includes an array of two or more microneedles. For example, the device may include an array of from 2 to 1000 (e.g., from 2 to 100) microneedles. In one embodiment, a device includes between 1 and 50 microneedles. An array of microneedles may include a mixture of different microneedles. For instance, an array may include microneedles having various lengths, base portion diameters, tip portion shapes, spacings between microneedles, drug coatings, etc.

In embodiments wherein the microneedle device comprises an array of two or more microneedles, the angle at which a single microneedle extends from the base may be independent from the angle at which another microneedle in the array extends from the base.

In some instances, the present methods of delivering a therapeutic agent (e.g., nucleic acid vector (e.g., a non-viral DNA vector, e.g., a circular DNA vector that lacks a bacterial original of replication, a drug resistance gene, and/or a recombination site)) involve administration of the therapeutic agent intravitreally. Intravitreal administration can be conducted using any suitable method known in the art or described herein. For instance, contemplated herein are intravitreal injection methods involving the InVitria Injection Assistant (FCI Ophthalmics, Pembroke, MA), Rapid Access Vitreal Injection (RAVI) Gude (Katalyst Surgical, Chesterfield, MO), Doi-Umeatsu Intravitreal Injection Guide (Duckworth & Kent Ltd., England), Malosa Intravitreal Injection Guide (Beaver-Visitec International, Waltham, Mass.), or automated injection guides.

The present invention includes methods in which the nucleic acid vector is suprachoroidally administered through a device (e.g., a microinjector device) comprising a cannula and/or microneedle (e.g., any of the microneedles described above). Exemplary devices suitable for use in suprachoroidal administration of nucleic acid vectors described herein are described, e.g., in U.S. Pat. No. 10,722,396, U.S. Design Patent No. 750223S1, and Hancock et al., Expert Opinion on Drug Delivery 2021, DOI: 10.1080/17425247.2021.1867532, each of which is incorporated herein by reference.

In some instances, the suprachoroidal injection occurs within the pars plana, e.g., from 1-5 mm from the limbus. Microneedles for use in such injections can be designed to have a length that substantially matches the scleral thickness at the pars plana (e.g., from 400 μm to 600 μm, e.g., about 500 μm).

In some embodiments of any of the methods described herein involving suprachoroidal injection, the suprachoroidal injection is a bilateral suprachoroidal injection (e.g., divided into two injections). In other embodiments, the suprachoroidal injection is a 50onoliteral suprachoroidal injection (e.g., a single injection).

In some instances, methods of delivering a therapeutic agent to a target retinal cell include administering the nucleic acid vector systemically (e.g., intravenously or orally).

Any suitable dose of nucleic acid vector may be administered. For instance, in embodiments involving subretinal administration of naked nucleic acid vector, each eye may be injected with one or more blebs each having a volume from 20-500 μL (e.g., from 50-250 μL; e.g., 50-100 μL, 100-150 μL, 150-200 μL, or 200-250 μL; e.g., about 50 μL, about 75 μL, about 100 μL, about 150 μL, or about 200 μL), e.g., one bleb, two blebs, three blebs, four blebs, or more, per eye. In embodiments involving subretinal administration of naked nucleic acid vector, the injection volume (e.g., pharmaceutical composition) contains the nucleic acid vector at a concentration from 0.5 mg/mL to 5 mg/mL (e.g., from 1.0 mg/mL to 2.5 mg/mL; e.g., from 0.5 mg/mL to 1.0 mg/mL, from 1.0 mg/mL to 1.5 mg/mL, from 1.5 mg/mL to 2.0 mg/mL, from 2.0 mg/mL to 2.5 mg/mL, from 2.5 mg/mL to 3.0 mg/mL, from 3.0 mg/mL to 4.0 mg/mL, or from 4.0 mg/mL to 5.0 mg/mL; e.g., about 0.5 mg/mL, about 1.0 mg/mL, about 1.5 mg/mL, about 2.0 mg/mL, about 2.5 mg/mL, about 3.0 mg/mL, about 4.0 mg/mL, or about 5.0 mg/mL. In particular instances (e.g., wherein naked nucleic acid vector is administered subretinally), the injection volume (e.g., pharmaceutical composition) contains the nucleic acid vector at a concentration of 1.5 mg/mL. In some embodiments involving subretinal administration, naked nucleic acid vector is administered to each eye in an amount from 20 μg to 2.0 mg (e.g., from 100 μg to 1.0 mg, or from 200 μg to 500 μg; e.g., from 20 μg to 50 μg, from 50 μg to 100 μg, from 100 μg to 150 μg, from 150 μg to 200 μg, from 200 μg to 250 μg, from 250 μg to 300 μg, from 300 μg to 350 μg, from 350 μg to 400 μg, from 400 μg to 500 μg, from 500 μg to 750 μg, from 750 μg to 1.0 mg, from 1.0 mg to 1.5 mg, or from 1.5 mg to 2.0 mg; e.g., about 20 μg, about 25 μg, about 30 μg, about 40 μg, about 50 μg, about 60 μg, about 70 μg, about 75 μg, about 80 μg, about 90 μg about 100 μg, about 125 μg, about 150 μg, about 175 μg, about 200 μg, about 225 μg, about 250 μg, about 275 μg, about 300 μg, about 350 μg, about 400 μg, about 500 μg, about 600 μg, about 700 μg, about 800 μg, about 900 μg, about 1.0 mg, about 1.1. mg, about 1.2 mg, about 1.3 mg, about 1.4 mg, about 1.5 mg, about 1.6 mg, about 1.7 mg, about 1.8 mg, about 1.9 mg, or about 2.0 mg). In some embodiments involving subretinal administration, naked nucleic acid vector is administered to each eye in an amount from 10⁸ to 10¹⁵ vector copies (e.g., DNA vector molecules, e.g., circular DNA vector molecules) (e.g., from 10⁸ to 10⁹, from 10⁹ to 10¹⁰, from 10¹⁰ to 10¹¹, from 10¹¹ to 10¹², from 10¹² to 10¹³, from 10¹³ to 10¹⁴, or from 10¹⁴ to 10¹⁵ vector copies; e.g., about 1×10¹¹ vector copies, about 5×10¹¹ vector copies, about 1×10¹² vector copies, about 5×10¹² vector copies, about 1×10¹³ vector copies, about 2.5×10¹³ vector copies, or about 5×10¹³ vector copies). In particular embodiments, naked nucleic acid vector is administered subretinally (e.g., in two 75 μL-blebs per eye) at a total dose per eye of about 2.5×10¹³ vector copies. In other embodiments, naked nucleic acid vector is administered subretinally (e.g., in two 75 μL-blebs per eye) at a total dose per eye of about 5×10¹² vector copies. In other embodiments, naked nucleic acid vector is administered subretinally (e.g., in two 75 μL-blebs per eye) at a total dose per eye of about 5×10¹¹ vector copies.

### Transmission of Electric Fields

Methods of delivering therapeutic agents (e.g., nucleic acid vectors) to the eye include transmitting electrical energy into the tissue in which the target ocular cell resides. Such methods involve electrotransfer of the therapeutic agent from the extracellular space in the posterior of the eye (e.g., the suprachoroidal space, choroid, retina, or vitreous) into the target ocular cell (e.g., retinal cell). For example, in some instances in which an individual is being treated for a retinal disease or disorder, the method involves transmitting electrical energy into the retina to cause electrotransfer of a therapeutic agent (e.g., a nucleic acid vector) from the extracellular space of the retina into one or more retinal cell types (e.g., a photoreceptor and/or a retinal pigment epithelial cell).

In some aspects of the present invention, an electrode is positioned within the interior of the individual's eye, and an electric field is transmitted through the electrode into a target ocular tissue (e.g., retina at conditions suitable for electrotransfer of the therapeutic agent (e.g., nucleic acid vector) into the target cell (e.g., target retinal cell). An electric field (e.g., a pulsed electric field (PEF)) transmitted into a target ocular tissue can promote transfer of a nucleic acid vector (e.g., circular DNA vector) into a target ocular cell. Such electrotransfer can occur through any one of several mechanisms (and combinations thereof), including electrophoresis, electrokinetically driven drug uptake, and/or electroporation. Transmission of electric fields involve conditions suitable for such mechanisms. Suitable means of generating electric fields for electrotransfer of nucleic acids in mammalian tissue are known in the art, and any suitable means known in the art or described herein can be adapted for use as part of the present invention.

Various means of generating and transmitting an electric field into a tissue are contemplated herein as part of the present methods. Devices and systems having electrodes suitable for transmitting electric fields in mammalian tissues are commercially available and can be useful in the methods disclosed herein. In some instances, the electric field is transmitted through an electrode comprising a needle (e.g., a needle positioned within the vitreous humor or in the subretinal space). Suitable needle electrodes include CLINIPORATOR® electrodes marketed by IGEA® and needle electrodes marketed by AMBU®. Electrodes (e.g., needle electrodes) can be made from any suitable conductive material, such as metal or metal alloy, such as platinum, stainless steel, nickel, titanium, and combinations thereof, such as platinum/iridium alloy or nitinol.

In some embodiments, the electrode used as part of methods described herein is a substantially planar electrode, such as any of the substantially planar electrodes described in U.S. Patent Application Nos. 63/163,350, 63/167,296, and 63/293,297, the disclosures of which are hereby incorporated by reference in their entirety. In some embodiments, the electrode used as part of methods described herein is a substantially planar electrode as described herein (see, e.g., Devices section below). Such substantially planar electrodes are composed of a shape memory material (e.g., a shape memory alloy) that allows the structure of an elongate conductor (e.g., a wire electrode) to relax into a preformed, substantially planar electrode when unsheathed. The substantially planar electrode is approximately perpendicular to the longitudinal axis of the sheath and/or the proximal portion of the wire (e.g., the region that does not include the substantially planar electrode). One of skill in the art would appreciate that in some embodiments, the substantially planar electrode may not be perfectly planar. For example, in some embodiments, two of its perpendicular dimensions (e.g., Cartesian dimensions, such as, depth and width) are each at least twice its third perpendicular dimension (e.g., length). In some embodiments, a substantially planar electrode refers to an electrode in which two of its perpendicular dimensions are each at least 3 times, at least 4 times, at least 5 times, at least 6 times, at least 7 times, at least 8 times, at least 9 times, at least 10 times, at least 20 times, at least 30 times, at least 40 times, at least 50 times, at least 60 times, at least 70 times, at least 80 times, or at least 100 times, or more of its third perpendicular dimension. Thus, in some instances, a longitudinal dimension of the substantially planar electrode is less than 10% of a radial dimension of the substantially planar electrode (e.g., the outermost radial point). In some instances, a longitudinal dimension of the substantially planar electrode is less than 5% of its radial dimension (e.g., the outermost radial point).

In certain embodiments, the spatial configuration of the electrode is fabricated to optimize its conductive properties and/or exert a desired electric field on a target region of cells. Accordingly, as the eye includes a curvature, the shape of the electrode may also include a curvature (e.g., a convex shape), e.g., that matches or approximates the shape of the eye or a portion thereof (e.g., the retina).

In some examples, the elongate conductor is a wire, and the substantially planar electrode is the distal portion of the wire. In some instances, the distal tip of the wire (or a point along the wire within 5 mm (e.g., within 4 mm, within 3 mm, within 2 mm, within 1 mm, within 0.5 mm, or within 0.1 mm) of the distal tip of the wire) is at the outermost radial point of the substantially planar electrode. The distal portion of the wire may include a preformed right angle (or substantially a right angle, e.g., about 70°, 75°, 80°, 85°, 86°, 87°, 88°, 89°, 90°, 91°, 92°, 93°, 94°, 95°, 100°, 105°, or 110°) on a longitudinal plane, wherein the preformed right angle is between the substantially planar electrode and the proximal portion of the wire.

In some embodiments, the substantially planar electrode extends no further than 1 mm (e.g., no further than 0.9 mm, 0.8 mm, 0.7 mm, 0.6 mm, 0.5 mm, 0.4 mm, 0.3 mm, 0.2 mm, or 0.1 mm) distal or proximal to the preformed right angle. In some embodiments, the substantially planar electrode extends no further than 1 mm (e.g., no further than 0.9 mm, 0.8 mm, 0.7 mm, 0.6 mm, 0.5 mm, 0.4 mm, 0.3 mm, 0.2 mm, or 0.1 mm) distal to the preformed right angle. In some embodiments, the device includes nothing distal to the substantially planar electrode (e.g., the substantially planar electrode is free to contact the tissue surface).

In some embodiments, the substantially planar electrode is from about 2 mm to about 15 mm (e.g., about 2 mm, 3 mm, 4 mm, 5 mm, 6 mm, 7 mm, 8 mm, 9 mm, 10 mm, 11 mm, 12 mm, 13 mm, 14 mm, or 15 mm) in one or more dimensions (e.g., both dimensions) perpendicular to the longitudinal axis.

In some embodiments, the substantially planar electrode is substantially symmetrical about a longitudinal plane. In some embodiments, the substantially planar electrode is a spiral. For example, the spiral may include 1 to 5 (e.g., 1, 1.5, 2, 2.5, 3, 2.5, 4, 4.5, 5, 5.5, 6, 7, 8, or more) revolutions about the longitudinal axis. In some instances, the spiral has 2-5 revolutions about the longitudinal axis. In some instances, the spiral has 2 to 3 revolutions about the longitudinal axis. In particular embodiments, the spiral has 2 revolutions about the longitudinal axis. In some embodiments, the spiral has 3 revolutions about the longitudinal axis. Other suitable shapes include, for example, a loop, concentric loops, paddle, mesh, grid, or umbrella shape.

Substantially planar electrodes can be made wholly or partially from a shape memory material (e.g., shape memory alloy, e.g., NiTi) that can recover its original shape at the presence of a predetermined stimulus. For example, a shape memory material can relax into a preformed shape upon removal of a structural constraint. As described herein, a preformed shape memory wire (e.g., a substantially planar electrode) housed in a rigid sheath is straight until it is unsheathed, at which point the shape memory material relaxes into its preformed shape, such as a spiral. Shape memory materials are known in the art. In some embodiments, the shape memory material includes an alloy, such as NiTi, CuAlNi, or CuZnAl. The shape memory material may be ferrous. In some embodiments, the shape memory material is NiTi. NiTi is an alloy of nickel and titanium (nitinol). The nitinol may include, e.g., from about 40% to about 70% nickel (e.g., about 40%, 45%, 50%, 55%, 60%, 65%, or 70% nickel).

Electrodes (e.g., a substantially planar electrodes or a non-substantially planar electrodes (e.g., substantially axial wire electrodes)) for use in the present methods may be monopolar. In some embodiments involving electrotransfer using a monopolar electrode, a ground electrode is attached to the individual (e.g., attached to the skin of an individual) at a point other than the eye. In some embodiments, the ground electrode is a pad contacting the skin of the buttocks, leg, torso, neck (e.g., the posterior of the neck), or head (e.g., the posterior of the head) of the individual. In some embodiments, the monopolar electrode transmits electrical energy upon becoming positively charged. In some embodiments, the monopolar electrode transmits electrical energy upon becoming negatively charged.

Alternatively, electrodes may be bipolar (e.g., a substantially planar electrodes or a non-substantially planar electrodes may be bipolar (e.g., substantially axial wire electrodes may be bipolar)). In a bipolar embodiment, an auxiliary electrode may be in electrical communication with the primary electrode (e.g., substantially planar electrode or a non-substantially planar electrode (e.g., substantially axial wire electrode)). The auxiliary electrode may be proximal to the primary electrode (i.e., closer to the operator), e.g., part of, or connected to, a sheath housing a primary wire electrode. In some embodiments involving electrotransfer using a bipolar electrode, electrical energy (e.g., current) is transmitted upon application of a positive voltage to the primary electrode and a negative voltage to the auxiliary electrode. In some embodiments involving electrotransfer using a bipolar electrode, electrical energy (e.g., current) is transmitted upon application of a negative voltage to the primary electrode and a positive voltage to the auxiliary electrode.

In some instances, methods of the invention involve contacting an electrode (e.g., a substantially planar electrode or a non-substantially planar electrode (e.g., a substantially axial wire electrode)) to an interior region of the eye such that electrical energy transmitted from the electrode is sufficient to cause electrotransfer at the target tissue (e.g., the retina, e.g., the macula). Thus, methods of the invention may include positioning the electrode into electrical communication with the target tissue (e.g., retina, e.g., the macula). In particular instances, the interior region of the eye contacting the electrode includes the vitreous humor. For example, the electrode may be positioned wholly or partially within the vitreous humor upon transmission of the electric field. In instances in which the electrode is positioned within the vitreous humor (e.g., wholly within the vitreous humor), the electrode may be positioned in electrical communication with the interface of the vitreous humor with the retina (e.g., an interface at the macula).

In any of the aforementioned embodiments, the proximity of the electrode (e.g., a substantially planar electrode or the tip of a needle electrode) to the target cell results in a voltage (e.g., potential) at the target cell that is within 20% of the amplitude of the waveform of the applied energy (e.g., within 19%, within 18%, within 17%, within 16%, within 15%, within 14%, within 13%, within 12%, within 11%, within 10%, within 9%, within 8%, within 7% within 6%, within 5%, within 4%, within 3%, within 2%, or within 1% of the amplitude of the waveform of the applied energy).

It will be appreciated that a variety of suitable electrical parameters and algorithms thereof may be used. The voltage source may be configured to generate an electric field strength, e.g., at a target cell, from about 10 V/cm to about 1,500 V/cm (e.g., from about 10 V/cm to about 100 V/cm, e.g., about 10 V/cm, 20 V/cm, 30 V/cm, 40 V/cm, 50 V/cm, 60 V/cm, 70 V/cm, 80 V/cm, 90 V/cm, or 100 V/cm, e.g., from about 100 V/cm to about 1,000 V/cm, e.g., about 200 V/cm, 300 V/cm, 400 V/cm, 500 V/cm, 600 V/cm, 700 V/cm, 800 V/cm, 900 V/cm, or 1,000 V/cm, e.g., from about 1,000 V/cm to about 1,500 V/cm, e.g., about 1,110 V/cm, 1,200 V/cm, 1,300 V/cm, 1,400 V/cm, or 1,500 V/cm). In some embodiments, the voltage source is be configured to generate an electric field strength, e.g., at a target cell, from about 10 V/cm to about 1,000 V/cm (e.g., from about 10 V/cm to 500 V/cm or from about 500 V/cm to about 1,000 V/cm). In some embodiments, the field strength is from 50 V/cm to 300 V/cm. In some embodiments, the field strength is about 100 V/cm at the target cell.

In some embodiments, the voltage (e.g., potential) at the target cell is from 5 V to 100 V (e.g., from 10 V to 80V, from 15 V to 70 V, from 20 V to 60 V, or from 30 V to 50 V; e.g., about 10 V, about 15 V, about 20 V, about 25 V, about 30 V, about 35 V, about 40 V, about 45 V, about 50 V, about 55 V, about 60 V, about 65 V, about 70 V). In some embodiments, the voltage (e.g., potential) at the target cell is from 20 V to 60 V. In some embodiments, the voltage (e.g., potential) at the target cell is from 30 V to 50 V, e.g., about 35 V to 45 V. In any of the aforementioned embodiments, close proximity of the electrode to the target cell results in a voltage (e.g., potential) at the target cell that is within 20% of the amplitude of the waveform of the applied energy (e.g., within 19%, within 18%, within 17%, within 16%, within 15%, within 14%, within 13%, within 12%, within 11%, within 10%, within 9%, within 8%, within 7% within 6%, within 5%, within 4%, within 3%, within 2%, or within 1% of the amplitude of the waveform of the applied energy). For instance, a 40 V amplitude pulse from a monopolar intravitreal electrode positioned near the retina may result in a voltage (e.g., potential) of 35 V at a target retinal cell. It will be understood that waveform amplitudes required to achieve a given voltage at a target cell will depend on the electrode configuration (e.g., monopolar vs bipolar), electrode shape, distance between electrode and the target cell, and material properties (e.g., conductivity) of the tissue (e.g., vitreous and retina).

In some embodiments, the current resulting from the pulsed electric field is from 10 μA to 1 A (e.g., from 10 μA to 500 mA, from 10 μA to 200 mA, from 10 μA to 100 mA, from 10 μA to 50 mA, or from 10 μA to 25 mA; e.g., from 50 μA to 500 mA, from 100 μA to 200 mA, or from 1 mA to 100 mA; e.g., from 10 μA to 20 μA, from 20 μA to 30 μA, from 30 μA to 50 μA, from 50 μA to 100 μA, from 100 μA to 150 μA, from 150 μA to 200 μA, from 200 μA to 300 μA, from 300 μA to 400 μA, from 400 μA to 500 μA, from 500 μA to 600 μA, from 600 μA to 800 μA, from 800 μA to 1 mA, from 1 mA to 10 mA, from 10 mA to 20 mA, from 20 mA to 30 mA, from 30 mA to 40 mA, from 40 mA to 50 mA, from 50 mA to 60 mA, from 60 mA to 70 mA, from 70 mA to 80 mA, from 80 mA to 90 mA, from 90 mA to 100 mA, from 100 mA to 200 mA, from 200 mA to 300 mA, from 300 mA to 500 mA, or from 500 mA to 1 A; e.g., about 1 mA, about 5 mA about 10 mA, about 15 mA, about 20 mA, about 25 mA, about 30 mA, about 35 mA, about 40 mA, about 45 mA, about 50 mA, about 60 mA, about 70 mA, about 80 mA, about 90 mA, or about 100 mA).

In some embodiments, the electrode is positioned within about 10 mm (e.g., within 9 mm, 8 mm, 7 mm, 6 mm, 5 mm, 4.5 mm, 4 mm, 3.5 mm, 3 mm, 2.5 mm, 2 mm, 1.5 mm, 1 mm, or 0.5 mm, 0.45 mm, 0.40 mm, 0.35 mm, 0.30 mm, 0.25 mm, 0.20 mm, 0.15 mm, or 0.10 mm) of the retinal interface. The electrode may be from 0.1 to about 0.5 mm (e.g., about 0.15 mm, 0.2 mm, 0.25 mm, 0.30 mm, 0.35 mm, 0 40 mm, 0.45 mm, or 0.5 mm), or from about 0.5 mm to 5 mm (e.g., about 0.5 mm, 1 mm, 1.5 mm, 2 mm, 2.5 mm, 3 mm, 3.5 mm, 4 mm, 4.5 mm, or 5 mm) from the retinal interface upon transmission of the one or more pulses. In some embodiments, the electrode (e.g., substantially planar electrode) is within about 1 mm from the retinal interface upon transmission of the one or more pulses.

The target cell (e.g., the target retinal cell, which may be a retinal cell in the macula) may be within about 5 mm (e.g., 4.5 mm, 4 mm, 3.5 mm, 3 mm, 2.5 mm, 2 mm, 1.5 mm, 1 mm, or 0.5 mm) from the retinal interface (e.g., at the macula). For example, the target cell may be from about 0.01 mm to about 1 mm (e.g., from about 0.01 mm to about 0.1 mm, e.g., about 0.02 mm, 0.03 mm, 0.04 mm, 0.05 mm, 0.06 mm, 0.07 mm, 0.08 mm, 0.09 mm, or 0.1 mm, e.g., from about 0.1 mm to about 1 mm, e.g., about 0.2 mm, 0.3 mm, 0.4 mm, 0.5 mm, 0.6 mm, 0.7 mm, 0.8 mm, 0.9 mm, or 1 mm) from the retinal interface.

It will be appreciated that a variety of suitable electrical parameters and algorithms thereof may be used. The voltage source may be configured to generate an electric field strength, e.g., at a target cell (e.g., a retinal cell), from about 10 V/cm to about 1,500 V/cm (e.g., from about 10 V/cm to about 100 V/cm, e.g., about 10 V/cm, 20 V/cm, 30 V/cm, 40 V/cm, 50 V/cm, 60 V/cm, 70 V/cm, 80 V/cm, 90 V/cm, or 100 V/cm, e.g., from about 100 V/cm to about 1,000 V/cm, e.g., about 200 V/cm, 300 V/cm, 400 V/cm, 500 V/cm, 600 V/cm, 700 V/cm, 800 V/cm, 900 V/cm, or 1,000 V/cm, e.g., from about 1,000 V/cm to about 1,500 V/cm, e.g., about 1,110 V/cm, 1,200 V/cm, 1,300 V/cm, 1,400 V/cm, or 1,500 V/cm). In some embodiments, the voltage source is be configured to generate an electric field strength, e.g., at a target cell (e.g., a retinal cell), from about 10 V/cm to about 1,000 V/cm (e.g., from about 10 V/cm to 500 V/cm or from about 500 V/cm to about 1,000 V/cm). In some embodiments, the field strength is from 50 V/cm to 300 V/cm. In some embodiments, the field strength is about 100 V/cm at the target cell (e.g., the target retinal cell).

In some embodiments, the total number of pulses of electrical energy are delivered within 1-60 seconds (e.g., within 1-5 seconds, 5-10 seconds, 10-15 seconds, 15-20 seconds, 20-30 seconds, 30-40 seconds, 40-50 seconds, or 50-60 seconds). In some embodiments, the total number of pulses of electrical energy are delivered within 1-20 seconds. For example, the total number of pulses of electrical energy may be delivered within 1-5 seconds, 5-10 seconds, 10-15 seconds, or 15-20 seconds, e.g., 1, 2, 3, 4, 5, 6, 7, 8, 9, 10, 11, 12, 13, 14, 15, 16, 17, 18, 19, or 20 seconds. The pulses of electrical energy may be, e.g., square waveforms. The pulses of electrical energy may have an amplitude from 5 V to 500 V. For example, the pulses of electrical energy may have an amplitude of about 5 V, 10 V, 15 V, 20 V, 25 V, 30 V, 35V, 40 V, 45 V, 50 V, 60 V, 70 V, 80 V, 90 V, 100 V, 125 V, 150 V, 175 V, 200 V, 225V, 250 V, 275 V, 300 V, 325 V, 350 V, 375 V, 400 V, 425 V, 450 V, 475 V, or 500 V. In some embodiments, the pulses of electrical energy have an amplitude of about 5-250 V (e.g., about 20 V). Any of the aforementioned voltages can be the tops of square-waveforms, peaks in sinusoidal waveforms, peaks in sawtooth waveforms, root mean square (RMS) voltages of sinusoidal waveforms, or RMS voltages of sawtooth waveforms.

In some embodiments, about 1-12 pulses (e.g., 1, 2, 3, 4, 5, 6, 7, 8, 9, 10, 11, or 12 pulses) of electrical energy are transmitted during use. In some embodiments, about 4-12 pulses of electrical energy are transmitted during use.

In some embodiments, each of the pulses of electrical energy is from about 10 ms to about 200 ms. For example, each of the pulses of electrical energy may be about 10 ms, 20 ms, 30 ms, 40 ms, 50 ms, 60 ms, 70 ms, 80 ms, 90 ms, 100 ms, 110 ms, 120 ms, 130 ms, 140 ms, 150 ms, 160 ms, 170 ms, 180 ms, 190 ms, or 200 ms. In some embodiments, each of the pulses of electrical energy is from about 50 ms. In some embodiments, each of the pulses of electrical energy is less than 10 ms. For example, each of the pulses of electrical energy may be from about 10 μs to about 10 ms, e.g., from about 10 μs to about 100 μs, e.g., about 20 μs, 30 μs, 40 μs, 50 μs, 60 μs, 70 μs, 80 μs, 90 μs, or 100 μs, e.g., from about 100 μs to about 1 ms, e.g., about 200 μs, 300 μs, 400 μs, 500 μs, 600 μs, 700 μs, 800 μs, 900 μs, or 1 ms, e.g., from about 1 ms to about 10 ms, e.g., about 2 ms, 3 ms, 4 ms, 5 ms, 6 ms, 7 ms, 8 ms, 9 ms, or 10 ms.

An electric field suitable for electrotransfer can be transmitted to a target ocular cell at or near the time of administration of a therapeutic agent (e.g., nucleic acid vector (e.g., circular DNA vector)) or pharmaceutical composition thereof (e.g., as part of the same procedure). For example, the present invention includes methods in which an electric field is transmitted within one hour of administration of the therapeutic agent (e.g., nucleic acid vector (e.g., circular DNA vector)) or pharmaceutical composition thereof (e.g., within 55 minutes, within 50 minutes, within 45 minutes, within 40 minutes, within 35 minutes, within 30 minutes, within 25 minutes, within 20 minutes, within 15 minutes, within 10 minutes, within 5 minutes, within 4 minutes, within 3 minutes, within 2 minutes, within 90 seconds, within 60 seconds, within 45 seconds, with 30 seconds, within 20 seconds, within 15 seconds, within 10 seconds, within 9 seconds, within 8 seconds, within 7 seconds, within 6 seconds, within 5 seconds, within 4 seconds, within 3 seconds, within 2 seconds, or within 1 second) of administration of the nucleic acid vector (e.g., circular DNA vector) or pharmaceutical composition thereof (e.g., simultaneously with administration of the nucleic acid vector (e.g., circular DNA vector) or pharmaceutical composition thereof or after administration but within any of the aforementioned durations). In some embodiments, an electric field is transmitted within 24 hours of administration of the nucleic acid vector (e.g., circular DNA vector) or pharmaceutical composition thereof (e.g., within 22 hours, within 20 hours, within 18 hours, within 16 hours, within 14 hours, within 12 hours, within 10 hours, within 8 hours, within 6 hours, within 5 hours, within 4 hours, within 3 hours, within 2 hours, within 90 minutes, within 60 minutes, within 45 minutes, within 30 minutes, within 20 minutes, within 15 minutes, within 10 minutes, within 8 minutes, within 6 minutes, within 5 minutes, within 4 minutes, within 3 minutes, or within 2 minutes) of administration of the nucleic acid vector (e.g., circular DNA vector) or pharmaceutical composition thereof. In some embodiments, an electric field is transmitted within 7 days of administration of the nucleic acid vector (e.g., circular DNA vector) or pharmaceutical composition thereof (e.g., within 6 days, within 5 days, within 4 days, within 3 days, or within 2 days) of administration of the nucleic acid vector (e.g., circular DNA vector) or pharmaceutical composition thereof.

An electric field suitable for electrotransfer can be transmitted at or near the site of administration (e.g., injection) of the therapeutic agent (e.g., nucleic acid vector (e.g., a non-viral DNA vector, e.g., a circular DNA vector that lacks a bacterial original of replication, a drug resistance gene, and/or a recombination site)) or pharmaceutical composition thereof. For instance, in some embodiments, the therapeutic agent (e.g., nucleic acid vector (e.g., a non-viral DNA vector, e.g., a circular DNA vector that lacks a bacterial original of replication, a drug resistance gene, and/or a recombination site)) or pharmaceutical composition thereof is administered intravitreally, and the electrode is positioned at or near the site of intravitreal administration for transmission of the electric field (e.g., within 10 mm, within 8 mm, within 6 mm, within 5 mm, within 4 mm, within 3 mm, within 2 mm, or within 1 mm of the site of intravitreal administration). In other embodiments, the therapeutic agent is administered (e.g., injected) subretinally, and the electrode is positioned at or near the site of subretinal administration for transmission of the electric field (e.g., within 10 mm, within 8 mm, within 6 mm, within 5 mm, within 4 mm, within 3 mm, within 2 mm, or within 1 mm of the site of subretinal administration). In other embodiments, the therapeutic agent (e.g., nucleic acid vector (e.g., a non-viral DNA vector, e.g., a circular DNA vector that lacks a bacterial original of replication, a drug resistance gene, and/or a recombination site)) or pharmaceutical composition thereof is administered suprachoroidally, and the electrode is positioned at or near the site of suprachoroidal administration for transmission of the electric field (e.g., within 10 mm, within 8 mm, within 6 mm, within 5 mm, within 4 mm, within 3 mm, within 2 mm, or within 1 mm of the site of suprachoroidal administration).

In some instances, the therapeutic agent (e.g., nucleic acid vector (e.g., a non-viral DNA vector, e.g., a circular DNA vector that lacks a bacterial original of replication, a drug resistance gene, and/or a recombination site)) or pharmaceutical composition thereof is administered at a location that is exposed to the electric field (or will be exposed to the electric field, in the event of subsequent electric field transmission). In some embodiments, the therapeutic agent (e.g., nucleic acid vector (e.g., circular DNA vector)) or pharmaceutical composition thereof is delivered at a location that is exposed to (or will be exposed to) a voltage that is 1% or more of the maximum tissue voltage (e.g., at least 5% of the maximum tissue voltage, at least 10% of the maximum tissue voltage, at least 20% of the maximum tissue voltage, at least 30% of the maximum tissue voltage, at least 40% of the maximum tissue voltage, at least 50% of the maximum tissue voltage, at least 60% of the maximum tissue voltage, at least 70% of the maximum tissue voltage, at least 80% of the maximum tissue voltage, or at least 90% of the maximum tissue voltage, e.g., from 1% to 10% of the maximum tissue voltage, from 10% to 20% of the maximum tissue voltage, from 20% to 30% of the maximum tissue voltage, from 30% to 40% of the maximum tissue voltage, from 40% to 50% of the maximum tissue voltage, from 50% to 60% of the maximum tissue voltage, from 60% to 70% of the maximum tissue voltage, from 70% to 80% of the maximum tissue voltage, from 80% to 90% of the maximum tissue voltage, from 90% to 95% of the maximum tissue voltage, or from 95% to 100% of the maximum tissue voltage).

Alternatively, the site of administration can be in a region of tissue away from the electric field. For example, administration of the therapeutic agent (e.g., nucleic acid vector (e.g., a non-viral DNA vector, e.g., a circular DNA vector that lacks a bacterial original of replication, a drug resistance gene, and/or a recombination site)) or pharmaceutical composition thereof may be systemic (e.g., intravenous), while the electric field is transmitted in the eye (e.g., in the vitreous humor or in the subretinal space). In any of the methods described herein involving electrotransfer (e.g., by PEF), a paralytic may be administered according to standard procedures, which can help reduce the risk and/or severity of muscle contractions upon transmission of electrical energy.

### Treatment Characterization

The level or concentration of an ocular protein (e.g., retinal protein) expressed from a nucleic acid vector described herein may be an expression level, presence, absence, truncation, or alteration of the administered vector. It can be advantageous to correlate the level with one or more clinical phenotypes or with an assay for a human disease biomarker. The assay may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Therapeutic genes delivered by the nucleic acid vectors described herein may be quantified using methods such as, but not limited to, ultraviolet visible spectroscopy (UVNis). The quantified polynucleotide may be analyzed in order to determine if the polynucleotide may be of proper size, check that no degradation of the polynucleotide has occurred. Degradation of the polynucleotide may be checked by methods such as, but not limited to, agarose gel electrophoresis, HPLC based purification methods such as, but not limited to, strong anion exchange HPLC, weak anion exchange HPLC, reverse phase HPLC (RP-HPLC), hydrophobic interaction HPLC (HIC-HPLC), liquid chromatography-mass spectrometry (LCMS), capillary electrophoresis (CE), and capillary gel electrophoresis (CGE).

Efficacy of treatment can be monitored, assessed, and/or quantified using any suitable methods known in the art or provided herein. For example, an individual treated for a retinal disease or disorder may be monitored periodically to assess progression of retinal degeneration, e.g., by testing visual acuity and visual field using standard tests. Additionally, or alternatively, optical coherence tomography (OCT) (e.g., spectral domain OCT (SD-OCT)) can be conducted to assess changes in retinal structure. In some instances, an individual treated by the methods described herein exhibits improvement or no further degradation in retinal structure assessed by imaging endpoints, such as fundus autofluorescence (FAF) and/or SD-OCT.

Safety and tolerability of treatment can be monitored, assessed, and/or quantified using any suitable methods known in the art or provided herein. For instance, an individual treated for a retinal disease or disorder may be monitored periodically to assess cataract formation, intra-ocular inflammation, or retina damage such as RPE hypopigmentation. In some embodiments, an individual treated according to the methods described herein exhibits no cataract formation, no intraocular inflammation up to 2 months post-treatment (or less than grade 2 intraocular inflammation up to 2 months post-treatment), and/or minimal retina/RPE damage (e.g., RPE hypopigmentation).

In some instances, an individual is treated with nucleic acid vector and electrotransfer according to any of the embodiments described herein only once in their lifetime (e.g., treatment of the disease or disorder is sustained for several years (e.g., three to five years, five to ten years, ten to fifteen years, or at least 15 years). Alternatively, an individual may be treated exactly twice in their lifetime. Additionally, or alternatively, an individual may be treated once every 2-3 years, every 3-5 years, or every 5-10 years.

### IV. Devices

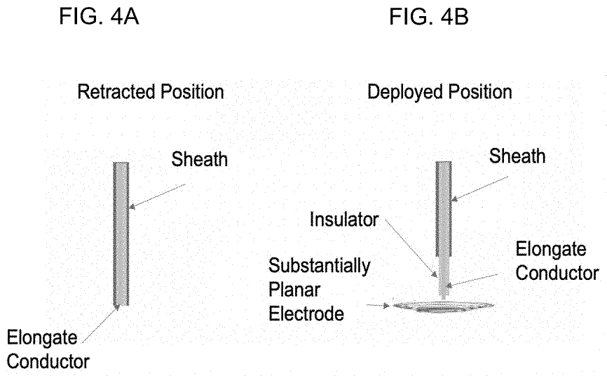

The devices described herein include a sheath having a proximal end, a distal end, and a longitudinal axis therebetween. The device includes an elongate conductor having a proximal portion within the sheath and a distal portion. In some embodiments (e.g., embodiments involving a planar electrode), the elongate conductor is composed of a preformed shape memory material and is retractable within the sheath from a proximal position, where the conductor is in a retracted position (FIG. 4A), to a distal position, where the elongate conductor is deployed (FIG. fB). In the proximal position, the distal portion of the elongate conductor is substantially straight. In the distal position, the distal portion of the elongate conductor extends beyond the distal end of the sheath, and the shape memory material of the distal portion of the elongate conductor is relaxed radially. Upon radial relaxation, the elongate conductor forms a substantially planar electrode that is approximately perpendicular to the longitudinal axis of the sheath.

Also featured are devices that include a sheath having a proximal end, a distal end, and a longitudinal axis therebetween. The device further includes an elongate conductor having a proximal portion within the sheath and a distal portion, wherein the elongate conductor includes a preformed shape memory material and is retractable within the sheath from a proximal position to a distal position. In the proximal position, the distal portion of the elongate conductor is substantially straight. In the distal position, the distal portion of the elongate conductor extends beyond the distal end of the sheath, and the shape memory material of the distal portion of the elongate conductor is relaxed radially to form an electrode that is disposed at a preformed angle (e.g., from about 10° to about 170°, e.g., from about 20° to about 160°, e.g., from about 30° to about 150°, e.g., from about 45° to about 135°, e.g., from about 60° to about 120°, e.g., from about 70° to about 110′, e.g., from about 80′ to about 100°, e.g., from about 85° to about 95°, e.g., about 10°, 20°, 30°, 30°, 45°, 50°, 55°, 60°, 65°, 70°, 71°, 72°, 73°, 74°, 75°, 76°, 77°, 78°, 79°, 80°, 81°, 82°, 83°, 84°, 85°, 86°, 87°, 88°, 89°, 90°, 91°, 92°, 93°, 94°, 95°, 96°, 97°, 98°, 99°, 100°, 101°, 102°, 103°, 104°, 105°, 106°, 107°, 108°, 109°, 110°, 115°, 120°, 125°, 130°, 135°, 140°, 145°, 150°, 160°, or 170°) relative to the longitudinal axis of the sheath. In some embodiments, the electrode is a substantially planar electrode. In some embodiments, the preformed angle is substantially a right angle.

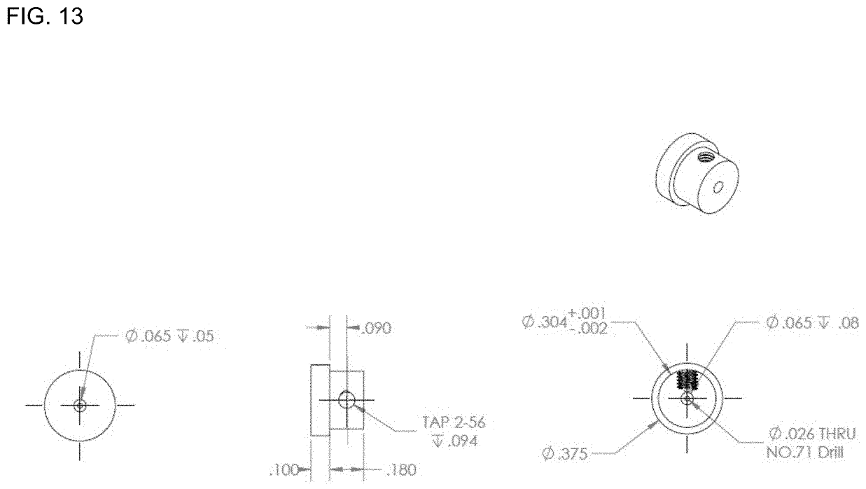

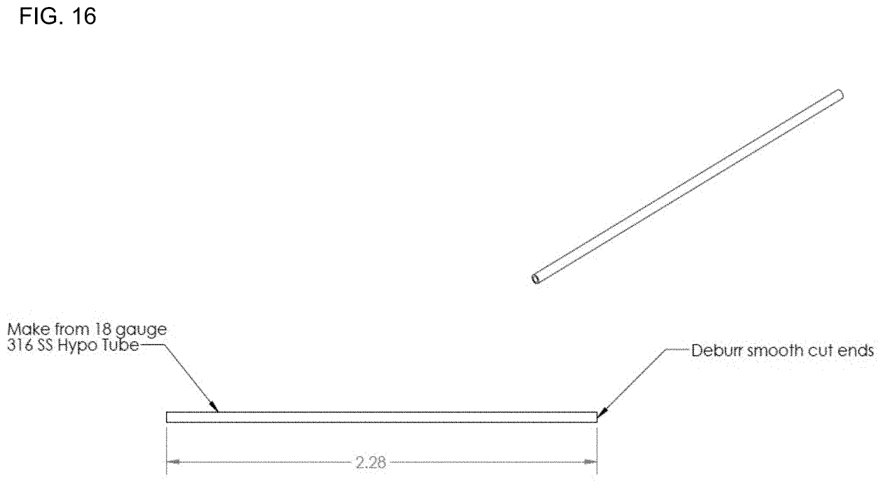

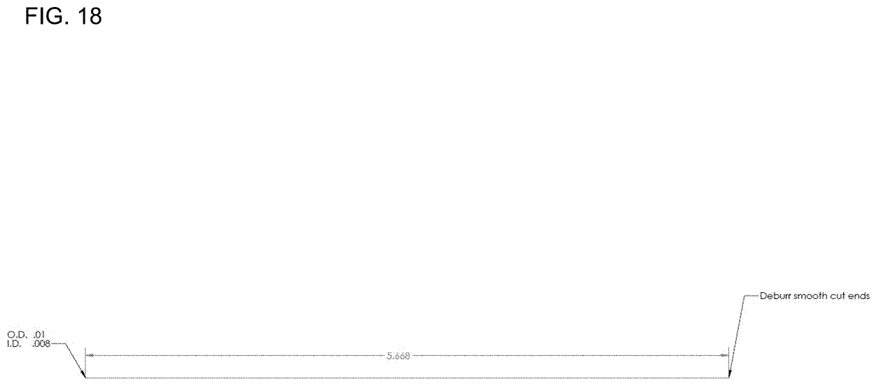

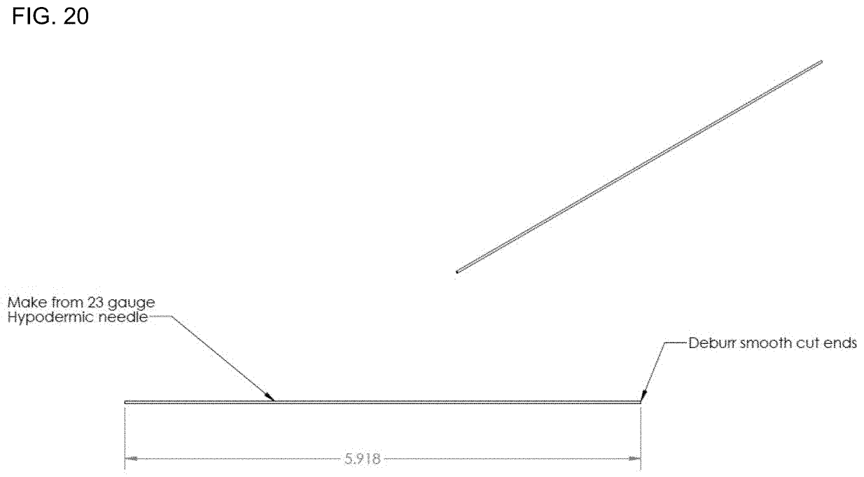

The components of such a device described herein are shown, for example, in FIGS. 13-20. While these figures show various dimensions and parameters for each component, one of skill in the art would understand that these dimension and parameters are exemplary and can be modified within the scope of the invention.

### Sheath

The device includes a sheath through which an elongate conductor is deployed. The sheath is hollow and may contain any suitable size or shape to allow the conductor to deploy and retract therewithin. The sheath may be substantially straight or curved. The sheath may be rigid or flexible, e.g., to provide facile manipulation to reach a target region. The sheath has substantial rigidity to allow the elongate conductor to remain constrained therewithin, e.g., when in the retracted position.

The sheath may have a length from about 1 mm to about 100 cm, e.g., from about 1 cm to about 75 cm, from about 2 cm to about 50 cm, from about 5 cm to about 40 cm, from about 10 cm to about 35 cm, or from about 15 cm to about 20 cm. For example, the sheath may have a length of from about 0.1 mm to about 1 mm, e.g., about 0.2 mm, 0.3 mm, 0.4 mm, 0.5 mm, 0.6 mm, 0.7 mm, 0.8 mm, 0.9 mm, or 1 mm, e.g., about 1 mm to about 10 mm, e.g., about 2 mm, 3 mm, 4 mm, 5 mm, 6 mm, 7 mm, 8 mm, 9 mm, 10 mm, e.g., from about 10 mm to about 100 mm, e.g., about 20 mm, 30 mm, 40 mm, 50 mm, 60 mm, 70 mm, 80 mm, 90 mm, or 100 mm, e.g., from about 1 cm to about 10 cm, e.g., about 2 cm, 3 cm, 4 cm, 5 cm, 6 cm, 7 cm, 8 cm, 9 cm, or 10 cm, e.g., from about 10 cm to about 100 cm, e.g., about 15 cm, 20 cm, 25 cm, 30 cm, 35 cm, 40 cm, 45 cm, 50 cm, 55 cm, 60 cm, 65 cm, 70 cm, 75 cm, 80 cm, 85 cm, 90 cm, 95 cm, or 100 cm.

The sheath may be a substantially hollow tube or other suitable shape and contains an inner and outer diameter that is dependent on the thickness of the sheath. A cross-section of the sheath may be substantially circular or elliptical. The cross-section of the sheath may be polygonal (e.g., triangle or square etc.). In some embodiments, the outer cross-section is a first shape (e.g., a circle, ellipse, or polygon, e.g., triangle or square) and the inner cross-section is a second shape (e.g., a circle, ellipse, or polygon, e.g., triangle or square). The inner diameter of the sheath may be from about 0.01 mm to about 5 mm, e.g., from about 0.1 mm to about 1 mm, e.g., from about 0.2 mm to about 0.5 mm, e.g., from about 0.2 mm to about 0.3 mm. For example, the inner diameter of the sheath may be from 0.01 mm to about 5 mm, e.g., from about 0.01 mm to about 0.1 mm, e.g., about 0.02 mm, 0.03 mm, 0.04 mm, 0.05 mm, 0.06 mm, 0.07 mm, 0.08 mm, 0.09 mm, or 0.1 mm, e.g., from about 0.1 mm to about 1 mm, e.g., about 0.2 mm, 0.3 mm, 0.4 mm, 0.5 mm, 0.6 mm, 0.7 mm, 0.8 mm, 0.9 mm, or 1 mm, e.g., from about 1 mm to about 5 mm, e.g., about 2 mm, 2.5 mm, 3 mm, 3.5 mm, 4 mm, 4.5 mm, or 5 mm.

The outer diameter of the sheath, which is greater than the inner diameter, may be from about 0.01 mm to about 5 mm, e.g., from about 0.1 mm to about 1 mm, e.g., from about 0.2 mm to about 0.5 mm, e.g., from about 0.2 mm to about 0.3 mm. For example, the outer diameter of the sheath may be from 0.01 mm to about 5 mm, e.g., from about 0.01 mm to about 0.1 mm, e.g., about 0.02 mm, 0.03 mm, 0.04 mm, 0.05 mm, 0.06 mm, 0.07 mm, 0.08 mm, 0.09 mm, or 0.1 mm, e.g., from about 0.1 mm to about 1 mm, e.g., about 0.2 mm, 0.3 mm, 0.4 mm, 0.5 mm, 0.6 mm, 0.7 mm, 0.8 mm, 0.9 mm, or 1 mm, e.g., from about 1 mm to about 5 mm, e.g., about 2 mm, 2.5 mm, 3 mm, 3.5 mm, 4 mm, 4.5 mmm, or 5 mm.

The thickness of the sheath may be from about 0.01 mm to about 1 mm, e.g., from about 0.01 mm to about 0.1 mm, e.g., about 0.02 mm, 0.03 mm, 0.04 mm, 0.05 mm, 0.06 mm, 0.07 mm, 0.08 mm, 0.09 mm, or 0.1 mm, e.g., from about 0.1 mm to about 1 mm, e.g., about 0.2 mm, 0.3 mm, 0.4 mm, 0.5 mm, 0.6 mm, 0.7 mm, 0.8 mm, 0.9 mm, or 1 mm. The thickness of the sheath may be substantially uniform throughout or may have different thicknesses in different portions or regions of the sheath.

The sheath may be composed of a conductive material, such as a metal or metal alloy. Suitable sheath materials include, for example, stainless steel, titanium, a polymer, such as PEEK (e.g., that is machined, molded, or extruded) or polyimide, a composite, such as a woven polymer, e.g., with epoxy, or a ceramic. In some embodiments, the sheath is made of stainless steel. In some embodiments, the sheath is composed of nitinol. In some embodiments, the sheath is composed of stainless steel and contains a polymer tip, e.g., to facilitate retraction of the electrode wire.

The distal end of the sheath is configured to contact an eye such that the electrode can access a region in suitable proximity with (e.g., in electrical communication with) a desired target cell (e.g., in the vitreous humor near the surface of the retina). Accordingly, the distal end of the sheath may include a sharp feature, such as a pointed tip, to pierce the eye. The tip may be beveled (e.g., standard bevel, short bevel, or true short bevel). The distal end of the sheath may contain a needle (e.g., a hypodermic needle). The needle may be any suitable gauge or thickness to allow the elongate conductor to pass therethrough and/or match the thickness of the sheath, e.g., if desired. The gauge of the needle may be, e.g., from about 7 to about 33 (e.g., about 10 to 30, e.g., 12 to 28, e.g., 15-28, e.g., 20-28, e.g., 20-25, e.g., 7, 8, 9, 10, 11, 12, 13, 14, 15, 16, 17, 18, 19, 20, 21, 22, 23, 24, 25, 26, 27, 28, 29, 30, 31, 32, or 33. In some embodiments, the needle is 19 gauge. In some embodiments the needle is 23 gauge. In some embodiments, the needle is 25 gauge. In some embodiments, the needle is 30 gauge.

In some embodiments, the device includes a second sheath. The second sheath may be configured to be surrounded by the first sheath or a portion thereof. For example, the second sheath may have a diameter that is less than the diameter of the first sheath. In some embodiments, the second sheath is connected to the elongate conductor, e.g., at the proximal end of the elongate conductor. In some embodiments, the second sheath is connected to an actuator (e.g., slider) as described herein.

In some embodiments, the device (e.g., a device having a planar electrode, or a device having a non-planar, needle electrode) includes a sheath connected to the handle and a sheath (e.g., second sheath) connected to the slider (FIG. 13C). The elongate conductor may be within the sheath connected to the slider. In some embodiments, the sheath connected to the slider nests with the sheath connected to the handle. The sheath connected to the slider may be configured to be surrounded by the sheath connected to the handle or a portion thereof. For example, the sheath connected to the slider may have a diameter that is less than the diameter of the sheath connected to the handle. Alternatively, the sheath connected to the slider may surround the sheath connected to the handle or a portion thereof. For example, the sheath connected to the slider may have a diameter that is greater than the diameter of the sheath connected to the handle. In some embodiments, the sheath connected to the slider is connected to the elongate conductor, e.g., at the proximal end of the elongate conductor.

The inner diameter of the second sheath may be from about 0.01 mm to about 5 mm, e.g., from about 0.1 mm to about 1 mm, e.g., from about 0.2 mm to about 0.5 mm, e.g., from about 0.2 mm to about 0.3 mm. For example, the inner diameter of the second sheath may be from 0.01 mm to about 5 mm, e.g., from about 0.01 mm to about 0.1 mm, e.g., about 0.02 mm, 0.03 mm, 0.04 mm, 0.05 mm, 0.06 mm, 0.07 mm, 0.08 mm, 0.09 mm, or 0.1 mm, e.g., from about 0.1 mm to about 1 mm, e.g., about 0.2 mm, 0.3 mm, 0.4 mm, 0.5 mm, 0.6 mm, 0.7 mm, 0.8 mm, 0.9 mm, or 1 mm, e.g., from about 1 mm to about 5 mm, e.g., about 2 mm, 2.5 mm, 3 mm, 3.5 mm, 4 mm, 4.5 mm, or 5 mm.

The outer diameter of the second sheath, which is greater than the inner diameter, may be from about 0.01 mm to about 5 mm, e.g., from about 0.1 mm to about 1 mm, e.g., from about 0.2 mm to about 0.5 mm, e.g., from about 0.2 mm to about 0.3 mm. For example, the outer diameter of the second sheath may be from 0.01 mm to about 5 mm, e.g., from about 0.01 mm to about 0.1 mm, e.g., about 0.02 mm, 0.03 mm, 0.04 mm, 0.05 mm, 0.06 mm, 0.07 mm, 0.08 mm, 0.09 mm, or 0.1 mm, e.g., from about 0.1 mm to about 1 mm, e.g., about 0.2 mm, 0.3 mm, 0.4 mm, 0.5 mm, 0.6 mm, 0.7 mm, 0.8 mm, 0.9 mm, or 1 mm, e.g., from about 1 mm to about 5 mm, e.g., about 2 mm, 2.5 mm, 3 mm, 3.5 mm, 4 mm, 4.5 mmm, or 5 mm.

The thickness of the second sheath may be from about 0.01 mm to about 1 mm, e.g., from about 0.01 mm to about 0.1 mm, e.g., about 0.02 mm, 0.03 mm, 0.04 mm, 0.05 mm, 0.06 mm, 0.07 mm, 0.08 mm, 0.09 mm, or 0.1 mm, e.g., from about 0.1 mm to about 1 mm, e.g., about 0.2 mm, 0.3 mm, 0.4 mm, 0.5 mm, 0.6 mm, 0.7 mm, 0.8 mm, 0.9 mm, or 1 mm. The thickness of the second sheath may be substantially uniform throughout or may have different thicknesses in different portions or regions of the second sheath.

The second sheath may be or contain a needle (e.g., a hypodermic needle). The needle may be any suitable gauge or thickness to allow the first sheath and/or the elongate conductor to pass therethrough and/or match the thickness of the sheath, e.g., if desired. The gauge of the needle may be, e.g., from about 7 to about 33 (e.g., about 10 to 30, e.g., 12 to 28, e.g., 15-28, e.g., 20-28, e.g., 20-25, e.g., 7, 8, 9, 10, 11, 12, 13, 14, 15, 16, 17, 18, 19, 20, 21, 22, 23, 24, 25, 26, 27, 28, 29, 30, 31, 32, or 33. In some embodiments, the needle is 19 gauge. In some embodiments the needle is 23 gauge. In some embodiments, the needle is 25 gauge. In some embodiments, the needle is 30 gauge.

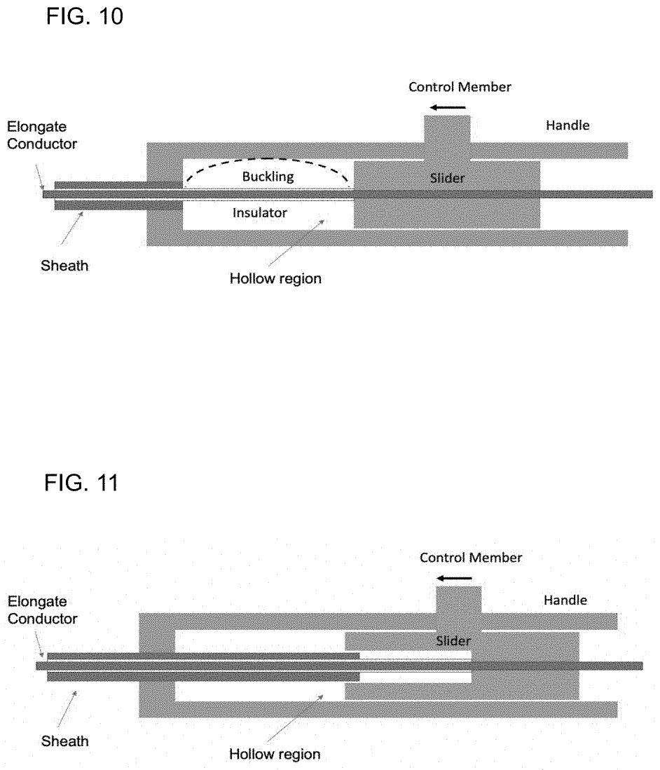

An embodiment with two sheaths (e.g., of a device having a planar electrode or a device having a non-planar electrode) may be particularly advantageous to prevent buckling of the elongate conductor, e.g., within the first sheath. For example, when the elongate conductor is substantially straight and within the sheath, the contact force between the conductor and the sheath is greater than the force to buckle the elongate conductor when pushed (FIG. 10). Therefore, the elongate conductor may buckle, and the distal end of the elongate conductor containing the substantially planar electrode cannot be properly deployed through the sheath. A second sheath may allow more efficient deployment of the substantially planar electrode without buckling of the elongate conductor. In particular embodiments, connecting the second sheath directly to the elongate conductor and/or the slider may prevent buckling.

In another embodiment, extending or disposing the distal end of the first sheath and/or the second sheath into the handle may also prevent buckling (FIGS. 10, 11, and 12A).

In some embodiments, the sheath (e.g., first sheath and/or second sheath) contains a coating on the inside and/or outside of the sheath. The coating may be employed to reduce friction, e.g., between sliding parts, such as the elongate conductor within the sheath and/or a second sheath (if used) and the first sheath.

### Elongate Conductor

The elongate conductor is disposed within the sheath and may be deployed from therewithin. The conductor may have a length of from about 1 mm to about 100 cm, e.g., from about 1 cm to about 75 cm, from about 2 cm to about 50 cm, from about 5 cm to about 40 cm, from about 10 cm to about 35 cm, or from about 15 cm to about 20 cm. For example, the conductor may have a length of from about 1 mm to about 10 mm, e.g., about 2 mm, 3 mm, 4 mm, 5 mm, 6 mm, 7 mm, 8 mm, 9 mm, 10 mm, e.g., from about 10 mm to about 100 mm, e.g., about 20 mm, 30 mm, 40 mm, 50 mm, 60 mm, 70 mm, 80 mm, 90 mm, or 100 mm, e.g., from about 1 cm to about 10 cm, e.g., about 2 cm, 3 cm, 4 cm, 5 cm, 6 cm, 7 cm, 8 cm, 9 cm, or 10 cm, e.g., from about 10 cm to about 100 cm, e.g., about 15 cm, 20 cm, 25 cm, 30 cm, 35 cm, 40 cm, 45 cm, 50 cm, 55 cm, 60 cm, 65 cm, 70 cm, 75 cm, 80 cm, 85 cm, 90 cm, 95 cm, or 100 cm.

The elongate conductor may be a substantially cylindrical (e.g., a cylindrical wire). A cross-section of the conductor may be substantially circular or elliptical. A cross-section of the conductor may be a polygon, e.g., a triangle, square, or the like. The diameter of the conductor may be from about 0.01 mm to about 5 mm, e.g., from about 0.1 mm to about 1 mm, e.g., from about 0.2 mm to about 0.5 mm, e.g., from about 0.2 mm to about 0.3 mm. For example, the diameter of the sheath may be from 0.01 mm to about 5 mm, e.g., from about 0.01 mm to about 0.1 mm, e.g., about 0.02 mm, 0.03 mm, 0.04 mm, 0.05 mm, 0.06 mm, 0.07 mm, 0.08 mm, 0.09 mm, or 0.1 mm, e.g., from about 0.1 mm to about 1 mm, e.g., about 0.2 mm, 0.3 mm, 0.4 mm, 0.5 mm, 0.6 mm, 0.7 mm, 0.8 mm, 0.9 mm, or 1 mm, e.g., from about 1 mm to about 5 mm, e.g., about 2 mm, 2.5 mm, 3 mm, 3.5 mm, 4 mm, 4.5 mmm, or 5 mm. In some embodiments, the diameter of the conductor is about 0.2 mm. The diameter of the conductor may be substantially uniform throughout or may have different diameter in different portions or regions of the conductor.

In some embodiments, the device incudes a plurality of elongate conductors, e.g., bundled together within the sheath. In an embodiment, the device includes two elongate conductors, and a cross-section of each conductor is substantially semicircular, or half an ellipse.

The diameter of the conductor may be from about 50% to about 99% of the inner diameter of the sheath. For example, the diameter may be from about 55% to about 95%, about 60% to about 90%, about 65% to about 85%, 70% to about 80%, or about 75%. The diameter of the conductor may be, e.g., 50%, 55%, 60%, 65%, 70%, 75%, 80%, 85%, 90%, 95%, 96%, 97%, 98%, or 99% of the inner diameter of the sheath.

The conductor may be composed of any suitable conductive material known in the art, such as a metal or metal alloy. In some instances, the conductor is composed of the same material as the sheath. In other instances, the conductor is a different material than the sheath. Suitable conductive materials useful for the conductor include, for example, platinum, platinum/iridium alloy, stainless steel, nickel, and titanium. In some embodiments, the conductor is made of an alloy of nickel and titanium alloy (e.g., nitinol). The nitinol may include, e.g., from about 40% to about 70% nickel (e.g., about 40% to about 45%, about 45% to about 50%, about 50% to about 55%, about 55% to about 60%, about 60% to about 65%, or about 65% to about 70%, e.g., about 40%, 45%, 50%, 55%, 60%, 65%, or 70% nickel).

### Substantially Planar Electrode

In some embodiments, the elongate conductor or a portion thereof (e.g., the distal portion) contains a substantially planar electrode. The substantially planar electrode is composed of a shape memory material (e.g., a shape memory alloy). A shape memory material allows the structure of the elongate conductor to relax into a preformed shape upon removal of a constraint (e.g., a structural element). For example, a preformed shape memory wire housed in a rigid sheath is constrained until it is unsheathed, at which point the shape memory material relaxes into its preformed shape (e.g., a substantially planar electrode) as is shown in FIGS. 4-6. In some embodiments, an actuator is used to deploy the substantially planar electrode (see, e.g., FIGS. 10 and 11).

In the devices described herein, the preformed shape may be a substantially planar electrode that is approximately perpendicular to the longitudinal axis of the sheath and/or the proximal portion of the elongate conductor (e.g., the region that does not include the substantially planar electrode). One of skill in the art would appreciate that in some embodiments, the substantially planar electrode may not be perfectly planar. For example, in some embodiments, two of its perpendicular dimensions (e.g., Cartesian dimensions, such as, depth and width) are each at least twice its third perpendicular dimension (e.g., length). In some embodiments, a substantially planar electrode refers to an electrode in which two of its perpendicular dimensions are each at least 3 times, at least 4 times, at least 5 times, at least 6 times, at least 7 times, at least 8 times, at least 9 times, at least 10 times, at least 20 times, at least 30 times, at least 40 times, at least 50 times, at least 60 times, at least 70 times, at least 80 times, or at least 100 times, or more of its third perpendicular dimension. Thus, in some instances, a longitudinal dimension of the substantially planar electrode is less than 10% of a radial dimension of the substantially planar electrode (e.g., the outermost radial point). In some instances, a longitudinal dimension of the substantially planar electrode is less than 5% of its radial dimension (e.g., the outermost radial point).

In certain embodiments, the spatial configuration of the electrode is fabricated to optimize its conductive properties and/or exert a desired electric field on a target region of cells. Accordingly, as the eye includes a curvature, the shape of the electrode may also include a curvature (e.g., a convex shape), e.g., that matches or approximates the shape of the eye or a portion thereof (e.g., the retina).

In some examples, the elongate conductor is a wire, and the substantially planar electrode is the distal portion of the wire. In some instances, the distal tip of the wire (or a point along the wire within 5 mm (e.g., within 4 mm, within 3 mm, within 2 mm, within 1 mm, within 0.5 mm, or within 0.1 mm) of the distal tip of the wire) is at the outermost radial point of the substantially planar electrode. The distal portion of the wire may include a preformed right angle (or substantially a right angle, e.g., about 70°, 75°, 80°, 85°, 86°, 87°, 88°, 89°, 90°, 91°, 92°, 93°, 94°, 95°, 100°, 105°, or 110°); or 110°); or a preformed angle of from about 45° to about 135° (e.g., about 45°, about 50°, about 55°, about 60°, about 65°, about 115°, about 120°, about 125°, about 130°, or about 135°) on a longitudinal plane, wherein the preformed angle (e.g., preformed right angle) is between the substantially planar electrode and the proximal portion of the wire.

In some embodiments, the shape memory material of the distal portion of the elongate conductor is relaxed radially to form an electrode that is disposed at a preformed angle (e.g., from about 10° to about 170°, e.g., from about 20° to about 160°, e.g., from about 30° to about 150°, e.g., from about 45° to about 135°, e.g., from about 60° to about 120°, e.g., from about 70° to about 110°, e.g., from about 80° to about 100°, e.g., from about 85° to about 95°, e.g., about 10°, 20°, 30°, 45°, 50°, 55°, 60°, 65°, 70°, 71°, 72°, 73°, 74°, 75°, 76°, 77°, 78°, 79°, 80°, 81°, 82°, 83°, 84°, 85°, 86°, 87°, 88°, 89°, 90°, 91°, 92°, 93°, 94°, 95°, 96°, 97°, 98°, 99°, 100°, 101°, 102°, 103°, 104°, 105°, 106°, 107°, 108°, 109°, 110°, 115°, 120°, 125°, 130°, 135°, 140°, 145°, 150°, 160°, or 170°) relative to the longitudinal axis of the sheath. In some embodiments, the electrode is a substantially planar electrode.

In some embodiments, the substantially planar electrode extends no further than 1 mm (e.g., no further than 0.9 mm, 0.8 mm, 0.7 mm, 0.6 mm, 0.5 mm, 0.4 mm, 0.3 mm, 0.2 mm, or 0.1 mm) distal or proximal to the preformed angle (e.g., preformed right angle). In some embodiments, the substantially planar electrode extends no further than 1 mm (e.g., no further than 0.9 mm, 0.8 mm, 0.7 mm, 0.6 mm, 0.5 mm, 0.4 mm, 0.3 mm, 0.2 mm, or 0.1 mm) distal to the preformed angle (e.g., preformed right angle). In some embodiments, the device includes nothing distal to the substantially planar electrode (e.g., the substantially planar electrode is free to contact the tissue surface).

In some embodiments, the substantially planar electrode is from about 2 mm to about 15 mm (e.g., about 2 mm, 3 mm, 4 mm, 5 mm, 6 mm, 7 mm, 8 mm, 9 mm, 10 mm, 11 mm, 12 mm, 13 mm, 14 mm, or 15 mm) in one or more dimensions (e.g., both dimensions), e.g., perpendicular to, or at a preformed angle relative to, the longitudinal axis.

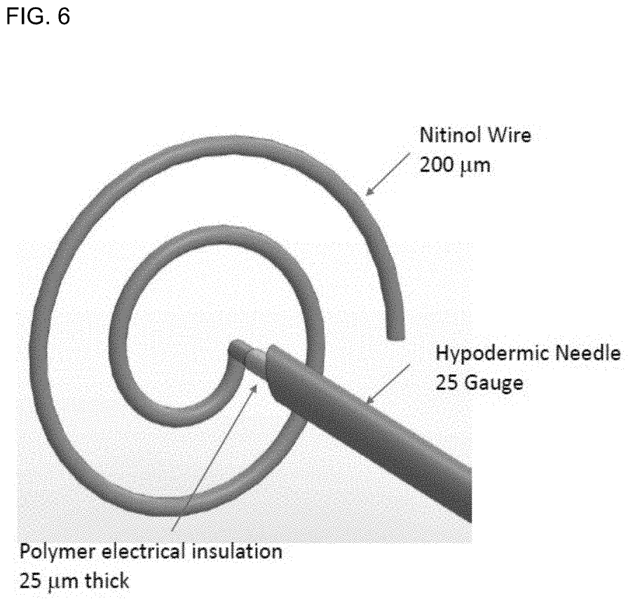

In some embodiments, the substantially planar electrode is substantially symmetrical about a longitudinal plane. In some embodiments, the substantially planar electrode is a spiral (FIG. 6). For example, the spiral may include 1 to 5 (e.g., 1, 1.5, 2, 2.5, 3, 2.5, 4, 4.5, 5, 5.5, 6, 7, 8, or more) revolutions about the longitudinal axis. In some instances, the spiral has 2-5 revolutions about the longitudinal axis. In some instances, the spiral has 2 to 3 revolutions about the longitudinal axis. In particular embodiments, the spiral has 2 revolutions about the longitudinal axis. In some embodiments, the spiral has 3 revolutions about the longitudinal axis. Other suitable shapes include, for example, a loop, concentric loops, paddle, mesh, grid, or umbrella shape. In some embodiments, the spiral consists of 3 revolutions about the longitudinal axis. In some embodiments, the spiral consists of 2 revolutions about the longitudinal axis (FIG. 6).

The substantially planar electrode can be made wholly or partially from a shape memory material (e.g., shape memory alloy, e.g., NiTi) that can recover its original shape at the presence of a predetermined stimulus. For example, a shape memory material can relax into a preformed shape upon removal of a structural constraint. As described herein, a preformed shape memory wire (e.g., a substantially planar electrode) housed in a rigid sheath is straight until it is unsheathed, at which point the shape memory material relaxes into its preformed shape, such as a spiral. Shape memory materials are known in the art. In some embodiments, the shape memory material includes an alloy, such as NiTi, CuAlNi, or CuZnAl. The shape memory material may be ferrous. In some embodiments, the shape memory material is NiTi. NiTi is an alloy of nickel and titanium (nitinol). The nitinol may include, e.g., from about 40% to about 70% nickel (e.g., about 40%, 45%, 50%, 55%, 60%, 65%, or 70% nickel).

### Insulator

The device may include an insulator disposed between the elongate conductor and the sheath. The insulator may be positioned between the proximal portion of the elongate conductor and the sheath. The insulator prevents an electrical contact between the sheath and the elongate conductor. The insulator may be made of any suitable material, such as glass, porcelain, or a polymeric (e.g., compositive polymeric) material. In some embodiments, the insulator is composed of polyimide or polyether ether ketone (PEEK). In some embodiments, the insulator is composed of polyvinylidene fluoride (PVDF), low-density polyethylene (LDPE), a blend of polyolefin and ethylene acrylic acid copolymer, high-density polyethylene (HDPE), fluorinated ethylene propylene (FEP), polyvinyl chloride (PVC), Parylene C, or a combination thereof. The insulation material may be deposited on the electrode surface or made, e.g., via heat-shrink tubing. The insulator may have a thickness of from about 1 μm to about 100 μm, e.g., from about 5 μm to about 90 μm, from about 10 μm to about 80, from about 10 μm to about 50 μm, or from about 20 μm to about 30 μm, e.g., about 25 μm. For example, the insulator may have a thickness of about 1 μm to about 10 μm, e.g., about 1 μm, 2 μm, 3 μm, 4 μm, 5 μm, 6 μm, 7 μm, 8 μm, 9 μm, or 10 μm, e.g., from about 10 μm to about 100 μm, e.g., about 15 μm, 20 μm, 25 μm, 30 μm, 35 μm, 40 μm, 45 μm, or 50 μm, 55 μm, 60 μm, 65 μm, 70 μm, 75 μm, 80 μm, 85 μm, 90 μm, 95 μm, or 100 μm.

In some embodiments, the device further includes an adhesive, glue, or epoxy disposed between the elongate conductor and the insulator.

### Handle

In some embodiments, the device described includes a handle. In certain embodiments, the proximal portion of the device includes a handle, e.g., for facile manipulation. The handle may be disposed on the sheath. The handle may be disposed, e.g., on the proximal portion of the elongate conductor. In some embodiments, the device includes a handle to manipulate the sheath and a handle of the proximate portion of the elongate conductor, e.g., to manipulate the conductor within the sheath.

The handle may have a proximal end and a distal end (FIGS. 10, 11, and 15). In some embodiments, the proximal end of the sheath is connected to the handle (e.g., connected to and disposed within the handle). In some embodiments, a distal portion of the handle includes a hollow region between an inner surface of the handle and the elongate conductor therewithin. The proximal end of the sheath may extend at least into the hollow region within the handle. In some embodiments, the proximal end of the sheath extends at least 1 mm, 2 mm, 3 mm, 4 mm, 5 mm, 6 mm, 7 mm, 8 mm, 9 mm, 10 mm, 15 mm, 20 mm, 25 mm, 30 mm, 35 mm, 40 mm, 45 mm, 50 mm, 55 mm, 60 mm, 65 mm, 70 mm, 80 mm, 85 mm, 90 mm, 95 mm, 100 mm, 110 mm, 120 mm, 130 mm, 140 mm, 150 mm, 160 mm, 170 mm, 180 mm, 190 mm, 200 mm, or more into the hollow region within the handle (FIG. 11).

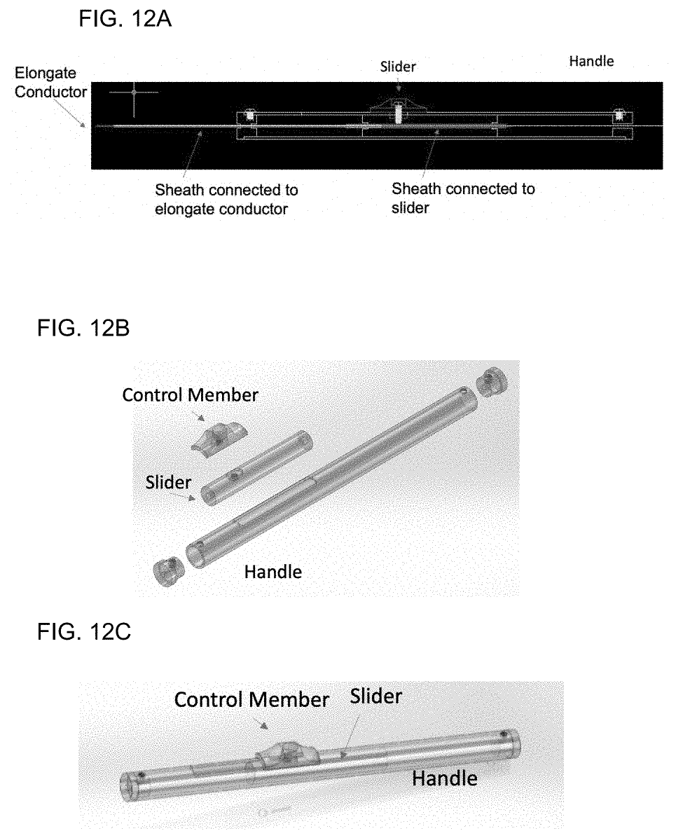

In some embodiments, the handle is cylindrical (FIGS. 12A-12C). In some embodiments, the handle further includes a cap on the distal and/or proximal end of the handle (FIGS. 12B, 13, and 14). For example, the handle may include a cap on each of the distal and proximal ends, e.g., to close off a hollow portion of the handle.

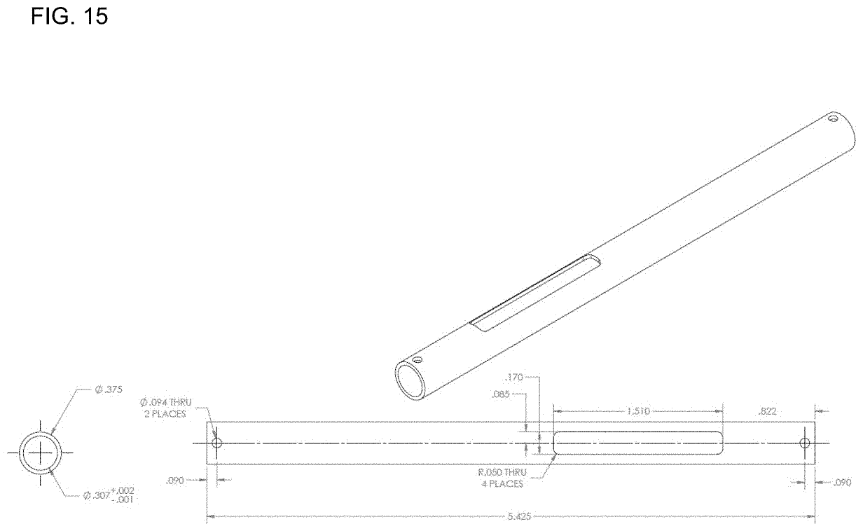

In some embodiments, the handle may have a length of from about 3 inches to about 10 inches, e.g., from about 3 inches to about 9 inches, e.g., about 3 inches, 4 inches, 5 inches, 6 inches, 7 inches, 8 inches, 9 inches, or 10 inches. In some embodiment, the length of the handle is from about 5 inches to about 6 inches, e.g., about 5.5 inches, e.g., about 5.425 inches (FIG. 15)

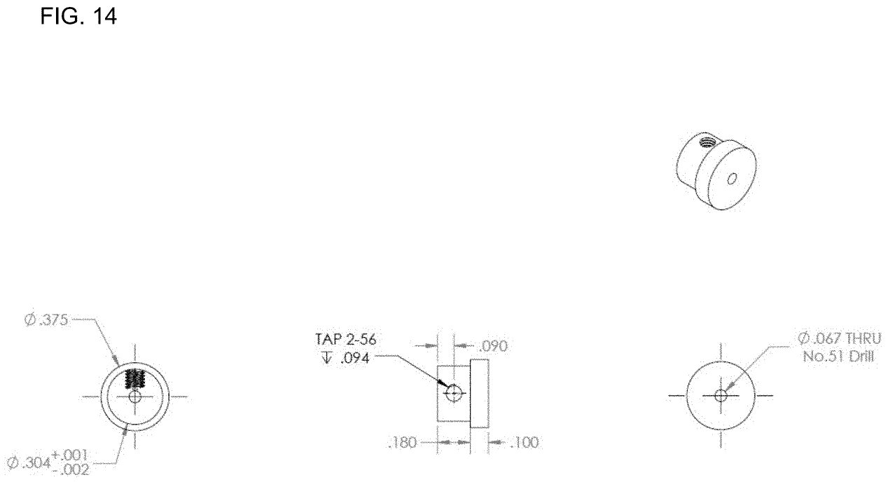

In some embodiments, the cap that fits within the distal and/or proximal end of the handle has a length of from about 0.1 inch to about 1.0 inch, e.g., about 0.1 inch, 0.2 inch, 0.3 inch, 0.4 inch, 0.5 inch, 0.6 inch, 0.7 inch, 0.8 inch, 0.9 inch, or 1.0 inch. In some embodiments, the length of the cap is from about 0.2 inch to about 0.3 inch, e.g., about 0.28 inch (FIGS. 13 and 14).

Actuator

The devices described herein may further include an actuator (e.g., a slider). The actuator (e.g., slider) may be configured to slide the elongate conductor between the proximal position and the distal position, e.g., between its relaxed and sheathed positions. The actuator may be a manual actuator. Alternatively, the actuator may be an electronically controlled actuator. In some embodiments, the actuator is a piezoelectric actuator.

In some embodiments, the actuator is operably coupled to the elongate conductor. In some embodiments, the actuator is present on a handle of the device.

In some embodiments, the actuator is a slider. The slider has a proximal end and a distal end and is attached (e.g., directly or indirectly) to the elongate conductor (see, e.g., FIGS. 10-12 and 27). The slider may be configured to retract the elongate conductor from the distal position to the proximal position along the longitudinal axis of the sheath. In some embodiments, the slider includes a proximal position and a distal position. In the proximal position, the proximal end of the sheath is disposed within or extends at least to the distal end of the slider. In the distal position, the proximal end of the sheath is disposed within or extends to between the proximal end and the distal end of the slider. In some embodiments, the slider is hollow, and the elongate conductor is disposed within or extends through the entire slider.

In some embodiments, the slider is configured to stop upon reaching the distal position and/or the proximal position. In some embodiments, the slider is disposed in the distal position and the distal portion of the elongate conductor extends beyond the distal end of the sheath. The shape memory material of the distal portion of the elongate conductor may be relaxed radially to form a substantially planar electrode at the preformed angle relative to the longitudinal axis of the sheath.

In some embodiments, the slider is disposed in the proximal position and the distal portion of the elongate conductor is substantially straight. In some embodiments, the slider further includes a control member disposed on an exterior of the handle. The control member may include a protrusion, knob, or other feature for facile control or ergonomic design of the slider. The control member and the slider may be integral. Alternatively, the control member and the slider may be non-integral, e.g., separate parts.

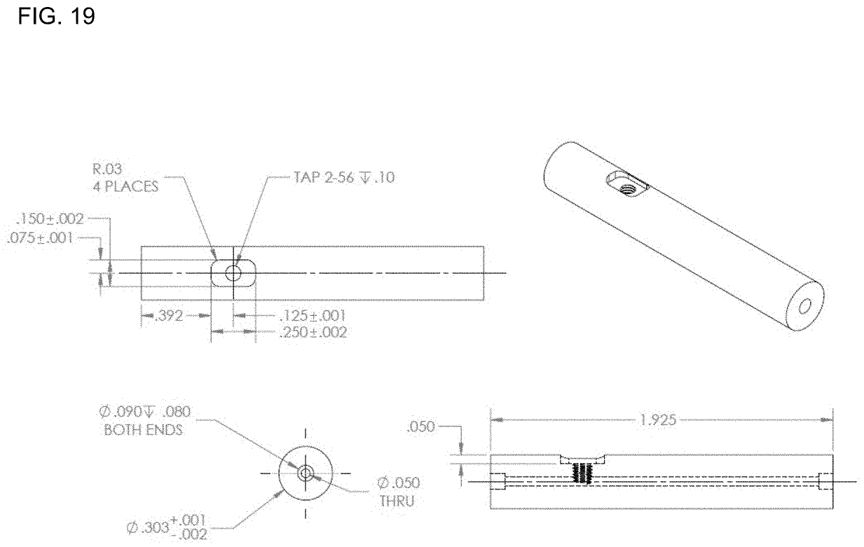

In some embodiments, the length of the slider is from about 0.5 inch to about 5.0 inches, e.g., from about 0.5 inch to about 3.5 inches, e.g., from about 1.0 inch to about 2.5 inches, e.g., about 2.0 inches, e.g., about 1.925 inches (FIG. 19).

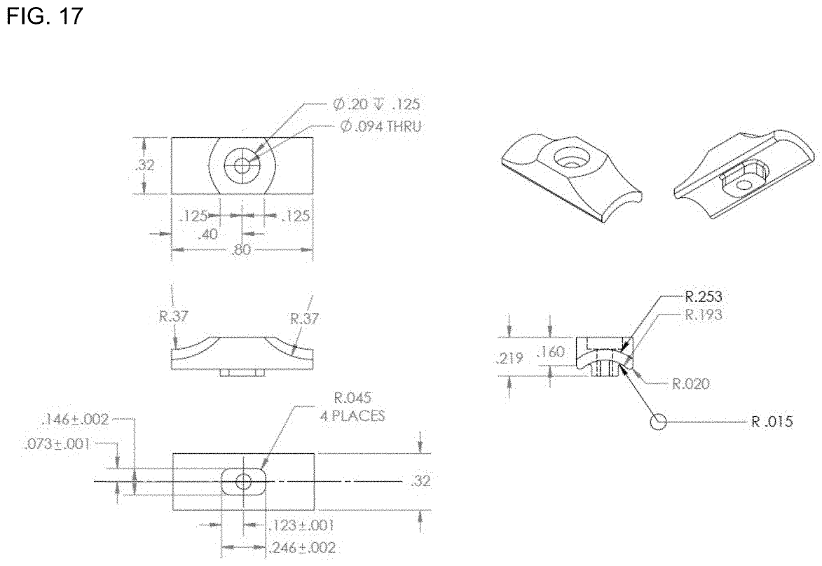

In some embodiments, the length of the control member is from about 0.1 inches to about 2.0 inches, e.g., about 0.2 inch, 0.3 inch, 0.4 inch, 0.5 inch, 0.6 inch, 0.7 inch, 0.8 inch, 0.9 inch, 1.0 inch, 1.1 inches, 1.2 inches, 1.3 inches, 1.4 inches, 1.5 inches, 1.6 inches, 1.7 inches, 1.8 inches, 1.9 inches, or 2.0 inches, e.g., about 0.5 inch to about 1.0 inch, e.g., about 0.8 inch (FIG. 17).

### Additional Elements

The device described herein may be monopolar. Alternatively, the device may be bipolar. In a bipolar embodiment, the device further includes an auxiliary electrode in electrical communication with the substantially planar electrode. The auxiliary electrode may be part of, or connected to, the sheath.

The device may further include a voltage source. The device may further include a waveform controller. In some embodiments, the proximal portion of the elongate conductor is connected to the voltage source and/or the waveform controller.

The device may be configured for use with an endoscope or bronchoscope. For example, the device may be positioned at a distal end of the endoscope of bronchoscope and may be deployed, e.g., upon insertion into a subject.

### V. Methods of Device Use

The invention features a method of using any of the devices described herein. In some instances, the invention provides a method of delivering a therapeutic agent into a target cell of an individual using a device as described herein. The method includes inserting the sheath or needle through an external tissue surface of the individual and sliding the elongate conductor to the distal position to allow the preformed shape memory material to relax radially, thereby forming the substantially planar electrode within the tissue. The method may include actuating the slider (e.g., to the distal position) to deploy the substantially planar electrode from its sheathed position. The method further includes positioning the substantially planar electrode into electrical communication with a tissue interface separating the target cell from the substantially planar electrode. The method also includes transmitting one or more pulses of electrical energy (e.g., with a voltage source) through the substantially planar electrode at conditions suitable for electrotransfer of the therapeutic agent into the target cell.

In some embodiments, the substantially planar electrode is positioned within about 10 mm (e.g., within 9 mm, 8 mm, 7 mm, 6 mm, 5 mm, 4.5 mm, 4 mm, 3.5 mm, 3 mm, 2.5 mm, 2 mm, 1.5 mm, 1 mm, or 0.5 mm, 0.45 mm, 0.40 mm, 0.35 mm, 0.30 mm, 0.25 mm, 0.20 mm, 0.15 mm, or 0.10 mm) of the tissue interface. The substantially planar electrode may be from 0.1 to about 0.5 mm (e.g., about 0.15 mm, 0.2 mm, 0.25 mm, 0.30 mm, 0.35 mm, 0 40 mm, 0.45 mm, or 0.5 mm), or from about 0.5 mm to 5 mm (e.g., about 0.5 mm, 1 mm, 1.5 mm, 2 mm, 2.5 mm, 3 mm, 3.5 mm, 4 mm, 4.5 mm, or 5 mm) from the tissue interface upon transmission of the one or more pulses. In some embodiments, the proximity of the electrode to the target cell results in a voltage (e.g., potential) at the target cell that is within 20% of the amplitude of the waveform of the applied energy (e.g., within 19%, within 18%, within 17%, within 16%, within 15%, within 14%, within 13%, within 12%, within 11%, within 10%, within 9%, within 8%, within 7% within 6%, within 5%, within 4%, within 3%, within 2%, or within 1% of the amplitude of the waveform of the applied energy). In some embodiments, the substantially planar electrode is within about 1 mm from the tissue interface upon transmission of the one or more pulses. In any of the aforementioned embodiments, the proximity of the electrode to the target cell results in a voltage (e.g., potential) at the target cell that is within 20% of the amplitude of the waveform of the applied energy (e.g., within 19%, within 18%, within 17%, within 16%, within 15%, within 14%, within 13%, within 12%, within 11%, within 10%, within 9%, within 8%, within 7% within 6%, within 5%, within 4%, within 3%, within 2%, or within 1% of the amplitude of the waveform of the applied energy).

The target cell may be within about 5 mm (e.g., 4.5 mm, 4 mm, 3.5 mm, 3 mm, 2.5 mm, 2 mm, 1.5 mm, 1 mm, or 0.5 mm) from the tissue interface. For example, the target cell may be from about 0.01 mm to about 1 mm (e.g., from about 0.01 mm to about 0.1 mm, e.g., about 0.02 mm, 0.03 mm, 0.04 mm, 0.05 mm, 0.06 mm, 0.07 mm, 0.08 mm, 0.09 mm, or 0.1 mm, e.g., from about 0.1 mm to about 1 mm, e.g., about 0.2 mm, 0.3 mm, 0.4 mm, 0.5 mm, 0.6 mm, 0.7 mm, 0.8 mm, 0.9 mm, or 1 mm) from the tissue interface.

It will be appreciated that a variety of suitable electrical parameters and algorithms thereof may be used. The voltage source may be configured to generate an electric field strength, e.g., at a target cell, from about 10 V/cm to about 1,500 V/cm (e.g., from about 10 V/cm to about 100 V/cm, e.g., about 10 V/cm, 20 V/cm, 30 V/cm, 40 V/cm, 50 V/cm, 60 V/cm, 70 V/cm, 80 V/cm, 90 V/cm, or 100 V/cm, e.g., from about 100 V/cm to about 1,000 V/cm, e.g., about 200 V/cm, 300 V/cm, 400 V/cm, 500 V/cm, 600 V/cm, 700 V/cm, 800 V/cm, 900 V/cm, or 1,000 V/cm, e.g., from about 1,000 V/cm to about 1,500 V/cm, e.g., about 1,110 V/cm, 1,200 V/cm, 1,300 V/cm, 1,400 V/cm, or 1,500 V/cm). In some embodiments, the voltage source is be configured to generate an electric field strength, e.g., at a target cell, from about 10 V/cm to about 1,000 V/cm (e.g., from about 10 V/cm to 500 V/cm or from about 500 V/cm to about 1,000 V/cm). In some embodiments, the field strength is from 50 V/cm to 300 V/cm. In some embodiments, the field strength is about 100 V/cm at the target cell.

In some embodiments, the voltage (e.g., potential) at the target cell is from 5 V to 100 V (e.g., from 10 V to 80V, from 15 V to 70 V, from 20 V to 60 V, or from 30 V to 50 V; e.g., about 10 V, about 15 V, about 20 V, about 25 V, about 30 V, about 35 V, about 40 V, about 45 V, about 50 V, about 55 V, about 60 V, about 65 V, or about 70 V). In some embodiments, the voltage (e.g., potential) at the target cell is from 20 V to 60 V. In some embodiments, the voltage (e.g., potential) at the target cell is from 30 V to 50 V, e.g., about 35 V to 45 V. In any of the aforementioned embodiments, close proximity of the electrode to the target cell results in a voltage (e.g., potential) at the target cell that is within 20% of the amplitude of the waveform of the applied energy (e.g., within 19%, within 18%, within 17%, within 16%, within 15%, within 14%, within 13%, within 12%, within 11%, within 10%, within 9%, within 8%, within 7% within 6%, within 5%, within 4%, within 3%, within 2%, or within 1% of the amplitude of the waveform of the applied energy). For instance, a 40 V amplitude pulse from a monopolar intravitreal electrode positioned near the retina may result in a voltage (e.g., potential) of 35 V at a target retinal cell. It will be understood that waveform amplitudes required to achieve a given voltage at a target cell will depend on the electrode configuration (e.g., monopolar vs bipolar), electrode shape, distance between electrode and the target cell, and material properties (e.g., conductivity) of the tissue (e.g., vitreous and retina).

In some embodiments, the total number of pulses of electrical energy are delivered within 1-60 seconds (e.g., within 1-5 seconds, 5-10 seconds, 10-15 seconds, 15-20 seconds, 20-30 seconds, 30-40 seconds, 40-50 seconds, or 50-60 seconds). In some embodiments, the total number of pulses of electrical energy are delivered within 1-20 seconds. For example, the total number of pulses of electrical energy may be delivered within 1-5 seconds, 5-10 seconds, 10-15 seconds, or 15-20 seconds, e.g., 1, 2, 3, 4, 5, 6, 7, 8, 9, 10, 11, 12, 13, 14, 15, 16, 17, 18, 19, or 20 seconds. The pulses of electrical energy may be, e.g., square waveforms. The pulses of electrical energy may have an amplitude from 5 V to 500 V. For example, the pulses of electrical energy may have an amplitude of about 5 V, 10 V, 15 V, 20 V, 25 V, 30 V, 35 V, 40 V, 45 V, 50 V, 60 V, 70 V, 80 V, 90 V, 100 V, 125 V, 150 V, 175 V, 200 V, 225V, 250 V, 275 V, 300 V, 325 V, 350 V, 375 V, 400 V, 425 V, 450 V, 475 V, or 500 V. In some embodiments, the pulses of electrical energy have an amplitude of about 5-250 V (e.g., about 20 V).

In some embodiments, about 1-12 pulses (e.g., 1, 2, 3, 4, 5, 6, 7, 8, 9, 10, 11, or 12 pulses) of electrical energy are transmitted during use. In some embodiments, about 4-12 pulses of electrical energy are transmitted during use.

In some embodiments, each of the pulses of electrical energy is from about 10 ms to about 200 ms. For example, each of the pulses of electrical energy may be about 10 ms, 20 ms, 30 ms, 40 ms, 50 ms, 60 ms, 70 ms, 80 ms, 90 ms, 100 ms, 110 ms, 120 ms, 130 ms, 140 ms, 150 ms, 160 ms, 170 ms, 180 ms, 190 ms, or 200 ms. In some embodiments, each of the pulses of electrical energy is from about 50 ms. In some embodiments, each of the pulses of electrical energy is less than 10 ms. For example, each of the pulses of electrical energy may be from about 10 μs to about 10 ms, e.g., from about 10 μs to about 100 μs, e.g., about 20 μs, 30 μs, 40 μs, 50 μs, 60 μs, 70 μs, 80 μs, 90 μs, or 100 μs, e.g., from about 100 μs to about 1 ms, e.g., about 200 μs, 300 μs, 400 μs, 500 μs, 600 μs, 700 μs, 800 μs, 900 μs, or 1 ms, e.g., from about 1 ms to about 10 ms, e.g., about 2 ms, 3 ms, 4 ms, 5 ms, 6 ms, 7 ms, 8 ms, 9 ms, or 10 ms.

The device may be used in combination with delivery of a therapeutic agent. For example, in some embodiments, the therapeutic agent has been previously administered to the tissue. In other embodiments, the method further includes administering the therapeutic agent concurrently with delivery of a pulse of electrical energy. For example, in some embodiments, the therapeutic agent is administered at the same time as a pulse of electrical energy. In some embodiments, the therapeutic agent is administered concurrently with a pulse of electrical energy. In some embodiments, the therapeutic agent is administered before a pulse of electrical energy. In any of the above embodiments, the device may be configured to deliver the therapeutic agent (e.g., via a channel on or within the sheath. e.g., via a channel between the sheath and the insulator).

The therapeutic agent may be a nucleic acid (e.g., a non-viral nucleic acid (e.g., a naked nucleic acid vector), e.g., a non-viral DNA vector (e.g., a naked DNA vector)). The nucleic acid may be DNA or RNA (e.g., circular DNA (e.g., a naked circular DNA) or circular RNA (e.g., a naked circular RNA)). The nucleic acid may be a vector, e.g., a vector that includes a transgene. The vector may be, e.g., a non-viral vector (e.g., a naked non-viral vector, e.g., a naked non-viral DNA vector).

In some embodiments, the target cell is a cell in the eye, e.g., a retinal cell. The retinal cell may be, e.g., a retinal pigment epithelial (RPE) cell, a photoreceptor cell, or a ganglion cell. The therapeutic agent can be administered, for example, intravitreally, subretinally, suprachoroidally or topically on the eye. The compositions utilized in the methods described herein can be administered locally (e.g., on or in the eye) or systemically (e.g., intravenously). The method of administration can vary depending on various factors (e.g., the compound or composition being administered and the severity of the condition, disease, or disorder being treated). In certain embodiments, the therapeutic agent is delivered via an intravitreal route. In certain embodiments, the therapeutic agent is delivered via a suprachoroidal route. In some embodiments, the device targets the intravitreal space of the eye.

In some embodiments, the device may be used with any method as described herein.

VI. Kits and Articles of Manufacture In another aspect of the invention, an article of manufacture or a kit containing materials useful for the treatments described above is provided. The article of manufacture includes a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a therapeutic agent of the invention (e.g., nucleic acid vector (e.g., a non-viral DNA vector, e.g., a circular DNA vector that lacks a bacterial original of replication, a drug resistance gene, and/or a recombination site)) or a pharmaceutical composition comprising the therapeutic agent of the invention. The label or package insert indicates that the composition is used for treating the disease or disorder of choice. The article of manufacture may further comprise a package insert indicating that the compositions can be used to treat a particular condition (e.g., Usher syndrome type 1B, autosomal recessive bestrophinopathy, autosomal dominant Best vitelliform macular dystrophy, or macular degeneration (e.g., age related macular degeneration (AMD)). Alternatively, or additionally, the article of manufacture may further comprise a second container comprising a pharmaceutically acceptable carrier, such as bacteriostatic water for injection (BWFI), phosphate-buffered saline, Ringer's solution, dextrose solution, or any of the pharmaceutically acceptable carriers disclosed above. It may further include other materials desirable from a commercial and user standpoint, including other buffers, diluents, filters, needles, and syringes.

In particular instances of the invention, provided is a kit that includes (i) any one or more of the materials described above (e.g., any of the aforementioned therapeutic agents of the invention and/or one or more pharmaceutically acceptable carriers) and (ii) one or more elements of an energy delivery device (e.g., a device including an electrode for transmitting an electric field to a tissue (e.g., retina), such as any suitable devices or systems described above). In some embodiments, provided herein is a kit that includes a therapeutic agent of the invention (e.g., a nucleic acid vector (e.g., a non-viral DNA vector, e.g., a circular DNA vector that lacks a bacterial original of replication, a drug resistance gene, and/or a recombination site)) and an electrode. In some embodiments, provided herein is a kit that includes a pharmaceutical composition comprising a therapeutic agent of the invention (e.g., a nucleic acid vector (e.g., a non-viral DNA vector, e.g., a circular DNA vector that lacks a bacterial original of replication, a drug resistance gene, and/or a recombination site)) and an electrode.

### EXAMPLES

The following are non-limiting examples of methods and compositions described above. The following examples also provide non-limiting methods for modeling and using the devices described above. A skilled artisan will recognize that variations of the examples below are also encompassed by the description herein.

### Example 1. Computational Modeling of a Substantially Planar Electrode

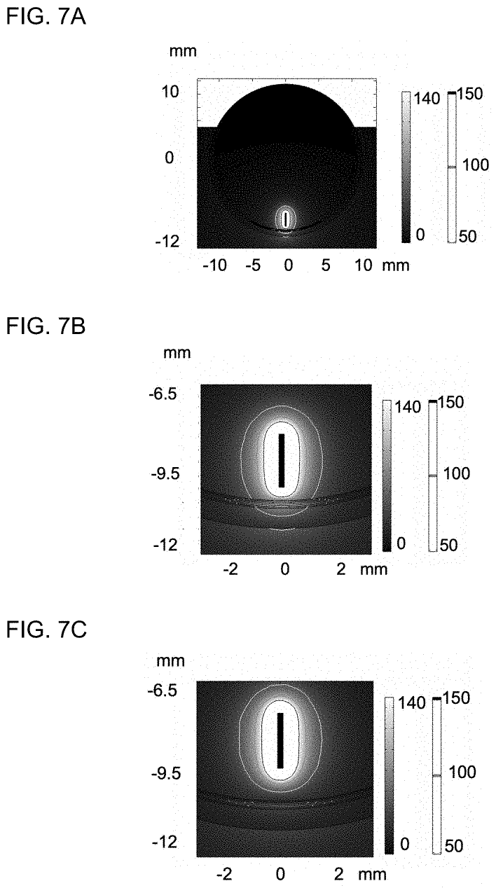

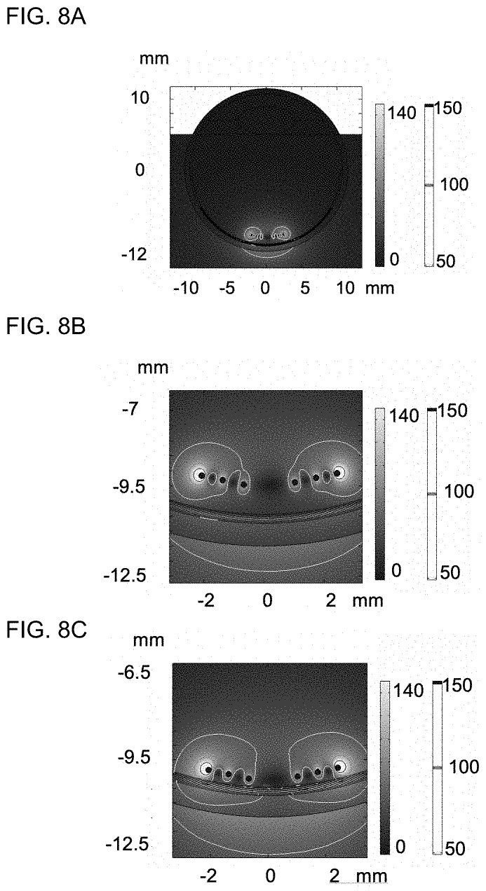

An electric field distribution simulation was conducted to compare the effects of an electric field transmitted by a substantially planar electrode on retinal tissue relative to the effects of an electric field transmitted by a needle electrode on retinal tissue while each electrode design is positioned for electrotransfer of a therapeutic agent to the retina (i.e., contacting the vitreous humor anterior to the retina). FIGS. 7A-7C show the needle electrode, whereas FIGS. 8A-8C show the substantially planar electrode. Each electrode is monopolar.

FIGS. 7A-7C show a transverse cross-section of an eye containing the needle electrode at the posterior portion of the vitreous humor (shown on the graph as the lower segment of the circle in FIG. 7A). Upon application of a voltage, the needle electrode produces an elliptical electric field along the axis of its sheath. When the distal end of the needle electrode was positioned 0.25 mm from the vitreous humor-retina interface (FIGS. 7A and 7B), the volume of retinal tissue experiencing an electric field strength of >50 V/cm was 0.5 mm³; the volume of retinal tissue experiencing an electric field strength of >100 V/cm was 0.15 mm³; and the volume of retinal tissue experiencing an electric field strength of >150 V/cm was 0.075 mm³. As shown in FIG. 7C, when the distal end of the needle electrode was positioned further from the vitreous humor-retina interface (0.95 mm from the vitreous humor-retina interface), the volume of retinal tissue experiencing an electric field strength of >50 V/cm decreased to 0.4 mm³ and none of the retinal tissue experienced an electric field strength of >100 V/cm. Thus, anterior displacement of the needle electrode by 0.7 mm resulted in 100% decrease in retinal volume experiencing an electric field strength of at least 100 V/cm.

FIGS. 8A-8C show that the electric field strength experienced by the retina upon transmission by a substantially planar electrode is substantially less sensitive to electrode position. In this simulation, anterior displacement of the substantially planar electrode by 0.7 mm resulted in just 8% decrease in retinal volume experiencing an electric field strength of at least 100 V/cm. When the distal end of the substantially planar electrode was positioned 0.25 mm from the vitreous humor-retina interface (FIG. 8C), the volume of retinal tissue experiencing an electric field strength of >50 V/cm was 1.87 mm³; the volume of retinal tissue experiencing an electric field strength of >100 V/cm was 1.11 mm³; and the volume of retinal tissue experiencing an electric field strength of >150 V/cm was 0.77 mm³. As shown in FIG. 8B, when the distal end of the substantially planar electrode was positioned further from the vitreous humor-retina interface (0.95 mm from the vitreous humor-retina interface), the volume of retinal tissue experiencing an electric field strength of >50 V/cm was 2.27 mm³; the volume of retinal tissue experiencing an electric field strength of >100 V/cm was 1.02 mm³; and none of the retinal tissue experienced an electric field strength of >150 V/cm.

Thus, in addition to the improvement in tolerance to changing electrode position relative to the retina, the substantially planar electrode design confers access to a larger volume of retina by the transmitted electric field, relative to the needle electrode design.

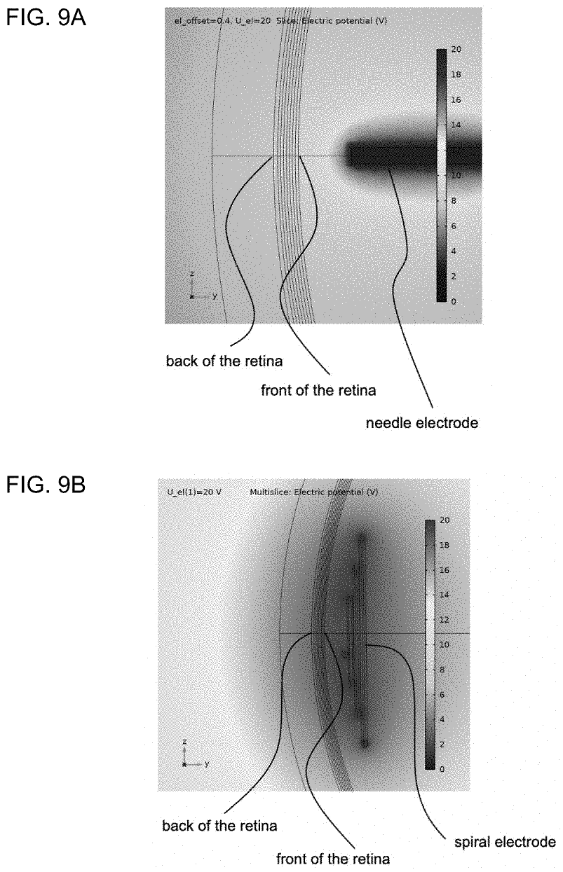

FIGS. 9A and 9B show that the potential at the retina more closely matches the voltage at the electrode when the voltage is applied using a spiral electrode (FIG. 9B) relative to a needle electrode (FIG. 9A). When the distal end of a needle electrode having a potential of 20 V was positioned 0.4 mm from the vitreous humor-retina interface (FIG. 9A), the potential at the front of the retina was 10.8 V, and the potential at the back of the retina (choroid-retina interface) was 9.24. In comparison, when a spiral electrode having a potential of 20 V was positioned 0.4 mm from the vitreous humor-retina interface (FIG. 9B), the potential at the front of the retina was 19.0 V, and the potential at the back of the retina (choroid-retina interface) was 17.9 V. This model demonstrates how a planar electrode design can improve consistency of energy delivery and electrotransfer across a volume of target tissue.

### Example 2. Delivery of a Nucleic Acid Vector to the Retina Using a Device Having a Substantially Planar Electrode

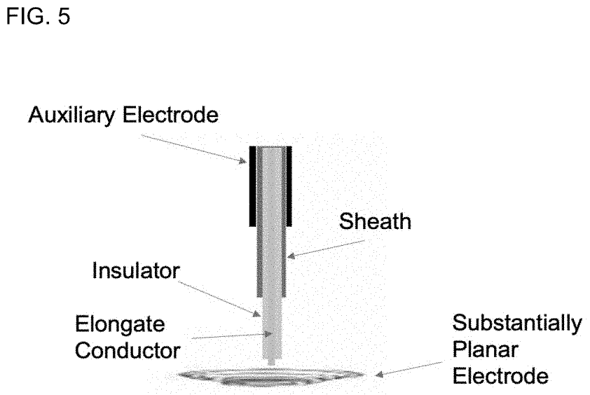

A bipolar electrode device as shown in FIG. 5 is used to deliver a nucleic acid vector to a population of retinal pigment epithelial cells in an individual following diagnosis of the patient with an inherited retinal disorder characterized by a mutation in a gene encoding a retinal protein. The patient had been prescribed a pharmaceutical composition containing a non-viral DNA vector encoding the retinal protein, and the pharmaceutical composition containing, for example, 20 to 150 (e.g., 50 to 150) microliters is administered to the patient's eye via subretinal or intravitreal injection. As part of the same procedure, a device having an elongate conductor retracted within a sheath is inserted into the vitreous humor of the eye containing the non-viral DNA vector. Using an actuator, an operator slides the elongate conductor distally, relative to the sheath, until the sheath is in its distal position, thereby forming a substantially planar electrode within the vitreous humor. Using a surgical microscope as a visual guide, the operator positions the substantially planar electrode in a substantially co-planar orientation over the target area of the retina, offset from the vitreous humor-retina interface by about 0.5 mm. The operator transmits eight 50 V, 20 ms pulses through the electrode over the course of eight seconds at one pulse per second. Alternatively, an operator may choose to transmit eight 20 V, 20 ms pulses. The operator retracts the substantially planar electrode proximally into the sheath and removes the device from the patient's eye. The procedure is concluded, and the patient is monitored for improved expression of the gene delivered by the procedure over the subsequent weeks and months.

### Example 3: Electrotransfer of a Synthetic Circular DNA Vector Encoding GFP in Pig Retina

A supercoiled, synthetic covalently closed circular (C³) DNA vector encoding GFP and lacking a bacterial origin of replication, drug resistance gene, and recombination site (C³-GFP), was produced using Phi29 polymerase-mediated rolling circle amplification in a cell-free process following methods taught in International Patent Publication WO 2019/178500.

225 ug of vector was administered by single bilateral subretinal injection in two subretinal blebs (75 uL each) in each eye of Gottingen minipigs on Day 1 of the study. Briefly, animals were anesthetized and placed in lateral recumbency. Topical Proparacaine was applied to the eye. The conjunctival fornices were flushed with a 1:50 dilution of betadine solution/saline and the eyelid margins swabbed with undiluted 5% betadine solution. The eye was draped, and a wire eyelid speculum placed. A caliper was used to mark spots 3.0 mm posterior to the limbus on the superotemporal and superonasal sclera. Bipolar cautery was used to cauterize the sclera under the marked spots, followed by topical application of undiluted 5% betadine solution. Scleral fixation forceps was used to fix the globe position while a microvitreoretinal blade with a 25 g valved cannula was inserted at each marked spot, through the conjunctiva and sclera, and advanced into the vitreous humor. A trocar was positioned to face the posterior axis of the globe, and then retracted to leave the scleral port in place. A 31 g needle was then inserted tangentially through the limbus and into the anterior chamber to remove 75 μL aqueous humor. A direct contact surgical lens was placed on the cornea with sterile coupling gel. An endoilluminator probe was inserted through one of the scleral ports to facilitate direct visualization of the posterior segment through the microscope. A subretinal injection cannula was inserted through the second port and advanced into the mid-vitreous. The small diameter injection cannula was then advanced until it contacts the retinal surface. The dosing solution was then slowly delivered to induce and fill a subretinal bleb. Upon visualization of appropriate bleb formation, the injection was continued to deliver the entire dose volume (75 μL per bleb) into the subretinal space. Two injection blebs were administered within the nasal and temporal regions. Once the injection doses were delivered, the injection cannula and endoilluminator probe were removed from the scleral ports, and the contact lens removed from the cornea. Once the PEF was delivered, the scleral ports were removed.

Group-specific methods for pulsed electric field (PEF) conditions and results are described below.

### Subretinal PEF by Monopolar Needle Electrode

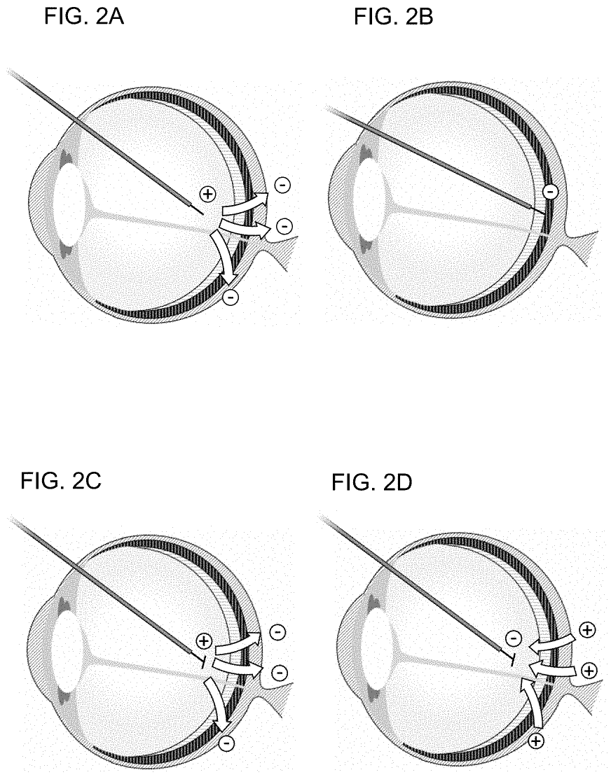

Within 5 minutes of the injection, a monopolar needle electrode (negative electrode, length from 0.2 to 2 mm, diameter sized to fit through a 25-gauge trocar) was placed within the subretinal bleb (as represented by FIG. 2B), and eight 20-ms electrical pulses were transmitted at 20 V over eight seconds. Average current measured at these conditions was 13.7 mA. Optical coherence tomography (OCT) and confocal scanning laser tomography (cSLO) were conducted at pretreatment and at Day 7. At Day 7, animals were euthanized, and eyes were dissected to collect the retina and retinal pigment epithelium (RPE) and choroid for staining.

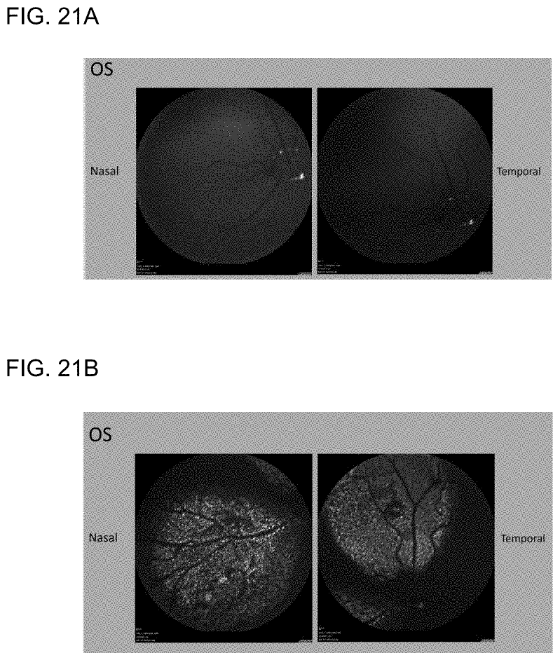

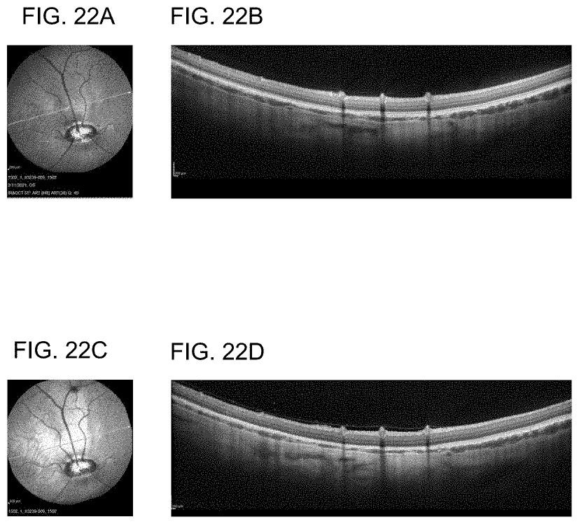

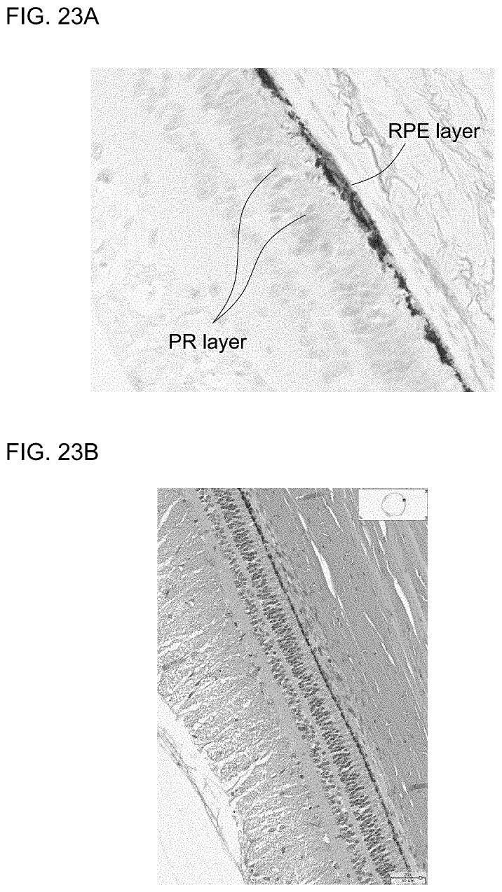

Confocal scanning laser ophthalmoscopy (cSLO) images from Day 7 indicated widespread and homogeneous GFP fluorescence within the sub-retinal blebs (FIG. 21B) compared to baseline (FIG. 21A). Optical coherence tomography (OCT) images suggested that the transfection was safe; no structural changes or inflammation were detected (FIGS. 22A-22D). H&E staining was consistent in showing no structural changes (FIG. 23B). Histological images (IHC) showed GFP expression in both photoreceptors (PR) and retinal pigment epithelial (RPE) cells (FIG. 23A).

### Intravitreal PEF by Monopolar Needle Electrodes

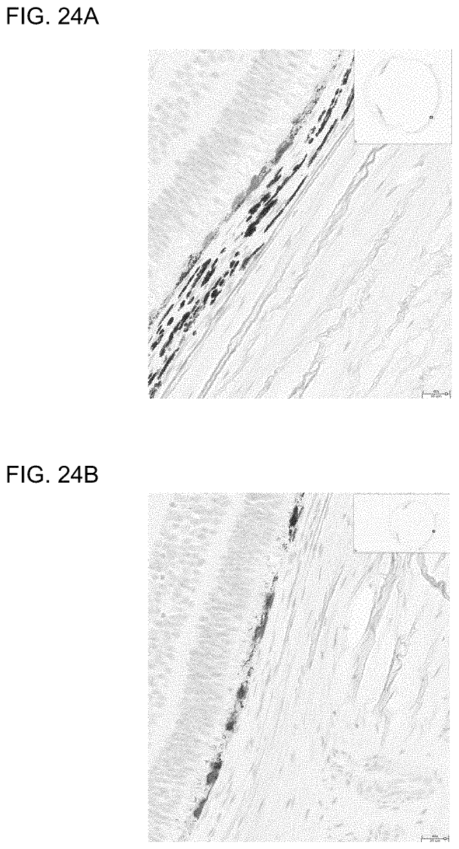

A monopolar needle electrode (positive electrode; length from 0.2 to 2 mm, diameter sized to fit through a 25-gauge trocar) was positioned in the vitreous such that the distal end of the electrode was within 1 mm from the retina (as represented by FIG. 2A). Eight 20-ms electrical pulses were transmitted at 40 V over eight seconds. Average current measured at these conditions was 26.7 mA. At Day 6, animals were euthanized, and eyes were dissected to collect the retina and retinal pigment epithelium (RPE) and choroid for staining. GFP expression was observed in the RPE layer (FIGS. 24A and 24B). Analogous PEF methods with a negative electrode placed in the vitreous resulted in negative GFP staining in the RPE layer (data not shown).

### Subretinal PEF by Bipolar Needle Electrode

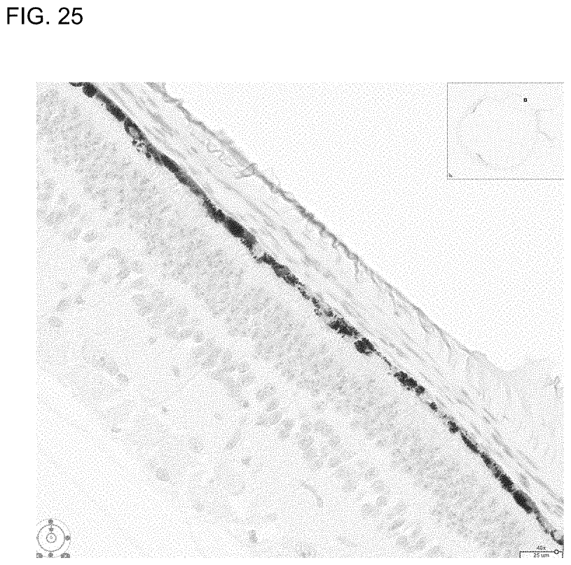

A bipolar needle electrode having a negative electrode at its distal end and a positive electrode on the needle proximal to the distal end was positioned such that the negative electrode was in the subretinal bleb and the positive electrode was in the vitreous. Eight 20-ms electrical pulses were transmitted at 40 V over eight seconds. At Day 6, animals were euthanized, and eyes were dissected to collect the retina and retinal pigment epithelium (RPE) and choroid for staining. GFP expression was observed in the RPE layer (FIG. 25).

### Intravitreal PEF by Monopolar Planar Electrode

A monopolar spiral electrode as shown in FIG. 6 was positioned in the vitreous humor within 1 mm from the target retinal tissue and a dispersive patch was placed on the abdomen of the animal. +40V (as represented by FIG. 2C) or −40V (as represented by FIG. 2D) electrical energy was transmitted from the monopolar electrode in 8 pulses, each pulse having a duration of 20 ms. Average current measured at these conditions was 32.1 mA. At Day 7, animals were euthanized, and eyes were dissected to collect the retina and retinal pigment epithelium (RPE) and choroid for staining.

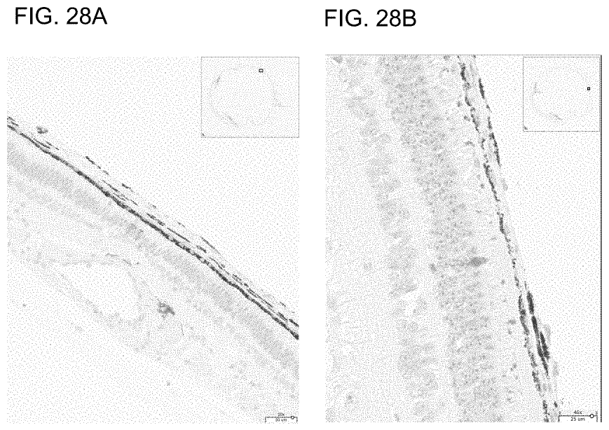

In both conditions, minimal retinal degeneration was observed. In eyes receiving+40V pulses (intravitreal positive electrode), GFP staining was observed in photoreceptor cells in the retina (FIGS. 28A and 28B). In eyes receiving −40V pulses (intravitreal negative electrode), GFP staining was negative (data not shown).

### Controls

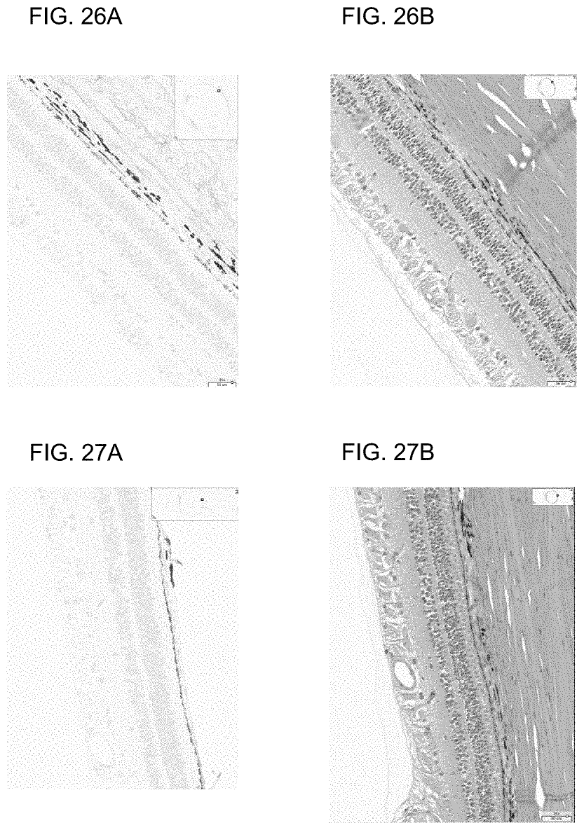

As controls, eyes were injected subretinally with C³-GFP without electrotransfer by pulsed electric field (FIGS. 26A and 26B) and injected subretinally with PBS with electrotransfer by pulsed electric field (FIGS. 27A and 27B). In eyes injected with C³-GFP without electrotransfer, no significant GFP labeling in the RPE was observed (FIG. 26A). No non-specific labeling was observed in eyes treated with PBS (FIG. 27A).

### Example 4: Persistent Expression of Synthetic Circular DNA Vectors in iRPE Cells

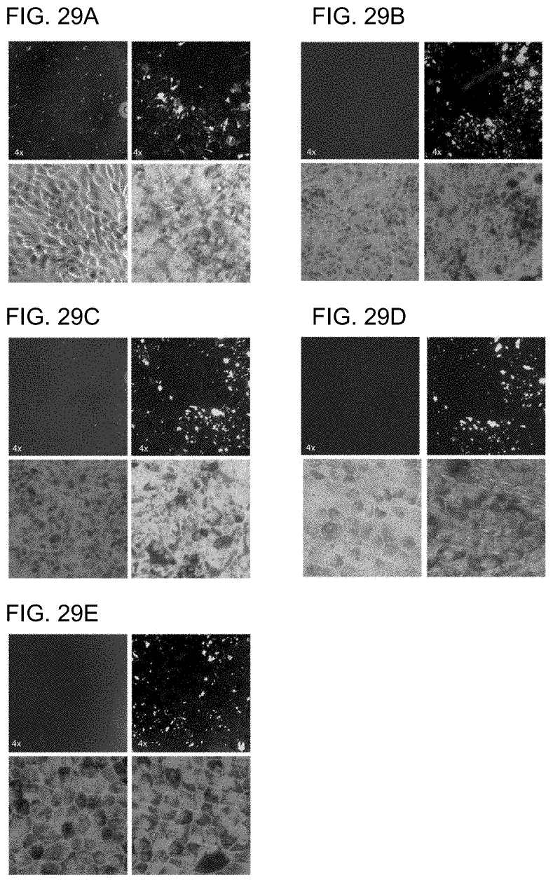

To assess persistence of expression of synthetic covalently closed circular (C³) DNA vectors in retinal cells, induced retinal pigment epithelial (iRPE) cells were generated according to known methods, transfected with and without pulsed electric field at Day 1, and monitored for GFP expression over time. Synthetic C³ DNA vectors encoding GFP were those described in Example 1. iRPE cells were seeded on 6.5 mm trans-well plates, and 20 ug synthetic C³ DNA vector was added in 120 uL total volume per trans-well (upper chamber). A bipolar plate electrode assembly was positioned above and below the cell membrane in each well at a 4 mm distance between electrode poles, and two pulses of 300-450 V were applied, each having a pulse duration of 5 or 20 seconds. Images were taken at Day 4 (FIG. 29A), Day 21 (FIG. 29B), Day 32 (FIG. 29C), Day 40 (FIG. 29D), and Day 49 (FIG. 29E). GFP expression was observed in cells transfected by electrotransfer at all timepoints, with no indication of decline.

### Example 5: Expression of Human ABCA4 mRNA in Pig Retina by In Vivo Electrotransfer

A synthetic covalently closed circular (C³) DNA vector encoding full-length, human ABCA4 driven by a CAG promoter and lacking a bacterial origin of replication, drug resistance gene, and recombination site (C³-ABCA4; 8656 bp; SEQ ID NO: 19) was produced using Phi29 polymerase-mediated rolling circle amplification in a cell-free process following methods generally taught in International Patent Publication WO 2019/178500. Naked C³-ABCA4 was administered to pig by subretinal injection (225 ug/eye), and subretinal PEF was administered using a monopolar needle electrode (as represented by FIG. 2B). 20V electrical energy was transmitted from the monopolar electrode (negative electrode) in 8 pulses, each pulse having a duration of 20 ms. Eyes were harvested and neuroretina (NR) and RPE/choroid layers were isolated. RNA was isolated from tissues and mRNA levels for ABCA4 transgene and endogenous pig ABCA4 were quantified by qPCR using standard methods.

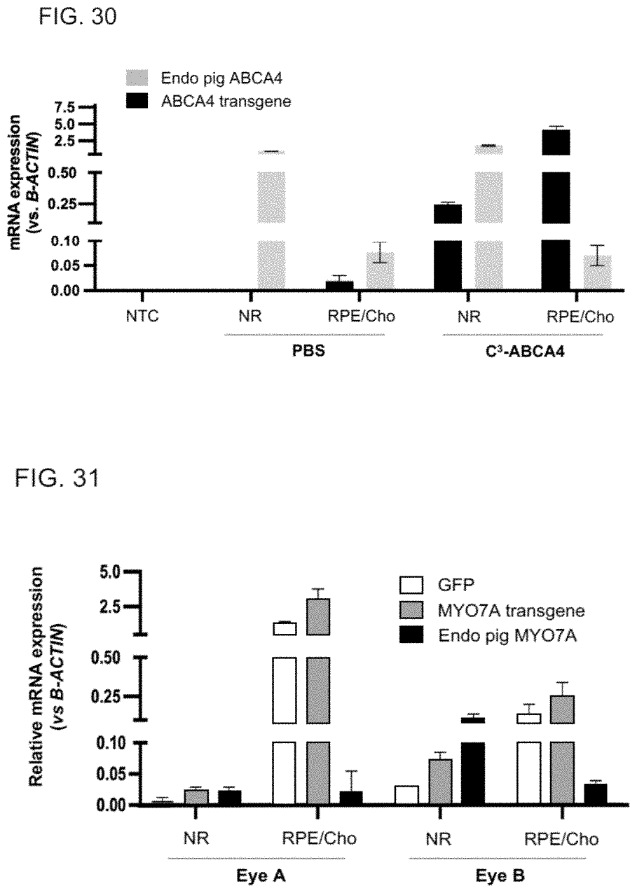

As shown in FIG. 30, ABCA4 transgene mRNA expression was detected in both the NR layer, which contains photoreceptors, and the RPE/choroid layer. In general, higher ABCA4 transgene mRNA expression was detected in the RPE/choroid layer relative to the neuroretina layer. This study shows that administration of C³-ABCA4 by PEF-mediated electrotransfer resulted in ABCA4 transgene expression in vivo.

### Example 6: Expression of Human MYO7A mRNA in Pig Retina by In Vivo Electrotransfer

A synthetic C³ DNA vector encoding full-length, human MYO7A lacking a bacterial origin of replication, drug resistance gene, and recombination site (C³-MYO7A) was produced using Phi29 polymerase-mediated rolling circle amplification in a cell-free process following methods generally taught in International Patent Publication WO 2019/178500. Naked C³-MYO7A was administered by subretinal injection (225 ug DNA per eye; 2.53×10¹³ vector copies per eye), and subretinal PEF was administered using a monopolar needle electrode (as represented by FIG. 2B). 20V electrical energy was transmitted from the monopolar electrode (negative electrode) in 8 pulses, each pulse having a duration of 20 ms. Eyes were harvested and neuroretina (NR) and RPE/choroid layers were isolated. RNA was isolated from tissues and mRNA levels for ABCA4 transgene and endogenous pig ABCA4 were quantified by qPCR using standard methods.

As shown in FIG. 31, MYO7A transgene mRNA expression was detected in both the NR layer, which contains photoreceptors, and the RPE/choroid layer. Broadly, higher MYO7A transgene mRNA expression was detected in the RPE/choroid layer relative to the neuroretina layer. This study shows that administration of C³-MYO7A by PEF-mediated electrotransfer resulted in ABCA4 transgene expression in vivo.

### Example 7: Expression of Human ABCA4 Protein in Pig Retina by In Vivo Electrotransfer

A synthetic covalently closed circular (C³) DNA vector encoding human ABCA4 driven by a CAG promoter and lacking a bacterial origin of replication, drug resistance gene, and recombination site (C³-ABCA4; 8656 bp; SEQ ID NO: 19), was produced using Phi29 polymerase-mediated rolling circle amplification in a cell-free process following methods generally taught in International Patent Publication WO 2019/178500. C³-ABCA4 was formulated in solution at a concentration of 1.5 mg/mL. Naked C³-ABCA4 was administered to by injecting two blebs of 75 uL each into the subretinal space of Gottingen Minipigs (225 ug DNA per eye; 2.53×10¹³ vector copies per eye). After injection, a monopolar needle electrode was place within each subretinal bleb, and eight 20-ms electrical pulses were transmitted at 20V. At Day 6, animals were euthanized, and eyes were dissected to collect the retina and RPE and choroid for staining.

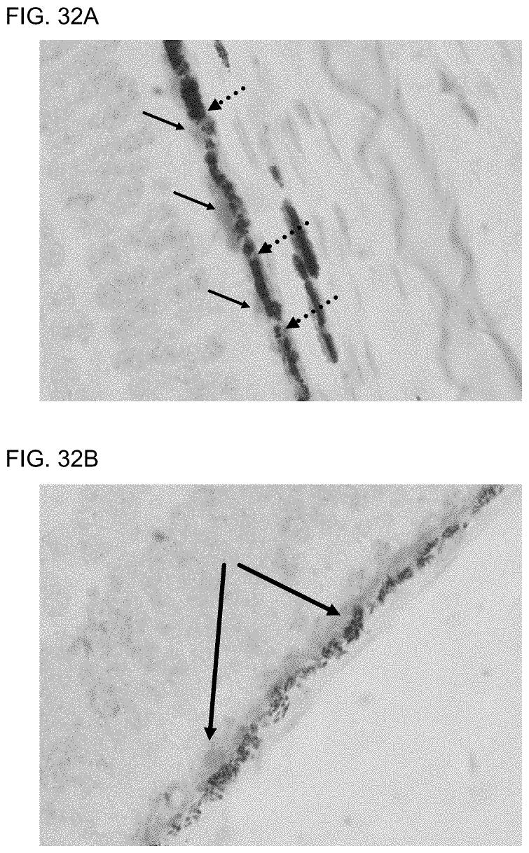

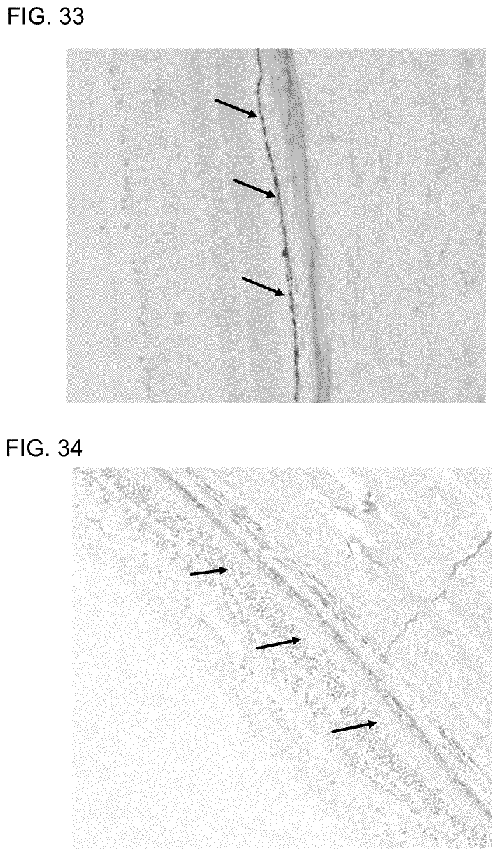

Widespread human ABCA4 protein expression was observed in the photoreceptor layer, adjacent to the RPE (FIG. 32A). Moreover, human ABCA4 protein was expressed in the photoreceptor outer segments (FIG. 32B, showing co-localization with rhodopsin). These data indicate that in vivo electrotransfer of C³-ABCA4 in pigs led to widespread expression of human ABCA4 protein in the desired cell type (PR) and at the desired subcellular location within those cells (outer segments). To confirm clinical feasibility, FIGS. 32 and 33 show identical localization of human ABCA4 transgene in pigs (FIG. 33) as human endogenous ABCA4 in the human eye (FIG. 34).

### Example 8: Expression Comparison of C³-ABCA4 with Plasmid-ABCA4 in iRPE Cells

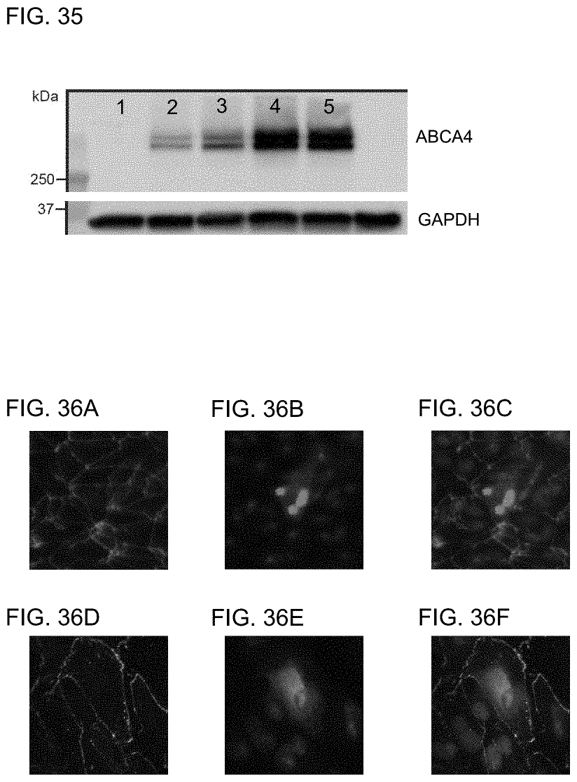

Induced retinal pigment epithelial (iRPE cells) were generated according to known methods and transfected in vitro with ABCA4 encoded by plasmid or synthetic circular DNA vector produced by Phi29 polymerase-mediated rolling circle amplification in a cell-free process. Briefly, iRPE cells were seeded in laminin-coated 6-well plates and cultured for 48 hours to 100% confluence. Cells were lifted with TrypLE, counted, and replated at >2.5×10⁵ cells per 24-well. DNA vector was added at 1 ug/well, and cells were electroporated using a Neon transfection system at 1100 V; 20 ms. Cells were incubated for 48 hours before antibody staining. Protein expression analysis revealed that synthetic circular DNA vector expressed higher amounts of ABCA4 protein compared to plasmid (FIG. 35). Representative fluorescence images showing ABCA4 expression by synthetic circular DNA vector are shown in FIGS. 36A-36C, compared to expression by plasmid vector, shown in FIGS. 36D-36F.

### Example 9: Expression Comparison of C³-MYO7A with Plasmid-MYO7A in iRPE Cells

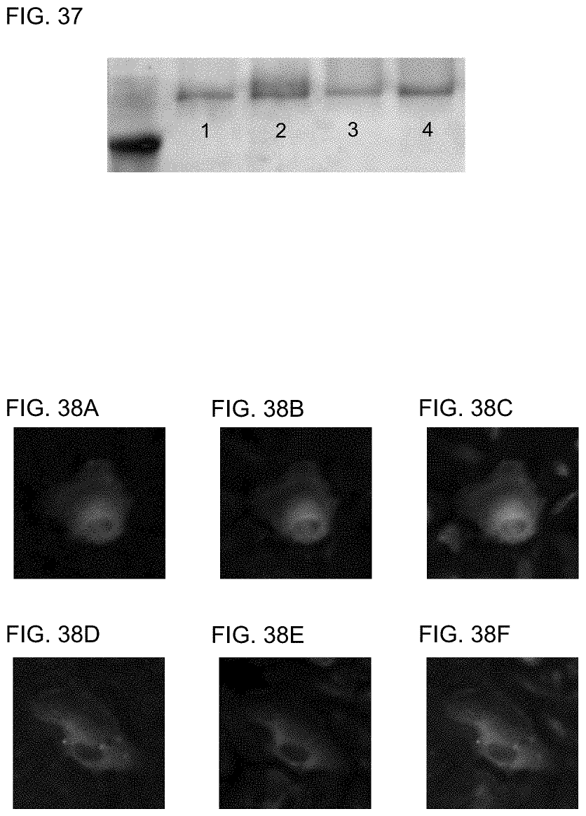

iRPE cells were generated according to known methods and transfected in vitro with MYO7A encoded by plasmid or synthetic circular DNA vector produced by Phi29 polymerase-mediated rolling circle amplification in a cell-free process. Briefly, iRPE cells were seeded in laminin-coated 6-well plates and cultured for 48 hours to 100% confluence. Cells were lifted with TrypLE, counted, and replated at >2.5×10⁵ cells per 24-well. DNA vector was added at 1 ug/well, and cells were electroporated using a Neon transfection system at 1100 V; 20 ms. Cells were incubated for 48 hours before antibody staining. Protein expression analysis (FIG. 37) revealed that synthetic circular DNA vector (lane 4) expressed higher amounts of MYO7A protein compared to a plasmid encoding the same MYO7A transgene (lanes 3). Representative fluorescence images showing MYO7A expression by synthetic circular DNA vector are shown in FIGS. 38A-38C, compared to expression by plasmid vector, shown in FIGS. 38D-38F.

### Example 10: Treatment of Stargardt Disease by Subretinal DNA Injection and Subretinal PEF Administration

The patient is an adult human with biallelic ABCA4 mutations causing retinal degeneration due to Stargardt disease.C³-ABCA4 as described in Example 7 is provided in naked form in an aqueous pharmaceutical composition and loaded into a subretinal delivery device. 150 μl of pharmaceutical composition is administered subretinally to each eye of the patient (225 μg DNA per eye; 2.53×10¹³ vector copies per eye).

The patient is prepared for pulsed electric field (PEF) therapy. Within thirty minutes after subretinal injection of the circular DNA vector, an energy delivery device having an elongate wire electrode within a sheath is inserted into the vitreous humor of each eye, as shown in FIG. 2A. Using a surgical microscope as a visual guide, the exposed electrode is positioned wholly within the vitreous humor, about 0.5 mm from the retina, centered at the macula. Eight pulses of 40-Volt waveforms are transmitted by the electrode, each pulse having a duration of 20 milliseconds. The electrode is removed from each eye, and the procedure is complete.

After the procedure, the patient is monitored weekly to assess progression of retinal degeneration. Toward this end, visual acuity and visual field are monitored using standard tests. OCT is conducted to assess changes in retinal structure.

### Example 11: Treatment of Usher Syndrome Type 1B by Subretinal DNA Injection and Intravitreal PEF Administration

The patient is an adult human with allelic MYO7A mutations causing retinal degeneration due to Usher syndrome 1B.

Covalent closed circular DNA vector encoding MYO7A is produced using a cell free method by phi-29-mediated rolling circle amplification adapted from the method described in International Patent Publication No. WO 2021/055760. The circular DNA vector is provided in naked form in an aqueous buffered pharmaceutical composition and loaded into a subretinal delivery device.

100 μl of pharmaceutical composition is administered subretinally to each eye of the patient. The patient is prepared for PEF therapy. Within thirty minutes after subretinal injection of the circular DNA vector, an energy delivery device having an elongate wire electrode within a sheath is inserted into the vitreous humor of each eye, as shown in FIG. 2A. Using a surgical microscope as a visual guide, the exposed electrode is positioned wholly within the vitreous humor, about 1 mm from the retina, centered at the macula. Eight pulses of 40-Volt waveforms are transmitted by the electrode, each pulse having a duration of 20 milliseconds. The electrode is removed from each eye, and the procedure is complete.

After the procedure, the patient is monitored weekly to assess progression of retinal degeneration. Toward this end, visual acuity and visual field are monitored using standard tests. OCT is conducted to assess changes in retinal structure.

### Example 12: Treatment of Usher Syndrome Type 1B by Suprachoroidal DNA and PEF Administration

The patient is an adult human with retinal degeneration due to allelic MYO7A mutations causing retinal degeneration due to Ushers syndrome 1B.

Covalent closed circular DNA vector encoding MYO7A is synthesized using a cell free method by phi-29-mediated rolling circle amplification adapted from the method described in International Patent Publication No. WO 2021/055760. The circular DNA vector is provided in naked form in an aqueous buffered pharmaceutical composition and loaded into a delivery device having a microneedle configured for suprachoroidal administration, such as a device described in International Patent Publication No. WO 2014/074823.

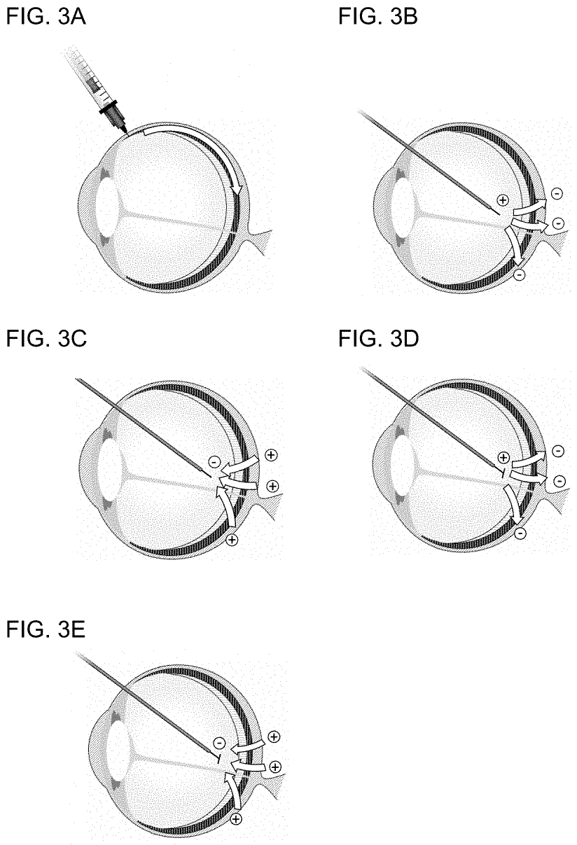

As illustrated in FIG. 3A, 100 μl of pharmaceutical composition is administered suprachoroidally to each eye of the patient. The circular DNA vector migrates through the suprachoroidal space toward the back of the eye, where it occupies the extracellular space surrounding the retina (in the retina and/or in the suprachoroidal space adjacent to the retina).

The patient is prepared for pulsed electric field therapy. Within thirty minutes after suprachoroidal injection of the circular DNA vector, an energy delivery device having an elongate wire electrode within a sheath is inserted into the vitreous humor of each eye, as shown in FIGS. 3B-3E. Using a surgical microscope as a visual guide, the exposed electrode is positioned wholly within the vitreous humor, about 1 mm from the retina, centered at the macula. Eight pulses of 40-Volt waveforms are transmitted by the electrode, each pulse having a duration of 20 milliseconds. The electrode is removed from each eye, and the procedure is complete.

After the procedure, the patient is monitored weekly to assess progression of retinal degeneration. Toward this end, visual acuity and visual field are monitored using standard tests. OCT is conducted to assess changes in retinal structure.

TABLE 1
Sequences

| SEQ | | |
|---|---|---|
| ID NO: | Gene | Sequence |
| 1 | MYO7A | ATGGTGATTCTTCAGCAGGGGGACCATGTGTGGATGGACCTGAGATTGGGGCAGGAGTTC |
| | DNA | GACGTGCCCATCGGGGCGGTGGTGAAGCTCTGCGACTCTGGGCAGGTCCAGGTGGTGGAT |
| | | GATGAAGACAATGAACACTGGATCTCTCCGCAGAACGCAACGCACATCAAGCCTATGCAC |
| | | CCCACGTCGGTCCACGGCGTGGAGGACATGATCCGCCTGGGGGACCTCAACGAGGCGGGC |
| | | ATCTTGCGCAACCTGCTTATCCGCTACCGGGACCACCTCATCTACACGTATACGGGCTCC |
| | | ATCCTGGTGGCTGTGAACCCCTACCAGCTGCTCTCCATCTACTCGCCAGAGCACATCCGC |
| | | CAGTATACCAACAAGAAGATTGGGGAGATGCCCCCCCACATCTTTGCCATTGCTGACAAC |
| | | TGCTACTTCAACATGAAACGCAACAGCCGAGACCAGTGCTGCATCATCAGTGGGGAATCT |
| | | GGGGCCGGGAAGACGGAGAGCACAAAGCTGATCCTGCAGTTCCTGGCAGCCATCAGTGGG |
| | | CAGCACTCGTGGATTGAGCAGCAGGTCTTGGAGGCCACCCCCATTCTGGAAGCATTTGGG |
| | | AATGCCAAGACCATCCGCAATGACAACTCAAGCCGTTTCGGAAAGTACATCGACATCCAC |
| | | TTCAACAAGCGGGGCGCCATCGAGGGCGCGAAGATTGAGCAGTACCTGCTGGAAAAGTCA |
| | | CGTGTCTGTCGCCAGGCCCTGGATGAAAGGAACTACCACGTGTTCTACTGCATGCTGGAG |
| | | GGTATGAGTGAGGATCAGAAGAAGAAGCTGGGCTTGGGCCAGGCCTCTGACTACAACTAC |
| | | TTGGCCATGGGTAACTGCATAACCTGTGAGGGCCGGGTGGACAGCCAGGAGTACGCCAAC |
| | | ATCCGCTCCGCCATGAAGGTGCTCATGTTCACTGACACCGAGAACTGGGAGATCTCGAAG |
| | | CTCCTGGCTGCCATCCTGCACCTGGGCAACCTGCAGTATGAGGCACGCACATTTGAAAAC |
| | | CTGGATGCCTGTGAGGTTCTCTTCTCCCCATCGCTGGCCACAGCTGCATCCCTGCTTGAG |
| | | GTGAACCCCCCAGACCTGATGAGCTGCCTGACTAGCCGCACCCTCATCACCCGCGGGGAG |
| | | ACGGTGTCCACCCCACTGAGCAGGGAACAGGCACTGGACGTGCGCGACGCCTTCGTAAAG |
| | | GGGATCTACGGGCGGCTGTTCGTGTGGATTGTGGACAAGATCAACGCAGCAATTTACAAG |
| | | CCTCCCTCCCAGGATGTGAAGAACTCTCGCAGGTCCATCGGCCTCCTGGACATCTTTGGG |
| | | TTTGAGAACTTTGCTGTGAACAGCTTTGAGCAGCTCTGCATCAACTTCGCCAATGAGCAC |
| | | CTGCAGCAGTTCTTTGTGCGGCACGTGTTCAAGCTGGAGCAGGAGGAATATGACCTGGAG |
| | | AGCATTGACTGGCTGCACATCGAGTTCACTGACAACCAGGATGCCCTGGACATGATTGCC |
| | | AACAAGCCCATGAACATCATCTCCCTCATCGATGAGGAGAGCAAGTTCCCCAAGGGCACA |
| | | GACACCACCATGTTACACAAGCTGAACTCCCAGCACAAGCTCAACGCCAACTACATCCCC |
| | | CCCAAGAACAACCATGAGACCCAGTTTGGCATCAACCATTTTGCAGGCATCGTCTACTAT |
| | | GAGACCCAAGGCTTCCTGGAGAAGAACCGAGACACCCTGCATGGGGACATTATCCAGCTG |
| | | GTCCACTCCTCCAGGAACAAGTTCATCAAGCAGATCTTCCAGGCCGATGTCGCCATGGGC |
| | | GCCGAGACCAGGAAGCGCTCGCCCACACTTAGCAGCCAGTTCAAGCGGTCACTGGAGCTG |
| | | CTGATGCGCACGCTGGGTGCCTGCCAGCCCTTCTTTGTGCGATGCATCAAGCCCAATGAG |
| | | TTCAAGAAGCCCATGCTGTTCGACCGGCACCTGTGCGTGCGCCAGCTGCGGTACTCAGGA |
| | | ATGATGGAGACCATCCGAATCCGCCGAGCTGGCTACCCCATCCGCTACAGCTTCGTAGAG |
| | | TTTGTGGAGCGGTACCGTGTGCTGCTGCCAGGTGTGAAGCCGGCCTACAAGCAGGGCGAC |
| | | CTCCGCGGGACTTGCCAGCGCATGGCTGAGGCTGTGCTGGGCACCCACGATGACTGGCAG |
| | | ATAGGCAAAACCAAGATCTTTCTGAAGGACCACCATGACATGCTGCTGGAAGTGGAGCGG |
| | | GACAAAGCCATCACCGACAGAGTCATCCTCCTTCAGAAAGTCATCCGGGGATTCAAAGAC |
| | | AGGTCTAACTTTCTGAAGCTGAAGAACGCTGCCACACTGATCCAGAGGCACTGGCGGGGT |
| | | CACAACTGTAGGAAGAACTACGGGCTGATGCGTCTGGGCTTCCTGCGGCTGCAGGCCCTG |
| | | CACCGCTCCCGGAAGCTGCACCAGCAGTACCGCCTGGCCCGCCAGCGCATCATCCAGTTC |
| | | CAGGCCCGCTGCCGCGCCTATCTGGTGCGCAAGGCCTTCCGCCACCGCCTCTGGGCTGTG |
| | | CTCACCGTGCAGGCCTATGCCCGGGGCATGATCGCCCGCAGGCTGCACCAACGCCTCAGG |
| | | GCTGAGTATCTGTGGCGCCTCGAGGCTGAGAAAATGCGGCTGGCGGAGGAAGAGAAGCTT |
| | | CGGAAGGAGATGAGCGCCAAGAAGGCCAAGGAGGAGGCCGAGCGCAAGCATCAGGAGCGC |
| | | CTGGCCCAGCTGGCTCGTGAGGACGCTGAGCGGGAGCTGAAGGAGAAGGAGGCCGCTCGG |
| | | CGGAAGAAGGAGCTCCTGGAGCAGATGGAAAGGGCCCGCCATGAGCCTGTCAATCACTCA |
| | | GACATGGTGGACAAGATGTTTGGCTTCCTGGGGACTTCAGGTGGCCTGCCAGGCCAGGAG |
| | | GGCCAGGCACCTAGTGGCTTTGAGGACCTGGAGCGAGGGCGGAGGGAGATGGTGGAGGAG |
| | | GACCTGGATGCAGCCCTGCCCCTGCCTGACGAGGATGAGGAGGACCTCTCTGAGTATAAA |
| | | TTTGCCAAGTTCGCGGCCACCTACTTCCAGGGGACAACCACGCACTCCTACACCCGGCGG |
| | | CCACTCAAACAGCCACTGCTCTACCATGACGACGAGGGTGACCAGCTGGCAGCCCTGGCG |
| | | GTCTGGATCACCATCCTCCGCTTCATGGGGGACCTCCCTGAGCCCAAGTACCACACAGCC |
| | | ATGAGTGATGGCAGTGAGAAGATCCCTGTGATGACCAAGATTTATGAGACCCTGGGCAAG |
| | | AAGACGTACAAGAGGGAGCTGCAGGCCCTGCAGGGCGAGGGCGAGGCCCAGCTCCCCGAG |
| | | GGCCAGAAGAAGAGCAGTGTGAGGCACAAGCTGGTGCATTTGACTCTGAAAAAGAAGTCC |
| | | AAGCTCACAGAGGAGGTGACCAAGAGGCTGCATGACGGGGAGTCCACAGTGCAGGGCAAC |
| | | AGCATGCTGGAGGACCGGCCCACCTCCAACCTGGAGAAGCTGCACTTCATCATCGGCAAT |
| | | GGCATCCTGCGGCCAGCACTCCGGGACGAGATCTACTGCCAGATCAGCAAGCAGCTGACC |
| | | CACAACCCCTCCAAGAGCAGCTATGCCCGGGGCTGGATTCTCGTGTCTCTCTGCGTGGGC |
| | | TGTTTCGCCCCCTCCGAGAAGTTTGTCAAGTACCTGCGGAACTTCATCCACGGGGGCCCG |
| | | CCCGGCTACGCCCCGTACTGTGAGGAGCGCCTGAGAAGGACCTTTGTCAATGGGACACGG |
| | | ACACAGCCGCCCAGCTGGCTGGAGCTGCAGGCCACCAAGTCCAAGAAGCCAATCATGTTG |
| | | CCCGTGACATTCATGGATGGGACCACCAAGACCCTGCTGACGGACTCGGCAACCACGGCC |
| | | AAGGAGCTCTGCAACGCGCTGGCCGACAAGATCTCTCTCAAGGACCGGTTCGGGTTCTCC |
| | | CTCTACATTGCCCTGTTTGACAAGGTGTCCTCCCTGGGCAGCGGCAGTGACCACGTCATG |
| | | GACGCCATCTCCCAGTGCGAGCAGTACGCCAAGGAGCAGGGCGCCCAGGAGCGCAACGCC |
| | | CCCTGGAGGCTCTTCTTCCGCAAAGAGGTCTTCACGCCCTGGCACAGCCCCTCCGAGGAC |
| | | AACGTGGCCACCAACCTCATCTACCAGCAGGTGGTGCGAGGAGTCAAGTTTGGGGAGTAC |
| | | AGGTGTGAGAAGGAGGACGACCTGGCTGAGCTGGCCTCCCAGCAGTACTTTGTAGACTAT |
| | | GGCTCTGAGATGATCCTGGAGCGCCTCCTGAACCTCGTGCCCACCTACATCCCCGACCGC |
| | | GAGATCACGCCCCTGAAGACGCTGGAGAAGTGGGCCCAGCTGGCCATCGCCGCCCACAAG |
| | | AAGGGGATTTATGCCCAGAGGAGAACTGATGCCCAGAAGGTCAAAGAGGATGTGGTCAGT |
| | | TATGCCCGCTTCAAGTGGCCCTTGCTCTTCTCCAGGTTTTATGAAGCCTACAAATTCTCA |
| | | GGCCCCAGTCTCCCCAAGAACGACGTCATCGTGGCCGTCAACTGGACGGGTGTGTACTTT |
| | | GTGGATGAGCAGGAGCAGGTACTTCTGGAGCTGTCCTTCCCAGAGATCATGGCCGTGTCC |
| | | AGCAGCAGGGAGTGCCGTGTCTGGCTCTCACTGGGCTGCTCTGATCTTGGCTGTGCTGCG |
| | | CCTCACTCAGGCTGGGCAGGACTGACCCCGGCGGGGCCCTGTTCTCCGTGTTGGTCCTGC |
| | | AGGGGAGCGAAAACGACGGCCCCCAGCTTCACGCTGGCCACCATCAAGGGGGACGAATAC |
| | | ACCTTCACCTCCAGTAATGCTGAGGACATTCGTGACCTGGTGGTCACCTTCCTAGAGGGG |
| | | CTCCGGAAGAGATCTAAGTATGTTGTGGCCCTGCAGGATAACCCCAACCCCGCAGGCGAG |
| | | GAGTCAGGCTTCCTCAGCTTTGCCAAGGGAGACCTCATCATCCTGGACCATGACACGGGC |
| | | GAGCAGGTCATGAACTCGGGCTGGGCCAACGGCATCAATGAGAGGACCAAGCAGCGTGGG |
| | | GACTTCCCCACCGACTGTGTGTACGTCATGCCCACTGTCACCATGCCACCGCGGGAGATT |
| | | GTGGCCCTGGTCACCATGACTCCCGATCAGAGGCAGGACGTTGTCCGGCTCTTGCAGCTG |
| | | CGAACGGCGGAGCCCGAGGTGCGTGCCAAGCCCTACACGCTGGAGGAGTTTTCCTATGAC |
| | | TACTTCAGGCCCCCACCCAAGCACACGCTGAGCCGTGTCATGGTGTCCAAGGCCCGAGGC |
| | | AAGGACCGGCTGTGGAGCCACACGCGGGAACCGCTCAAGCAGGCGCTGCTCAAGAAGCTC |
| | | CTGGGCAGTGAGGAGCTCTCGCAGGAGGCCTGCCTGGCCTTCATTGCTGTGCTCAAGTAC |
| | | ATGGGCGACTACCCGTCCAAGAGGACACGCTCCGTCAACGAGCTCACCGACCAGATCTTT |
| | | GAGGGTCCCCTGAAAGCCGAGCCCCTGAAGGACGAGGCATATGTGCAGATCCTGAAGCAG |
| | | CTGACCGACAACCACATCAGGTACAGCGAGGAGCGGGGTTGGGAGCTGCTCTGGCTGTGC |
| | | ACGGGCCTTTTCCCACCCAGCAACATCCTCCTGCCCCACGTGCAGCGCTTCCTGCAGTCC |
| | | CGAAAGCACTGCCCACTCGCCATCGACTGCCTGCAACGGCTCCAGAAAGCCCTGAGAAAC |
| | | GGGTCCCGGAAGTACCCTCCGCACCTGGTGGAGGTGGAGGCCATCCAGCACAAGACCACC |
| | | CAGATTTTCCACAAGGTCTACTTCCCTGATGACACTGACGAGGCCTTCGAAGTGGAGTCC |
| | | AGCACCAAGGCCAAGGACTTCTGCCAGAACATCGCCACCAGGCTGCTCCTCAAGTCCTCA |
| | | GAGGGATTCAGCCTCTTTGTCAAAATTGCAGACAAGGTCATCAGCGTTCCTGAGAATGAC |
| | | TTCTTCTTTGACTTTGTTCGACACTTGACAGACTGGATAAAGAAAGCTCGGCCCATCAAG |
| | | GACGGAATTGTGCCCTCACTCACCTACCAGGTGTTCTTCATGAAGAAGCTGTGGACCACC |
| | | ACGGTGCCAGGGAAGGATCCCATGGCCGATTCCATCTTCCACTATTACCAGGAGTTGCCC |
| | | AAGTATCTCCGAGGCTACCACAAGTGCACGCGGGAGGAGGTGCTGCAGCTGGGGGCGCTG |
| | | ATCTACAGGGTCAAGTTCGAGGAGGACAAGTCCTACTTCCCCAGCATCCCCAAGCTGCTG |
| | | CGGGAGCTGGTGCCCCAGGACCTTATCCGGCAGGTCTCACCTGATGACTGGAAGCGGTCC |
| | | ATCGTCGCCTACTTCAACAAGCACGCAGGGAAGTCCAAGGAGGAGGCCAAGCTGGCCTTC |
| | | CTGAAGCTCATCTTCAAGTGGCCCACCTTTGGCTCAGCCTTCTTCGAGGTGAAGCAAACT |
| | | ACGGAGCCAAACTTCCCTGAGATCCTCCTAATTGCCATCAACAAGTATGGGGTCAGCCTC |
| | | ATCGATCCCAAAACGAAGGATATCCTCACCACTCATCCCTTCACCAAGATCTCCAACTGG |
| | | AGCAGCGGCAACACCTACTTCCACATCACCATTGGGAACTTGGTGCGCGGGAGCAAACTG |
| | | CTCTGCGAGACGTCACTGGGCTACAAGATGGATGACCTCCTGACTTCCTACATTAGCCAG |
| | | ATGCTCACAGCCATGAGCAAACAGCGGGGCTCCAGGAGCGGCAAGTGA |
| 2 | MYO7A | MVILQQGDHVWMDLRLGQEFDVPIGAVVKLCDSGQVQVVDDEDNEHWISPQNATHIKPMH |
| | isoform | PTSVHGVEDMIRLGDLNEAGILRNLLIRYRDHLIYTYTGSILVAVNPYQLLSIYSPEHIR |
| | 1 | QYTNKKIGEMPPHIFAIADNCYFNMKRNSRDQCCIISGESGAGKTESTKLILQFLAAISG |
| | protein | QHSWIEQQVLEATPILEAFGNAKTIRNDNSSRFGKYIDIHFNKRGAIEGAKIEQYLLEKS |
| | | RVCRQALDERNYHVFYCMLEGMSEDQKKKLGLGQASDYNYLAMGNCITCEGRVDSQEYAN |
| | | IRSAMKVLMFTDTENWEISKLLAAILHLGNLQYEARTFENLDACEVLFSPSLATAASLLE |
| | | VNPPDLMSCLTSRTLITRGETVSTPLSREQALDVRDAFVKGIYGRLFVWIVDKINAAIYK |
| | | PPSQDVKNSRRSIGLLDIFGFENFAVNSFEQLCINFANEHLQQFFVRHVFKLEQEEYDLE |
| | | SIDWLHIEFTDNQDALDMIANKPMNIISLIDEESKFPKGTDTTMLHKLNSQHKLNANYIP |
| | | PKNNHETQFGINHFAGIVYYETQGFLEKNRDTLHGDIIQLVHSSRNKFIKQIFQADVAMG |
| | | AETRKRSPTLSSQFKRSLELLMRTLGACQPFFVRCIKPNEFKKPMLFDRHLCVRQLRYSG |
| | | MMETIRIRRAGYPIRYSFVEFVERYRVLLPGVKPAYKQGDLRGTCQRMAEAVLGTHDDWQ |
| | | IGKTKIFLKDHHDMLLEVERDKAITDRVILLQKVIRGFKDRSNFLKLKNAATLIQRHWRG |
| | | HNCRKNYGLMRLGFLRLQALHRSRKLHQQYRLARQRIIQFQARCRAYLVRKAFRHRLWAV |
| | | LTVQAYARGMIARRLHQRLRAEYLWRLEAEKMRLAEEEKLRKEMSAKKAKEEAERKHQER |
| | | LAQLAREDAERELKEKEAARRKKELLEQMERARHEPVNHSDMVDKMFGFLGTSGGLPGQE |
| | | GQAPSGFEDLERGRREMVEEDLDAALPLPDEDEEDLSEYKFAKFAATYFQGTTTHSYTRR |
| | | PLKQPLLYHDDEGDQLAALAVWITILRFMGDLPEPKYHTAMSDGSEKIPVMTKIYETLGK |
| | | KTYKRELQALQGEGEAQLPEGQKKSSVRHKLVHLTLKKKSKLTEEVTKRLHDGESTVQGN |
| | | SMLEDRPTSNLEKLHFIIGNGILRPALRDEIYCQISKQLTHNPSKSSYARGWILVSLCVG |
| | | CFAPSEKFVKYLRNFIHGGPPGYAPYCEERLRRTFVNGTRTQPPSWLELQATKSKKPIML |
| | | PVTFMDGTTKTLLTDSATTAKELCNALADKISLKDRFGFSLYIALFDKVSSLGSGSDHVM |
| | | DAISQCEQYAKEQGAQERNAPWRLFFRKEVFTPWHSPSEDNVATNLIYQQVVRGVKFGEY |
| | | RCEKEDDLAELASQQYFVDYGSEMILERLLNLVPTYIPDREITPLKTLEKWAQLAIAAHK |
| | | KGIYAQRRTDAQKVKEDVVSYARFKWPLLFSRFYEAYKFSGPSLPKNDVIVAVNWTGVYF |
| | | VDEQEQVLLELSFPEIMAVSSSRECRVWLSLGCSDLGCAAPHSGWAGLTPAGPCSPCWSC |
| | | RGAKTTAPSFTLATIKGDEYTFTSSNAEDIRDLVVTFLEGLRKRSKYVVALQDNPNPAGE |
| | | ESGFLSFAKGDLIILDHDTGEQVMNSGWANGINERTKQRGDFPTDSVYVMPTVTMPPREI |
| | | VALVTMTPDQRQDVVRLLQLRTAEPEVRAKPYTLEEFSYDYFRPPPKHTLSRVMVSKARG |
| | | KDRLWSHTREPLKQALLKKLLGSEELSQEACLAFIAVLKYMGDYPSKRTRSVNELTDQIF |
| | | EGPLKAEPLKDEAYVQILKQLTDNHIRYSEERGWELLWLCTGLFPPSNILLPHVQRFLQS |
| | | RKHCPLAIDCLQRLQKALRNGSRKYPPHLVEVEAIQHKTTQIFHKVYFPDDTDEAFEVES |
| | | STKAKDFCQNIATRLLLKSSEGFSLFVKIADKVLSVPENDFFFDFVRHLTDWIKKARPIK |
| | | DGIVPSLTYQVFFMKKLWTTTVPGKDPMADSIFHYYQELPKYLRGYHKCTREEVLQLGAL |
| | | IYRVKFEEDKSYFPSIPKLLRELVPQDLIRQVSPDDWKRSIVAYFNKHAGKSKEEAKLAF |
| | | LKLIFKWPTFGSAFFEVKQTTEPNFPEILLIAINKYGVSLIDPKTKDILTTHPFTKISNW |
| | | SSGNTYFHITIGNLVRGSKLLCETSLGYKMDDLLTSYISQMLTAMSKQRGSRSGK |
| 3 | MYO7A | MVILQQGDHVWMDLRLGQEFDVPIGAVVKLCDSGQVQVVDDEDNEHWISPQNATHIKPMH |
| | isoform | PTSVHGVEDMIRLGDLNEAGILRNLLIRYRDHLIYTYTGSILVAVNPYQLLSIYSPEHIR |
| | 2 | QYTNKKIGEMPPHIFAIADNCYFNMKRNSRDQCCIISGESGAGKTESTKLILQFLAAISG |
| | protein | QHSWIEQQVLEATPILEAFGNAKTIRNDNSSRFGKYIDIHFNKRGAIEGAKIEQYLLEKS |
| | | RVCRQALDERNYHVFYCMLEGMSEDQKKKLGLGQASDYNYLAMGNCITCEGRVDSQEYAN |
| | | IRSAMKVLMFTDTENWEISKLLAAILHLGNLQYEARTFENLDACEVLFSPSLATAASLLE |
| | | VNPPDLMSCLTSRTLITRGETVSTPLSREQALDVRDAFVKGIYGRLFVWIVDKINAAIYK |
| | | PPSQDVKNSRRSIGLLDIFGFENFAVNSFEQLCINFANEHLQQFFVRHVFKLEQEEYDLE |
| | | SIDWLHIEFTDNQDALDMIANKPMNIISLIDEESKFPKGTDTTMLHKLNSQHKLNANYIP |
| | | PKNNHETQFGINHFAGIVYYETQGFLEKNRDTLHGDIIQLVHSSRNKFIKQIFQADVAMG |
| | | AETRKRSPTLSSQFKRSLELLMRTLGACQPFFVRCIKPNEFKKPMLFDRHLCVRQLRYSG |
| | | MMETIRIRRAGYPIRYSFVEFVERYRVLLPGVKPAYKQGDLRGTCQRMAEAVLGTHDDWQ |
| | | IGKTKIFLKDHHDMLLEVERDKAITDRVILLQKVIRGFKDRSNFLKLKNAATLIQRHWRG |
| | | HNCRKNYGLMRLGFLRLQALHRSRKLHQQYRLARQRIIQFQARCRAYLVRKAFRHRLWAV |
| | | LTVQAYARGMIARRLHQRLRAEYLWRLEAEKMRLAEEEKLRKEMSAKKAKEEAERKHQER |
| | | LAQLAREDAERELKEKEAARRKKELLEQMERARHEPVNHSDMVDKMFGFLGTSGGLPGQE |
| | | GQAPSGFEDLERGRREMVEEDLDAALPLPDEDEEDLSEYKFAKFAATYFQGTTTHSYTRR |
| | | PLKQPLLYHDDEGDQLAALAVWITILRFMGDLPEPKYHTAMSDGSEKIPVMTKIYETLGK |
| | | KTYKRELQALQGEGEAQLPEGQKKSSVRHKLVHLTLKKKSKLTEEVTKRLHDGESTVQGN |
| | | SMLEDRPTSNLEKLHFIIGNGILRPALRDEIYCQISKQLTHNPSKSSYARGWILVSLCVG |
| | | CFAPSEKFVKYLRNFIHGGPPGYAPYCEERLRRTFVNGTRTQPPSWLELQATKSKKPIML |
| | | PVTFMDGITKILLTDSATTAKELCNALADKISLKDRFGFSLYIALFDKVSSLGSGSDHVM |
| | | DAISQCEQYAKEQGAQERNAPWRLFFRKEVFTPWHSPSEDNVATNLIYQQVVRGVKFGEY |
| | | RCEKEDDLAELASQQYFVDYGSEMILERLLNLVPTYIPDREITPLKTLEKWAQLAIAAHK |
| | | KGIYAQRRTDAQKVKEDVVSYARFKWPLLFSRFYEAYKFSGPSLPKNDVIVAVNWTGVYF |
| | | VDEQEQVLLELSFPEIMAVSSSRGAKTTAPSFTLATIKGDEYTFTSSNAEDIRDLVVTFL |
| | | EGLRKRSKYVVALQDNPNPAGEESGFLSFAKGDLIILDHDTGEQVMNSGWANGINERTKQ |
| | | RGDFPTDSVYVMPTVTMPPREIVALVTMTPDQRQDVVRLLQLRTAEPEVRAKPYTLEEFS |
| | | YDYFRPPPKHTLSRVMVSKARGKDRLWSHTREPLKQALLKKLLGSEELSQEACLAFIAVL |
| | | KYMGDYPSKRTRSVNELTDQIFEGPLKAEPLKDEAYVQILKQLTDNHIRYSEERGWELLW |
| | | LCTGLFPPSNILLPHVQRFLQSRKHCPLAIDCLQRLQKALRNGSRKYPPHLVEVEAIQHK |
| | | TTQIFHKVYFPDDTDEAFEVESSTKAKDFCQNIATRLLLKSSEGFSLFVKIADKVLSVPE |
| | | NDFFFDFVRHLTDWIKKARPIKDGIVPSLTYQVFFMKKLWITTVPGKDPMADSIFHYYQE |
| | | LPKYLRGYHKCTREEVLQLGALIYRVKFEEDKSYFPSIPKLLRELVPQDLIRQVSPDDWK |
| | | RSIVAYFNKHAGKSKEEAKLAFLKLIFKWPTFGSAFFEQTTEPNFPEILLIAINKYGVSL |
| | | IDPKTKDILTTHPFTKISNWSSGNTYFHITIGNLVRGSKLLCETSLGYKMDDLLTSYISQ |
| | | MLTAMSKQRGSRSGK |
| 4 | MYO7A | MVILQQGDHVWMDLRLGQEFDVPIGAVVKLCDSGQVQVVDDEDNEHWISPQNATHIKPMH |
| | isoform | PTSVHGVEDMIRLGDLNEAGILRNLLIRYRDHLIYTYTGSILVAVNPYQLLSIYSPEHIR |
| | 3 | QYTNKKIGEMPPHIFAIADNCYFNMKRNSRDQCCIISGESGAGKTESTKLILQFLAAISG |
| | protein | QKSWIEQQVLEATPILEAFGNAKTIRNDNSSRFGKYIDIHFNKRGAIEGAKIEQYLLEKS |
| | | RVCRQALDERNYHVFYCMLEGMSEDQKKKLGLGQASDYNYLAMGNCITCEGRVDSQEYAN |
| | | IRSAMKVLMFTDTENWEISKLLAAILHLGNLQYEARTFENLDACEVLFSPSLATAASLLE |
| | | VNPPDLMSCLTSRTLITRGETVSTPLSREQALDVRDAFVKGIYGRLFVWIVDKINAAIYK |
| | | PPSQDVKNSRRSIGLLDIFGFENFAVNSFEQLCINFANEHLQQFFVRHVFKLEQEEYDLE |
| | | SIDWLHIEFTDNQDALDMIANKPMNIISLIDEESKFPKGTDTTMLHKLNSQHKLNANYIP |
| | | PKNNHETQFGINHFAGIVYYETQGFLEKNRDTLHGDIIQLVHSSRNKFIKQIFQADVAMG |
| | | AETRKRSPTLSSQFKRSLELLMRTLGACQPFFVRCIKPNEFKKPMLFDRHLCVRQLRYSG |
| | | MMETIRIRRAGYPIRYSFVEFVERYRVLLPGVKPAYKQGDLRGTCQRMAEAVLGTHDDWQ |
| | | IGKTKIFLKDHHDMLLEVERDKAITDRVILLQKVIRGFKDRSNFLKLKNAATLIQRHWRG |
| | | HNCRKNYGLMRLGFLRLQALHRSRKLHQQYRLARQRIIQFQARCRAYLVRKAFRHRLWAV |
| | | LTVQAYARGMIARRLHQRLRAEYLWRLEAEKMRLAEEEKLRKEMSAKKAKEEAERKHQER |
| | | LAQLAREDAERELKEKEAARRKKELLEQMERARHEPVNHSDMVDKMFGFLGTSGGLPGQE |
| | | GQAPSGFEDLERGRREMVEEDLDAALPLPDEDEEDLSEYKFAKFAATYFQGTTTHSYTRR |
| | | PLKQPLLYHDDEGDQLAALAVWITILRFMGDLPEPKYHTAMSDGSEKIPVMTKIYETLGK |
| | | KTYKRELQALQGEGEAQLPEGQKKSSVRHKLVHLTLKKKSKLTEEVTKRLHDGESTVQGN |
| | | SMLEDRPTSNLEKLHFIIGNGILRPALRSVPESLLVAEWCLCQPSKRLSQAWPGFGFAAS |
| 5 | MYO7A | MVILQQGDHVWMDLRLGQEFDVPIGAVVKLCDSGQVQVVDDEDNEHWISPQNATHIKPMH |
| | isoform | PTSVHGVEDMIRLGDLNEAGILRNLLIRYRDHLIYTYTGSILVAVNPYQLLSIYSPEHIR |
| | 4 | QYTNKKIGEMPPHIFAIADNCYFNMKRNSRDQCCIISGESGAGKTESTKLILQFLAAISG |
| | protein | QHSWIEQQVLEATPILEAFGNAKTIRNDNSSRFGKYIDIHFNKRGAIEGAKIEQYLLEKS |
| | | RVCRQALDERNYHVFYCMLEGMSEDQKKKLGLGQASDYNYLAMGNCITCEGRVDSQEYAN |
| | | IRSAMKVLMFTDTENWEISKLLAAILHLGNLQYEARTFENLDACEVLFSPSLATAASLLE |
| | | VNPPDLMSCLTSRTLITRGETVSTPLSREQALDVRDAFVKGIYGRLFVWIVDKINAAIYK |
| | | PPSQDVKNSRRSIGLLDIFGFENFAVNSFEQLCINFANEHLQQFFVRHVFKLEQEEYDLE |
| | | SIDWLHIEFTDNQDALDMIANKPMNIISLIDEESKFPKGTDTTMLHKLNSQHKLNANYIP |
| | | PKNNHETQFGINHFAGIVYYETQGFLEKNRDTLHGDIIQLVHSSRNKFIKQIFQADVAMG |
| | | AETRKRSPTLSSQFKRSLELLMRTLGACQPFFVRCIKPNEFKKPMLFDRHLCVRQLRYSG |
| | | MMETIRIRRAGYPIRYSFVEFVERYRVLLPGVKPAYKQGDLRGTCQRMAEAVLGTHDDWQ |
| | | IGKTKIFLKDHHDMLLEVERDKAITDRVILLQKVIRGFKDRSNFLKLKNAATLIQRHWRG |
| | | HNCRKNYGLMRLGFLRLQALHRSRKLHQQYRLARQRIIQFQARCRAYLVRKAFRHRLWAV |
| | | LTVQAYARGMIARRLHQRLRAEYLWRLEAEKMRLAEEEKLRKEMSAKKAKEEAERKHQER |
| | | LAQLAREDAERELKEKEAARRKKELLEQMERARHEPVNHSDMVDKMFGFLGTSGGLPGQE |
| | | GQAPSGFEDLERGRREMVEEDLDAALPLPDEDEEDLSEYKFAKFAATYFQGTTTHSYTRR |
| | | PLKQPLLYHDDEGDQLAALAVWITILRFMGDLPEPKYHTAMSDGSEKIPVMTKIYETLGK |
| | | KTYKRELQALQGEGEVLQAQLPEGQKKSSVRHKLVHLTLKKKSKLTEEVTKRLHDGESTV |
| | | QGNSMLEDRPTSNLEKLHFIIGNGILRPALRSVPESLLVAEWCLCQPSKRLSQAWPGFGF |
| | | AAS |
| 6 | MYO7A | MVILQQGDHVWMDLRLGQEFDVPIGAVVKLCDSGQVQVVDDEDNEHWISPQNATHIKPMH |
| | isoform | PTSVHGVEDMIRLGDLNEAGILRNLLIRYRDHLIYTYTGSILVAVNPYQLLSIYSPEHIR |
| | 5 | QYTNKKIGEMPPHIFAIADNCYFNMKRNSRDQCCIISGESGAGKTESTKLILQFLAAISG |
| | protein | QKSWIEQQVLEATPILEAFGNAKTIRNDNSSRFGKYIDIHFNKRGAIEGAKIEQYLLEKS |
| | | RVCRQALDERNYHVFYCMLEGMSEDQKKKLGLGQASDYNYLAMVNPPDLMSCLTSRTLIT |
| | | RGETVSTPLSREQALDVRDAFVKGIYGRLFVNIVDKINAAIYKPPSQDVKNSRRSIGLLD |
| | | IFGFENFAVNSFEQLCINFANEHLQQFFVRHVFKLEQEEYDLESIDWLHIEFTDNQDALD |
| | | MIANKPMNIISLIDEESKFPKFLEKNRDTLHGDIIQLVHSSRNKFIKQIFQADVAMGAET |
| | | RKRSPTLSSQFKRSLELLMRTLGACQPFFVRCIKPNEFKKPMLFDRHLCVRQLRYSGMME |
| | | TIRIRRAGYPIRYSFVEFVERYRVLLPGVKPAYKQGDLRGTCQRMAEAVLGTHDDWQIGK |
| | | TKIFLKDHHDMLLEVERDKAITDRVILLQKVIRGFKDRSNFLKLKNAATLIQRHWRGHNC |
| | | RKNYGLMRLGFLRLQALHRSRKLHQQYRLARQRIIQFQARCRAYLVRKAFRHRLWAVLTV |
| | | QAYARGMIARRLHQRLRAEYLWRLEAEKMRLAEEEKLRKEMSAKKAKEEAERKHQERLAQ |
| | | LAREDAERELKEKEAARRKKELLEQMERARHEPVNHSDMVDKMFGFLGTSGGLPGQEGQA |
| | | PSGFEDLERGRREMVEEDLDAALPLPDEDEEDLSEYKFAKFAATYFQGTTTHSYTRRPLK |
| | | QPLLYHDDEGDQLAALAVWITILRFMGDLPEPKYHTAMSDGSEKIPVMTKIYETLGKKTY |
| | | KRELQALQGEGEAQLPEGQKKSSVRHKLVHLTLKKKSKLTEEVTKRLHDGESTVQGNSML |
| | | EDRPTSNLEKLHFIIGNGILRPALRDEIYCQISKQLTHNPSKSSYARGWILVSLCVGCFA |
| | | PSEKFVKYLRNFIHGGPPGYAPYCEERLRRTFVNGTRTQPPSWLELQATKSKKPIMLPVT |
| | | FMDGITKILLTDSATTAKELCNALADKISLKDRFGFSLYIALFDKVSSLGSGSDHVMDAI |
| | | SQCEQYAKEQGAQERNAPWRLFFRKEVFTPWHSPSEDNVATNLIYQQVVRGVKFGEYRCE |
| | | KEDDLAELASQQYFVDYGSEMILERLLNLVPTYIPDREITPLKTLEKWAQLAIAAHKKGI |
| | | YAQRRTDAQKVKEDVVSYARFKWPLLFSRFYEAYKFSGPSLPKNDVIVAVNWTGVYFVDE |
| | | QEQVLLELSFPEIMAVSSSRECRVWLSLGCSDLGCAAPHSGWAGLTPAGPCSPCWSCRGA |
| | | KTTAPSFTLATIKGDEYTFTSSNAEDIRDLVVTFLEGLRKRSKYVVALQDNPNPAGEESG |
| | | FLSFAKGDLIILDHDTGEQVMNSGWANGINERTKQRGDFPTDSVYVMPTVIMPPREIVAL |
| | | VTMTPDQRQDVVRLLQLRTAEPEVRAKPYTLEEFSYDYFRPPPKHTLSRVMVSKARGKDR |
| | | LWSHTREPLKQALLKKLLGSEELSQEACLAFIAVLKYMGDYPSKRTRSVNELTDQIFEGP |
| | | LKAEPLKDEAYVQILKQLTDNHIRYSEERGWELLWLCTGLFPPSNILLPHVQRFLQSRKH |
| | | CPLAIDCLQRLQKALRNGSRKYPPHLVEVEAIQHKTTQIFHKVYFPDDTDEAFEVESSTK |
| | | AKDFCQNIATRLLLKSSEGFSLFVKIADKVLSVPENDFFFDFVRHLTDWIKKARPIKDGI |
| | | VPSLTYQVFFMKKLWTTTVPGKDPMADSIFHYYQELPKYLRGYHKCTREEVLQLGALIYR |
| | | VKFEEDKSYFPSIPKLLRELVPQDLIRQVSPDDWKRSIVAYFNKHAGKSKEEAKLAFLKL |
| | | IFKWPTFGSAFFEVKQTTEPNFPEILLIAINKYGVSLIDPKTKDILTTHPFTKISNWSSG |
| | | NTYFHITIGNLVRGSKLLCETSLGYKMDDLLTSYISQMLTAMSKQRGSRSGK |
| 7 | MYO7A | MVILQQGDHVWMDLRLGQEFDVPIGAVVKLCDSGQVQVVDDEDNEHWISPQNATHIKPMH |
| | isoform | PTSVHGVEDMIRLGDLNEAGILRNLLIRYRDHLIYTYTGSILVAVNPYQLLSIYSPEHIR |
| | 6 | QYTNKKIGEMPPHIFAIADNCYFNMKRNSRDQCCIISGESGAGKTESTKLILQFLAAISG |
| | protein | QHSWIEQQVLEATPILEAFGNAKTIRNDNSSRFGKYIDIHFNKRGAIEGAKIEQYLLEKS |
| | | RVCRQALDERNYHVFYCMLEGMSEDQKKKLGLGQASDYNYLAMGNCITCEGRVDSQEYAN |
| | | IRSAMKVLMFTDTENWEISKLLAAILHLGNLQYEARTFENLDACEVLFSPSLATAASLLE |
| | | VNPPDLMSCLTSRTLITRGETVSTPLSREQALDVRDAFVKGIYGRLFVWIVDKINAAIYK |
| | | PPSQDVKNSRRSIGLLDIFGFENFAVNSFEQLCINFANEHLQQFFVRHVFKLEQEEYDLE |
| | | SIDWLHIEFTDNQDALDMIANKPMNIISLIDEESKFPKGTDTTMLHKLNSQHKLNANYIP |
| | | PKNNHETQFGINHFAGIVYYETQGFLEKNRDTLHGDIIQLVHSSRNKFIKQIFQADVAMG |
| | | AETRKRSPTLSSQFKRSLELLMRTLGACQPFFVRCIKPNEFKKPMLFDRHLCVRQLRYSG |
| | | MMETIRIRRAGYPIRYSFVEFVERYRVLLPGVKPAYKQGDLRGTCQRMAEAVLGTHDDWQ |
| | | IGKTKIFLKDHHDMLLEVERDKAITDRVILLQKVIRGFKDRSNFLKLKNAATLIQRHWRG |
| | | HNCRKNYGLMRLGFLRLQALHRSRKLHQQYRLARQRIIQFQARCRAYLVRKAFRHRLWAV |
| | | LTVQAYARGMIARRLHQRLRAEYLWRLEAEKMRLAEEEKLRKEMSAKKAKEEAERKHQER |
| | | LAQLAREDAERELKEKEAARRKKELLEQMERARHEPVNHSDMVDKMFGFLGTSGGLPGQE |
| | | GQAPSGFEDLERGRREMVEEDLDAALPLPDEDEEDLSEYKFAKFAATYFQGTTTHSYTRR |
| | | PLKQPLLYHDDEGDQLAALAVWITILRFMGDLPEPKYHTAMSDGSEKIPVMTKIYETLGK |
| | | KTYKRELQALQGEGEVTKRLHDGESTVQGNSMLEDRPTSNLEKLHFIIGNGILRPALRDE |
| | | IYCQISKQLTHNPSKSSYARGWILVSLCVGCFAPSEKFVKYLRNFIHGGPPGYAPYCEER |
| | | LRRTFVNGTRTQPPSWLELQATKSKKPIMLPVTFMDGITKILLTDSATTAKELCNALADK |
| | | ISLKDRFGFSLYIALFDKVSSLGSGSDHVMDAISQCEQYAKEQGAQERNAPWRLFFRKEV |
| | | FTPWHSPSEDNVATNLIYQQVVRGVKFGEYRCEKEDDLAELASQQYFVDYGSEMILERLL |
| | | NLVPTYIPDREITPLKTLEKWAQLAIAAHKKGIYAQRRTDAQKVKEDVVSYARFKWPLLF |
| | | SRFYEAYKFSGPSLPKNDVIVAVNWTGVYFVDEQEQVLLELSFPEIMAVSSSRECRVWLS |
| | | LGCSDLGCAAPHSGWAGLTPAGPCSPCWSCRGAKTTAPSFTLATIKGDEYTFTSSNAEDI |
| | | RDLVVTFLEGLRKRSKYVVALQDNPNPAGEESGFLSFAKGDLIILDHDTGEQVMNSGWAN |
| | | GINERTKQRGDFPTDSVYVMPTVTMPPREIVALVTMTPDQRQDVVRLLQLRTAEPEVRAK |
| | | PYTLEEFSYDYFRPPPKHTLSRVMVSKARGKDRLWSHTREPLKQALLKKLLGSEELSQEA |
| | | CLAFIAVLKYMGDYPSKRTRSVNELTDQIFEGPLKAEPLKDEAYVQILKQLTDNHIRYSE |
| | | ERGWELLWLCTGLFPPSNILLPHVQRFLQSRKHCPLAIDCLQRLQKALRNGSRKYPPHLV |
| | | EVEAIQHKTTQIFHKVYFPDDTDEAFEVESSTKAKDFCQNIATRLLLKSSEGFSLFVKIA |
| | | DKVLSVPENDFFFDFVRHLTDWIKKARPIKDGIVPSLTYQVFFMKKLWITTVPGKDPMAD |
| | | SIFHYYQELPKYLRGYHKCTREEVLQLGALIYRVKFEEDKSYFPSIPKLLRELVPQDLIR |
| | | QVSPDDWKRSIVAYFNKHAGKSKEEAKLAFLKLIFKWPTFGSAFFEVKQTTEPNFPEILL |
| | | IAINKYGVSLIDPKTKDILTTHPFTKISNWSSGNTYFHITIGNLVRGSKLLCETSLGYKM |
| | | DDLLTSYISQMLTAMSKQRGSRSGK |
| 8 | MYO7A | MVILQQGDHVWMDLRLGQEFDVPIGAVVKLCDSGQVQVVDDEDNEHWISPQNATHIKPMH |
| | isoform | PTSVHGVEDMIRLGDLNEAGILRNLLIRYRDHLIYTYTGSILVAVNPYQLLSIYSPEHIR |
| | 7 | QYTNKKIGEMPPHIFAIADNCYFNMKRNSRDQCCIISGESGAGKTESTKLILQFLAAISG |
| | protein | QHSWIEQQVLEATPILEAFGNAKTIRNDNSSRFGKYIDIHFNKRGAIEGAKIEQYLLEKS |
| | | RVCRQALDERNYHVFYCMLEGMSEDQKKKLGLGQASDYNYLAMGNCITCEGRVDSQEYAN |
| | | IRSAMKVLMFTDTENWEISKLLAAILHLGNLQYEARTFENLDACEVLFSPSLATAASLLE |
| | | VNPPDLMSCLTSRTLITRGETVSTPLSREQALDVRDAFVKGIYGRLFVWIVDKINAAIYK |
| | | PPSQDVKNSRRSIGLLDIFGFENFAVNSFEQLCINFANEHLQQFFVRHVFKLEQEEYDLE |
| | | SIDWLHIEFTDNQDALDMIANKPMNIISLIDEESKFPKGTDTTMLHKLNSQHKLNANYIP |
| | | PKNNHETQFGINHFAGIVYYETQGFLEKNRDTLHGDIIQLVHSSRNKFIKQIFQADVAMG |
| | | AETRKRSPTLSSQFKRSLELLMRTLGACQPFFVRCIKPNEFKKPMLFDRHLCVRQLRYSG |
| | | MMETIRIRRAGYPIRYSFVEFVERYRVLLPGVKPAYKQGDLRGTCQRMAEAVLGTHDDWQ |
| | | IGKTKIFLKDHHDMLLEVERDKAITDRVILLQKVIRGFKDRSNFLKLKNAATLIQRHWRG |
| | | HNCRKNYGLMRLGFLRLQALHRSRKLHQQYRLARQRIIQFQARCRAYLVRKAFRHRLWAV |
| | | LTVQAYARGMIARRLHQRLRAEYLWRLEAEKMRLAEEEKLRKEMSAKKAKEEAERKHQER |
| | | LAQLAREDAERELKEKEAARRKKELLEQMERARHEPVNHSDMVDKMFGFLGTSGGLPGQE |
| | | GQAPSGFEDLERGRREMVEEDLDAALPLPDEDEEDLSEYKFAKFAATYFQGTTTHSYTRR |
| | | PLKQPLLYHDDEGDQLAALAVWITILRFMGDLPEPKYHTAMSDGSEKIPVMTKIYETLGK |
| | | KTYKRELQALQGEGEAQLPEGQKKSSVRHKLVHLTLKKKSKLTEEVTKRLHDGESTVQGN |
| | | SMLEDRPTSNLEKLHFIIGNGILRPALRDEIYCQISKQLTHNPSKSSYARGWILVSLCVG |
| | | CFAPSEKFVKYLRNFIHGGPPGYAPYCEERLRRTFVNGTRTQPPSWLELQATKSKKPIML |
| | | PVTFMDGTTKTLLTDSATTAKELCNALADKISLKDRFGFSLYIALFDKVSSLGSGSDHVM |
| | | DAISQCEQYAKEQGAQERNAPWRLFFRKEVFTPWHSPSEDNVATNLIYQQVVRGVKFGEY |
| | | RCEKEDDLAELASQQYFVDYGSEMILERLLNLVPTYIPDREITPLKTLEKWASRFYEAYK |
| | | FSGPSLPKNDVIVAVNWTGVYFVDEQEQVLLELSFPEIMAVSSSRECRVWLSLGCSDLGC |
| | | AAPHSGWAGLTPAGPCSPCWSCRGAKTTAPSFTLATIKGDEYTFTSSNAEDIRDLVVTFL |
| | | EGLRKRSKYVVALQDNPNPAGEESGFLSFAKGDLIILDHDTGEQVMNSGWANGINERTKQ |
| | | RGDFPTDSVYVMPTVTMPPREIVALVTMTPDQRQDVVRLLQLRTAEPEVRAKPYTLEEFS |
| | | YDYFRPPPKHTLSRVMVSKARGKDRLWSHTREPLKQALLKKLLGSEELSQEACLAFIAVL |
| | | KYMGDYPSKRTRSVNELTDQIFEGPLKAEPLKDEAYVQILKQLTDNHIRYSEERGWELLW |
| | | LCTGLFPPSNILLPHVQRFLQSRKHCPLAIDCLQRLQKALRNGSRKYPPHLVEVEAIQHK |
| | | TTQIFHKVYFPDDTDEAFEVESSTKAKDFCQNIATRLLLKSSEGFSLFVKIADKVLSVPE |
| | | NDFFFDFVRHLTDWIKKARPIKDGIVPSLTYQVFFMKKLWTTTVPGKDPMADSIFHYYQE |
| | | LPKYLRGYHKCTREEVLQLGALIYRVKFEEDKSYFPSIPKLLRELVPQDLIRQVSPDDWK |
| | | RSIVAYFNKHAGKSKEEAKLAFLKLIFKWPTFGSAFFEVKQTTEPNFPEILLIAINKYGV |
| | | SLIDPKTKDILTTHPFTKISNWSSGNTYFHITIGNLVRGSKLLCETSLGYKMDDLLTSYI |
| | | SQMLTAMSKQRGSRSGK |
| 9 | MYO7A | MDLRLGQEFDVPIGAVVKLCDSGQVQVVDDEDNEHWISPQNATHIKPMHPTSVHGVEDMI |
| | isoform | RLGDLNEAGILRNLLIRYRDHLIYTYTGSILVAVNPYQLLSIYSPEHIRQYTNKKIGEMP |
| | 8 | PHIFAIADNCYFNMKRNSRDQCCIISGESGAGKTESTKLILQFLAAISGQHSWIEQQVLE |
| | protein | ATPILEAFGNAKTIRNDNSSRFGKYIDIHFNKRGAIEGAKIEQYLLEKSRVCRQALDERN |
| | | YHVFYCMLEGMSEDQKKKLGLGQASDYNYLAMGNCITCEGRVDSQEYANIRSAMKVLMFT |
| | | DTENWEISKLLAAILHLGNLQYEARTFENLDACEVLFSPSLATAASLLEVNPPDLMSCLT |
| | | SRTLITRGETVSTPLSREQALDVRDAFVKGIYGRLFVWIVDKINAAIYKPPSQDVKNSRR |
| | | SIGLLDIFGFENFAVNSFEQLCINFANEHLQQFFVRHVFKLEQEEYDLESIDWLHIEFTD |
| | | NQDALDMIANKPMNIISLIDEESKFPKGTDTTMLHKLNSQHKLNANYIPPKNNHETQFGI |
| | | NHFAGIVYYETQGFLEKNRDTLHGDIIQLVHSSRNKFIKQIFQADVAMGAETRKRSPTLS |
| | | SQFKRSLELLMRTLGACQPFFVRCIKPNEFKKPMLFDRHLCVRQLRYSGMMETIRIRRAG |
| | | YPIRYSFVEFVERYRVLLPGVKPAYKQGDLRGTCQRMAEAVLGTHDDWQIGKTKIFLKDH |
| | | HDMLLEVERDKAITDRVILLQKVIRGFKDRSNFLKLKNAATLIQRHWRGHNCRKNYGLMR |
| | | LGFLRLQALHRSRKLHQQYRLARQRIIQFQARCRAYLVRKAFRHRLWAVLTVQAYARGMI |
| | | ARRLHQRLRAEYLWRLEAEKMRLAEEEKLRKEMSAKKAKEEAERKHQERLAQLAREDAER |
| | | ELKEKEAARRKKELLEQMERARHEPVNHSDMVDKMFGFLGTSGGLPGQEGQAPSGFEDLE |
| | | RGRREMVEEDLDAALPLPDEDEEDLSEYKFAKFAATYFQGTTTHSYTRRPLKQPLLYHDD |
| | | EGDQLAALAVWITILRFMGDLPEPKYHTAMSDGSEKIPVMTKIYETLGKKTYKRELQALQ |
| | | GEGEAQLPEGQKKSSVRHKLVHLTLKKKSKLTEEVTKRLHDGESTVQGNSMLEDRPTSNL |
| | | EKLHFIIGNGILRPALRDEIYCQISKQLTHNPSKSSYARGWILVSLCVGCFAPSEKFVKY |
| | | LRNFIHGGPPGYAPYCEERLRRTFVNGTRTQPPSWLELQATKSKKPIMLPVTFMDGTTKT |
| | | LLTDSATTAKELCNALADKISLKDRFGFSLYIALFDKVSSLGSGSDHVMDAISQCEQYAK |
| | | EQGAQERNAPWRLFFRKEVFTPWHSPSEDNVATNLIYQQVVRGVKFGEYRCEKEDDLAEL |
| | | ASQQYFVDYGSEMILERLLNLVPTYIPDREITPLKTLEKWAQLAIAAHKKGIYAQRRTDA |
| | | QKVKEDVVSYARFKWPLLFSRFYEAYKFSGPSLPKNDVIVAVNWTGVYFVDEQEQVLLEL |
| | | SFPEIMAVSSSRGAKTTAPSFTLATIKGDEYTFTSSNAEDIRDLVVTFLEGLRKRSKYVV |
| | | ALQDNPNPAGEESGFLSFAKGDLIILDHDTGEQVMNSGWANGINERTKQRGDFPTDSVYV |
| | | MPTVTMPPREIVALVTMTPDQRQDVVRLLQLRTAEPEVRAKPYTLEEFSYDYFRPPPKHT |
| | | LSRVMVSKARGKDRLWSHTREPLKQALLKKLLGSEELSQEACLAFIAVLKYMGDYPSKRT |
| | | RSVNELTDQIFEGPLKAEPLKDEAYVQILKQLTDNHIRYSEERGWELLWLCTGLFPPSNI |
| | | LLPHVQRFLQSRKHCPLAIDCLQRLQKALRNGSRKYPPHLVEVEAIQHKTTQIFHKVYFP |
| | | DDTDEAFEVESSTKAKDFCQNIATRLLLKSSEGFSLFVKIADKVLSVPENDFFFDFVRHL |
| | | TDWIKKARPIKDGIVPSLTYQVFFMKKLWTTTVPGKDPMADSIFHYYQELPKYLRGYHKC |
| | | TREEVLQLGALIYRVKFEEDKSYFPSIPKLLRELVPQDLIRQVSPDDWKRSIVAYFNKHA |
| | | GKSKEEAKLAFLKLIFKWPTFGSAFFEVKQTTEPNFPEILLIAINKYGVSLIDPKTKDIL |
| | | TTHPFTKISNWSSGNTYFHITIGNLVRGSKLLCETSLGYKMDDLLTSYISQMLTAMSKQR |
| | | GSRSGK |
| 10 | BEST1 | ATGACCATCACTTACACAAGCCAAGTGGCTAATGCCCGCTTAGGCTCCTTCTCCCGCCTG |
| | DNA | CTGCTGTGCTGGCGGGGCAGCATCTACAAGCTGCTATATGGCGAGTTCTTAATCTTCCTG |
| | | CTCTGCTACTACATCATCCGCTTTATTTATAGGCTGGCCCTCACGGAAGAACAACAGCTG |
| | | ATGTTTGAGAAACTGACTCTGTATTGCGACAGCTACATCCAGCTCATCCCCATTTCCTTC |
| | | GTGCTGGGCTTCTACGTGACGCTGGTCGTGACCCGCTGGTGGAACCAGTACGAGAACCTG |
| | | CCGTGGCCCGACCGCCTCATGAGCCTGGTGTCGGGCTTCGTCGAAGGCAAGGACGAGCAA |
| | | GGCCGGCTGCTGCGGCGCACGCTCATCCGCTACGCCAACCTGGGCAACGTGCTCATCCTG |
| | | CGCAGCGTCAGCACCGCAGTCTACAAGCGCTTCCCCAGCGCCCAGCACCTGGTGCAAGCA |
| | | GGCTTTATGACTCCGGCAGAACACAAGCAGTTGGAGAAACTGAGCCTACCACACAACATG |
| | | TTCTGGGTGCCCTGGGTGTGGTTTGCCAACCTGTCAATGAAGGCGTGGCTTGGAGGTCGA |
| | | ATCCGGGACCCTATCCTGCTCCAGAGCCTGCTGAACGAGATGAACACCTTGCGTACTCAG |
| | | TGTGGACACCTGTATGCCTACGACTGGATTAGTATCCCACTGGTGTATACACAGGTGGTG |
| | | ACTGTGGCGGTGTACAGCTTCTTCCTGACTTGTCTAGTTGGGCGGCAGTTTCTGAACCCA |
| | | GCCAAGGCCTACCCTGGCCATGAGCTGGACCTCGTTGTGCCCGTCTTCACGTTCCTGCAG |
| | | TTCTTCTTCTATGTTGGCTGGCTGAAGGTGGCAGAGCAGCTCATCAACCCCTTTGGAGAG |
| | | GATGATGATGATTTTGAGACCAACTGGATTGTCGACAGGAATTTGCAGGTGTCCCTGTTG |
| | | GCTGTGGATGAGATGCACCAGGACCTGCCTCGGATGGAGCCGGACATGTACTGGAATAAG |
| | | CCCGAGCCACAGCCCCCCTACACAGCTGCTTCCGCCCAGTTCCGTCGAGCCTCCTTTATG |
| | | GGCTCCACCTTCAACATCAGCCTGAACAAAGAGGAGATGGAGTTCCAGCCCAATCAGGAG |
| | | GACGAGGAGGATGCTCACGCTGGCATCATTGGCCGCTTCCTAGGCCTGCAGTCCCATGAT |
| | | CACCATCCTCCCAGGGCAAACTCAAGGACCAAACTACTGTGGCCCAAGAGGGAATCCCTT |
| | | CTCCACGAGGGCCTGCCCAAAAACCACAAGGCAGCCAAACAGAACGTTAGGGGCCAGGAA |
| | | GACAACAAGGCCTGGAAGCTTAAGGCTGTGGACGCCTTCAAGTCTGCCCCACTGTATCAG |
| | | AGGCCAGGCTACTACAGTGCCCCACAGACGCCCCTCAGCCCCACTCCCATGTTCTTCCCC |
| | | CTAGAACCATCAGCGCCGTCAAAGCTTCACAGTGTCACAGGCATAGACACCAAAGACAAA |
| | | AGCTTAAAGACTGTGAGTTCTGGGGCCAAGAAAAGTTTTGAATTGCTCTCAGAGAGCGAT |
| | | GGGGCCTTGATGGAGCACCCAGAAGTATCTCAAGTGAGGAGGAAAACTGTGGAGTTTAAC |
| | | CTGACGGATATGCCAGAGATCCCCGAAAATCACCTCAAAGAACCTTTGGAACAATCACCA |
| | | ACCAACATACACACTACACTCAAAGATCACATGGATCCTTATTGGGCCTTGGAAAACAGG |
| | | GATGAAGCACATTCCTAA |
| 11 | BEST1 | MTITYTSQVANARLGSFSRLLLCWRGSIYKLLYGEFLIFLLCYYIIRFIYRLALTEEQQL |
| | isoform | MFEKLTLYCDSYIQLIPISFVLGFYVTLVVTRWWNQYENLPWPDRLMSLVSGFVEGKDEQ |
| | 1 | GRLLRRTLIRYANLGNVLILRSVSTAVYKRFPSAQHLVQAGFMTPAEHKQLEKLSLPHNM |
| | protein | FWVPWVWFANLSMKAWLGGRIRDPILLQSLLNEMNTLRTQCGHLYAYDWISIPLVYTQVV |
| | | TVAVYSFFLTCLVGRQFLNPAKAYPGHELDLVVPVFTFLQFFFYVGWLKVAEQLINPFGE |
| | | DDDDFETNWIVDRNLQVSLLAVDEMHQDLPRMEPDMYWNKPEPQPPYTAASAQFRRASFM |
| | | GSTFNISLNKEEMEFQPNQEDEEDAHAGIIGRFLGLQSHDHHPPRANSRTKLLWPKRESL |
| | | LHEGLPKNHKAAKQNVRGQEDNKAWKLKAVDAFKSAPLYQRPGYYSAPQTPLSPTPMFFP |
| | | LEPSAPSKLHSVTGIDTKDKSLKTVSSGAKKSFELLSESDGALMEHPEVSQVRRKTVEFN |
| | | LTDMPEIPENHLKEPLEQSPTNIHTTLKDHMDPYWALENRDEAHS |
| 12 | BEST1 | MFEKLTLYCDSYIQLIPISFVLGFYVTLVVTRWWNQYENLPWPDRLMSLVSGFVEGKDEQ |
| | isoform | GRLLRRTLIRYANLGNVLILRSVSTAVYKRFPSAQHLVQAGFMTPAEHKQLEKLSLPHNM |
| | 3 | FWVPWVWFANLSMKAWLGGRIRDPILLQSLLNEMNTLRTQCGHLYAYDWISIPLVYTQVV |
| | protein | TVAVYSFFLTCLVGRQFLNPAKAYPGHELDLVVPVFTFLQFFFYVGWLKVAEQLINPFGE |
| | | DDDDFETNWIVDRNLQVSLLAVDEMHQDLPRMEPDMYWNKPEPQPPYTAASAQFRRASFM |
| | | GSTFNISLNKEEMEFQPNQEDEEDAHAGIIGRFLGLQSHDHHPPRANSRTKLLWPKRESL |
| | | LHEGLPKNHKAAKQNVRGQEDNKAWKLKAVDAFKSAPLYQRPGYYSAPQTPLSPTPMFFP |
| | | LEPSAPSKLHSVTGIDTKDKSLKTVSSGAKKSFELLSESDGALMEHPEVSQVRRKTVEFN |
| | | LTDMPEIPENHLKEPLEQSPTNIHTTLKDHMDPYWALENRSVLHLNQGHCIALCPTPASL |
| | | ALSLPFLHNFLGFHHCQSTLDLRPALAWGIYLATFTGILGKCSGPFLTSPWYHPEDFLGP |
| | | GEGR |
| 13 | BEST1 | MFEKLTLYCDSYIQLIPISFVLGFYVTLVVTRWWNQYENLPWPDRLMSLVSGFVEGKDEQ |
| | isoform | GRLLRRTLIRYANLGNVLILRSVSTAVYKRFPSAQHLVQAGFMTPAEHKQLEKLSLPHNM |
| | 4 | FWVPWVWFANLSMKAWLGGRIRDPILLQSLLNEMNTLRTQCGHLYAYDWISIPLVYTQVV |
| | protein | TVAVYSFFLTCLVGRQFLNPAKAYPGHELDLVVPVFTFLQFFFYVGWLKVSLLAVDEMHQ |
| | | DLPRMEPDMYWNKPEPQPPYTAASAQFRRASFMGSTFNISLNKEEMEFQPNQEDEEDAHA |
| | | GIIGRFLGLQSHDHHPPRANSRTKLLWPKRESLLHEGLPKNHKAAKQNVRGQEDNKAWKL |
| | | KAVDAFKSAPLYQRPGYYSAPQTPLSPTPMFFPLEPSAPSKLHSVTGIDTKDKSLKTVSS |
| | | GAKKSFELLSESDGALMEHPEVSQVRRKTVEFNLTDMPEIPENHLKEPLEQSPTNIHTTL |
| | | KDHMDPYWALENRDEAHS |
| 14 | CFH | ATGAGACTTCTAGCAAAGATTATTTGCCTTATGTTATGGGCTATTTGTGTAGCAGAAGAT |
| | DNA | TGCAATGAACTTCCTCCAAGAAGAAATACAGAAATTCTGACAGGTTCCTGGTCTGACCAA |
| | | ACATATCCAGAAGGCACCCAGGCTATCTATAAATGCCGCCCTGGATATAGATCTCTTGGA |
| | | AATGTAATAATGGTATGCAGGAAGGGAGAATGGGTTGCTCTTAATCCATTAAGGAAATGT |
| | | CAGAAAAGGCCCTGTGGACATCCTGGAGATACTCCTTTTGGTACTTTTACCCTTACAGGA |
| | | GGAAATGTGTTTGAATATGGTGTAAAAGCTGTGTATACATGTAATGAGGGGTATCAATTG |
| | | CTAGGTGAGATTAATTACCGTGAATGTGACACAGATGGATGGACCAATGATATTCCTATA |
| | | TGTGAAGTTGTGAAGTGTTTACCAGTGACAGCACCAGAGAATGGAAAAATTGTCAGTAGT |
| | | GCAATGGAACCAGATCGGGAATACCATTTTGGACAAGCAGTACGGTTTGTATGTAACTCA |
| | | GGCTACAAGATTGAAGGAGATGAAGAAATGCATTGTTCAGACGATGGTTTTTGGAGTAAA |
| | | GAGAAACCAAAGTGTGTGGAAATTTCATGCAAATCCCCAGATGTTATAAATGGATCTCCT |
| | | ATATCTCAGAAGATTATTTATAAGGAGAATGAACGATTTCAATATAAATGTAACATGGGT |
| | | TATGAATACAGTGAAAGAGGAGATGCTGTATGCACTGAATCTGGATGGCGTCCGTTGCCT |
| | | TCATGTGAAGAAAAATCATGTGATAATCCTTATATTCCAAATGGTGACTACTCACCTTTA |
| | | AGGATTAAACACAGAACTGGAGATGAAATCACGTACCAGTGTAGAAATGGTTTTTATCCT |
| | | GCAACCCGGGGAAATACAGCCAAATGCACAAGTACTGGCTGGATACCTGCTCCGAGATGT |
| | | ACCTTGAAACCTTGTGATTATCCAGACATTAAACATGGAGGTCTATATCATGAGAATATG |
| | | CGTAGACCATACTTTCCAGTAGCTGTAGGAAAATATTACTCCTATTACTGTGATGAACAT |
| | | TTTGAGACTCCGTCAGGAAGTTACTGGGATCACATTCATTGCACACAAGATGGATGGTCG |
| | | CCAGCAGTACCATGCCTCAGAAAATGTTATTTTCCTTATTTGGAAAATGGATATAATCAA |
| | | AATCATGGAAGAAAGTTTGTACAGGGTAAATCTATAGACGTTGCCTGCCATCCTGGCTAC |
| | | GCTCTTCCAAAAGCGCAGACCACAGTTACATGTATGGAGAATGGCTGGTCTCCTACTCCC |
| | | AGATGCATCCGTGTCAAAACATGTTCCAAATCAAGTATAGATATTGAGAATGGGTTTATT |
| | | TCTGAATCTCAGTATACATATGCCTTAAAAGAAAAAGCGAAATATCAATGCAAACTAGGA |
| | | TATGTAACAGCAGATGGTGAAACATCAGGATCAATTAGATGTGGGAAAGATGGATGGTCA |
| | | GCTCAACCCACGTGCATTAAATCTTGTGATATCCCAGTATTTATGAATGCCAGAACTAAA |
| | | AATGACTTCACATGGTTTAAGCTGAATGACACATTGGACTATGAATGCCATGATGGTTAT |
| | | GAAAGCAATACTGGAAGCACCACTGGTTCCATAGTGTGTGGTTACAATGGTTGGTCTGAT |
| | | TTACCCATATGTTATGAAAGAGAATGCGAACTTCCTAAAATAGATGTACACTTAGTTCCT |
| | | GATCGCAAGAAAGACCAGTATAAAGTTGGAGAGGTGTTGAAATTCTCCTGCAAACCAGGA |
| | | TTTACAATAGTTGGACCTAATTCCGTTCAGTGCTACCACTTTGGATTGTCTCCTGACCTC |
| | | CCAATATGTAAAGAGCAAGTACAATCATGTGGTCCACCTCCTGAACTCCTCAATGGGAAT |
| | | GTTAAGGAAAAAACGAAAGAAGAATATGGACACAGTGAAGTGGTGGAATATTATTGCAAT |
| | | CCTAGATTTCTAATGAAGGGACCTAATAAAATTCAATGTGTTGATGGAGAGTGGACAACT |
| | | TTACCAGTGTGTATTGTGGAGGAGAGTACCTGTGGAGATATACCTGAACTTGAACATGGC |
| | | TGGGCCCAGCTTTCTTCCCCTCCTTATTACTATGGAGATTCAGTGGAATTCAATTGCTCA |
| | | GAATCATTTACAATGATTGGACACAGATCAATTACGTGTATTCATGGAGTATGGACCCAA |
| | | CTTCCCCAGTGTGTGGCAATAGATAAACTTAAGAAGTGCAAATCATCAAATTTAATTATA |
| | | CTTGAGGAACATTTAAAAAACAAGAAGGAATTCGATCATAATTCTAACATAAGGTACAGA |
| | | TGTAGAGGAAAAGAAGGATGGATACACACAGTCTGCATAAATGGAAGATGGGATCCAGAA |
| | | GTGAACTGCTCAATGGCACAAATACAATTATGCCCACCTCCACCTCAGATTCCCAATTCT |
| | | CACAATATGACAACCACACTGAATTATCGGGATGGAGAAAAAGTATCTGTTCTTTGCCAA |
| | | GAAAATTATCTAATTCAGGAAGGAGAAGAAATTACATGCAAAGATGGAAGATGGCAGTCA |
| | | ATACCACTCTGTGTTGAAAAAATTCCATGTTCACAACCACCTCAGATAGAACACGGAACC |
| | | ATTAATTCATCCAGGTCTTCACAAGAAAGTTATGCACATGGGACTAAATTGAGTTATACT |
| | | TGTGAGGGTGGTTTCAGGATATCTGAAGAAAATGAAACAACATGCTACATGGGAAAATGG |
| | | AGTTCTCCACCTCAGTGTGAAGGCCTTCCTTGTAAATCTCCACCTGAGATTTCTCATGGT |
| | | GTTGTAGCTCACATGTCAGACAGTTATCAGTATGGAGAAGAAGTTACGTACAAATGTTTT |
| | | GAAGGTTTTGGAATTGATGGGCCTGCAATTGCAAAATGCTTAGGAGAAAAATGGTCTCAC |
| | | CCTCCATCATGCATAAAAACAGATTGTCTCAGTTTACCTAGCTTTGAAAATGCCATACCC |
| | | ATGGGAGAGAAGAAGGATGTGTATAAGGCGGGTGAGCAAGTGACTTACACTTGTGCAACA |
| | | TATTACAAAATGGATGGAGCCAGTAATGTAACATGCATTAATAGCAGATGGACAGGAAGG |
| | | CCAACATGCAGAGACACCTCCTGTGTGAATCCGCCCACAGTACAAAATGCTTATATAGTG |
| | | TCGAGACAGATGAGTAAATATCCATCTGGTGAGAGAGTACGTTATCAATGTAGGAGCCCT |
| | | TATGAAATGTTTGGGGATGAAGAAGTGATGTGTTTAAATGGAAACTGGACGGAACCACCT |
| | | CAATGCAAAGATTCTACAGGAAAATGTGGGCCCCCTCCACCTATTGACAATGGGGACATT |
| | | ACTTCATTCCCGTTGTCAGTATATGCTCCAGCTTCATCAGTTGAGTACCAATGCCAGAAC |
| | | TTGTATCAACTTGAGGGTAACAAGCGAATAACATGTAGAAATGGACAATGGTCAGAACCA |
| | | CCAAAATGCTTACATCCGTGTGTAATATCCCGAGAAATTATGGAAAATTATAACATAGCA |
| | | TTAAGGTGGACAGCCAAACAGAAGCTTTATTCGAGAACAGGTGAATCAGTTGAATTTGTG |
| | | TGTAAACGGGGATATCGTCTTTCATCACGTTCTCACACATTGCGAACAACATGTTGGGAT |
| | | GGGAAACTGGAGTATCCAACTTGTGCAAAAAGATAG |
| 15 | CFH | MRLLAKIICLMLWAICVAEDCNELPPRRNTEILTGSWSDQTYPEGTQAIYKCRPGYRSLG |
| | protein | NVIMVCRKGEWVALNPLRKCQKRPCGHPGDTPFGTFTLTGGNVFEYGVKAVYTCNEGYQL |
| | | LGEINYRECDTDGWTNDIPICEVVKCLPVTAPENGKIVSSAMEPDREYHFGQAVRFVCNS |
| | | GYKIEGDEEMHCSDDGFWSKEKPKCVEISCKSPDVINGSPISQKIIYKENERFQYKCNMG |
| | | YEYSERGDAVCTESGWRPLPSCEEKSCDNPYIPNGDYSPLRIKHRTGDEITYQCRNGFYP |
| | | ATRGNTAKCTSTGWIPAPRCTLKPCDYPDIKHGGLYHENMRRPYFPVAVGKYYSYYCDEH |
| | | FETPSGSYWDHIHCTQDGWSPAVPCLRKCYFPYLENGYNQNYGRKFVQGKSIDVACHPGY |
| | | ALPKAQTTVTCMENGWSPTPRCIRVKTCSKSSIDIENGFISESQYTYALKEKAKYQCKLG |
| | | YVTADGETSGSITCGKDGWSAQPTCIKSCDIPVFMNARTKNDFTWFKLNDTLDYECHDGY |
| | | ESNTGSTTGSIVCGYNGWSDLPICYERECELPKIDVHLVPDRKKDQYKVGEVLKFSCKPG |
| | | FTIVGPNSVQCYHFGLSPDLPICKEQVQSCGPPPELLNGNVKEKTKEEYGHSEVVEYYCN |
| | | PRFLMKGPNKIQCVDGEWTTLPVCIVEESTCGDIPELEHGWAQLSSPPYYYGDSVEFNCS |
| | | ESFTMIGHRSITCIHGVNTQLPQCVAIDKIKKCKSSNLIILEEHLKNKKEFDHNSNIRYR |
| | | CRGKEGWIHTVCINGRWDPEVNCSMAQIQLCPPPPQIPNSHNMTTTLNYRDGEKVSVICQ |
| | | ENYLIQEGEEITCKDGRWQSIPLCVEKIPCSQPPQIEHGTINSSRSSQESYAHGTKLSYT |
| | | CEGGFRISEENETTCYMGKWSSPPQCEGLPCKSPPEISHGVVAHMSDSYQYGEEVTYKCF |
| | | EGFGIDGPAIAKCLGEKWSHPPSCIKTDCLSLPSFENAIPMGEKKDVYKAGEQVTYTCAT |
| | | YYKMDGASNVTCINSRWTGRPTCRDTSCVNPPTVQNAYIVSRQMSKYPSGERVRYQCRSP |
| | | YEMFGDEEVMCLNGNWTEPPQCKDSTGKCGPPPPIDNGDITSFPLSVYAPASSVEYQCQN |
| | | LYQLEGNKRITCRNGQWSEPPKCLHPCVISREIMENYNIALRWTAKQKLYSRTGESVEFV |
| | | CKRGYRLSSRSHTLRTTCWDGKLEYPTCAKR |
| 16 | ABCA4 | AGTCCCCAGTCTTTGCTTAGGCCCCTACGTACACAAACTGAACCTAGTGACCCAGCATGGCCTCTAATTT |
| | DNA | CTCAACACTTCTGTACTTCTGTAATGATTAACCCATGCTTCTCACAGATCCATGCCCCAAATTTCTGTGA |
| | | ATAGGCCCTGACTGGCCCAGCTAAGATCATGTGACTGCACATGACCAGTCCACTTTGGCATTAACAAGCC |
| | | TACTGCAGACTCTTCCCTTGGTGTTGGAGTCACTCCTAGAAAAGAGCAAATCTTTGTGAGCCAGGCAGTC |
| | | AACCTGCTGGCAGCTTCCACTCAGCCTTGGAGTTTTTTCTATGTGTAACTTTCATAAACTGAGCCTTATT |
| | | TATTTATTTTTTGCACTATCATCTCATGAAATATTATTGCGTAAGCTGAGGAAACATGTTATTCATGATG |
| | | ACTGGAGTTTCAAGTTTTAATTGTACAATGATTTAGTTTTGAGTTTGGTAGAAATAAAATCAAATTTAAA |
| | | AATCAGATATTTTTCATCTTACATTATGATGTCCCAAAACTGCCTTTATGCTTGTGACATAGATTCATAA |
| | | TGTCTTCTCATTCCACCTGTAATCACTGTTTGAAATAAACATATGTCTAATGATATATTTGGGGACATTC |
| | | TATTTTCTTCAGCTTGTTGCAAGTGAATTGATGGTGATCTTTTGGTATTGGTTTCATTATCAAATTTATC |
| | | TCCACTCCAAAATTACAGTAATTTCAAAGTAATTTAGTCTATATATTTTTCCATAGCTTTTCTTCCAAAT |
| | | AGAAACTGTAAAAAGTTATAAATTACTTCTCTCCACTACTGAATTTTTGTTTGCAGAATAACTGATGTAA |
| | | GTAGCAGAATGCCTCTTCCTAGTTCAACCCTCAGGAATAGAAGTGAGAAGATCTTTAAAACTTCACCATT |
| | | TTCCTTGACTTGTTTATAATTCTGAATGTAAATGTGAATTGATATGGTCTATCGCTTAACACCACAACTC |
| | | TTAATCTATGTGCAGGGTCGTAGCTCAAAACTACTGCCAGGACCACATCAATTTCATATTCACCCTGATC |
| | | AATGTGTATTAATGGTGATAACTATGAGAATGAAATGTACAGTTATCAGTATCATTTTTGACTCACTAGG |
| | | TATATCCTCAGAAATATATGAAAAAACTAAACACAGCTTTTAGTTTGACATAATTTTTAAACAACTGGAG |
| | | TTACCTTGGGAGAAAAATCCTACCAAATATCTATAATATTGAAAGAGTAAAAAAGAGTTAAATGTCCTTA |
| | | ACATCATTAATCATTAGGGACATGCAAATCAAAACCACAGTGAAATACCATCTCACACCCTTTAGGATGG |
| | | TGGTGATAAGAAGAAAAACAGAGCATAACAAGTGTTGGCCAGGATGTGGAAAAGCTGGAACCATTGTGCA |
| | | CTGCTGATTGGAAAGTACAATGGTGCAGCTGCTAAGGGAAATAGTATGGTAGTTCTTCAAAAAATAAACA |
| | | GTTATACCATTTGATTCAGCAGTTTCACTCCTAGGTATATACCCCAAAGAATTGAAAGCAGAATCTCAAA |
| | | TATTTGTACACCTATGTTCATAGCAGCATTACTCACAATAGCCAAAAGGTGGAAACAACCCGAATGACCC |
| | | TGGATGGACGAATGGATAAACAAAATGAGGTCTATACTGACAATAGGATATTAATTGACCTTAAAAAGGA |
| | | AAGAAATTCTGGCCGGGCACGGTGGCTCACACCTGTAATACCAGCACTTTGGGAGGCCAAGGCAGGCAGA |
| | | TCACCTGAGGTTGGGAGTTTGAGACCAGCCTGACCAACATGGAGAAACCTCATATCTACTTAAAATACAA |
| | | AAAAAAAAATTAGCCAAGCATGGTGGCGCCTGCCTGTAATCCCAGGTACTCAGTAGGCTGAGGCAGGAGA |
| | | ATCGCTTGAACAGGAAGCAGAGGTTGCAATGAGCTGAGATTGCACCATCACACTCCAGCCTGGGCAACAA |
| | | GAGTGAAACTGCATCTCAAAAAAAAAAAAACAAAAAAAACAAAAAAAAGAAATTCTGACACATCTGCTAT |
| | | GGTCTAAATTATGTGTTCCTCTAAAATTCATAAATTGAAATCCTAACCCCCAAGGTGATGGTATTAGGAG |
| | | GTGAGGCTTTGTGGAGGTGATTAGGTCATGAGGGTACAACCCTCGTGAATGGGACTAGTGCCCTCATAAA |
| | | AAGAAGCCCAAGAGAGACCCCTTTTCCCTTCCACTGGATGAGGTCACACCAAGAAGTTACCATCTACATG |
| | | TCAGGAAATAGGCCCTCACCAGACACCAAATCTATTGGCACCTTGATCTTGGACTTCCCAGCCTGCAAAG |
| | | CTGTGAGAAATAAGTTCCTGTTGTTTATAAACCACCCAGTTTATGGTATTTTGTTATAGCAGCTCAAACA |
| | | GATTAAGATGGCTTGCTACAACATAGATGAACTTTAAAGATTATGCTTTTCTATGATTCCACTTAGATGG |
| | | GGTACCAAGAGTAGTCAAGTTCATTGAGACAGAAAATAGAGTGGTTGGCAGGGGCTAGGGGGAGGGAACT |
| | | CTGGGGAGTTAGTGTTTAATGGATACAGAGTTTCAGTTTTGCAAAATGAAAAAGTTCTGAAGATGGATGG |
| | | TGCTGATGGCTGCACAATATGAATGTATCAACACTACTCAACTGTACACTTAAAATAAGGTTCAAATGAT |
| | | ACATTTTATTTCATGTGTGTGTCAATCTCAACAAACAGATTTGTTCAGGCAAGGAAACTGGTTAGATGCG |
| | | AATAATACTATTAGAGCATCATCAATTGAATATTAACAAAGTGCTCATAGTTTAACTTTCTAGCTCAAGG |
| | | AAGAATGGACCATTTTGAAACTATGACAGAACATTACTTATATAGCTGATGTCTTTGGGAATTGGAAGGA |
| | | GGCATATTCCTTCACCAGCTGTGGCTCCCCTTCAGCAACCTCATATACTCTCCAAGCTTCTCTTTCCTGG |
| | | GTCACCTGTTTAATCACTCCCGGGACTTAATCTTCCACCTATATGTTGACCACTCACAAATCTATGTCTC |
| | | CATCTCACAAGCTTATTCTTGACTCCAGACCCAAGTATTCAACTGCCTGCTGAATACGTGTGGTCAGATG |
| | | TCATAGAACTTCAGCTTCAGTATATCAAATGCAAACCCCTGTTCCCCCCAACTGCCTCCTACTCCCCACT |
| | | GGCCTTCCTCTGGCATTCCCTCCTCAGTTATGAGCACCACCGTCTCACTAGCCAGCCAGTCAAGCCCCAA |
| | | ACTCCATCTAGCTGACTTCTGCCTCTTCCTCACCACCCTCTTCCAGTAACTCATCAGGCACTGCTGTGTC |
| | | TCATTCCTTCCTATCCCTCCAGTCCCTCCCCTTCTCTCCATCATGGCTGTCACTGCATGGTTCAGGCTCT |
| | | CTGGCTCCCCCCAAACCACCCCCACATTGCTGCCGAGGTGAACTGACTACTCTTGGCAGCCACTGGATTA |
| | | AAATCTTTCATCATCTTCAGCATGATAAAACCCATATCCTTTAGCATGTAACAAGGTCTTAATGATTCTG |
| | | CCAGAGCTTGCTTGGGGGTAGCCTGCACTTGTGGGCCACTCCAGTCACTTCACAGGTGCTCAGTAAATCT |
| | | CAGTTGAATCAGTCATCATCATCATCATCATCATCATCATCATCATCATCATCAATTTTTCAGTCTGGTT |
| | | CCTGTCTCCTTTTCCAGCATCCTCCATTCATAGCCTCATAGCCTTCACTCCAGCCATGTTTCACTTGTGG |
| | | TTTTCCTGGGCAAGATAAGCTATTCCTCCCTGTCTTTGCAGAGTTTAAATGACTCACTTGTTCAAGTACC |
| | | CACCGTTGCCATGTGGGACCGTGAGCAAAGTACTTAATCTCACTAAGCTTCACGTTCCTCATCTGTAAAA |
| | | CAGCAAATATGGACCTCACAAAATTGTAGTGAGGCTAAAATGAAATAACATATGCAAAAGCAGTTTATAA |
| | | ATAATAAACTTACTATAAAATATTATTTTGTAATTCTGCAAGCTTGTCTTAAATGCCATCACCTCCAAGG |
| | | AGCCTTTTTGCCATCATAAGCAGAAACTATCTCTCTCTTCTTGGAAGCTCCACCATGCACAGCCTATGGG |
| | | CCCTCATCACACTCCTTGAGTTATTCGAGTTCAAGTCCCGTGTTTACAACCAGACCGCAAACTCTATGAA |
| | | GTCAGCATCCATTCCTCTCTGTGGTTCTCCCTCCGCCCCATCCAGGTCTCAAGGGTCTAGAGTCTTTCAA |
| | | AGAGAACACATTCTGAGATTTGAGGAGGCAGAGACAAAAAGTTCCACTGCGAAGTGCCAGGGAGGCTTCT |
| | | GTTTGGGGTGTCCCTTGGGATCACAGATCCCCCACCTGGTGATGAGTCAACCCAGCACCACCCCATTGCA |
| | | GGGCTGGAATGACAGTAATGGGCCCACCTGCTGCCTCTCCTCATACCCGCACCCCAGTCAGACATTGCAA |
| | | GTCAGTCACGGCTCTGTCCTGCTGGGCCTGGAGTGTTCCAGTGCCTTTTCCATCACAGCACCAAGCAGCC |
| | | ACTACTAGTCGATCAATTTCAGCACAAGAGATAAACATCATTACCCTCTGCTAAGCTCAGAGATAACCCA |
| | | ACTAGCTGACCATAATGACTTCAGTCATTACGGAGCAAGATAAAAGACTAAAAGAGGGAGGGATCACTTC |
| | | AGATCTGCCGAGTGAGTCGATTGGACTTAAAGGGCCAGTCAAACCCTGACTGCCGGCTCATGGCAGGCTC |
| | | TTGCCGAGGACAAATGCCCAGCCTATATTTATGCAAAGAGATTTTGTTCCAAACTTAAGGTCAAAGATAC |
| | | CTAAAGACATCCCCCTCAGGAACCCCTCTCATGGAGGAGAGTGCCTGAGGGTCTTGGTTTCCCATTGCAT |
| | | CCCCCACCTCAATTTCCCTGGTGCCCAGCCACTTGTGTCTTTAGGGTTCTCTTTCTCTCCATAAAAGGGA |
| | | GCCAACACAGTGTCGGCCTCCTCTCCCCAACTAAGGGCTTATGTGTAATTAAAAGGGATTATGCTTTGAA |
| | | GGGGAAAAGTAGCCTTTAATCACCAGGAGAAGGACACAGCGTCCGGAGCCAGAGGCGCTCTTAACGGCGT |
| | | TTATGTCCTTTGCTGTCTGAGGGGCCTCAGCTCTGACCAATCTGGTCTTCGTGTGGTCATTAGCATGGGC |
| | | TTCGTGAGACAGATACAGCTTTTGCTCTGGAAGAACTGGACCCTGCGGAAAAGGCAAAAGGTAACAGTTA |
| | | CTGTCTGTGGTTTAAAAATGAGGTGTGGAGCAAATAAACAGGTTGGAAGTGTGGGGTGGGGTGGTGGGGT |
| | | AGGGTGGTGGGGCAGGGTGGGGGGTTGTGAGCAGTCAGTGGGCTTGTCGCCGATTAGCACTGAAGCAGTG |
| | | TTTAGCTGGACGGCCTTTCTGTGGGCCCCTCTGACAGTGCCCTTCCCAGGAAGATGTGTTTCTCTGTCCT |
| | | CAGCCACATGAAAATCTTTTGCCTACCGTGCCTGTCAATCCATTGCCTGCCCGCCCCTCCCCCACCCCCC |
| | | GTTTTACACCTGCCTGTCCAGTCTACCGCTCTCTAGGGCATCCACGCTGAGCAGTGGGAAGAACTTTAAG |
| | | CCCTGAAGAGCAGGCCAAAGGCAAGCAAGAACCCCCTCGAACAGCTTCCCAGCTTAGTGAGGCCTTATTT |
| | | CATTGATTCTCTGAGGCACATTGTTTTTTCACATGTTAGCATTTCTGAAATTGGGATGCAGCTCACGATC |
| | | AAGTCACAGTTTAACTGGACACATTATTTTTCTTTCTTAGTGGTGCAGAAAAGTAACAGTGTGTCTTACA |
| | | ATTGACTGCGTCCTAGATTCTGTGAGATGCAATACGTTATTAACCATCACGCACATTTCCTGAACTCTTT |
| | | CAATGAGCAGACACCAGCCTGGGTTAGACTGGAGCCCTAAAAGCACGACACAGATTCCACCCTGGACTGG |
| | | CTTCTGTTCTGCCTGGGAAAACCCAAAGTACGTTTGGAGACCAAGAGCAACATAAAGTAGCATAGGTGGA |
| | | ATAGTCCATGAGAAGTGCGAGCAAAAGGTGCCGGAGATCAGAGAACACCAAGACTGTACTTGTAAATGAC |
| | | AACTGGCTTTGTGCAATTTTTTCTGGGAAAGGATAAGGAGTGACTATAGAACTGTAAAGAAAGAATGCAC |
| | | TTTGCTACAGCCTTGCAGAGTTGTGCAAATGCCGATGACTAAAGGAGCTGAAAGAGGAAGGAGGGGATAA |
| | | GGGATGGGGGCTGGGTAGGGGTGAGATTAGGACCCTGGGAGCTGCAAGCCACTGGAGAGATCAGGAGGAA |
| | | AGGGAGGGAGACCTGCTTTAGGCGAGAAGAGAACAGTATTTGTTCCAAATCTCGGTTCAGAATAAGTTCA |
| | | TGTAGGTGATGGGGCCAACTGGAACAGGTGAAGGCCTATGAATGAGTGTCTCAGTTAGGGTCTCCTTAGA |
| | | GTTTAATATGAAAAGGTGTTAGCTAAGTACAGAGCTGGTACCTGAGAGAGTAAAAGGAAACTCTAAGGTA |
| | | TCATGGAGGTAGCAATTGCAGGACACAGCTCCCACCCCTAGGGCTGAGAGAACCAAGGGAAGAGACAGGA |
| | | ATTATTAAGACTTGGAGCATAGATGAGAGGTCTGTGGAGCTGACATTAGGACTTGGGAGGAAGGCGTGCA |
| | | TGGAGGCTGCTGCTGGATCTCTGAACCTGACCTCGGGTCTGGACCCCTGAGGAGAAAGCCCTGGCAGGTT |
| | | GGTGCATGTGGGGCCGAGGGACAATAGCTTAACAACCAGCATAAAAGAGAGCAGCATGGGACACGCTTCA |
| | | ACCATGCGCATGGATGGCTCCAAAACCTGTGTGTGGCTGGCCCAGGACGCAGGGAGGCTGCAGGGGGAAG |
| | | AGACAAGTTAAACCTGACTTGTCTGGGAAGCACCATTGTCCTCAGGTCACTTTCCTCTGTCAAGCCTGGT |
| | | GCTGAAGTTATCTGTTGTCTCCAGGGGCCAAGTATTAAGAGTAATCAGAAACTCAGTCCTTTCTTCTAGG |
| | | AGCTTCCCTTCTTGCATGAAAATCCTGATAAAACTGGAAAAAAAAACCTCATGATTAAATTTTTTCATGT |
| | | ATTCATTCTTTCCTTCTATCAAAAAATAATCTCCAGGCACCGTGCTAGGTTCATTGGTATACAATGGCAA |
| | | CAAGACCTCCCAGCCCCTGCCTATGTGAGGCATCTGTGGACTGCGGAGGAAAATCCAATATGCCATTGTT |
| | | CTCTCTTTCCCATAAGAAATTACAATTCTCAGTTCATTTTATTCTCACTGTGCTCTTTGTGACCCTCAAA |
| | | GGGGGTCACATGATAACAGGACTGTAGCTGCTGGCCTAAAATGAGCCCATTCCTGTGGCGCTCATGTCGC |
| | | TGTGACAGAGAATAACCCTGTTTTCAGAATGCTCTGGTGCCCTCCCTCTCAATCTGGCCTTTCGCTGGCA |
| | | TGGGTGGGCGACTCCTGCTCAGGGACTCTGCCTTCTCCACAGTGTGCTCCCAGGGAGATGGAGCCACTCG |
| | | GGCTGAGGGCCTTGGCCAGGGCACCTCCCAGGGCTGGGCCTGGTCTGGGCTGGCGTTCACTGGATGCCAT |
| | | CCTGATGGCCTGGAAATTGAGATTTCTGTCTGGCACGCCTCCCGATGGCTCCCCACCTGCTACCACATTC |
| | | CAGGAGCTTCCAGGATGTCTGGGTAAGACAGAGGCACCCCCAACAGATTCAGTAGCTCTGAGAGGGATCT |
| | | GTGGCTCCTTCCTAAGCTTGCGGTTCTTCTGGAAACTTCTGCCTCTAGAAGATGGTCCCTCTAAGAAAAG |
| | | TACAACCACCCAGCCCATAATTCAGCTCCCAGGTTTTCCCTCAAACCTCCATGTCTCCTGTAAGCAGAGC |
| | | AAGAGTAAAATCAGATACCAAATTTCCTCATTCCTCAGCTCCCAATCCCTAAGGGCATAAGATGAAAATC |
| | | TTCAGATCTCTGCTTTCCTCCCTCTTTTTTTCTTCCTCTGTTAACATTTGTCAAGTGTTACTAAGTGTCT |
| | | GGCACTGTACTAAGTGCATCACCTCCCTGAACTCTCCGAACAGTTCCACGAGAGAGGCCTCTCTGTGATC |
| | | CCCCCGGTACTGATGAGGTCACTGAGGCTCCAGAGAAGGATTAGTAACTGGTGGGGTTGGACCTGGGATT |
| | | CACACCCATGCTGCGTGACCCAGGACAGGCAGGCATGGCCGTTACACCACACTGACCCCCGTGGATCGAG |
| | | ATCTATCCAATAGTCTGGTCACTGATATCACTAAGATAGAGTGGCCATATAATTTATCATCCAATCAGGG |
| | | CAGTTTTGCAAGTGAAAGGGAGCACTATTAATAATTGCACTGGGACAATAAATGTAAACCAACACTGGAC |
| | | CTGGAAAACTGGGACGTGTGTTTGCCCTATACCAAGGTAAGCTAGACACAGCCACTGCCTTCATGGAGTT |
| | | CAGAACCAGGCAGGGGCGGCTCCCACGTATAATTACTGTGCAGCACAACGTGGAGACCGTGGAGTAGAAG |
| | | GAAACACGGATGGGAGGTGAGGAGGAGGTCTGTGAGCTCAGAGGAGGCACCGGGGCTGGAGAGGGTGAGA |
| | | GAAGACTTCCCAAGGAGTTCATCCTGATAACGTGCATTCCCAATGACGAGCGCTCTCTCCACTGCACAAG |
| | | ACAAGTATACATCTGCCCGTGTTGGCTGTGGACCTGGCGCTGTGTCAGGGAGGGTTTATGAAGATCACTA |
| | | GGTGGGTCTCTTGGTGTCATCCCTTCATCCCAGCTTCTGGGTTAGGATGGATATCTGTGGGGGGGCCTGA |
| | | GGACTCATGAAAGTGGGGCGCTAATCATGTTTTGGACACCACACCCTGGAGCACCTGGGACAGCTGTGGC |
| | | CTTTGTCCTGGGTTCAGCATCAAGCCGAGGATGTGGCAAGTAAAGAGAGGCTGGGCACCAACTCCAGTGT |
| | | ACCCAGGCTCCGGGTCATGTTTGTCCAGGCTAAGAATTCTGTCCTGGTTCTCAGTGCAGAAGGAAGAATC |
| | | ATGGGGCTCATTTTAGGCCTTGGCTGCCTTCTGTTAAATTGAAAACAGAGCAGGAAGGAAGAAAATTTAA |
| | | CAGGCTCAGTTCTAAAACAACAAGCACAACTGTGCCCTTGCCAGAAACCCCTCCTCCCCATGTTGATTGA |
| | | ATGGTAAAGAGAGGAGGGGAGGTGAGAGGGAGAGAGAGAGAGAGGAAGAGAGAGAGAAAGGAAAGAAAGG |
| | | AAAGAAGAAGAAAGAAAGAAAAGGAAAGAAAGAAAGAAAGAAAGAAAGAAAGAAAGAAAGAAAGAAAGAA |
| | | AGAAAGAAAGAGAAAGAAAGAAAGGAGGGAGGGAGGGAAGGGGAAAAGAAAAGAAAAGAAAAAGAAAAAA |
| | | AGAAGGAAATACCAGTTTGGGAAAAAAGAATTTTCCACCAGCCCTTCTGAGCCTTGGCTGGGCTTAATTA |
| | | AAGTTACAGACATGTGTAAAGGGCAGGGTAGGGGGAGTCTGAGCTGCTGAGAAAACATGTTTTTAATTAT |
| | | ACTGTGGAATTTCTCCCTGGGGTATGCCTGTACGCAGTTAAGCGTCAAGGACAGGGATGCCGCTCTGGGG |
| | | AGGGGAAGCTGAGCATGATTTTGGAAGCCGGCAGAAGAGGCTATTGTGAAAACCAGACCTGTCAGGCTAG |
| | | GAAAAGAATGGCTGGTGGTCTTTGACCAGGGAGTGACGCGTGAAATGCAGCAACCGCCCCCGCCCCCCGC |
| | | CAAAAACAAACACACTCTCACAGAGTTAGAACAACAGTGACCTCTCAACAAATATTTTTCAAAGATTACC |
| | | AACCAACCATTACCTAGAGCAGCGGTTCTCAACCTTGGCTGCACGGTGGAACTACCTGAGACGTGTTAAA |
| | | AAGAAGAACCCTGATGTCCCATGCCCCAAGATTCTGATGTAGTTGATCTGGGGTATGATCTGAGACCCCG |
| | | GCATGTTTTCAGCCTGCAGCCACATGAGAAGTGCTGACCTAATCAACAGGGGTGATGATTTGAGGGGCGG |
| | | GGACTATAGGCAAAAAAAAACAGCCTAATTCAAGGATGAGAAGAGGGCACAGGTGAGGTGGGAACAGTCC |
| | | TAGGGCCAGACAAAGAAGGAAGGGAGAAAGGAGGTGCTGATCCCTCCCCTACTCCTGAGAGGAGGCCTTT |
| | | AAGTCACCGTGCCTTGTGGAGACCAGATTCTTCAAAAATACAAGAATGAGTGAGTGAGGGAGTGGGTGGA |
| | | TGCCAGGAGAGTGCGTGACAAGCCTTGCAAGGGAGGATGACAATGCACTAGCTTGGTTTGGAAATTTTAC |
| | | CCCTGGAACAGGCAGGCCAAGCTGGCTGGTCCCCTCCCTGATACACAGCCCTCCCTCTTTATATATGGAG |
| | | CAGGGGACGGTGTGTGGCTGGTTTCTTAGCAAGCACCATGGTTCCAAGTTGGCAACTGGGGAGTTCTGAA |
| | | TCCAAAAAGGAGGGAGATGAACGTAAGTGGAGGGCAGGCCTACAAGGTTGCAGATAAGCTTAATTCTGTC |
| | | TCCTTACTCTTCTGCCTTTGCAACAACCCTGTGATCTTGCGACAACCCTGTAAGGCAATAACAAATGGCT |
| | | CATGTTTATTGAGTGTTACCTCATGCCATATTGTGCTTTCGTGTTTAACACAATTGTCTCATTTCACCCT |
| | | CACGACTGCTCTGGGAGGTAGGTCCTGGTATCACATCCATTTCACAGATGAGACCATTTGGCACGGAAGA |
| | | GTTGAGTGGGCTGCCCAAGGTCACATAGCTAAGATGGAACAGGCTGGATAGGAACCCCAGTAACTTGACC |
| | | TCAGAGTAACCTTCTCTTAACCCTGAGTGTACACTGTAGGAAAAATGAGCAGTCCCATTTCAGAGAGGAC |
| | | AAAACTGAGACTCAGAGGTTAAGCAAGCCCCAAAGTGGTTGTTAACCCAGATCTTCCCACTAACTCCCAA |
| | | ATCAGCATCAGTGTTTAACGTACCAGACCTCTCCCAGATAGATGTTGCCGCATGGAAGACAGCCGATCTA |
| | | CGTGATAGAAAGCCAATATTGCAAGCAGTCGTCTAAAGGAGTCAAATGTGTTGGATTTGAACTGGATGTC |
| | | TCATTTCTTTGGTGAAGACACTGGAAACAACTTCCAGGTTTCATCAATTGCTCCTATCACTCAACGTTGC |
| | | TATCTTACTGAACTTGTTCCCCAGCCTTACCCACTGATGGAATGATCCAGAATGGAAGACAAGACACCAA |
| | | TGTACATGACCCTGGGGGAGGCTGTTTCTTAAATCTACAGACTGTTGGTGACCTGAGCCCCATGTCACCA |
| | | AAGGCTTTCCTGGAGAAGCCTCCTAGACCAGTCTTGACAAAGGCTCACTCATTCCGTGGATATTTATTGG |
| | | GCACCTATTATGAGTTCTGCCCCATGTGGGGTGCTGGAATCACAGTAGTGACAACGACAGATGAGGTTCC |
| | | TGTCCTCAGGAAGCTTACTGCCCTTGAGGGCTTCACTTACTTGGAGGAGTGATGAACCTGAAGTGCGGTG |
| | | TGTGTTAAGAAGCGGAAGTCCAGGGCCAGGCGCGGTGGCTCACGCCTGTAATCCCAGCACTTTGGGAGGC |
| | | TGAGGCAGGCGGATCACCAGGTCAGGAGATCGAGACCATCCTGGCTAACATGGTGAAACCCCGTCTCTAC |
| | | TAAAAATACAAAAAAATTAGCCGGGCATGGTGGTGGGCACCTGCAGTCCCAGCTACTCAGGAGGCTGAGG |
| | | CAGGAGAGTGGCGTGAACCTGGGAGGCAGAGCTTGCAGTGAGCCAAGATCGTGCCACTGCACTCCAGCCT |
| | | GGGCAACAGAGTGAGACTCCGTCTCAAAAAGAAAAAAAAAAGTGCCTCACGGAGAGTCTATTCTTTTCTT |
| | | CCCATATTGTGTGTGTGTGTGCGCGCTTCCTCCAACACATCCTCCCTATATATATTTTGAGTAAAACATC |
| | | TTGTAAAAAGTTACAGCTACATAATCACCACCTGTCCCTAAATAGTTTTTGCTTTTTCTTTCTTCAATGC |
| | | ACGATCATTTTCCCCCATCAATTTATTTTTTAGTTTCTTATAATCTTGTTGCCAGTAGGCTGTTTTTTAA |
| | | AAAGCAGAACATGGTTTGTTCTTACTAGCAGGAAAGGAGCATTTATTGAGCCTCTGCTATGGTGTCTTTT |
| | | ATTTTGCTGAGAGCCTATTTACATTTCTTTGAGAGGAAAACAACAAAGGGTTACATGAAAGACCATGTGA |
| | | ATAGCCCCTAGCTGATCTATTAAACTTGCTATTCCCCGGCCAGCTGCTTCAGATCTCCTTCAGATCTTAT |
| | | GTGTTTCCTTCCTAAGGTCCCTGGAGTAAGGGTTGCATAGACCTATTCTACTCTCCAACTCACATGTCCC |
| | | TCTCCCTCTTCCTCTCCATAATTCCACATCTCCAACCCCCACCCCTATGTGCAATGCCACAGGGTGTGGA |
| | | CTGCCACAGCCACTGGATCTGCTTTTGGAATCAAGAGTCCTTAAGCTCCAAATGGAACCGAAATTTAAAT |
| | | ACCAACTTTCAACCATATGTTAACATCAGCAGCCTCTTCCAATGTAAAAACCCATGGCAGTGTGCCCTGC |
| | | TTTGTTTCTTTAAGCAATAGAAACTTGAAGGAAGCATGTTGGTAGGCCAGATTTTTGTTGGCTTTGCAAT |
| | | GGATCACAGTCATTTATTCACTCATTCATTCACTGATTCATTAAATGACCACATTTGCAAGGGCAAGGTA |
| | | ATGGGGAGGGCCAGAAAGGACACTGGCCCCAGAAACAGGAGGCTGGATTTTGGTTCTGATGCTGCCACTG |
| | | CTGATGTGACACTGCACAGGTCACCTGCCTCCTCTGAGCCTCTTTCCTTAACTGCAGAGTGAGTGGCTAC |
| | | AGAGAAATCTTTACTACCTGTTAGATCAGCATTACCTGGGAGCTTGTTAGAAATGCAAGCTCTGGTGGGG |
| | | CCATACTGAACCCAAATCTGCATTCATGTGCATAGTGACAGCTAAAATGCACTGAAGCAGATGATCTTGA |
| | | TGATCCTTTATGAAAGTCTCATGCTAATGCAGTTTTCTAAAATAGAGGCAGAGTGGAACCCAGATGGACA |
| | | CAAAATCTGGTTGATATAATAAAACAAGGTAGAGGGTGTATGGTGGGGAGGGGGTAAAGGAAGGAAACTG |
| | | TTTAGGTAAAGATACCACAACCAAAGTCCTACTGCACACATGGGATCTGAGGAGGGCTGTGTCTGCTCTG |
| | | GTTACGTTTTCTATAATCTCTTAGCACCACTGAACTTTCTCTCTTTTTGTTTTGTTTTTCCAGATTCGCT |
| | | TTGTGGTGGAACTCGTGTGGCCTTTATCTTTATTTCTGGTCTTGATCTGGTTAAGGAATGCCAACCCGCT |
| | | CTACAGCCATCATGAATGTAAGCATAGCAGGGTAGCTTGGGCAAGCCCTGAAGAGACTTTGGTCTGGGCC |
| | | TTTTGTCTAGAAAGATCTTGGGGTGGGAGTGTGGGGATCAGATCTGCTTATCATCATTTCATGTCTATGA |
| | | TGCATGTAACAGATTTATCAATGTTACACAAATTATAATTTTTAAAAAGTCTTTAGAGACAGGGTCTCAC |
| | | TCTGTTGCCGAGGCTGGAGTACAGTGTTAGGACCATGGCACACTGCAGCTTCTATCTCTTGGGCTCAAGT |
| | | GATCCTCCTGCCTGGGCTTCCAAAGTGCTGGAATTATAGGCATGAGCCACTGCTCCCAGCTAATTTTTTT |
| | | GTTTTTTGTGGAGACAGAGTCACTACATTGCCCGGGCTGGTCTTGAACTCCTGGCCTCAAGTGATCCTCC |
| | | CACCTCAGCGTTCTAAAGCACTGGGATTACAAGCATGAGCCACCTTGTCCAGCCCAAATTTTCATGTTTT |
| | | AATCCTACACATTCTAAGCAAATACTTGTGTGTAGTTACTAAGGGACTGTGCACTTATTTTTGTTTGCTT |
| | | TGTTGTTGCTAGTTTTTATTTTTTTATACCTAAACTCTCTCGTTTTAAAGAGAACAGATTTGTAGATGAG |
| | | TTCTCGAAAATATTTCAGGAATCAATATAGAGAATATGTTATACATGGTGCCAGAGAAAAATGAGGACAA |
| | | GAGATGCTATACAATCGTACTGAAGAAAAATTTTATTTCTTGGACCCCTGAGGTGTCTGCAGACCTGAAA |
| | | GGAACCTAGTGAGAGCCTCTTTTACACTCTGCCCCTGTGGGAAAGCCTTCACCTGGTTTCCGGCCCTCTA |
| | | TGTGGTGAATGTGGAAGCCTCAAGCGTTATGCAAATCTGCCCAGTCCTCTATTCTTGATCTTCACCTTCT |
| | | CGTTCATGAGTTTCAGGCCCCAGTTCTGAATCAGCCTCCTGTCCATCAGACTCTTCTTTACCTCTCCCCG |
| | | AGGAGCCCATAACCTGCAGCCCTACTGCATGCTTGGGGTAGGTGCTCAGTTCACCGTGGTTGAAGGAATA |
| | | GACGAGCGTCTGCTCAAGCAGCAGCAGCAACTGCGTGGAGTCTTCTTGAACTAACACTCCTATGCCCCTC |
| | | TCGGCACAAAATGACGTGTCCCCCCTTGCTTCCCCTTCACATTTCCACCCATGCCTATTACAACATCCGT |
| | | CTGTCTCCCCACTACACCGGGAGCTTGAGAGAAGAGGCCATGTCTCTAGCACCCAGCACAGGGACTGGCA |
| | | CACATGAGATGCTCCTGCTTCTTAAATGCTGAGAATGAAGGAGGACATCAGAGGGGCCCGGGCCCCTTCC |
| | | CAAAAAGGCCAACTCCTAGGTCTGCATCCTGCTTGGTCTCCATGACTAATCCCGTCTTGTCCTCATTTTC |
| | | TGTTTTAAAGGCCATTTCCCCAACAAGGCGATGCCCTCAGCAGGAATGCTGCCGTGGCTCCAGGGGATCT |
| | | TCTGCAATGTGAACAATCCCTGTTTTCAAAGCCCCACCCCAGGAGAATCTCCTGGAATTGTGTCAAACTA |
| | | TAACAACTCCATGTAAGTGTTGAGATCCCTACCATGCAGGGGAGGAAGTTGCACACCCCTTCACGTGCTG |
| | | AAATGCACACGTGCGTGCACGGAGCATGGAGCACTGAGTGTTCTTGTGGCTTTGCTGAGCCCCTAACCTC |
| | | TTAGGAGCAGAGCAGGTTTCCTCTCTGGAACATTCTGTTAACTGTCAGGGCACTTGGGGAGAAATCTCCA |
| | | AGCTAAGGCCACGTGCACAAAATTTCTTGGTCCTTATATCCCCAGAATGTGACCTGGAGTCTGATGGCAG |
| | | CCCGCTGCAGAGATGTGTCCACTGCCTTCTGGTCATTGACCTGCTTGGGTGGAGTGAATCATTGTAGGAG |
| | | AAAAACTCAGTTCCCTCACCCTGATCAACCTGGACAGATCTCTCTTCCTTTAAAAGCTTTCTTGGACATC |
| | | TAAGGGCTAGGAAAAATGTCAGGGAGCATTGGGAAGGTAAATGAAGTCAGGTTTACAAAGTCAAGTTTAC |
| | | TTCTTGGGAGAAAAATACAATTTCCAAATCCTCTGTTATAATTGCCATCGGCCCCCTGGAGTGGTGAGAT |
| | | CTCGGAATATGGCTCGGGTGCAGTGGCTCTTCACTGTGGGCCTGCAGGCTATTCTGAAAAGCTGATGAAA |
| | | ACCAATGACCCCTCTTCCAAGAAAAATGGCCACATACCAAACATTACACTGTACATCTGATTTCAGGGAA |
| | | TTGTAGATGCCAGGTTAGTAGCCTCAGGTCTAGGGTCAAAATTCAAGTCGAATCCCACAGGAAGAGGGTC |
| | | TGCCTTCGGAATTCCCTTTCAGAGCATTGGGAGAACATCATGGGAGCATATTCTAGAGACAGAGGCTTAG |
| | | GGTGTGGACAGGGCCATCCCTCACCCACTGTGCTGACCTTAAGCAGCACCTTGTGCAGCCCATACCTGAA |
| | | GGCCACCAGCAAAGGCCTGTTGGGGAGCAGGCTTTACCCGACCTGTATAAACACCAGGCTAGGTGAAAAC |
| | | TGAGATACCTGGTTACTTTAGTTTTTTCCTTGGGGGAGCTCAGTATGATTCTTCCAGGAGAAGCCTGCTT |
| | | TTAGACTAAAAAGAAAAAAAGTTTGATAGGTCAACCTAATGATTGGAGGTGGCCTTCCCCACTGTGAACA |
| | | AACTATGGCTGCATGTGCCCTACAATGGCAGAGTTGAGTAGTTGTGATAGAGACTGTATGATCTGTAAGC |
| | | CTGTAATTTTTATGTTTGCTGACCCCTGGATTACCAGATGATAGAAGAGGAAACATCTGTCTTCCTAGCA |
| | | AAGTCAAGGAAGTGGCATTTAGCAGGACTCATATTGCTGCAAGCACTGCCTTGCAGTTTTAGTTTACAAC |
| | | TGCACTTTCAGCTTAAGAAACACCTGCCCATCCAGAGAGATCGTGTGGGGTCACATGGTGGGATCAGGGA |
| | | GGCCTGAAGACAGCTCAGTGGAGGCTGCATGGAGCTTTGGTGGGAACGGCCCTGGCAGTGTCTATAGATG |
| | | TTATTGCGGAAAACTGAGGGGTGGGAGTTGGAGAAGGGGGCTCCAGACTCTAGCTGTACTTGGCATTTGA |
| | | ACCCGGAAAGTTGGGTTTCATGTTTTGCACTCACATTATGAGTGAAATATTGGCTTATTCAAGGTTCTTT |
| | | TGCTTGCAAGGCACGGAAACCCATTCAAGCAATCTTAAACCCCAGAAGGAAATCTATGATTTGGATACTA |
| | | GACATTCTCACAGAGCCAAGGGCAGCAAGGCGGGGCTCAGGAGAGGCAGGCCAAGACCTGGAGAGCTGTC |
| | | AGGAGCTGCTTCCTCAACTCTCTTCCATCTGGGCCTGCCAGCCCTGGCCTCTGTATCTACTCCATTCACC |
| | | TCTCTCCATGGACCAGTCTCCCCTGCTCCTCAATGCCTGGGCTGCCATTGTTCATGCAATTCACAATACC |
| | | TCGGCCTGGGCAATCAGAAGCTCATCTCTGAACACCATCCAAATTCCTGGGAACAAATCGGGTTGACCCA |
| | | GCTTTATTCTCCCTGTCCCATCAGCCTTGGCAGAGGCGTGCATGTGCATGCGTGCCAATGTGTGTGTGCA |
| | | GGGAGGTCCTTGTGGATGAAGCATGGCTGTCAGAGCCTACCTGCGTGAATGGGTGGAAGGGCAGGTCTCA |
| | | GAGAATTGGGTAAAAACTGGATAAACCCTCCAGTGATATCCACCAATGTCACCCTGTTTAAGGCTTCTCT |
| | | GGGCAAGAGACACACAGAGCATGGGACCGAGAGGCGAGCAGACCCTGCCAAAACTGGGAGACTGAATAGA |
| | | TCGCTCACCATCCTTGTCAGTTAGCCTATATGTACAAGGAAGTAAAATTATCTCTTTCTCCTGCCTTGGC |
| | | AGTATTGTAAGGATACTCAATGTAGTAGCTAGGCCAGACACATAGTATCTTTAAATATAGCATGAGATGG |
| | | CCAAGCACGGTGGCTCATGCCTGTAATCCCAGCACTTTGGGAGGCTGAGGCGGGTGGATCACGAGGTCAG |
| | | GAGATCGAGACCATCCTGGCTAACACGATGAAGCCCCGTCTCTACTAAAAATATAAAAAATTAGCTGGGT |
| | | GTGGTGGCGGGCGCCTGTAGTCCCAGCTACTCGGGAGGCTGAGGCAGGAGAATAGCGTGAACCCGGGGGG |
| | | CAGAGCTTGCAGTGAGCCGAGATCACGCCACTGCACTCCAGCCTGGGTGACAGAGCGAGATAAAAAAAAA |
| | | AAATAGCATGAGATATTATTACTGTTATAAAAATAACAGCTATTTCCTTATTAATGAGGCTTTGTCCTTA |
| | | CAGCTTGGCAAGGGTATATCGAGATTTTCAAGAACTCCTCATGAATGCACCAGAGAGCCAGCACCTTGGC |
| | | CGTATTTGGACAGAGCTACACATCTTGTCCCAATTCATGGACACCCTCCGGACTCACCCGGAGAGAATTG |
| | | CAGGTAAGCATGACTGCAGTGCTCTCAAGCATCATTTCCCTCACCTATGGAGAGACTGAAGATATAGGAA |
| | | AGAACAGGGAGAGTTGGTGAAAAATATACTAGCGGAGGCAGGAAGGGATGGGGTCTGGAGGCGGCTTGAA |
| | | CATCACCTTGGTGAAGATGCCTCTTCCTCCACAGAAGCCTGGAAGGTAGGAAGTTGGGAAGGAAGGCAGG |
| | | AAAGGTCTCATCCACGTTAAGTCTAGAGACAGAAAGAATGCTAAGAGAGATGGCACTATGGGAAGTATGA |
| | | GGCTAGGTCAAGGGCTAGAAGCAGGGGAGACGAGTTTACAGAGTTTCGTAAAGATATAGAGCAACTCTCA |
| | | CAGAGTTCTAGAGCGAGAGCTAACCAGGAACATGAAGCAGCAAGGCCAACTATCATTAAGGAGCCAGGGA |
| | | GGTCAGAGATCATGTATTATCATGACATAAATATGCATAATTGTACTATTTCTCCCAGTAATATTTAGCA |
| | | CCCAGGCCCCGAGGCAGAGCAAGTGGAGAGTGGGTGATGCAGGGCTGGGGGTGTGTATGGAGGCACCACA |
| | | GAAGGTCAACAGGCAGCGGGCTGAAGGCAGGGACTGGACTACATGCATCAAGTCCAGGCTGCACGAGGAA |
| | | GGATGAGAAGGCAGATGAGCACGGAAATGGACTGGGGGAAATGAAGAGGCAAGGGAATAGAAGTCTCAGT |
| | | GGGTGCCATGACCCTGTTTAAGTGATTGAGAAAATGAACAAGATGAAAAGGTTAATGGCTGTGGTCAGAA |
| | | AGTGAAATATGTGAATTCAGGATTTCGAAGGTAGGGTGGGTGATGACTGGCCCCCAGATGCGGCCATGGT |
| | | GAAGTGGGGCAAAGGTGCAGGTGCATGGTGAGGGGAAGGAGGAAATGGGAGGTGATGATGTTGGCCCCAC |
| | | ACGGACACCACGGTTGTGCAGGAAGATGGCAGGAGCTGGGCACCAGGGTGGGAGCCACCTGGAGTCAGGA |
| | | AGAGTGAAGAGAAAGGATGAAGAGGCTCCCTCTCCTGTGTCTCTCCTCCCCAGGAGAAGAACAAGAAACA |
| | | ATCCGAAAGTAATAACACCAATGTGCCTTTACAAAGTGTGAGTGGGTGTTGTGTGCTGTCACGTGTGTAG |
| | | TAGGCTCCTCTGTGGATGGCTAGAGGGACTGGACATGGCCACTGGATCCCACTTGCAAGAGCAGAGGAAA |
| | | AGAGTGGTCGTGAGGAAGTAAAGCCCCCCAAAATCCAGGGGTTGCTGCAGCTTTGGGTGTGGAGCGTGCC |
| | | CTCTGAGGAAAGGCTGCTCTGGGGGAGATTGCCCAGGAAACGGGGCTCAGAGGCCACGAAAGCAGCTGTT |
| | | AGGGGCTTCTGGGAGATGTGTGCTCCTAGGATTAGGGAGTTGACTCTAAGGATGACCTTAGAGGTTAACA |
| | | GGGATGAGAAAGGGGTCACCAAGGGGTCTACCAGGGGAATGGGAGAGGCTGTATTGATAGAACAGCTTCT |
| | | GCTGCAGGTTCCAAACAAGAAATGTGGGAGAATGGTTGAAATCAGCCCCGGGGGCACCTTCCCGTGCATG |
| | | CGTGCAGCTCCTTCAACATTCAGTCGACCTTCAGTGCCTCCTGTGAGCCAGGCACTGGGCTAGTCTCTGG |
| | | GGGTGGAGAGATGAGTCAGGCAAATGCCAGCCCTCAGAGGGCTCACAGGGCAGAAGGTGAGAGATGAGTG |
| | | AGCAGAAAATGACCACAGCGCGTGTGGGGCCCAGTGGAGGGAAGGAGGGGATTCAGGAGCACAGGAGAGT |
| | | CAACAGGGGAAACTTCTCCGAGGAGAATCTGATCCTCCTCCCATCTGGCCACCTTCTGAAGCCCTCTCTC |
| | | CCCATCCAAGTGAGAAAGGACAGGCGTATGACCAGATTGGTGTATGAAGATGCTGAATTACGTTCTCATT |
| | | GTTTCAAACTAGTAAACCATAGATTTTATGTAGTAACTTCTACAAACTGCATTACAAACACTCCATTCTT |
| | | TGTTGCCCTGGGTAGAAGTTTATTTTAGTGAGCCCAAGTTTGAGGAACCTTATATGGTATGAGTACAATT |
| | | ACCATTTTAATAGTAAGAAATCCCCCTTCCCCTGTGTACCAACCAGAAGGTGTTTTTTTCCTAATTTAAA |
| | | CAAACAGATGCAGACGTGGGCTGTCCAGCTCCTGGCGGGATGACATACCTCATGCATCCAGTGGGTTTGA |
| | | TGATGAGGCAGACATTTCACTTAAGTGCCTGATCATCAGATTGAGTCCTGCTGGGAGGAAGTGTGAAGGA |
| | | AGTAATTTCAAACCACAGTTTCTCTGTGGCTTTTACAATGTGGATATGAGAACCAAAATCACTACTTCTT |
| | | AACCCCAGAGCAGGACTGATTTTGAATTGGTATGCAGGCGGTTCCTTCTGCAGGCTTCGGGCTGTGAGAA |
| | | GTCCCTAACAGAGCAAATCTGGGGACAAGGGCTCAGGAAAGGTTGGCCACGGCCCCCTAGGAATGGGGGC |
| | | TCTGCAAGATCCCTGGCCTTAGAGGCTGTGAGAGGGAACAGGGGTCCATCCCCAAGTAAGGGACACGGTC |
| | | TTTGAGGAAATCCCAGGCCAGGGCCTGAAGGGCACTGTCAGGAACACAGGCTGTTTCAGTCTGTTGAGAT |
| | | TCACCGGGGCGCTGCTCACTGTGAGCACGGACTCCTCAGGCCAATGTGGCAGAAGAGCCCACCTTTGAAA |
| | | GCGAGCGGGTGGGGGTGGCGGGGCTGGTGCTGGTGCGTGCTTCTGCACAGCCACCTGGGAAGGTATGCCG |
| | | CTGGTTGACCCAGGCAGAGGTTTTCTTTCATGGCAAACCTGCAGTACTGCATTCTCAGCAGGGAGGATTA |
| | | ATGGTAAAAGACCAGGCATGGAGCCCCCTTCCCTCTCCCTCGAAGCAAGCTCTGTGGTCTCTCAATCATC |
| | | TTTAAAACACCTTCTTCCCGGGAGCCTCCTACATTCTCCTGGCTTCCCTCCCACCCCCACCCTCAGCTCC |
| | | TGGGGCCTCAGCAGCCCCACCCCCAAGCCTCTAATCTTCCCAGGGAAGGGAACAAGAAGAACCACATTTT |
| | | AAACGAAATTTATTTTTCTTTCCTCAGGCTCCCAGTTCACATTTCTCCCTCAGGAGTCTAGGGAAGCTTC |
| | | TGTCTGGTATCGGCCTCCTCTTCACCTGGGCCCCCGCCCTCCTCAGGTGTACCAGAAGCCAGCACACTCC |
| | | CCCTTCCCCCCCAGAGCCACAGCAGCCCTGTCTCCTGGGTGGTCTTGTGTGCCAAGCCTGGGCAACATCA |
| | | CTCCCAGCTTTTCTTGTTTTGCCCCTTCTCCCCAGCAAGATATTTGTATGTAAGGTCAGGTGAGTGAGTT |
| | | AAAGAATAACGAAGAGATAAACAGTCAAATGGAGTCCTGACTGTCAGGTCAAGACAACAGTTATTTACTG |
| | | AATGCCTCATGTCATTCAACAGACATTTATTGAGACTCTGATTGGATGTCAGTCTTTAATGCTGGGTGTC |
| | | AGAGAGAGGTGACTTCAAGGGCTTGCATCTGTGCACCCAGCATTGCTAGGTACAATGAGGAGTATAATAA |
| | | AAGCAGGAGCCATAGCCCCCAACTCTCAAGAGATCTCCCATGTGTGTATGTCTGCATATGCGTGCGTGTG |
| | | CATGTGTGCGCATGTGTGCATGTGTGTGTGCATGTGTGTGCATGCGTGTGTGTGTGCGTGTGTTGGGGAT |
| | | GGTGTTGGTGGAGTGAGAGTGTACAAGGCTGTGTATGAAGGGGTAATTGGGAAAAGAACAATGGAGCTGG |
| | | CACCCAGGGACAGGAGGAAAAGCAGGAGGGCTGGGTTTGGAAGACAGCCGGATTTATGTTTTTGAAGAGG |
| | | GAAGACTAGAATATAAGGGAGCAGCCCTTCTCAGAGCCCTCCTCCTCCCTTCGGGCCCTGTGTCCAGCTT |
| | | TCCCCAAAGTCCTTGGATCTTTCCTATGCAAAGGGGAGTGACAGTGGGCACCACTCTCAGGGAACCCATT |
| | | ACTGTGAGAGAAGCCACTGTGCCACTGTGTGGTCGAACTTCAAGACCGGCTTCCCCTGCCCCAGCTGCAT |
| | | GGACAGGCCTGTGGGGTTGGCGCAAGACCCTTCCAGAGGAAACTAGCTGCAACATAAATCCGGATATGGT |
| | | GCTGTTCAGGGAAAGGCACAACCTGGGGATGAGAAGGGTGGCTGTCCAGCACACAGGGGCAGGCCTCTTG |
| | | GCCACTGGGGGAGGGGAGAATTTGGAGAGGAAGAGGATGGGATGCCGTGGAATTGGGACCAGGAAAGAAT |
| | | GGGGACATGTGATGGTTAAAGCTAGTTAGAGAAGAACTGGGAGATAAACAGTCACCCATGCCCCTGAAGC |
| | | ACTCGGGGTGAAGAGATTGGCATTTTCACGCACCCCAGTGCTTTCCCTTTGTGTTGAAGTCCCTTCGTAG |
| | | ACATCCAGGCCCATAAGGCTCTTCTCTGGCCAGAGCCTCATGAACTATAGCACTAGCAGGGTTGAGGCCA |
| | | AGCATTGGCCCTGGAAGCCAGCCGAGGAGGAGGGTGCTTGTGTGAATCTCCCAGGAGGGGTAAGAATTAT |
| | | ATTAATTCGATCATAATAAGCATTTATTGAGTGCTGTTTTGAGGCCTGGGAGCTAAGCACTTCACATTCC |
| | | TTACCCCGCATCAACAATCCTATGAGGTAGATGTGGAAAATGCAGACACGGGGACAGGCTCAATCACTTG |
| | | CCCCAAGGTCACCTTAACTGTTAGGTGTTCTTTATGCCTCCTTATAAAGAAACCCTGCTTCCCACAGGTG |
| | | TTGAGAGGAGCTGGAGGGAGCTTGACTAGGGCTCATCAGGCAAGCCCCGGCATGTGCCTGGCTCTCCTCT |
| | | TTCTACCTGGAGCTTTTCCTGCCCTTAATGGCCCCAACTCATTTCTCTTAGTCCATGTCAGTGCCCTGAG |
| | | CATCTCAGCCCAAGCTGAGATGATAGAAACACCCAGAGGGGTCCTCTACCCTGTGACAGCTGCGGTGTGG |
| | | GAAGAGCACGTGTCTCCTCCAATCCTAGACCAGAGTTTCTCAGCCTCAGCATCACTGACACTTGGGGCTA |
| | | GATAATCCTTTGTGTGGGGGAGGGAGGAGTGTCTTGGGCCTTGCAGGATGTTTAGCAGCATCTCTGGCCT |
| | | CTACCCACCAGCACCTCCCCAGTTGTGACACCCAGAAATGTCTTTAGATCTTGCCAAATATTTCCAGGAG |
| | | GATGAAATTCCCCTGTTTCAGTTCCCCAGCCCCACCTCAATGAGAAGCACTGTCCTAGACCAACCCCACA |
| | | AAGCATCTGACACCCCCATCCAGCCCTGGCTAACTTTTTCCACCTTCTTACTAAATTGGGCCCAGCTGCT |
| | | TCAGCAGTCAATGTGTTGGGGGCAGCCCACTGGCAAGAGCCTCACCTCTAGGGGCTCCCAGAGACCCCAA |
| | | GAACAGAACCTTCCTCTGAGAGTTGAGTTACAAGTGTTTCCAATCGACTCTGGCTGTTTTCCTTTTTTTG |
| | | ACCCATTTCCCCTTCAACACCCTGTTCTTTCTCTTATTCATATGTAGGAAGAGGAATACGAATAAGGGAT |
| | | ATCTTGAAAGATGAAGAAACACTGACACTATTTCTCATTAAAAACATCGGCCTGTCTGACTCAGTGGTCT |
| | | ACCTTCTGATCAACTCTCAAGTCCGTCCAGAGCAGGTAGGGGGATGTCACTGGCCAGTGGTCCCTGGAGG |
| | | GGAGGGAAGCACCCAGCCTGAGAAAGGCAAGAAATATATTGGCTTTTTTCTTCTTTCTTCCTTGTGTTCA |
| | | CATTCAGAATCCATCACTTAATGCCTTGTATTTAGAAAAAAACCGGGGGATCACTTGAGATCGTGATCAT |
| | | TTTCAACATAGGATTCGAAGCTGTACACATCCTGGTGACCTTAAAACATCTCAGGTTTTTATAACTGGAA |
| | | GGAACCTTAGAGATCATGGGGCACAACCTTCTCTTTATAGATGAGGAAACAGAAATCTATTCATTTATTA |
| | | CTCAAATATTTAGGGACAGTTGTAGGTACTAGAACACAGTGTGAACCAGACAGGCAAAACCCCAGGCCAG |
| | | GGAGCTTCCATTCCAGTGGGGCCACAGGCGATGCTCAGGTAAGCAGAGACTCCGCTGTGTGACTTCTGGC |
| | | TGTGATGGGTGCTGCAAGGAAAATCCGGTAGAGTCGAGGGTTAGAGAGGGACGGAGGGGCAGGTTTAAGG |
| | | GGGATGCTCAGGAAGGCCTTCCTGAGGAGGTGGTATTTGAGCAGAGTTGTCTGTCAGCCACACAGTAAGT |
| | | GAGAGGGGAGTTCCGGGCTTGGAAGCTGCCAGCACAGTGCTGGCAAGTGCTGGGGTGGCGTCCCGAGGCT |
| | | ACAGAACCTGAGATGCTGCAGAAGAGCCCACTTCTGCTTTCCTGGACCACTTCCTTCTCAGCACCAGGCA |
| | | AACTCCTTCTTCTATCCCCTGGCACATTTCTGACCTGTGTATACGCCCCCAATTTATCTAACCCCTTTAA |
| | | ATAATCTCCTCTATTTATGCAGAGCATTCTTACCACTAACTCACGACTTGCACATCCCTTAGCTCCCTTA |
| | | CTCCTCACAACAATCCTGAGATGGGTCAGAGAAGGAGGCTTGCGCGTCTGGTGATGGGGTGATTTGTGCA |
| | | CAGTTACAGGGCTAGAAATTGTCAGAGCCAGATGGAATCCAGGTCCTCTCAATCCTAATCCAGTGTTTCT |
| | | TACTTCAGTCCTGTGGCTCTCAAAGCCCAGAGACCAGCAGCATCAGCGATGCCTGGGAGCTTGTTAGGAA |
| | | TGCAAATTATCAGGGCCCACTCCAGGTGAACTGGGTCCAAAGCCCTGGGATAAGGCCTAGCAATCTGTGC |
| | | TTCACAAGCCCTCCAGGTGATTCCGCAGGCTCAGGTGTGAGAGCTGCAGCTGTCCTCTGGGCCTTCTGGG |
| | | CTCCCCGCCCAGCTTCTTCAGTGTGATGAACACAGCGAGAATGCTAGATCTGCAGCAGCTGATATCCCAG |
| | | ACACCCTCCCGACTCCCTCCTGGCTGGGTCTGATCCTCCTCCAGACTCCAGGAGAGAACGAGACATAAAC |
| | | AGAACTTCAGAGCCTGTGTTAACCCTGAGATCAAGGTCTGCACAGGGTGCTGTCTGAGTCCAGAGGAGTG |
| | | AGGGACCCCACCCCACCTGGTCAGCACCAGCTCCTGGAAGCAGGTTCTCACACTGGTTCCCTGCACAATG |
| | | AAGGAGCTCATACCTGCTTTTCTGGCTTCTCAGACCCTGAGGTTTTCACCGAAACTAGACAAGGGGAACC |
| | | TAGGGTCAGCCTGGAGGCAGGGTGAGCTTGGCGCCTGCAGTGCCCAGGCCCTGGGTGGTGCGGCTCCGGC |
| | | CAGGCCCTGTTTAGCTTCCTCTCCCACCCCCACAGAGGGGGTGCTGTCGGCACCGATTGCTCATTTTCCC |
| | | CTTTGCTTTCTCTTCAGCTCGTAAAACTCAAGTCCTGACAATGCCTTGATGACTTCCAGTTGGTAATAAA |
| | | AGGGAGATGAAGATAAGGACAGGAATTTCGGGGAAATTTCTTTCCAGTTCCTTACTAATGTGACATTTAG |
| | | ATCTCTAGTACTGTGCTTCTGGCATCAGTGCCAAGGCCTTTCATGTTGGAGAATGGAGGCCGGGGTCACC |
| | | AGGTTGTGCCTTTATTTCATGTTGCTGGCTCTGATGAGCTGATGCTCTGCTGATTAGCAAACGCTGAGCC |
| | | ATCTGCGCTTCGCAGAGGCACGTTCCAGCCAACCCGGCCCTCCCTGCCCACTTCCCAGGATGCTTTGCCT |
| | | TGTGGGCTCACCTGTCTTCTAGCTCCTGATCTGTATCTCCACCTCCATCCAGTTCCGGGGCTCCTTATCA |
| | | GCACTGTTCCCAGAACTGTCCATCACGATGGCAACGTTCTCTCTGGGCGCTGTCCAACATGGGAGCTCGC |
| | | CTCTGTGTTGTCACTCATGCTCATTGAACATGGATTTGTGTCCTTTACCATCAGGACTGGATACCCCTCC |
| | | TGGTCCTTTCTGCCTGGGGTCTTAGCACAGCTCAGAAGGAACCTCACCATTCCCTCTCTCCATCTAGGGA |
| | | ATTAGAAGATGACAGGGGCACAGTTCTCTGGCTCACCCCCAGCCCAGTAAACTCCTGGACATGCTTCAAG |
| | | GCCCAGCTCAGATGTTGCCTCCTCAGTGAAATAATTTATAAACCCACCCTTCTTTGTCCTGCCTTCTCCC |
| | | TCTTCCCTACTCACTGGAGAGTTAACAGGTGATGGTTAAGCTCTGGGTTCAAATCTCACAAGGCCACACA |
| | | CTTAGCTATGTGACTTCAGGCAAGTTAATTAACCACTCTGTGCCTCTCGTTTCCTCATTTGTAAAATGGA |
| | | AATAGTAAAAGTGCCTACCAGCATGGCAGTTGAAGTTAAAAGAAATAATATATGTGAACACTTGGAAGGG |
| | | CGCCTGACACATAGTAAACTCTCAGTAAATACTAGCTGCTTTTAGTGGCTATTCTTAACACACCCTCTTC |
| | | AGTGCTCTGGTTTCACTATGTTTTATGGGTCCCTGAGATCGAAAGTGTCCACACCGACTCATGGTCAGCT |
| | | GTAACCTGTGCCTCGTGTGGGGACCAGGCTGCCATGTGTAGTCTGGACAGTGTAGGAGGTGGCAGAGCTC |
| | | AGGCCTGTTCTGCCCTCCAGCCCAGAGAGCCACGTCGTTAGATGTCATGGGAGACTGTGGTGCCCCGGGA |
| | | ATCTCACGAATTTGCCCACGGTACTCAGTGTCTGTCCAATGCTATGGGAGTCCAGGACTCTAGGAGCCAG |
| | | TTAAGGTGCTGGGTGGCCACAGGTCCCTGGCCAAGGTCCAGGCCTCTCCCCTGCCACCTGATCCTCGAGA |
| | | GGCCATCACGAGGGTTGTACTTCAAGAACCACTATCCTTGAGCTACCTAGGAGCTGCAGAATGTGCACTC |
| | | TGCAGGGCTTAGGGCCTGCAGACAAGATAGATGCAGGGTGTCTAGTTAAATTCGAACTTCAGATAAACAA |
| | | CAAATAATTTTTTCAAATAATTGTGTTCTATTCGGTCCCTATTTGGGACATATTTGTACTAAAAAGTATT |
| | | CATTTATCTGAAATTCAGATTCGACTGGGCATCTGGTGCTTTTGTTTGCTAAATCCAAGAGCAAATTTGT |
| | | TCTAGCTACTTCTCAACCCCACCTTCAGAGAGGAAGCCTTGATGGTACTGTAACATCATGCTGTAAGAAG |
| | | GGGATCCCTTGAATTGTAAATGGCACTCTGATAAGATGAGGTATGGGGATTGTATTGGTTTCCTGTTGCT |
| | | GCTGTCATAAATTACCACAAACTTAGTGGCTTCAAACAACACAGATGCATTATCTTACAGTTCTGGAGGT |
| | | CACAAGTCTGAAAGTTAGGGCATCAGCAGGACTGCATTCCTTACTGCGGAGTTCTAGAGAAAAATCCATT |
| | | TTCCTGCCTCCTTCAGCCTCCAGAGACACGCCACATTCTTTGGCTAGTGGTCTGCTTCCATCTCCAAGGC |
| | | CAGTGGGGGCTTATCAAGTCTTTCTCACATCACATGACTCTGTTTCTTCTGCCTCCCTCTTCTACATTTA |
| | | AGGGACCCTTGTGATTACACAGGGGCCCACCTAGAAAAGCCAAAATAATCTCCTTATTTTAAAATCAGCT |
| | | AATCAGTGGCTTTAATCCCATCTGCGATCTTAATTCCTGTCGCCATGTAACACAAGGTATTCCCAGGTTC |
| | | TGTGGGTTAGGACGTGGGTGTCTTTCCTACCACAGGGCAGTTTCTAGTGTTGCCTCTTCTCCCTGCAGTT |
| | | CGCTCATGGAGTCCCGGACCTGGCGCTGAAGGACATCGCCTGCAGCGAGGCCCTCCTGGAGCGCTTCATC |
| | | ATCTTCAGCCAGAGACGCGGGGCAAAGACGGTGCGCTATGCCCTGTGCTCCCTCTCCCAGGGCACCCTAC |
| | | AGTGGATAGAAGACACTCTGTATGCCAACGTGGACTTCTTCAAGCTCTTCCGTGTGGTAAGGGAGGGGTT |
| | | TGGCTGCTCGCCAATTGCAAGGTGATTCCTGGGGTAGCAGAGCCTCACGAATTGACCTTGGGGAGGGCGT |
| | | GAGCCTGGTGTTCTGGACAATCCTTGCAAAAGCTCCAGGCTCCCAGGGCTCAAAAAATCACAACTGATAG |
| | | TATTTCTAGAACAGTGGCCCAGGGACCCAGAAGTCACTATGAGGTTCACCATTAGGTATGTGGCTGTGGC |
| | | ATGTTTGTGTCCACTCTAAATGTGGGGATAATCCCCTTTACCTCCTCTAACAGAGTGGTAAAGGAAGGAG |
| | | GAGGCCTGGTTTGACTCCCTGACCTGCTATTTCCTAGCCAGGTGATCATGGTAAGATATTGAACCTTTTC |
| | | TGGTCCCAGTACTCATCTATAAAACAAATATAATACTTTACAGAGTGGTAGGAATTATACAAGAAAAGTA |
| | | TACGCAAAACATTTCATAAATTTTAATAAATGATGGCCCCATGCTTCTTCCTCTGGAAATGGTCTCAACC |
| | | TCAATGGTTGGTGTTTCTAGAGAGAAAAAACGACAGAGAAAGTTTCATAGTCTCAAAAATTTGGAAAGCC |
| | | CTGATCTAGCTCAACCCTTTGTTCTAGAACTGCATCCCAGACAGACTGCTTGGGACCTGAAAATATCTCC |
| | | TCCTTTGCTAGAAGGATAAGATGAGAAGGAATTAGATAAAGGAGGTGTAGAGCAGAGGTTTTCACACTGC |
| | | AAAGTGCATAAAAACCATCAGAGGGCCGGGCGCAGTGGCTCACGCCTGTAATCCCAGCACTTTGGGAGGC |
| | | CGAGGCGGGCGGATCATGAGGTCAGGAGATAGAGACCATCCTGGCTAACACGGTGAAACCCCGTCTCTAC |
| | | TAAAAAACACACACACACAAAAATTAGCCAGGTGTGGTGGCGGGCGCCTGTAATCCCAGCTACTGAGGAG |
| | | GCTGAGGCCGGAGAATGGCGTGAACCCGGGAGGCGGAGCTTGCAGTGAGCCGAGATTGCGCCACTGCACT |
| | | CCAGCCTGGGTGACAGAGCAAGACTCCGTCTCAATAAAAACAAACAAACAAACAAACCAAAAAAACCCAT |
| | | CAGAGAAGTTGGTAAAAGATGCAAGTGCTAAATCCCCACCCCCAATCACTGTGATTCAGAAGAACCAGGC |
| | | CAGGCCCAGAATCTATCCTGTTACCTTAGGCGATTCTGATGAAGACCATTGTAGGCCACACTTTCAGAAA |
| | | CACTCAAAATTAGAATCCTTCAGAGAAGGTGGCATATATAATATTTCTAGCATGGAATTATGTTTTTTTT |
| | | CTTTTGCCTACATTTTAATTTCTAGAACTGTGTTGTAGGGAATGTCAGTCACTAAGAACTTGATTGAGGA |
| | | ACTGTGTTTTGTCTGTTTCATGACTGCTCTCTCAAGTCCCAGGAAACTCACTTTCAGCTTGTCTTAAAAA |
| | | GCAAGCTGAAGGCTTTTAAAAATGAAGCAACATGAAATAAGACACCGCAGTTTCTGGCACGGTCCACGCT |
| | | TAATCCCCTTCAATGTGTGACTTTCCGTGGAAAGTTACTCTACGATTTTCCCAGCTCGTCAGGGTGGGGC |
| | | CCCAGAGTGAGTATGAAGGGTCAGAGCCTAGGGATGCCACCATCAGTGAGAGCCCAGGACCCCAGAAAAG |
| | | GTCTCTTGGCTCACCACACTGTAGGAAAAATAAAAAGCAATGTAGTCCAAATGTCTCTATCCAAAGTTTC |
| | | AAAAAGAACTTGATTTTAGACACGCTCCTTGACTTGTTTTCAGAATCAGACAGAAGAGTGAGGCAACAAA |
| | | GGTCCCTTATTCCAGGCAGCTGAATACCAGCACAGCCAGGAGTCCAGTGCTGGTGTTTGCAGAGCCACCA |
| | | GAGGCTCCCTCTCAGGTGTCCAGGGCCCGCATGCTTTGTAGAATGGGCAGAATGAGCAATGTCTGTGCAC |
| | | CTGGGCTTTGCAGGCAGGGCCTGGGTACCCAGGTTCGTGCAATCCTCTCGTCACCATGAAGGGAGCAGCA |
| | | TCATTCTTCCCTTCTTGAAGCACCTTGGCCACCAGTATAGGTAAATTTACCTCCCAGGACATGACCATTG |
| | | ATTCTGGGATGTCAATGCCAGAGATAGTAGGGTAAATCGGCACCTGGGTAAAACTTTCCATTGGAGACTA |
| | | GAACCAAAACTCAGGACACTGGCTTCCAAATGTTTCTTTATCAGACAAGAAAGACCAAGTCTTTCCTTAC |
| | | GTCTTCACATGCTGCCTTGGCAAATGCTAGCATTCACAAACCCTGGGCTACCTTGACCTGTCACCCTTGC |
| | | AGACCTCAGACGGGTCCTGGGGGCTTGCTTTCTCGGTTTCTGTATGCAGGCACTCAAACCTGCATCAGGC |
| | | ACCTGTGAAGGGCCGGGCACTGTGCTGAGGCCAAGGCTCCAAATGTGAACCTTCCACCCTCACTGAACTC |
| | | ACAGCCAGACCAGAGACAAGCAAACAGGACATTTCACAGCAGTGCAGCCTAGAAAGGGCCAACACCAGCA |
| | | GCATTTGTCCCCCCGAGCGGTAGCTTTTAGAAGCTTCCCCAGTGATTCAATGTGTCCTACAAATGCCTGG |
| | | CCCCCACTCCCAGAGATTCTGAGTCAGCTGGCCTAGGGTGCAGCCTTGACTTCACTGTGTTAAAAAGCTT |
| | | CCCAGATAAGTCCAATGTCCGGCCAAGATTGAGAATCACTGACCTAGAGTTTAATTTACCACCTCAGTCT |
| | | CTATAGACCACGCATAATAATAGTACCCCACACACCTCTGAGGGTCCAAAGAACTTTCATTTGATCACCC |
| | | ATGAGACCACCGTGGTGTGGAGATGCTTTCTCTCTCCTGTTCTCTTAACAAAGCTGGTGAGCGACAGAGC |
| | | CTGCAGTGGACCGGGAGATGGCCCAGAGGAGAAAGCTCTGCCGTAGTCGGCCTCAGTTAACCACGGAGCA |
| | | CCACCCCTACCTGCTCTCCTCTCACTCCTGCTTCCGTCTCGGTGGAGAAAGATCCAACCGAAGCAGGACA |
| | | CATCTAGTCTTCTGGTGCCTTTAAAATGTACTTTTCCATTTGACAAATGGATTACACTAAAAACAAAAAT |
| | | TTACAAAAAAAAAAAAAAAACCTGAAAGAAATTGCAGGCATTAAAATGGGACTTTGCCTTTATTGCTCCT |
| | | GGGCCCATCCTATTTGGGTTTTTAGAAAAACAAGCCTGAGGCAGGCCCAGAAAGGCTCAGGGCAGACCCT |
| | | CCGATCCTCTGAAAGGAGCATCAGGCAGGCAGGGGTTGCTCCGGGGCCAGGGAAGGGGCCCCGCTGGGAC |
| | | GCGGCTGTTATTGCAGCTGGTTGGCGCGCAGCCATGCTTAGCTGCAGTGCGGGAATGCTGGGCCTTCTGT |
| | | TCTGGGCTGTTTCTCATACGCACGTAGGCCAGTGTATAAATAAGGTTTTATTAAATGCCAAATGAGTTCT |
| | | CATTAACAAAGAAAGAGGGAAAATCTCAGTAAACCACCGTGACGGCATCTACCCACTTTGAGTCAGGAGC |
| | | TGGGGGTGTGAGTGCAACCTCCGAGACAAGGGAACCTGTGGAGCCCAGAGAATCGGAGGGGGGCGCTGGG |
| | | GTTAGCACCGACTGAGACCAGCTGTGTTTTCTCTCGGTTCCTTGGAGATCAGAAGTGAGTGTTGTCATCT |
| | | TCAAACAATCCAAAGGCAGTACCCATGGCCTTACTACATCCCTCCCACACCATCCCACCCATCCCCGCGC |
| | | GTACACTCACACGCTCATTTGCACACTATCGCACACGCTCACTTGCGTGCGCACACACAGATTGGTGACC |
| | | TAGGTGGACTGGGAGAGAAATAAGAGCCAAATGACTGGATTTTCTCCAAGGAAATTTATTAATAGCCCCT |
| | | CTTGGTTTCACCTGAAGGAGCTTGTCTTCACCTGCGGCCTTTGCAGGCTTAACGCCCCCAGCTTGAAACC |
| | | CAGAAGCTCAGACTTGGGCCCAAGGTATTATTAGTGCCAACACTACCTGAAATGTTTCGCACCTCATAAA |
| | | AATGGTGTGTCAGTTTCGGGTGAGAGGTTGGGACGCTTCCCATCTGATTTGGCCCAAGGCATGCATGCCC |
| | | CTCCTTCTCCTTCCCCTCCTCCTCCCCCTCTTCCCCCTACCATCCTTCCTGTTTTCTCTCCAACTCTGGT |
| | | GCACAGCTTTGAAATCTTGCTGAGAAGCAAATCTGTCCCTTCTGCTTTGAATGTTTATTTGTGGAAGTTC |
| | | GGCAGGGGAACCGAGGCGGGTGCCAAGACCTGCCATGCTGCTGGGAAGTCTGAGTCTCCCTCCCTTCCCC |
| | | CTCCTAAATGCTTGTTGATAGAGAAAAGTCAGCCTCCTCGGCATTTGGGCTCACGGTTTTCCTTTGAAAA |
| | | TGCTTCCAGTGTGGCATGATTCAGCTTTCTTTTCTGTCCCCCAACCACTGCTCTGTTGTCATTTTTACTT |
| | | TTCTGATTGCATTTTATCCGTGTCTCTTTGACTACGGGGTGGCTGGACGTTGAGTTCCAGGAAGAAAAGG |
| | | GCCCAATCTTGGGGTTCTGACTACATGCGCCCATCAATGTCCTGTTTCATTCTTGGCTCTGGCTCCCTGA |
| | | ATTCCTGAGTCACTGGGGAGAAGCGTGGGTGGACCGCCCCCTACCCAGTGAGAGTTGCCACAGTTGCTGC |
| | | TCTCCTGGGTCATTGGTTGCAGATTGTTAAACTTCACCTATGCATTTCAACTTTCGGGTGGATATTGCTA |
| | | CGTCAAGTGTCTGGGAAAGCCCCCACAGCTACAGGATTTTACAGTGAGGTCCCACTAATGACTTGATGTC |
| | | ATGACTTCCTCATTCTTTCCAATTTCTCCCACTTCTCCATAAGGGTTTTGGGAAGGGGAGAAGAGAAAGG |
| | | AGTGATTCCTGAGTGCCAGTACCAGGGAACAGCAGGGCTGTTGGGAGGAAACAAAACTAAATCAGGAAGG |
| | | TTTTTGTTGTTGTTTTTGGGGGGTTTTATGAAAATATTCAAGCCACAGCAAATATATTTGATTTATAGCA |
| | | TTAGTATTTTTTCTGCCTGCATCTACAAAAATCTTTACCTATTACCATCAAAATATCCTCTGGGTGAATG |
| | | GATTTCAACAAAGAAGAAATAAAAATGAAATAGAAGAGAGGCCCCTTCGTGCACATTGAGCCTACTGGCT |
| | | GGATTGTCACTTGCCTGCCTTGATGTCTTTTCAGCTCCAGGCAGGCAGTAGGCCAGGGCTTATTTTCATG |
| | | ACAGATCAGATGTTCTTTTATGGATTTACAAAGAAAGAAATACTGAGAAGTCAAAACTGAAGTCACTTAA |
| | | GACAAGAGCAGGCCCCTGGGAAGGCTGCCATTGAGGATAATGAGTCCTGGGGTCCTGGCCTTTGTTCAGT |
| | | AAATACGCACTAGGCGCCTACAATGTGTGCACCAATGTGTGAGGCGTCAGGTTCTCTCCAGGGTCAGTTG |
| | | GTTTTAAGAAAGGTTTTGGCTTCTGATATGTTTTATCTCTACAGAACAGTAGCTCTTAACCTTTCTTATG |
| | | GGTTAGGATTACCTTCGAGAATCTGACTACAGCTCTAGACCTGTTCCCTAAAGAAAACTAAGTTCACAGG |
| | | GACACACAGGATGGGGCTCATGGAGCAGCTGAAGCCAGACCCCAGGTTAATAGCCTTTACATTAAAATGT |
| | | TTTTCTACCTACCACTAATATGCATTCTTTAGTAAGCGGTCTCAATATACACCGATTCTTCCTTAACTCT |
| | | GTTTATGAAGTATTCAGCATCCTCCCTGCCCCCTTCAGCATCCTCCCTGCCCCTGAGCACAGGATCCAAT |
| | | GGCGTGAGGACCACAGGCCTGGGCAGCTGCTGGGGCATACAGGCATCTCTTAGTGGCTGAGAGACTGGGC |
| | | CCTGGCTCTATGTTGGCTCCTAACTTGCTGCCATTTAAAGGAAATCTTAGCCTCCCATCCGTAAAATCGA |
| | | GAAAATAAGACTTGTCCTACACAGCTCATGAAATAGTAATGAAATTCACATTAGAGAAGAGATGGAAAAA |
| | | CACTTTGAACAAAAAGCATTTTGCTCTTATAAAAGCACAGCCTCTTTTGAGAGGCCCTTTGCTCCCCATT |
| | | TCTCCTTCTTCAGACCCCCCCAGACTAGGAGAAGGTCTGTCTCATGGAGTGACCTTTTGGCTGCCTCTAG |
| | | ATTCCAAGCTCAGTTTTGCTTTCATTAACCACAGATACTGGGACGGACAGAAAAAGACCTAGTTTCTGTT |
| | | GAGCCAAAGAGTCTCATAACTTGTCTGTTCACATACCCAAGAGCCCACCCTCTAGTTGAGACACTCAGTT |
| | | CCCTCTCATTCTGGGAGACTGCATGTCTCTGTGACCTCCTGGTAGAGACCGTTTGACATGTCCCCCAACC |
| | | CCCCAGTGATTGAGTCTGAATTCTCCACTGATGACGCATTTCCTAGCACTCAGGGTGTCCCCTCCTGGTT |
| | | GCCCCCTCACCACTGAAGCCCGCTTCCTCCCTTTTCATTTGATGCTTAACAACTGTCAGTTTGCAAGAAA |
| | | CATGCTTCAAATCCACATTCTCCCAGTTGCCTAGCAACAACTTCCCTCCCGGATAAATGTGGGTTTCCTG |
| | | TAGCTCAGCCCAGGACTGAACACAGCAGCACACACTTCTGTCCACTGCTTCAACTGCTTTTCACCTCTGG |
| | | TCTGCATGCCTTCAAGACTGCAGCTCATCCCTCCCTTCAGAACCTTCCATAGCCTGCAGAGGCCATGTCT |
| | | GCCCCAAAAAGACACATTGAACCTGAGGCTACTTATTTACCCTTGTGTTAGGTATATCCTCAACTTAGAA |
| | | ATTAATACTGTTTCCAGATTGTCTTCTTTGAATCACAGAAAGTAAAACAACAAAACATTCAATGCTTAAG |
| | | ACATTTCATGTGCGGTTGGGTGACATCTGTTTGATGAACACATTTGATCCAAAGCATCAGAAATACTATG |
| | | CCAACAAGACTTTTTAGGAGGTGATAAACATGTCTGTTCTACCTTAAGAAAAAAATATTACACAGTCCCA |
| | | AGGGAGAGACATGGTTTTGATCCCAGACAACCCAAGCAGAGACCTCTTAGGGCCGGAATCATCTTGGCTG |
| | | CTGCCTAGGACCTTATATCAATTTCTTAAGCACAGGATCAAGGCCTAAAGGCCCCTTAGACTGACCTCAG |
| | | TTAGTAGAGGCAGATCCCTTCACAGCCTTATCTTCCTTAGAGGTCTAGTCTGACCTTGAACTTCGGCTGG |
| | | CAGTGCTGTCAGTTGTGATGTGTGACATGGAAGAGTTATTTGTTACTTGGAAAATTAAGAGAACTTATTT |
| | | GGCATAGGAAATTGTGTGTGTGTGTGTGTGTGTGTGTGTGTGTGTGTGTGTGTGTGTGTGAGATGATGTT |
| | | TGCCATTTTGATCTGTGACTTTTTTTTCCAGAAATAGTTTCTCAGTTCCATTCCAACTAAACTTACAGTC |
| | | TCTTCCGGTTCTTTGACAGAAACAATTCATGTGAATTTGAACAGATAATAGGGAAGGGGGAACCAAAAGA |
| | | AGAGGAGAGCCCTGGGAAAGTTATTTTATAATTTATGGCAACCTCAGTCAGGCAACTGTGAACAGGTACA |
| | | TATGGAGGGCTCCCTCGGGACTAGGCAGTATTCAGAGATGTAAGGTGTGAGGACCGGACCCTCATCATTT |
| | | ACCATTCCCACTAAAAAGAGCTGGGAAGGAAATTGTAGCTGTAGCACCAGGCACGTAACTGGAGCTTAGT |
| | | AACTATTTGGTGAAGGAATATTATTAAATTATTAACAAGATGGAAAAAAGGGTATTAACCACACAAAAAT |
| | | ACATCTCAAGCTATTGTTTCTCTGTTCCCTTTCCCCCAAATTCCTAGTCTTGCTCTTATCTGGCTGTCTC |
| | | TCTAGTCACTCTTTCTTGCTGACTCTCTTCACGTTCCTTTCTCCACCTGGAATTCCTGGGCCCTCCCCTT |
| | | TTACTGACAGACACTGTCCTCACTCTCACAGTCATCAGTTTGTCTCTTTACAAACCTCAGCTCAAGTGTC |
| | | ACTTCCCCGTCCCCAGGTGAAACTGACTGCTCCCTCCCTGTAAGTCACCATGATGACTGCTATATATAGC |
| | | CCTCATGGAACCTAAAACCTCAACAGACACAGTCTCTTTCCTACTCTGTTATAGTTTATTTACTCATTAA |
| | | TTACCACAACACGTATTATTGAGCACCTACTGTGTACCATGCCCAGAAGATAAAAGACAAACAAAATAAA |
| | | ACCTATTCCTATGCTTAATGAGTTTACAGTCTAGTGGAGAGATAGATACATTAAAAAATAACAGCAAACC |
| | | AAAATAAAAGTGGTAAATAAATGCACTGAGAAAGACAGGAATAGCTAGGAGGGGCACCTAATCCCTAGGG |
| | | AAGGAAAGCTGGAAGAGCATGGTGATGGGGGAAGAAGGCTTTCTGGAGAAGGTGAGGTAGTTTGAAATGA |
| | | GTTGACTCTGGCCAGTAGGGGTAGAGTGAGAATGGGGTGAGACAGGGTGGGTTGGTCATTTTGATCCATT |
| | | AGTCCTCAAAGTGATAGGACTAGTGGCTAAGGACTGCAGGCTTTACAGAAGCCTACAAAACTATTTGAGA |
| | | TTTGAAGTTTTTTTTTTTTTTTAATTGGCTCCAAAAGAAAATGAAAAAACTTTAGAATTATAATGAATGA |
| | | ATATTAAATGAATATTTAAGGAAGGTAATTTTATTCAACTTCATTGTTAAATTTAGTTAAAACAAGCCCT |
| | | TGAGTTTCATTCAACACTGTTTTATCATACCGTTGATGAGAGAAAACAAAACTGATTCCTGGCCAGGGCC |
| | | ACTGTCAGCGTGGGGTTTGCACATCTTTCCCATGTCTGCTTGGGTTAGCTCCAGGTACTCCTGTTTCCCC |
| | | CACATCCCCAAGATGTGCCCATTAGTGGAAACGGTGTGTCTGCATGATTCCAACGTGAGTGAGTGTGGGT |
| | | GTGGGAGTGAGTGCCCCTGCCATGGGAGGGCATCCTGTCCAGGTTAGATTCCTACCTTGTGCCCTGAGCT |
| | | GCTGGGATGGAATCCAGCCACCCATGACTCTGAACTGAAATAATTGGGTGAATAATTATCTTACTTTTTA |
| | | ATTAATCTTTGAAAATGTATGTATAGTTCACATGTATTTCAATATTTAATATTAGAAGTATTTTAGTCTT |
| | | TATTTTGAAGTTTGGTGATTTATTGTAACCAGAAACAAGCTATAGAAACTTAATTTTGGGCCAAGTGCAG |
| | | TGGCTCACACCTATAATCCCAGCATTTTGGGAGGCCGAGGCAGACGCATCACTTGAGGTCCGGAGTTCAA |
| | | GATCAGCCTGGCCAACATGGTAAAACCCTGTCTCTACTAAAAAATACAAAAATTAGCCAGATGTGGTGGG |
| | | CACCTGTAGTCCCAGCTACTTGGGTGGCTGAAGCAGGAGAATCACTTGAACCCGGGAGGCGGAGCAGTGA |
| | | GCAGAGATCGTGCCACTGCACTCCCACCTAGGCGACAGTGTGACACTCCATCTCAAAAAAAAAAAAAAAT |
| | | AGAAAAGAAAGAAACTTAATTCTGGTTTATATCAATTAGCCTGTGGTAAAATTGGTTTCATTATAGCCAT |
| | | TTCACTTAGTTGAAGTTTCCAATAACCTGTGGATGAATTAAGTGAGGATTTACTATATTCATAAAATCTT |
| | | AAATTCCAAAGCCTGTTTGCAGTTCAGGTTTTTCCACTTTACAAACACTTCTAAGTATTCACAATGATTG |
| | | CTTAAAATTCATACCAGATAAATCATTAAATAAGTTGTTCAAAGTCAAATAATTTCATAAGTAAAAATTA |
| | | GGAGCTTTTAGAAAACTATACCTACATAGACCTAGACCTATAGATAGACAGAGATCTGAATAGATATGGA |
| | | CACAGATGCTTTCCAAAGTGTTCATGTGATGTGTGGTGGAGTTTCAAGACCAGAGTGTGCCTGGGGCCTG |
| | | CAGAAGTAAAGGAGAGGGGATGGAGAGAAGATTGTCCACATGGCCATGGGCAATCTCCCACCCACACTCA |
| | | AGTGAGGAAGACAGGAAACAAATTCAGAAAGAAGAGAAAATAATCAAAACTGATGGGAGCTTGTGACTGA |
| | | TTTACTTATGCGCAGCCTCCCTGGAGACATGAGTGTGGCTGTTCCTTAGGTTGTGCCTCTGGGCTCCTAC |
| | | CCCCTCTTAGATGCCTTCCTATTATCTAGGACCTGGTTGCTTTTTGTCTGCATAGCTTCTTTGGATTCCA |
| | | GTCTTTGATGCCAGCTTCCTCCTAAAGTAGCCTTTCAGATGTCCCTTGGTTACCCTCTGCTATCTAAGGG |
| | | CTCATCCTACCCCACACTCATTCCCAGCACCAATTTCTGGATCTCCAGGCTGGAGATTTAGACAATGGGA |
| | | TGGGAAGAACCCATGATGGGTCCCAGACAGAAAGTGGTGCCAGCCACAGAAAGGGCACACAGGCACAGAA |
| | | GTTGGTTTGGGGTAAGACGATGTGGTCAGTTCAGAACACGCTGGATCTAGGCAGATGCCCAGCAGACAGT |
| | | TGGATATGTAAGTCTGAAGCTCTGGGGAGAGGTCTAGGTTGGAGGTACAGATTTAGAAGTCATCAACAAA |
| | | AAGGTAGCAGATTAAATGATAAAGGAAATGAGACTATCCGGGGAGTGTGCAGAGTGAGAGGAGCAAGGGA |
| | | GGCCCTTGGGAACCTCAGCACTTCAGGGGAAGGTAGAGGTACAGTTGCTGGTGGGAAAGGCAGAGAAGTA |
| | | GCAAAGCAAACCAGGCAAAAGCAGTGTCACAGACGACCAGGGAGGAAAAGGACATGATCAAAATGTTGAG |
| | | AAAAGCAGAGAGGTTTGAAAATACAAGAAGCAAAAATGTCCACTAGACTTAAAAACCAGGAGAAAACTGG |
| | | GGGGTTCTTGATAAAGCATCTTAGTAGGATGGTGAGGGTAGAAGCCAGGGAAGTGTTGGTGAGGAAGTGA |
| | | AGTCACTGATTACGGACTATGCTTAAAAGAATGTGGGAATGAAGGGTGGAAGAGAGAAATTAGACTGTAG |
| | | CTAGGGAGACATAAGCGATCAGAGGTAGATTCTTTCTCTCCTGTGGGAGAATCTTGCACGTATACACAGC |
| | | ATGACGACAGTGATGGAAGGGCTGGCGAAGCCTCAGGGAGACTCTTGGAGGTAAACCCCATGAAGGGAGG |
| | | ACTTTGTTTCATTCACTGCCGTGTCCCCAGCACCTGGCACAATAGCAGACACTCAATACATATTTGTCAA |
| | | ATGTGGGATTTTATCATTTAGAAACTGCACCTGGCTGTGAGTAACAAAAGTCAGAGAAACCGTGGGTTTC |
| | | ATTTTTCTCCCCAGGCAGAGTCTGGAGCTGGGTCCTCCAAGAGGGGTTTGGAGCACCACAGGTTTCCTCA |
| | | AGACCCCCAGGCTGCCCTGTGTTTCCCTCCTTCATCCCCAGCATATGCCTGTCATCTGGTGACCTCCAAA |
| | | CACCTGTGCTGCCTCCTCCAGCACATCCATGTTGCAGGCAGGGACCAGGCAAAGGGCAGAGGGGCCTACT |
| | | TCAAAAGACCATTTCCAGAAACCCCATCCTATGACTTCTCCTGGTGTCTTGGTTACCATTGTGCCATAGG |
| | | CTCACCCTGTATGCATGGGAGGCTGGGCCAGGCATTATGACTTTTAGCAATATTGCATAGATAAGCATCA |
| | | ATCTTTGTCACTGTGACGAAGCCTAGTCACTCAGTGCTAGGCAAGGTTAATGGAATGGGTTGGTGTGTGC |
| | | ATTATTCTTGAGGTCTTTCTTATGCTTCATGTTATACATTTATTAGGACGTTTAGGCAACAGGGGGATAA |
| | | AAATGAAGAGGAGATGCATGCTATGATCTGAATGTTTGCATCCTCCCCAAAATTCATATGTTGAAATCTT |
| | | CATCCCCAAGATGATGGCATTAGGAGGTGGGGCCTTTCGGAGGCAATTAGGTCATGACTGGGATTAGTGC |
| | | CCTTGTAAAACCCCAGAAAGCCAGCTTGCCGCTTCCACCATATGAAGACACAGAGAGAAGATGCCATCTA |
| | | CGAATCAGGAAATGAGCCCGCACCATGCAATAAACCTGCTGGAGCCTTGATCTTAGACTTCCCAGCTGCC |
| | | AGATCTGTGGGAAATAGATTTCTGTTGTTTACCCAGCTTATGGTATTTTGTTGTAGCAGCCAGAGTGAAC |
| | | TAAGACAGTGCTGATCTCGTATTCTTGGAGGGAACCCTTAGTCTTTAGGGAAAGCAAAGCCACCATTTGG |
| | | GGCAGGGTGTTCTCCAAGTGCTGCCACATATGCTGATGTGGTTAAACTGCAAACTATGGTAAAAATGTGG |
| | | AGGTCTGTGGAATTGTCAATCAGGAAAAAGATATAAAAAGAAGTTAAAGTCTTCGTGCTTCTGGAAGGAT |
| | | ATGTGCCAAATTGTTAACATTGATTATCCTTGGGTAGAGATGTGGGGAAGTTTGCAGAGACAGTTTTGCC |
| | | TTGTACTTTATATAAGTAACAGCTACTACTTCGTTGTCTTAAAAAAAAAAAAACAGCCTATGTGCTCTTC |
| | | ATGTGACTCAGAACTACCTAGGCAATACGATTAATTGAATTAGTAAAATTGAGTGATTATGAATTTTCAG |
| | | GAAGTCATTAATTTACCACTTCTTTATTACATCCACTTCTAACAGGACTTCAATATAGGGGAATTTGACT |
| | | TCAAGATAAAAAGACCAAATTTATTTACCCTTTTAAAAAAAGACAACTTAAAAGCAGACTTGTCTTACAG |
| | | AACCTTCCTTAGTTGGACATCGATGAGTGTACAGAAAATGCAATGGATAAAAAGCTTGGTGATACAAAGA |
| | | TAAAAAGTGGGGTCCTGTCCTTAATGAACATACCATTTCATGGAGTATCAGGTGTATAAACAATTATAAT |
| | | CAATCTGCTTGTTATTCTGATAAGATCATTTACTCACACATCAAATACTGAGTGCCCACCACATGCCCAG |
| | | CATACCTAGAAGTCATCCAGTATGATTTCTGTCTACATGGAGCATAGAGTCTTACAGGGGAGATAGATGA |
| | | CAAGTAAACACCAGAATAATTACCAATGGTGAAGAGCACAAGGAAGGAAACAGAACTCCTAAAGAGAGCG |
| | | TGGCTGGGCAGGGGTGAGCAAGAGGCATAGAAAAAGGGGCATCTAAATCTACTTGGGAGGAAGCTGTTTC |
| | | TCACATAGGTCATCATGTTAGGAATGAGACTTGAGGGATGAGTAGAAGTTTGCCAGGCAAAGAAGGAATG |
| | | GGGGGGAATAGAGAGCAGAGCTAGGGGCAGGAGACAGCTGACGTGTGAGCAGACATAAAAAGAAGTCCAC |
| | | TGTGGCAGCAGAGAAGCAGGAGAGAAGGCAAGTGAGGGAGCCAGGCACCAGCTCACAGAGGTCATGTGTG |
| | | TCAAAACGTAGTAATGGCCTTCTCTTCTGGAGACAGTAGGGAGCCATGGAAGATGTTTGAGCAGGGAAAG |
| | | CGACATGACTGGATTGGCCTGTTGGGTAACTCAGACCACAATGCATTGGAAGGGAGGGGGCTAGAGGCAA |
| | | GGGGACTGGCAAGAAGGCCAGTCCTTTTTCTATGCCTATTTTGATGAAATATTCTAGAAGGGAAGTGAAC |
| | | AAAGGTAGTCCTAGAGAGGAAGAACAAAACAGATAGGATACTTCCTTAGTATTTGCTCATTCGACAATTT |
| | | ATTTTTGCATATACACTAAAACCTTTTTTATTATTAAAACGTTTTATTGTAGGAAAAAAGTATGAAAGTA |
| | | GAGTGAATAATAAAATGAGCTCCCATGGATCTATCACCCAGCTTCAACTATTATCAATATTTGGCTGTTC |
| | | TTGTTTTAACTGTTCTCCACCTTTTTTTCCTGAAGTTTTTTTGAAGCAAATCACAGACAACATATCATTT |
| | | CACCATATGTACTTCCCTCTGTATCTCTAACATGTAAGAACTTGTTTTAACAAAATCACCATGCTATGAT |
| | | CATACCCAACAAAATTTATCATAATGTCTTAATAATACCTAATACCCATTTCATGTCCACTTTCCCCCAA |
| | | TTGCTACAGCTGGTTTGTTCAGATCAGAATCAAAATCCACCTGTGGCCATTTTACTGCTATGTCTCTCAG |
| | | GTCTCTTTTCATCTCTAATAATCTCAGGGGAGACAGGAGGGAGGACGGGCAGGACTTGGGGCTAACTTGC |
| | | TTATCGACACACAGTTTTGCCTACTTGCTTCCTCCCTTCACACCCACTCTTCTTCTCAGCCCCACCCTTG |
| | | TATGGAAAAAACAGAAATTAAAGTGCTTTGCCCAGCACCCACTGAAGCTATTTCGAAGGAGTTTGAAGAG |
| | | TACTCCCGGCAAGACAAATGCCTCGGTCCAGTGCTCAGGTCAAAGAGGGGAGACGCTTCTCAGTGATGTG |
| | | GTGTCAATAGCAGCTTAGTTGTTCTTTCCTCTGGAAAATTCTACCCATCTGCTTTGTAACTCCCATACCT |
| | | AACAAGGCCTTTTATTTCACAATTAGAAAATAAGCCTGAAATATGAATGCTGCCTGAGTGTACCTACATT |
| | | TATTCTAGAGTTTCAGGGTCAAAAAGAATACAAGGACCTCTGCATCTACAGCCAAGAGGAGAGGGGCAAA |
| | | GACACACAGCTACAAATGAGAACCTGGCTGGTCAAAGCCTAACTCCACCTGTTTGTCAGCACTGATGCAA |
| | | GTTAGGTCAGCCCAATGATCATTTAGGAGAACTGTGCTGGCAAATAAAAAGCAGAGGCTTTTGGTCCCCA |
| | | GATACTTGGATGAGAATTACAAGTCCAGCTGGTTAAAAGGCACATGCCCAGTGCTCACTTCACACCTACT |
| | | CAGGAAGCACACTTGAGTTGGAAAACCACTGTCTTTACACTTAGAACTCAGTCCTACATGACTCCTCTAG |
| | | GATCAGTGATTCCATCAGTTTTGAAACATGAAGCATGAAGTCAAACAGGACATGACCTTGGTTTCCAGAA |
| | | AACCAGATGTTCACATCAGTCTCTGGAGCTTGGAGGCAGCACACCTGGGGACTTCCACATCCCCTGCCGA |
| | | GGTGGCAAAAGCAGGAGCAGTGGTGAGTTCACATGGGCTGGGGTTTCCTGAACACTGCTGGCAATTGGAG |
| | | AATCTGCAAGGGAACTTCTCCGACTCCTACCAGCAGCTGCTTTAAAATAAAGGTGATGTAGCTGGTCAAA |
| | | TCCTCCATGAGAGAGCAGTGTTGAATGGAGGAAGAGACACAACCTGTCTGAAAATGGCACAAAGGAAGAA |
| | | AGATGTAAACAATGACGAGAAGACTGCAGTGTCTACAAAGCTCCGAGGTGAACAGATGGGCACCCCAGGC |
| | | CCGCAGCACTTCCTTCAGTCTCTGCCAGCTGCACTCTGTTTTCCTTCCTCCAGGAATCTTGTTTGGTGTC |
| | | ACTAAAACAGCAATTAGAATCACTTTGAAATAGTGATAGTATTTAATATAACTATGAAACTATCTGTGAT |
| | | TGACAAGTGCAGCAAGGAGTCTTGGAATGAGAGCCTTTATTTTTTCAATTAAATAAAAGAGTTTTTTGTT |
| | | TCTAAAAGTAATCTTGCAGAAAAGATCCTGCGATCAGAAAGAAGGAGGGGGGGAGTTTTCAAACATATAG |
| | | GAGATCAGACTGTGCCTATGTGTGTATATACCTACAAACATATATATATTTAAAAAATTGTTTTACTGTC |
| | | AATTACAGCTTCCCACACTCCTAGACAGCCGTTCTCAAGGTATCAATCTGAGATCTTGGGGAGGAATATT |
| | | ATCTGATATGTCACCAAGAATTCAAGAGGTGAGTAGCCTGATGGTAGTAATTATAATTTCATTATGTCTT |
| | | TCCACCATTTACCCCACTTATGTCAAATAATTTAATTGTATTTCAAACCTGTTCAAGGAAAAGTACATTT |
| | | GATCTTTCCATCTAGCAATTTCAAAGCACCTGTTCACATCCCAAATTATCTGTGCTCTTAAGTAAGAGGC |
| | | AGAAAGAAAGGAACCACCCTTCTGATTTCACATCAAAAAAGAAATGCCACTGGCAATAAGCAACTTGCCT |
| | | GGTGTGGCATAAATCATCAGAAGACTTACAGTTGAATCTAAGTCTTTTCAGTACTGAGGTGGTTCATTAT |
| | | TCTGTTACAGTCTTAAAATTCACATAAATATATACTGCCAATAATAATAGCATACACCTTTATAGCTTAC |
| | | AGGCACTCTTCTTCTAAGTGTTTTACCTATGTTGGCTTATTTCATCATAAAGAAAACAATGGACTTTTGT |
| | | GTTGTTTTGTAAAAAGATGCGCACATTTTAATTAACATCTGATTGCACAAGTCTCCTCCCATATAGAAAT |
| | | GGATTCTTCCACGCAATAGATAAGAGGTGCTGGGGATATGATGATGAACACACAGATTTGGTCATGACCC |
| | | TGTGGGAAAGAGAGATGGGAAAAAAACAATTCTCTTCAAGTGTGATGAGTGTTACGAAAGGGAGGGAAAA |
| | | GTTGAAACAGGTTTTTTTCCAAACTTTTCTCCCTCCATTATTCGCAGCTGACTTGGGCTCCACCAACCTG |
| | | GAAAACTGCATGGTTGGAATCTGTCTTTATAAAACGCATCTCAACCTGGGCCGAGTATGCACACTGATGT |
| | | GGGAAAGTTAGAGAAGAGCCCATTGTACTAATGCTCACCTGCTACAGTGGGAGTCTCTGTTAAACAGTCT |
| | | TTTCTTCATAGCATTAAAAAAATTTATATCACTACAATAAGGTTGAAATTGATAGAGAATGTACAAACAA |
| | | TCCCCAAAGTATATCAACACTCTTAGTTCTGAGTAGAAGTTCCAGAAGGCTTCTTGACTGTCTAGATAGC |
| | | AAGTCTAATCATTTGTGAACTAAGTTAAAGCAGAAGGCCCAGTTTATATGAATTGGTATTACACCATTTG |
| | | ACCTGAGAACAGCCCCTTCATCTCTGAGTGCTTTGACTAAATGAGCAACATAATAATAGTAATAACCCCT |
| | | TACAAGATGTCATAAGACTCACTGTTGTTGAAGCAATTTGAGATTTTGACTTTATTGAAGCATAGATGGT |
| | | GATTATAGGCATGACTCACTGTGTGGATTCTCCCTGGGCTCATCAGTTTCAGAGGGCAAGTGTTGGCATG |
| | | TGGACAAAGAGAGGGATGACACGTAAACATGGCTTATTGCAATGGGGAAATATTTTCAGTCTCACTGATT |
| | | GAATCCTAATGGTTTTATAAATTCCCCAGTACCACTGAAAGCAAAGCAAGTAATCAGGTGTGTTTTAGGA |
| | | ATAAAAGCAGCATTATTTTAATTTCGTATTTTCCCCTAAAGCAAAGCCAAATGGCATTATGGGAGCCAAG |
| | | CTACTGGCAGCTCCACCAGCCTTCTCCTGAGTTCTCGGCATTACAGATCTACCCTCAAAGGATGAGGCCA |
| | | GCAAGCACCACAGGGTGCCCACATGGAGAAGAGAAGGCCACCAACCTCCTCTTAGCTGGCACAGAATTGA |
| | | AAAAGTGTTTTTCCAGGAATGGATACTTCATCTGTTCTGTATTTGCTAGAATTTTAAAACGCACACACAG |
| | | ACACACACAGGCGTGCACACACACACGCACACACACACGAGAAAACCACAAACCACACATTTCAAGGAAA |
| | | TGGAAGAATTCATTGGTAAAATTAAGCTAATAAGATTATTTTCCAAATATAAGAAACTAAATTTTAGACT |
| | | ATTTAGCCAAAGAAATTTGCTCTGATCTTGCTTTTCTACAACAGAATCATTCCCCAATCATTTTATTTCC |
| | | CTCTTTTTCTCCCCAGTATCCCCATCTTGGTGGGACAACAGAACCCAAGAACTGGCTTAACAGTAAAATA |
| | | TTTTCTGCATTTGCCCAAGGACACATTCCCAACGAATTCAAATAAAGGAGACTAGAAGAAGAGAGGCTAT |
| | | ACTACAGTGCTCTAGGGGTCACTCTGTGATTTGTTGTTGTTGTTGTTGTTGTTTTGAGACGGAGTATTGC |
| | | TCAGTCGCCCAGGCTGGAGTGCAGTGGCACGATGTCTACTCACTGTAAGCTCTGCCCCCCAGGTTCACGC |
| | | CATTCTCCTGCCTCAGCCTCCCGAATAGCTGGGAGTACAGGGGCCCGCCACCATGTCCGGCTAATTTTTT |
| | | TGTATTTTTAATAGAGACGGGGTTTCACCATGTTCGCCAGGATGGTCTCGATCTCCTGACCTCGTGATCC |
| | | GCCCGCCTCGGCCTCCCAAAGTGCTAGGATTACAGGCATGAGCCACTGCGCCCGGCCACTCTGTGATTTT |
| | | CTTTAAGGCTCATCCTAGTATTCTCCTAGTCCCTAAGTAGATGGCAGTAGGTTTTGTTTTTTGTTTTTCG |
| | | CAGCTGGATTAAGGATTGCTGAGAATATATGGATGTTTTCTTTTAAATGTGGAAGTCAAACCAAACGTTG |
| | | GAGCATTGGCCTCACAGCAGATTATGACTCTAGCTGCCTTAAAATAACCTGAAGACTTTGCCTTGCCCTA |
| | | GTTTATCCATCGGCCGAGTATGCAGGACTTGCTGTGGGTGACCAGGCCCCTCATGCAGAATGGTGGTCCA |
| | | GAGACCTTTACAAAGCTGATGGGCATCCTGTCTGACCTCCTGTGTGGCTACCCCGAGGGAGGTGGCTCTC |
| | | GGGTGCTCTCCTTCAACTGGTATGAAGACAATAACTATAAGGCCTTTCTGGGGATTGACTCCACAAGGAA |
| | | GGATCCTATCTATTCTTATGACAGAAGAACAAGTAAGTTTTCTGAGTCCTGCTTATAAATTGGCCTCTCA |
| | | TGTTGGTTAAGTTGATGGTTTAACACTTCTAGGTGAAACCAAACCTGGGGTTGCATCTGTCTTGTCTTGC |
| | | TGAGTGGCCTTAGGTAAAGAGACTTCTCCCAGAAAGTCCACTTCCCCTTGCAGAAAGGGGGCATTGCTTA |
| | | TAAGCAATTCTGGACATGAACCACAGAAAGAACTGAGGCCCACTTGGAAAGGGAACAGAGGGGCCATTTC |
| | | CCACTGATGTAATTGAACTAGGGCTAAGTTCAAGAGGAAGAGAATGATCCGCAAGGAAGCAACCCAGAGT |
| | | TCCAGGTGAAGCTCAGGTCAGAAGGGCCCTGGCAAGTAAACACGGCTGTGGGATGCTTTTACAAACACAA |
| | | TATCGTGAAAATCTATGTGTGTAGTACTGAATTACATTCCAAATGGCAAATTCCTGGCAAATCATCTTCC |
| | | CCACCTTTCACTATTTTTTTTTTTTTGGTCTTCTATGGGGTAAAGGAGGATGGGGTGGGGAAGAAATGTA |
| | | ACTGGCTGCCCCTCTAGTTAAAAACTGAAAAGAGGCAGCAAGGGACATGCCAAAAGTAGTTGGACTCTAA |
| | | GATAGCTACACACAACAAAGCAGCTAAGCAGCTAATTGAAGGGAAATTACTGAGGCTCAAGCTGAGATTC |
| | | CAAGCGGGGGCCTTGTTTGGCCTCTCAGTCCCTTTCATCTGAGAAAGGCCTCAGTTCCTAGCAGTAATCA |
| | | GAGGCAGGCTTCTCAGCCTCCTTCTCCTAAAGCAGAATAAACCACAGGGCAAGTCGCATCCTTTGTTTCT |
| | | CTGATGAGGCCATTACTGAGAGTCACTGTGGCATTTTGCTACTAATGATGAGCTTGTTATTGGTGGGGTA |
| | | CAGCCTATTAATTTAGGTTATTCATCAAATCCTCCAGCATGGAGTTGAATGAGACATGTGATGTGGATAC |
| | | ACTAATGACTATATTGAGTTACAAGCAATGGGGAGTTTCTGTAAAATCTGTCCCTTGTCTCCTGGCAGCA |
| | | TCCTTTTGTAATGCATTGATCCAGAGCCTGGAGTCAAATCCTTTAACCAAAATCGCTTGGAGGGCGGCAA |
| | | AGCCTTTGCTGATGGGAAAAATCCTGTACACTCCTGATTCACCTGCAGCACGAAGGATACTGAAGAATGT |
| | | AAGATCCCAGCTGGGCTTGCCTTGTGTACCCTGGACCTCCCAGAAGTGTGTGTGTGTGTGTGTGTGTGTG |
| | | AGAGAGATGTGCCTTCCTGGTAGCACATCTCATGTTTGTTTTTTGCTAAGTGGACTCTTGCGTTTCCTCC |
| | | CCCATCCACAGTCATCACTGGAATGCTTTGCTTCAGTGCCCCTGCCTGGGCCCTCCCCTCTCTACTGCAG |
| | | CCTACAATGAGGTTTTCTTTCCCATTGCTTGAATTATATCCCTAATGGAAGGGTTCACAATTCTCTGAAT |
| | | CCTGGCTACTCAGATAAAGACAGGGAGGAAGGGAGGAAGGGTATTTTCTCCCAGGGGGTCCAAATCTAGC |
| | | TTTAACGAGGGAGGTTCTGAGAAAATAATATCATCAATATTACATGGACTTCTGAGATACTAAGAAATTA |
| | | GATTCTGTCAGCCCAGGAAGTTGGGAGATGGTGAATTGTTCTGGGAAATAGCAATAGACTGAGAAAATAA |
| | | AAACACTTCCTTGAAAAGCCTTTCCCTAACACTAAGTGATAGGGGCAGAAAAGACACAACCAAAAGTTCT |
| | | CTCTCACTTTTCTCTCTGTTCGTGTCTCTGTCTTGATCTCTGTCTGGTTTTAGGCCAACTCAACTTTTGA |
| | | AGAACTGGAACACGTTAGGAAGTTGGTCAAAGCCTGGGAAGAAGTAGGGCCCCAGATCTGGTACTTCTTT |
| | | GACAACAGCACACAGATGAACATGATCAGAGTAAGGGGGGTTGGAGGATGGGGAGGGGAGGGGAGGAGGA |
| | | AGCGGTGGGGGCAAGAAAGTTCCACTTGTTTCCTTTTCCCAGGAAAGAGTTAATCGCTATTGGAGTTAGA |
| | | TCAAAATACAACAAGCAGGCCCCAAAGGCCTTCATTCCAAGCAGTCACCAAGTGGGGTCACTGACTTTGG |
| | | ATGAGAAATATGTTTCTTGAATTCTGGGAGAAGTCTAAAAGCTGCCACAAGACCAGTGGCTTCCTGGAGT |
| | | TTCCTACTTTTATGAATTCACTCAAGGGCCTCAAATTCAAAGAGGCATCTCCCCAAGGGGCCAGCTCTGT |
| | | AACTCCAAAGATGGTGGAATGTGTTTGTCTGGTCTCATTTTCAGCTTTGCAAAATGAAGACAAGAGTTCT |
| | | ATATATCAGGGACACTCAAAAGAAAACAAAAATATCCATAAGCAAAAGAAAGCTTTTTATACACCATATT |
| | | CAATGACCCCCATCTGGCCCCTCCTTTGCCCCTACACATCTTCCCTCTATTCTAGAGACCCATGGACTTG |
| | | GGGAAATGGGATATAGATAGGTATGTTTCATAGTGGAACAAGCTCACCAGCTCTTCAGGGAGCCTTAGCA |
| | | TCTCTATCCTCAATCACTAAAAATTAGAAATGGCTGAAGAACAAGACCAAAGATCCTATGGAATTTCTAA |
| | | GCAGAGCAGTGACTGTATTTCTTCTTCCCAAGGATACCCTGGGGAACCCAACAGTAAAAGACTTTTTGAA |
| | | TAGGCAGCTTGGTGAAGAAGGTATTACTGCTGAAGCCATCCTAAACTTCCTCTACAAGGGCCCTCGGGAA |
| | | AGCCAGGCTGACGACATGGCCAACTTCGACTGGAGGGACATATTTAACATCACTGATCGCACCCTCCGCC |
| | | TGGTCAATCAATACCTGGAGGTAAGGGGCTGCAAGCCCCACAGTGGGCCCCTTGAAGATAGCCCCATGAG |
| | | TGGGGCCAGAGCTCCCTTAGCAAGTCAAGTGGTCTTGAATTTAAGCTTTCATTTTCCCCACTGAAGAAAC |
| | | AAGAATCCCTACATCCCCTGTACAGTTCTCATTCTCTAACAGCTTATCCATACTTAAAACTTATCTATGC |
| | | TGAAAACGGTTTCCTCTTCACATCTCCTACTTCTCATGCTGGGCACCTCCTCCTGTAGCCCCCTTTAAGC |
| | | ATCTGTGTCTGTCCTCAACCCTCTTCTGTCTGACATTGCTTGAGTGGCCATCTATGGCCAGTGTCCCCTC |
| | | AACCCCACAGTCCATTGCTTGCTGGACACTCCTGCCCTCAAGTTCTACAAGCACATCAGCCTCAACATGT |
| | | CCCCTCCAAAAACTGTATGTTCTCCTTGCCCATAGAACATATCCTTCTCCTATATTTCCTATCCTAATTA |
| | | ACGTCCTCAGCATTTGCCCGAATTCTCAAGTGAGGGATTTCAGGGTCATCCCTAATTTTCCTTCTTCACC |
| | | CTCCACACAGTAGCTGTCACTTACTGAGTGTTACTTTATGCCAAGTACTGTGCCAACTGCTTTTACACAC |
| | | ATATGCTTCATTTAATTCTCACAGCTCCATGAGGCTTGCACCATTATCATTGCCAATTTGCAGATGAGAA |
| | | GCCAGGGCTTAAAGAGGTTAAATAAGATCCCACGCATGACCATTAAGAGGAGCGAACAGGATCCAGCTCT |
| | | GGGGGTGCCTGAGTTCAGAGCCTGCCTTTCTGATTTCTCTTACCAAGCTTTGTCTCCTCTCCCTCCTAAA |
| | | TATCTCTCAACTCTGCCTCTTGCATTCCAGGCTCTCTGAGGACTAGAGGCCTTGTCATCTCTGCGCCAGC |
| | | CCATTCCAAGGGCTTCCTTCCTGGAATCCAGGGTCCAGCCTCTGTTGGCCCAGGCATTTCTCTACACTGG |
| | | CACCAGAGTTACATTCCGCACACCTGCTTACGTTGCTCTCTCACTTAAAATCTTAATGACTCGACCCCCA |
| | | AATAACACAGGTCCCTTCCAAATCTGTCCTACCCCACCTTCCCAGCCCTTGCTCAACTCTGCTACCTGGC |
| | | CCTTTCACGCCTACAGGCATTCCCATTCCATGACCTCTTGGGATTCTACCCTTTGCAAATGCTGTTTTCA |
| | | TTGCCCATTTATTAGAGCGCTTTTGGTCACAAGCTTTTTGCTTAACCAAAAAGAAAGCATTTATTGGTGG |
| | | ACATAAATAATGAAGTTCAGGAGGATCCAAGAGTTGGAAGCCACCATGAGACCCCTGTGTCCTTCCACCT |
| | | CACTTTTCTACTCGCCTCTGCTCAGCTTCATCTCTGGCCAGGCCCTCTCCTCTGCTGATGCCCTAGCTGC |
| | | TTACAGCCCTTAGCAGTCATCTATACACCAAAAATCCCTTTCCCATAGCAGAAGCAATGCTCCTAGAGAG |
| | | TTTCCCTGTTGGTCTGGCTCCTGTACCCACCCCTGTGTACTCTGATTGGGAGGCCTGGGTCAGCTGCCCA |
| | | CCATGGGGCCATTTCTATGAGCAGGATTACTGTGAAGTGGAGGAAGATGTTTCCCCAAAAGAAGAAACAC |
| | | AAGGTAGAAAAGTGTATGTCCACCAATGCCTGAAATGACTGTCCCTTTCCTCATCTGCTGAGCTTCTACT |
| | | CATTCATTCTTTGAGACTCAGCACTCAGCTCTTAAATGTCACTTCTGCTTTGATGGAGGTTTAGTCATTC |
| | | ACTCCTCTGTGCTTCCTGGCCCTCTCTTCACACCTCTCTCAGACCCCTCTCCCAGATAGATTAGAGTTGG |
| | | CTGTTGACATGTCCATCTCTGGCTGGGCAGCTAAACTGGAGTTATTTAGAATCAGGGAGCACATGTCAGT |
| | | CATTTTCAAATTCTCAACCTCATACTCCCAGTAAATGACTCCATCTAAGGGTGGACCACTCTTGCCCATG |
| | | GGCCAGGTCTGGGTCTGTGTCATCTAGAACTGTTGGAAGGTAGGGGCTTCTGTGAGCAGTAGGAGAGGGA |
| | | ATAAACTCGAGGGCCCTCGGGAGCATGCCCTCTTGTCTCAGACTTGTGAGTCCTGAGGATAACAAACTAG |
| | | TGAAGAAAAGCCTCGTTCTATCTGTCACCTGGTGCTCTTGAGGACTTTCTGTTGCCCTGGTGCCACCACA |
| | | ATTTTCCAGAGTGTGTGACCCTCGCTCTCCAAACTCTGGAAGTGGCAGCCGAGGCTCCCCAGTGGCCTTT |
| | | CAGAAGGTGCCAGTCATGACAGCAGCACCAAACTGCAGGCAACTACTAAGCGATCACCAACTTGTCTGAA |
| | | GATAAGAATGACCTTGAATGCATTTTATAAAACAGGATTTTTTTTTTAATTTTTAGATTTTCTTTCTTTA |
| | | TTTTACCTTAAGTTCTGGGATACAAGTGCAGAATGTGTAGGTTTGTTACATAGGTATATGTGTGCCATGG |
| | | TGGTTTGCTGCACTTGTCAACCCATCATCTAGGTTTTAAGCCCCACATGCATTAGCTATTTGTCCTAATG |
| | | CTCTCCCTCGCCTCGCCCCTACCCCACCCCAACAGGCTCCGGTGTGTGATGTTCCCCTCCCTGTGTCCAT |
| | | GTGTTCTCATTGTTCAGCTTCCACTTACAAGTGAGAACATGTGGTGTTTAGTTTTCTGTTCCTGTGTTAG |
| | | TTTGCTGAGGATGATGGCTTCCAGCTTCTTCCATGTCCCTGCAAAGGACATGATCTCATTCCTTTTTATG |
| | | GCTGCATAGTATTCTATGGTGTATATGTACCATATTTTCCTTATCCAGCCTATCACTGATGGGCATTTGG |
| | | ATTGGTTCCATGTCTTTGCAATTGTAAACATACATGTGCATGTATTTTTATAGTAGAATGATTTATATTC |
| | | CTTTGGTTATATACCCAGTAATGGGATTGCCTGGTCAAATTGTATTTCTGGTTCTAGATCCTTGAGGAAT |
| | | CACACTATCTTCCACAATGGTTGAACTAATTTACATTCCCACCAACAGTGTAAAAGCCTTCCTATTTCTC |
| | | AACAGCCTCACCAGCATCTATTGTTTCTTGACATTTTAATAATCACCATTCTGACTGGCATGAGATGATA |
| | | GATACCCATTTGTCAGATGGGTAGATTACAAAAATTTTCTCTCATTCTGCAGGTTGCCTGTTCACGCTAA |
| | | TGATAGTTTCTTTTGCTGTGCAGAAGCTCTTTAGCCTAATTAGATCCATTTTTCAATTTTGGCTTTTGTT |
| | | GCAATTGCTTTTGGTGTTTTAGTCATGAAGTCTTTGCCCATGCGTATGTCCTGAGTGGTATTGCCTAGGC |
| | | TTTCTTCTAGTTTTCATGATTTTAGATTTTACATTTAAGTCTTTAATCCAGCTTGAGTTAATTTTTGTAT |
| | | AAGGTGTAAGGAAGGGATCCAGTTTAAGTTTTCTACATATGGCTAGCCAGTTTTCCCAACACCATTTATT |
| | | AAATAGGGAATCCTTTCCCCATTGCTTGTGTGTGTCAGGTTTGGCAAAGATCAGGTGGTTGTAGATGTGT |
| | | GGTGCTATTTCTGAAGCCTCTGTTCTGTTCCATTGGTCTATGTGTCTGTTTACAAAACAGATTCTTAAGC |
| | | ATCAACCCAGATCGACTGGCTCAGAATTTCCAGGGAAGAGGCCTGGTTATCTGCATGTTTACAGACCTAT |
| | | TAGATTTGTGGGACCTGCAGTTCCCTTGTACAGTTAGTTACTCAATTAACATCTCCCTCCTCTCATGGTG |
| | | CCTCTACCTGCTAAGCCCTTATTCCCAGCCAGGCCCACCACCATCCACCCACTGCTGTTATAACATAAGC |
| | | AGGACCTGTGCGAGGGGGTGTGGACGGAGGAGAGAGGCTCTGTTGCTTCATTTGTGCAGCATGGAGTTCA |
| | | GTGGTTCTCACAATGTTTTTGCAAAGTATATAAAGAATACTCCTTGTCTACTTGACATTCGTATCGTGAC |
| | | ATAAATGTCTTGTTTTCCAGAAGGATTATTTTTTCCAAGCAGCTTGTTCCTAATGCAGCCCCAGGCACCA |
| | | AACAGATACTTAAAATATATTAATTGCTTAAATGGTTAAGAATTCAGTCTCTGGACCCACACTGCCTGGG |
| | | TTCAAATTCCTATTATCTGTGCCCAGTTTCCAAGTCTATAAAATAGGGATATTAATAGCACTTACCTAAT |
| | | AGGCTCGTTATGAGAATTAAATGAGCTAATTCATGCAAAGCACTGACATATAGTAAGCACTTAATAAATA |
| | | TTAGCTTTTTAACAAAATACAAGCCAAAAAACACTGCTTAGGAGAGGAAATGATGTTAGTGCCTCCTGTA |
| | | AATAGGCCCAGCCTCCAAGCTGGTGCTCCTCTAGGAATCACAACGCTGCAAATCACATCCTCCGGGGCCG |
| | | CCAGGACTTCACGAGGGCCTCTGAGCAGAGGGGTATGATGGGAGCAGAAGCCCAGCAGCTGTGATGATGT |
| | | GGTTTCTGATCTTCCTGCCCTTGGGGTGGGGGAGGAGGAAAGCAAGGGGCAATGAACAGAAAGGAGAAGA |
| | | TAGCGGGGAGGAAATGTGTGAGGAAGAAACACATCACTGTGGCTTGTCCTGGATTTTTCTGCTTCTGTTC |
| | | TCGTGTTTTGGGAAGTCTGGAGGAGACTTGAAAATCATTCATGTCCCCACCCTGAGGATGGCTTAGTAGC |
| | | AGAGAGGCCATGAAAACTCTTTGCTGATGGCTCTGAAAGCAAGGATGTTGCTTCACTGGGCTGCTGAAGG |
| | | CCTGCCTGGGGGTTCTGAGCAGAGAGTACAGGCCCCTCCCAGGAGGGCGGCCTAACCACCATGCTGGCAT |
| | | TTCTGTGGACCATGGTCTGCTGTCTCAGACCCCCTCCACAATAGGGTCTGCAATCTCATTCACCCCATAA |
| | | ATACATTCTGTCTTTCCTCTGATCCCCTCCCATTAGCAGGGGGAAATAAATGGAAGTCAGACGGCCCAGT |
| | | TAGAAGGCAGGCAGTGGAGTAGGAAAATAGATGATGGTGGTTTGGGGAGCCTCACATCACTCATGGGGAG |
| | | ACATTCATTCCCATGGGCCTTCCAATCACCCTTTTCTCCAAATCTAAGGACACAGGACAAATGGGTCCTC |
| | | ATACAGGCAAATATCTTAAACTGGTATGTGTATTCATTTATAGTTCTAATTTATATGTGTCTTTATTCAC |
| | | ATATATTTTGCTTCTGGAGAAAAGCTCAATTAGAAAAATTAATACATTATTCTTCTTATTGCCCTTCAGC |
| | | TAAAACAAGCATACACACCCCTCCCCTTTGGATTTTTTGTTTAGCAAAAGGTTAGGCCTGGCACAGATGA |
| | | AATACTATTCAGAGTTCACAGTGTATTTTCATTTCATAATATATTTGATTTTCAGGTCTTGAATTTCACA |
| | | TCAGGAAGCTGATATAGGAAGCTGAATTCAGCCAGATTTTAATACGAAAATACCTCTGATCAAGGCATAA |
| | | AATTGTACTTTAACCAGTAACCACTGTATTTCTCTAAGCTGTGAAAAAACATGCATTCATTAACTGCTTT |
| | | TTCCTCTGCTGTCAACACAGTCAATACATGTGCATAACTCCTTATTGTCTACATGGTGATTATCTTGCTG |
| | | ATGAATTCTCAAAGGCCAGAGATTTGGACTATTTTTTCTCTGTAACCTTGCATGTTCCTGGCCACATGCC |
| | | ACCACCACCCAAACAGAATGTACGCAGGGAATGTATTTTTCAGGATAACCTAAGAAAAAATAGGATTAAG |
| | | AAGATAAAGCTGCTGATCATGTAATGTACTTTAGACTCAGATATATAAATATTTGTGAATTATCTGTCCT |
| | | ATTTCTTTCTTCTATTAATTCATTGACTCTAGATGTGCATTGGAAGGCTAGGGAGAAATCAGGGGATCGT |
| | | GAGAAAGAGCACAGAAGTCTGCATCACACAAACAATATTATTTCAAGAGCCATGAACTAGATCCTAAGCA |
| | | ACTCATAGGCAATGACCTCATTTCATACCTCTAGTCTCTAAGAAACATATAACTGGCCTGAGGAAGGAAA |
| | | ATGTGGGCAAGGGGTAGACCGGGGTCATGGGTGGAGGTCCAAATAGTAATCAATGGAGCTCATAGGGTGG |
| | | ACTGATATTGAAGCTGCTATGAGCCAGCCACATGCTGGGCACTGTTACATGTCATCTCATGCAATACTCC |
| | | CAATTACCTGCCTAGTAAGCATAATTGTCATTTTATAGAATTAAAAACAGACTCAAAGAGGTTGACAGTC |
| | | TAATGTAACACAACAGCTAAATGGGGGATCTGGAATTATAATCCAGAGCTGCCTGGCTCTGATGAGAAAG |
| | | CTCTTTCTGCTGTCATATGCAGCCCACATTAATAGGGGGCTCAGAAAGTATTCTCTGGATAAATTATATA |
| | | ATGAATCCAATGAAGGAAGACATTATTTTATAATATGCAGCATAATAGGCACTATTATGATTGGATTTTC |
| | | CTGCTTGAAAGTAGCTAGATTAGAGTAGGAAACCAAAAAGATGTGAATTCATTCAGTCATTCATGCATTT |
| | | GCATGGATTGAGCTACCTACATTTGAATAAATGCTGTTAATCCCTGATTCCTTGGAAGCTCACATTGGAG |
| | | AGATAAGCATGTCATTAAATAATGCCATAATAGTGGTATCTCAGAGGACTAGCAGAACATAATTCAATCT |
| | | GACAGAGTAGAAACAGATTGTACAAATCCAATTCAAAACATCATAAATCCTCTAAGCACTGTCAATTCTT |
| | | CCTCCAAATTATCTCTGAAATTCCTCCTTCTTTCCCATTTATGGCCTCCATTTACAGAAGCGTGTACTGT |
| | | CTCTCTTAGCTGTTTGCCAGGCCGCCAGTCTCTTGCTGTTCAGCTCTCAACTGCTTCCAGCAAGATCTTT |
| | | CTAAAATCCCAGGCTTGCCAAGACTTAGCGCCCACAGCTCCACAGTGACTCCTCATTGCTGTTAAGGTAA |
| | | AGGCCTTCCCAGTCTAGCCCTTCATGCTTCTTCCATGTTCTATGGGACTGCCCCAGGCTTCCCACCTGGT |
| | | ACCACTGAGCCTTTCCATCCTTCCCCCACTCGACTGCCAGGTCAACACCCACACCCACGCTTCAGGACTC |
| | | AGGTCCTATGTTTCGGGCCTTCTTCTGTGCACCATTCCCTTCCCTGTAGCCCTTGATCATGATTTGTTTA |
| | | TACGCCTCCGCACCTTCATGGCCCTGAACCCCTCAAGGGCCGAAACTGCCTTACTTTTCTTTTTGACTTC |
| | | CCAACTTACCTTAGTGGAGCTGTAGTCACATAGAATAGACGCTCATAAATGCTTCTCTGGGCTGTAAAGG |
| | | TTGAATTTTCCAGCTAAGCAAGGAAGAAAGACAATTTCAGGCAGGAGGAAGGGCATAAGCAAAGTGCAGA |
| | | GATGTGAAGCTCAAGAGAAATGGATGGGCTGGGCAGAGGTGTGGCTGCAGCATCAGGGGAGAAGAAGTAG |
| | | TGCCTGGAGTCAGCAGGCACGGCTTGCAAAAGCTTCACCTATAGGTGAAAGGACACCATCTCTTGCACCA |
| | | ATAGGCTCTGTGATTGGAGGCAACTTTGCTGTTTTACTGCCAGAAAACTGAGGATGATAACCCAAACTGC |
| | | AGTTCAAGTGGCATTCACTGGTGTGGCTGAAATGGGTGTTTGTGGCCAGAATGTGGTCTGATTGGTCAGT |
| | | GCCCAGCTCTGTTGATTAGCAGATGTTTTGAATATAGTAGCATCCATGTGCCCAAGTTGTTGGGATGATT |
| | | CAACAAGAAACTTTAAGAGCTCAAGTGCCCTGCAGTTGTCAGCCAGGTGATTCTCTTCCTTTGGACCCAG |
| | | TTAGACGCAGGCATTACCTCGTGGCTTTGCCCCAGTGTGAATCTTTGTCCTCCAACTTGATCTTTTTATT |
| | | TGTTTCATTATTGTATTTAAGTTGTTTATTTTAGAGACAGACATTTTTTAACAGCTGTGCATTTCCTGTC |
| | | CCTTTGTTTTCCAGTCGTCATGTGTTTCCTTACTCTCTGTGGGTGAACGTTTCAGATGTCTGTTTGCGGT |
| | | GCCCAGCGTGCAAGATAAAATTTATTGCAGTGCCTTCGGCCTCTAACTCACCATTCCAACCAATTCAGAT |
| | | AGCCCAAGGCTGTTTTATCCAGTGGATTTTTCCATGTAGTGGGAAATAAATCTTGAATGTTACTGTTTAG |
| | | ATTAGCCAGGAAACTCATTCTGGGATGTTTGCCCACATCCATTGGCATTTCTCAAAAGGAACCCCAGGTG |
| | | TCTACCTTGACACCAGCAGGGCCACTTGAGCCCTCCGCTGGCATTCATCGCCCGCTTTGTTCTCAGCCTG |
| | | AGTTTAGGAGTTACAGATGTGAGAGGCGGGATTATACAGCCAACATCTCTAAGCGGGCAGTGGCTCCCTT |
| | | ACCCTCGAAGACCTCACTCCTAGCACGTCCTGGATGTATTCGTCAAAATATGTCCTCTTATGCCACGTCA |
| | | GCACAGGGTTGCTCCCCACTTTGATCATCAAGTTTAAACAAAAGGAAAGATTTTCTTTCTTTCTCTGCCT |
| | | CTACTGGACATCATTTCCCACCTAACAGATAATTTAATGTATCTGTTACTGAATGTGTTTGAATTACAGA |
| | | CAGAGAGGTCACAGTTAAAGAAGGAAGCCTGCTGCTACTGCAGCTTGTCCTCCCAAGGAGGTGTTTGATT |
| | | TAGCTGTGTAAACAAATGACTGCATTCTCCAGAGGTCCTGAACACAGCTGCCTGCGCTGGAGAGGGCTCA |
| | | AACCTCTTCCGCCAGGGTGAACTCTGCTTCCTGGTGAGTGCCAGCAAAACAACCAACAAAGAGCTGTAGG |
| | | ACTTGTGTGGACTTCAAATGGTGGTGGTCCTGCCACTTGGGCTCAGCCACAGCAGTTAGGAAACTAAAGG |
| | | GGAGGAGGAAAGCCCTTTCCTTGCTTTATTGTCATTGGCTGTCATAGGGCATTACAATGGTTCTCTTTGA |
| | | GATTCTGAGCTCCGGCTATAACATTTGCCCAGAATCTGCCTCTGAGGCCTTAAGACACTGTGTTTTTATT |
| | | CAGCAAAGATGCCCTTTGACTCCTTTTCCCACTAGTGGTGCTAGGTTTGAGCACCTTACACTGGCCCCTT |
| | | ACAATAGCCAGTTCTTGTCTACCTACATTCTTCCCTAACATTCATGATTGCATAGTTACTCTTAGTGTAG |
| | | AAGCAGACAGCTTTTACACATAGACTCCATGGCCGTAGCCTCATAGAACCTACTATATTCTAACTTGCAA |
| | | GCTAATCAGACCAAATATATCAAAATCAAAAACCTCTGCTGAGAGTTTATTCATTCATCTCTGTCTCCCA |
| | | AACGTACTTATGTACATACGTGCACTAATATACATGTCCATTAGCCAAGATTTTGATTTCAGGGATCAAA |
| | | GCAAGTACCAATAGGGAATGAGGTCACTTGCTGCATGGCAGGTGGCTTCCCCATGAGAATGCAAGGCCAC |
| | | CTCATGACTCATACTTCAGAGGGTGACCCAGGAACTTCTGATTCATGTCCAAAGCAGCTTCTACAATTGC |
| | | TCTACCTTGATCTAGGGAAGATGTGGGGAGGATGACATTCGGGATTAGCTTTATAAGGCCTTCCTGTGGG |
| | | CAGAGTTGTCTGACTTTCACCTAGTGATCAACAAGCAGCTAGCAAGCATCAGTGTGTGAGGCCCCACGCC |
| | | CTCTCAGCTCCCCTACTGCCCACCTGGGACATGGGCTTTGGCATCTGTCCATAGCATTGTTCTAACCAAA |
| | | TGAGGTGTTATGGATCAGCTCAGGATGGGATATGTTCCCAGACATATTATTTAAAGAAAATAGCTCCCTT |
| | | CCTCCCCTGATAAACAGCTGCCATGGCTAAAAGGTAACCTGGCTGGGGCTTAAAAGTCTGTTGACTTTCA |
| | | AGATATTTTGCAAAAACAGTCATAAAAATGGTATTTATCAGATCCTAACTATTTGTGAGACGGTTTGGTA |
| | | TACCATAGTGGTTAAAAACACAGGCTCTTTCCAGAGGAGGTTTACTTTGCTTAGTCGTGTCTCCTAAGTG |
| | | AACTTGGACCTCATAAGGTTGTTGTGAGAATGAAATGGGTGAATATGAGTAAAGTCCTTGGACCAGTTTT |
| | | GGCCGTATAGTAAGCCTTCAGCAAGCATCTGCTTTTATTCCTACAGGGAGGCAATTGTAAGCCCTTCACA |
| | | AACAGCGTCTAATGTGATCCTTAGAACAAACCTATGAGATAGGGCATATCTCAATTTTGTAGGTAGGGAA |
| | | ACAGAAGCCACACAATTAGGAAATGGCAACAGATCTGTTAGACTCTTAAACACTATGCTACACCAATTTG |
| | | CAAGGCAAGGAAGACAAAGCACCTTTGAAAATGGGTCAGATGTTTTAGGGTAAATGAACGTTTGAGAATC |
| | | TTTTAAGTTTTTTTTCCCCCAGAGATTATCAAGGTATCATTGTAGGGGGATGCATCAGGAAACATGACTA |
| | | TGAATCAGCTGCCTGATAAACCAGCCAGGATGGAGCCCACGTCATCACAGCAGTCAGCAATGCCACTGAA |
| | | AAACATCAGCTGCTTATTCCCGTATAGATTTCCCCTTAAGACATGAAAAGGGAGTTCAAAGAGAATGGGC |
| | | CAGATATCTCTGAGAGTCATATTACTAAAATATATTTATTTTTACTAGCTTTTTTGTTTTAAGAGGTATA |
| | | CTGTCATTAGCACTGTAGCAAAAATTCACGTTTTATTAATTTCTCCTAGTTTATCATGTGATTCTAGGGT |
| | | AGGATGCAGAGTTATATTCAAAATACACAAATCAACTCAACTCAGTAAACATATATCGAGGCCCTATCAT |
| | | GACAAAATGCTATTCTAGAGACCACGGCGAACAAGCCACGGCCCCAGCCTCAAAGAATGTACTATCTTTG |
| | | GAACTGTGCTGGCCAATACAGTAACCAGCAGCCACGCAGGGCTATTTAAATTTAAATTAATTAAAAGTAA |
| | | AAACACAATGCCTCAGATGCATTAGCCACATTTTAAGTGTTCAATAGATATTTGTGGCTCCTGCCTGCCA |
| | | TATTGGACAGGGCAGATATAGAACAATTCCATCACTGCAGAAAGTTCTACTGAACAATGCTGCTCTGGAG |
| | | CAGAAGATCTTCTTGTTCAGGGATGTTACACCCCCGCTTGTGGCTAGAGTGTGGCTTATCCTCAGAGCAA |
| | | GGATAGGGGAACCATGGCACTCTGCAGGCTCAGCACTGAAGACACGGATGCAGGCTCTGCTTCTGACCTA |
| | | GATTGACCTTGGGCAAGGCCCTTTGCTCCTCTGATCCCAATTTCTTCACCAGCCAAGTAAGAACATCAGA |
| | | CCACAAGCCCTCTAGGGCTCTGTCCAAATGCCCCATGACTGAGTGAACTGGTAGAACATTCTATGTGTGT |
| | | GTCACAACATGAAGAGCAAAGACTTTCATCTCCCCAAATAATTTTGTTTTTCGTTTTAGGAATTAAATTT |
| | | CAGATTCACTCTAATTGCCAATACTAAAATTCTCTATATGCAGTTCTAAACTTGACAAACCAATAAAAAA |
| | | AGATTATTTGACTACTTATCTTTGTACAACATTGAGGTCTCCCTAAAGCAAATTTAAATGCATATTTTAA |
| | | AAATGTATTCTAGCAGTTCAGTTCAGAAGCCCCCTGGCCCAAGCATCACACTGTCAATCCTTTGTCCTCA |
| | | AGCAGCATGGTTGGGTGGGTTAAGTACTGACAAACACTGGGTGTCAGGCCCATGGTCAGGGACTGTGCTA |
| | | ACAGTCTACATATTAGATGCCACCTACCCCCACCCTCAACAGACCCAAACTATTTATCCAATAGCAAACC |
| | | TTGCATTATTTCTGTCCAGAAGAAACAAACATTTATTGACAACTTTTGGTGTGTGACCTGTTTAAGTCCT |
| | | ACATCTCATTTAAGGACTGGTCAATGTTAGGCTAGGCAATGCCTGTTTGTGAGAGAATCACTGCCTAAAG |
| | | AAAATTCTCCATTTCCCTTAGCTCTATGGTGGGTGACTACACATACTGGTATTTCTTAAAGAAATACCAA |
| | | TTCCATTTCCTTTTAACATAATTATTAATATCTCATTAGCATGGTGTCACTGAAGCCTGGGCCCAAAGAA |
| | | ATACCAATTCCATATCATTTTAAGATCATTATTAATATCTCATCAGCGTGGTGTCACTTAAGCCTGGGCC |
| | | CTTTAGAATTTTTCATGTACCTGTGTTCCTCTGCCCATATCAGCTGGAACACTAATAGTTTTCTTCCTTT |
| | | TTATCTAGAAGACTGAGAACATTACATGGGACCTGCCCCCAGGGCATGGAGGCTGAGGTGGGACAGTTTA |
| | | GTTCAGGAGGCCCAAGAAGTGTTGGGTGTGCAGCCCCTTGTTCAAACACAGCCTCTGAATCGCCAGAGGC |
| | | TTCCGGTGCATACTCTGAGGCGCAGGTGGGACTCGGGAGTGAGAGGTTTCGGCGAATGAATTGGGATTGC |
| | | CTACTTCTTCCCAGTGCAGTGGAGCTTGGTTCTGTGGTCAGGTCCTTACGCCCTGTCTGCCTTTCTCGTT |
| | | TCTTTATTTCTCGGGTAGTAGTTGTGGAATCAAATGACCTGGGGTTTGATACCTACTCTACCACGCCTCT |
| | | GGGGGAGTCACTCAGACTCGTTGAACCTAAGTTCCGGGGCTGCCAAGTGAGGATAAGTAGTAATTGCTGA |
| | | TCCACCTACTTGACAAGATAGTAGTGAGGGCCCTGAGCGCCAGGCTGTGGATCCAGCCTTTCCCACGGTT |
| | | CCTGGTGTGGCAGGAAGAACTCTAGGCCTGAAGGTGAAATTGGGGAGGGAGTCCCAGCTCTGCCACTGTC |
| | | TCTCTGGGTGACCTCAGGCAGGTCTCCTCAAAAAAATAAGATACTTTATAAAGCTCAGTTTCCTCTTCAG |
| | | TAAAATGAGGATTCCAGGTAACTCACAGATAGTTTGTGGGGATGAATCTGTTCCTTAAAGCCTGCAGTAC |
| | | ATCAATAACCCAGTCTTCCTGCTTGCTTTCCCCCCTCTCCACTACCAGTGATCATAGTCTGATCCCATAG |
| | | GTGATATCCCAGCTCAAAACCCTACATTAGCTTCTGTGGCTGTTTAAGGCCTGCCCAGAACTCCCCTGGT |
| | | CTTAGCACTGAAAGCACGTGTCCGGGGAAGCCCTGCATTGGTCGTTCATACTACTGAGTCCCGCAGGGCA |
| | | AACCGTCCGGTCCCACCCTCCTTTCTAGTGCTGCTGTCACACTCACCTCCCTTCACCCTACACTCCCTTC |
| | | TGTGCCTTGCAATTACCTAGGGAGTTTTTTACAAGATATGGATGCCCTGGCCCTGCCACTAGAGATTCTG |
| | | ATTTAATTGCTTGGGGTAGGGCCTGGCATAGGTATCTTTTAAAGCTCCGCAGTGGTTCTAAAGCACAGCC |
| | | ACAGATGGGAACCACTGATCTATTCTTGTAGGTCCCCAGATACCTCATGTGCTGTTCCCTGTGCCTGAGC |
| | | TGACCTTTCCCCCACTTTCCTCTCCTCGGCTAATTCCTGCTTATCCTCCTACTCAGGAGGCTCTTCCTCC |
| | | AGGCAGCCTTCCCTGATCCCTCCAGGAAGACTTAGCTGCGTCCCTCCGCTGGGCTTCCCCAATACACTGG |
| | | GCTTGCTTTCATTAGAACCTGATCCTTCCACATTATGGTTGTTGGTTTGCTCCAATCCTCTCCCTCATTA |
| | | GCTCTCAACTTTCTTTCAGGAAGAGATGTTTATCTTTCCTTCTTGTATTCCTAGAGTCGACCAGGCTCTG |
| | | GCACATTGCAGATTCTCAGTATGCATTCAGGGAACAACTTAATCAAGACAAGACCATCTGACTTCTTGTG |
| | | AGTTACATGCTAAGAAAGAAATGTCGACACCAATAGCCCTCACAATGATAGGAACAGGAGGTTAAAGAAA |
| | | AGGAAATAGATGCAAATAGCAATATAAGTGCTTTAACAAATCTATACAGGAGGACAACCATCATATTCAA |
| | | ATTTTCAAACATTCTTAGTTCTGCTCTTTTGTGGGTAATGGTTTTTTTTTTTCCTCTTCCAGGAGAAGAA |
| | | AAGAGGCATATTATAGAAATTCCTCCTCCCCCAGCATTACTTGTCACAGAATTGTAATTGGAAGTGATTT |
| | | CCCTGACTAAGTTATTTTGGCTGTCTGTTATTTTCTCTCTTCCTCCTTGCTCTTCCCTCAGCTGGCCATC |
| | | CTGTGTGTTTGGAGAGAGCCAGAAAGGTTCAAGGCTAGGAATGTTTCTCTCTCTCTTTAAAGCTCTTTAA |
| | | TCGTCAGGCTTTCTGATCTTCAAAGCAGGCTGTAGCCAGTGTGACCCCACTCCCTCGCCTCCCCATGCTG |
| | | GAGAGTAAAAGCCTGGAGTATTTTTGTCATTTTGAAGACTTGCATATTTGGACAGCCTTGGACATCTGGA |
| | | AAGTGTGGTCCTCACTAGCTCTGCAGGGATAAGAGCACGTCAGCACTTCCAAGCTCTCTGGCGCCCCTAC |
| | | ATCTGGACACGTTGAAAAATTAACACCAGACTCTGGAGTTAAGCAAACATTAAGTTTATAGGCCTCCTTG |
| | | CATTTGACCATTTCCTGGGACAGCAGCCCTTATCCTGTGACTTTCTGTGTGTAGAGTTGAGTCTTTGCAG |
| | | TTGGTCCTCCTCACACTCTCTCAACTTTGTGACTCTCTGCAGTGCTTGGTCCTGGATAAGTTTGAAAGCT |
| | | ACAATGATGAAACTCAGCTCACCCAACGTGCCCTCTCTCTACTGGAGGAAAACATGTTCTGGGCCGGAGT |
| | | GGTATTCCCTGACATGTATCCCTGGACCAGCTCTCTACCACCCCACGTGAAGTATAAGATCCGAATGGAC |
| | | ATAGACGTGGTGGAGAAAACCAATAAGATTAAAGACAGGTGATGTTTCAGGAAGGGCTCGCTGCATTTCT |
| | | CCAAAGTCAGTGGGAAATTACATTTGGTAGAGAGAAAGGGATTGAGACTGGACTCATAAATCAATAAAAT |
| | | TAAGTTAAATAAGAAAAAATAAGATATTTTATAAAGCTCAACAAAGAGTCCTTGAATGAAAGCAATTACA |
| | | GAGTCACATTGTGGCTAATATTCAAAACTGAGATTTAAACTGAGGACTAGGAAATAGAATTGGATCCTTT |
| | | TGAAGCGTTTAGGAGAAAGATTTTAAGAGAATGAGTTCCGAGTCACCCTGTGGTCGGGAGGTGTGAGTGA |
| | | GCTATCCAAGCCCGTTCCCATCCTTTGTCCCTCTGTGTCTTCTCAGGTATTGGGATTCTGGTCCCAGAGC |
| | | TGATCCCGTGGAAGATTTCCGGTACATCTGGGGCGGGTTTGCCTATCTGCAGGACATGGTTGAACAGGGG |
| | | ATCACAAGGAGCCAGGTGCAGGCGGAGGCTCCAGTTGGAATCTACCTCCAGCAGATGCCCTACCCCTGCT |
| | | TCGTGGACGATTCGTGAGTCTGAAGTTCGCGATCCTCCTCCATGACACGCTAATGGGGGTGCTGGAGTGG |
| | | GCTGGGGTGGGCTGGGGGTGCCCTCAAGGCTTCCATGTCTTTAGAGAGAGCCCCAGGGACCAGAGCCAAA |
| | | TTGGAGAGCATGGAGCTCTGACTGAGGAACCTGCTTCTCCCAAGCTCCAGGCAGGCACAGATGAGTCAGT |
| | | GCAGTGGTGGGAAAGGGAAAAGAGTTGATGTTGTAGCTGGAAAAGGGAAGGGGAAAATTAAAGCAAGGAA |
| | | AGTGAGGCTGGGGGAGGGGACAAATTCCCCACTATGTAGTATGTTTGGTATGTGGAAGGGTTCTGGTCAG |
| | | AATGTTTGCCCAATGATTGCCACATCAGCATTCATTTTGGACTCTGTATGGCCAGTAGGTCTGGTTCCTG |
| | | GGAGCCCTGGAATAATGCAGCCCCTTCCCTAACTAACATTTCCATGATGTATGCTCAATGACAAGGCAGA |
| | | GGAATGTGTTGGATGAGCTCAGGACCTGCCTCCCTGGACACTCCCATCCCAGGCCTGTATATCTGTTGAC |
| | | CAGGAATAAGCCAAGCAAGCAGCCTACTGTTTGACTGAATATGGATTTGGGGGGTGGTAGAGAAAGGGCC |
| | | GGGGTGGAGGGTTGGGAGGCTCATTTGTCATTATAGATGGGGTCAGACACACTACCAAAACAGCAGCAGA |
| | | GATCTACAATTGAGTTCACCTAAAACTCAGTGTGGACACAGGAAACCCTCTTTTAATAACTGTCCAATGG |
| | | GTTTTCCAGCCTCAGCTCTACAGAAAACTTGAGATAACAGTGGCCAGTCTGCAGTTAGTTTGGGTTCGGA |
| | | CAATAGGCAGAGCTGGGAAATGGAGCCAGGGGCGAAAGCCCAGGTCCACTTTAGGATCAGGACGGGAGTG |
| | | GCTGGTGGGGAAGTGAGGTGGGTGTGGGGAGGCAATAGGGAGCTGGGTCATTTGGTATGGGAGAGTCCTC |
| | | TGGTGGCTAGTCCCAGAAGTGCATGCTTTACGAACATATGCTTCTCTCCCTAGGGCCACCTTGAGTGAAA |
| | | CCCTCCCATGCTGGAATTGGGCCCTTTCAGTGACAACACACAACAGTTTTCAATAGATAATAATCCCAAG |
| | | GGCTTTACTAGCACATGAAACACAGGGAAAACGTGTAAAGTTCACAAGAAAGTCGTTCCAGTGTATCAAA |
| | | TCTATCCTGTTTGCCAGGTGGATATACCAGGGTCTCCTCCACCTGTGCATGGCTGGTGGTGGGTCCAGTG |
| | | GCTGTTGGATAACTGATGTATTGATGGATCATTCGCCTTCTGAAAGTGCCAAACTGATTAGTTATTTTGT |
| | | GTGTCTTTTTGTGTAACTAGGGTTTGACCTTCCAGGGCAGACTGTGCTGGGGCGGCTGACCCCTTGGGGA |
| | | GCCAAGTTATTGCTCTTACCACCACCACTTGCCCTTGTCAGTCCTCCACCCTCTTGGGTTTCAGTGTCAG |
| | | CATGTAGCTGTCTACTCAGATCCCATCCACATCATCAAGTCTGCAGTTTTTTCCTTGCAAGGCCTTACAG |
| | | GGAAGATCTTTGACATAGAGGATATAATTTTATTGACACATTTTACTTGCAGAGCATTCACCCGGGCTAA |
| | | CCAGAAAGCCAGCACTCTGCTATAAACAAAAAATAATGCTTCAGGGCTAACATGGAATGTGTTAAAAGAT |
| | | TCCAGCCCATTAAATGTCCAGGGGAGGTTTTCCTGTTTTCCTTTCCCTCCATCTGGGCTTTGTTCTCAAC |
| | | ACATTCATTCAACAAACATTTATTCTGCCTCTACCAGGTACAGAGCACTCTACTATTCTGCTTCTCTCCT |
| | | TTTGCTTTAGTTTCATGATCATCCTGAACCGCTGTTTCCCTATCTTCATGGTGCTGGCATGGATCTACTC |
| | | TGTCTCCATGACTGTGAAGAGCATCGTCTTGGAGAAGGAGTTGCGACTGAAGGAGACCTTGAAAAATCAG |
| | | GGTGTCTCCAATGCAGTGATTTGGTGTACCTGGTTCCTGGACAGCTTCTCCATCATGTCGATGAGCATCT |
| | | TCCTCCTGACGATATTCATCATGGTAAGCCAAATGGAGAAGGCCCAGAAAATCTTGAATACTTTGGTTCC |
| | | TTTCCCCTTTCCTCCTGTTCATGTGCCTGGATTAGTCATGTGGCCACCAAGGAGAGCGTGACATCTAGCT |
| | | TCCCAGCCCTTCCTTTTAGCCAACGTGGGAGACACTCAAAGAGACGAAATCTCCTGAAGGAGCCACTGTA |
| | | TCACAGCATCCTCCCATCTCCCACTTCCTGCCCAGGGGTCCATGGTCCACACAGACTTCCCAGTCCCATT |
| | | CCGTGACCATCTGGAGAAGCTGCTATTAGCAGAGCCCTGCACAGGGTGATAGTGTAATTAAAGTGGTCTT |
| | | CTCTTTCCAAACACAGAAAAAATCAGTTCAGGGAGTGTTTTCCTGGGCTTACAATTTTAACTACTGGCTA |
| | | GAGTTGAAATGGGGAAAGCCTTTTGCCTTTTCAGTAGCAGTAGGGGAGGAGATCTGGATTATTTACTTAT |
| | | CATCATCATGGTCACCTCCTACATGGCTTCACCAAAAAACATTCTGCTGCCTGAAAAAGCTCCAACACCT |
| | | CTCTCTCTTTTAAAGGATGGAATTTGGAGTCCATCCTTCCTCAGTGATAAGGAGTTTTTATAGCCACAGG |
| | | CAGCATCTATTGGTCTGTCCTCTGCAAACTTGCAACTCCTCTGAGAGCTAGACTTGGAAATGAAACATTA |
| | | TTTTGCAATGCGCTGCTATCCTTCATTTTTAGCTCCTCCACCGTAGATGATAGTTTGTACTTGTTAAATG |
| | | ATAAGGATATAAATTTAGGTCATTTTTTATATTTTATTGGGTGGAATTTGGTATAATTTTTAGACTTCAG |
| | | GCTTTACAGGCTCCTGAGATGGACTGATTGAGCTTGTTCTACTTCTTCCCCATCATGATAGGAAGTGCTG |
| | | TACCACACTAGGCAGTGTGTGTAGTGACCACAGACTGGCTGAGTGTCTCCCATCCCATGCTGGCCCATAT |
| | | CTGGTACCCACCTGATCCACAAATGTTCCATCAGATCCTGTTCAAACAACACATCTCCAGTTAAGCCAAA |
| | | TCTTGCCCTTTCTCCTTACGGTAAAATGTACTAAATCTGAAGGTTTTGTCTTTTTAATGTTGCTCCATGA |
| | | TCCAGTGATCTGTGGCCTTGGTTATGCTCTGTGCTAGAGTCCTAACAAGACAAATGCTAAGGTAGAGGTC |
| | | ATTCTGCTCAAACAACCTGACCCCACCTGGATGTGGGCTTACATTTGCAAAGGGCACCAAAGTTCTAAGA |
| | | GATGAGGGGAGGAGCTGAGCCCCTTGTCCTTATCTAGGTTTCCCTTGTTCTTTCCCATCCCTCAGTCTGC |
| | | TTCTTTTCCCAGTACCAACATGTTTGTGTCCTCAGAATTAAAGGAGTAAAAATGTGTAAACATCTGACTA |
| | | GCAACAGCCATGAGATTTTGCCTGGCTTGTTGATAAGCAGCATTGAGATCTGCCCTCCTAAGAATGGGCC |
| | | ATTAGGTCTTCAAAGCTTTTACGATGTGAGGTAAAGAATGTTCACCAGGAGTTTCATGCACAAAAGGGTT |
| | | TCTCTTTGTGGGAACTAGAACATTGTTCCAGTGATGACGGAAACAGGGCTTTCCATACCAAAACAGGGTT |
| | | TTCCTTTGAATGACTCTCCCACCTTTCCCTTGTCTCTTCCTCCCCACCTCAACAACACAGGAAAGAAGCT |
| | | GGAAGCAGGGACAATGGGAAGGTCCCTTTGTTACTCGAGCTATTAGAAACAAAAAGAAAAGTGGCCATCT |
| | | GAGGAAGCCACAGCTGGTGAAACTGTAGGGTCACAGAGTGAATTACACCTCTGGCTTAAGTCAGTGAAAA |
| | | GTCCTAGAAGTTTGTGGTCCTAGAAGTCCTAAAAGTTTATGGGACTTTGTTTTGAGCAAGGATAAGAAAT |
| | | TGATTTCAGGCTGGGCGTGGTGGCTCACGCCTGTAACCCTAATACTTTGGGAGACAGAGGCAGGTGGATC |
| | | ACTTCAGGTCAGGAGTTCCAGAGCAGTCTGGCCAACATGGCGAAACCCTGCCTCTCCTAAAAATACAAAA |
| | | ATTAGCCAGGTGCGGTGGCACATGCCTGTAGTCCCGGCTACTCAGGAGACTGAGCAAGGAGAATCCCTTG |
| | | AACCCAGGAGGTGGAGGTCTCAGTGAGCTGATATCATATCACTGCACTCTAGCCTGGGCAACAGAGCAAG |
| | | ACTCTGTCTAAAAAAATAAATAAATAAAAAAGAAATTGATTTCATTCTTCTGAGAACTGCAACAACTACC |
| | | TTAAAGTGATTCCATCCAAAACCCACATGTTCAGCCATGGACTTGCTTTTATGGAGCTGCGTGTGGGTGA |
| | | CACACAAAATCAGGAGCTCTGAGTCCTAATTTAGACTTTTATTTAGATTTCCTCAAATTTGGGTTCCAGT |
| | | TAAGCGTGGGTCTCTTCTGTGCCCCGCTCCCCTTTGCCATTTGTTTTATCTGTTCTTCAGTCTGTTCTGT |
| | | CAGTACCCACAGGCAGGAGAGCAGAAAGGAGAAATGGCAGCCACAGCAGACAAATGGCACATTCGTTCCA |
| | | CTCAGCTCTCGCATGCCCATCACAGATACAGCTCATTGGTCTCTTTTCTATGAGAGGAAGCCAGAGCTCC |
| | | AGGGAACTACTGCCAACTGATCAGAACTCATTTAGGACATGGACCTATTTGTTCCTTTATGTTCCTGGGA |
| | | AGAGCACAGGATGAATTCTATGTACTCATTTACGTGTTCAGAGAGTAAAGTGCCTCATAGGATGCCTCCA |
| | | GCAAAAGATAACCAAGAAGGTCTAATACCTTTGACAATCTCAGTTTATCCTATAGTGTAATTGGATAGCA |
| | | GTTCCCCTAGCAAAAGTTGCTAGTTTGGTCCTATTTTCTACATAGCCAAAGTGATTGATTCATTGGTTAA |
| | | TGTGAAAGTTACTGAGTACTGCCAGCAGGTTCTAGGAAATATATTTGTGTGATATTCATGGATGGGGAGG |
| | | ATCAATCCACTTCCAAGTGATTTGGATTAATTACTGGTATTTTCACCTGTGTGGGTAGCAAACCTCAGAA |
| | | AATCAAGTATAGATGACGGCATAGGACAGGCCAGGCCCCAGGCAAAATGTTGAAGCTCCTCTGGAGTTCC |
| | | CTCCCATCTCCCTCTTTTGTTTTCCATATACCTGGTTTATCCAGGGCCCTGGAGATGCTCCAAGACCCCC |
| | | TACCCAGGTCTTCCTCCCTTGTCCCAGCTATATTTCTCCATATTACCACTCTTCTCACCGAGGATTTGCT |
| | | TACTTAACACATAATAAATACTATTAAAAGAGAAACTTAGGCACATTAAAATGTTAGAGTTGATTCCAGC |
| | | AAACAGTGATTCACAGGAGGCTCCAGATCACAAGTGGTTCAGGGCCCCACTGAGGGGTAGGGAAGCAAGA |
| | | CAAAGAAAAACAAAGCAAATATTTGATTGGTTCAAGTGGAAAGTCCCTGATTACAGGTTAGTGGGCAGTT |
| | | TGTGATTAGTTAAGTTTCTCTAAGTTGGGTTTTGGTTTGCTGATGTAGGAACACAGAATGCTGGGGCCGT |
| | | TTCAACCTAATGGTCTCCCAATTAATTTTTTTAACATTACTGATGACTGTTAGGAGTCTAATGTGCTACT |
| | | CCTCCCAGGGAAAATGGCATTCCTAGGATTAAAGGAACTCAGCACATGGAGTGTGCGTAGAAATTTAGAC |
| | | ACTAACTGCAGGCTGGTGGGAGAGAGCCCTTTAGGGCAGAATGAGAAGGCGTCCGGCCAAGGGCAGGAGT |
| | | TACTGACGCATGGCCTCTTGGTTTCAGCATGGAAGAATCCTACATTACAGCGACCCATTCATCCTCTTCC |
| | | TGTTCTTGTTGGCTTTCTCCACTGCCACCATCATGCTGTGCTTTCTGCTCAGCACCTTCTTCTCCAAGGC |
| | | CAGTCTGGCAGCAGCCTGTAGTGGTGTCATCTATTTCACCCTCTACCTGCCACACATCCTGTGCTTCGCC |
| | | TGGCAGGACCGCATGACCGCTGAGCTGAAGAAGGCTGTGGTGAGGCCCTTGGGCTGGCCCCTGTCCTACA |
| | | ACACGTTTCCTTGGAAGGGTCCGTAGCAGTCCTGGAGGCCCAGCCTGCCCTCTGAGGGGGTCCACTTTGC |
| | | CTTTGACCTAAGGTTAAAAAGTTCACGTGAGGCTAAAATGTACAGGGGCAAAAGTGGGAGCAGTCCTCAC |
| | | CCCGAGCGATGCAACAGTGACTCCTCACCACGCCTGCTTGATTCATCTGCCCTGGAAAGTCATTAAAAAA |
| | | CCAGTTCAACTCATGGGTCCCTTTATTTACTCACAAGAGAGAGCCAGCAGCCCATTTCACTAGTTTTCCT |
| | | TTCCTACTCTTTGAGAAGAATCAGAAGGGAGGGAGCTTGCCACTTTACTATCTGTCTAAAGAGATGTTTC |
| | | CATTAATTAAAGGTTTTTGTTTTGCTTCAAAAAAACTTGAATTGGAGTATTTCCACAAGTATCTTTAACA |
| | | TGCTCTACCAATGTTTGCAGAAAGAAGTGCAGAAATGAGACTGTCCACAGAGTCAGGCTCGCTGGCCAGG |
| | | AGAGGACTCCCGAAGCTGACTTCTGATGGCCTGAGAAACTTCCTAGTTCACAATTCCCAGACCCAGACAA |
| | | AGAGCACTGTCTTTTCTCTAATTGTTTTCAAATGGGCCATTTCCACCCTCTAATCAGCCTCTGGCCCTGG |
| | | AGGGTGCAGTTCCCCTTGTCCTCCGGAGTCTCCCTGTCTCTGTGCTGTAGAGTCAAGAAGGGACAACCAC |
| | | CTGCCCTCACTGGGAAAAGACAGAAAGTCTGACTTGTTCTCACGACTCACACTTATTAGGCTCCAGAGGT |
| | | GTCAGGGCATCTGCCTTTCATTTCTTAGGTTAAATAAGAAATCAATTGCTGCCATTTGTAGTACCCAATT |
| | | TTCTAAAATGATCACAATGGATAAGTGGCAAGAAATCCTTATGACTCATCTGTGGGCAGAGTTGGGCTAT |
| | | TTTGGTAATCCTTGAGTAGGCAGATGGAATTTGAGGCCATCTTCTTGGGTACATAGATCACTAGGAAGCT |
| | | ATAGGTCTAGCAACTGTGGATTAGGGCTGGGCTGAGAATTGTTTCATGTTTTTTGTGACTGTATAGCTAG |
| | | AGACTCTCTTGTTTGCAGAGAGACACTCTGAACTCCCCCTGGCCGTCAAGGGAAAGACTGCCTTCACCCT |
| | | CCTGAGCTGACCTTACACTGAGAGACAATGGGGACCCTCTTTTGGCCCTCCCCTCTACCTCGAGGGCATC |
| | | TGGGTGCTGTTGCATTGGATAAAAGGCACTGCTCTTTTTCTGTGCCCTCTCCGCCTCACTGCAGAGCTTA |
| | | CTGTCTCCGGTGGCATTTGGATTTGGCACTGAGTACCTGGTTCGCTTTGAAGAGCAAGGCCTGGGGCTGC |
| | | AGTGGAGCAACATCGGGAACAGTCCCACGGAAGGGGACGAATTCAGCTTCCTGCTGTCCATGCAGATGAT |
| | | GCTCCTTGATGCTGCTGTCTATGGCTTACTCGCTTGGTACCTTGATCAGGTGTTTCCAGGTAAGCATCCT |
| | | CCTCTATAGGGTAAAGGTAATTGAGTTCTTCAGATCCCCAGCCCTCTCCATTCATCTAGTTTAAATTTCA |
| | | TTTCTTCCAAGCTCTTTGTCAGAACCAGCATTTGAAGTTTAAATCTAGAAGTTAAAAATCCACCAGCAAA |
| | | TCCTACTGGCTCTACTTGAGAAACAAATCCAGAATCTGATCTCTTGTCACCACCTCCACCACAACCTTCC |
| | | CAATGCCAGTCTCTTCCTTCCACTACCACCTCCCATCAGTCCATTCTGCACACTGTATTCAGGGAGATCC |
| | | TTTCAGAATCAAGGTCATGTGGTGTCAGCCCTCTCTGTCAAATGCTTGCACTGGCTTTTCCTCTCTTTCA |
| | | GAGTAAAACCCAGTGTCTCAACCCTGGCCTCCAAGCTGCTTCATTATCCGGCCTCCAACTCTCTTCTTCA |
| | | TCTTACGATTTTCCCTACTCCTCCATGTTCCTCTGCTCCAGCCACGTCGGCCTCCTTACTGACTGTTTAA |
| | | TACACCGAGCGCATTTCCTCTTCAGGGCCTTTCCACCTGCTGTTCTCATGCCAGAAGCACATTTCTCTCC |
| | | CCACAACCTGCAACCCGCCCCTCATATCTGCAGGCTTGCTTCCTTACTTTGTTAAGGTCTCTGTTCAAAT |
| | | GTCCCATTATCACAGGGATCTTTCCAGACTGAAGAGATCTACATAACTATGGCTCTGTAAACAACATTCC |
| | | TCCAGGGTTCCTGTCCCCTTACCCTACTTTATTTTGGGGAACATTCTTCACCATCTGATACAATGATGTA |
| | | TCTTATGCATGTATTTACTGACTCTCTGCCCTTAGTAGAATATGAGCCCAGAGAGCATGCATGTGGTCTA |
| | | TTTTGTTAACTGTGACAGTCCCAGTGCCCAGAATAGTGCCTGACCTTTGGTGGGCACTGAATAAATATCT |
| | | AAGTAATCTGTAGCATGGAAAATCAGCTTCTGAAAATTGGCTGTTTGCACGGTCGTGTATTTGCTTGGTA |
| | | GAAAATCAAATTTTCCTTCAAATTAGCATTTTCTGGTAACTAGAGCTGCCCCATCTTCCTCTGAGTGGTC |
| | | TCCAAGTCAGCCAATAGCCTTGTGCTGTGGCAGCCATGCCTGGCTCTTGATGCTGTAGCCAAAAGCAGGC |
| | | AGGGGATGGTGAGGCTGGTCCAGTCCATGGGGAGGGACAAACTCACAGCTCTCAGATCATCTCAGGGCAG |
| | | CCTTTGTTGGCAGAAATAGGTAGGCAGCCACCCTGAATAGGAGGAAGGCTTCTAGACTGGGTCAGGAGGC |
| | | CTGGGTTTGCATCCTAGTGGCAAGCGTGCATTCATTTACTAGGGCTGCCATAACAAAATACCACTAACTG |
| | | GGCAGCTTAGACAACAGCCATTTATATCTCACAGCTCTGAAGGCTGGAAGTCCAAAATCAAGGTGTTGGC |
| | | AGGGCCATGCTCCCTCTGAAACCTGTAGGTGCTTGGGCACTCCTTGACTTGTAGATGCTTCCTGCTGATC |
| | | CTTCGTCTGCACATGGCATTCTGCCTGTCTTACATGGCCATCTTATAAGGATACCAACTGGATTGGATTA |
| | | GGTGCCTACCTTGCTCCCATGTGACCTCATCTCAACTAATCACATCTGCAATGACCCTGTTCCTAAACAA |
| | | GGCCACATTATGAGGTACCTGGGGTTAGCACTCTGGTATCTTTTTTCTTGACAGCACTTCTGACACCAAA |
| | | TGTGTGTTTTGGTTTTTTGTTGTTGTTGTTTTGGCACCAACCAATTCTCCTATATTAATGGGTTGTCCAA |
| | | GAATTCAATTGAATTCTGACACTATCCAGAATTCACACAGACTCCACGGGTTCAGTCCCACAAGGCTTCC |
| | | CCGTCTTCAGATGCCAGCTGGAAATGTGGTGCCCAGGCTACCCACACTTTTGCCAAAATCCTGTACTTAC |
| | | AATCACAGCTTTAAAATGAAGGATGCAGCTCAGGAACTGCCACATGGAAGAGAAGCACAGTATGGGGTCG |
| | | GGGGAAGAGTTTCTATGCTCTCTCTAGACGCACCACTCTCCCAGCACCTCAAAGTGTTCAGCAACCCAAA |
| | | AGCTCTCCAAATCTTGTTGTTCGAGAGTTTTTATAACCCTATCTCCAGCTCCATACTCCCCCATTGGAGG |
| | | TTGAGGGTTGGGACTGAAAGTTCCATTCTTCACATGTGTGGTGTTTCTGGTGACCAGTCCCCAGAAACTG |
| | | CAGCTATCTTGGGGCTCTACCCTGAGTCACATCATTAGCATAAACTCAGATGTGGTAGAGGAAGGGGCTT |
| | | ATTATGAATAAAAAAAGACACTCCTTTCTGCCAGGAAATTCCAAGGGTTTTAGGAGATCTGTGCCCTGCA |
| | | CAGGAGCTGGGGACAAAGACCAAGTATATTTTGTGTTATGCCACAGACCCCAACATGTCTTTTTGGAGGG |
| | | AGACCAAATTCAACCCATGACAGTGACTTTGAACAAGACATTTGAACTTAGTCTGTTTTTTCTATCCTAC |
| | | TAGATTGTTGGAAACAGATATAATAGATGAAAATTAGTTGATTAAAATTGAAATTTGTGCATAATTCAAA |
| | | AGTTTTATTTTAGCCAAGCTAAAGCTTTCATTTATTCAACAGCTATTTACTGAGCAGCACCTGTGCATGA |
| | | GGCTCAGCAGGGCCAGGTTCTGGGAACAGAGCGGTGGAGATAAAGATCCAGACCTGCCCCGAGGAATAGA |
| | | CAGTCCAGTGGCAGCAAAGGCCATGAAACATACGGCAACTCTTAAAAAAAGCCGAGACCATGATTTTACA |
| | | AAATCAACATTTTGTAGGGAGCAGAACTTTCAAAGAGAACTGGACTAGAAATTTGGGAGTCTTTTTCTTG |
| | | GAACCCTGGTAGATCCAGTAGAATGAGGGATGGGGGTGTAGGGTTAAAAACACTGACATTAGAACTGGAT |
| | | TACCTGTGTTGGAATTCCTACATTTCTGTTTCACTATCTGTGACGGGGGGCAGATGGCTGAATCTCAGTG |
| | | TGCCTCTGTTTCCTTTCTCACAAGAATAATATTACTACCTATCTCCTGGGGTTGTTTTGAGGTTTAGATT |
| | | ATTTAACACATGGAAAGCACTCACAGCAATGCCTGCCACAGAAAGAATATCCAGTACATCTTAGTGATGA |
| | | TCACCATTATTATTATCTGACTCCTGGAAAAGGACTTGATTTAATTCTCTCATGAAACGTTTTCTTGGAA |
| | | AACTGATGTCAACCAAGATTATTGGTCTTGCTGTTGCTTATAACACCCCAAAAACATGACTGTGTGGATA |
| | | AAAATATGTTGGAAGGGGTAGTCTTTCTGGGAGCCTGAGAATAGCCATGTAATAATAACTGCAAATATCT |
| | | ATAGTTACAATTTGAGGTTCAGGTAAATAAACTCTAGATCTTATAGAACTGCGGTAAGGTAGGATAGGGA |
| | | GACTCCTTCGACTTTCTCTGTTTATTTGTCTCTATTTTTAGGAGACTATGGAACCCCACTTCCTTGGTAC |
| | | TTTCTTCTACAAGAGTCGTATTGGCTTGGCGGTGAAGGTGAGTCCTTTAAAACACAAATCTTAATGTTTG |
| | | AAATCAACTCCTTGGGCTCTGTGCAAGATGTATATGGATCACAGAGGTGGCCCTCTATGTAAACGGTGTG |
| | | ATTCCTGATGAGTCAGCTGCCTCCTGGGGCTCTGCCCCTTGATGGGCATTGCAGCGTCTGGGGGACCACC |
| | | TTTCACAAGTTGCTGGGCCCTGTGTGATCATGAATGGCTGATCATGGATGAAGCCCTGGGTCCTGTACAC |
| | | CTTGTCCAGTAGACTAAATTGCCCTATTTAAAAAAGGCCAAGCCACTTCAGGGTTCAAAGAACTTTTGCA |
| | | GCTTTTCAGTATAAAGCAGAAATCCAGGGAATCATGAAGGAACCTTTGCATTCATCTCCCATTGCCTTCC |
| | | TTGTGCCTTTTTATTCTTCTCTGCCTTTTCAAAATATAAATTAGTTTATTCTCCCAAGATGAAGACTCCT |
| | | CCTGGGGCTGAGGCAGAGCTGTTATCTTCAGGGCAATACCTCAGATTCTCCTGGTGTTGATCTTTCTTAG |
| | | GGGTGGGGAAAAAGGCTGAAAGGGCATTTGCCCACAACACATCTTAGGTAAAAGGCACCTTTACTACTGA |
| | | ACCAAACAGGAGGCCTAGCTAGAGAAAGTTCTAGAAGCAGGGAAAAGCACAGACTCTTTTGTGAGGTCTG |
| | | AGAAAGCAAAGAAATTCCAGGGTGAAAGCGGGGGACTCCCCTAGAGCTGAAGTACTCTCCCATCTGTTTG |
| | | TTGCTCACCTACCTATTCTTTACTTTGTATTATTGGGCCTGGGCCAGGACTTATCCTGCAAGCACTGAGA |
| | | TGGATGTTTGTTTTCTCTGGGGGATTAGTCTTTTTTTTTCTTTTTTTCTTTTGTTTTTTGCTTTTGTTTT |
| | | CACTGGGTCAAACAAACAACACTTTAACAGCTCAGGATTTTTTCATTGTATTGACTTGTCTACCTGTAAA |
| | | CTTGTTAATTTTTTACTATAATAAAATTATCATATAATAAATGAAAAATTTCAACACAGGGCTTGTGGGC |
| | | ATTTTATTTTTCTCTACAATCCCAACAGATACTCTGCCTCTTAAGAAAAAAAGAAATCATAAGGAAAATA |
| | | TGCTCCTTCAAAAGTGAATCACAAATATGTTTGCCAACGGAAGGCAAATATTTTTCACCTGTCTCATAGG |
| | | CTGGACTGAAATGGATTTCTAAAACTCTCTAAAACCAGAAAAGAGCTGAGTGTCTCCACCCAACCTCCCT |
| | | CCTTTCACAGATTAAAAAATAAAAAATGGAGCCCAGGAGACATCCAGTATCTTCCCCTATTGGTCACCTG |
| | | GGACAAAATCTGGAACATGCACATGCATTGCCTGGCAGGAACTCATTCCAGTGATTAAACTCTTCAGGAG |
| | | GATGTTTCCTCTTGCTATTTCATTACCTATTTGTGCAGTTTGATAGCTAGTAAAGTGATCAAAGGAACTG |
| | | TGGGGCATAGATTCAAAAGTCCTTCAGGAAGCAGAAATAGAAGAACAGTACTAGAGGCAGCAGGTCCCTG |
| | | ACCAGCAGGCCCACTACCTGCTGCTCCAGCACACATCCTGCACATTTTCAGAGGGTGGGGGACAGAGGGG |
| | | CCCTGGGTGGCTGTTGCATTGAGAAATCTCGCCCTGCTCCTGTATGTGCACTTGAGGCCGAGAGCCCTTG |
| | | GATGCCTGGTGACAGTGGTTTCCTCCTGCCCCTGCCTTCCTCTCTGGCAGACTGACTGGCCCTTCTGCTC |
| | | CTCTTCCCCTTCCAGGATGTCCTGATATCTTTTTAAACCAAATGCCAAGTTTGCCAAAAAGTGTCTGTTT |
| | | GTGTGTGTGTGTGTGTGTGTGTTCAATGCGTGTGTTTATACCACACTTCACAATTTGTCCAGGCTTGTAT |
| | | TAATACCATCACCAGGCTCAACCCTGGTGTTAATTCCAAGATACTTAAATGCCCATCTAGGTGAATTTCT |
| | | CAGGTAAACCATATATTCAAGCTGTAGTTTAAGCTGGCTGCCCGTCATAGCACTTTGAATAGACTTTGTT |
| | | TTTGTTTTTGTTTTTTGAGACAGAGTCTCACTCTGTCGGCCAGGCTGGAGTGCAGTGGCACTATCTCGGC |
| | | TCACTGCAACCTCCGCCTCCCGGGTTCAAGCGATTCTCCTGCCTCAGCCTCCTAAGTAGCTGGGATTACA |
| | | GGTGAGCGCCACCCCACCCGGCTAATTTTTGTATTTTTAGTAGATACGGGGTTTCACCATGTTGGTCAGA |
| | | CTGGTCTCGAACTCCTGACCTCATGATACGCCTACATTGGCCTCCCAAAGTGCTGGGATTACAGGCGTGA |
| | | GCCACCACATCCGGCCCCTGAATAGACTTTTACTCAAGGTTCACCATGACTTTCACATGTTTTGTATTGG |
| | | AGTAAAATGTGCCAGTGGTGGGCTAAAGAAAATTAACTCATTTCAAATTCAAACCTGGTTTTCTTAATTT |
| | | TTTTAAAATCACAGTTTCTGAAACTGTGGGCTCCTCATGGCACATTGAGAGGAGGAGGTGAAACTCTCCA |
| | | AGTCTGAAGCTCCTGTTATAAATCTTCCTCTGGCAAAGATTGTGTGATCAGGCTTGAGTACCTCACAGTC |
| | | CTAGAGCAGGTCAAAGGCTGGCTAGGAAACTCATTTGCTCCCTGTACCTCTCCCCTCCTTTCCTGCCTTT |
| | | GCTCGTTCTCAGCTCCCGGTGGTAGAGTAACACTGGCTTCTGATTGGTGCAGGGTGTTCAACCAGAGAAG |
| | | AAAGAGCCCTGGAAAAGACCGAGCCCCTAACAGAGGAAACGGAGGATCCAGAGCACCCAGAAGGAATACA |
| | | CGGTAAAACCCCGATAAAGAATACACAGCAGAGGCGAGGAAAAGGCTCTAAGCACTGCAGAGGGCCAGAG |
| | | CAAAACATCTCATGGCAAGGGTGGAAAGAAGCCTAGGAAACTGACTCTCTCTGTGGACAAGTGTTAAACC |
| | | AGATCCCTTCTCAGAGGTCCATCTGCATGTGTGTGGAATGAATGGTTCAGCCCAGACATTAGCGCATATT |
| | | TCCTGGAGAAAGCAAATACCAACTATGTAGTGTGCCTGTGCCCTTGTTAGGCAAATCCCAAGTGAGTTGC |
| | | ACAAATGTGCTGACTTCCGAGGATTTAGCAAGAACAATAACTTTGGTCACTGGGACTTAAAGCGGATATG |
| | | AGCTATAAGGAAAGACAAAAATAAATGCTTCTGTGTCCAGGGGGAAAGAGACTCCAGGGGAGCTGACTAC |
| | | ACTTCACTTACGGCTTACAAATCTAGAAGGCCATTCATTGAAACCATCAGAAGCCTTTCCTGACAGTGGA |
| | | AGTTACCTAATAATCCCTAAACTGACGACCCAGATTTACAAGTTTTGTTTTCCTGGCTTTTGCTGCCCTC |
| | | ATCTTCTCTCTTAAACTAGTTCTGTATTTCTCCCAAGGCTTTTCATTCCCTAAGCATACGCATTTCTCTG |
| | | TGGCCAAAATGCTCTGGGTTTAGACAGGCAGCACAGCCCCTGGGCTCTGCCTGACAGGGCAGGAGAGGGT |
| | | CTGGCCTTTATCCCTCCAGCCCACCCCAGGGGCCATTTCATAAAACTAAAGCCAGAGACCTGCAGCCCCT |
| | | CCCAGAGTTAGACTGCAGTACACCATGCCTCTGGCAAGATCCTCCTCCCACAGTGGAAAGTCTAAGCCAA |
| | | ATCAGGAGGCTGGGGACTGGTTCCACCTCAGTTGCAGGCAAGGCCAGGAGGCACGGATAGAAGAAACAGT |
| | | GGACTTTTTCCCCCTAGGGAAAGAAATGCTTAGAGCTACAGTATTAAGATGACAAATTAAGCTGTGCCAT |
| | | ATAGGGTGAAATGAAGCAGGGATAGATGGGAGGTCAGGGAGAAGTGAGAGCACTCGGTGAGGGTCTGCAC |
| | | TGGAGGGGGCATGGGAGGAAGAAGGAGGGGAGTGGGGTTTGAGGGATGGTGATGAGGAAGCGTGGACTGC |
| | | CCTACCCACCTATTGGAAAACCTGGGAGTTCTGAGGAGCAAGAAGCCTTAGTCAAAGTCAACTCAAAGAT |
| | | TCAAGCCAAGGTGACTAAGAGAATGGCGGTCCAGAAAAGGTCATGGGAGAATCTGAAGGCAGATGTTGTT |
| | | TTGGGAAGATGAAGAACCTAAGCCGCTTCCAGAAATTCATGAGGAAATGCCCCGTGGACTGTTGGCAATG |
| | | AGGGCCTAGGACCAAGGTTGAGCTTGGGGCCAACTCTCCCTATAGACAGTGAGTGCATTCTGACAAGCAT |
| | | GGGCTCTGGGTTCAAATCCCAACTCTGCCACTCATGCCTATGTGTCCTTAATAGGACGCTTGATGTCTCT |
| | | GTGTCTAAGGTTTCCTGGACTATGGAAATGAGCCTAATAAATGTCTACCCCTTAGGACCATTGTAAGAGT |
| | | ACATTGAGGTAATTTGTGTAAAGCAGTCGAAGCAGTGCCTGGCATATAGGAGGTGCTGTATAAACGTTTG |
| | | ATGCTAGTATTACTATTATTATTCTGGAGTCTTCCTTGCAACGGTGATAGCCGAAGCCACAGGGGCAGGT |
| | | GACGTTATAGGCAGAATACAAGGGCCTGGAGACAGAGCCCTGGGGCCATGTAATTAGGCATTATGTTTAC |
| | | ATCATGTTCATTTTTTTTCCTCCAAGACTCCTTCTTTGAACGTGAGCATCCAGGGTGGGTTCCTGGGGTA |
| | | TGCGTGAAGAATCTGGTAAAGATTTTTGAGCCCTGTGGCCGGCCAGCTGTGGACCGTCTGAACATCACCT |
| | | TCTACGAGAACCAGATCACCGCATTCCTGGGCCACAATGGAGCTGGGAAAACCACCACCTTGTGAGTCTT |
| | | CCAGCAGAGAAGCTGGCTGCCATGCTAGCCTGTCATTTCCTGGCTTAGTCTTTCCCTATCAGCGGCTGTC |
| | | TACTCTTTCCCACAAATTTTAGTGACAAATATTTGCGGCCCCAAAAATGTGTAAAAGCTTTCTGCAGTAT |
| | | TCAAAGATCACTAATATGTATTCTCTTGATGGGGAGGTAGAATACGTTTATTGCCCCTTTTGTGTGCCGG |
| | | GGAAGTGGACATTCATTCAGAGAGTTGAAGTGACTTTCCTGAAGCCACCAAGTTGTCATGGCTCAGCGGG |
| | | GGCAAAAGCCAGGCACCACAGTTGCCTCTTGTTTCTCACACCTTGAGTCTTTCCCCCCATCTCAACAGTC |
| | | CATGGTGGTGATCAAGTCATGGCCACTGTCATCATGTGCATGGAAGCTATAGAGTCCTCCTATTTCCTTT |
| | | CTCTTTTCTTTTCTTTTTTTTTTTTTTTTTTTTTGAGATAGTAACCATTACCCATGCTGGAGGGCAGTGG |
| | | TGCGATCTTGGCTCACTGCAACCTCCGCCTCCCAGGATCAAGCGATTCTCCCACCTCAGCCTCCCAAGTA |
| | | GGTGGGACTACAGGTGCATACCACCATGCCCAGCTAATTTTTGTATTTTTTTTTTTTTTTTTTTTTTTTA |
| | | GTACAGACAGGGTTTCACCATGTTGGCCAGGCTGGTCTCGAACTCCTGACCTCAGGTGATCTGCCCGCCT |
| | | CAGCTTCCCAAAGTGCTGGGATTACAGGCGTGAGCGACCGCACCAGGCCGAGTCCTGCTATTTTCAAGGA |
| | | ACATTCCTTTTCCTACCAATCATTAGGCAGGCTTCAACATCAGCTGATGAGGGTTAGTGGTCGTTCTGGA |
| | | GAAAGTGAAAAAAGAATCAGTCTCTAGAGGGGCTTGTGGAGTAACCGCCTGGTAACAGAAGGTCAGGGCA |
| | | GGGAAGGCAAAGGGGCTCTGCGCGGATCTCTCAGCTCCGCAGGCGCCCCACTCTCCTCCAAGGGACCCGA |
| | | GCGCCATCTGCTGAGAGGAGAACACGGCCCGCCATGGTTTCCCAAGGAGCAGCAGACACGGACCTCGCAG |
| | | GGGGCAGCGAACCCACGTGACACAGTCTTCAAGTCCTTTGGAGAGCCCCAGGAAGGAACAACAGCGTGTA |
| | | CACCCTGTGATGGAATGTTCTCTAGGGCGGTTCAGTGTGAATGGAATGTGGGGCCGGTGCCATTCTAATT |
| | | GGTTCTGTTTCCCTCTAGTGGTTGATCGCGGAGATTTCGGCTTCTCCATCAGGACAAGTTCAGATAGCCT |
| | | GAGATGGTATCAGAACTCAGGGACAGAGCTGGGTGTGGCGGCCCTGCATCCATCTGCTTTCTCTCCATGC |
| | | TAACTGATATGGTCAGAGAGCTGGAAGCAAATTCCAGGACCCCAGGGCTCCGCAAAGGCAAACACATTAC |
| | | TTCATCGGCTGCTGACATGCAACTTCCCCCAGGGGTTAAAACAATGTTTAATACTAACAGTAATAATATT |
| | | TTTGAGTTTTACTTTATGCTGGCGCTGTTCTAATGTTGTAAGTGTATTAACTCATTTAAGCCTTACAACA |
| | | ACCTAAGGACATGGGAGTCATAGTTCCCATTTAAAAAAAAAAAAAAAAAAAGCCCACCATTGCTCTGAGG |
| | | CTTTTTATGTTTTGGATCCAAAGCTAATATTGGTGGTGGTAATTCCCATGCCTGGCTTCGATCAATTAAT |
| | | CAGCAAATGCCTAGGACTGCTTAGGGTTCTGGCCTTCATCAAGACCTTACCCGGGCTTTATGATGATGAC |
| | | ACCTGGCTTTTCAATAGCCATGACTGCTCACCCAGGAGGCAACGCCTCGAGTCATGCACCGAACACCTTT |
| | | TATTGATCCTCTCCAACACCAGGCTCCGTGATGGCTGAGCTGGGGACACCTGTGACTGCACGTGAACATT |
| | | TTGAGGCTGGGAATCCCAAAGGCCCTCGGCGTTGGCCTGGGAGCACCATGAAACAAGTAGAAGCAGAGAA |
| | | GGATGGCAGAGGTGGCCCTCTGCATTAGGGCCTGGATGTATACACTGGTGCTAAGGGGGCCCCACAGCTA |
| | | ATAGGGGTTTGAGTTTGACTGACAGCCCCAGGCAGGAATCTGTGAGAGTTCTCACTGAACCTGGTGTGGG |
| | | GGTGGCCCTCCTAAGGCATGTTGCTAAAGGCCATCTCTTCTGCCACTGACGCCTGTGTTCTGCAGGTCCA |
| | | TCCTGACGGGTCTGTTGCCACCAACCTCTGGGACTGTGCTCGTTGGGGGAAGGGACATTGAAACCAGCCT |
| | | GGATGCAGTCCGGCAGAGCCTTGGCATGTGTCCACAGCACAACATCCTGTTCCACCAGTAAGCGACACAG |
| | | GAACTGAGACCGCCCCATCCCCTCTCCTCACCTCTGCCCCCAGCACACTTCTCTAGAGCCCAGCTCAGGG |
| | | GTGCCAGGCCTGGGCACAGGCAGAGATACAGACTCTTATTTGGTTTCCCCTATGTTTAAAGTCCTTTGTC |
| | | CTACTTGCAGTGAGAATTGTCCCTGAGAATATGGGACTCTGCCTCTGCTGCTCAGAGCTGAGGGCTCCTC |
| | | CCTCAGAAGGGTGAGGCTGCCTTCGCTCTGACAGAGCAGCTGATCGATCCCCGAGCCCCTTGTGCAGCCC |
| | | TGAAGTACTTCCTCTCTGGGACCAAAGACAGGAGAACCATTGTTCCTTTTTCCTGTTGAAGCCACGGCCT |
| | | GAAAGGCAAACTTTTCAGGGGGCTTTTCAGTTACTTTTTTTCCCCAATAAGATATCTTTTATTTCTTATC |
| | | TAAGAAGCTACGCATAGTCATTGTGAAAGAAAAAAAAGGAAGGGAGGAAGGAAGGGAGGAAGGAAGGAAG |
| | | GAAGGAAGGAAGGAAAGAAGGGAGGGAGGGAGGGGAGAAGGAAGCGAGGGAGGGAGGGAGGGGAGAAGGA |
| | | AGGGAACAGGAGGGAGGAAAAGGGAAGGGGAAGGAGGAAGGAAAGGGAAGGAGGGAGGAAGTAAATATAG |
| | | GTAAACAAAAAATTGAAAATAAAAGTCACCTGTAATTTCACTACTCAGAGATAACCGCTGAGTTATAACA |
| | | TTGGTATATAATTTTTTAGAACTTTCTCCTATACATGTATAGATAGATAAACACATATACTTCAAAATGA |
| | | TAAAGAATAGTAAAACTATGCATACAATTTTATAACCTGACTTTTTTTTCAAAAAAAAGGATTGCTTTTT |
| | | TAAACATAAGATATCAGGAACATCTTTCATGTCATTACATATTCTTCTATAAAATAATATTTAATGTTTA |
| | | CAGATTATTCCATTGTATGCATGAACTATGTAAGCCATCCTCTTATTAGATATTTAAGCAGGGTCTGCTA |
| | | TTTTTGTATTGTATCATAAACACCACCACAGTGAGCATCTTGATTGCCAAATCAAGAATACTTGTCCTCA |
| | | ATTATTTCTGTAAGATCAGCTGCTGGAAGTGGAAGTGCTAAGCCACTGCTTTTCTCGTTGTCCCATCCTC |
| | | CTAGCCTCACGGTGGCTGAGCACATGCTGTTCTATGCCCAGCTGAAAGGAAAGTCCCAGGAGGAGGCCCA |
| | | GCTGGAGATGGAAGCCATGTTGGAGGACACAGGCCTCCACCACAAGCGGAATGAAGAGGCTCAGGACCTA |
| | | TCAGGTGCTCAGAGCTGGATGGAGACAGGGCCACAGATGGCAAATCCATGGCTCCCCAGTGCACCCAGGA |
| | | GGCAGGGGAGGCTTGGAGCAGGAGAGCTTCTAAGGGTGGGAACACCTCTGTGAAGTTACACCAAAAATCT |
| | | AAGAGCAGCCCCCAGATCATTTTCCCTGCAGAGCACTGTCTCACAGCAGCCTGGGTTTTATTTGTCCTGA |
| | | GATTGATGTGCTTGAACAGTCTTCAAAGGGTCTGATCCGAGGAGGTGAGGGTTGCCCTTTCTGCATTTAC |
| | | AAAGCCTGAACAGTATTAGGGCTTTGAACGCTATAAACATCTAAGAGGCAGCACCAAACCACTGCTGGGT |
| | | TAAGGTACCCCCACAATGCCACTTGCCCTGGGCCTTTCTCTTCCTCACCCTCCACAGCCCCTTAACTCTC |
| | | CCGTCCTTCTTGTGCCTCCAGGTGGCATGCAGAGAAAGCTGTCGGTTGCCATTGCCTTTGTGGGAGATGC |
| | | CAAGGTGGTGATTCTGGACGAACCCACCTCTGGGGTGGACCCTTACTCGAGACGCTCAATCTGGGATCTG |
| | | CTCCTGAAGTATCGCTCAGGTAACAGCTGCTGCTCAGTCTCCTGGGCTGGGCTCTCACTGCAGCCCTAGC |
| | | TGTGGTCCCCACTCTCTCACCTGCCATTTTGTAGCTGAGTACAGGAACCACAATGACTACACTCAGAAGG |
| | | GGGTTTATCAGTGACTTGGTGAATCTAAGTTCCAGCTAAAGCCTCCTGAGGTTTTTACAAATATAAACAG |
| | | AGAATCACTGATGATGCAACCTACTTCCCAAAATATTTTAGAAAATTCTCTTGACCTGCAGCCCTTCTGT |
| | | CTGGAATAATGGATGCTACTCTAGGTGAATGTCTTCTCTGACCATGGGGACCCAGGTCACCTGCAAACAT |
| | | ACCTAGAAGCTCCATAGCTGTCAGATGACCACTCAGGACCAGTGTGAGGGTGACCTGCTGGGCATTCAGT |
| | | GCTCCAGAGGGTGGCCACAGATGGAAGTGGCTCCTCTGTCATGGCACCTCTCAGACAAGGGGCTCAGATC |
| | | AGAAGAGACAGCAAGCAGAGCTGAGTGCCCATAGAGGTAACAGCACGGTTCAACCCCGTGGTCAAGCCAG |
| | | AGCTTTCCCCCTTGCTCTACTCACACAGCGTTGCCCCGTGCCTTTCTCTGAGGGTTTGTCATCCTGAAAT |
| | | CCTCATTGCTATTTTCTTTCTTTCTTTTCTTTTTTTTTTTTTTTTTTTTTTGAGACAGAATCTCGCTCTG |
| | | TCGCGCAGGCTGGAGTGCAGTGGCGCAATCTCCACTCACTGCAAGCTCCGCCTCCTGGGTTCGAGCCATT |
| | | CTCCTGCCTCAGCCTCCTGAGTAGCTGGGACTACAGGTGCCCGCCACCACGCCTAGCTAATTGTTTTTGT |
| | | ATTTTTAGTAGAGACGGGGTTTCACCGTGTTAGCCAGGATGGTCTCGATCTCCCGACCTCAGGTGATCCT |
| | | CCCGCCTTGTCCTCCCAAAGTGCTGGGATTACAGGCATGAGCCACCGTGCCCGGCCTGCTGTTTTCTGTT |
| | | AATGACATCTCCAGTTAGTGAGAGTATGCACGTGTGTGTTCTTTATGAAGAGTATAAATCCAGAGCTTAA |
| | | TGATCCAGAAAATGTACATATGAAACTCCCTAGATGCTGACCATAATACATGAGCCCCTAATATAGAGAT |
| | | TTATTTGAATCAGATCCTATGCTGGATACAGAGACACTGTGTGTGGCAATGCTTTACAGTATGTAGGAAG |
| | | CTATGAAATGTTAGTTATTATTGTCCTAATATGCTGGAATTTGCTGCTGAATTAGTTCCCTTGGGTTTTT |
| | | TTTTTTAGTTAACTCCTGATTTTTGCAACTATATAGCCAGGAAATTGCTGTACACCCTTTACCAACAATG |
| | | CCCAACCCAGGGCAGGCCTGGTGATTGCCCTGGCCCCTACCTTGCAGGCAGAACCATCATCATGTCCACT |
| | | CACCACATGGACGAGGCCGACCTCCTTGGGGACCGCATTGCCATCATTGCCCAGGGAAGGCTCTACTGCT |
| | | CAGGCACCCCACTCTTCCTGAAGAACTGCTTTGGCACAGGCTTGTACTTAACCTTGGTGCGCAAGATGAA |
| | | AAACATCCAGAGCCAAAGGAAAGGCAGTGAGGTAGGTGTCTGCCCAGGGAAGGACCCTGGCCTGGGTGAG |
| | | AAGGAGCACACAGCACGGGGCTGCCACTCCAGACATGGCTACTCACACAGGCTCTCGCCACCAGAATCAG |
| | | TGTCTTTGTTCTGGGACCATTTGCAGAAGATTTCGATGAACACATTCTGAAGCCTCCTCCTACAGAGATG |
| | | CTTTAGCCAAAATGAAACAACTAGCTTTAAATGGTCTGCAAGTATTACATGCCAGATTACACACCAGTTT |
| | | GGTGCGGTTTGGTGCAACATAGAAGTGAGTGTCTTATTCTGTAAGGTTAGGCTGTTTTAAGAGCAATTGG |
| | | TTGAGCTTCATTTCAACATTAATATTCCCTAATTAAACCTGAATTTCAGTGGTAAGTGAAAACTAAGAAG |
| | | AGGCCTCCTTGGGTGCTATAACATAAAAATGATGAAGGCAAAAAGTACCAACCAGCAGAGACCACTTCAG |
| | | CACATCAGGAGACCCAGTTTTATGTCTGTGCTGCGAAGTGAACAAACTGTGTCATCCTAGGCAAATTATT |
| | | TAATTCCTCCTTTTTTTTAGTATTTTTTTCTTCTTCACATGGAACATGAAGCTAATGACCTCTGCTTCTA |
| | | TTTCTTAGGGATGTGAAGATAAGTGAGATAAAGTATTATAAATGTGCTCTGGGCTTCTTAAGAACAGGCA |
| | | TTGCTCACATTCAAATGGTCATGATTATGATATGGCAGCATTATTTATGCCTCTGGTTTAAGTGTCTGGC |
| | | TGCCGCTGGGGTTTCCTATGTCCATCCACGGGGAGGGAGGCACAGAATGTCTCCCACAGGCAGAACCTAC |
| | | AGCTGCCACATAATTGATGACAAGCCAAAGGGACCCTTGGAGGTTCTGCTCCTCTCTGTGTGTGACTCAC |
| | | ACACTCTCTAGGATAAAATCAAGCGACTACACCCTCAAAATGCTCAGATGAATTAACAGATTAAACAGTG |
| | | AAGAAAAAAATGTGTTGACTACACTTGGCAGTGAGAAATAAATAAAGCGGGCGGTGACAGCAGCTGGCAT |
| | | CAGGGAGAGGCTGTCATGGAAGGGATGTGCATCTTGTCAGTCATCCCATCCATCTGTTGCAGGGGACCTG |
| | | CAGCTGCTCGTCTAAGGGTTTCTCCACCACGTGTCCAGCCCACGTCGATGACCTAACTCCAGAACAAGTC |
| | | CTGGATGGTAAGGACTGGACGGGCCATACTTGGGTTCCGTCTGGCAGCCATCTCCCAGTATTGCTGGGTG |
| | | TGTCCTGTTGTGATGCATTTTAATGGGAGCAAAGAGAACACTGGGTACTTCTGCAGGTCACACAGTTGTT |
| | | CTTTTGCTTTGAGCTTCTTTCTCCTCTTCCTTCTTCCTTCATTCCCAAAGGGATTTTAAAAGTCATGCAC |
| | | CTAAAGGCCCTCTCCCTTTAATGAGGAATACACTCTGTGCTCTTACCCTTAGTAAGCCATCATTCCTGGG |
| | | GTCCCCCTGCCCTGGCTCCAGGCCACATTCCTTAGTGTCTGGGGAGAGCTTCTTCTACATGTGTGCCGTG |
| | | GCGCCCTCTAGTGGAAGCATGGTGATGCACGGCTCTTCCAGTGAATTCGTGGAGTCAGAGATTGCACATG |
| | | TGGATGGCAAGTCTGGAAATAGCATACACCCCTGTTATACTCCTGATTCTCCCCTCAGCTTCCCAATTTC |
| | | CCAGTGATTCTCCCTTTAATTAGGATGCACTGAAGCTCTCAGGGGTGCCCCCATCTCCAAGGAGCTGCAG |
| | | TGGAGAGGCTATCCCCTCTCTATGTGAGAGAATGTGTGAGAAGCGTATTCCCACACAGGAGCAAAACTAA |
| | | ACTTACGTACTGATGCAGGTTAATGAATGGGGAAAGTATCTGCTTATCAAAGAAAAGGCATATTTTTCTA |
| | | TTTAGCACAAACTTTTTCAAATGTTAAGAATTTACTAACTGAAATCTGGTGAAGCAAGAGAACCGGGCAA |
| | | TATTTGCGTTGTCTGATCATTACAACTGGAGGGAACATGCTCAGAGAGGCATCATCACTGTTCATGCACC |
| | | TGCCCTCTCTTTACACTGAGAGACCCTGTGATGAACAGAAAACATCTTTTTAGGATGACATCTCTGGGTC |
| | | TTTCTCCTAGCCTGCCTTGCTGTGGGTACCTATCTCCCTGCTCTCTGAACCTTGGTCAAGAAGTTTATAT |
| | | TTGTTTTAAATTGATACTAATATGTTAAGTTACTGTGATTTGCCAAAATCAGATTGGAAACAGGGCCTGC |
| | | ATGGCTGAATGATTCTTTTTTTTAAATTACTTTATTTCTAAATAAAGGTTTTCTTTGTATAGAATCGGGA |
| | | TGCTGTGAATGGTGGGAAATGCACTAAATAGTTATGCCCCAAATAAGAAAGGGAAAATCATTTGAATCCC |
| | | CAGTTAGCTCCTTGAAAGTCTTTTCACTTAAACACACCCACATACCACACACACACTCACAGACCTCCCT |
| | | CCCAGATGCCCAAAGCCCTGCTGACCTACAGAGCTACTTCTGGAAAGGCTGACACATGCCTAAGACACAA |
| | | TTCCTGGGAATCCAGCAGCTTTGGGTTCAATTTCCTTCCTAAAAGAACAATGAATATGACCCCTGGAGAG |
| | | CTATTAGGGCAGAGCTGCTTCCTTAACGTAAAGGACTCTCCAGCCTCCGTATGAAGTCATCTCAGAGCTA |
| | | AAGACAATCAAGTCCAACTTGCAGATTTGACATAAAGCAAGACTTCCAATCCGGCTAGGCAGAAGGATTT |
| | | TGGTTGAAAACCATGAAATCCCTTCATATGGATCATTTTTTAAACAACAAAAAAAGAAAAGAACCTACTG |
| | | GGTGTCCACAACTCTGAGAGCTGCTTTCTGAAGAGTCATGTTTTGAGTCCTGGAATCCCTCTCCCTTTGA |
| | | CCTGCCTCTCAAGACAATGTGCGAGAGAACTCTCTCTTCAAGTGCATGCAAGTGAGGTTTTCACAGTTAG |
| | | ATTTTTAATTTTAAAGTAATACACATTTGTACATAAAATTCAATTCTGACTGTATACATGTGTCAGATAA |
| | | ACAGTTGATACCTGACACTTGTTCACAGTCTATGATACGCACCGCATATCCTACCCTCTCCCCCAGCCTC |
| | | TCTCCATGGCTTCTCAACCCCCCCTCTGCATTTCCTGTGACCTGAGGATTCAGTTTTGTTTGTGGAGGCA |
| | | GGTGCAATCCCAAGAGAAACTGTGCAATCTTCTGAGAAGTTAGAGTAGGCATGTGTGTGTGATTTAGGGA |
| | | AGGTACTTCTCACTCAGCTTGGTCACCGGTTCCAGGTTTGTGTCTTGGGCAAGTCCCCCATAGCTGGTGA |
| | | CAGACCAGAAAAATGAAAACAACTTTGACTTAGCCCTCAAGTTTTCAGTGAATGAGAATGAAAAACAACC |
| | | ATGAGTAAGAGATTTCTTACCGAGATGATGTAAAGGATAATAATAGCAGCCAGCACTCACCTATGTGCCA |
| | | GGTATTTCTCTAACTGCTTTGTGTAGTTTGACTCATCCAGTCCTCAAAAACAACAATGAAGTGGATACCA |
| | | GTATTTTCCCCTTTTCACAGATGAGGAAAGTCTAATGTGACCCACCCAACATAACATAGTTTGAGGGGAC |
| | | AGAGCATTTCGTTGAACAGAGGAGGAACTGGCACAGGAAAGTTGCATGACCCCCCCACCAACCTCCGCCC |
| | | CCAGGTTGCACAGCTAGCTAGTCGGGAGGACTTTGCTTCCGTTTCCCTCTGCCTCTCAATGATGATCTCA |
| | | GGGCCAACTAAGCTAAAAGCAGACTTGATGGAGCATCAGTCCTCTGAAAGAGTCACTGCCGAGATACAAA |
| | | ATACCTCTTCTTCAAAGGGGAAGTGGAGAGAAGTAGGAAATCTGGGTAACCTCACAGTCTTCCAGTTTCT |
| | | GGAAAACAGAGCTGGCATCAGTCTTTTTTCTTGTCCTAGGGGATGTAAATGAGCTGATGGATGTAGTTCT |
| | | CCACCATGTTCCAGAGGCAAAGCTGGTGGAGTGCATTGGTCAAGAACTTATCTTCCTTCTTCCAAATAAG |
| | | AACTTCAAGCACAGAGCATATGCCAGCCTTTTCAGAGAGCTGGAGGAGACGCTGGCTGACCTTGGTCTCA |
| | | GCAGTTTTGGAATTTCTGACACTCCCCTGGAAGAGGTAAAGTAGAGATTCCAGCTGGTTTCTGTCAAGTG |
| | | CCAGAAGTGGCGGTTCTTTGAAAAAGTCTAACATTAGAGCAAAGTTTTGTAAAAGCAAAAAGCCATCGTT |
| | | CCCCACCCAAGCATAGCAACTATCTTTATTTTTGGCATAGTTCCCCCATCTCTGCATGCATACAAATTTT |
| | | ATGTACTTGTGGTTACTGTGTGCTTACGTTTTTGTATTTATAGAAGATGATGTTCTCAGATAGAGTCGTA |
| | | ATGGATTTTCTTCCCATTATGAAGCAATACCCAACAAAACAGAGCTTGGGTTAGATTTTTCTGAGAATAA |
| | | GAATGACTAAACAAAATTCTCTCTTTTTTTCTTCTTGACAGATTTTTCTGAAGGTCACGGAGGATTCTGA |
| | | TTCAGGACCTCTGTTTGCGGGTATGGTGCTGGAGCCAGTGGCTTGTTCCCTTCCTTGCCTCCCTCCCAAG |
| | | TTCCATCTCGAAAGTCTAAGGGGCTGGGCACAGTGGCTCATGCCTGTAATCCCAGCAATTTGGGAGGCCA |
| | | AGGCAGATGGACCACCTGAGTTCGAGACCAGCCTGGCCAACATGGTGAAACCCCATCTGTACTAAAAATA |
| | | CAAAAATTAGCTAGGTGTGGTGGCGCGCACCTGTAATTCCAGCTACTCGGGAGGCTGAGGCAGGAGAATC |
| | | ACTTGAACCTGGGAGGCAGAGGTTGCAGTGAGCAGAGATTGTGCCACTGCACTGCAGCCTGAGCGACAAG |
| | | AGCAAAATCCATCTCAAAAAAAAAAAAAAGTCTAAGGAAAAAGTCATGAAACAACAAAGCAGGCAAATAC |
| | | TCCTCCATAGTATCTGACTCCCCAGTAGTAGGCATTTTGCATCCTAGATGGCTTTGAGTGACAAAGGAAT |
| | | AACAGACTGAGTTAGGTCTAGATGGGGACACTTTGGATGAATGAGGATTCTTACGGAGGTCAGGTTGGTA |
| | | GCTTCATCCCTCAGCTCCTCATGCTGTATCCCCAGTCTCTCGGCCTGCCATGTCATCATCCTCATCTCCT |
| | | CCTGTCATCTCCACCAGGCCTCTGATCCATCTCTGTCTGCATGAGTGACAGCTGGCAGAGTCCTTAATGT |
| | | TTATCAAATACAACTCAGACGTCAGTCTCCTGGCCCCTTTGAGATCAACATAAAATCATTTTGAACCCTT |
| | | ATTTAGTGGTCTATGGGCTTTGAAAACATGGGGACCAAAATTCCTGTGGATTCTAGAAGTCTCTCTTCTA |
| | | CATGTGTCAGCCTGGGCACCAACTAGCTCCTTCCATGAACTTTTATCAAACCCACAGCCACACAAAGCAT |
| | | GTGTGAGTGTAGCAGAGTTTACAGCAGAGGGTGGAGGGTGGGGAGATAGATGTGTGGAAGGGTTACCTGC |
| | | CACACAAACAGAAACCACTTCTGATAGAACACGAGGTGTCCACCCACACTGTAAAATCCTCTCCTGGTAC |
| | | AGGCAAAGCTTTGCAGCGATTCTCCTTTGCTGCCCCTGGGCTCCTAACACCTCCTAAACCACCAGTTACC |
| | | TCCTTCTTTCCAGTGTGGCATATTTCAGTGTTTTCCTGTTGGAGTGTTTCCTTTCTATGTGGATTCTGGA |
| | | ATCAGCTCTTAAGATAACTTGGTTTTCATCTTTCTTCATAATGATCCCAAACATCTATCTACTATGCCTA |
| | | GAACTACCAATGGACACATATACCAGCCCAGATATGCTTCAGCCCATCCCAGTACATCGCATGGTGACCA |
| | | AAAGATGTAGTCGTCCTGGCACAGTGGGTGTGGGGCAGGAAGCAGTCCTCTCCAGGGGACAGCAGCAATT |
| | | CACCACAGAACCCAAGTTTCTTTCAAGCTCTGCTGACACAGAAATTGAATAATCTCAGCTCACCCAATGT |
| | | CAAAGACTCATATTAACCAAGACCAGAATGAAAATATGCTAATTTATATCAGAAGCTTTGCTGGATTCAA |
| | | GAGTTAGGGCCTTTTACCTGTGCAGAATATTCCTTCTTGATAAATAGGCCCTCTCAGGAGAATAAATTAC |
| | | ACATCAGAGGACTGTTTAGTCAGCATAGGCATAGAACAGGATGTTCCAAAGATACAGTCAAGGGGAGTGG |
| | | GTAAGAGTGTAGCCTCTGGAGTGAGGCCGACCAAATATCAAACCTGAGCTTCATAATTTGCAAACTAACT |
| | | GGCTTTGGGTAAGTACATAGCCTCTTTGTACCTGTTTCCCCATCTGCAAAATGGAGATAATAATAGCATC |
| | | TACCTGTAGCATTGTTGAGAGAATTAAGTGAGTTAATGCTTGCCGACTTATAACACAGTATACGATCACT |
| | | GATTAAGACTTAGCAACTCTAAACTAAATGTTTACAAACCATCTCTTACCTCAAAGCACTTAACATCCAT |
| | | TGTCTTATTTGATTATCACTGTAATCTTATGAAGCAGGCAGGGCAGGGGTCTGCCCCATCTGGGGGGAAC |
| | | TGAGCTCACAGAGGTTGGAGGGTTTGCCTAAAGTCACCCAGGCCACTGGGTCTCACTCTCTGGTCTTAGC |
| | | TCTGTAATCTAGGATGCTCAATGCCACACTCTCAGCCACTTTTCAGATGGCTAAGTACATTTGTTTTGAG |
| | | TTAGCTCAGTCTCAGAGGATGACATTTTCTGATCTTGTCTCCAGTGTTTAAATGAACCTGTAGCTGTGCA |
| | | TTGGGGTCACACAATGCGTGGCATGGAGAGGGTCTGTGGCTGACTGCCACGGTTACTACGTGAAACCATC |
| | | ATTACAGCAGTTACTACTGTTACTGCCTGAGAACATCATTACAAGACTGAACGAAGGGATCAACATGGAA |
| | | ATGATAACAAAAAAACCAAAGTAACTGTTTTAAGGAAAGGCTAGCATCGGGAAGAAGAAGAGAGAAGAAG |
| | | AGAAGAAGAAAAGGGCTCCCTGCTTCTAATGAGTAAAGGCAGCTCCCTAAGCTTCTGCAGCCCTTCATTA |
| | | TTTATTGGGTAACAGGAGGAAGGAGCAGGAGGTAATGATTGGGTCAGCTGCTTAAATGATCACGGGTTCA |
| | | TGTTGTTACTGACAGATTTCAATTATGCCTAATCATAAGAAACATTTGTGCAGCCTCCAACAAGGGTCAA |
| | | TGCCACTTCTGAAGGGGTGACTCATAGTCAGTAACTAGAAAGCAGCAGATAGCTAGGGACAAACTGGCGA |
| | | TTCTGAATAGGCCTGGAACCCTTAGCTCTGGCCAGGTCAGTGGGCTCCAGTCAGGATGGAGCCTTCAGGG |
| | | AGAGATCAAAGCTCAGAGGTTTGAGATGATATCAGCCAGCAAAGAGGAGGGGCAGTAGGGATCCTCCCAG |
| | | AGGGAGGGCCAGCCATAGAAGACATCAAATCTGAGCCCGGATCAGGAGAAGGAGCCTGCAGAACTGGGGC |
| | | TCTGGCACCGAGAACCTGCAGAACTTCGCCCCTCTGAGTGCAGGTGCCAGGGCTGGGGCTGCCACCCAGC |
| | | CTTCGCATCCCAGGCCTGGCACGTCATAGGTAAATGTAGTTGAAAGGATGACTGAGCTGATCCAATTCCC |
| | | TTTACAACTGTCCTTGTCCTGGGGGACTTGAGGAGGGTTAAGAAAGCAGCTGGGGACCAACCAACAGTCC |
| | | TCTAGGCTCTCCATGTCCAGCAATAGTTGTTCAGCAAATGAGCATTAATCAGTGACTATAAACTGTAGCT |
| | | TCAACATAACCGACAACTTGCAATGGTTTCTAGAGCATGCTCCCATGTGTTATCTCATTTAAATTTCCAA |
| | | ACCAATCCTGTGAAATGTTCTTTTTTTTTTTCTTTTTTTTTTTTTTGAGATAGAGTTTTGCTCTGTCACC |
| | | CAGGCTGGAATACAGCGGCTCGATCATAGCTCACTGCAGCCTTGACCTCCTGGGCCCAAGGGGTCCTCCC |
| | | ACCTCAGCCTCCCAAGTAGCTGGGACTACAGGCACACGCCACCGTGCCTGGCTAATTTCTTTTCTAGTTG |
| | | TTTGTAGAGACAGGGTCTCCCTATGTTGTACAGGCTGATCTGAAACTCCTGGGGTCAATCAATCCTCCTG |
| | | GCTTGGCCTCCCAAAGTGCTGGGATTACAGGCATGAGCCACCATGCCTTCATTTTACAGATAAGAAGTCT |
| | | GAGAAAACTCAGATTTAGGCAGATTGAGTCACTTCCCCAAATTTATGTATCTTGTAAGAATCCATATTCA |
| | | AACCTCAGTCCCCTAACTCTTAGTTCATTACTTTTTCTACCACTTCTCAGTATCCTCTAAGAATTCAGAA |
| | | AGAACCACATCGACTCTGATTTTTCATTTGTTTAAGTACACAGGTAATAGGTGAATGTATTTTGTTGTTT |
| | | AAAAATTCATATAATACACAAAAGGCTAAAGTCTCGCTTCCCACTTCCTCTCCCCTTTCTACCCAACTCT |
| | | GCCTCCCCAGGGAGAGCTTCTGCTGACAGTCGGTGGACATTCTTTCAGAGTTTTACAATTATGTGTGTGT |
| | | GTGTACATAAGATGTCAGTTTTTCTTTGTGTAGGATACATGAACATGAATTTTAAACATAAATGTGAGTG |
| | | TATTACACATATTGACCAGCACCTTAGTTTTTTTGTTTGTTTGTTTGGTTTTCTTTGTGCTGTTTGAGAA |
| | | GGAGTCTTGCTCTGTCACCCAGGCTGGAGTGCAGTCTTGCAATCTCGGCTTACGCAACCTCCACCTCCTG |
| | | GGTTCAAGTGATTCTCCTGCCTCAGCCTCCCGAGTAGTTGGGATTACAGGTGCCTGCCACCATGCCTGGC |
| | | TAATTTTTGTATTTTTGTAGAGAGGGGGTTTCACTATGTAGGTCAAGCTGGTCTCAAACTGCTGACCTCA |
| | | AATGATCCATCCACCTCAGCCTCCCAAAGTGCTGAGATGACAGGCGTGAGCCTCCGTGCCCAGCCAGTTT |
| | | TGTTTTTTTATTAACCAAGTTACGTATTTTAAACTTCTCCATGTCAATGCTTTTAGAGCTATTTTGTTCT |
| | | CTTTAATGTTAATAGAGAATTTTAAGGCAATTTCAGGTGAATCTATACAATTTCTCTGTATAAGTAATTT |
| | | ACACTAGAAATAGATTTTTATAAAGATGATTAAGCTACCAGCCTGGTATTTCATTGCTGACTTAAATGAA |
| | | GAGGAAAATCAATGCTGTAAGGGAAAAAAAAAATGGCATTAGAGATCCAGACCTTATAGGCATTTTCCAA |
| | | ATTATTAATTCAATCTCTCAAAACAGGTGGCGCTCAGCAGAAAAGAGAAAACGTCAACCCCCGACACCCC |
| | | TGCTTGGGTCCCAGAGAGAAGGCTGGACAGACACCCCAGGACTCCAATGTCTGCTCCCCAGGGGCGCCGG |
| | | CTGCTCACCCAGAGGGCCAGCCTCCCCCAGAGCCAGAGTGCCCAGGCCCGCAGCTCAACACGGGGACACA |
| | | GCTGGTCCTCCAGCATGTGCAGGCGCTGCTGGTCAAGAGATTCCAACACACCATCCGCAGCCACAAGGAC |
| | | TTCCTGGCGCAGGTACTATTGTCGGTCGGTGTTTAGCTGAGCTCAGTGGCTCCTCTCCCAGCCTTCCCCT |
| | | CCTCTCCTGAGTGTTCCTTCAGGCATGGGTTATAACTCAGCAAGGAGCACCCTCTTTAGATTCTGCTGGT |
| | | TTTGTTTCCTGCTTTCCAAACCCTTATCTTGATTCTTGGTAACATGAATCTTCTTTGTAAGTTGGACCTC |
| | | CCCTAGCAAAGAAAATAGAATAATAGTGAAAATGTTAATATTGTTTTTATTTTTACAGTGAGGGATAAAG |
| | | TCATGTTTTCATTCATTTTTGCAGTGACCCTACATATCAAAATCATTGCCCTCTTTTTTCTTTTAATGTT |
| | | GTTTAATTTAGAAAAAGAAGCTCTGGTTTAAAGAACAGTGAGTCACGTGACTTGCTCTTTGAAATGCCCT |
| | | TTGAAGTCTGGCTGAACACTGGGCTGCATTCAGATTCTTCAGTGGCCACCAGAACATTCTGTTTTCTTCT |
| | | GCACATCTTACCTTTGCACACCCTGCTTATTATGTTCCCCCAGAAGCCCAACCCTCTCCACCAGGGGCTG |
| | | ATTAGGAGGCTGCAGGATAAATGTTTAAAAGAATGAAGATGTGTGTGCACGCGCACGTGTGACATCTCCA |
| | | TGCCACAGTCATGTTTATTCCACGTCTATTCTCCCACAGATCGTGCTCCCGGCTACCTTTGTGTTTTTGG |
| | | CTCTGATGCTTTCTATTGTTATCCCTCCTTTTGGCGAATACCCCGCTTTGACCCTTCACCCCTGGATATA |
| | | TGGGCAGCAGTACACCTTCTTCAGGTGCGCGGACTCGGGGTCACCATTCTCCTCTGTGGGTTTGGGGCAC |
| | | CTGGGTCACATGCTGCTTAGAAGGGCCCTGACCTTCCCACTTCACTGGGACCTTCACCAATGAGAGAGGG |
| | | GAGGGGTCTTTGGGCTGCCTGCAGAAAGGAACTTAATGTATCTGCCACTGCTTGGAAAGGCGATCCTAGT |
| | | GGACAGGCAGGACTGCTTGGGAAGGCCGAATGGGGAAAGGAATGCAAAGCTTAGGTGAATGGGTTGAAGC |
| | | GCCATCTTTTTGAGGCATAGGTGACATGCCATCAGACCACTGCGAGTGTTCAGGCAGCCTACCGCACTCC |
| | | CAGGAGAGCTAGCGCCATCCCAAGGCAGCATTCGGTGCCTCCAATACATACCTGGCACACAGCAGCTATC |
| | | CAGTAAAGGCTCTGAGTTGCATGATGTTGGCACGCGCCTGCTCTGTCCCAGTCACATGTCTCACTCTGTC |
| | | TAGCATGGATGAACCAGGCAGTGAGCAGTTCACGGTACTTGCAGACGTCCTCCTGAATAAGCCAGGCTTT |
| | | GGCAACCGCTGCCTGAAGGAAGGGTGGCTTCCGTAAGTGCCTACGCGCCCCTGTCCTAAGAAGACTAGCT |
| | | CCCCTGGGAGGACCCAACGGTGGGTTCAAGATGGCAGGCGTTGGGGAGGCCCCACTCAATCCTGCTCTGC |
| | | TGGTCACTTCCATGTCTCTGACCAGCACTCCCCCAACCTCTCCTTCCACACTTGTGTGCAGGGACATTCA |
| | | CTACCTCCTAGGAAGCCCCCACACCACTGGACAGCTCTATATTTCTCAGCATAGAAGTTCTATGTTGAGT |
| | | TGACAGATGATTCCCCATAACTTATTTGAAAGGCCTCTGAGCAGGGAGGGAGGGAAATAGGGTTATGCTA |
| | | TTGTGTGATTGGGCCTTGAATGGCGTGAGTGACACAGTGGCCAGTACTTTGTGATAGTTGTGAGTCTGGA |
| | | GAAGGGAGTTAGCGAAGGCCATTGACATCCACCAGGAATCCTAAAAGTTCAATATAATTTTAACTTTTCT |
| | | CCCTCAGTCTTTTTCAAAGCTGTCAATAAGGACCAAAACAGACTAATTTCAAATTCCTCTTCTGGTTGCT |
| | | GTGTCTCTCAACAGCTAGAGCTGCTAGGAATAAAAAGGGAGACAAAACGATCCACAAGCTAGAGATGGTT |
| | | ATTCCCCAGCCCCACACCTAGTCAGTCACAAAACCCTAGTTTTGATATTGCTTGAGCAGAAACCAGCCTC |
| | | CAAGAGAATAAGAAGAAAGGGCCTGGGTCTAAAGAGGAGGAGGAAAGGGTTGGGCACAATTTCTTATGCC |
| | | TAGGGATTTGTCAGCAACTTTGAGGCTGATTATGGAATATTTTCTTGTCTTCCATGAGGGAGTACCCCTG |
| | | TGGCAACTCAACACCCTGGAAGACTCCTTCTGTGTCCCCAAACATCACCCAGCTGTTCCAGAAGCAGAAA |
| | | TGGACACAGGTCAACCCTTCACCATCCTGCAGGTGCAGCACCAGGGAGAAGCTCACCATGCTGCCAGAGT |
| | | GCCCCGAGGGTGCCGGGGGCCTCCCGCCCCCCCAGGTACCTGACCTCCAAACAACGGGGCCCCAGGTCTG |
| | | CCTGCCACAGAGGGACTAGGGGAGTCCCTGGTATCTCCTGAGTCTCTCACAAACTAACATTTCAAACTGG |
| | | CAGTTGAGTAGGGGACTAAACCAAACTCCCTGCACCCTCTGGGAGGGGCTCCCCACAGGGCGCTGTGGCT |
| | | GCCAACTGGAGGAAGCCACTCACCAAAAGCTTCATTTTCCACCAGATACTTCCTATTTGATCTAGTAGAA |
| | | AAAATGTGTTTAAGCACTAAAAAAAATTAAGTCATATGTGCTCATTATAGAAAAATTAGAAAACACAGGT |
| | | AAGTCAGAAGGAAAAAAAATCATCGCTTGGATATAAACACAGATAATGTTTGGTTTGCAGCCACCCAAAC |
| | | AGATTATATTCCAAATATTGTCTTAAAATCTGATTTACTGCATAATTTACTAGGAACATGCATCCATGTC |
| | | AATAAATAGACATCTGCATCACTTTTAATATCTGTATATTATCCCATTGTTTGAATTTCTTTTTTTTTTT |
| | | TTTTTTTTTTTTTGAGACAGAGTCTCTCTCTGTCACCCAGGTTGGAGTGCAGCGGTGTGATCTCGGCTCA |
| | | CTGCAACCTCTGCCTCCCAGGTTCAATTCTTGTGCCTCAGCCCCCCCGAGTAGTGGGGATTACAGGCATG |
| | | CACCATCATGCCCGCCTAATTTTTTTGGTAGTTTTAGTACAGATGGGGTTTTACCATGTTGGCCAGGCTG |
| | | GTGTTGAACTCCTGGCCTCAAGTGATCTACCCACTTCTGCCTACCAGAGTGCTAGGATTACAAGCGTCAG |
| | | CCACTGCTCCTGGCCTAAAGTTACTTTAAATTAACTGATCTCCCATTATTCGCCACTTAGGTTTTTTAGT |
| | | TTTCACCATTATAAGCAATGCTATGATGTACATTCAAATGGAAATGTGTTTACACACTTATTAACAGTCT |
| | | TAATTAAGAAGCTCTCCATGTGCTGTGTCTCTAACATCTGCAGGTATGTACACAAATACATGCACAGCCA |
| | | GCATCCATCTTTTGCAGGGACATTAATGATCTTGGCTCTGAGCAGCACCCTGTCCTGGGAGTTCTAAAGT |
| | | CCAGAACAGATTACAGTGAGCATCTCCTGGGGGATTTAGAGACATCAAAGAAGGCTGTGTCCGTGGTTGA |
| | | TAATGGGCCTCCCAGCTGACTTGCCAGGGCTGGGCCTTAGACAGCCCTGTCCAATGATTTGTCAATGAAT |
| | | AAACTGTTCCCAAACAGGCTATGCAGTTCAGTGGGAAAGCACAGGTATGGGACACGGAGAGCCCCAGGTG |
| | | GACTACTTGACCTCTCTGAGCCTTAATTTTATCACCTGTGAATTGGGAATAACTGCTTATTTCATAATAT |
| | | TATTATGAGGATTTAATGAAATCATGTGGGCAAGGAATTATTTAGAATTAGATTCAACTCAAGTGATGAC |
| | | AACCCCAAACTAACAGCAGATAAAACAAGACACAACTTGTTTCTCACTCATCTAAAAGTCTACGTGGGTG |
| | | GTGCACGATGTTCTATTCTCTTTCTCCTCCACACTAAACAGGCCTCAGCCTCATCAGCCAATAAGGCAGG |
| | | AGCTGCCTTCCAGGCAGCGGAATGGAAGAAGGATGAAGCAAAACAGAGGGCAGAGTGTGCACATGTGCTA |
| | | TGTTTAGGGAAGGTTTTCTGAAGTTCCCACATAGTACTTCCACTTACAAACCCAACAAAAAAGGCTATGG |
| | | CTAAGGCAGCAGGGAGGAGCAAATAATGGGAGCAACTAGATTTTGCCACAGCACCTATCACAGTCTGGTT |
| | | TATAAATGGTTCTAGGCCAAGAACACCCGATCCCTGCTCTTTTTTATATTCTAAAGCATGTATCTTTATA |
| | | TTTCTCAAGCAATATTTTCTCTCTTTGAATCACAGCTCATCTGCTGCATCATAGGGATCCCAAAAGAAGG |
| | | ACCCAAGGAACTTGTCTCAGTCCTCTGTGCCCCAAGAGGAAGCTTTGCTTGTTTGCTTTGCTGTCAATGC |
| | | TGAGGGCTCCTGTGGCTGCCTCCACTCAAAACCCTCCAGCATCAGGACGTCAAGGCTGTGATACTGTACC |
| | | CTGAGCTCTTGGCCAGGGCGAGGGAGGGGAGGCCAAGCCTACCTACATGGTGTTTCATTTCCTAAACGAA |
| | | CCCTTACTTCCACGCGGTCTGTCCAGCTTAGAAACTTATTTTCAGTAGTGTTGGTCCTTGGTCCCTGGAC |
| | | AAAATGTAACAGCCAAAGTCCTAGAAAAAGGCAAGCCAGTTCCTGCCATTTTCTTTCACTTCTGCATTTC |
| | | CTCACTATTATACGTGCCTTCCATTGGAGCAAAACTGAATGCCACGCATATGCACAGGAGCTGTGCGCGC |
| | | TCTGTCTCTCTCACTCACTCTTTTTCTCTCTCTCTCTTTCTCTCTCAATCTCTCTGTCTCTATCTATCTC |
| | | TTACTCTTTATCTCTCACTCTCTCACTCTTTCTCACTCTTTCTCTCAATCTCTTTCTCATTCTCTCTCTA |
| | | TCTTTCTCTCTCTCTCTCTTTCTCACACACACACACTCACAAACCCACACTCTTATTCACATCTGCTCAC |
| | | CCTAGCCACTCAAACACAATCCCTCATTCAGCCTGGAATAAGTCCAGAGGGCGTGGGCCTGATTCAGAGA |
| | | CAATCAGTTGTTCTCATCTGGGAAATGGGGCAATGTGGTCATCTCTAGGGACCCTCCCTGCTCTAACATT |
| | | CTTTGAATGTGGTGGGTCCTGAGGTGGAAGCACTCTGTCCCTGACTTCTAGTATATGTGGAGATAGGGTT |
| | | ACACAAATATTTTATTGGGCAGAACTTTTATAAAACAATTTATCATAAGCTATCGCAGCCAGCAGCAATT |
| | | TTTCCAACCTGGATTCCACCAGGGGAGCTTGGCCGGTGTCTGAGTGCCACTTTCAGCTTGAGAAGCAGGT |
| | | GACTCAGTGAAAAGAGCAAGGAGGAGACAGAGGCAGATTCAGTTCCTAGGCCCTGGGCCACCCACCTGCA |
| | | AGTTTGCAGCCCAGTCAGTGCAAGTCAGCTAACTGTTCTGAACCTCAGTTTCTCTGTCTGTAAATTAAGC |
| | | TAAAAATTCTTCTTTCAAAGAGTGTCAGGATGAAGTGAGATCGTGTATGTAGGGCATTTAACATAGTGCC |
| | | CGACACACAGGGAGCATTCGGTAGGTGCCAGCTCTCCTCCTGGCAGGAGAGAGAGAAACAAGGTGAAAAG |
| | | AGTGAATTAAAGAAGAGGAAAGTCAAATGGGAAAACAGGGGGAGGAGATAGAAAGTGTATGAAAAGGAAA |
| | | GAATGGTGCGCAATAACGGCGGTGTAATGCCACCAAAATCCCCTCAACTACTTCTGGGCAGCACCCTTGA |
| | | CAGAGTGAATGCTTTTATGAGAATGTAAGCGGAATGTGTTCCCAGATTTGCAGTAATATTGCCACCTGGT |
| | | GGACAAACCCATGCACCTTTGAATTTTCCAAAATATTTCGATGAACTAGCTTCCAGTCCTAGATGTATTT |
| | | TGAAAGTGATTTGTAAATTGTAAGGAACTATTCAAATTCTTTCATTAATGTCACAAATCAACTGTGTCAT |
| | | CTGTATGCCACCCACTATTCTGGGTGCTGGGGACACAACAGCTCACAAATCAGGCAAAGTCCCTGCTCTC |
| | | ACCAAAATGATATCCTACGGGGGATTACAGATACAAATACGTAAACAGATCCATCGGGAGGAAACTCTCA |
| | | GATGGAAATGAGAGCTATGAAGATAACACAACAGTACATGACAATACAGAGTGACTGGAACCAGGAACAT |
| | | TTCTCCGAGGAATAAAATTTGAAGCGAGCCATGAGAGGGTCTACAGGTAGAGTTCCCAGGCAGAGTGAAC |
| | | AGCCAAGCACAAAGCTGCACCAGGAGAGAGAGGTGCTCGCCGAGAGACAGGGAGGGGAGTGTGGCAGGTG |
| | | AGCTCAGAGAGGGGCAGGGCCACACACATCGGCCACATGGGCCTTGGTAGTGAGTCGAGATTTGATCCCA |
| | | GGGTTTATTGGAGTGGATAAGTAAGCAAGGTGACTGAGGTGCTCGGGTTTACATTTTTATAGTTCAAGCT |
| | | GGCTGCTGGGTGGAAAACGGAAGTTGGCAGACCAAGGACAGAATCAGGCAGACCCATGTGGAAGTTTCTC |
| | | TAGTGGTCTAGGTGGTGGCTTGGGTAGCGTGGCAGTATTGGAGCTGGAGAAACGCAGATGGATTGGAGAT |
| | | TTGTTTTGGAGTGACGCCATTCTGTCTTGTCAATGGATTGGCGAAAAAAGAGGCATCAAAGATGAGTTAC |
| | | ACATCATTGAAGTGAGAACTAGGGAGATGCCAGTACTTTATTTAGTATTTTCTCAGCAGCTCAATCCATA |
| | | AATAATTTTTGGAAGACAACAAGCAGTTTCACAAACTACTTATAAGTCCTCAAGTTCCAAGGTAATTAAC |
| | | GTGGGTGTCTCATTGCCTCAGAGAACACAGCGCAGCACGGAAATTCTACAAGACCTGACGGACAGGAACA |
| | | TCTCCGACTTCTTGGTAAAAACGTATCCTGCTCTTATAAGAAGCAGGTAAGAAGAAATCCTTTTATGCTT |
| | | TTTATCCTGGCTCCCTGTAGAAGATATTAACTAGGGACAGAAGATAATTTTCTCTCTCAATTTATGTATG |
| | | ATCAGGGCAGTAGATTTTTTTCTTTTTTATCTGATTTGAGGGCCCCATTCAACATAAAAAGCAATTGAGG |
| | | CACATACAAGTAAAATGTAACTTAAGATTAATTCTTTTTTTGTTGTTTGTTTGTTTGTTTTTACATTTAG |
| | | GGCAAGCAGTCTTAAATTTTAACCCACGTATTATTAAAAGTTATATCAGAAGACCATAGAAGTTATTCAA |
| | | AAATGCAGCCACATATTTTAACTAGTTAAAAGAGAGAGTAAAAATTTGGAGGGAGGTGGAGGAGTATAGG |
| | | GGAAAAGGTAGAAGAAAAAGAGAAAATAAGTAAGTGGCAAAAAAGAGAAAGGAAAAAGATAGGGTGGGAA |
| | | AGAGGCAGCGGGACAGTGTCTGAGTCCAGCACACGCCAGGGCGAGCCAGGTCAACTGCAGCTGTCATATT |
| | | CTAACTGTGAATTATCATCTTTGATCACTGCCCTTTGAGATGCCAATGAACTTTTCAAGAAATATCTAGT |
| | | TCTCTTGGCTCTCCAGCTGTTCTTATCAGCCCCATCCAGGATGGAACAGCTTTGGCAGCCCGTATCAGAA |
| | | CAAGCAGCTTGACAGGGGCATGCCATGCCAGGAGAGAGGATCCTAAGGAAGCGTGGTCCAGTCCGCACAG |
| | | GCTCTGGGGCTTTAAGATAAAACCTCCTGTCTAACTTTAGTAGGACTTTCTGTTGCTTCACCTGCCAGAG |
| | | CCCTGAACGAGGGATAAATTGACTTAATTAACTAGAACACACTGCAAATGGTGAAAGCATTTAGCAAAAC |
| | | AAAGAATGCCATCCAAGCCCCAAAATAAAAGCAGAATAAATAGAATGCAATAAACAGCAACCATCCCAAA |
| | | CTGAGTTCTCAGCAGCAAATCTCCAGTATGAAATTTTGGATTTTGTGCGTGTGTGCTTAAAGGTGGATGA |
| | | CAATGACAGTTCATGGGATTGAGCTCTGGGGTCCAGAGTTGGCATCTGTTCATTTCCCATTTTGTCATTT |
| | | TACCCTTGATTGACTGAATGTCAGTGCCTTAACTTTGGGCTGTGGAGTGAGTCGGAACTCCCCCGAGGTG |
| | | TGCAGGTGGTTGTTAGAGTCTCATTTTTGCAGGGTGGAAGACAGGAGGGCTGCAGCCTTCATTCCACACT |
| | | GACATGGTCATTGCCGTGTGTTCTGGGTCCAGATCAGGCATATTGACCTGACATATGACCTGACAACAGG |
| | | ACCACTCAGAAAGTCCAGCATGCGGGATATGATTTGGAGAGCCAGTGGGGGAAATCATAGGTCCTTTCTC |
| | | TGCATGTGTATTCAGGCAATGTCCCAGGGCTGGGCGGCTTCCGCATTGCTTGGATATCGGAAAATGCAAA |
| | | AATGCCCCTGAAGACTGAGACTTCAGTCTTCAAAATGAATGTTTGGGAAAGAAAGTTAACGGCACTGCTG |
| | | TACTTGTGGTATTCATTGCATTATTTTATTTTGGCTTTCAGCTTAAAGAGCAAATTCTGGGTCAATGAAC |
| | | AGAGGTAAGAAACTATTTTTATCAGAATTAAAATCTCAGATTGATTCATTGTTGAAATAATTGCACACTT |
| | | TTAAAAGGCACACCTCACAGCCATGAGGAGGGGCTGTTCTGTAGGTGCTCAGGAAGTCACAAGACACGTC |
| | | CTGAAGAATATGTGGCTAGGGACATCCCAGACTCAGAAGACACTCAGTGGTGCCTCTTCTTGGAGGACAT |
| | | AAGTGGGGGTGGCATTCCCTGATGTGGCGTTTCAGAGCATTCTCACCCAAAAAAAGCTTCTAAAACCTCC |
| | | AAGTATATAACAGTTTATAATACTCCAACAAGAGGGCCTTGTAGCCTAAACCCGGGACACTCCTTGGCCC |
| | | ATTCCTTTTAAGCTTCAGGGAGTGTGGGCCAGCCCCAGACTCACCCCATTCCTGAGGCATCCTGGAGGTT |
| | | GAAATATTTCCAGAGGTTTAGAACCTCACCAAGTGGGACTCTAGGAGCCTGCTGCCTCCCAGCCTCCCTC |
| | | AGGAACTGCACCTCCAGAACAGGTGCGGGGCTGACATGTATGTGCTTTCCTGGGCAGATTCTAGACCGTA |
| | | CACATGAAATCTGGCTTTCAGGATTGCTCTCCAGAGGGACCTGTGGGGCCTCGGCTGAGACAGAGAGTAG |
| | | GAGTGAGGCAGTGATTCAAGGCCCTGAGAAAGAGCTCCTCCTCTGCTTGGTATAACCAGCTAATTCATTC |
| | | TGTTCTGTTGACTTTGGCTTCTGCCCTGCCTTTGAAGGGTTTGAGGCCAGGGAGTGATGCACTCAGACTG |
| | | GTGTTTCCACACAGTCACTTCAGACTTCCAGGGCAGTACAGGAGATAGATCCCAGGGCCAGTGAAGAAGC |
| | | AGAGCACAAGTCCAGGCAGGAGAGGCTAAGGGCCTCCCTGAACAGGTGTGAGGCACAGAAGCCCCGAGAG |
| | | GTAGGGATGACAGGATGAAGATGGGTCCTGTGCTGCTAGAAGTACCTGCAAAGCACAGAGGTGGCACAGA |
| | | AAAGGAGTCCTTGGCTGGGATGGGAGGAGATGACATGTGACATGTGAAAGAGGACCTGGAGTTGGCTCGA |
| | | TGCTCCCAAAAGGGAAAGGTGCCGAGGGGAGCTAGCAGCCATGCAAAGGCAGAGACATGCAGGCAGTCTG |
| | | GGCCATGAGGAGCTCTGGAAGTGACTCGATATGTCCAGAATAGGCCACTCCAGGGAAGGGCTGAGGAAGG |
| | | ATGAAGTTGGAGAGGGGCACAGACCAGATGCAGAAGGGCCTCAGAGGCCAGGATGAGGGTTTGGACTCCT |
| | | TCCTGGAGGCAGCAGCAGTGGGAAAAGAGTTAAAAGCTGGTTTGTAAAGTGGAGCCATGTTGCTCGCTGG |
| | | TCCAGGCAATTCCCCCGAAAGTTCATGTTTCCCTACAAAACCCGAGAGAGCTACTAGTAGGCGTGAAGTT |
| | | CGTGGCCCTGGTCTGAGGATTTCCTGTTTCCTTGTCAGGTATGGAGGAATTTCCATTGGAGGAAAGCTCC |
| | | CAGTCGTCCCCATCACGGGGGAAGCACTTGTTGGGTTTTTAAGCGACCTTGGCCGGATCATGAATGTGAG |
| | | CGGGGTATGTAAACAGACTGGAGATTTGAGTAGGATTTTTGACTTGCTTAACTACCATGAATGAGAAACT |
| | | CTCATGAGTGATAACAGGAAAAAAAAATTAAAACCGTCTTGTTTGTTTGTTTACATGGTTTTTAGGGCCC |
| | | TATCACTAGAGAGGCCTCTAAAGAAATACCTGATTTCCTTAAACATCTAGAAACTGAAGACAACATTAAG |
| | | GTACTTGACCTATGTATAATCTGCTCTGGAGCTAAAAATTTACCTGAGCTGGTTATTTTATTTTTACTTT |
| | | CCTACCTTCATTAAATTCCATCCCTCCTCCTGCTGAAATCTAGCAAGGAATGTCTTCCAGCTACCAAACC |
| | | CTTCCTGCTTCTCAAATTTCCTTTCCTTCACTGATTTCTGCTTTAACTAGCTGTTAGTGCAGCGTCTCAG |
| | | ATGTCCTCTCCACCCTCTAGGTGTGGTTTAATAACAAAGGCTGGCATGCCCTGGTCAGCTTTCTCAATGT |
| | | GGCCCACAACGCCATCTTACGGGCCAGCCTGCCTAAGGACAGGAGCCCCGAGGAGTATGGAATCACCGTC |
| | | ATTAGCCAACCCCTGAACCTGACCAAGGAGCAGCTCTCAGAGATTACAGTGTAAGCCACCACAGCCCCAG |
| | | CCTCACCACTTTCTTGTCACCTTCTCCACTCTTTGAACATCCTGAGAGGATTCTCACCACCGCGAAGTGC |
| | | TGATTTGGATGGTAATGCTGTTTAGTCAGGCACATATGAACATCCGACTTTCAAATAAGTGCCTCACACT |
| | | TCACATACCAGACCTCTTGGTCATTCTTTCTCCCCAACATTTATGTGGCAAGTAAGTTTACATTTGGTTC |
| | | CATTCCCTTTTGGCTTTTGATAGCAAGTTGCTCCTGGAGCTTATACAATTATTATCTTTGCTATGTGCAA |
| | | AGCAGCTGCCAGGAACTGGCAAAGTTCAGTAAACCTTTCAGCTCCCTCGGAGTAATTATCTTAGATTCCA |
| | | GGAATTTCCTCAGAAGAGCATACTTTGGAGATGTCGACAGAGCTTTGCTACCCTCAAGCTGAGGCTCTTC |
| | | TTGCACAGTTTCAGCCAGTGGAGACAGTGGCCTTGTGCGTTTTGTAGTATGTTCACTCTATTTGAGGCCT |
| | | ACATGGAGGAGGGGTTGGTAGGAGCACCTTTGTTAGTGCAAACTTCAGCAACGTTGTGGGGTCCTGATTT |
| | | TACTATCCTAGCACACGCTGAGTGCCAGTGAACATGCCCAGGGTCATCCACTAAAACCTGGGCCTTGGCT |
| | | CCTTGGTGTCTTCCTCTGGACACCCTAGGGCCCTAGACTGTCCTCTGTTAATTCTCACTCAGCCACACTT |
| | | TCGTGTGTCTCCTTCCAGTCATTTGTTCTAAGCTTACTACGTGTATGGATGATATGATCTGTAGTTTTAT |
| | | CAAGGTAGTGACTACCACATAGGATACCTTTGTGGAAATTAGTAAAAATGCTCTTTTCTGCAGGTGGACA |
| | | CTGTCCCATGCCAGGGGTTATGGCTTGTACATAAAGTTCAGGCTGGCTTTAGCCCCAACTTACCCCTCAG |
| | | CCAGATGCCTTCTATTTGTCCGAGGAAAGAATAAATAGAGCCAAGTCCCTGTACAACTTGCCTGCCCTCT |
| | | TTTCACTTAAATTTACATCATGAACATTTCCTTGTGTTACGATGTACTTCTTGAAAATGTGATTTAACAA |
| | | GATGATTATTAACAAAAGATAAATCTCACAGACCGTATGTCTGTCAACATAGAAAATTCAAGAGACTCTA |
| | | TAGACAGATTATTAGAGCTAATGAGAGCATTGCAGTACATAAGATTAATATAAACATCTATTTCTATACA |
| | | CCATAAAAATAATTAGAGAATATAATAAAAAGAAAGGTTGTCTAGAAATATTCACATGAAATAGAAAGGC |
| | | AACCCGCAAATACCCATTTAACCTTGGTCCATATGGATTAAGACAGTTTAGTGGAGTGACAGCTTCAAGG |
| | | TAGAGAAGAGGAACCTGGAGGCCACACCTGGGCGGGTGTAAGGCCTTCCCAAAGCCTGACTTTGTATCTT |
| | | CTCCTCCTTCTGCTCTTCCCTCTTCATCGCCCTCTCCCTGTGTCTCTGGCCCTGCTGCAGGCTGACCACT |
| | | TCAGTGGATGCTGTGGTTGCCATCTGCGTGATTTTCTCCATGTCCTTCGTCCCAGCCAGCTTTGTCCTTT |
| | | ATTTGATCCAGGAGCGGGTGAACAAATCCAAGCACCTCCAGTTTATCAGTGGAGTGAGCCCCACCACCTA |
| | | CTGGGTGACCAACTTCCTCTGGGACATCGTAAGTGTCAGTTTACAGCGCCTCCCTCCCCTCCGTGGGCCC |
| | | AAGGTGGAGCTTGTGTGTGCTCTGAAGGACCAGACCAAGAGGGGAGGGGTTCTCACGGTGCCAGGGCTGC |
| | | TGAAAGGCACTGGGCCAAGGGCCTTGTGTATCTGCTGTCCCTTGACATCTTCTCAGAAAGGCACAGAACT |
| | | AGGAGCCCGAAGCTAGGAAAGGCTGTGGGGTGCAGCTTAACAACTGGTGAACGGGGGCTCTCTATGTCCT |
| | | GCACTGAGGGGTCTTCTGACCCATCAAATAATCACTGCACCGCAGGCATGAGTCTGGCCTTCCTGGCATC |
| | | AGTCTGGCGCTGAGAAGGTAATATGAAGGGGTCTTTCACCCCAAGTCCCCTTCTCAAATCCTGCCCCACC |
| | | TTCAAAAGGGTAAAGGTAAAACTTTCCCTGTGGTAGGGTCACCAGATAAATACAGGACACCCAGTTAAAT |
| | | TTAATTTCAGATGATGAATAATTTTTAGTATAAGCATATGCTACTTCAAATATTGCACAGGACATATCTA |
| | | CACTAAAAAAAAAAAAAAAAAAAAAAAAAACCTGGTTGTTTATCTGAAACTCAAATTTCACTAGGCATCC |
| | | TAGATTTTTATTTGCCAAATCTGGCAACCCCAGCCAGTGGCCAAAATAATAAGACCTTCACTTATTAGAT |
| | | TAACCACCGCTACAGGGAAAAATGAAGAAAAAATATTTATTAAATCAATAGCACACTACCACCTTCCTGA |
| | | CAACCAAGGTTGGTGGGGGTAGGGAGGGGTCAGGATAGCGTACCCTATTACAGGCTGCAGGGTCAAAGGA |
| | | ATTGGTAGTAAAGGCCTAGTTATAATGTAACAGGGATCATTATGACATCAACCCCAATTTATTCTAGGTG |
| | | TCTTGAGTAGTAAAATCTCAACATTTTAAGACCAACATGAGCCTCCATTTCATGTGATGATAAGATATAC |
| | | CAACTGATGGAGACCAACACAAATGACCTTCTCATCCATGGTTTTTTAAAATGATGGTGAATATTGGAAT |
| | | TCCTGAAGATATGATTTCTATCTTACTCAGCTTAGTAAGCAGCTATCACTTAACAATACAAAACCAGAGA |
| | | TTATCAGTAGCAACTAAATTATTTCCTCTCTCTTCTGTCTACACGAGGAAACACTCATAAATGCACGGGG |
| | | AGGAGGTCAGAACCTGAAAGCCTTTCTTTGGATAAGAGCATCAACTGCAGGTACCACATTGGCCCTGTGA |
| | | TGCTAATATAAAAGGAGCTAGGCCCACCGGTACCGAAAAGTTACTTAGAAAAGTGCGGAGGCTTTTAATT |
| | | TTACTTTTTTTAAAAGATAAGAAATAGAATTTACACACTTGGGGCTGGCCCACGTGTTTCTGTGTGTGTG |
| | | TATGTGTGCACGCACGCGCGTGTGCGCTTACAGGGATCTCTGAGCCTATGGAGAGAGATGTAGCTAGGAT |
| | | AGAGTGGACATCTGAGGTGGGAGGTGATACTAGCTGGCAGTCCAATGAAGGGGTAGAAGATGGTAGGCAT |
| | | CATGTTAGCAGGCTTTCTGATGCTCCAGAATTTTAAAGCTGGCCTGGAATCTCACCTCCGCGATCCATCA |
| | | TTTTGGAACTTAGGACCACCATTAGCCAGTGGCAAAAAAAAAGTTGAATGAAGGAACAAACAATTATTGC |
| | | TTATGTAATTCACTTAGCACATATATGATGTTTTAAATTCTTATATGTGTCATCTATTTTTCTTTACTTT |
| | | AAAATTTTGCAACAGTTACAGACTTATGGAAAAGTCACAAGTACAGTTGAAACCTTTTTTTCTTAGTCAT |
| | | TTGAAAGTAACTTCTCAGCAAGATGCCCCTTCTCATTTATTTCTCTCTTCCTGTCTCTCTCTCTCACACC |
| | | CCTCAGCACGTCCGATGTATACTTCCTACAAACGAGGATACACCCCATACAACCACAACACAAACTGTCA |
| | | ACATGAGGAAACCAGCACTGATGTGTCATCACCACCTAATCCTCACACCCCACTCCTCTTTCGCCCATTG |
| | | CCCCAGTGATGTCTTTCAGAAAAAAGGATCTAGCTCAGAATCATGCATGACATTTGATTGTGCTGTTTCT |
| | | TTAGTCTCGTTCAGCCTGGAAGAGTTCCACAGTCTTTTGTTAACACTCATGGTCTTGACACTTTGAGGAC |
| | | TGCAGGCTGGTTATTTTGCAGAATGTCCCTTGGTCTGAGCTTGTCTGAGGTTTCCTCTTGCCCAGGTTGA |
| | | GGGTGTGCATCTTGGCAGCAGTATCAGCAAACAGATGCTGTGTTCTCACTGCATCCTATCAGGTGGCTTC |
| | | TGATTTCAATTTGCTCTGTTACTGATGATGTTCAATTCGGTCACTTAAGAAGGTGTCTGCTGAGCTTCTT |
| | | CACTGTAAAATTACTCTTTTCCCCTTTATAATAAATACAAATTTCAGGTAGAGGCACTTCAAAGATATAT |
| | | AAATATCCTATTCATTATACAATTTTCCATTTATTCATCCATTTATTTATCTCTGTATGCAGTCATGGTT |
| | | CATGTGTTAATCAATGGACTATGATCCAAGACTATCATTATTTATTTTGATATTCACATTATCCCCACTG |
| | | TGGTCAGTGGGGGGCCGTTGAAGCTGGCTTCTGTATCGTCTTGACTTGGGTCCTCATGCCCCTGGACCTC |
| | | CTCCATGCTCAATGGCACAGCAAGATATTCCAGGCTCATCCTTCCATTATCCCCATTCCTACCCTCTCCC |
| | | CAAGAAGCCCTGGTTCCTGCCAGTGGGAAGTGGCCCTCAGAAGCCAAGGTCTGAGTGCTAGATATGTTCA |
| | | TTGCCTCTGGAGCACCATTGGTCCCAGGCCTTCTCAGTGATAGAACTAGGGAAGATATGGATGTACACAC |
| | | ACAGGTATGCACACACCTCTATCTATAGTTCTCTATCTACCTATACAGTGAACACTATGAGCTCTCCAAA |
| | | ACCAACTCCACAGGGCTCATTCTAGTTTTTTTTCTTTCCACATCTGTAACTCCCTTCTCCAACAGTGAGA |
| | | CGCTGGCTTCTCTCACTCCCAACTCATTTATCTACCGGACCTATACACCTGAACAGTGCCCAACTCTGCC |
| | | ACCATCCCCTCCCCATGTGGATGCCGTCCTCTCCCTGCTCCAGCTGCCTCTGCTGCATGCAGGTCCTCCT |
| | | CGTTCTGCTCTGGCTCTGATACCCTGCACCAGATCAGCCTCCTGTAAGGATATCTTTCTCATCCCGTTGA |
| | | GGCCTCCACACCCCACGGCAGGTTGCCCCCTGAGGAAGCCCGTCTCTGGTTCTTGCCCTGCTCCTGATCA |
| | | CCATGGCTCCTCCCCTAACCCCACTGTTGCCGTCCCCTTTCTGTGCCCAGTATAGTGGCTGTAGGACTAA |
| | | ATTGTTTAAAAAGGGTATCATTATTTATTTGAGCTTTGTGAAGCCAAGAACTAGGCTTTAAGTTTTTCTG |
| | | AATTCTGAAGACATGCTTAGAAAGAAGAATCAACAAAACTTTATGACCAAATAGAAAGAGTGAGAGACCA |
| | | GGCAGAATTTTGTAATTGATCCTTTCAAAAGATACAAACTAAAGGTTCCCTTGGCAGGGAGGTAGGGCAT |
| | | GGGGTGGGGTAGGAGGACTAGTGACAGCTTAACATATGTTTGCCAACCAAGAACTGTTTAAAAAGCAAGT |
| | | CGAATCAGAATCCCAGACCCTACGAGCTGGAGGAGCCTGGCCCCACCCCTCATTTTGCAGAGCTGGCAGC |
| | | AGGTCTGAGAGGTTAAGTGACTTGCTCTCCTCTTCTCTTTCCGAGATGAATTATTCCGTGAGTGCTGGGC |
| | | TGGTGGTGGGCATCTTCATCGGGTTTCAGAAGAAAGCCTACACTTCTCCAGAAAACCTTCCTGCCCTTGT |
| | | GGCACTGCTCCTGCTGTATGGGTAAGCCGTTTGGGCCATTAGCTAATGCCTCTGAAGAGAAGCCTGGTGG |
| | | TGGGGGTGGGGGATCATCTCCTGACAGAAAACCTGGGCTGTCCTGTGGTGGTAGCACCCACAAGTTTAGC |
| | | TTCCGGCCCCAGGTAGGGTCTGAAGCTGATAACCAGGGATCTGTCTGGCTTCTGATTCTGACTCCACTGA |
| | | CAGAGGTATCTCTGAGGCCTGGTCCTGTCAGTGACAATGAGAGAAGTCCCACATGATCTGAATCTCCTAC |
| | | TCAAACTGAGGCCTTGACCAAAGCCTGGGGGCAGCCATTCCCCAACCCCTCACCCAGCTCTGACTCTCAC |
| | | TCATCTGTGGCCAATCTGTCCACCTCAGTGTCCCCATGTGAACTGGCCAAGAGTTACCGCCCACAGTAGA |
| | | AGACTCCGGCCAAAAAGCTCCTCCTGAGTCAGGGACAGAGGATGACACAGGGGTTACATCAGCAGAGTTA |
| | | CAGGGCCCAGCATGCAACTTTCTTTCCCACGTGTGTAAATTTGAATGAGTAATTCATCCATCTCGGCCTC |
| | | AGTTTCCTCATCTGTAAAAGAAAATAGTGATCCTGGTCCTTCCTCTGTGGGCCAGTAGAGCCTTGCCAAA |
| | | GCATTGTTCTCCACATCTTTCTCTTGGAAATAGAGAATTTGGGAACCAACCTGACTATAAGCTGTGAAGA |
| | | TGAGCTCACTGGGCTCATCTGAGATGACCTCAGCTGGGCTTTGCTGACCCAGGCTAGAGTGGGAGGTGTT |
| | | GCAGGCTGGAGAACCCTCCTATGAATTGTACAGGGCTTTGTAGTTTACAGAGTATATACACAGCTAGCAG |
| | | CCCATTTGCTCCTCACAAAACCCCATGAAGTGGTCAAGGCAGGCATCATTATCTCCATTTAAAGTTGAGG |
| | | CACAGAGACCAACAAATGGAGTATCTCTCTGGTCCCCTGGGACTCTGGCCAGTTCACACACATCACCTCA |
| | | GGTGTAAGGGGAGTGCATTATATCCAGACGTATTGTAGGTGGAATGGAATGTGGAACTCCATCACTCTGA |
| | | GTTGTCTCATTTCACACAGATGGGCGGTCATTCCCATGATGTACCCAGCATCCTTCCTGTTTGATGTCCC |
| | | CAGCACAGCCTATGTGGCTTTATCTTGTGCTAATCTGTTCATCGGCATCAACAGCAGTGCTATTACCTTC |
| | | ATCTTGGAATTATTTGAGAATAACCGGGTGAGCATAACTTTCTTGGCTTTTTTGTTTGATTAGTAGGATA |
| | | GTAGAGTATGTGTTGGTCGAGCAGAGCCAGGGGCAAGCATCGTACATGTAGCAGCTGTATGCGGATGAGT |
| | | GCCACTTTCTTCCTCCCTACCCCCGACCCTGCCTCCTTTCCTTCCTTCCTTCCTCCCATCCTTCCTTCCT |
| | | CTTTCCTTCTTCTCCTCCCTCCTCCCTCCTTCCCCCGTCCCTCCTTCCTTCCTTTTTCATTGCTTCCTTC |
| | | CTTCCTTCGTCCCTCCTTCCCTTCCTCTTTCCTTCTGCCCTCTCTCCCTTTTTCCTTTCATCCTCCCTCC |
| | | ATCCCTCCCTCCATCCTTCCTTCTTTCTTCCTTCTTTCCTTCCTATAAGCACCTTTTTCATTTCTGTGCT |
| | | CTGAATGAAATGGTTTTCTGTGTTTATTCTGCAAGCAAAACTTGATTCTTGCAATAAACTTTAAGCTTTG |
| | | CTTACTCTTTCAGAAAGGTTTTCTCAGGGACTTTGGGTGTTGGGTTTTACACACACACACATCAATACAT |
| | | TTGGGTAATTTCAAAATCTAAAAGGAACAAAAAGGCATACAATGAAAAAATCTCCTTCCTACCCCTGTTT |
| | | CCCACTCATGCAGTTCTCTTCTCCAGAGGCAAACTCTTACTTGAGTTTCCTGTGTGCTCTGGAGACACAT |
| | | CAGCAGATCCCTATACGGTCTTTCTCCCGCTTTCTTATGGAAATTGTAACACTCTGACATATACTATTCC |
| | | TTGGGCAAGTTAATCTTGATGAAGAGACTGGGTGTTCTCCATGCTGAATGCCTCACTTTTATGAGCTGCC |
| | | AAGCCCAGTTGTCCCTTCCACCTGACCTCCCCCTGTCCAGAGACAGATGGCCAAACTGAATCATAAAAAG |
| | | AGGGGGAAAAAAAGAAGGCAGTCGCTGCAGGGCTGTCTTTACTCCACACTCCACACTCCCAGTCCCCACC |
| | | GCTGTGTCTGAGTCCTGGCTGTGGCTGTCCTTGGAACATTTGCCTCACCACGTGCCTGTGTCCCCAGGCG |
| | | CCTCAACCTTTCCTCTCCTCATTAGCTCTTCCCAGTTCAGAGGGTGGGACCGGCCAGCACATCTGCACTG |
| | | CTGCCCTGCCACACCCACCTCCACCTGCCTCTGGGCCCCACTGGGGAACACAGGACAAATCTGTGCGGAG |
| | | GCCCCACCATGAACCGCCCAGACCCGTGGACCCCTGAGACTGACTCTTTCCAGATCTTGTTAGGGTTTCG |
| | | TGGCTGCTAGGCAAGTAACGAAGCCTCATCTGTCCCATGAATGATAAGAAATTCAGCATGTCAGAGTCAG |
| | | ACTCTGGAAAGGCGGGGGGATAAGAACACAGCCCCAGCAGATGGCCAGAGCACCCAGGTGACTGAAAGTG |
| | | CTGCTTTGCAGAGCTGTGTTTGCCACAGGCTCACAGCCCACTAAGTCTTAAGACAGTTTTCCTTCAGAAT |
| | | AATTAAATAGCCAGCTTAAAGCAACTCAGAACATTTTCCCCTCTGAGGCTGCACCCATTTAGCCAACATT |
| | | TGCTAAGCACCCGCCTTCAAAAACCTGGTATTTTCATGTAAATTATCCGATACACAGCTGCTATGGAAAC |
| | | CCCCAGTATCCCACAGGAAGCTCCCCAGCTCCCAGCAGCTGCCGGCCCGTGTGAGATCAGGAGGTCTTTA |
| | | CCAGCTGAACACCACGTGCCGGGTGTGTGCTGATATAAACAAGCGTGGCCCACTCGTCCTGCCCTCCAGA |
| | | GGCTCCCGTTCCAGTCGGAAAAGGACCTGCCCACGAAGTTTGCAACGATATAAGCCACAGTGTATGATCC |
| | | TCCATAATACAGCGTGTGACAGAGCAGCAGAGGAGCGAGGCAGATAACATGCTGCAGGCCAGAGGCAGCG |
| | | GGAAGAGCCAGGCTGCAGGGGCTGGGGGAGCCGTGGTGGAGGAAGTTCAATTTCAGCCTGTAGATTTCTA |
| | | TTAGCCCATTTAATAAATAATGAAGTGCCTACTCTGAGCTAATCATTGTGCAGGTATTTAGGAAGGACAA |
| | | AAAAATAATTAGGACTCAGTGCCCACCCTCCAGGGGCCCACTGACTAGTAGAGAAAGTAGGCAGATTTTT |
| | | AAAAAATTAATCATGGGAATGTGATAAGTGCTGGGAGAGAGGAATGGATACTTTCTCATGGGAATCTTGG |
| | | AAGGCTTGTAAGGGAAGGCACTCTCTGAGCCAGCTGTCTAAAGAAGAACAGGAATCTTTAAGAAAGCAGA |
| | | AGGGAAAAGAGCATTCTTTCCTGCTTGGAGCAATAGGTAACAGCCTGCACATGCCCAGGCCTAGAGGCCA |
| | | AAGAGCACAGTGATTCCAGAAAGAGTGGGGAGAAAGGGTAGGCAGGGAAGGATGAGGTAATGTGGGCGCA |
| | | GGTGTGGAGGCTGGAGAGGGAGGAGGTTGTGGGACTGGGAGGAGCCAGATGGAATGGACAGCAGTGGCCC |
| | | AGCCAGGAGCTATGCTGGCCTCGTACGCCTCGATGTCCCTTCTATTTTCTCAGGGGAGGCTCTGCCCAAC |
| | | ATGCCAAGTCCGACCACTTGAAAACAAGTCCCTGGCTTAACACAGACCCCAGAGAGAGTCTCCAACCCTC |
| | | CTCTCCCTAGACAATGGTAGTTGCCCTGTGAGGGGCTGAAAAGCAGAGCTGGAGATGGCTCAGGGCCTGG |
| | | TGTTAACAAATGCCTTGAGGGCTCCTGTTGTTTCAAAGTGAGTCTGCAGGGAGAGCTCCCTAAGTGGACA |
| | | GCAGGAGGGCTGCAGCTTCTCTGCACATTCCTGCTGTCACCCCCAGAGTCACCTAGGGGAGGGGTAAGGA |
| | | CAGTAATGCAGGTTCCTCACAGTTAGCCTCGGTGCCCACATGGTACTGAGCATAGTAAATGTTTAGAAGA |
| | | TGCTGCCTGGCTAGACAAAGGGGAAGCTCCCGCCCACTAGAAACTTGCAGGGAGCCCCAGTCCTTGATTG |
| | | GTCATTTAATTGATTAGCTCCTTGGCCTGGCCTTGAGGCACTGCTTGTAAGTACTTCATGACCTCCATTG |
| | | CAAACCCATGATGCTCTGCTGGACAAATCCCTCCAGTGGCCAGTCTGGCTGCAAGGACTCTCTGTCTGCA |
| | | GGCCTTGCCCTGTGCTGTCCTGTGAGAGCATCTGGGCCCCACCTGCTGAAGAGAGGGGGGGTGGGGTTTG |
| | | CCCCGTTTCCAACAGTCCTACTTCTCTGTTTCAGACGCTGCTCAGGTTCAACGCCGTGCTGAGGAAGCTG |
| | | CTCATTGTCTTCCCCCACTTCTGCCTGGGCCGGGGCCTCATTGACCTTGCACTGAGCCAGGCTGTGACAG |
| | | ATGTCTATGCCCGGTTTGGTGGGTGGTAGCCGAGGCCCATGGAGCATGGGCCCTGGGTCCAAAGCTGGGA |
| | | GGGTTACCGGGGGGGCTCCTGCATCAGACTGTGGCAGGGGCTGGTGCTAGGAGGGGACCTTGTTGGGCTG |
| | | GAGGTGTCCTGCCAGCTGGAGAGGATTAGGGTGCCTCTGTTTCCATGGCTGGGGAGCCACAGGAGGGATG |
| | | GAGGGCAGCCCTTATGAGGCGGGTGTTTGGCTCTTGCTCAGTTCCCACATAAGGCCTGGTCTAGTGGGCC |
| | | CTGTGCTGTGGCCAGGTCTGTGGGGTGAGCTGGGGCGGCTGAAGTGGACTCAATTCCTGTTGATGCCCAG |
| | | GTGAGGAGCACTCTGCAAATCCGTTCCACTGGGACCTGATTGGGAAGAACCTGTTTGCCATGGTGGTGGA |
| | | AGGGGTGGTGTACTTCCTCCTGACCCTGCTGGTCCAGCGCCACTTCTTCCTCTCCCAATGGTACGTCCAT |
| | | GCCACACCCTGGGCCAGTGGGCAGCTCAGGGCATCCAGAACTGGACCTTATACCCACATGGTCATTTCTT |
| | | TCCTCAGGAGCCCCACTCCACAATGTTTTTTCTACATTCTCAAAGCCTGGCTTTTCTCCAATAATACAAG |
| | | TAGAGGATCGGGTTAAAATAGGCACATTCAAATATGTGAAGAGCATCCACTTTAAAATATTTAAAATGCA |
| | | GTGCTATTAATTTCAATTGCTGATATTTAATCCTTCTCATTTAATTACCAAATGTGTATTTTGATTAGAT |
| | | GATAGTATTGCAAATAACAATGGTTACAGGGTATCCAAAGTACTAGGAAATAGACTAATGTATTTATGAG |
| | | AGAAAGGACACAGCAGGCCCCTTTGCTAATTAGAGATTTGGGAGCATGGGAGTAATATGGGAGCCATGTG |
| | | GAGGGGTGCGGGCAGTGATCACGACCCCCCACTCCTGGAGGAAGGTGGGTAGCTGCCAACCCTGACTTTT |
| | | GACCAGGGCTTCTCAAATGCCAGGTTAGCTGGCAATTGCCATTCTTCCGCAGGCTCTTCCTGAAGCTGGG |
| | | TGGGCCCCTGCCTCACTCCCCTCTGCAATCCAGTCCTACCTTTATTGTCCTCACCCAGGGGCCTGAATTG |
| | | CCAAGCAGCAGCCCTTCCTAGCAAGCTTTCCCCAATAGTGTTTTGTTTCTTAACTTTTCCTCCTCTCAGG |
| | | CTGAGTGTGGTCACCTGTAAATAGATTCCAAGGACTTGGTTTTATGTTTTGATCCACAGGGAATTGATTT |
| | | ATTGGAAATGAATCTGCCTTTCTACTCACAGGACTGTGAGAGGTGAATGAGATCACAGGTGTCAACACAC |
| | | GCCTGATGAAACAGGATACACAAGCAGTTCTAGTTATGGGAGACAGTGTCAGGAATTGTTGTCCTTGGCA |
| | | CCCTCAGCCCCTGCAGACCCTTTCTGCAGCCTTGGCCATACCTTTTAGAGGCTTTTGTGTGGGAGAGAGC |
| | | AGGTCAGGAGGTTGACTACCCAAATTGACTCATTAGCTTCAAACTCTGATGTCAACACATTTGAATGAGT |
| | | CCTGCCTGCTTTAGGGCCTAAAGAGGACCAGAGAAGTACACCATAGTCCCTGGCTTCCAGAAGGTCAGGG |
| | | AGGGTTTCAAAGAAGAGGCTGTGTCTTTAAGAATGGGGAAGATTCCATTTGGTGGGGCAGGAGGAGGAGA |
| | | ACATTGAGGGACTGGAAACACATGCGGAGGCTGGGAGACGGGAATGACCAATAGGACTGGGAACCAGGGG |
| | | GAGATGCCAATTGCTGACAGAGGAGTTAGTGCAAGAGGTAAGTGAGAAGGGTAGGTGGGGCTGGATTGCA |
| | | GGGCTGTAACTACAGCTGCAGAGGGAGGGCTTCAACCTACAGCTGATGGGGAACAACAGAAGGTTTTGAG |
| | | GCATGAGGTGGCCTGATGACAACTCTGTTTTGGAAAGGTGGAGTTGGCAGGGCAGACTGGAGGAAGTGGG |
| | | AGGCTCGGAGGTTAGTAACTACCCCTTACTGAGTGCTTGCTGTAGAGGAAGCATTTTAGTCCTGACGGTG |
| | | ATCCCAGGCCCTGAGTCTTTACTCTGTGCCAGGCACTGTGCTGAGTTCATCTTCAGCACAATCCTATGAG |
| | | ACAGGTATTGTTACCCTCCTCCTCATCACATGGTTGAAGTAGGCAAGGTTCAGAGAGGTCCAATGCCCAA |
| | | GATCACACATGAGGAGGCCAGGACTGGAACCCAAGGCTGACTCTGGACATGAGCACCTGACCTCTCTACC |
| | | TAATGCCTAATGCCTCTCCTGCTGGGAGCCCTTTTTAGAATTTAAGTCTTAAAGGATGGAAGCCCAGAAG |
| | | GAAGCAGAAGCAAGGAAGTGGAAGAGAGGTCCCATGGAAAGGACAGTGCCAAGGACACTGTACAGCCAGC |
| | | CCAATCCTGACCCCTTTTCTTCATCTAGGATTGCCGAGCCCACTAAGGAGCCCATTGTTGATGAAGATGA |
| | | TGATGTGGCTGAAGAAAGACAAAGAATTATTACTGGTGGAAATAAAACTGACATCTTAAGGCTACATGAA |
| | | CTAACCAAGGTAAGGGAATGGGTATGAGTTTGGAGGTGCTGGTTAGATCCACAGTTGGCATGATGTTGCC |
| | | ATTTTCCTTCTATAGAACAATTGATATGCTTATGCAAGCAATTTGGTTCCCAGTTTTATGTAGGGTCATC |
| | | ATCCCTGTGTTATAACTCGTCTTCCAAGAGCATCTAATTCCAATGTGTGTTCCCTGCTATTCATCTCGGG |
| | | CACTGACACAGGGCCTCAGTGAGAATCACTCCAGCTGAGCATCATTCCCTTTTCTGTGTTCTGTTTCTGC |
| | | AGAGCATGGGTCAGCCTCGAGATGTCTCAGTACTCACCACACCTCTGTGCCTGCCCATGTCAATATGTAA |
| | | CCTCCTAGTGCTGGTAGTTTTCTCCTAAACCATCCTTTGCTCTTTGTTCCCTCTTCCCCTCCTTGCTCTC |
| | | ACCCTGTCTCAGTTCTCAGTCCGGTTTCTTCGTATCTTGCAGATTTATCCAGGCACCTCCAGCCCAGCAG |
| | | TGGACAGGCTGTGTGTCGGAGTTCGCCCTGGAGAGGTGGGTACTCTGCAGACCACGTGTGAAAGGCTTCC |
| | | GAACATCAGCTCTTGTGCCTGCCTCTCCTCCCCATAAGGCAGAGCTATTCAATAGGAACATAATGCCATA |
| | | ATGCAAGTCACATATGTAATTTTAAATCTTCCACTAGCCACATGAGAAAAGTAAAAAGAAAATAGGTAAA |
| | | ATTAATTTCATTAGTATTTTTTATTTTACTCAATATAACCAAAATATTATTTCAAAATGTAATTAATAGA |
| | | AAACCTTATTAATGAAATATTTGACAATTTCTCGTTGTTTTTAAGTCTTTGAATCTTTACACTCAGGGCC |
| | | CGTGTCAACTGGGACTTAGATGTGTTTCAAGTGCTTAGTAGCCACATATGGCTCGTGGCCTCTGATGGCA |
| | | GCCCAGGTCTAAAATTCCTCCCCCAGCTCACACACACACTTACCCTGGGGCCTGACATTTTAGACCTTCT |
| | | TGATCTCTAGGGCCAGGCTAGCTCTGTGTTTTCTCCTAGTGCTTTGGCCTCCTGGGAGTGAATGGTGCCG |
| | | GCAAAACAACCACATTCAAGATGCTCACTGGGGACACCACAGTGACCTCAGGGGATGCCACCGTAGCAGG |
| | | CAAGAGGTGAGTATCCTGCTCCTCCTGTCTCAGGGAGTCTCTCACAGGTCCTGTGAGAAGAATAGGAAGG |
| | | GTGATCATCAGACCCTATAGTAGGGTGGCTCTGAGGCCCTGAAAGATCTGTACAGAGAAGGAGGCCTCCC |
| | | AGAGAGCATGGCCCAAAAAGCCCAACACATAGACCCAATGGAAAAGTGAACTGAATTGTGATAGTTAAGA |
| | | GATTCCTCTGTTGGGATGGATTCTTGGAAAGACCTGGGAAGCACTAAGTGTGTGGTTCTTAATCTCTTAG |
| | | AGGTCACGGAACCTTTTAAGCATCTGATGAATATTTGTAGCCTATTCCTATAAAAATGCACCATTGCTTC |
| | | CCATTACCTCCCTCCACACATTTTTACAAAACGTTTCAGGGAGTTTACTGAGCCCCAGGTCACATTTATG |
| | | ATCCTGCAGGAGCTCTTGAATCCCAGGTTAAGAACCCCTGTGATGAATGAAGAATCCTTCCTCTGGGTTG |
| | | AGTTTCTAGATAGGGGCTCATGCATGGGCCTTTGGGGTAGCCTAACCTGCATTGGCTATTTGTAGGCTGA |
| | | TATTTGGCTTTGCCAGACCAAGGAGCATAGAGGGAAAACTGGCGTGTGCCCTTGGATTCTGGAGGGTGAC |
| | | TGCTGCTCTCTGTAATAAAATGTGTTTAAACAGACTGGTCCCCTATGGGCAGGACAGAGAGGATGAGCTC |
| | | TCACTCATCTGCCTCTTTCCTGGCTGCAGGAAAAGCTTGAACAGTAAAACTTCAGCACACACAATAGAGG |
| | | TGCCCAGAGGAAGCCTCTGCCCTGGTTTATAAGTGGAGTTAGGTGCTGCTGACATCTGTCCAGCATCTGC |
| | | TTGACTGGGGCCTCTTCCTCTCTCCTGAAAGCCATCCTCAGCATGGCCCAATGCCCAGTGGGCAGGACGA |
| | | GTCCTGAGCACGCTTCACTGGCTCAGACAGGATGAATTTGATTCTTTGGCCTCCATAGCCAGCCCTACTG |
| | | GGTTTACAGAAAAGGGACAGGCAGGGGTGAAGCCAGGTCATGGCTGAGTCCATCTCAACAGATCCAGCTT |
| | | CACCTGCAAGTGACCACGCAGGTGACTTCCTCATGGTGACAAAAGGAGTCATGGCAGGGTAGAGATATCA |
| | | TACCATGGCAGGGGAAAGATATCATAGAATTTTCCATGAGCACATTTATGAGACATCAAGTTACAACTGT |
| | | GTCCAAGTGAGGCACAGTCTGACATCCAGAAGGTAAAACTGAGCTGGACGCTAGAAAGAAACTATAGGCT |
| | | TAAGACACAGAATTGGGATTATATGGTAGGGTAGCTCCCACTAATTTGGAAACGTACCCTACTTGCTTCC |
| | | CTGAGTAGTTTTAATTGGCCCAGCCATGCCTTTGGTGGCTTTTGTCATTGTGGGGAACTGTAATGGTCTC |
| | | TCTGTACCATCCTATATCATCCATCCTTTATTCATAGACCCTAAGCTATAAGAAGAAAAGGATGAGATTA |
| | | GACTAAATGTCTATGTATAGTTTATTTTCCATCTTGGCAATATATTTTTTAGTGGGGGTGAATATATTAG |
| | | CCAAAGGGAGTTGGTGGAACCCAACTCACTCTACCCCTGCTCCCTGCAGGCCTCTCGCTGTGGGTAGTTA |
| | | TCTGACTGGCTCCTCTTTCATTGCTATCTTTGCCAATAAATACAGATAGAGAAGTTTACTTCCATCGGGA |
| | | CACATGCATCTTTTCTAGTTACTTCCCAAATGTCTGAAAATTATTGATAAATCATGAATCATTTTCTTAA |
| | | ACCTGATCTTCCCTCTGTTTTTAAACTCACATGTGAGGTGATCTGATCCAAAATGAAAGCTGACTTTTGG |
| | | CGTAACAGGGATTCAATTAATCCTAGACATGGAAACATGGAAGAATCTGACAGGATTCAGTTTCTAACCG |
| | | AAGGGCCCCTGTTTTGATTCCCAAATATCCCATGCATTTCTGAAGCCAAATAGGAGAAGAGAAGAAGCAG |
| | | CTTCCTTTTCCCGTTGGCAGAAGCTTCTCCAGCCCTAGCTCTATGGTCATCCCTCCACTCCTTGAAGGAT |
| | | ACTCAGTAATTGCTTTTTTTCTTGCAGTATTTTAACCAATATTTCTGAAGTCCATCAAAATATGGGCTAC |
| | | TGTCCTCAGTTTGATGCAATTGATGAGCTGCTCACAGGACGAGAACATCTTTACCTTTATGCCCGGCTTC |
| | | GAGGTGTACCAGCAGAAGAAATCGAAAAGGTGAAAAATGTTTTGTTGTGGCCACATAGGAGTCTGGTTAA |
| | | TTACAAGCCTGTTTCATGAGAGTGCATTCTCTTGGAGATGAGAAACTGAAGCGTGCTATTCATTCATTCA |
| | | TTCCAACAAATGTTTACTATGTGTCTACTGTGTGCCAAGTACTGTTCTAGAAACCAGGAGTATAGCAGTG |
| | | AACAAGACAGACAAAAAAAAATCCCCACTCTCATATCTAACAAAATGTTGTATGCATTTATCCTCTGACT |
| | | CAGCAATCACACGTCTAAGAGTTTATCCTGAAGATGCATCTCCCACAGTGCAAAATGAATATGTATAAGG |
| | | TGATCCATTGCATTTGTAATTGCAAAATGCTGGAAGTTACCTAAATGTTTAGTCATTGTAGATTGGCTGA |
| | | ATAATTTATGGTACAGACACACAATAAAGTCTTACGCAACTATAAAAAAGAAGAAGAAAAGTCTCAGTAA |
| | | ACTGATATGGAGATATTTCCAGTAAATACTGTTAAATGATAAAAAGCAAAGTGGAAAACAGAACATAGAG |
| | | AACGCTACTTTGTATGTAAGAAAGAAGGAAAAACAAGAAAGTAAACGTATGTCTGCTTACCTTTGCAAAT |
| | | AGAACGTAGAAAGGATAAACCAGAAAACAATGAATTTGGTGATCAACAAGAAGAAAATGGGAAGAAAGAA |
| | | AAATGGGAGGAAACAGTACTTCTGGGGATATATTTTTGTATAGTTTTAATTTTTGGAAGCATGTTAATGT |
| | | TCCACATATTCAAAAAAAATCAGTAAGAATGGGAAGTAGGCAAAAATGAAAACAAAAAGAAAACCTAACA |
| | | CTGACAGCAAACTAAATAAAGTAACCCAATTTTATTTCAAATAAATATCATAATCTTGCAAAAGGGGGAT |
| | | AGAGCTAACACAAACAACTGCTGAACACAGTGTTTGACTCTATATCCTCATTCTTGGGCAGGGTGGAGCG |
| | | GGGGAGAAGAACTACAAATAATTTCTGAGTTCTTTTTAGTTTGTTTTTTATAGTGGTATAGGCAAAGTGA |
| | | TTCTGAAAATTTTAGATGTGTTACAGGATTAAATAAATTAATAAATGTTTTGATGTTATTGGGACCCAGA |
| | | ATTCTCACCGTGGAAGAAGGGACTTACAAATATGGAAAAGGGAAAAGCAAGAAAGAACTGTGAGGTCATG |
| | | GATAGGAACCGGAGGTAGCACTGGGAATTCAGGAATATTTATATGCTTGTGTTTGTGGGTGCATGCAGAT |
| | | GTGTTCATGTTTCATGCACATAGGCATGTATATATAGACATATATTTGCATGTGTGTATCTGTCTTCCGA |
| | | AAGGCTCAAGAAGCAAAAACACCCCAGTAGCCATGAGCACACTTAGCACTCAGGCTTTTGTCTTAATAAC |
| | | ATTCCCCACTAAAAGTAACCCTGATTCCTCCAATAAATGATAAGTTCCAGGGCTGGAATGGCATAGGTAT |
| | | AAAATGAACCTGGAATATCTTATGCCAGAAAGTAAGGAAGTGCTTTTAAAAAAAAAATAAGGGGCTGGGC |
| | | ATGGTGGCTCACACCTGTAATCGCAGCACTTTGGGAGGCCAAGGTAGGAAGATCGCTTGAGCCCAGGAGT |
| | | TCCAGATTAGCCTGTGCAACATAGGGAGACCCTGTCTCTACAAAAAATTAGCAAACAAATTAGCTGGGCC |
| | | TGGTGGTGCACGCCTATAGTCCCAGCTACTCAGGTGGCTGAGGTGGGAGGAATGCTTGAGCCCAGGAGGT |
| | | TGAGGCTGCAGTGAGCTGTGATCAAGCCACTGCTCTCCAGCCTGGGAAACAGAGCAAGACTCTGTCTCTT |
| | | AAAATAATAATAATATAATTTTAAAGAAATAAAAGTAACTCTGTACAGATTGCTTATTGGTTACATGGGA |
| | | GAAACATAATAATTTTACAATGGAGAAATTAGACAGCACCTTAACTGGGTGATCAAAATTAACCATAAGG |
| | | GGCAGATGGACATCTCATGCCCCGAGATGTGATACCCTGTGAAGGACACAATTTCACTTATGTAGAATCC |
| | | AGATTGGAGATATGTAACCTGAATCTTATCATGAGGAAACATCTGACAAGCTCCAAAGAAGGAATATTCC |
| | | TTAAAAAAAAAAAAAGGAGACTGTATTCTTCAAAAACATAAGAGTCATAAAAGACAAAGAAAGAGCTATG |
| | | GAAATATCTCTGATCGCAGGAGGCTAAACAGGCATAATGACTGAATAGCAGACAATAGACTACATCTTGT |
| | | GCAGAAGAGAAAAAAAATGATAGAAGGATATTATTGGACCAACTGACAAAACTGAACTATGAACAGTAGA |
| | | TTAGGTAAATGTATCATAACATTAAGTTTACTGACATTGATAATGTACTGTGGTTATGTAAGAGAAGATC |
| | | TCTATTCTTAGGAAATATGCCCTGAAGTATTTAGGAGTGAAGGGCTGTGATGAGTAATTTACCCTCAAAT |
| | | GGGTCACAAAAAATTGTGTGTGAGAGAGAGAAGGGTTTTATTAGTTAATAATTCTATGAACTATTTTTAT |
| | | TCCTATATGTTTGTGTGAGTTTGAAACTATTTCCAAATAAAAAGTTAAAAATGGAGATTACATTCTAGTG |
| | | GGAGGGATAGACGATCTGTAGATAAATAGGTAAAATATCCAGTACATTAGAGAGTGAAAAGTCCTCAGGG |
| | | AAAAGTAACGCAGGGAGGAACTGCTGGGGCAGGGTTTGCATTTTGAGGTAGGGTGGCCCAGGGAGAGCCT |
| | | GCAGAGGAGAGAACCTGAATGAAGAACTAGAGGTGAGAGAAGGAGCCACGTGCACACCTAGGGAGGAACA |
| | | TTCCAGGCACGGGGGACTAGTATAGAAGGCAGAAGCATGGTGAGCTTGTCTCCAGTGGCTTCCCTAGATC |
| | | CCCTCCTGCGCATGTGCACACACACCTGGTGTCTCTGTCATCGTTCCCTCACAGCACTGTCACGATCTGC |
| | | CAGTATTCTGTTTATTTTGACTGCCACCTCCCCGCAGTCTGAGGATAGCAGCAATGGCTGTGTTCACATT |
| | | GTTCTCCAGTGCCTGGTTCAGTGCCTGGCGTATGGTCAGTGCTCCATAGGTATGTGTCGGATGCACAAGG |
| | | CTTTGGGTGTAACCCTCTTGACGGGTGGGATCAACAGGTCTGGGACTCACCATCTTCTCAAACAGAGCCT |
| | | TCCTCCTCCACTGCTAGCCATGGTCCAGGACGCTGGGCGAGACCCACTGTCTTGCTCTTTGTAAGGCTGA |
| | | AGTCCATTTCCCAGGCGGCTACACCCAACAGATGCTGAGCAGGCTGGGCCACCCTGGGATCCAAGACACA |
| | | GAGAGAAAGAGCCCCTGTCTGGCGCCTGAAGCACATGCCAGAGGACAGGAGCCAGCAGGAGCCTGTTTCA |
| | | GCCTAGCTGGGGATTTCATTCTGGAGGCGTGAGATCTGGGAGCCCAAGGCTTTGAACTGGGGGAGGTTTG |
| | | GGGTGTTTGCTTGTCTTCTCCAAATGGCATTTCTTTCTCTTCCCTAGGTTGCAAACTGGAGTATTAAGAG |
| | | CCTGGGCCTGACTGTCTACGCCGACTGCCTGGCTGGCACGTACAGTGGGGGCAACAAGCGGAAACTCTCC |
| | | ACAGCCATCGCACTCATTGGCTGCCCACCGCTGGTGCTGCTGGTAACTGCGGGCTTGGGCCGCACCAAGG |
| | | GCTTAAACCAAGTGCTGGGTCTCTTGGGTTGGGGAAATAGGTTCTGGGTCGGCAGATTTAGAAACTGCAG |
| | | CAGTTTGGCTTTAGTCTGGACTGTTTCCTGTGTTGCTCATTTTGAGCGATCAGCCCAGTGTTTGGTTCAC |
| | | ACAGCTCCGGAGAAAAACAAGTCACGGCACAGCCTTGACTTGGGACTGCGCACATCCTGCGTTCCCAGGA |
| | | TGTCTCCTGTGGGGCCATCGGCTCACAGCCGGGAAGTTCAGCCCACTCTGCGGCCTGTCGGTGTCTGGTC |
| | | CCCATACAGGAGCACTGAGCTGGGTCAAAGGCTCCTGAGCTGAGCCAGGCCAGGCCTGAGGCCATGCCCA |
| | | CGCAGCCCAAGGATCATGAGGGCACAGGACATAGCGGGAACCAAGGAAGTGACCTGAGTGACCTCCCTGC |
| | | CTTCTGACAAATGTATTTGCAGGATTTTCTTTTTTTGAGGAGAATTCTGTCATTGCCTTAATCCACTTTA |
| | | ATCCCCTCGTGGGCTGAAATGGGCCCAGGATGGACGCCACGCTTCTTTACTCTTGGATCCACCTCCTGCC |
| | | TTCCCTACCCTACACCAGGGTACCCCTGTCTTGCTCAAGTGAGGGGAGTGACTGTGTGCGCCTTCTGTCA |
| | | GCTCATCCTCCACAGGGGAGCCAGCCCAGGGGGAAGCAGTAATCAGAAGGGCCAGCTCCCAGCCTGTGCC |
| | | CCCAACCTTCTCTCCACCCCCCAGGATGAGCCCACCACAGGGATGGACCCCCAGGCACGCCGCATGCTGT |
| | | GGAACGTCATCGTGAGCATCATCAGAGAAGGGAGGGCTGTGGTCCTCACATCCCACAGGCAAGAGATTCC |
| | | CAGGGCTGGGGAAGGTGGGTGGGAATCCTCTCCTGCTCACCTCCTCTCTCCTGCCCCACAGCATGGAAGA |
| | | ATGTGAGGCACTGTGTACCCGGCTGGCCATCATGGTAAAGGGCGCCTTTCGATGTATGGGCACCATTCAG |
| | | CATCTCAAGTCCAAGTAAGCAGATGGTGGGGCGTGCCCCTTGTTGCCTTCTGTGGATCCACCTGGATCCT |
| | | GTGTTCTCCATTGACACTTGGAAGAGTCCTGCTGCTCCGTCATCCCCTGGGGCAGAGGCAGGTGGTGGCT |
| | | GGGCCTCATTCTCCAGCAGCAGATGGAGAAGGCCATCATGCTGATAAGAAACTCCTCTATATTGGCCTAA |
| | | TTTCCTGTGGTCGAAGACTCGCCCAAGTCTCTGGATGGGGCATCTGATCAGGATGCATGCAGAGCCTGGC |
| | | TGGGATGAGGGAGGGCTGCTACCACTGCCTCAATATTTCACCACTTATCTCAACAGATCCGGGACCTGTG |
| | | GCCTATTTACTAAGAGTCCACTCCAATGTAGGAATGGTTAGGAGACCAACTGACTTGAGGACCCATCTTT |
| | | GTTTTTAGAATATTGTATGCTTTTGAGTTTGAAAAAAGACCATATGTTATATGACAAACCAACAATGGCA |
| | | GTAATCTTGAATAGGATTATCCTTATCCTGTACCCACACATTGTAAACTATTGTAGATAATTCCTTATTA |
| | | TTAAGAGTTTGCATGCCAAAGCTAACAGTTTAAGATTATCAGCATATTGCCGTGCTCATTCACGTTCTGA |
| | | TATGCTTTATAACCTAGAAAAGAGCAGAGTTACAATTACTCATTTATTTAACAAACACTTATTAAGAGCT |
| | | CAGAATATAAGTCACTAAGCTGGTTGGTGGGAGGAACAGCACATAACCCACCTTATCTATGCTGAGGTGC |
| | | ATAATCCTGATGCACCCACAGGAGGGTGTTACACAGAAGATGTCATCCTTTCATATGTGTCAGAGCAGAT |
| | | AAATAATTGAGAGAAAGGTCTAATAGATTAGCTGCTTGTGGCAAGTGGACGTTTGACCCATGATTTATTG |
| | | AGCAACTACAACTTGGACACTGCATAGATATCTATAGAAATAGCAGCATGTCAGGTCACCAGACCTGTGT |
| | | CAGCAACTTCCTGTGTCCAACTGCTGGAGAAAGGGAAGTCTCCTATTCCTTTCCCTCCAGCTCCTTAATA |
| | | TCTCCATGATAGAGGGGGTGAGAGGGGAGTGTTCCCTGTGTGGAGGGATGGTGAGTTTTCTGGAGCTGAA |
| | | AGGTAAACAGCCTTTCTCCTCTGCATCTTACTGCAGAGGAGAACAGCCCTAGACTGTGGAGGAAGCTTTG |
| | | GAGTCAGTTATGACTGACACAGGATACCAGGGCATAGGGTACTGACACCCGCTAGCCGTGCACACACTCT |
| | | CTGGTGGACCATCACTCATCCAAGAGAGGGTAACCAGCCATCCTGCTGAAGGAGAAAGAAAGCACCAATG |
| | | GCCCAAGCCCTAGCAGCTCCATTGTTTCAGGAAGCTTCCTCAGGGAAGTGCTGCCTTCCCGAGCCTTTGC |
| | | TCCCACCTGGCCCATCAGCCCTTACCACCACTCAGTATGCACTGGTCCACGTGTCTTTATGGGCAGTCTT |
| | | GGGATCCCCACACTGGGCTAAAACTACCTTTGACGGCCAGGTGCAGTGGCTTACACCTGTAATCCTATCA |
| | | CTTTGGGAAGCTGAGGCAGGTGGATCACTTGAGGTCAGGAGTTCGAGACCAGCCTGGCCAACACGGTGAA |
| | | ACCCTGTCTCTACTAAAAATACAAAAATTAGATGGGCATGGTGGTATGCACCTGTAATCCCACCTACTCG |
| | | GGAAACTGAGGCACAAGAATTGCTTGAACTCAGAAGGCAGAGGTTGCAGTGAATCGAGATCACACCACTG |
| | | CACTCCAGCCTGGGTGAAACAGCAAGACTCTGTCTCAAAAAATAAAATAGGCTGGGCGTGGTGGCTCATG |
| | | CCTGTAATCCCAGCACTTTGGGAGGCCAAGGCGGGCGGATCACTTGAGGTCAGGAGTTTAAGACCAGCCT |
| | | GGCCAACATAGTGAAACCCTGTCTCTACTAAAAATACAAAAAAAAAAAAAAAAATTAGCCGAGTGTGGTG |
| | | GCAGGTGCCTGTAGTTCCAGCCTCTCAGGAGACTGAGGCAGGAGAATTGCTTGAACCCAGGAGGCGGAGG |
| | | TTGCAGTGAGCCAAGATCATGCCACTGTACTCCAGCCTGGGCAACGGTGAGACTGTCTCAAATAAAATAA |
| | | AATAAAATAAAATAAAATAAAATAAAATAAAATAAATAAAATAAAATAAATAAAACTACCTTTGACTTCA |
| | | GCAAGTACGATTATCCCACATTACCATGCAGACATTTGATCTCTAAAAACTGGTATCAAATGATTTCTCC |
| | | AGGGACTACCATGGTTTTTCTCTCCTAGTTTTCAGTATGTACACAGGTCTATGGTATGGGCCTTTAATCC |
| | | CCAGTATTTCTTTTTTTGTTGTTCTTGTTTGGGTTTGTTTCTTGTTTTTCGGTTTTTTTGAGACAGGGTC |
| | | TCACTCTGTCACCCAGGCTGGAGTGCAGTGGCATGATCATGGCTCACTGTAGCCTTGACCTCCTATGCTC |
| | | AAGTGATCCTCCCGCCTCAGCCTCCCAAGTAGCTGGGACCACAGGCATGTGCCACCATGCCCTGCTAATT |
| | | TTCGTAGAGACAGGGTCTTTCTTGTTGCCCAGGCTTATCTTACATTCCTGAGCTCAAGTGATCCTCCCAC |
| | | CTCTACCTCCCAAATTGCTGGGATTTCAGGTGTGAGCCACCAAGCTGAGCTTAATCCCCAAAATTTCTGA |
| | | TGAGTCTACTCCTTATTTTGGGATTACCTTAGGCCCAACCACTAACAGAGGCCTGTCCTGCACTGTGTGC |
| | | ATCCCCTAGATTTGGAGATGGCTATATCGTCACAATGAAGATCAAATCCCCGAAGGACGACCTGCTTCCT |
| | | GACCTGAACCCTGTGGAGCAGTTCTTCCAGGGGAACTTCCCAGGCAGTGTGCAGAGGGAGAGGCACTACA |
| | | ACATGCTCCAGTTCCAGGTCTCCTCCTCCTCCCTGGCGAGGATCTTCCAGCTCCTCCTCTCCCACAAGGA |
| | | CAGCCTGCTCATCGAGGAGTACTCAGTCACACAGACCACACTGGACCAGGCAAGTTGGCCCTGGGGCACC |
| | | GAGAGCTGAGCAAAGACTGGTCCAGAACACCCAGTGTGGGTTGGAATTGCCATAAGAGGGAGGCATAACA |
| | | TTCCCGATTTTTAACAAACTCTTGCCCTCTGTTTATTGGGGTAAAAGCTGATATATCAGAAATTGTTTTC |
| | | TAACAATATTTTTTAGTCATCAGGAAACTTCATTGATTCTTTTTTTTACATTTTCCTTCCCTGTGATGCT |
| | | ATGGTGTGTTATTTCATTCTTGCTCGTTTGTGGTGGTGGTTTTTCCTTCAAATCAGCTTTATTGATGTGT |
| | | AATTAACATACGATGAAACACAGGTTCTTTGGGAGGCCAAGGCAGGAGGATCACTTGAGCCCAGGAGTTT |
| | | AAGACAGGCCCATGTAACAAAGTGAGACTTTGTCTCTACAGAAAAAAAAAAAAAAAATCAGAAAATTAGC |
| | | CAGGCGTGGTGGTGCATGCCTGTGGTCCCATCTACATGGGAGGTTGAGGAAGGAAGATTGCTGGAGCCCA |
| | | GGAGGTCAAGGCTGCAATGAGCTGTGTTCATACCACTGCACTCTAGTCTGGGTGACAGAGCAAGCCCCTG |
| | | TCTCAAAAAAGCAAAACAAAACAAAAACACCTATTTTAAATGTACAGTTTAGTGAGTTTTGATAAACGTG |
| | | CATTCCATGTGTGGTTTTTAAAAATGTAATCACATTTTTTATTGCGGTAAAATATAATAACATAAAATTG |
| | | ACCATGCCAACCATGTTTAAGTGCACAGTGCAGTGGCACTAAGTACATTTACATTGTTGTGCAACCGTTA |
| | | CCACCATCCCCGATAGAACTCTTTCATCTTGCTTCAGTGAAAATCTGTGCCCATTAAACACTAACTCACC |
| | | ACTTACTGCCCCCCTCGCCCTTGGCAACTACTGTTCTACTTTCTGTCTCTAAGGCTCTGACTACTATAGA |
| | | TACCTCATATAAGTGGAATCATACAGTGTTTGTCCTTTTGTGTCTGGCTTATTATGCGAGGACTTAGCAT |
| | | AATGTCCTCAAGGTTCATCCGTGTTGTATCATGTGCCAGAATTTCCTTCCTTTTTCAGGCCGAATAATAT |
| | | TCCTTTGTACGTATATGTGCTACATTTTGTTCATCCATCTATTCATTCATTGATAGACATTTGGGTTGTT |
| | | TCTGGGTTTTGTGTTTTTATATATGTTTTTTTAAAAATAAACATCTTTAGAGACAGTTCAGTAAAGCAGT |
| | | GGAAACAGGGAAGTCTCCATTTAACCCCTGAGGATCTGGCTCACCTGCACCTTCTCATCAGCATTAAGCA |
| | | GAGGGAGGCACGAGCAGGAGCCACCTGCACACTCAATGAGGAGCTGAACAGGGATCAATTACCTTTTTTT |
| | | TTAGTTATTAGGATGCTGCTAGCTGAGAATCTGCCTTGCCTTGATTACCCCAATGTCTGGTGCCCAAGTC |
| | | CCTTGAGTCCTCCAGCAGGAACTCCTGTGGCATCACTCAGGAGTCTAGTCTAAGAAGCTAGCTCTGACCA |
| | | GGGCAGTGGTGGCCAGGCTTCTGTGAGTGGGCCAGCCTCCCCCGGGTAGGACACAAGCCATACCAGCAGG |
| | | GCTGTATGTGAACTGTGGAAAATAGAGAGCAAAGTGGGTAGGTGGGTGTAGGGTGCTGTTTTCCTGGAAA |
| | | TATCTACCTAATCTCGCTCTTCTCTTACCTCTAGGTGTTTGTAAATTTTGCTAAACAGCAGACTGAAAGT |
| | | CATGACCTCCCTCTGCACCCTCGAGCTGCTGGAGCCAGTCGACAAGCCCAGGTACCCCTGCTGCTTATGC |
| | | AGTCCACAGCTTGAGGCAGTTCCTTGGCTCAGAGCCCAGCTGGTTCACTGGGCTTGAGTTGCTCCAAGGC |
| | | TCAGATATGCCTCCTACAGAGAGCCCCACCCACACCACGGTCCCTACCAAGTCCCCACCACATCCTCATC |
| | | ACATCCTTGCTAAGTCCCTGCCACTGTGTGTTCTGTGCTGAAGAACTTTTCATTCAGTAGTTGTAGGGGT |
| | | TCCTATTGTAATCAGGAAACCATCTGGATAGCATGGGAGAGCATTTTTGAAAAGAACTTTCCCATGTTTT |
| | | TGCTTACAGCAAAAAAGCTTGGATTTGGGGAATAAGGAGCAGAGAAGGTAATAGAGAATATTAGAATGTT |
| | | TTGGGTGCTTGACATCTATGTCTGGACATGTGTTTGAGTTTCAAGGGAAGGGACTTAACTGGCACATCAT |
| | | TTCAGTGTCAGACACATTTGGTTAGATCAAGGAATAGCATCTGTTGTAGGAAGAGGGCTCTTTGTTCTTT |
| | | ATAAAAATTACAAGAAGATGGAGAAAGAAGCAATAGGAGGTATGTCTCCTGGCTTGTGATAACTCTTGGA |
| | | ATAGGTGCTTGTAGGTTCCTGCCCTGGCACAGTGCCCCATGTAAGGAGCACACCACCCAAGAAGGAGAGA |
| | | GCTAGAGCAAGTACTGGAGGAGGCACCAGCATCCCAATGCCTTGGCTTAAGCCTGGGATTGTAGAGGGAT |
| | | GAATTAGCCACTCTCTTCTGACTTACCTGGAGAGTAAATCAAATCAAATCAAGAAGCAAGGATATGCAAA |
| | | AACCTTATTTCCCCATAAAGTTTTTATTCTGCCCAGTTTCTGGATTGCAAGAAAAACCAAATACAGCTAA |
| | | TGATTGAAACACTGCTGTCTAAAGCAGTGCTTGTGATGAATTTTTTCCCTTCCTCTTGACCAGCAGAGAC |
| | | CTAATGGCTACTTGGCAAAACTGACTTTGTCTTCCCACCCCTTACCTGCCAGAGGGCCCAGAAATGCCTA |
| | | AGGCTCCTTTAGTTACAGAAAGTTTGCTTTTACTGAGATCTTCCAGCCACTGATTCCCATTTATAGATCT |
| | | GGTGATTGCTGTTGACATCAGTTGAAAATTATTTTTAAAAACCACTTGCAGTTGCAAATCCTTTTTATAA |
| | | CTCTGTAACTCAGAATATAGAATTGGGTAGCAAAATTGTTTCCCAGAATTACCAATGGTCTCCCCACCCC |
| | | TGCCTGGCATGTTCCCTCTTAAAGGACTAATCCCACCACATCACCTCTGGGCCAGGCAGAACATCAGGGG |
| | | TGCTGATGTTCTGTGATCTACAGCAGTTAATTCCAAACTTTTCTCCCTTATTGGATGAGATCATTTTTCT |
| | | ATTGTGTTTTTTACATTTTTGTTCACAAAGATTAGAAAACCTGCAACACACTTATTGGCATATTTTTCTG |
| | | ATAATTTTCATCCAAAACCTAATTCTGACTTTACAACATACTATCTTTACAAAGGTTTGCAAAAATTCTT |
| | | TCATATAGCATTGTATATGTCTGTCATGAAATAATAGTAAGTATATTATTGTTTACATTATACCACTTCA |
| | | AAATAATTTCCTTTAAAGTATTCTTCAAACAAGAAAAAGGCAATTTCTCTCAAGAAGTTTTAGAGAGAAT |
| | | TTACAACTTGCTCCTAAGCAAATGTGAGAACTTCAGGAGGTTCATCTGGCCATTGGCTTTACAACTCCAA |
| | | ATTGTGAGCCAGGACCACACAGATATTTCTCTAGAAATCAGCGTTTGCTTACCAAGAACATTTTTACTCT |
| | | CCAAAGGACTCCATCCTGGAAAACATGTTTTGGGATAAGGTCTTATGCAATCTTATACTCTGTTATTAAA |
| | | ACCAGTGAGGGTCAAGGTGTTAATAGATTAAGTAGTGACAGATGATCAGACAACTTAGAAACATCCTAAA |
| | | TAGGTTAATAATTATGTGACCATCGCATGTGCATTCCCAAATTAGGAACAACTCAGATCAATTTCTAATC |
| | | CTTATTCTTACACTGTTCCAGTTCCCCCATATAACTCGTATCTTTGTGTTAGTTTCAGAAGTTTCTGAAG |
| | | TACCCTCAGCCTTGATGGGGATCCTCGCACCACCTCAAATCCTGTTCTCAGCCCTAAGAACTGTGTTAGT |
| | | CATCCTCTTAAGAGGATGTGTGATTTTAAATCAGATAATGGGATAAACCACATTTCGTCTAGACTGGTCA |
| | | GGCCTTTGTCCAGTCCCCTCCTCGCCCACACTACCCCAGCTCCACAGCGGGCATTGGTTCAGGAATTCAA |
| | | CCCACACTTTATAACTGGAGACAGTATCTCTCCAGTTAAAAAGGTCACCTTGGTGTCCGCTTCTCAAGGA |
| | | ACATGGACATCTTTATTAATCAAAGCCCAAGCTTTGATCTGGAGCCTAATATCCTGCACTCCAGCTCTCA |
| | | TCTCTCCCCTCCCCCAGTCACACTTTCATGCTTCCCAGAGCCACCCCTACAGGAAGTGGTCAAGGGAATT |
| | | CTATACCTCAGGGCTGACCTAAATTAGGATTTCTTGGCTTTTAAGATAATGGTAACTTTCTTAAGCTAAA |
| | | AAAGCCCCAAAAGACCCTGTAAGAGCCCTTGGAAACAGCACCATGGGTGTAGCTTCCCCCCAGGATGTAA |
| | | GCATGTATGCACACATCTCGTATGTGTGTCTTTGTAACAAATGCCTGGATCTTAGTACCAGGGAGACCTG |
| | | ATTCATAGATTTCATAGAGAAGGAGAGAAAGATGGCCCATAACCTGGGTGATCTGACAGAATCACAGTGC |
| | | CCTCAGCTGAGTGCCCTTCAGAAATTGATTGACAACTGTTTAGCTTTTGAAATCTAAAAGTAGTACAGCA |
| | | TCTCAGAAAACCAAGATGACGCGAGTCCATGTGATCTCCTTCCACAGGACTGATCTTTCACACCGCTCGT |
| | | TCCTGCAGCCAGAAAGGAACTCTGGGCAGCTGGAGGCGCAGGAGCCTGTGCCCATATGGTCATCCAAATG |
| | | GACTGGCCAGCGTAAATGACCCCACTGCAGCAGAAAACAAACACACGAGGAGCATGCAGCGAATTCAGAA |
| | | AGAGGTCTTTCAGAAGGAAACCGAAACTGACTTGCTCACCTGGAACACCTGATGGTGAAACCAAACAAAT |
| | | ACAAAATCCTTCTCCAGACCCCAGAACTAGAAACCCCGGGCCATCCCACTAGCAGCTTTGGCCTCCATAT |
| | | TGCTCTCATTTCAAGCAGATCTGCTTTTCTGCATGTTTGTCTGTGTGTCTGCGTTGTGTGTGATTTTCAT |
| | | GGAAAAATAAAATGCAAATGCACTCATCACAAACTATCCTAATTCACAGTCTCCCTGGTGTGCACCACCT |
| | | AGTATAGTTTTAGACATTCTTTAGATGGGTGCATAGCTCCTTGTCAGTCCCATGCACTTCTGTGAGTGTT |
| | | ACTGCCTCAGGACTGCTCGTTCTGGCAAGATTCTGCAACACTAGGTTGGAAGTGAATGGACTAGTCTTAA |
| | | TGTTCCATGTCAAGTCTTTGTAGAGTTTGAAGAAAACACCCAACTAGTAATGCCTGTAAACATTATCCAT |
| | | TGTCAGCTGGGTATTACTGACTTTAAGTTTCGTGTCTGTTTGCCCAGCTTATTTGAGTGTTTACCTCACA |
| | | AGTGTAATTAGGACAGGAGACAAAGAGGCATGCACAGGCGAGAGTTGGTCCTTGGTTGGACGTGAGACCC |
| | | GACAGGACTTTGTAACCATTTGAAGAGGTCAGGACCTCATTCTCATGCTGCTGTGCCTTTTCTGCAGTGC |
| | | TACCATGTGCATCTTCTGCAGTGGTGTTAGAAGGGAATGAAGGCCGGGCGCAGTAGCTCACGCCTGTAAT |
| | | CCCAGCACTTTGGGAGCCTGAGGTGGGCAGATCACAAGATCAGAAGATCAAGACCATCCTGACTAACACA |
| | | GTGAGACCCCGTGTCTACTAAAACTATGAAAAATTAGCTGAGCATGGTGGCACATGCCTGTAGTCCCAGC |
| | | TACTCAGGAGGCTGAGGCAGGAGAATCGCTTGAACCCAGGAGGCAGAGGTTGCAGTGAGCTGAGATTGTG |
| | | CCACTGCACACCAGCCTGGCGACAGAGCAAGACTCCATCTAAAAATAAATAAATAAATAAAAAGGAATGA |
| | | AGATGGACATCTAGTTCACATAAATGCTCATCAAGATTCAGAAAATAATAATTTTAAACCAAACATTTCC |
| | | AGAGACTGTGGATGAGGAGAACAAGTGGGTTTCCTTGTCAGGGCAGTCTCCCCTCCATGACCTAGAGATG |
| | | GGCTTCGTCGTAAACATGCTTTCATTATACTGAGGATAGACTCCAGAGTCGGGGTGAGGCAAGAGGAAAA |
| | | GGGGAGAGATGCTGAGGCCCAGGAACTGTTGCTGAGAAGAGGATGAGAAAGAAAGTGAGCACAAGGTGCT |
| | | AAAATTTTTGTTTCAGCTGTGCTTGTTTAGAGGCAGACAGAGGGCAAGGGCTACACAACTTTAAGTCCTG |
| | | CACACCTCCTGGCTTGCCACTTTGCACCTTCTAGATGCTAGGCAAACAACTGCCCATAGAGACTATAAAA |
| | | CTACTTTGGTAACCCCGCAGCTTCATCTGGTGTTGACTTTTTCTTTTAAGTTACCAAGGACCAAAACTGT |
| | | AAGACCATTTTAGCTAGGGCTAGGATAGAGGTTGGGAAAGCCCAGCACACTGCTTGCATCACACTGCTGC |
| | | ACCCTGGCTGGTTTCATAATTTAAATTAGCAACTGCAATATCACAGGAAAGAAGACTACACTCTTCGGGG |
| | | CTGCTTAGGAAAAGAAAACTAAAAAAAGACTATGTAGGGGAGGTGGTTTAGCAGCCATTCTGTTTGGCTG |
| | | TGAGGGTTTCGGAAAGGCATCATGAACTGGGAAGAGTCGATGCAGGTAAAATCTGCACACCCCCTTAAGG |
| | | AAAAATCTCAGTTTACCTTTTGTCTCCACTACACCTGAGGTCTGTGTCTTTTCATCCTGTTTTTTCCAGC |
| | | TCCTGGCACACAGAAAATGTTCAACTAATACCCACCAAACTGAAAACCCAGCAAACTATGAAAACTCAGC |
| | | AAGAAAAATAGACAGAAAAGAAGTGGGTCCAAGAAAATGATGCCTCCAAAAAGCAGCAAAGGGCAAGTGG |
| | | AGCAGGAGGATGCCGTGCTTTAAAAACAGCCACAGGCCGGGTGTGGTGGCTCACGTCTAATCCTAGCACT |
| | | TTGGGAGGCCGAGGCGGGCGGATTGCCTGAGCTCAGGAGTTCGAGACCAGCTGGCCAATGTGGTGAAAGC |
| | | CCGTCTCTATTAAAATACAAAAAAAAAAAAAAGAAAGAAAGAAAATTAGCCAGGCGTGGTGGTGGGTGCC |
| | | TGT |
| 17 | ABCA4 | GgacacAgcGtccggAgccagAggcgcTctTaacggcgtTtaTgtcctTtgctgTctGag |
| | DNA | GggCctCagCtcTgaCcaAtcTggTctTcgTgtGgtCatTagCatGggCttCgtGagAca |
| | | GatAcaGctTttGctCtgGaaGaaCtgGacCctGcgGaaAagGcaAaaGatTcgCttTgt |
| | | GgtGgaActCgtGtgGccTttAtcTttAttTctGgtCttGatCtgGttAagGaaTgcCaa |
| | | CccActCtaCagCcaTcaTgaAtgCcaTttCccCaaCaaGgcGatGccCtcAgcAggAat |
| | | GctGccGtgGctCcaGggGatCttCtgCaaTgtGaaCaaTccCtgTttTcaAagCccCac |
| | | CccAggAgaAtcTccTggAatTgtGtcAaaCtaTaaCaaCtcCatCttGgcAagGgtAta |
| | | TcgAgaTttTcaAgaActCctCatGaaTgcAccAgaGagCcaGcaCctTggCcgTatTtg |
| | | GacAgaGctAcaCatCttGtcCcaAttCatGgaCacCctCcgGacTcaCccGgaGagAat |
| | | TgcAggAagAggAatAcgAatAagGgaTatCttGaaAgaTgaAgaAacActGacActAtt |
| | | TctCatTaaAaaCatCggCctGtcTgaCtcAgtGgtCtaCctTctGatCaaCtcTcaAgt |
| | | CcgTccAgaGcaGttCgcTcaTggAgtCccGgaCctGgcGctGaaGgaCatCgcCtgCag |
| | | CgaGgcCctCctGgaGcgCttCatCatCttCagCcaGagAcgCggGgcAaaGacGgtGcg |
| | | CtaTgcCctGtgCtcCctCtcCcaGggCacCctAcaGtgGatAgaAgaCacTctGtaTgc |
| | | CaaCgtGgaCttCttCaaGctCttCcgTgtGctTccCacActCctAgaCagCcgTtcTca |
| | | AggTatCaaTctGagAtcTtgGggAggAatAttAtcTgaTatGtcAccAagAatTcaAga |
| | | GttTatCcaTcgGccGagTatGcaGgaCttGctGtgGgtGacCagGccCctCatGcaGaa |
| | | TggTggTccAgaGacCttTacAaaGctGatGggCatCctGtcTgaCctCctGtgTggCta |
| | | CccCgaGggAggTggCtcTcgGgtGctCtcCttCaaCtgGtaTgaAgaCaaTaaCtaTaa |
| | | GgcCttTctGggGatTgaCtcCacAagGaaGgaTccTatCtaTtcTtaTgaCagAagAac |
| | | AacAtcCttTtgTaaTgcAttGatCcaGagCctGgaGtcAaaTccTttAacCaaAatCgc |
| | | TtgGagGgcGgcAaaGccTttGctGatGggAaaAatCctGtaCacTccTgaTtcAccTgc |
| | | AgcAcgAagGatActGaaGaaTgcCaaCtcAacTttTgaAgaActGgaAcaCgtTagGaa |
| | | GttGgtCaaAgcCtgGgaAgaAgtAggGccCcaGatCtgGtaCttCttTgaCaaCagCac |
| | | AcaGatGaaCatGatCagAgaTacCctGggGaaCccAacAgtAaaAgaCttTttGaaTag |
| | | GcaGctTggTgaAgaAggTatTacTgcTgaAgcCatCctAaaCttCctCtaCaaGggCcc |
| | | TcgGgaAagCcaGgcTgaCgaCatGgcCaaCttCgaCtgGagGgaCatAttTaaCatCac |
| | | TgaTcgCacCctCcgCctGgtCaaTcaAtaCctGgaGtgCttGgtCctGgaTaaGttTga |
| | | AagCtaCaaTgaTgaAacTcaGctCacCcaAcgTgcCctCtcTctActGgaGgaAaaCat |
| | | GttCtgGgcCggAgtGgtAttCccTgaCatGtaTccCtgGacCagCtcTctAccAccCca |
| | | CgtGaaGtaTaaGatCcgAatGgaCatAgaCgtGgtGgaGaaAacCaaTaaGatTaaAga |
| | | CagGtaTtgGgaTtcTggTccCagAgcTgaTccCgtGgaAgaTttCcgGtaCatCtgGgg |
| | | CggGttTgcCtaTctGcaGgaCatGgtTgaAcaGggGatCacAagGagCcaGgtGcaGgc |
| | | GgaGgcTccAgtTggAatCtaCctCcaGcaGatGccCtaCccCtgCttCgtGgaCgaTtc |
| | | TttCatGatCatCctGaaCcgCtgTttCccTatCttCatGgtGctGgcAtgGatCtaCtc |
| | | TgtCtcCatGacTgtGaaGagCatCgtCttGgaGaaGgaGttGcgActGaaGgaGacCtt |
| | | GaaAaaTcaGggTgtCtcCaaTgcAgtGatTtgGtgTacCtgGttCctGgaCagCttCtc |
| | | CatCatGtcGatGagCatCttCctCctGacGatAttCatCatGcaTggAagAatCctAca |
| | | TtaCagCgaCccAttCatCctCttCctGttCttGttGgcTttCtcCacTgcCacCatCat |
| | | GctGtgCttTctGctCagCacCttCttCtcCaaGgcCagTctGgcAgcAgcCtgTagTgg |
| | | TgtCatCtaTttCacCctCtaCctGccAcaCatCctGtgCttCgcCtgGcaGgaCcgCat |
| | | GacCgcTgaGctGaaGaaGgcTgtGagCttActGtcTccGgtGgcAttTggAttTggCac |
| | | TgaGtaCctGgtTcgCttTgaAgaGcaAggCctGggGctGcaGtgGagCaaCatCggGaa |
| | | CagTccCacGgaAggGgaCgaAttCagCttCctGctGtcCatGcaGatGatGctCctTga |
| | | TgcTgcTgtCtaTggCttActCgcTtgGtaCctTgaTcaGgtGttTccAggAgaCtaTgg |
| | | AacCccActTccTtgGtaCttTctTctAcaAgaGtcGtaTtgGctTggCggTgaAggGtg |
| | | TtcAacCagAgaAgaAagAgcCctGgaAaaGacCgaGccCctAacAgaGgaAacGgaGga |
| | | TccAgaGcaCccAgaAggAatAcaCgaCtcCttCttTgaAcgTgaGcaTccAggGtgGgt |
| | | TccTggGgtAtgCgtGaaGaaTctGgtAaaGatTttTgaGccCtgTggCcgGccAgcTgt |
| | | GgaCcgTctGaaCatCacCttCtaCgaGaaCcaGatCacCgcAttCctGggCcaCaaTgg |
| | | AgcTggGaaAacCacCacCttGtcCatCctGacGggTctGttGccAccAacCtcTggGac |
| | | TgtGctCgtTggGggAagGgaCatTgaAacCagCctGgaTgcAgtCcgGcaGagCctTgg |
| | | CatGtgTccAcaGcaCaaCatCctGttCcaCcaCctCacGgtGgcTgaGcaCatGctGtt |
| | | CtaTgcCcaGctGaaAggAaaGtcCcaGgaGgaGgcCcaGctGgaGatGgaAgcCatGtt |
| | | GgaGgaCacAggCctCcaCcaCaaGcgGaaTgaAgaGgcTcaGgaCctAtcAggTggCat |
| | | GcaGagAaaGctGtcGgtTgcCatTgcCttTgtGggAgaTgcCaaGgtGgtGatTctGga |
| | | CgaAccCacCtcTggGgtGgaCccTtaCtcGagAcgCtcAatCtgGgaTctGctCctGaa |
| | | GtaTcgCtcAggCagAacCatCatCatGtcCacTcaCcaCatGgaCgaGgcCgaCctCct |
| | | TggGgaCcgCatTgcCatCatTgcCcaGggAagGctCtaCtgCtcAggCacCccActCtt |
| | | CctGaaGaaCtgCttTggCacAggCttGtaCttAacCttGgtGcgCaaGatGaaAaaCat |
| | | CcaGagCcaAagGaaAggCagTgaGggGacCtgCagCtgCtcGtcTaaGggTttCtcCac |
| | | CacGtgTccAgcCcaCgtCgaTgaCctAacTccAgaAcaAgtCctGgaTggGgaTgtAaa |
| | | TgaGctGatGgaTgtAgtTctCcaCcaTgtTccAgaGgcAaaGctGgtGgaGtgCatTgg |
| | | TcaAgaActTatCttCctTctTccAaaTaaGaaCttCaaGcaCagAgcAtaTgcCagCct |
| | | TttCagAgaGctGgaGgaGacGctGgcTgaCctTggTctCagCagTttTggAatTtcTga |
| | | CacTccCctGgaAgaGatTttTctGaaGgtCacGgaGgaTtcTgaTtcAggAccTctGtt |
| | | TgcGggTggCgcTcaGcaGaaAagAgaAaaCgtCaaCccCcgAcaCccCtgCttGggTcc |
| | | CagAgaGaaGgcTggAcaGacAccCcaGgaCtcCaaTgtCtgCtcCccAggGgcGccGgc |
| | | TgcTcaCccAgaGggCcaGccTccCccAgaGccAgaGtgCccAggCccGcaGctCaaCac |
| | | GggGacAcaGctGgtCctCcaGcaTgtGcaGgcGctGctGgtCaaGagAttCcaAcaCac |
| | | CatCcgCagCcaCaaGgaCttCctGgcGcaGatCgtGctCccGgcTacCttTgtGttTtt |
| | | GgcTctGatGctTtcTatTgtTatCccTccTttTggCgaAtaCccCgcTttGacCctTca |
| | | CccCtgGatAtaTggGcaGcaGtaCacCttCttCagCatGgaTgaAccAggCagTgaGca |
| | | GttCacGgtActTgcAgaCgtCctCctGaaTaaGccAggCttTggCaaCcgCtgCctGaa |
| | | GgaAggGtgGctTccGgaGtaCccCtgTggCaaCtcAacAccCtgGaaGacTccTtcTgt |
| | | GtcCccAaaCatCacCcaGctGttCcaGaaGcaGaaAtgGacAcaGgtCaaCccTtcAcc |
| | | AtcCtgCagGtgCagCacCagGgaGaaGctCacCatGctGccAgaGtgCccCgaGggTgc |
| | | CggGggCctCccGccCccCcaGagAacAcaGcgCagCacGgaAatTctAcaAgaCctGac |
| | | GgaCagGaaCatCtcCgaCttCttGgtAaaAacGtaTccTgcTctTatAagAagCagCtt |
| | | AaaGagCaaAttCtgGgtCaaTgaAcaGagGtaTggAggAatTtcCatTggAggAaaGct |
| | | CccAgtCgtCccCatCacGggGgaAgcActTgtTggGttTttAagCgaCctTggCcgGat |
| | | CatGaaTgtGagCggGggCccTatCacTagAgaGgcCtcTaaAgaAatAccTgaTttCct |
| | | TaaAcaTctAgaAacTgaAgaCaaCatTaaGgtGtgGttTaaTaaCaaAggCtgGcaTgc |
| | | CctGgtCagCttTctCaaTgtGgcCcaCaaCgcCatCttAcgGgcCagCctGccTaaGga |
| | | CagGagCccCgaGgaGtaTggAatCacCgtCatTagCcaAccCctGaaCctGacCaaGga |
| | | GcaGctCtcAgaGatTacAgtGctGacCacTtcAgtGgaTgcTgtGgtTgcCatCtgCgt |
| | | GatTttCtcCatGtcCttCgtCccAgcCagCttTgtCctTtaTttGatCcaGgaGcgGgt |
| | | GaaCaaAtcCaaGcaCctCcaGttTatCagTggAgtGagCccCacCacCtaCtgGgtGac |
| | | CaaCttCctCtgGgaCatCatGaaTtaTtcCgtGagTgcTggGctGgtGgtGggCatCtt |
| | | CatCggGttTcaGaaGaaAgcCtaCacTtcTccAgaAaaCctTccTgcCctTgtGgcAct |
| | | GctCctGctGtaTggAtgGgcGgtCatTccCatGatGtaCccAgcAtcCttCctGttTga |
| | | TgtCccCagCacAgcCtaTgtGgcTttAtcTtgTgcTaaTctGttCatCggCatCaaCag |
| | | CagTgcTatTacCttCatCttGgaAttAttTgaGaaTaaCcgGacGctGctCagGttCaa |
| | | CgcCgtGctGagGaaGctGctCatTgtCttCccCcaCttCtgCctGggCcgGggCctCat |
| | | TgaCctTgcActGagCcaGgcTgtGacAgaTgtCtaTgcCcgGttTggTgaGgaGcaCtc |
| | | TgcAaaTccGttCcaCtgGgaCctGatTggGaaGaaCctGttTgcCatGgtGgtGgaAgg |
| | | GgtGgtGtaCttCctCctGacCctGctGgtCcaGcgCcaCttCttCctCtcCcaAtgGat |
| | | TgcCgaGccCacTaaGgaGccCatTgtTgaTgaAgaTgaTgaTgtGgcTgaAgaAagAca |
| | | AagAatTatTacTggTggAaaTaaAacTgaCatCttAagGctAcaTgaActAacCaaGat |
| | | TtaTccAggCacCtcCagCccAgcAgtGgaCagGctGtgTgtCggAgtTcgCccTggAga |
| | | GtgCttTggCctCctGggAgtGaaTggTgcCggCaaAacAacCacAttCaaGatGctCac |
| | | TggGgaCacCacAgtGacCtcAggGgaTgcCacCgtAgcAggCaaGagTatTttAacCaa |
| | | TatTtcTgaAgtCcaTcaAaaTatGggCtaCtgTccTcaGttTgaTgcAatTgaTgaGct |
| | | GctCacAggAcgAgaAcaTctTtaCctTtaTgcCcgGctTcgAggTgtAccAgcAgaAga |
| | | AatCgaAaaGgtTgcAaaCtgGagTatTaaGagCctGggCctGacTgtCtaCgcCgaCtg |
| | | CctGgcTggCacGtaCagTggGggCaaCaaGcgGaaActCtcCacAgcCatCgcActCat |
| | | TggCtgCccAccGctGgtGctGctGgaTgaGccCacCacAggGatGgaCccCcaGgcAcg |
| | | CcgCatGctGtgGaaCgtCatCgtGagCatCatCagAgaAggGagGgcTgtGgtCctCac |
| | | AtcCcaCagCatGgaAgaAtgTgaGgcActGtgTacCcgGctGgcCatCatGgtAaaGgg |
| | | CgcCttTcgAtgTatGggCacCatTcaGcaTctCaaGtcCaaAttTggAgaTggCtaTat |
| | | CgtCacAatGaaGatCaaAtcCccGaaGgaCgaCctGctTccTgaCctGaaCccTgtGga |
| | | GcaGttCttCcaGggGaaCttCccAggCagTgtGcaGagGgaGagGcaCtaCaaCatGct |
| | | CcaGttCcaGgtCtcCtcCtcCtcCctGgcGagGatCttCcaGctCctCctCtcCcaCaa |
| | | GgaCagCctGctCatCgaGgaGtaCtcAgtCacAcaGacCacActGgaCcaGgtGttTgt |
| | | AaaTttTgcTaaAcaGcaGacTgaAagTcaTgaCctCccTctGcaCccTcgAgcTgcTgg |
| | | AgcCagTcgAcaAgcCcaGgaCtgAtcTttCacAccGctCgtTccTgcAgcCagAaaGga |
| | | ActCtgGgcAgcTggAggCgcAggAgcCtgTgcCcaTatGgtCatCcaAatGgaCtgGcc |
| | | AgcGtaAatGacCccActGcaGcaGaaAacAaaCacAcgAggAgcAtgCagCgaAttCag |
| | | AaaGagGtcTttCagAagGaaAccGaaActGacTtgCtcAccTggAacAccTgaTggTga |
| | | AacCaaAcaAatAcaAaaTccTtcTccAgaCccCagAacTagAaaCccCggGccAtcCca |
| | | CtaGcaGctTtgGccTccAtaTtgCtcTcaTttCaaGcaGatCtgCttTtcTgcAtgTtt |
| | | GtcTgtGtgTctGcgTtgTgtGtgAttTtcAtgGaaAaaTaaAatGcaAatGcaCtcAtc |
| | | AcaAacta |
| 18 | ABCA4 | MGFVRQIQLLLWKNWTLRKRQKIRFVVELVWPLSLFLVLIWLRN |
| | protein | ANPLYSHHECHFPNKAMPSAGMLPWLQGIFCNVNNPCFQSPTPGESPGIVSNYNNSIL |
| | | ARVYRDFQELLMNAPESQHLGRINTELHILSQFMDTLRTHPERIAGRGIRIRDILKDE |
| | | ETLTLFLIKNIGLSDSVVYLLINSQVRPEQFAHGVPDLALKDIACSEALLERFIIFSQ |
| | | RRGAKTVRYALCSLSQGTLQWIEDTLYANVDFFKLFRVLPTLLDSRSQGINLRSWGGI |
| | | LSDMSPRIQEFIHRPSMQDLLWVTRPLMQNGGPETFTKLMGILSDLLCGYPEGGGSRV |
| | | LSFNWYEDNNYKAFLGIDSTRKDPIYSYDRRTTSFCNALIQSLESNPLTKIAWRAAKP |
| | | LLMGKILYTPDSPAARRILKNANSTFEELEHVRKLVKAWEEVGPQIWYFFDNSTQMNM |
| | | IRDTLGNPTVKDFLNRQLGEEGITAEAILNFLYKGPRESQADDMANFDWRDIFNITDR |
| | | TLRLVNQYLECLVLDKFESYNDETQLTQRALSLLEENMFWAGVVFPDMYPWTSSLPPH |
| | | VKYKIRMDIDVVEKTNKIKDRYWDSGPRADPVEDFRYINGGFAYLQDMVEQGITRSQV |
| | | QAEAPVGIYLQQMPYPCFVDDSFMIILNRCFPIFMVLAWIYSVSMTVKSIVLEKELRL |
| | | KETLKNQGVSNAVIWCTWFLDSFSIMSMSIFLLTIFIMHGRILHYSDPFILFLFLLAF |
| | | STATIMLCFLLSTFFSKASLAAACSGVIYFTLYLPHILCFAWQDRMTAELKKAVSLLS |
| | | PVAFGFGTEYLVRFEEQGLGLQWSNIGNSPTEGDEFSFLLSMQMMLLDAAVYGLLAWY |
| | | LDQVFPGDYGTPLPWYFLLQESYWLGGEGCSTREERALEKTEPLTEETEDPEHPEGIH |
| | | DSFFEREHPGWVPGVCVKNLVKIFEPCGRPAVDRLNITFYENQITAFLGHNGAGKTTT |
| | | LSILTGLLPPTSGTVLVGGRDIETSLDAVRQSLGMCPQHNILFHHLTVAEHMLFYAQL |
| | | KGKSQEEAQLEMEAMLEDTGLHHKRNEEAQDLSGGMQRKLSVAIAFVGDAKVVILDEP |
| | | TSGVDPYSRRSIWDLLLKYRSGRTIIMSTHHMDEADLLGDRIAIIAQGRLYCSGTPLF |
| | | LKNCFGTGLYLTLVRKMKNIQSQRKGSEGTCSCSSKGFSTTCPAHVDDLTPEQVLDGD |
| | | VNELMDVVLHHVPEAKLVECIGQELIFLLPNKNFKHRAYASLFRELEETLADLGLSSF |
| | | GISDTPLEEIFLKVTEDSDSGPLFAGGAQQKRENVNPRHPCLGPREKAGQTPQDSNVC |
| | | SPGAPAAHPEGQPPPEPECPGPQLNTGTQLVLQHVQALLVKRFQHTIRSHKDFLAQIV |
| | | LPATFVFLALMLSIVIPPFGEYPALTLHPWIYGQQYTFFSMDEPGSEQFTVLADVLLN |
| | | KPGFGNRCLKEGWLPEYPCGNSTPWKTPSVSPNITQLFQKQKWTQVNPSPSCRCSTRE |
| | | KLTMLPECPEGAGGLPPPQRTQRSTEILQDLTDRNISDFLVKTYPALIRSSLKSKFWV |
| | | NEQRYGGISIGGKLPVVPITGEALVGFLSDLGRIMNVSGGPITREASKEIPDFLKHLE |
| | | TEDNIKVWFNNKGWHALVSFLNVAHNAILRASLPKDRSPEEYGITVISQPLNLTKEQL |
| | | SEITVLTTSVDAVVAICVIFSMSFVPASFVLYLIQERVNKSKHLQFISGVSPTTYWVT |
| | | NFLWDIMNYSVSAGLVVGIFIGFQKKAYTSPENLPALVALLLLYGWAVIPMMYPASFL |
| | | FDVPSTAYVALSCANLFIGINSSAITFILELFENNRTLLRFNAVLRKLLIVFPHFCLG |
| | | RGLIDLALSQAVTDVYARFGEEHSANPFHWDLIGKNLFAMVVEGVVYFLLTLLVQRHF |
| | | FLSQWIAEPTKEPIVDEDDDVAEERQRIITGGNKTDILRLHELTKIYPGTSSPAVDRL |
| | | CVGVRPGECFGLLGVNGAGKTTTFKMLTGDTTVTSGDATVAGKSILTNISEVHQNMGY |
| | | CPQFDAIDELLTGREHLYLYARLRGVPAEEIEKVANWSIKSLGLTVYADCLAGTYSGG |
| | | NKRKLSTAIALIGCPPLVLLDEPTTGMDPQARRMLWNVIVSIIREGRAVVLTSHSMEE |
| | | CEALCTRLAIMVKGAFRCMGTIQHLKSKFGDGYIVTMKIKSPKDDLLPDLNPVEQFFQ |
| | | GNFPGSVQRERHYNMLQFQVSSSSLARIFQLLLSHKDSLLIEEYSVTQTTLDQVFVNF |
| | | AKQQTESHDLPLHPRAAGASRQAQD |
| 19 | C³- | CTCTTCGgtatcacaggagaatttcagGGAGACATTGATTATTGACTAGTTATTAATAGTAATCAATTACGGGGT |
| | ABCA4 | CATTAGTTCATAGCCCATATATGGAGTTCCGCGTTACATAACTTACGGTAAATGGCCCGCCTGGCTGACCGCCCA |
| | | ACGACCCCCGCCCATTGACGTCAATAATGACGTATGTTCCCATAGTAACGCCAATAGGGACTTTCCATTGACGTC |
| | | AATGGGTGGAGTATTTACGGTAAACTGCCCACTTGGCAGTACATCAAGTGTATCATATGCCAAGTACGCCCCCTA |
| | | TTGACGTCAATGACGGTAAATGGCCCGCCTGGCATTATGCCCAGTACATGACCTTATGGGACTTTCCTACTTGGC |
| | | AGTACATCTACGTATTAGTCATCGCTATTACCATGTCGAGGTGAGCCCCACGTTCTGCTTCACTCTCCCCATCTC |
| | | CCCCCCCTCCCCACCCCCAATTTTGTATTTATTTATTTTTTAATTATTTTGTGCAGCGATGGGGGCGGGGGGGGG |
| | | GGGGGGGCGCGCGCCAGGCGGGGCGGGGCGGGGCGAGGGGCGGGGCGGGGCGAGGCGGAGAGGTGCGGCGGCAGC |
| | | CAATCAGAGCGGCGCGCTCCGAAAGTTTCCTTTTATGGCGAGGCGGCGGCGGCGGCGGCCCTATAAAAAGCGAAG |
| | | CGCGCGGCGGGCGGGAGTCGCTGCGACGCTGCCTTCGCCCCGTGCCCCGCTCCGCCGCCGCCTCGCGCCGCCCGC |
| | | CCCGGCTCTGACTGACCGCGTTACTCCCACAGGTGAGCGGGCGGGACGGCCCTTCTCCTCCGGGCTGTAATTAGC |
| | | GCTTGGTTTAATGACGGCTTGTTTCTTTTCTGTGGCTGCGTGAAAGCCTTGAGGGGCTCCGGGAGGGCCCTTTGT |
| | | GCGGGGGGAGCGGCTCGGGGGGTGCGTGCGTGTGTGTGTGCGTGGGGAGCGCCGCGTGCGGCTCCGCGCTGCCCG |
| | | GCGGCTGTGAGCGCTGCGGGCGCGGCGCGGGGCTTTGTGCGCTCCGCAGTGTGCGCGAGGGGAGCGCGGCCGGGG |
| | | GCGGTGCCCCGCGGTGCGGGGGGGGCTGCGAGGGGAACAAAGGCTGCGTGCGGGGTGTGTGCGTGGGGGGGTGAG |
| | | CAGGGGGTGTGGGCGCGTCGGTCGGGCTGCAACCCCCCCTGCACCCCCCTCCCCGAGTTGCTGAGCACGGCCCGG |
| | | CTTCGGGTGCGGGGCTCCGTACGGGGCGTGGCGCGGGGCTCGCCGTGCCGGGCGGGGGGTGGCGGCAGGTGGGGG |
| | | TGCCGGGCGGGGCGGGGCCGCCTCGGGCCGGGGAGGGCTCGGGGGAGGGGCGCGGCGGCCCCCGGAGCGCCGGCG |
| | | GCTGTCGAGGCGCGGCGAGCCGCAGCCATTGCCTTTTATGGTAATCGTGCGAGAGGGCGCAGGGACTTCCTTTGT |
| | | CCCAAATCTGTGCGGAGCCGAAATCTGGGAGGCGCCGCCGCACCCCCTCTAGCGGGCGCGGGGCGAAGCGGTGCG |
| | | GCGCCGGCAGGAAGGAAATGGGCGGGGAGGGCCTTCGTGCGTCGCCGCGCCGCCGTCCCCTTCTCCCTCTCCAGC |
| | | CTCGGGGCTGTCCGCGGGGGGACGGCTGCCTTCGGGGGGGACGGGGCAGGGCGGGGTTCGGCTTCTGGCGTGTGA |
| | | CCGGCGGCTCTAGACAATTGTACTAACCTTCTTCTCTTTCCTCTCCTGACAGGGAGTTTAAACAGATAAGTTTGT |
| | | ACAAAAAAGAGAGGTGCCACCATGACAGTGCTGGCTCCTGCTTGGAGCCCTAACAGCTCTCTGCTGCTGCTTCTG |
| | | CTCCTGCTGAGCCCATGTCTGAGAGGCACCCCTGACTGCTACTTCTCTCACAGCCCCATCAGCAGCAACTTCAAA |
| | | GTGAAGTTCCGCGAGCTGACCGACCATCTGCTGAAGGACTACCCTGTGACCGTGGCCGTGAACCTGCAGGATGAG |
| | | AAGCACTGCAAGGCCCTGTGGTCCCTGTTCCTGGCTCAGAGATGGATCGAGCAGCTGAAAACAGTGGCCGGCAGC |
| | | AAGATGCAGACCCTGCTGGAAGATGTGAACACCGAGATCCACTTCGTGACCAGCTGCACCTTCCAGCCTCTGCCT |
| | | GAGTGCCTGAGATTCGTGCAGACCAACATCAGCCACCTTCTCAAGGACACATGCACCCAGCTGCTGGCCCTGAAG |
| | | CCTTGTATCGGCAAGGCCTGCCAGAACTTCTCCAGATGCCTGGAAGTGCAGTGCCAGCCTGACAGCTCTACACTG |
| | | CTGCCTCCAAGAAGCCCTATCGCTCTGGAAGCCACAGAGCTGCCTGAGCCTAGACCTAGACAGTGAGCTTCCACT |
| | | GGATTGTACAATTACcaacAATAAAATATCTTTATTTTCATTACATCTGTGTGTTGGTTTTTTGTGTGCGCTact |
| | | TTCGgtatcacaggagaatttcagGGAGACATTGATTATTGACTAGTTATTAATAGTAATCAATTACGGGGTCAT |
| | | TAGTTCATAGCCCATATATGGAGTTCCGCGTTACATAACTTACGGTAAATGGCCCGCCTGGCTGACCGCCCAACG |
| | | ACCCCCGCCCATTGACGTCAATAATGACGTATGTTCCCATAGTAACGCCAATAGGGACTTTCCATTGACGTCAAT |
| | | GGGTGGAGTATTTACGGTAAACTGCCCACTTGGCAGTACATCAAGTGTATCATATGCCAAGTACGCCCCCTATTG |
| | | ACGTCAATGACGGTAAATGGCCCGCCTGGCATTATGCCCAGTACATGACCTTATGGGACTTTCCTACTTGGCAGT |
| | | ACATCTACGTATTAGTCATCGCTATTACCATGTCGAGGTGAGCCCCACGTTCTGCTTCACTCTCCCCATCTCCCC |
| | | CCCCTCCCCACCCCCAATTTTGTATTTATTTATTTTTTAATTATTTTGTGCAGCGATGGGGGCGGGGGGGGGGGG |
| | | GGGGCGCGCGCCAGGCGGGGCGGGGCGGGGCGAGGGGCGGGGCGGGGCGAGGCGGAGAGGTGCGGCGGCAGCCAA |
| | | TCAGAGCGGCGCGCTCCGAAAGTTTCCTTTTATGGCGAGGCGGCGGCGGCGGCGGCCCTATAAAAAGCGAAGCGC |
| | | GCGGCGGGCGGGAGTCGCTGCGACGCTGCCTTCGCCCCGTGCCCCGCTCCGCCGCCGCCTCGCGCCGCCCGCCCC |
| | | GGCTCTGACTGACCGCGTTACTCCCACAGGTGAGCGGGCGGGACGGCCCTTCTCCTCCGGGCTGTAATTAGCGCT |
| | | TGGTTTAATGACGGCTTGTTTCTTTTCTGTGGCTGCGTGAAAGCCTTGAGGGGCTCCGGGAGGGCCCTTTGTGCG |
| | | GGGGGAGCGGCTCGGGGGGTGCGTGCGTGTGTGTGTGCGTGGGGAGCGCCGCGTGCGGCTCCGCGCTGCCCGGCG |
| | | GCTGTGAGCGCTGCGGGCGCGGCGCGGGGCTTTGTGCGCTCCGCAGTGTGCGCGAGGGGAGCGCGGCCGGGGGCG |
| | | GTGCCCCGCGGTGCGGGGGGGGCTGCGAGGGGAACAAAGGCTGCGTGCGGGGTGTGTGCGTGGGGGGGTGAGCAG |
| | | GGGGTGTGGGCGCGTCGGTCGGGCTGCAACCCCCCCTGCACCCCCCTCCCCGAGTTGCTGAGCACGGCCCGGCTT |
| | | CGGGTGCGGGGCTCCGTACGGGGCGTGGCGCGGGGCTCGCCGTGCCGGGCGGGGGGTGGCGGCAGGTGGGGGTGC |
| | | CGGGCGGGGCGGGGCCGCCTCGGGCCGGGGAGGGCTCGGGGGAGGGGCGCGGCGGCCCCCGGAGCGCCGGCGGCT |
| | | GTCGAGGCGCGGCGAGCCGCAGCCATTGCCTTTTATGGTAATCGTGCGAGAGGGCGCAGGGACTTCCTTTGTCCC |
| | | AAATCTGTGCGGAGCCGAAATCTGGGAGGCGCCGCCGCACCCCCTCTAGCGGGCGCGGGGCGAAGCGGTGCGGCG |
| | | CCGGCAGGAAGGAAATGGGCGGGGAGGGCCTTCGTGCGTCGCCGCGCCGCCGTCCCCTTCTCCCTCTCCAGCCTC |
| | | GGGGCTGTCCGCGGGGGGACGGCTGCCTTCGGGGGGGACGGGGCAGGGCGGGGTTCGGCTTCTGGCGTGTGACCG |
| | | GCGGCTCTAGACAATTGTACTAACCTTCTTCTCTTTCCTCTCCTGACAGGGAGTTTAAACAGATAAGTTTGTACA |
| | | AAAAAGAGAGGTGCCACCATGTGCCCTCAGAAGCTGACCATCAGTTGGTTCGCCATCGTGCTGCTGGTGTCCCCA |
| | | CTGATGGCTATGTGGGAACTCGAGAAGGACGTGTACGTGGTGGAAGTGGACTGGACCCCTGATGCTCCTGGCGAG |
| | | ACAGTGAACCTGACCTGCGACACACCTGAAGAGGACGACATCACCTGGACCAGCGATCAGAGACACGGCGTGATC |
| | | GGCTCTGGCAAGACCCTGACAATTACCGTGAAAGAGTTCCTGGACGCCGGCCAGTACACCTGTCACAAAGGCGGA |
| | | GAGACACTGAGCCACTCTCATCTGCTGCTGCACAAGAAAGAGAACGGCATCTGGTCCACCGAGATCCTGAAGAAC |
| | | TTCAAGAACAAGACCTTCCTGAAGTGCGAGGCCCCTAACTACAGCGGCAGATTCACCTGTAGCTGGCTGGTGCAG |
| | | AGAAACATGGACCTGAAGTTCAACATCAAGTCCTCCAGCAGCAGCCCCGACAGCAGAGCTGTGACATGTGGCATG |
| | | GCTAGCCTGAGCGCCGAGAAAGTGACACTGGACCAGAGAGACTACGAGAAGTACAGCGTGTCCTGCCAAGAGGAC |
| | | GTGACCTGTCCTACCGCCGAGGAAACACTGCCTATCGAGCTGGCCCTGGAAGCCAGACAGCAGAACAAATACGAG |
| | | AACTACTCTACCAGCTTCTTCATCCGGGACATCATCAAGCCCGATCCTCCAAAGAACCTGCAGATGAAGCCTCTG |
| | | AAGAACAGCCAGGTCGAGGTGTCCTGGGAGTACCCTGACAGCTGGTCTACCCCTCACAGCTACTTCAGCCTGAAA |
| | | TTCTTCGTGCGGATCCAGCGCAAGAAAGAAAAGATGAAGGAAACCGAGGAAGGCTGCAACCAGAAAGGCGCTTTC |
| | | CTGGTGGAAAAGACCAGCACCGAGGTGCAGTGCAAAGGCGGCAATGTCTGTGTGCAGGCCCAGGACCGGTACTAC |
| | | AACAGCAGCTGTAGCAAGTGGGCCTGCGTGCCATGCAGAGTCAGATCTGGTGGCGGAGGATCTGGCGGAGGTGGA |
| | | AGCGGCGGAGGCGGATCTAGAGTGATTCCTGTGTCTGGCCCTGCCAGATGCCTGAGCCAGTCTAGAAACCTGCTG |
| | | AAAACCACCGACGACATGGTCAAGACCGCCAGAGAGAAGCTGAAGCACTACTCCTGCACAGCCGAGGACATCGAC |
| | | CACGAGGATATCACCAGGGACCAGACAAGCACCCTGAAAACCTGCCTGCCTCTGGAACTGCATAAGAACGAGAGC |
| | | TGCCTGGCCACCAGAGAAACCAGCTCTACCACAAGAGGCAGCTGTCTGCCTCCTCAGAAAACCAGCCTGATGATG |
| | | ACCCTGTGCCTGGGCAGCATCTACGAGGATCTGAAGATGTACCAGACCGAGTTCCAGGCCATCAACGCCGCTCTG |
| | | CAGAACCACAACCACCAGCAGATCATCCTGGACAAGGGCATGCTGGTGGCTATCGACGAGCTGATGCAGAGCCTG |
| | | AACCATAACGGCGAGACACTGCGGCAGAAGCCTCCAGTTGGAGAGGCCGATCCTTACAGAGTGAAGATGAAGCTG |
| | | TGCATCCTGCTGCACGCCTTCAGCACCAGAGTGGTCACCATCAACAGAGTGATGGGCTACCTGAGCAGCGCCTGA |
| | | GCTTCCACTGGATTGTACAATTACcaacAATAAAATATCTTTATTTTCATTACATCTGTGTGTTGGTTTTTTGTG |
| | | TGCGCTataTTCGgtatcacaggagaatttcagGGAGACATTGATTATTGACTAGTTATTAATAGTAATCAATTA |
| | | CGGGGTCATTAGTTCATAGCCCATATATGGAGTTCCGCGTTACATAACTTACGGTAAATGGCCCGCCTGGCTGAC |
| | | CGCCCAACGACCCCCGCCCATTGACGTCAATAATGACGTATGTTCCCATAGTAACGCCAATAGGGACTTTCCATT |
| | | GACGTCAATGGGTGGAGTATTTACGGTAAACTGCCCACTTGGCAGTACATCAAGTGTATCATATGCCAAGTACGC |
| | | CCCCTATTGACGTCAATGACGGTAAATGGCCCGCCTGGCATTATGCCCAGTACATGACCTTATGGGACTTTCCTA |
| | | CTTGGCAGTACATCTACGTATTAGTCATCGCTATTACCATGTCGAGGTGAGCCCCACGTTCTGCTTCACTCTCCC |
| | | CATCTCCCCCCCCTCCCCACCCCCAATTTTGTATTTATTTATTTTTTAATTATTTTGTGCAGCGATGGGGGCGGG |
| | | GGGGGGGGGGGGGCGCGCGCCAGGCGGGGCGGGGCGGGGCGAGGGGCGGGGCGGGGCGAGGCGGAGAGGTGCGGC |
| | | GGCAGCCAATCAGAGCGGCGCGCTCCGAAAGTTTCCTTTTATGGCGAGGCGGCGGCGGCGGCGGCCCTATAAAAA |
| | | GCGAAGCGCGCGGCGGGCGGGAGTCGCTGCGACGCTGCCTTCGCCCCGTGCCCCGCTCCGCCGCCGCCTCGCGCC |
| | | GCCCGCCCCGGCTCTGACTGACCGCGTTACTCCCACAGGTGAGCGGGCGGGACGGCCCTTCTCCTCCGGGCTGTA |
| | | ATTAGCGCTTGGTTTAATGACGGCTTGTTTCTTTTCTGTGGCTGCGTGAAAGCCTTGAGGGGCTCCGGGAGGGCC |
| | | CTTTGTGCGGGGGGAGCGGCTCGGGGGGTGCGTGCGTGTGTGTGTGCGTGGGGAGCGCCGCGTGCGGCTCCGCGC |
| | | TGCCCGGCGGCTGTGAGCGCTGCGGGCGCGGCGCGGGGCTTTGTGCGCTCCGCAGTGTGCGCGAGGGGAGCGCGG |
| | | CCGGGGGCGGTGCCCCGCGGTGCGGGGGGGGCTGCGAGGGGAACAAAGGCTGCGTGCGGGGTGTGTGCGTGGGGG |
| | | GGTGAGCAGGGGGTGTGGGCGCGTCGGTCGGGCTGCAACCCCCCCTGCACCCCCCTCCCCGAGTTGCTGAGCACG |
| | | GCCCGGCTTCGGGTGCGGGGCTCCGTACGGGGCGTGGCGCGGGGCTCGCCGTGCCGGGCGGGGGGTGGCGGCAGG |
| | | TGGGGGTGCCGGGCGGGGCGGGGCCGCCTCGGGCCGGGGAGGGCTCGGGGGAGGGGCGCGGCGGCCCCCGGAGCG |
| | | CCGGCGGCTGTCGAGGCGCGGCGAGCCGCAGCCATTGCCTTTTATGGTAATCGTGCGAGAGGGCGCAGGGACTTC |
| | | CTTTGTCCCAAATCTGTGCGGAGCCGAAATCTGGGAGGCGCCGCCGCACCCCCTCTAGCGGGCGCGGGGCGAAGC |
| | | GGTGCGGCGCCGGCAGGAAGGAAATGGGCGGGGAGGGCCTTCGTGCGTCGCCGCGCCGCCGTCCCCTTCTCCCTC |
| | | TCCAGCCTCGGGGCTGTCCGCGGGGGGACGGCTGCCTTCGGGGGGGACGGGGCAGGGCGGGGTTCGGCTTCTGGC |
| | | GTGTGACCGGCGGCTCTAGACAATTGTACTAACCTTCTTCTCTTTCCTCTCCTGACAGGGAGTTTAAACAGATAA |
| | | GTTTGTACAAAAAAGAGAGGTGCCACCATGAGACTGCTGCTGCTGACATTCCTGGGCGTGTGCTGTCTGACACCC |
| | | TGGGTTGTCGAAGGCGTGGGAACAGAGGTGCTGGAAGAGTCCAGCTGCGTGAACCTGCAGACCCAGAGACTGCCC |
| | | GTGCAGAAGATCAAGACCTACATCATCTGGGAGGGCGCCATGAGAGCCGTGATCTTCGTGACAAAGAGAGGCCTG |
| | | AAGATCTGCGCCGATCCTGAGGCCAAATGGGTCAAAGCCGCCATCAAGACCGTGGACGGCAGAGCCAGCACCAGA |
| | | AAGAACATGGCCGAGACAGTGCCTACAGGCGCCCAGAGATCTACCAGCACAGCCATCACACTGACCGGCTGAGCT |
| | | TCCACTGGATTGTACAATTACcaacAATAAAATATCTTTATTTTCATTACATCTGTGTGTTGGTTTTTTGTGTGC |
| | | GCTcaaaaaa |

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

1. A method of delivering a nonviral nucleic acid vector into a target retinal cell of an individual, the method comprising:
(a) contacting an electrode with an interior region of an eye of the individual, wherein an extracellular space in the retina of the eye comprises the nonviral nucleic acid vector, wherein the interior region is a subretinal space and/or vitreous humor in the eye; and
(b) while the electrode is contacting the interior region, transmitting pulses of electrical energy through the electrode at conditions suitable for electrotransfer of the nonviral nucleic acid vector into the target retinal cell.

2. The method of claim 1, wherein the nonviral nucleic acid vector was administered by subretinal injection.

3. The method of claim 1, wherein the interior region contacted with the electrode is the subretinal space, and the electrode is a negative electrode.

4. The method of claim 1, wherein the interior region contacted with the electrode is the vitreous humor, and the electrode is a positive electrode.

5. The method of claim 1, wherein the electrode is bipolar and comprises a positive electrode and a negative electrode, the interior region contacted with the positive electrode is the vitreous humor, and the interior region contacted with the negative electrode is the subretinal space.

6. The method of claim 1, wherein the pulses of electrical energy produce a current from 1 mA to 100 mA.

7. The method of claim 1, wherein the pulses of electrical energy are each 1 ms to 200 ms in duration.

8. The method of claim 1, wherein the pulses of electrical energy are transmitted within 1-20 seconds.

9. The method of claim 1, wherein the nonviral nucleic acid vector is a naked nucleic acid vector.

10. The method of claim 1, wherein the nonviral nucleic acid vector is a DNA vector.

11. The method of claim 10, wherein the DNA vector is a naked circular DNA vector.

12. The method of claim 9, wherein the naked nucleic acid vector is a synthetic circular DNA vector, a plasmid DNA vector, a minicircle DNA vector, or a linear closed-ended DNA vector.

13. The method of claim 12, wherein the naked nucleic acid vector is a synthetic circular DNA vector.

14. The method of claim 13, wherein the synthetic circular DNA vector is supercoiled.

15. The method of claim 1, wherein the nonviral nucleic acid vector was administered by suprachoroidal injection.

16. The method of claim 1, wherein the nonviral nucleic acid vector is encapsulated in a nanoparticle or a microparticle.

17. The method of claim 1, wherein the nonviral nucleic acid vector is expressed in the target retinal cell.

18. The method of claim 1, wherein the nonviral nucleic acid vector is greater than 8 kb.

19. The method of claim 1, wherein the target retinal cell is a photoreceptor cell or an RPE cell.

20. The method of claim 1, wherein the nonviral nucleic acid vector encodes a therapeutic protein, and wherein the therapeutic protein is expressed in the target retinal cell.

21. The method of claim 20, wherein expression of the therapeutic protein persists for at least one month.

22. The method of claim 20, wherein the therapeutic protein is encoded by a sequence that is greater than 8 kb.

23. The method of claim 20, wherein the therapeutic protein is ABCA4, MYO7A, BEST1, CFH, CEP290, USH2A, ADGRV1, CDH23, CRB1, PCDH15, RPGR, ABCC6, RIMS1, LRPS, CC2D2A, TRPM1, C3, IFT172, COL11A1, TUBGCP6, KIAA1549, CACNA1F, PRPF8, VCAN, USH2A, HMCN1, RPE65, NR2E3, NRL, RHO, RP1, RP2, or OFD1.

24. The method of claim 20, wherein the target retinal cell is a photoreceptor cell or an RPE cell.

25. The method of claim 20, wherein the method is for treating or preventing an ocular disease or disorder in the individual.

26. The method of claim 20, wherein the therapeutic protein is ABCA4, and the method is for treating an ABCA4-associated retinal dystrophy.

27. The method of claim 26, wherein the ABCA4-associated retinal dystrophy is Stargardt's disease or rod-cone dystrophy.

28. The method of claim 27, wherein the nonviral nucleic acid vector comprises a nucleic acid sequence with at least 95% sequence identity to SEQ ID NO: 19.

29. The method of claim 28, wherein the nonviral nucleic acid vector comprises the nucleic acid sequence of SEQ ID NO: 19.

30. The method of claim 20, wherein the individual is a human.
